(19) United States
(12) Patent Application Publication
(10) Pub. No.: US 2026/0283554 A1
(43) Pub. Date: Sep. 24, 2026

(54) ANALYTE MONITORING SYSTEMS WITH ON-BODY DEVICES HAVING IMPROVED WEAR DURATION

(71) Applicant: ABBOTT DIABETES CARE INC., Alameda, CA (US)

(72) Inventors: Sachin M. Shenoi, Cupertino, CA (US); Vivek S. Rao, Alameda, CA (US); Stephen T. Pudjijanto, San Ramon, CA (US); Udo Hoss, San Ramon, CA (US); Clive Matthew Bates, San Ramon, CA (US); Jean-Pierre J. Cole, Tracy, CA (US); Matthew Simmons, Pleasanton, CA (US); Johnny Chang, San Bruno, CA (US); Peter M. Voit, Dublin, CA (US)

(21) Appl. No.: 19/571,744

(22) Filed: Mar. 19, 2026

Related U.S. Application Data
(60) Provisional application No. 63/774,585, filed on Mar. 19, 2025.
(60) Provisional application No. 63/831,052, filed on Jun. 26, 2025.
(60) Provisional application No. 63/840,507, filed on Jul. 8, 2025.
(60) Provisional application No. 63/912,265, filed on Nov. 5, 2025.

(51) Int. Cl. A61B 5/00; A61B 5/145; A61B 5/1486

(52) U.S. Cl. CPC A61B 5/6833; A61B 5/14517; A61B 5/14532; A61B 5/14865

(57) ABSTRACT

An on-body device for monitoring an analyte level of a user has a housing. The housing has a vent inlet and a vent outlet that allow passage of water vapor therethrough. Examples of moisture permeable seals, solid foam seals, and fibrous seals are provided. The housing has a base adapted to permit egress of moisture from regions adjacent the housing. The base may include surface channels to facilitate moisture transport. Directional and non-directional surface textures are provided. An adhesive patch for securing the on-body device and having a plurality of patch portions is disclosed as is a reinforcement device for reinforcing attachment. The reinforcement device can include a reinforcement leg or a reinforcement band. The adhesive patches can include a non-isotropic layer with an opening and/or a polymeric layer. Adhesive patches with support structures, and variations of support structures, are described. Adhesive patches with various liner configurations are described.

### CROSS-REFERENCE TO RELATED APPLICATIONS

This application claims the benefit of and priority to U.S. Provisional Application No. 63/774,585, filed Mar. 19, 2025, U.S. Provisional Application No. 63/831,052, filed Jun. 26, 2025, U.S. Provisional Application No. 63/840,507, filed Jul. 8, 2025, and U.S. Provisional Application No. 63/912,265, filed Nov. 5, 2025, all of which are incorporated by reference herein in their entireties and for all purposes.

### FIELD

The present disclosure relates generally to analyte monitoring systems. In particular, the present disclosure relates to analyte sensors for analyte monitoring, on-body devices for use with such analyte sensors, analyte monitoring systems comprising such on-body devices, devices for inserting such analyte sensors and applying such on-body devices, and related methods of manufacturing and using the same.

### BACKGROUND

The human body is a complex biological system in which many chemical substances are involved in a myriad of biological processes. An understanding of the concentration level of these substances and how they vary can provide beneficial information about a person's body. Systems have been developed to conveniently monitor concentration levels of target substances, or analytes, and provide these levels over time for consideration, analysis, and/or further action. These systems typically include an electronic on-body device that is placed on the person's body and is operable in conjunction with a sensor for measuring the concentration levels of the analyte while that analyte is within the body. These systems often operate with a receiver unit that can receive analyte concentration levels from the on-body device and display them to the user in a convenient, aesthetic, and informative manner. The sensor component of these analyte monitoring systems can have various configurations that, while operating in conjunction with the on-body device, can be positioned wholly in vivo (within the human body), partially in vivo, or wholly ex vivo to measure the analyte while within the body. These on-body analyte monitoring systems are advantageous over devices used for discrete testing with in vitro techniques such as those that rely on removing a fluid sample from the body (e.g., by a fingerstick) and testing for analyte concentration outside of the body, such as with a test strip and meter. In vitro systems can be inconvenient and painful, and only provide one measurement when the test is performed which leaves large gaps in time for which no measurement readings are available.

A single on-body analyte monitoring system can measure concentration levels for one, two, three, or more different analytes. These analytes can be selected from a range of numerous different analyte types having beneficial relevance for a variety of states pertaining to the human body. These states include medical conditions impacting physical and/or mental health, states of well-being, response to physical exertion, states of fitness, a state of impairment, response to diet, response to physical or mental stress, and/or response to environment generally, to name a few.

On-body analyte monitoring systems are often used by individuals that have or may be susceptible to the medical condition diabetes mellitus (often referred to as diabetes), which is a serious disease where the body does not produce or properly utilize insulin. Insulin is a hormone produced by the pancreas that regulates the substance blood glucose (also referred to as blood sugar). In particular, when blood glucose levels rise (e.g., after a meal), insulin lowers the blood glucose levels by facilitating glucose to move from the blood into the body cells. When the pancreas does not produce sufficient insulin (a condition known as Type I diabetes) or body cells do not properly utilize insulin (a condition known as Type II diabetes), the glucose remains in the blood, resulting in hyperglycemia (high glucose). The inability to control glucose levels can also lead to hypoglycemia (low glucose). These are urgent problems and can even be life-threatening.

The vast and uncontrolled fluctuations in glucose levels in people suffering from diabetes can cause long-term serious health complications. For example, complications may include blindness, kidney failure, and nerve damage. Additionally, it is known that diabetes is a factor in accelerating cardiovascular diseases such as atherosclerosis (hardening of the arteries), leading to stroke, coronary heart disease, and other diseases. It is therefore important to monitor and control glucose levels to manage diabetes.

The body's ketone levels may be dysregulated in people suffering from diabetes. Diabetic ketoacidosis (DKA) is a condition where lack of insulin can lead to build up of harmful ketones in the blood. The monitoring of a ketone as an analyte can thus help manage diabetes; however, ketone monitoring is beneficial for other reasons unrelated to diabetes. For example, individuals with non-diabetic medical conditions that can lead to ketoacidosis, such as eating disorders, gastrointestinal and digestive disorders such as those involving vomiting and/or diarrhea for an extended period, or alcohol use disorders, can benefit from ketone monitoring. Ketone monitoring is also useful for individuals on ketogenic diets, carb-cycling diets, and other diets low in carbohydrates, as well as individuals that are pregnant or performing rigorous exercise routines.

Another analyte that is beneficial to monitor is lactate (or alternatively lactic acid). Lactate monitoring can provide insight for individuals that desire to analyze exercise performance by examining lactate thresholds, peak tolerance, and fatigue. Lactate monitoring can also be used to assess medical conditions such as heart failure, liver disease, and others.

Ethanol (ethyl alcohol, commonly referred to as alcohol) is yet another analyte that is beneficial to monitor. Ethanol is the primary alcohol found in adult alcoholic beverages. On-body analyte monitoring systems capable of measuring in vivo ethanol and/or its metabolite levels can be useful in determining cognitive impairment, which in turn can assist in avoiding operation of motor vehicles or other machinery while impaired. Such systems can also be used in the context of assisting individuals to achieve sobriety as well as in a legal enforcement context.

The on-body device of the system is typically attached to the subject's skin, often in a location on the arm, abdomen, or upper buttock. The amount of time the on-body device has been or will be active on the body is referred to as the wear duration. The full completed extent of time the on-body device was active on the body, from assumption of an activated state on the body to termination of the on-body device's functional measurement capability on the body (e.g., such as by expiration, fault, or removal) is referred to as the completed wear duration. In an effort to ensure reliable measurements, some systems with partially implanted sensors may have a maximum duration the on-body device and sensor are permitted to operate together while on the subject's body, and this is referred to as the maximum wear duration. The maximum wear duration can be enforced by the software of a system component, such as the on-body device, the sensor itself in some cases, and/or a receiver unit, so that new analyte level measurements can no longer be taken and/or displayed after the maximum wear duration has been reached. The maximum wear duration varies based on the implementation of the sensor and on-body device but is in the range of 7 to 15 days for conventional partially in vivo systems, and longer for wholly in vivo systems. The maximum wear duration may include an initial warm-up period, usually in the range of one hour to one day, during which the on-body device is active on the body but the system is not permitted to output new analyte measurements to the subject. Some but not all systems require a warm-up period, for example, to allow the body's biological response to the sensor's presence to subside or to allow the inputting of in vitro measurements to calibrate the sensor.

System developers design the on-body device and sensor to remain on the body while generating reliably accurate analyte measurements for as long of a wear duration as-is practical and commercially viable. Conditions that the on-body device and sensor are subjected to will vary from one subject to the next due to biological conditions in and on the subject's body and environmental conditions the system is exposed to. This variance leads to unpredictability in determining the likely completed wear duration the on-body device and sensor will achieve before requiring replacement. Nevertheless, system developers can estimate, with a high degree of confidence derived from analysis and testing, a wear duration during which the on-body device and sensor will remain operable if used within a range of operating conditions specified by the developer. This estimate is referred to herein as the designed wear duration.

The designed wear duration of the on-body device can be limited by the attachment life, which is the amount of time the attachment structure holds the on-body device to the body. This attachment structure can be an adhesive patch, a non-adhesive wrap or bandage, a combination of both or otherwise. In the case of on-body devices (“OBD”) adhesively attached to the skin, a significant factor in determining the attachment life is the amount of time sufficient adhesive coupling with the skin can be maintained. The designed wear duration of the on-body device can also be limited by the power supply life, which is the amount of time the power supply can provide the requisite power for operation of the on-body device (and also the sensor if the on-body device is responsible for powering the sensor). The power supply life is dependent on the power capacity of the power supply and the power consumption by the on-body device electronics, as well as the power consumption of the sensor in cases where the sensor does not have its own power supply. For on-body devices having a partially implanted sensor or wholly ex vivo sensor, the designed wear duration can also be limited by the sensor life, which is the length of time the sensor can produce a reliably accurate measurement signal, which in turn is often limited by either or both of the performance of the electrochemical or other primary technique by which the sensor generates the measurement signal and, for partially and wholly implanted sensors, the body's biological response to the presence of the sensor which can degrade the measurement signal or lead to irritation and/or infection.

Moisture accumulation between the device housing and the skin, or within the adhesive patch itself (e.g., from perspiration, showering, swimming, or other environmental exposure), can adversely affect the wear duration. In particular, the moisture can weaken adhesive properties, potentially leading to loosening or complete detachment of the device. Moisture accumulation can also lead to an increase in the shedding of dead skin cells. As a result, the adhesive becomes coupled to dead skin cells, rather than with intact portions of skin, leading to peeling away of the patch.

Induration of the skin, wherein the skin becomes thicker and harder due to an inflammatory process, can also limit wear duration. In particular, by virtue of thickening of the skin, induration can lead to a lifting of the on-body device, which weakens the adhesive bond. Induration can be caused by a number of factors, which can include moisture accumulation and an uneven base surface of the on-body device (such as in the region of the sensor). Induration is otherwise undesirable for the user, due to discomfort and appearance.

Another limiting factor on wear duration can be the strength of the bond between the adhesive patch and the on-body device housing. The underside of the on-body device housing includes features to accommodate and introduce the sensor, for example, which reduce the surface area available for adhesion. A peeling of the adhesive patch from a peripheral edge of the housing can quickly propagate as exposed adhesive coated surfaces may catch on clothing or other material. Such catches or snags can lead to further stresses on the adhesive patch and/or on the on-body device, such that the peeling action propagates, and the on-body device detaches.

It is desirable to improve the designed wear duration of on-body devices by making the designed wear duration more robust and reliable, as well as by extending the designed wear duration. This heightens convenience for the user by lessening the time between replacements. Replacing an on-body device can be inconvenient since it requires the user to be available and have a replacement on-body device on hand at the required time. It is beneficial to reduce the frequency at which this is necessary. Providing a more robust and/or longer designed wear duration may also reduce costs for the user since fewer on-body devices would be required over a given period of time. This can also reduce waste as well as energy and resource consumption involved in production for a given user. On-body devices with improved designed wear durations can also exemplify higher quality and/or sophistication in the marketplace, which can lead to greater revenue for the system developer.

Thus, a need exists for on-body devices for analyte monitoring having improved designed wear durations.

### SUMMARY

The scope of the present disclosure is defined by the appended claims.

The inventors have surprisingly figured out that the wear time of an on-body device can be significantly extended through a number of systems, devices, methods, and aspects outlined herein.

For example, during testing, the inventors figured out that if an on-body device is positioned on the wearer's body while exposed to persistent moisture then the on-body device is likely to detach from the wearer's body prematurely as discussed. As will be described in more detail below, the inventors have discovered that several approaches are effective at minimizing the accumulation of moisture, the duration of time the on-body device is exposed to a moisture-bearing environment, or both, by facilitating moisture egress.

According to various aspects of the present disclosure, there is generally provided an on-body device having a vent inlet and a vent outlet to allow passage of water vapor, an on-body device with a vent passageway, an on-body device with a moisture permeable seal, an on-body device with a solid foam seal, an on-body device with a fibrous seal, methods of use of such on-body devices, systems having such on-body devices, and methods of manufacturing the same.

According to an aspect of the present disclosure, there is provided: an on-body device for monitoring an analyte level of a user, comprising: a housing, comprising: a first surface comprising an aperture and a vent inlet; and a second surface comprising a vent outlet; wherein the vent inlet is fluidly connected to the vent outlet through the housing to allow passage of water vapor from the first surface to the second surface; an adhesive patch attached to the housing and configured to attach the housing to skin of the user; sensor electronics, wherein the housing is configured to retain the sensor electronics; and an analyte sensor, comprising: a first portion configured to be in electrical contact with the sensor electronics; and a second portion configured to be positioned in the body of the user for monitoring an analyte level of the user; wherein the analyte sensor is configured to extend through the aperture for positioning the second portion of the analyte sensor in the body of the user.

The on-body device may alternatively be referred to as an on-body unit, a sensor electronics device, a sensor control device, or an analyte monitoring device. The on-body device is generally provided for monitoring an analyte level of the user. For example, the on-body device may be configured to monitor an analyte level of the user. In particular, the analyte level of the user may be determined by the on-body device (e.g., by using the analyte sensor to obtain a signal corresponding to the analyte level). The analyte level may be monitored over time, preferably on a continuous basis. The analyte level may be referred to as the concentration of the analyte (e.g., in the user's blood). The user is preferably the wearer of the on-body device (the wearer is also referred to herein as the subject).

The on-body device comprises a housing. The housing may be referred to as a structure or a body. In some examples, the housing may be referred to as an enclosure. The housing comprises a first surface. The first surface may be an external surface of the housing. As explained below, in some examples, the first surface may be a base surface of the housing. The first surface comprises an aperture. The aperture may be referred to as a first aperture or a sensor aperture. The aperture may be a hole through the first surface. The analyte sensor is configured to extend through the aperture for positioning in the body. Thus, the first surface may be configured to face the skin. The sensor aperture may be any shape (e.g., circular, oval, polygonal, etc.) and located at any location of the first surface.

The first surface also comprises a vent inlet. The vent inlet may be referred to as an aperture (e.g., a second aperture), an opening, or a hole. The vent inlet may be a hole through the first surface, in addition to the aperture. Thus, the vent inlet is arranged in the same surface (the first surface) that the analyte sensor extends from. The first surface may comprise at least one vent inlet.

The housing also comprises a second surface. The second surface may be an external surface of the housing. As explained below, in some examples, the second surface may be a top surface, a side surface, or an intermediate surface of the housing. The second surface is a different surface to the first surface. The second surface comprises a vent outlet. The vent outlet may be referred to as an aperture (e.g., a third aperture), an opening, or a hole. The vent outlet may be a hole through the second surface. The vent inlet is fluidly connected to the vent outlet. Thus, fluid is capable of flowing from the vent inlet to the vent outlet. In particular, the housing is configured to allow water vapor to pass (e.g., flow) from the vent inlet to the vent outlet. The second surface may comprise at least one vent outlet.

By providing a vent inlet in the first surface of the housing which is fluidly connected to a vent outlet in the second surface, water vapor can be released from the first surface to the second surface. This may allow water vapor to be released from the housing. In particular, water collecting on the first surface or in the adhesive patch (which may be adjacent and optionally attached to the first surface) can be removed, for example by evaporation and passing the resulting water vapor through the vent inlet and out of the vent outlet for release from the housing and into the atmosphere. In this manner, the housing can be vented or ventilated. This reduces build-up of water on the first surface.

An adhesive patch is attached to the housing. Preferably, the adhesive patch is attached to the first surface. For example, the adhesive patch may be attached to the base surface. The adhesive patch can therefore be configured to attach the first surface of the housing to the skin. For example, one side of the adhesive patch may be attached to the housing (e.g., to the first surface) and the opposite side may be configured to be attached to the skin. If the adhesive fails to retain the on-body device on the skin, then the on-body device may need to be replaced. The adhesive can be affected over time, for example due to moisture (e.g., water). Moisture can collect between the adhesive patch and the housing of the on-body device or between the adhesive patch and the skin, for example due to sweating or from water ingress (e.g., from showering, swimming, or rain). In some examples, moisture can become trapped due to the lack of air flow between the housing and the skin. In particular, moisture can be trapped between the first surface and the skin (e.g., between the first surface and the adhesive patch, or between the adhesive patch and the skin). The trapped moisture can then affect the adhesive properties of the adhesive patch and/or the health of the skin through a phenomenon known as maceration. This may lead to degradation of the adhesive, degradation of the skin, loosening of the adhesive patch, and ultimately may lead to complete detachment of the housing from the skin. The adhesive force between the adhesive patch and the skin is often less than between the housing and the adhesive patch, since the adhesive patch is configured to be removed from the skin after use. Accordingly, it is typically this adhesive that is prone to failing first, meaning the housing (with the adhesive patch) detaches from the skin.

Providing a vent inlet and a vent outlet to remove water vapor from the housing can prevent degradation of the adhesive patch. This can increase the time that the adhesive remains effective. In turn, this can allow the wear duration of the on-body device to be increased. For example, the wear duration may be 30 days or longer.

Water vapor is the gaseous form of water. In some examples, other fluids may be allowed to pass from the vent inlet to the vent outlet. For example, the on-body device may additionally be configured to allow liquid water to pass (e.g., in through the vent inlet and out through the vent outlet). In some examples, the water vapor may be referred to as moisture.

The housing is configured to retain the sensor electronics. For example, the housing may be configured to hold the sensor electronics. In some examples, the sensor electronics may be arranged within the housing. In particular, the sensor electronics may be enclosed within the housing, for example between the first surface and the second surface. In other examples, the sensor electronics may be attached to the housing, and the sensor electronics may be held in place by the housing. For example, the housing may act as a receptacle for receiving the sensor electronics. In some examples, the on-body device may be configured to receive the sensor electronics, such as after insertion of the analyte sensor.

The sensor electronics preferably refers to electronics associated with operation of the analyte sensor. For example, the sensor electronics may be configured to receive a signal from the analyte sensor corresponding to the analyte level. The sensor electronics may be configured to process the signal (e.g., the sensor electronics may comprise a processor). The sensor electronics may be configured to transmit data corresponding to the signal, for example to a receiver unit (e.g., the sensor electronics may comprise a transmitter). The sensor electronics may comprise a power source, such as a battery, for powering the sensor electronics. The sensor electronics may also comprise additional components and/or perform additional functionality as described herein (e.g., see FIGS. 1R-1 and 1R-2).

The on-body device also comprises an analyte sensor. The first portion of the analyte sensor is configured to be in electrical contact with the sensor electronics. In some examples, the first portion may be in physical contact with the sensor electronics. Preferably, the first portion of the analyte sensor is coupled to the sensor electronics (e.g., during manufacture). In other examples, the first portion of the analyte sensor may be configured to be coupled to the sensor electronics (e.g., by the user, such as before, during, or after insertion of the analyte sensor into the body).

The second portion of the analyte sensor is configured to be positioned in the body of the user. Preferably, (at least part of) the second portion of the analyte sensor is configured to be positioned beneath the skin of the user. Thus, the analyte sensor may be a subcutaneous analyte sensor. In particular, the second portion of the analyte sensor may be configured to be positioned in contact with a bodily fluid of the user. Preferably, the bodily fluid is interstitial fluid. For example, an inserter may be used to insert the analyte sensor into the body. The inserter may also be used to position the housing on the skin. For example, the analyte sensor and the housing may be integrated (e.g., during manufacture, or by the user prior to insertion), and the combined analyte sensor and housing are inserted/applied by the inserter. The first portion of the analyte sensor may remain outside of the body whilst the second portion may be inserted into the body. For example, the first portion of the analyte sensor may be positioned within the housing. Thus, the analyte sensor may be referred to as a partially insertable analyte sensor. The analyte sensor may be referred to as a transcutaneous analyte sensor.

By placing the second portion of the analyte sensor in the body of the user, the analyte level of the user can be monitored. For example, the second portion of the analyte sensor may be configured to sample the analyte level of the user. The analyte sensor may provide a signal to the sensor electronics corresponding to the sampled analyte level of the user. This signal may then be used to monitor the analyte level. In some examples, the analyte sensor is an electrochemical sensor. For example, electrodes may be used to generate the signal corresponding to the analyte level. In this manner, the on-body device may be configured to monitor the analyte level of the user.

In some examples, the analyte sensor is a glucose sensor. This allows the glucose level of the user to be monitored. In other examples, other analytes may be monitored, as described herein.

The analyte sensor is configured to extend through the aperture in the first surface. The analyte sensor may protrude from the housing. In particular, part of the analyte sensor may protrude beyond the first surface (e.g., in a proximal direction). This can allow the second portion of the analyte sensor to be positioned in the body of the user. For example, when the on-body device is placed on the skin (for example, the base surface of the housing may be placed on the skin, e.g., attached via an intervening adhesive patch), the analyte sensor may be partially inserted into the body. The analyte sensor may extend through the base surface. For example, the analyte sensor may be configured to extend through the aperture to position the second portion of the analyte sensor in the body.

In some examples, the first surface is a base surface of the housing. In some examples, the first surface may be the base of the housing. In some examples, the base may comprise more than one surface (e.g., a further surface in addition to the first surface). The first surface may be referred to as the bottom surface of the housing (e.g., the surface configured to face the skin). The base surface may be the surface configured to face the skin. For example, the adhesive patch may be attached to the base surface. In some examples, the base surface may be planar.

In some examples, the second surface is a top surface of the housing. Thus, the vent outlet may be in the top surface. In some examples, a side surface may be arranged between the first surface and the second surface (e.g., the top surface may be connected to the side surface and the side surface may be connected to the base surface). In this case, the side surface may be referred to as a third surface. In other examples, the top surface may be connected to the first surface (e.g., the top surface may be connected to the base surface), and the side surface may not be present. Providing the vent outlet in the top surface may encourage airflow into the vent outlet for improved moisture egress. For low-profile shapes of housing, the distance between the vent inlet and the vent outlet may also be small compared to the distance to the side surface (e.g., to the periphery of the base surface). This may also simplify design since straight (e.g., axial) vent passageways (e.g., cylindrical or other shape) may be provided between the vent inlet and the vent outlet through the housing, as explained below.

In some examples, the second surface is a side surface of the housing. Thus, the vent outlet may be in the side surface. In some examples, the side surface may be connected to the first surface (e.g., the side surface may be connected to the base surface). The side surface may then be connected to the top surface. In this case, the top surface may be referred to as a third surface. Providing the vent outlet in the side surface may avoid disrupting sensor electronics. For example, the vent outlet may be positioned proximally of (e.g., below) sensor electronics (e.g., below a PCB). This may mean that water vapor passing between the vent inlet and the vent outlet is not routed through the sensor electronics, which increases the available space for electronics. This can reduce the size of the housing or allow for an increase in the size of the sensor electronics, such as allowing for increasing processing or transmission capabilities or increased battery capacity (which can ultimately increase wear duration).

In some examples, the housing may include an intermediate surface. For example, the intermediate surface may be arranged between the first surface and the side surface. In another example, the intermediate surface may be arranged between the side surface and the top surface. In some examples, the second surface may be the intermediate surface. For example, the vent outlet may be in the intermediate surface. In other examples, the intermediate surface may be a fourth surface. The benefits described in relation to the top surface and/or the side surface may be applicable to the intermediate surface.

In some examples, the vent inlet is spaced away from a periphery of the first surface. Thus, the vent inlet may be spaced away from the periphery of the base surface. A periphery of the first surface may be a periphery of the housing. For example, the vent inlet may not be contiguous with a periphery (e.g., an outer edge) of the first surface. The first surface may extend fully around the periphery of the vent inlet. Thus, the vent inlet may not intersect with the periphery of the first surface. This avoids a reduction in the surface area of the first surface around the periphery of the first surface that can be in contact with the adhesive patch. For example, if the vent inlet intersects the periphery of the first surface, the surface area of the first surface at the periphery may be reduced, and in turn can lead to a reduction in the adhesive effectiveness. Accordingly, providing the vent inlet spaced away from the periphery may increase the effectiveness of the adhesive patch and allow for increased wear duration.

In some examples, the vent outlet is aligned with the vent inlet along an axis perpendicular to the first surface. For example, the vent outlet may be aligned with the vent inlet along an axis perpendicular to the base surface. In particular, the vent outlet may be in the top surface and the vent inlet may be in the base surface. This can reduce the distance between the vent inlet and the vent outlet, increasing the effectiveness of moisture egress and minimizing impact on the space in the housing. This may also simplify design since straight (e.g., axial) vent passageways (e.g., cylindrical or other shape) may be provided. In other examples, the vent outlet may be offset relative to the vent inlet. For example, the vent passageway may be angled or curved.

In some examples, the vent outlet is smaller than the vent inlet. For example, a width of the vent outlet may be less than a width of the vent inlet. The width may refer to a diameter, such as where the cross-sectional area is circular. Providing a relatively smaller vent outlet can reduce the risk of water entering the vent outlet from the environment (e.g., from showering, swimming, or rain), in turn reducing moisture collecting on the first surface, and thus allowing for improved wear duration. Providing a relatively larger vent inlet can also increase the surface area available for collection of moisture (e.g., from the adhesive patch or the skin), thereby increasing the potential for moisture egress.

In other examples, the vent outlet may be the same size as the vent inlet. In other examples, the vent outlet may be larger than the vent inlet. This may improve airflow and encourage evaporation of water on the first surface. Preferably, the vent inlet is larger than the vent outlet.

In some examples, the vent inlet comprises a width of at least 0.1 mm, preferably at least 0.2 mm. As used herein “mm” refers to units of millimeters. For example, the width may be about 0.5 mm. This is beneficial to provide sufficient area to collect moisture.

In some examples, the vent inlet comprises a width of less than 5 mm. This is beneficial to increase the contact area with the adhesive patch and improve the mechanical connection. This can provide improved securing for extended wear. In some examples, the vent inlet comprises a width of less than 2 mm, preferably less than 1 mm. In a preferred example, the vent inlet has a width of about 0.5 mm. For example, the width may be between 0.2 mm and 5 mm, preferably between 0.2 mm and 2 mm, or more preferably between 0.2 mm and 1 mm.

In some examples, the vent outlet comprises a width of at least 0.1 mm, preferably at least 0.2 mm. For example, the width may be about 0.5 mm. This is beneficial to provide sufficient area to release water vapor.

In some examples, the vent outlet comprises a width of less than 1 mm. This can reduce the risk of water entering the vent outlet from the environment, improving the adhesive effectiveness. In a preferred example, the vent outlet has a width of about 0.5 mm. For example, the width may be between 0.2 mm and 5 mm, preferably between 0.2 mm and 2 mm, or more preferably between 0.2 mm and 1 mm.

In some examples, the housing comprises a vent passageway between the vent inlet and the vent outlet. The vent passageway may be referred to as a vent passage, a vent conduit, a vent duct, a vent lumen, or a vent. The vent passageway can extend through the housing. For example, the vent inlet may be connected to the vent outlet via the vent passageway. Water vapor from the first surface can therefore pass along the vent passageway for release from the housing. For example, the vent passageway may extend from the base surface to the second surface (e.g., the top surface, the side surface, or the intermediate surface). Providing a vent passageway may allow the water to be separated from the interior of the housing, such as from the sensor electronics.

In some examples, the sensor electronics are sealed from the vent passageway. Water vapor that passes through the vent passageway can be prevented from interfering with the sensor electronics and causing damage. For example, the vent passageway may comprise a passageway wall (e.g., cylindrical wall or other shape) that extends between the vent inlet and the vent outlet, where the passageway wall defines an interior space through which the water vapor can pass. The passageway wall can then prevent the water vapor from passing into the interior of the housing (e.g., into contact with the sensor electronics).

In some examples, the vent passageway extends through the sensor electronics. For example, the vent passageway may extend through a PCB of the sensor electronics. This can allow the vent passageway to pass through the housing, for example in contrast to routing the vent passageway around the edge of the sensor electronics (e.g., around the edge of a PCB). For example, the sensor electronics (e.g., a PCB) may comprise an aperture for receiving the vent passageway. The aperture may be positioned in the PCB to avoid components, such as a processor, battery, or antenna.

In some examples, the vent passageway has a circular cross section. For example, the vent inlet and/or the vent outlet may be circular. For example, the vent passageway may be cylindrical in shape. In other examples, the cross-sectional shape of the vent passageway and/or the shape of the vent inlet and/or the vent outlet may be different, such as oval or square. In some examples, the vent passageway may extend perpendicularly to the base surface (e.g., the vent passageway may be a right cylinder or other similar shape with different cross section). In other examples, the vent passageway may extend at an angle (e.g., less than) 90° to the base surface (e.g., the vent passageway may be an oblique cylinder or other similar shape with different cross section).

In some examples, the vent passageway has a constant cross section in an axis perpendicular to a length of the vent passageway. In other examples, the cross-sectional size of the vent passageway may change along the length. For example, the vent passageway may taper (e.g., forming a conical shape). In other examples, the vent passageway may have a stepped configuration with multiple sections of different sizes. For example, a portion of the vent passageway with a larger width may be arranged proximally of (e.g., below) the sensor electronics (e.g., between the sensor electronics and the first surface). This may allow the vent inlet to be larger for maximizing collection of moisture.

In some examples, the width of the vent passageway through the sensor electronics may be smaller than a largest width of the vent passageway. This may reduce the impact on the sensor electronics. For example, the changing width of the vent passageway may be achieved by providing a tapered or stepped configuration. For example, the width of the vent passageway through the sensor electronics may be smaller than the width of the vent inlet and/or the vent outlet. The width of the vent passageway through the sensor electronics may be a smallest width of the vent passageway.

In some examples, the aperture in the first surface is connected to the vent passageway to form a sensor passageway through the housing between the aperture and the vent passageway, and wherein the vent outlet is configured to receive a sharp for positioning the second portion of the analyte sensor in the body of the user. For example, the sensor passageway may be connected to the vent passageway. The sensor passageway may be connected to the vent passageway at a point along the vent passageway between the vent inlet and the vent outlet. In this manner, a sharp can be inserted through the vent outlet, along a section of the vent passageway, into the sensor passageway, and through the aperture in the first surface. In some examples, the vent outlet is configured to receive a sharp to position the second portion of the analyte sensor in the body. The vent outlet may be configured to receive a sharp, wherein the sharp is configured to insert the analyte sensor into the body of the user. The sharp may be used to insert the analyte sensor into the body of the user. The sharp can then be retracted from the housing. For example, an inserter for advancing (and optionally retracting the sharp) may be used with the on-body device, as described herein. The aperture in the first surface may be aligned with the vent outlet along an axis perpendicular to the first surface. This allows the sharp to be inserted at an angle perpendicular to the skin.

In some examples, the second surface comprises a second aperture, wherein the aperture in the first surface is connected to the second aperture in the second surface to form a sensor passageway through the housing, and wherein the second aperture is configured to receive a sharp for positioning the second portion of the analyte sensor in the body of the user. The aperture in the first surface may be referred to as a first aperture. A sharp can be inserted through the second aperture, along the sensor passageway, and through the first aperture. The sensor passageway may be separate from the vent passageway, and these may not be connected. In some examples, the second aperture is configured to receive a sharp to position the second portion of the analyte sensor in the body. The second aperture may be configured to receive a sharp, wherein the sharp is configured to insert the analyte sensor into the body of the user. The second aperture may be any shape (e.g., circular, oval, polygonal, etc.) and located at any location of the second surface. The shape and placement of the second aperture is generally unrelated to analyte sensing functionality. The second aperture may be arranged in the top surface. The first aperture may be aligned with the second aperture along an axis perpendicular to the first surface. This allows the sharp to be inserted at an angle perpendicular to the skin. In other examples, the second aperture may be arranged along an axis that is not perpendicular to the first surface. The second aperture may be arranged in the side surface or the intermediate surface. The second aperture may be arranged in a different surface to the second surface. For example, the vent outlet may be arranged in the second surface and the second aperture may be arranged in a third surface. In one example, the vent outlet may be arranged in the side surface and the second aperture may be arranged in the top surface.

In some examples, the housing comprises a vent compartment, wherein the vent inlet is fluidly connected to the vent compartment, and wherein the vent compartment is fluidly connected to the vent outlet. This may provide a larger volume for collection of water vapor for release, whilst minimizing the impact on space in the housing, in particular on the sensor electronics. For example, the vent compartment may be provided between the sensor electronics and the first surface (e.g., the base surface). In this manner, the water vapor may not be passed through the sensor electronics. Instead, the vent compartment may comprise an interior space of the housing. The vent compartment may be sealed from the sensor electronics. The vent compartment may comprise one or more vent inlets in the first surface for allowing water vapor into the vent compartment. The vent compartment may extend to a periphery of the first surface. For example, for a disc shaped housing, the vent compartment may comprise a disc shaped portion at the proximal side of the housing. The vent compartment may comprise one or more vent outlets in the surface defining a periphery (e.g., side) of the vent compartment. For example, (part of) the side surface may define a periphery of the vent compartment. In other examples, (part of) the top surface may define the periphery of the vent compartment.

In some examples, the housing comprises at least one channel along the first surface. Thus, the housing may comprise at least one channel in the first surface. For example, there may be a plurality of channels. The channel may extend along the base surface. In particular, the channel may have a depth into the housing (e.g., in the distal direction from the first surface) and a length and width may be arranged along the first surface. The channel may have a proximal side and a distal side. For example, the distal side may be recessed relative to the first surface (e.g., in the distal direction). The first surface may extend generally in a plane, with at least part of the first surface extending in the plane. For example, the first surface may be attached to the adhesive patch for placement on the skin. The proximal side of the channel may be aligned with the plane of the first surface. The opening of the channel on the proximal side may be an opening in the first surface. The channel may provide a region of the housing (e.g., the base) which extends distally and may not be in contact with the adhesive patch. In some examples, the channel may be a groove. The channel may be an open channel. For example, the proximal side of the channel may be open, with the channel defining an interior space. The channel may allow collection of water vapor in through the proximal side (e.g., from the adhesive patch). The water vapor may then be passed along the channel (e.g., along the length of the channel).

In some examples, the at least one channel comprises a first end connected to the vent inlet. For example, the channel may be connected to the vent inlet. This allows water vapor to be collected in the channel and passed to the vent inlet for release from the vent outlet. In some examples, a plurality of channels may be provided, and at least one channel may be connected to the vent inlet (e.g., at least one channel may not be connected to the vent inlet). In some examples, a plurality of vent inlets may be provided, and at least one channel (e.g., a plurality of channels) may be connected to at least one vent inlet (e.g., each vent inlet). For example, a second end of the at least one channel may be connected to a second vent inlet.

In some examples, a second end of the channel may terminate in the first surface at a position spaced away from a periphery. For example, preferably the channel does not extend to the periphery of the first surface (e.g., a periphery of the base surface). This allows the surface area at the periphery of the first surface to be increased, leading to more contact with the adhesive patch and improved adhesive effectiveness. In other examples, a second end of the channel may terminate at the periphery of the first surface. For example, the channel may extend to the periphery of the first surface. This may allow water vapor to be collected by the channel and passed to the periphery of the first surface for release (e.g., from the periphery of the housing).

In some examples, at least one channel is connected to the first aperture in the first surface. For example, the first end of the channel may be connected to the first aperture. This may allow water vapor to be collected in the channel and passed to the first aperture for release from the second aperture in the second surface. In some examples, the first end of the channel may be connected to the vent inlet and a second end may be connected to the first aperture. This may allow water vapor to be collected from the first aperture, passed along the channel, and into the vent inlet for release through the vent outlet.

In some examples, the first surface comprises a plurality of vent inlets. For example, the base surface may comprise a plurality of vent inlets. For example, the base surface may comprise a second vent inlet. In some examples, there may be two or more, such as three or more vent inlets. This can increase the collection of water vapor. There may be a plurality of vent passageways connected to respective vent inlets.

In some examples, the second surface comprises a plurality of vent outlets. For example, the second surface may comprise a second vent inlet. In some examples, there may be two or more, such as three or more vent outlets. This can increase the release of water vapor. There may be a plurality of vent passageways connected to respective vent outlets. In some examples, at least one vent outlet may be provided on different surfaces (e.g., a top surface, a side surface, or an intermediate surface). For example, at least one vent outlet may be provided in the top surface and at least one vent outlet may be provided in the side surface.

In some examples, each of the plurality of vent inlets is fluidly connected to a respective one of the plurality of vent outlets. For example, there is a one-to-one correspondence between the vent inlets and the vent outlets. For example, this can form a plurality of individual vent passageways. Thus, in some examples, each vent passageway may not be connected to other vent passageways.

In some examples, a plurality of vent inlets is fluidly connected to one vent outlet. For example, there is a many-to-one correspondence between the vent inlets and the vent outlets. For example, a group of vent inlets can be connected together, and a shared vent passageway section can extend to the vent outlet. This can increase the collection of water vapor whilst minimizing the impact on space in the housing. For example, the vent inlets can be connected proximally of the sensor electronics, and the shared vent passageway section may pass through the sensor electronics (compared to a plurality of vent passageways). This may also reduce the risk of liquid water entering the vent outlet, since there may be one vent outlet instead of multiple. In some examples, a plurality of vent outlets is fluidly connected to one vent inlet. For example, there is a one-to-many correspondence between the vent inlets and the vent outlets.

In some examples, each of the plurality of vent inlets is fluidly connected to each of the plurality of vent outlets. For example, this can form interconnected vent passageways. This may increase the airflow, improving passage of water vapor.

Disclosed herein is an on-body device for monitoring an analyte level of a user, comprising: a housing, comprising: a first side comprising an aperture and a vent inlet; and a second side comprising a vent outlet; wherein the vent inlet is fluidly connected to the vent outlet through the housing to allow passage of water vapor from the first side of the housing to the second side; an adhesive patch attached to the housing and configured to attach the housing to skin of the user; sensor electronics, wherein the housing is configured to retain the sensor electronics; and an analyte sensor, comprising: a first portion configured to be in electrical contact with the sensor electronics; and a second portion configured to be positioned in the body of the user for monitoring an analyte level of the user; wherein the analyte sensor is configured to extend through the aperture for positioning the second portion of the analyte sensor in the body of the user. The first side may be a proximal side or a base side. The second side may be a distal side or a top side. The adhesive patch may be attached to the first side. The first side may be opposite to the second side. The housing may comprise a third side opposite to the first side (e.g., the third side may be a distal side or a top side), and the second side may be a peripheral side (e.g., arranged between the first side and the third side). Features of the first aspect may be readily applied to this disclosure.

Disclosed herein is an on-body device for monitoring an analyte level of a user, comprising: a housing, comprising: a first surface comprising a vent inlet; and a second surface comprising a vent outlet; wherein the vent inlet is fluidly connected to the vent outlet through the housing to allow passage of water vapor from the first surface to the second surface; an adhesive patch attached to the housing and configured to attach the housing to skin of the user; sensor electronics, wherein the housing is configured to retain the sensor electronics. The on-body device may be configured to communicate with an analyte sensor configured to be positioned in the body of the user for monitoring an analyte level of the user. For example, the analyte sensor may be wholly implantable in the body and may be configured to communicate with the sensor electronics. For example, the analyte sensor may communicate wirelessly with the sensor electronics without being physically connected. Features of the first aspect may be readily applied to this disclosure.

According to another aspect of the present disclosure, there is provided: an analyte monitoring system, comprising: the on-body device for monitoring the analyte level of the user according to the first aspect; and a computer program for execution on a receiver unit, wherein the computer program is configured to control the receiver unit to communicate with the on-body device for receiving analyte data associated with the analyte level of the user and to output information corresponding to the analyte data to the user.

The analyte monitoring system may thus include the on-body device described herein as well as a computer program for a receiver unit. For example, the computer program may be a software application. The computer program can cause the receiver unit to communicate with the on-body device, in particular to receive analyte data. With this data, the computer program can then cause the receiver unit to output information corresponding to the analyte data. For example, the computer program may cause the receiver unit to output a display of the current analyte level of the user. The computer program may be provided by the developer (e.g., the manufacturer of the on-body device) and downloaded by the user from an application store onto the receiver unit. In other examples, the computer program may be pre-installed (e.g., if the developer is the manufacturer of the receiver unit, such as a dedicate reader device for analyte monitoring).

The computer program may be referred to as a computer program product. The computer program (or computer program product) may comprise instructions which, when the program is executed by a computer (e.g., the receiver unit), cause the computer to carry out the functionality described. Disclosed herein is an analyte monitoring system, comprising: the on-body device for monitoring the analyte level of the user according to the first aspect; a computer program comprising instructions which, when the program is executed by a computer (e.g., the receiver unit), cause the computer to communicate with the on-body device for receiving analyte data associated with the analyte level of the user and output information corresponding to the analyte data to the user.

In some examples, the analyte monitoring system further comprises a receiver unit configured to execute the computer program. For example, the receiver unit may be a mobile device, such as a smartphone. In other examples, the receiver unit may be a wearable device such as a smartwatch. In other examples, the receiver unit may be a computing device, such as a computer (e.g., a tablet computer, a laptop computer, or a desktop computer) or a processing unit within a vehicle (e.g., a car or a motorcycle). In other examples, the receiver unit may be a dedicated reader device for analyte monitoring, for example provided by the manufacturer of the on-body device. In other examples, the receiver unit may be connected to or included as part of a drug delivery device, such as an insulin pump or an insulin pen. The analyte monitoring system may include multiple such receiver units.

The analyte monitoring system may include a computer-readable storage medium. The computer-readable storage medium may comprise instructions which, when executed by a computer (e.g., the receiver unit), cause the computer to carry out the functionality described. Disclosed herein is an analyte monitoring system, comprising: the on-body device for monitoring the analyte level of the user according to the first aspect; a computer-readable storage medium comprising instructions which, when executed by a computer (e.g., the receiver unit), cause the computer to communicate with the on-body device for receiving analyte data associated with the analyte level of the user and output information corresponding to the analyte data to the user.

According to another aspect of the present disclosure, there is provided: an insertion system, comprising: the on-body device for monitoring the analyte level of the user according to the first aspect; and an inserter configured to position the on-body device on the skin of the user with the second portion of the analyte sensor positioned in the body of the user.

The insertion system may thus include the on-body device described herein as well as an inserter for inserting/applying the on-body device. Various inserters may be used as described herein. In some examples, the inserter may comprise a sharp. The inserter may be used to insert the sharp into the skin for positioning the second portion of the analyte sensor in the body of the user. In other examples, the analyte sensor may be configured to pierce the skin without a sharp. In such examples, the inserter may be configured to insert the analyte sensor into the skin without a sharp.

In some examples, the inserter may be configured to advance the on-body device within the inserter. For example, the housing may be advanced within the inserter. For example, the on-body device may be provided within the inserter. In particular, the inserter may move the on-body device towards the skin of the user, thereby inserting the analyte sensor (and optionally the sharp) into the body. For example, the analyte sensor may be inserted such that the second portion is in contact with interstitial fluid in the body of the user. When the analyte sensor is inserted into the body, the on-body device can be on the skin, with the adhesive patch adhered to the skin.

The inserter may comprise an insertion mechanism for inserting the analyte sensor (and optionally advancing the housing). For example, the inserter may be configured to automatically insert the analyte sensor. For example, the insertion mechanism may comprise an insertion spring. In other examples, the analyte sensor may be inserted (and the housing may optionally be advanced) by applying a force to a movable handle.

In other examples, the housing may be placed on the skin (e.g., at the bottom of the inserter) and the inserter may be used to insert the analyte sensor into the skin (e.g., through the housing, for example coupling the analyte sensor to the sensor electronics). In some examples, the analyte sensor may be pre-assembled with the housing (e.g., during manufacture) and may be provided within the inserter. In other examples, the analyte sensor may be assembled with the housing (e.g., attached to the sensor electronics) before or during the process of inserting the analyte sensor.

In some examples, the inserter may be configured to retract the sharp from the skin after insertion. For example, the inserter may be configured to automatically retract the sharp.

The inserter may comprise a retraction mechanism for retracting the sharp. For example, the retraction mechanism may comprise a retraction spring.

In some examples, the inserter comprises a cap. The cap may be attached to an end of the inserter, for example by threads. The cap may maintain a sterile environment inside the inserter and may protect the user from the sharp.

According to another aspect of the present disclosure, there is provided: an on-body device for monitoring an analyte level of a user, comprising: a housing, comprising: a first surface comprising a vent inlet; and a second surface comprising a vent outlet; wherein the vent inlet is fluidly connected to the vent outlet through the housing to allow passage of water vapor from the first surface to the second surface; an adhesive patch attached to the housing and configured to attach the housing to skin of the user; and electronics configured to receive a signal from a sensor configured to measure a level of an analyte of the user, wherein the signal comprises information indicative of a level of an analyte of the user and wherein the housing is configured to retain the electronics, wherein the on-body device is not configured to operate with a partially in vivo sensor.

The on-body device can be configured similar to all the variations described with respect to the first aspect not related to the partially implantable sensor. Methods of use and insertion systems are also provided according to the fourth aspect.

According to another aspect of the present disclosure, there is provided: an on-body device for monitoring an analyte level of a user, comprising: a housing comprising at least one passageway through the housing; a moisture permeable seal configured to allow passage of water vapor through the passageway; sensor electronics, where the housing is configured to retain the sensor electronics; and an analyte sensor configured to monitor an analyte level of the user and configured to be in communication with the sensor electronics.

In some examples, the moisture permeable seal is at least partially within the passageway. In other examples, the moisture permeable seal is located entirely within the passageway. The housing may comprise a lower base surface and an upper surface, where the passageway extends between an opening in the lower base surface and an opening in the upper surface. The moisture permeable seal can be located at an upper location, an intermediate location, or a lower location within the passageway.

In some examples, the moisture permeable seal is a first moisture permeable seal, and the on-body device further comprises a second moisture permeable seal configured to allow passage of water vapor through the passageway. The first moisture permeable seal may be located at an upper location within the passageway and the second moisture permeable seal may be located at a lower location within the passageway. The on-body device can further comprise a third moisture permeable seal configured to allow passage of water vapor through the passageway, where the third moisture permeable seal can be located at an intermediate location within the passageway.

In some examples the moisture permeable seal has a thickness that is 50% or less of a length of the passageway. The moisture permeable seal can have a thickness that is 33% or less of a length of the passageway. The moisture permeable seal can have a thickness that is 25% or less of a length of the passageway.

In some examples the housing comprises a recess in a wall of the passageway, where at least a portion of the moisture permeable seal is in the recess. The recess can be annular.

In some examples the housing comprises a projection from a wall of the passageway into an interior of the passageway, where at least a portion of the moisture permeable seal abuts the projection. The projection can be annular.

In various examples the moisture permeable seal comprises a flange and a columnar portion. The flange and columnar portion can be located within the passageway, such that an end surface of the columnar portion is flush with a surface of the housing. The moisture permeable seal can further comprise a head portion configured to reside outside of the passageway and abut a surface of the housing.

In some examples the housing comprises a lower base surface and an upper surface, wherein the passageway extends between an opening in the lower base surface and an opening in the upper surface, and wherein the moisture permeable seal is configured as a cap for the opening in the upper surface. The cap can comprise a roof member and a sidewall. The sidewall can comprise a first retention feature. The first retention feature can be configured to interlock with a second retention feature in a receiving space within the housing. The cap can be configured to be manually attached to the housing by a user. The cap can comprise an inner passageway for passage of a sharp therethrough.

In many examples the passageway is a sensor passageway. The passageway can extend between a first aperture in an upper surface of the housing and a second aperture in a base surface of the housing, where the analyte sensor projects from the housing through the second aperture.

In many examples the passageway is a vent passageway. The passageway can extend between a vent inlet (e.g., an opening) in an upper surface of the housing and a vent outlet (e.g., an opening) in a base surface of the housing. The passageway can be a vent passageway and the moisture permeable seal can be a first moisture permeable seal, where the housing also comprises a sensor passageway, and where the on-body device comprises a second moisture permeable seal configured to allow the passage of water vapor through the sensor passageway. The on-body device can further comprise a third passageway configured as a vent passageway, where the on-body device comprises a third moisture permeable seal configured to allow the passage of water vapor through the third passageway. The passageway or vent passageway can be a vent compartment.

In some examples, the moisture permeable seal comprises an adhesive and is configured to adhere to an upper surface of the housing, where the upper surface is opposite a lower base surface of the housing. The moisture permeable seal can be configured to cover an opening to the passageway in the upper surface of the housing. The housing can comprise a plurality of passageways, including at least one vent passageway and a sensor passageway, and the moisture permeable seal can be configured to cover openings to each of the plurality of passageways. The moisture permeable seal can be configured as a sticker.

In many examples the moisture permeable seal comprises an inner passageway configured to permit passage of a sharp therethrough. The moisture permeable seal can comprise an elastic material biased to self-close the inner passageway.

In many examples the on-body device further comprises an adhesive patch coupled with a base surface of the housing. The moisture permeable seal can comprise a columnar portion configured to reside within the passageway and a head portion configured to abut the base surface of the housing. The adhesive patch can hold the moisture permeable seal against the base surface.

In many examples, the moisture permeable seal comprises a moisture permeable material. The moisture permeable seal can comprise silicone. The moisture permeable material can comprise a fibrous material. The moisture permeable material can comprise poly(methyl methacrylate).

In many examples, the moisture permeable seal is configured to allow passage of water vapor therethrough and block passage of liquid water through the passageway.

In many examples, the analyte sensor comprises: a first portion configured to be in electrical contact with the sensor electronics; and a second portion configured to be positioned in the body of the user for monitoring an analyte level of the user, wherein the analyte sensor is configured to extend through an aperture for positioning the second portion of the analyte sensor in the body of the user.

According to another aspect of the present disclosure, there is provided: a method of manufacturing an on-body device for monitoring an analyte level of a user, comprising: providing a housing comprising at least one passageway through the housing; and injecting a moisture permeable material into the passageway, wherein the moisture permeable material forms a seal configured to allow passage of water vapor through the passageway. The method can further comprise: coupling sensor electronics with the housing; and coupling an analyte sensor to the sensor electronics, wherein the analyte sensor is configured to monitor an analyte level of the user.

According to another aspect of the present disclosure, there is provided: a method of manufacturing an on-body device for monitoring an analyte level of a user, comprising: providing a housing comprising at least one passageway through the housing; and inserting a moisture permeable seal into the passageway or into a position that covers the passageway, wherein the moisture permeable seal is configured to allow passage of water vapor through the passageway. The method can further comprise: coupling sensor electronics with the housing; and coupling an analyte sensor to the sensor electronics, wherein the analyte sensor is configured to monitor an analyte level of the user.

According to another aspect of the present disclosure, solid foam seals are provided that can be used with any and all embodiments described herein, including all aspects of the present disclosure described here.

According to a another aspect of the present disclosure, fibrous seals are provided that can be used with any and all embodiments described herein, including all aspects of the present disclosure described here.

Aspects described here relate to an on-body device comprising: electronics configured to receive a signal from a sensor, the signal indicative of a level of an analyte of a user; and a housing configured to retain the electronics, and wherein the housing may be adapted to permit egress of moisture from an area adjacent the housing.

The on-body device may include, or be combined with, any of the features of the on-body device according to the aforementioned aspects. Features referred to with the same terminology are the same, or corresponding, features.

Advantageously, the housing being adapted to permit moisture egress can reduce moisture buildup under the device, thereby improving skin health and reducing the shedding of dead skin cells, and enhancing adhesive performance and reducing the likelihood of adhesive patch peel away. Ultimately, this allows for longer device wear times.

The housing may comprise a base having a first surface for facing a skin surface of the user.

The base may comprise a first region, and the first region may comprise an aperture in the housing.

Such an aperture may accommodate components such as a sensor and sharp.

The first region of the base may be spaced apart from a periphery of the base.

The aperture may be positioned in the center of the first region.

Such configurations of the first region and aperture permit ease of operation with insertion devices.

The first region may comprise a recessed region of the base.

A recessed region can protect components associated with the aperture and permit connection with a sharp cap for protecting the sharp.

The housing may comprise at least one channel along the first surface, wherein the at least one channel may be configured to permit egress of moisture from an area adjacent the first surface.

Advantageously, providing channels creates defined pathways for moisture (liquid or vapor) to move away from the skin that is beneath the on-body device, thereby reducing moisture accumulation, enhancing skin breathability, and thus increasing delamination time of an adhesive patch.

The at least one channel may be formed in the base of the housing.

The at least one channel may comprise one or more first channels and/or one or more second channels.

Different types of channels allow for complex and optimized patterns tailored to specific moisture egress needs and/or device geometries.

Each of the one or more first channels may comprise a first end arranged at a periphery of the base.

Having channels extending to the periphery provides a direct exit route for moisture to escape from under the device.

At least a portion of the one or more first channels may comprise a second end arranged at the first region of the base.

Channels connecting the periphery to a central region can allow moisture egress away from critical areas, such as a sensor insertion site located in the first region.

Each of the one or more second channels may comprise a first end arranged at the periphery of the base and a second end arranged at the periphery of the base.

Such channels can provide multiple pathways by which moisture can exit at the periphery of the on-body device. As such, if one pathway becomes blocked (e.g. due to skin cells or liquid droplets), alternative exit pathways for moisture are provided.

Each of the one or more second channels may comprise a curved shape.

Such configurations can optimize flow paths around central features of the on-body device, and create parallel pathways.

Each of the one or more second channels may be arranged to intersect at least one of the first channels.

Intersecting channels create a network, enhancing the distribution and collection of moisture across the base surface and providing multiple egress pathways.

The depth of each of the one or more first channels and/or each of the one or more second channels may vary along at least a portion of the length of the respective channel.

The depth of each of the one or more first channels may decrease as each respective first channel progresses toward the first region of the base.

Advantageously, this configuration avoids the creation of an abrupt recessed space at the first region that could lead to induration of the skin. Such configurations can also promote the directing of moisture away from the first region and toward the periphery, and can minimise any compromises to structural integrity resulting from the channels.

The depth of each of the one or more first channels may decrease to zero as the respective first channel progresses toward the first region of the base.

Tapering channels to zero depth allows them to smoothly merge with the base surface and first region. A gradual and continuous depth variation avoids the creation of an abrupt edge or recess that could lead to induration of the skin.

The depth of each of the one or more first channels may decrease as each respective channel progresses toward the first region of the base until each respective first channel bottom meets the first surface of the base.

Depth variation of this type provides a smooth transition from the channel bottom to the surrounding base surface.

The portion of the length of the respective channel may be 1% or greater, 2% or greater, 3% or greater, 5% or greater, 8% or greater, 10% or greater, 15% or greater, 20% or greater, 25% or greater, 30% or greater, 50% or greater, 75% or greater, or 95% or greater of the length of the respective channel.

Each of the one or more first channels and/or each of the one or more second channels may taper in depth.

Each of the one or more first channels may include a tapered edge and/or a bevelled edge where the respective channel meets the first region.

Smooth transitions in depth provided by the tapered and/or bevelled edge reduce the risk of induration of the skin. A smooth transition can also reduce stress concentrations and improve ease of manufacture.

The depth of each of the one or more first channels and/or each of the one or more second channels may vary in a stepped fashion.

The depth of each of the one or more first channels and/or each of the one or more second channels may decrease with a radiused sloping edge.

A radiused sloping edge provides a smooth, curved transition in depth, which reduces the risk of induration of the skin.

A depth of each of the one or more first channels and/or each of the one or more second channels may increase as the channel progresses towards the periphery of the base.

Increasing depth towards the periphery can enhance drainage capacity near the exit points.

Each of the one or more first channels and/or each of the one or more second channels may have a maximum depth at or adjacent to the periphery of the base.

Locating the maximum depth at the periphery maximizes the capacity for the exit of moisture.

The width of each of the one or more first channels and/or each of the one or more second channels may taper as the respective channel progresses towards a periphery of the base.

Advantageously, a narrowing or tapering width maximizes surface area at the periphery of the base of the housing for coupling with an adhesive patch, and ultimately with the user. This maximises adhesion surface area at the periphery, to reduce the risk of peeling commencing from the periphery. This arrangement also improves structural integrity of the housing at the edge thereof.

The width of each of the one or more first channels and/or each of the one or more second channels may narrow as the respective channel progresses towards a periphery of the housing.

The width of each of the one or more first channels and/or each of the one or more second channels may taper along a portion of the length of the respective channel at or adjacent a periphery of the base.

Each of the one or more first channels and/or each of the one or more second channels may terminate along a peripheral edge of the housing in the shape of a slit.

A slit termination provides a defined, narrow exit point for moisture while maximising surface area of the base in the periphery, and also prevents moisture from entering, or re-entering, the respective channel. The slit termination may be 5-40% of the width of the main portion of the channel. More preferably, the slit termination may be 10-30% of the width of the main portion of the channel.

Each of the one or more first channels and/or each of the one or more second channels may have a first width at a first location and a second width at a second location, and the first width may transition to the second width in a stepped or continuous fashion.

Allowing for different widths along the channel enables improved and tailored moisture removal efficiency. A wider channel permits greater moisture removal rates, but reduces surface area of the base available for adhesion. Thus, providing for varying widths allows for moisture removal and adhesion to be optimised.

A first width of each of the one or more first channels and/or each of the one or more second channels at the first location may be less than a second width of the respective channel at the second location.

A first depth of each of the one or more first channels and/or each of the one or more second channels at the first location may be less than a second depth of the channel at the second location.

Each of the one or more first channels and/or each of the one or more second channels may have a first lengthwise span adjacent a periphery of the base and a second lengthwise span located relatively farther from the periphery of the base than the first lengthwise span.

The first lengthwise span may maintain the first width in constant fashion and the second lengthwise span may maintain the second width in constant fashion.

The first lengthwise span may vary in width across a range that includes the first width, and the second lengthwise span may maintain the second width in constant fashion.

The first lengthwise span may maintain the first width in constant fashion, and the second lengthwise span may vary in width across a range that includes the second width.

The first lengthwise span may vary in width across a range that includes the first width, and the second lengthwise span may vary in width across a range that includes the second width.

The first lengthwise span may gradually transition in width to the second lengthwise span.

A gradual transition avoids abrupt changes that could affect flow or manufacturability.

The first lengthwise span and the second lengthwise span of each of the one or more first channels may be separated due to intersection with one or more of the second channels.

Each of the one or more first channels and/or each of the one or more second channels may have a non-constant width along an entire length thereof.

A width of each of the one or more second channels may be constant along its entire length.

Each of the at least one channels may comprise a first sidewall, a second sidewall, and a channel bottom that is distal to the first surface.

The first sidewall and the second sidewall may extend between the first surface and the channel bottom.

The depth of each of the at least one channels may be a distance between the respective channel bottom and the first surface.

The depth of each of the at least one channels may be a distance between the respective channel bottom and the first surface of the base along an axis normal to a plane of the first surface of the base.

The first sidewall may meet the first surface at a first intersection, the second sidewall may meet the first surface at a second intersection directly opposite the first intersection, and the width of the channel may be a distance between the first intersection and the second intersection.

The channel may comprise a fillet or a chamfer at the first intersection and/or the second intersection.

Fillets or chamfers can reduce stress concentrations and improve flow characteristics at the channel edges.

The base may comprise an interior surface region and a peripheral surface region located between the interior region and the periphery.

Each of the one or more first channels may extend across the base in both the interior surface region and the peripheral surface region, wherein each of the one or more first channels has a narrower width in the peripheral surface region than in the interior surface region.

A first subset of the one or more second channels may be closer to the periphery of the base than a second subset of the one or more second channels, and the first subset may have a narrower width than the second subset.

The one or more second channels may comprise an arc.

Arcuate channels can efficiently follow the perimeter of a circular or rounded device, and provide smooth, interconnected and parallelised exit pathways for moisture.

The curved channel or arc may have two ends that terminate at the periphery of the base. The arc may be greater than 90 degrees and less than 360 degrees. The arc may be greater than 180 degrees and less than 360 degrees. The arc may be greater than 210 degrees and less than 360 degrees.

The arc may be an arc of a circle or an ellipse.

The curved shape may have a constant radius of curvature.

The curved shape may be C-shaped.

The curved shape may be U-shaped.

The curved shape may comprise a first curved portion and a second curved portion.

The first curved portion may comprise a first focal point and the second curved portion may comprise a second focal point.

The first curved portion may have a first radius of curvature and the second curved portion may have a second radius of curvature.

The one or more second channels may not comprise any corner that has an angle of less than 135°.

The one or more second channels may not comprise any bend that has a bend radius of less than 2 mm.

The curved shape of at least some of the one or more second channels may comprise an interior side of the curve arranged towards the first region of the base.

A focal point of the curved shape of the at least some of the one or more second channels may be the aperture.

The aperture may be arranged off-centre in the base of the housing.

The aperture may be arranged on a first side of the first surface of the base relative to a centre of the base, and at least a portion of the one or more second channels may be arranged on a second side of the first surface of the base opposite to the first side of the first surface of the base relative to the centre of the base.

The aperture in the base of the housing may define a sensor passageway for receiving the sensor and/or for receiving a sharp for positioning the sensor.

The base may further comprise one or more third channels comprising a first end arranged at the periphery of the base and extending to a second end connected to the at least one second channel.

Advantageously, the third channel can connect the periphery to the arcuate second channels, further enhancing the network and exit pathways for moisture.

The one or more first channels may be straight channels.

Each of the one or more first channels may extend radially from a position on the first surface of the base that is offset from the periphery of the base.

The position may correspond to the position of the aperture in the housing.

The one or more first channels may extend radially between a periphery of the base and an interior region of the base.

This arrangement promotes moisture drainage from the interior to the exit points at the edge.

Each of the one or more first channels and/or each of the one or more second channels may have a cross-sectional profile that is U-shaped, V-shaped, or three-sided.

Each of the one or more first channels and/or each of the one or more second channels may have a three-sided cross-sectional profile with two interior right angles.

Each of the one or more first channels and/or each of the one or more second channels may have a three-sided cross-sectional profile with two interior angles between 45 and 90 degrees.

This defines a trapezoidal cross-section with angled sidewalls.

Each of the one or more first channels and/or each of the one or more second channels may have a three-sided cross-sectional profile with two interior angles between 60 and 90 degrees.

Each of the one or more first channels and/or each of the one or more second channels may have a three-sided cross-sectional profile with a fillet or a chamfer at a side intersection.

Each of the one or more first channels and/or each of the one or more second channels may have a maximum depth that is greater than 50 μm.

Each of the one or more first channels and/or each of the one or more second channels may have a maximum depth between 100 μm and 400 μm.

Each of the one or more first channels and/or each of the one or more second channels may have a maximum depth between 100 μm and 200 μm.

Each of the one or more first channels and/or each of the one or more second channels may have a maximum depth of around 100 μm.

The term “around” may indicate a tolerance (e.g. a manufacturing tolerance), such as ±25%, ±10%, ±5%, ±2%, or ±1%. For instance, the maximum depth may be 100 μm±25%, or 100 μm±10%, or 100 μm±5%.

A relatively shallow channel, around 100 μm, ensures that the depth or height of the housing does not need to be excessively large so as to accommodate the depth of the channels therein. Whilst a deeper channel can promote greater moisture egress, this must be balanced against an increase in required device height/depth to accommodate such channels. A shallow channel can also improve adhesion, as a portion of an adhesive patch may flex inwards and contact a portion of the base of a channel. A wide channel can be selected to promote greater moisture removal rates, more than compensating for any loss of moisture removal rates resulting from a shallow channel.

Each of the one or more first channels and/or each of the one or more second channels may have a width between 400 μm and 1000 μm.

Each of the one or more first channels and/or each of the one or more second channels may have a width between 600 μm and 900 μm.

Each of the one or more first channels and/or each of the one or more second channels may have a width between 700 μm and 800 μm.

Each of the one or more first channels and/or each of the one or more second channels may have a width of around 800 μm.

A wide channel, of around 800 μm, can enhance moisture removal rates without requiring a larger device housing to accommodate the channels. Adequate surface area must remain between the channels to permit adhesion.

Each of the one or more first channels and/or each of the one or more second channels may have a width of around 800 μm and a maximum depth of around 100 μm.

The combination of a wide and shallow channel, of around 800 μm wide and 100 μm deep, allows for unexpectedly high moisture removal rates, without requiring an undesirably deep/tall housing to accommodate the channels.

Each of the one or more first channels and/or each of the one or more second channels may have a width to maximum depth ratio of around 8:1.

This ratio has been found to provide a beneficial balance between moisture removal rates, device/housing height to accommodate the channels, and adhesion strength/wear duration.

Each of the one or more first channels and/or each of the one or more second channels may have a rectangular cross-section.

Advantageously, a rectangular cross-section provides the maximum volume within the channels for moisture removal.

The maximum depth may be measured between the bottom of the respective channel and first surface of the base along a direction normal to the first surface of the base.

The width of the channel may be measured between opposing sidewalls of the respective channel, in the plane of the first surface of the base and normal to the lengthways axis of the respective channel.

The first surface of the base may comprise a plurality of proximal portions between the one or more first channels and/or the one or more second channels.

The term “proximal”, as referred to herein, refers to the side of the on-body device that is closest to the skin of the user in use, i.e. the bottom or lower surface of the on-body device.

The plurality of proximal portions may define a proximal surface of the base of the housing.

Such proximal surface provides a skin contacting surface or an adhesive contacting surface.

The plurality of proximal portions may comprise protrusions of the base to define boundaries of the one or more first channels and/or the one or more second channels.

The proximal portions may be integrally formed with the base of the housing.

The proximal portions may define a contact surface area of between 30% and 80% of the proximal surface of the base.

The proximal portions may define a contact surface area of between 50% and 60% of the proximal surface of the base.

The proximal portions may define a contact surface area of around 55% of the proximal surface of the base.

The proximal portions may comprise a first proximal portion and a second proximal portion each having a contact surface for contact with the adhesive patch, wherein the first proximal portion and the second portion are defined, at least in part, by channels of the one or more first channels and/or the one or more second channels, and wherein the contact surface of the first proximal portion is 1.25 times as large or more in surface area than the contact surface of the second proximal portion.

The contact surface of the first proximal portion may be two times as large or more in surface area than the contact surface of the second proximal portion.

The contact surface of the first proximal portion may be three times as large or more in surface area than the contact surface of the second proximal portion.

The first proximal portion may not extend to the peripheral edge of the base.

The first proximal portion may have a contact surface with a surface area that is 5% or greater of the proximal surface of the base.

The first proximal portion may have a contact surface with a surface area that is 7% or greater of the proximal surface of the base.

The first proximal portion may a contact surface with a surface area that is 10% or greater of the proximal surface of the base.

The proximal portions may comprise a moisture-permeable material.

The one or more first channels and/or the one or more second channels may comprise grooves formed in the base of the housing.

The housing may comprise: at least one vent inlet in the first surface; and at least one vent outlet in a second surface of the housing, the second surface opposing the first surface, wherein the vent inlet may be fluidically connected to the vent outlet through the housing to allow the passage of water vapor from the first surface to the second surface.

Advantageously, such vent inlets provide an additional or alternative pathway for moisture egress.

The at least one vent inlet may be spaced away from a periphery of the first surface.

Locating the vent inlet internally addresses moisture trapped away from the edges.

The at least one vent outlet may be aligned with the at least one vent inlet along an axis perpendicular to the first surface.

The at least one vent outlet may be smaller than the at least one vent inlet.

This may help prevent ingress of external liquid while allowing vapor out.

The at least one vent outlet may comprise a width of less than 1 mm.

The housing may comprise a vent passageway between the at least one vent inlet and the respective at least one vent outlet.

Each vent passageway may have a longitudinal axis that is normal to at least a portion of the first surface at the respective vent inlet.

Each vent passageway may have a longitudinal axis that is angled with respect to at least a portion of the first surface at the respective vent inlet.

The longitudinal axis of each vent passageway may be angled from the respective vent inlet to the respective vent outlet in a direction towards a periphery of the housing.

The longitudinal axis of each vent passageway may be angled from the respective vent inlet to the respective vent outlet in a direction away from a periphery of the housing.

Each of the at least one vent inlets may be wider than the respective at least one vent outlet, and the respective vent passageway may comprise a tapered width portion.

Each of the at least one vent outlets may be wider than the respective vent inlet, and the respective vent passageway may comprise a tapered width portion.

Each of the at least one vent inlets may be wider than the respective vent outlet, and the respective vent passageway may comprise a step transition in width from the vent inlet to the vent outlet.

Each vent passageway may have a first portion that extends along a first longitudinal axis, and a second portion that extends along a second longitudinal axis, wherein the first longitudinal axis may be transverse to the second longitudinal axis.

The first longitudinal axis may be perpendicular to the second longitudinal axis.

Each vent passageway may comprise a portion that extends along a curved longitudinal axis.

Each entire vent passageway may extend along a curved longitudinal axis.

Each of the at least one vent inlets may be relatively farther from the periphery than the respective vent outlet.

Each of the at least one vent inlets may be relatively closer to the periphery than the respective vent outlet.

Each vent passageway may extend through the electronics.

Each vent passageway may extend through an aperture in a printed circuit board of the electronics.

The at least one channel may comprise one or more fourth channels connected to the at least one vent inlet.

Advantageously, this integrates the channels in the base of the housing with through-housing vents, providing a combined moisture management system, enabling moisture egress from across the base surface and through the vents. It also avoids a need for moisture outlets at the periphery of the housing, ensuring that the periphery of the base surface of the housing can be an uninterrupted smooth surface, provided maximal surface area for adhesion in the peripheral region where peel risk is greatest.

The one or more fourth channels may terminate at or adjacent to the respective vent inlet.

The one or more fourth channels may be fluidically connected to the at least one vent inlet.

The at least one vent inlet may comprise a plurality of vent inlets, and each of the one or more fourth channels may comprise a first end arranged at a first vent inlet of the plurality of vent inlets and a second end arranged at a second vent inlet.

Advantageously, this allows channels to interconnect multiple vent inlets, creating a network feeding moisture to the vents for removal.

The plurality of vent inlets may comprise a first, a second and a third vent inlet, wherein a first of the one or more fourth channels may be connected to the first and second vent inlets, a second of the one or more fourth channels may be connected to the second and third vent inlets, and a third of the one or more fourth channels may be connected to the first and third vent inlets.

This arrangement provides an approximately triangular arrangement of channels and vent inlets.

Each of the one or more fourth channels may be spaced away from a periphery of the base of the housing.

The at least one vent inlet may comprise a plurality of vent inlets, and a fourth channel of the one or more fourth channels may comprise a first end arranged at a first vent inlet of the plurality of vent inlets and a second end arranged at the first region of the base.

Another of the one or more fourth channels may comprise a first end arranged at a second vent inlet of the plurality of vent inlets, and a second end arranged at the first region.

Another of the one or more fourth channels may comprise a first end arranged at the first vent inlet and a second end arranged at a third vent inlet of the plurality of vent inlets.

Another of the one or more fourth channels may comprise a first end arranged at the second vent inlet and a second end arranged at the third vent inlet.

Each of the at least one vent inlets may be formed in a bottom of a respective fourth channel.

The one or more fourth channels may be narrower than the vent inlets.

The one or more fourth channels may be wider than the vent inlets.

The one or more fourth channels, the plurality of vent inlets and/or the first region may be arranged in a shape, wherein the shape may comprise: a line; a circle; an ellipse; an oval; a semi-circle; a triangle; a square; a pentagon; a hexagon; a heptagon; an octagon; or a star.

The vent inlets may be positioned at vertices or corners of the respective shape.

The vent inlets may be positioned along edges of the respective shape, the edges being defined by the fourth channels.

The one or more fourth channels may have a maximum depth between 100 μm and 400 μm.

The one or more fourth channels may have a maximum depth of around 100 μm.

The one or more fourth channels may have a width between 400 μm and 1000 μm.

The one or more fourth channels may have a width of around 800 μm.

The one or more fourth channels may have a U-shaped, V-shaped or three-sided cross-sectional shape.

The one or more fourth channels may have a curved shape.

The one or more fourth channels may be straight.

The one or more fourth channels may comprise a tapered edge, a beveled edge and/or a radiused sloping edge where the respective fourth channel connects to the respective vent aperture.

The one or more fourth channels may comprise a tapering or narrowing depth as the fourth channel progresses toward the vent aperture.

The one or more fourth channels may comprise a tapering or narrowing width along portions of the fourth channel that are closer to a periphery of the base of the housing.

The device may further comprise a moisture permeable seal provided in each vent passageway.

Advantageously, incorporating seals within vent passageways can control the type of moisture transport (e.g., vapor only) and/or prevent ingress of external liquids/contaminants.

The first surface of the base may comprise a surface texture.

Surface texture can enhance adhesion of an adhesive patch to the base of the housing by effectively increasing surface area and providing mechanical interlocking features. Such features can strengthen the adhesive bond.

The surface texture may facilitate the movement of moisture or liquid across a surface of the base.

Texture can create pathways to promote moisture spreading or channeling, aiding evaporation and/or movement towards egress features.

The device may further comprise a flexible patch adhesively coupled with the base, wherein the surface texture may facilitate adhesive coupling between the base and the flexible patch.

The surface texture may be substantially filled with adhesive.

Filling the texture with adhesive maximizes the contact area and mechanical interlocking for improved adhesion.

The at least one channel may be substantially not filled with adhesive.

Keeping channels clear ensures they remain open pathways for moisture transport, complementing the adhesive enhancement provided by the texture.

The surface texture may comprise directional surface texture.

Directional texture provides anisotropy, allowing for a preferential moisture flow direction and/or resistance to shear forces in specific orientations.

At least a portion of the surface texture may be oriented to facilitate movement of moisture or liquid towards a periphery of the base.

Directing moisture towards the edges via texture aids in moisture removal from under the device.

The surface texture may have an average width and an average depth.

The average width of the surface texture may be greater than the average depth of the surface texture.

The average width of the surface texture may be two or more times the average depth of the surface texture.

The average width of the surface texture may be three or more times the average depth of the surface texture.

The surface texture may have an average pitch that is three or more times the average depth of the surface texture.

The surface texture may comprise a plurality of surface grooves.

Each one of the plurality of surface grooves may have a cross-sectional profile that is U-shaped, V-shaped, or three-sided.

Each of the plurality of surface grooves may have a depth of between 42 and 57 μm, a width of between 420 and 570 μm, and a pitch of between 680 and 920 μm.

Each of the plurality of surface grooves may have a depth of around 50 μm, a width of around 600 μm, and a pitch of around 850 μm.

Each of the plurality of surface grooves may have a depth of between 21 and 29 μm, a width of between 150 and 200 μm, and a pitch of between 340 and 460 μm.

Each of the plurality of surface grooves may have a depth of around 40 μm, a width of around 100 μm, and a pitch of around 300 μm.

At least a portion of the surface texture may be on an area of the base comprising adhesive.

The surface texture may be oriented within 15 degrees of a common direction.

The surface texture may be oriented in substantially the same direction.

A parallel surface texture arrangement may permit controlled flow of moisture towards egress features, such as outlets, vent outlets, and/or channels.

The directionality of the surface texture may be oriented straight.

The directionality of the surface texture may follow a curved direction.

The directionality of the surface texture may be circular.

The directionality of the surface texture may be elliptical, triangular or square.

The directionality of the surface texture may follow the outline of a trapeze, a truncated circle, an oval, an ellipse, a pentagon, a hexagon, a heptagon, or an octagon.

The directional surface texture may be applied to the base of the housing in a concentric pattern.

Such patterns may ensure that the surface texture runs approximately perpendicular to radial channels, to direct moisture into radial channels for removal.

A centroid or focus of the concentric pattern may correspond with the centroid of the shape of the periphery of the base.

A centroid or focus of the concentric pattern may correspond with the location of the aperture in the housing.

Advantageously, centering the surface texture pattern relative to the shape of the base or the sensor aperture may ensure that the surface texture runs approximately perpendicular to radial channels, to direct moisture into radial channels for removal.

The directionality of the surface texture may align with the shape of the periphery of the base.

The surface texture may be oriented so as to be continuously parallel with the peripheral edge of the base.

The surface texture may be oriented so as to be normal to the periphery of the base.

Such an arrangements or patterns of surface texture may allow the surface texture to provide direct pathways to moisture for exiting at the periphery of the device.

The surface texture may be oriented directionally perpendicular to a long axis of the at least one channel.

The surface texture may intersect the at least one channel.

The surface texture may terminate in proximity with the channel.

Consequently, surface texture may lead moisture towards the channels.

The surface texture may be a first surface texture and may be in a first area of the base, and the base may comprise a second surface texture in a second area of the base, wherein a directional orientation of the first surface texture may be different from a directional orientation of the second surface texture.

This allows for multiple zones with different texture orientations on the same base, to achieve different moisture egress and adhesion properties.

The first surface texture may be directionally transverse to a first portion of the at least one channel having a long axis extending in a first direction, and the second surface texture may be directionally transverse to a second portion of the channel having a long axis extending in a second direction different from the first direction.

The first surface texture may be directionally transverse to a first long axis of the one or more first channels, and the second surface texture may be directionally transverse to a second long axis of the one or more second channels, wherein the first long axis and second long axis may be non-parallel.

Such an arrangement provides for different surface texture orientations relative to different types of channels (e.g., radial vs. arcuate), to provide for an enhanced network of moisture removal pathways.

The first surface texture may be directionally straight and the second surface texture may be directionally curved.

The at least one channel may have an average depth that is greater than the average depth of the surface texture.

As such, the surface texture may be considered a small scale surface finish, whereas the channels are deeper grooves within the surface, on a more easily visible scale.

The channel average depth may be at least 10 μm greater than the surface texture average depth.

The channel average depth may be at least 50 μm greater than the surface texture average depth.

The channel average depth may be at least 100 μm greater than the surface texture average depth.

The channel average depth may be at least 150 μm greater than the surface texture average depth.

At least a portion of the surface texture may be oriented to facilitate movement of moisture or liquid towards the at least one channel.

As such, as well as enhancing adhesion, the surface texture has the synergistic advantage of feeding moisture into the channels.

The at least one channel may comprise a channel surface, and at least a portion of the surface texture may be on the channel surface.

As such, the surface texture may be applied even within the channels themselves. This can provide improved routing of moisture within the channels themselves. Such texture can also enhance adhesion, if the channels are sufficiently shallow such that a portion of the adhesive patch will flex and contact the base of the channels.

The at least a portion of the surface texture on the channel surface may be configured to facilitate movement of moisture along the channel.

A base surface adjacent the channel may comprise directional surface texture oriented transverse to a long axis of the at least one channel.

The surface texture may be configured to guide moisture to the at least one channel, and the at least one channel may be configured to guide the moisture to a periphery of the base and/or an end of the respective at least one channel.

As such, there is a cooperative function between the surface texture and channels for moisture egress.

The surface texture may have an arithmetic mean height roughness (Sa) of between 9 and 18 μm, or between 20 and 22 μm.

The surface texture may comprise, at least in part, non-directional surface texture.

Such surface texture may be isotropic. This provides surface roughness that increases the effective surface area and provides mechanical features to enhance adhesion.

The surface texture may have an average roughness (Ra) of at least 0.35 μm.

The surface texture may have an average roughness (Ra) of between 0.56 μm and 0.80 μm.

The surface texture may have an average roughness (Ra) of between 1.12 μm and 1.60 μm.

The surface texture may have an average roughness (Ra) of around 1.5 μm.

The surface texture may have an average roughness (Ra) of between 0.35 μm and 2.24 μm.

The at least one channel may have a first depth that is greater than an average depth of the surface texture.

The channel average depth may be at least 10 μm greater than the surface texture average depth.

The channel average depth may be at least 50 μm greater than the surface texture average depth.

The channel average depth may be at least 100 μm greater than the surface texture average depth.

The channel average depth may be at least 150 μm greater than the surface texture average depth.

The surface texture may be on an area of the base comprising adhesive.

Non-directional texture (roughening) applied to adhesive areas can advantageously improve bond strength.

The surface texture may comprise both non-directional and directional surface texture.

This allows for combining the benefits of both types of texture, e.g., roughening for adhesion and directional texture for moisture guidance.

The device may further comprise a sensor.

The sensor may comprise a sensor body configured to extend through the aperture in the housing and proximally from the base.

The sensor may be an analyte sensor.

The sensor may be a wholly in vivo sensor or a wholly ex vivo sensor.

The sensor may be configured to be implanted in its entirety in the body of the user such that no part of the sensor protrudes from the body of the user during analyte monitoring.

The electronics may be configured to receive the signal wirelessly from the sensor while the sensor is implanted within the body of the user.

The sensor may comprise a first portion coupled to the electronics and a second portion configured to be positioned in the body of the user for monitoring the level of the analyte of the user.

The sensor may comprise a proximal portion configured to be positioned above skin of the user and to be electrically coupled with electronics and a distal portion configured to be transcutaneously positioned through the user's skin and in contact with a bodily fluid of the user, wherein the distal portion of the sensor is further configured to detect an analyte in the bodily fluid and output a signal to the electronics.

The sensor may be configured to measure an analyte level.

The analyte may be glucose.

The analyte may be one or more ketones.

The analyte may be lactate.

The analyte may be oxygen.

The analyte may be ammonium.

The analyte may be asparagine.

The analyte may be aspartate.

The analyte may be cholesterol.

The analyte may be choline.

The analyte may be creatinine.

The analyte may be a diaphorase inhibitor.

The analyte may be ethanol.

The analyte may be glutamate.

The analyte may be glycerol.

The analyte may be lactose.

The analyte may be phosphate.

The analyte may be potassium.

The analyte may be pyruvate.

The analyte may be uric acid.

The analyte may be a first analyte, and the sensor may be further configured to measure a level of a second analyte of the user.

The first analyte may be glucose and the second analyte may be a ketone.

The first analyte may be glucose and the second analyte may be lactate.

The first analyte may be lactate and the second analyte may be a ketone.

The first and second analytes may be selected from the list consisting of: ammonium, asparagine, aspartate, cholesterol, choline, creatinine, diaphorase inhibitors, ethanol, glucose, glutamate, glycerol, one or more ketones, lactate, lactose, oxygen, phosphate, potassium, pyruvate, and uric acid.

The analyte sensor may be a glucose sensor comprising: a proximal portion configured to electrically couple with the electronics; a distal portion having a glucose-responsive enzyme, wherein the distal portion is configured to be positioned in a bodily fluid of the user's body and to sense glucose in the user's bodily fluid in a subcutaneous layer.

The device may further comprise a moisture permeable material attached to at least a portion of the base.

Providing such a material advantageously allows passage of water vapor therethrough, for removal of moisture from the region under the device, while blocking passage of liquid water, which could enter from externally. In effect, the moisture permeable material acts as an impediment to the free movement of liquid into features such as the vent and/or sensor passageway while allowing moisture vapor to exit. The moisture permeable material can impede movement of liquid through itself (and the vent and/or sensor passageway) freely and at scale, such as may be the case if the wearer were bathing or showering and subjecting the on-body device to immersion or a direct stream of liquid water.

The moisture permeable element may be arranged on the entirety of the first surface of the base.

The moisture permeable element may comprise a plurality of portions on the base.

The moisture permeable element may extend to a periphery of the base.

The moisture permeable element may be arranged within a recess in the base.

The moisture permeable material may be flush with the first surface of the base.

The base may comprise a plurality of surfaces configured to face towards the skin surface of the user, and the moisture permeable material may be located across the plurality of surfaces.

The base may comprise a plurality of surfaces configured to face towards the skin surface of the user, and the moisture permeable material may be located across at least a portion of one of the plurality of surfaces.

The device may further comprise a moisture permeable material within the at least one channel.

Combining channels with a moisture permeable material can enhance vapor transport while managing liquid ingress.

The moisture permeable material may extend along an entire length of the at least one channel.

The moisture permeable material may be located within the channel and across at least a portion of the first surface of the base.

The at least one channel may have a channel bottom located distal to a channel top, and a width at the channel bottom may be greater than a width at the channel top to secure the moisture permeable material within the channel.

As such, the at least one channel can have a dovetail shape to mechanically retain an inserted or moulded moisture permeable material.

The moisture permeable material may comprise silicone.

The moisture permeable material may comprise poly(methyl methacrylate).

The moisture permeable material may comprise a moisture permeable fibrous material.

The moisture permeable material may comprise polyurethane (PU).

The moisture permeable material may comprise polytetrafluoroethylene (PTFE).

The moisture permeable material may comprise polyethylene (PE).

The moisture permeable material may comprise nylon (polyamide).

The device may further comprise an adhesive patch attached to the housing and configured to attach the housing to skin of the user.

The adhesive patch may comprise an adhesive on a first side for coupling the adhesive patch to the base.

The adhesive patch may comprise an adhesive on a second side for adhering the adhesive patch to the skin of the user.

The adhesive patch may be attached to the housing and may be in contact with the moisture permeable material, and the moisture permeable material may be configured to allow water molecules to diffuse from the adhesive patch and through the moisture permeable material.

Advantageously, the moisture permeable material can effectively wick moisture away from the adhesive patch.

The adhesive patch may comprise one or more openings in one or more positions corresponding to the at least one channel.

The housing may be configured to be coupled to a body of the user.

The housing may encompass the electronics.

The housing may be a first housing, and the on-body device may comprise a second housing that encompasses the electronics.

As such, the housing may be a two-part housing configuration.

The second housing may be configured to couple with the first housing.

The first housing may be configured to hold the second housing and the electronics.

The first housing may be configured as a receptacle for the second housing.

The area adjacent the housing may comprise at least one of: space between the base and an adhesive; adhesive; a flexible patch; and a skin surface.

The on-body device may comprise an injection molded housing.

The on-body device may comprise a one-shot molded or over-molded housing.

The on-body device may comprise a housing comprising a first part and a second part assembled together with sensor electronics arranged between.

The on-body device may comprise a housing with a recess configured to receive a sensor connector, wherein the recess is arranged in a top of the housing or in a bottom of the housing.

The on-body device may comprise a housing with a recess configured to receive a sensor connector, wherein the recess is arranged in a top of the housing or in a bottom of the housing.

A periphery or a peripheral edge of the base of the housing may have a shape comprising one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; dodecagonal shape, or any other shape.

The shape of the peripheral edge of the base of the housing may comprise sharp and/or rounded corners.

The shape of the peripheral edge of base of the housing may comprise straight and/or curved edges.

At least some of the curved edges may have a constant radius of curvature.

At least some of the curved edges may have a variable radius of curvature.

The shape of the peripheral edge of the base of the housing may comprise one or more indentations and/or one or more protrusions or lobes.

The aperture may be located at a centroid of the shape of the peripheral edge of the base of the housing.

The aperture may be offset from a centroid of the shape of the peripheral edge of the base of the housing.

The offset may be along an axis of symmetry of the shape.

The offset may be along an axis that is perpendicular to an axis of symmetry of the shape and in the plane of the base.

The offset may be along both an axis of symmetry of the shape and an axis that is perpendicular to the axis of symmetry of the shape.

The aperture may be located in a corner of the shape and spaced apart from the periphery of the base.

The electronics may comprise a processor, memory, a power supply, and wireless communication circuitry configured to wirelessly communicate data indicative of a glucose level.

Accordingly, the device may be a one-piece device.

The electronics may be configured to be disposed after a single use.

An insertion system may comprise: the on-body device; and an applicator configured to position the on-body device on the skin of the user.

The applicator may be configured to position the sensor in the body of the user.

The applicator may be configured to advance the on-body device along a linear path toward the skin surface of the user, such that a sharp carrying the sensor may be configured to pierce the skin of the user at an angle substantially perpendicular to the skin of the user.

An analyte monitoring system may comprise: the on-body device; and a receiver unit configured to communicate with the on-body device for receiving data associated with the analyte level of the user.

The receiver unit may be configured to receive the data indicative of the monitored level of the analyte according to a Bluetooth or Bluetooth Low Energy (BLE) protocol.

The receiver unit may be configured to receive the data indicative of the monitored level of the analyte according to a Near Field Communication (NFC) protocol.

The analyte monitoring system may further comprise a plurality of receiver units, wherein the plurality of receiver units may comprise the receiver unit, wherein the plurality of receiver units may be configured to receive data indicative of the monitored level of the analyte from the on-body device, wherein the on-body device may be configured to authenticate the plurality of receiver units, and wherein the plurality of receiver units may comprise a smart phone and a smart watch.

A system may comprise: the insertion system; and the analyte monitoring system.

A method of monitoring an analyte of a user may comprise: applying an on-body device to a body of the user; and monitoring a level of the analyte with the on-body device, wherein the on-body device may be configured according to the preceding descriptions.

Monitoring the level of the analyte with the on-body device may comprise monitoring the level of the analyte with the on-body device for a wear duration without removing the on-body device from the body of the user.

The wear duration may be 7 days or greater.

The wear duration may be 14 days or greater.

The wear duration may be at least 21 days or greater.

The wear duration may be 30 days or greater.

The wear duration may be 45 days or greater.

The wear duration may be between 7 days and 60 days.

The wear duration may be 14 days, such as no more than 14 days.

The wear duration may be 15 days, such as no more than 15 days.

The wear duration may be 21 days, such as no more than 21 days.

The wear duration may be 28 days, such as no more than 28 days.

Improved moisture management through channels and/or texture can contribute to achieving these extended wear durations by enhancing adhesive reliability.

The method may further comprise transmitting, from the on-body device to a receiver unit, data indicative of the monitored level of the analyte.

The analyte may be a first analyte, and the method may further comprise monitoring a level of a second analyte with the on-body device.

The method may further comprise transmitting, from the on-body device to a receiver unit, data indicative of the monitored level of the first analyte and the monitored level of the second analyte.

The data indicative of the monitored level of the analyte may be transmitted according to a Bluetooth or Bluetooth Low Energy (BLE) protocol.

The data indicative of the monitored level of the analyte may be transmitted according to a Near Field Communication (NFC) protocol.

The receiver unit may be a smart phone.

The receiver unit may be a dedicated receiver unit.

The receiver unit may be a smart watch.

The method may further comprise transmitting, from the on-body device to a plurality of receiver units, data indicative of the monitored level of the analyte, wherein the plurality of receiver units may each be in an authenticated state at the on-body device, and wherein the plurality of receiver units may comprise a smart phone and a smart watch.

Applying the on-body device to the body of the user may comprise applying the on-body device in a position over a wholly in vivo sensor.

Applying the on-body device to the body comprises using an applicator to apply the on-body device to the skin of the user and/or to implant a sensor.

The method may further comprise wirelessly transmitting data indicative of the level of the analyte from the wholly in vivo sensor to the on-body device.

The data indicative of the monitored level of the analyte may be transmitted from the wholly in vivo sensor according to a Bluetooth or Bluetooth Low Energy (BLE) protocol.

The data indicative of the monitored level of the analyte may be transmitted from the wholly in vivo sensor according to a Near Field Communication (NFC) protocol.

A method of manufacturing an on-body device for monitoring an analyte level of a user may comprise: providing a housing; and providing electronics within the housing, wherein the electronics may be configured to receive a signal from a sensor, the signal indicative of a level of an analyte of a user, wherein the housing may be adapted to permit egress of moisture from an area adjacent the housing.

The method may further comprise providing an aperture in a first region in a base of the housing.

The method may further comprise providing at least one channel along a first surface of the base of the housing, wherein the at least one channel may be configured to permit egress of moisture from an area adjacent the first surface.

Providing the at least one channel may comprise: providing one or more first channels, wherein each of the one or more first channels may comprise a first end arranged at a periphery of the base, and a second end arranged at the first region of the base; and providing one or more second channels, wherein each of the one or more second channels may comprise a first end arranged at the periphery of the base and a second end arranged at the periphery of the base.

Providing the one or more second channels may comprise providing one or more curved channels that intersect the one or more first channels.

The method may further comprise applying a directional surface texture to a base of the housing.

The applying of the directional surface texture may be by a laser process, electrical discharge machining, or wire brush process.

The method may further comprise applying a non-directional surface texture to a base.

The applying of the non-directional surface texture may comprise a bead blasting process.

A method of manufacturing the on-body device may comprise: providing the housing; and providing the electronics within the housing, wherein the electronics may be configured to receive a signal from a sensor, the signal indicative of a level of an analyte of a user, wherein the housing may be adapted to permit egress of moisture from an area adjacent the housing.

A kit of parts may comprise: the on-body device; and an applicator configured to position the on-body device on the skin of the user.

During testing, the inventors figured out that if an adhesive patch is adhered to a user's skin and the edge of the patch begins to peel (i.e. to stop adhering properly to the skin), then the rest of the patch may cease to adhere surprisingly quickly after that. The inventors found this to be the case for several reasons. For instance, if the edge of the patch peels even slightly, the exposed adhesive may catch on nearby clothing, etc, which further acts to peel the patch away. Second, a properly adhered patch is generally difficult to remove as any forces pulling the patch off will be distributed evenly about the adhesive; however, this is not the case if an edge has started to peel as any forces applied to that edge will result in a stress concentration at the adhesive connecting the peeled edge to the skin.

As will be described in more detail below, the inventors have discovered that several approaches are effective at either minimising the likelihood of peel beginning or minimising the risk of the peel quickly progressing, or both.

The first approach is to provide an adhesive patch which comprises one or more removable patch portions releasably connected to the patch. These removable patch portions can be removed from, or detached from, the patch as desired by the user. The patch is configured such that the edges of the removable patch portions are likely to peel first. As will be explained further below, when the edge of a removable patch portion does peel, this removable patch portion can be removed thereby revealing or exposing a “fresh” or properly adhered edge; this may be described herein as “refreshing” the edge. Unlike the old peeling edge, the properly adhered edge should remain well adhered to the user's skin for a useful length of time. The removable patch portion can be releasably connected to the remaining patch in such a way that it is easy to remove the removable patch portion without causing the remaining patch to unstick from the skin. A plurality of removable patch portions may be provided so that the peeling areas of the patch edge can be precisely removed while minimising the amount of properly adhered edge that need be removed.

An aspect of the present disclosure provides an adhesive patch comprising a plurality of patch portions, each of the plurality of patch portions defining a lower surface configured to face a user's body in use and an upper surface configured to face away from the user's body in use, wherein the plurality of patch portions comprises: a primary patch portion having an outer periphery, and one or more removable patch portions, wherein each lower surface of the one or more removable patch portions comprises an adhesive layer for adhering to the user's body, wherein at least one of the one or more removable patch portions is releasably coupled to the primary patch portion.

By providing a removable patch portion, it is possible to remove the removable patch portion if one or more of its edges start to peel. In this way, a non-peeling edge of another part or portion of the patch may be revealed, thus “refreshing” the edge and thereby extending the wear duration.

The lower surface of the primary patch portion may comprise an adhesive layer.

The adhesive layer of the primary patch portion may comprise a pressure sensitive adhesive, which may be an acrylic based pressure sensitive adhesive.

The thickness of the adhesive layer of the lower surface of the primary patch portion may be between 50 μm to 125 μm or 75 μm to 100 μm.

The adhesive patch may comprise one or more release liners covering the adhesive layer of the primary patch portion.

The adhesive patch may comprise one or more release liners covering the respective adhesive layers of the one or more removable patch portions.

The adhesive patch may comprise one or more release liners covering each of the adhesive layers of the plurality of patch portions.

The one or more release liners may be contiguous and/or may form a continuous surface.

The release liner may comprise silicone coated paper or silicone coated polyester film.

At least one, or all, of the respective adhesive layers of the one or more removable patch portions may comprise a pressure sensitive adhesive, which may be an acrylic based pressure sensitive adhesive.

The thickness of at least one, or each, of the adhesive layers of the removable patch portions may be between 50 μm to 125 μm or 75 μm to 100 μm

The upper surface of the primary patch portion may comprise a device adhesive for securing an on-body device to the adhesive patch, wherein the device adhesive may comprise a pressure sensitive adhesive, which may be an acrylic based pressure sensitive adhesive.

The primary patch portion and/or the one or more removable patch portions may comprise one or more backing layers defining the respective upper surface and the lower surface.

At least one, or each, of the one or more backing layers may have a thickness between 10 μm and 300 μm, between 15 μm and 275 μm, between 40 μm and 200 μm, or between 50 μm and 150 μm.

The primary patch portion may comprise an aperture through which a cannula or sensor of an on-body device may pass.

Providing an aperture may allow the cannula or sensor of the on-body device to pass into the body more easily.

The primary patch portion may comprise an aperture sized to at least partially pass over the top of an on-body device when the on-body device is adhered to the user's body.

The primary patch portion may comprise a substantially rigid locating feature configured to mate with the on-body device.

Use of an aperture sized to pass over the on-body device or a rigid locating feature may allow the adhesive patch to better secure an existing on-body device.

The substantially rigid locating feature may form an aperture sized to at least partially pass over the top of an on-body device when the on-body device is adhered to the user's body.

The on-body device may comprise an injection molded housing.

The on-body device may comprise a one-shot molded or over-molded housing.

The on-body device may comprise a housing comprising a first part and a second part assembled together with sensor electronics arranged between.

The on-body device may comprise a housing with a recess configured to receive a sensor connector, wherein the recess is arranged in a top of the housing or in a bottom of the housing.

The outer periphery of the primary patch portion may be substantially circle or oval shaped.

The outer periphery of the primary patch portion may be substantially polygon shaped. The polygon shape may, for example, be a rectangle shape or trapezoid shape.

At least one, or all, of the corners of the polygon shape may be rounded.

The outer periphery may define a shape comprising one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. Other shapes can also be used.

At least one, or all, of the plurality of patch portions may be formed from a non-woven material, and the non-woven material may be a polyurethane or polyester spunlace material.

The one or more removable patch portions may be a plurality of removable patch portions, wherein each of the plurality of removable patch portions may be releasably coupled to another one of the patch portions.

Use of a plurality of removable patch portions may allow for an edge of the patch to be “refreshed” multiple times or for multiple different edges of the patch to be “refreshed”.

The plurality of removable patch portions may comprise one or more first removable patch portions, wherein each of the one or more first removable patch portions may be releasably coupled to the primary patch portion

At least one of the one or more first removable patch portions may be releasably coupled to the primary patch portion and to another of the one or more first removable patch portions.

At least one of the one or more first removable patch portions may be releasably coupled to the primary patch portion and to another two of the one or more first removable patch portions.

The one or more first removable patch portions may comprise or consist of two removable patch portions.

The one or more first removable patch portions may comprise or consist of three removable patch portions.

The one or more first removable patch portions may comprise or consist of four removable patch portions.

The one or more first removable patch portions may comprise or consist of five removable patch portions.

The one or more first removable patch portions may comprise or consist of between six and ten removable patch portions.

At least one, or each, of the one or more first removable patch portions may have a polygonal shape. The polygonal shape may be a rectangular or trapezoidal shape.

At least one, or each, of the one or more first removable patch portions may have a crescent shape.

At least one, or each, of the one or more first removable patch portions may have a one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Each of the one or more first removable patch portions may be the same shape and/or may be the same size.

The plurality of removable patch portions may comprise one or more second removable patch portions, wherein each of the one or more second removable patch portions may be not releasably coupled to the primary patch portion but may be releasably coupled to at least one of the one or more first removable patch portions.

Use of second removable patch portions may provide an additional opportunity to “refresh” an edge in a given direction relative to using only a first removable patch portion.

At least one of the one or more second removable patch portions may be releasably coupled to at least another of the one or more second removable patch portions.

At least one of the one or more second removable patch portions may be releasably coupled to at least two of the one or more second removable patch portions.

Each of the one or more second removable patch portions may be releasably coupled to at least two of the one or more second removable patch portions.

The one or more second removable patch portions may comprise or consists of two removable patch portions.

The one or more second removable patch portions may comprise or consists of three removable patch portions.

The one or more second removable patch portions may comprise or consists of four removable patch portions.

The one or more second removable patch portions may comprise or consists of five removable patch portions.

The second one or more removable patch portions may comprise or consist of between six and ten removable patch portions.

At least one, or each, of the one or more second removable patch portions may have a polygonal shape. The polygonal shape may, for example, be a rectangular or trapezoidal shape.

At least one, or each, of the one or more second removable patch portions may have a crescent shape.

At least one, or each of the one or more second removable patch portions may have a one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Each of the one or more second removable patch portions may be the same shape and/or may be the same size.

At least one, or all, of the one or more first removable patch portions may be smaller than at least one, or all, of the one or more second removable patch portions.

At least one, or all, of the one or more first removable patch portions may be larger than at least one, or all, of the one or more second removable patch portions.

The plurality of removable patch portions may comprise one or more third removable patch portions, wherein each of the one or more third removable patch portions may be not releasably coupled to the primary patch portion or to the one or more first removable patch portions.

Use of third removable patch portions may provide an additional opportunity to “refresh” an edge in a given direction relative to using only first and second removable patch portion.

Each of the one or more third removable patch portions may be indirectly coupled to the primary patch portion.

At least one of the one or more third removable patch portions may be releasably coupled to at least one of the one or more second removable patch portions.

At least one, or each, of the one or more third removable patch portions may have a polygonal shape. The polygonal shape may, for example, be a rectangular or trapezoidal shape.

At least one, or each, of the one or more third removable patch portions may have a crescent shape.

At least one, or each, of the one or more third removable patch portions may have one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Each of the one or more third removable patch portions may have the same shape, may have the same size, and/or may have the same size and shape.

At least one, or all, of the one or more first removable patch portions may be smaller than at least one, or all, of the one or more third removable patch portions.

At least one, or all, of the one or more first removable patch portions may be larger than at least one, or all, of the one or more third removable patch portions.

At least one, or all, of the one or more second removable patch portions may be smaller than at least one, or all, of the one or more third removable patch portions.

At least one, or all, of the one or more second removable patch portions may be larger than at least one, or all, of the one or more third removable patch portions.

The plurality of patch portions may extend in or along substantially the same plane or curved surface.

The primary patch portion may be entirely encompassed by the plurality of removable patch portions.

Each of the one or more first removable patch portions may be releasably coupled to, or proximate to, the outer periphery of the primary patch portion.

The primary patch portion and the one or more first removable patch portions may extend in or along substantially the same plane or curved surface.

The primary patch portion may be entirely encompassed by the one or more first removable patch portions.

At least 30%, at least 90%, or about 100%, of the length of the outer periphery of the primary patch portion may be releasably connected to the one or more first removable patch portions.

The one or more first removable patch portions may be uniformly distributed about the outer periphery of the primary patch portion.

The one or more first removable patch portions may be located with a rotational symmetry of order two or more with respect to a point within the primary patch portion.

The order of the rotational symmetry may be equal to the number of first one or more removable patch portions.

The primary patch portion may be entirely encompassed by the one or more second of the removable patch portions.

The one or more second of the removable patch portions may be uniformly distributed about outer periphery of the primary patch portion.

The one or more second removable patch portions may be located with a rotational symmetry of order two or more with respect to a point within the primary patch portion.

The order of the rotational symmetry may be equal to the number of the one or more second removable patch portions.

The primary patch portion may be entirely encompassed by the one or more third removable patch portions.

The primary patch portion may be entirely encompassed by a combination of the one or more first removable patch portions and the one or more second removable patch portions.

The primary patch portion may be entirely encompassed by a combination of the one or more first removable patch portions and the one or more third removable patch portions.

The primary patch portion may be entirely encompassed by a combination of the one or more first removable patch portions, the one or more second removable patch portions and the one or more third removable patch portions.

Where the primary patch portion is entirely encompassed, it may be possible to “refresh” a peeling edge in any given outward direction. Where multiple sets of removable patch portions each fully encompass the primary patch portion, it may be possible to “refresh” peeling edges multiple times in any given direction.

The primary patch portion may not be entirely encompassed by the one or more first removable patch portions.

The primary patch portion may not be entirely encompassed by the one or more second removable patch portions.

The primary patch portion may not be entirely encompassed by the one or more third removable patch portions.

The plurality of removable patch portions may form one or more concentric rings around the primary patch portion.

The plurality of removable patch portions may be releasably connected to adjacent ones of the primary patch portion and plurality of removable patch portions.

The one or more first removable patch portions may be releasably connected to the lower surface of the primary patch portion.

By providing removable patch portions releasably coupled to the lower surface of the primary patch portion, it may be possible to provide a more compact adhesive patch (e.g. that extends less outwardly) since the removable patch portions need not extend outwardly relative to the primary patch portion.

The lower surface of the primary patch portion may comprise an adhesive layer, and the one or more first removable patch portions may be releasably connected to the adhesive layer of the primary patch portion.

The upper surfaces of the one or more first removable patch portions may comprise a release liner configured to adhere to the adhesive layer of the primary patch portion less strongly than the user's body would adhere to the adhesive layer.

The primary patch portion may extend in or along a first plane or curved surface and the one or more first removable patch portions extend in or along a second plane which may be offset from but substantially parallel to the first plane.

The lower surface of the primary patch portion may be at least 30% covered, at least 90% covered, or about 100% covered, by the one or more first removable patch portions.

The one or more first of removable patch portions may form a grid pattern.

At least one of the one or more first removable patch portions may not be coupled to any other one of the one or more first removable patch portions or to any other one of the plurality of removable patch portions.

The one or more first removable patch portions may define a continuous or discontinuous outer periphery.

At least part, or all of, of the outer periphery of the one or more first removable patch portions may be more outward than at least part of, or all of, the outer periphery of the primary patch portion.

At least part of, or all of, of the outer periphery of the one or more first removable patch portions may be coincident with the outer periphery of the primary patch portion.

Each of the one or more first removable patch portions may extend to the outer periphery of the one or more first removable patch portions.

The outer periphery of the one or more first removable patch portions may encompass a greater area than the outer periphery of the primary patch portion.

The outer periphery of the one or more first removable patch portions may encompass a smaller area than the outer periphery of the primary patch portion.

At least one of the one or more removable patch portions may define a path-following removable patch portion, wherein the path-following removable patch portion may be configured to follow a path as it is progressively removed from the adhesive patch.

Use of a path-following removable patch portion may allow for particularly intuitive or easy removal of patch portions.

The adhesive patch and/or the path-following removable patch portion may comprise an indica or marker indicating a predetermined point from which the path-following removable patch portion should be removed so that it follows the path.

Use of an indicia may clearly indicate how the path-following removable patch portion is to be used.

The path may be concertina shaped.

The path may double back on itself.

The path may be spiral shaped. The spiral may be a square or rectangular spiral or may be a curved spiral.

The path-following removable portion may comprise one or more releasable self-couplings that removably couple the path-following removable patch portion to itself, wherein the one or more releasable self-couplings may be configured such that the path-following removable patch portion preferentially follows the path as it may be removed from the patch.

The path-following removable patch portion may comprise one or more releasable self-couplings that removably couple the path-following removable patch portion to itself, wherein the one or more releasable self-couplings may be configured to break as the path-following removable patch portion may be removed along the path.

The one or more releasable self-couplings may comprise one or more partial releasable self-couplings that extend only partially into the path-following removable portion, such that the one or more partial releasable self-coupling break as the path-following removable patch portion is removed.

Use of releasable self-couplings may ensure that the path-following removable patch portion follows its path in a predictable manner.

At least one of the releasable self-couplings may extend from a respective edge of the path-following removable portion to a respective point within the path-following removable patch portion.

At least one of the one or more partial releasable self-couplings may be substantially straight.

At least one of the one or more partial releasable self-couplings may be substantially curved.

At least one of the one or more partial releasable self-couplings may follow a spiral shape.

The one or more releasable self-couplings may comprise at least two partial releasable self-coupling that may be substantially parallel and offset relative to each other.

The one or more releasable self-couplings may comprise at least two partial releasable self-coupling, which extend from opposite edges of the path-following removable patch portion.

The at least two partial releasable self-coupling may overlap without contact.

The path-following removable patch portion may be bounded by an outer periphery and an inner periphery, and wherein the one or more releasable self-couplings may comprise a guiding releasable self-coupling that extends from the outer periphery to the inner periphery of the path-following removable patch portion.

The guiding releasable self-coupling may be substantially straight.

The guiding releasable self-coupling may be substantially curved.

The guiding releasable self-coupling may follow a spiral.

The guiding releasable self-coupling may double back on itself and/or may be concertinaed.

The inner periphery of the path-following removable portion may be releasably coupled to the outer periphery of the primary patch portion.

The inner periphery of the path-following removable portion may be releasably coupled to the outer periphery of the primary patch portion by a releasable coupling extending along the outer periphery of the primary patch portion.

The path-following removable portion may be releasably connected to the outer periphery of the primary patch portion.

The path-following removable portion may be releasably connected to the lower surface of the primary patch portion.

The path-following removable patch portion may be defined by at least two removable patch portions, wherein the at least two removable patch portions may be arranged as a lengthways sequence along a length of the path.

The path-following removable patch portion may be defined by at least two removable patch portions, wherein the at least two removable patch portions may be delineated by at least one releasable coupling extending completely or partially across the path.

The at least one releasable coupling extending completely or partially across the path may extend substantially orthogonal to direction of the path at that location.

The path-following removable patch portion may consist of removable patch portions of the one or more first removable patch portions, the one or more second removable patch portions, and/or the one or more third removable patch portions.

At least one, or each, releasably coupled pair of portions may be releasably coupled by a releasable coupling.

The releasable coupling may comprise or consist of at least one line of weakness or frangible boundary.

The releasable coupling may delineate an otherwise continuous portion into the two releasably coupled portions.

The releasable coupling may be at least partly formed by at least part of the adhesive layer of one of the pair of releasably coupled patch portions contacting the upper surface of the other of the pair of releasably coupled patch portions.

The releasable coupling may be at least partly, or entirely, formed by perforations.

The releasable coupling may be at least partly, or entirely, formed by a thinning of the material forming the pair of releasably coupled portions.

The thinning may be through an upper surface of the material, such that the upper surface along at least part of the releasable coupling is relatively lower than the upper surface of the material immediately away from the releasable coupling.

The thinning may be through a lower surface of the material, such that the lower surface along at least part of the releasable coupling is relatively higher than the lower surface of the material immediately away from the releasable coupling.

The thinning may result in at least part or all of the releasable coupling having a thickness that is less than on either immediate side of the releasable coupling.

The releasable coupling may be at least partly, or entirely, formed by an adhesive coupling two or more otherwise non-contacting edges of the pair of releasably coupled portions.

The shared lower surface of a pair of releasably coupled portions may comprise no adhesive layer along at least part, or all, of the releasable coupling.

The shared lower surface of a pair of releasably coupled portions may comprise a relatively weaker adhesive layer along at least part, or all, of the releasable coupling when compared to the adhesive layer.

A shared lower surface of a pair of releasably coupled portions may comprise no adhesive layer or a weaker adhesive along at least part, or all, of the releasable coupling when compared to the adhesive layer.

The outer periphery of the primary patch portion and/or an outer periphery of at least one of the one or more removable patch portions may define at least one outside corner.

At least one, or all, of the outside corners may have an external angle less than 315 degrees or less than 225 degrees.

At least one, or all, of the outside corners may be blunt.

At least one, or all, of the outside corners may be radiused with a radius of at least 0.3 mm or a radius of at least 3 mm.

Any outside corner having an external angle greater than 315 degrees may have a radius of at least 0.5 mm, and/or any outside corner having an external angle less than 225 degrees may have no radius.

By having external corners having maximum angle lower than a desired maximum or if the external corners are radiused with a radius of at least a desired minimum, the corner of any given removable patch portion may be adhered with an appropriate amount of adhesive.

At least one of the one or more removable portions may tessellate with an adjacent one of plurality of patch portions.

Each one of the one or more removable portion may tessellate with an adjacent one of the primary patch portion and/or another of the one or more removable patch portions.

Each one of the one or more removable patch portions may tessellate with all adjacent ones of the plurality of patch portions.

Before any of the one or more removable patch portions have been removed, it may be that no gaps exist between any adjacent members of the plurality of patch portions.

At least one, or all, of the one or more removable patch portions may comprise a pull tab. One or all of the pull tabs may comprise no adhesive layer on its lower surface.

The pull tab may extend from an edge of the removable patch portion and/or may extend from the upper surface of the removable patch portion.

The adhesive patch may be configured so that if an edge forming part of the outer patch periphery starts to peel from or become improperly adhered to the user's body, one or more removable patch portions defining that edge can be removed to expose an edge that may be not starting to peel from or may be properly adhered to the user's body.

At least one of the one or more removable patch portions may comprise a partial releasable coupling extending from an edge of the removable portion to a point within the removable portion, wherein the partial releasable coupling may assist in removal of the removable portion.

The partial releasable coupling may extend substantially orthogonally from the edge of the removable portion.

The partial releasable coupling may extend substantially non-orthogonally from the edge of the removable portion.

The primary patch portion and the one or more removable patch portions may define an outer patch periphery, wherein the adhesive patch may comprise at least one releasable coupling extending from a location on the outer patch periphery to assist in removal of the one or more removable patch portions.

The at least one releasable coupling may extend substantially orthogonally to the outer patch periphery at the location.

Distal from the outer patch periphery, the at least one releasable coupling may extend non-orthogonally to or parallel with the edge of the outer patch periphery at the location.

The at least one releasable coupling may extend substantially non-orthogonally from the outer patch periphery at the location.

The at least one releasable coupling may be configured such that at least one removable portion adjacent to the releasable coupling may be configured to be removed in a circumferential direction and/or parallel with the outer patch periphery proximal to that location.

The partial releasable coupling and/or at least one releasable coupling may be provided or formed as discussed herein.

Another aspect provides an on-body device patch comprising an on-body device and the adhesive patch.

The on-body device may be secured to a device adhesive on the upper surface of the primary patch portion of the primary patch portion.

The on-body device may be an analyte monitor.

The on-body device may be a glucose monitor or a continuous glucose monitor.

The adhesive patch may comprise an anti-stick layer.

The anti-stick layer may be located at or on the upper surface of the primary patch portion.

The anti-stick layer may be located at or on the upper surface of the one or more removable patch portions.

The anti-stick layer may be located at or on the upper surface of the on-body device.

The anti-stick layer may comprise a fluoropolymer or silicone.

The anti-stick layer may be a permanent coating.

The anti-stick layer may be formed in or inherent to the material of the primary patch portion, one or more removable patch portions, or on-body device housing.

The anti-stick layer may self-adhere to the material of the primary patch portion, one or more removable patch portions, or on-body device housing.

The anti-stick layer may be a removable sticker.

The removable sticker may comprise a weak adhesive and/or wherein the adhesive of the removable sticker may be weaker than an, or all, adhesive layers of the adhesive patch.

The inventors have found that the use of an overpatch can prolong wear duration by increasing the surface area of contact of the on-body device with the wearer's skin, by providing a sticky surface that is replaceable one or more times during the wear of the on-body device, by providing a mechanism to hold down a peripheral edge of the main adhesive patch of the on-body device, and/or by providing an adhesive patch that is more resilient to movement of the skin and more durable. Aspects of overpatch design that provide for increased wear duration are further described herein. Some of these aspects are similarly applicable to the main adhesive patch of the on-body device.

Use of an anti-stick component may minimise the likelihood of an overpatch sticking to an on-body device patch when applied. Additionally, the overpatch may be more easily positioned or may need to be positioned less precisely when using an anti-stick component.

A method of applying the adhesive patch, the method comprising applying and adhering the adhesive patch to a user's skin.

The adhesive patch may be the on-body device patch.

The method may further comprise using an applicator tool to apply the on-body device patch to the user's skin.

The method may further comprise applying a further one of the adhesive patch over the on-body device patch, wherein the further adhesive patch may be an overpatch.

The adhesive patch may be applied over a pre-existing on-body device patch, wherein the adhesive patch may be an overpatch.

The method may further comprise placing a sticker having an anti-stick upper surface onto an upper surface of the on-body device patch before applying the overpatch.

Applying the overpatch may further comprise adhering the further adhesive patch to the skin or body surrounding the on-body device patch.

Applying the overpatch may comprise aligning an aperture or mating feature with the on-body device of the on-body device patch.

A method of monitoring an analyte using the on-body device patch or using an on-body device secured using the adhesive patch, the method comprising monitoring the analyte.

The analyte may be glucose.

The analyte may be monitored substantially continuously.

The on-body device patch and/or the adhesive patch may be applied as discussed herein.

In another approach, a reinforcing element (e.g. a leg, ring or band) may extend from, e.g., the on-body device to contact the upper surface of the edge of the patch. The reinforcing element may simply rest on the upper surface or it may apply a force to the upper surface. In either case, the reinforcing element acts to hold down the edge and prevent it from peeling. In some aspects, this may result in the centre of the patch being raised slightly (as a reaction force); however, the centre of the patch is less likely to peel as it is not exposed, thus this slight reaction force is not problematic.

In one aspect a reinforcement device is provided for reinforcing attachment of an adhesive patch to a skin surface of a user. The adhesive patch is coupled to a housing of an on-body device. The reinforcement device comprises one or more reinforcement legs. Each of the one or more reinforcement legs comprises a first end configured to be directly or indirectly coupled to the housing and a second end configured to contact the adhesive patch.

The one or more reinforcement legs may comprise a plurality of reinforcement legs.

The plurality of reinforcement legs may be configured to be arranged around the perimeter of the housing when coupled directly or indirectly to the housing. The plurality of reinforcement legs may be spaced evenly around the perimeter of the housing. This ensures that the adhesive patch is reinforced evenly.

Each of the plurality of reinforcement legs may define a respective longitudinal axis and a projection of the respective longitudinal axis of each reinforcement leg in a plane of the skin surface may be approximately aligned with a radial axis extending from a center of the housing.

Each of the plurality of reinforcement legs may extend downwardly from the housing towards the skin surface. Alternatively, each of the plurality of reinforcement legs extend parallel to the adhesive patch.

The second end of each reinforcement leg may comprise a foot. Each foot may be configured to be adhered to the adhesive patch. A base surface of each foot may comprise an adhesive layer.

Each foot may be configured to extend radially beyond a periphery of the adhesive patch. A portion of each foot may be configured to overlay the adhesive patch and a portion of each foot may be configured to overlay the skin surface.

An outer edge of each foot may be configured to extend radially to and not beyond a periphery of the adhesive patch.

The first end of each reinforcement leg may be configured to be directly coupled to the housing.

The first end of each reinforcement leg may be configured to be coupled to the housing by a respective hinge. Each hinge may be a living hinge.

The first end of each reinforcement leg may be configured to be coupled to the housing by a respective snap-fit joint.

The plurality of reinforcement legs may be formed integrally with the housing.

The first end of each reinforcement leg may be directly coupled to a body portion of the reinforcement device, wherein the body portion is configured to be coupled to the housing.

The first end of each reinforcement leg may be configured to be coupled to the body portion by a respective hinge. Each hinge may be a living hinge

The first end of each reinforcement leg may be configured to be coupled to the body portion by a respective snap-fit leg joint.

The plurality of reinforcement legs may be formed integrally with the body portion.

The body portion may be configured to be coupled to the housing by one or more snap-fit body joints. The one or more snap-fit body joints may comprise a snap-fit body joint that extends around the perimeter of the housing and/or the body portion

The body portion may comprise an inner edge defining an opening for receiving the housing and an outer edge.

The body portion may be formed of an elastomeric material and the inner edge may correspond to a shape and size of the housing such that the inner edge of the body portion is configured to grip the housing.

The inner edge may have a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

The shape of the inner edge may comprise sharp and/or rounded corners.

The shape of the inner edge may comprise straight and/or curved edges. At least some of the curved edges may have a constant radius of curvature. At least some of the curved edges may have a variable radius of curvature.

The shape of the inner edge may comprise one or more indentations and/or one or more protrusions or lobes.

At least part of the body portion may be configured to overlay at least part of the upper surface of the housing. The entirety of the body portion may be configured to overlay at least part of the upper surface of the housing. The body portion may be configured to overlay the entirety of the upper surface of the housing.

An underside of the body portion may be configured to be adhered to the upper surface of the housing.

The body portion may comprise a plate, wherein an outer edge of the plate defines an outer edge of the body portion.

The outer edge of the body portion may have a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

The shape of the outer edge may comprise sharp and/or rounded corners.

The shape of the outer edge may comprise straight and/or curved edges. At least some of the curved edges may have a constant radius of curvature. At least some of the curved edges may have a variable radius of curvature.

The shape of the outer edge may comprise one or more indentations and/or one or more protrusions or lobes.

The body portion may comprise a plurality of apertures extending entirely through the body portion.

The body portion may be formed form a moisture permeable material.

The body portion may be formed from a rigid material.

The reinforcement legs may be configured to be at least partially covered by the body portion in use.

The reinforcement legs may be configured to be fully covered by the body portion when in use.

The plurality of feet may be coupled by a connection member. The connection member may be configured to adhere to the adhesive patch. An underside of the connection member may comprise an adhesive layer.

The connection member may comprise an inner edge and an outer edge.

The inner edge of the connection member and/or the outer edge of connection member may have a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

The shape of the inner edge of the connection member and/or the outer edge of connection member may comprise sharp and/or rounded corners.

The shape of the inner edge of the connection member and/or the outer edge of connection member may comprise straight and/or curved edges. At least some of the curved edges may have a constant radius of curvature. At least some of the curved edges may have a variable radius of curvature.

The shape of the inner edge of the connection member and/or the outer edge of connection member may comprise one or more indentations and/or one or more protrusions or lobes.

The body portion may comprise the connection member. The plurality of feet may be formed integrally with the connection member.

The reinforcement legs may be formed from a rigid material.

The reinforcement legs and body portion may be formed from different materials. The material from which the reinforcement legs are formed from may be more flexible than the material from which the body portion is formed. The material from which the reinforcement legs are formed may be more flexible than the material from which the housing is formed.

Each reinforcement leg may be biased such that the second end exerts a force on the adhesive patch in a direction towards the skin surface. The biasing may be provided by a spring member. The reinforcement legs may be provided with a length such that they are configured to bend when coupled directly or indirectly to the housing.

A cross section of each reinforcement leg may be arcuate. When each reinforcement leg is coupled directly or indirectly to the housing, the each reinforcement leg may be convex with respect to the skin surface. The arcuate shape of each leg may be configured to allow bending of the second end of each leg towards the skin surface but resist against bending of the second end of each leg away from the skin surface.

The reinforcement device may comprise at least 4, at least 8, or at least 12 reinforcement legs.

According to an aspect there is provided an analyte monitoring system, comprising a reinforcement device as described and an on-body device. The on-body device may comprise the housing; an analyte sensor comprising a first portion arranged within the housing and a second portion configured to be positioned below a skin surface and in contact with a bodily fluid to generate a signal indicative of an analyte level of a user; and sensor electronics arranged within the housing and coupled to the first portion of the analyte sensor, wherein the sensor electronics comprise a processor and wireless communications circuitry. The adhesive patch may be coupled to a first side of the housing and configured to secure the first side of the housing to the skin surface of the user.

The analyte may be one or more of: glucose, ammonium, asparagine, aspartate, cholesterol, choline, creatinine, diaphorase inhibitors, ethanol, glutamate, glycerol, one or more ketones, lactate, lactose, oxygen, phosphate, potassium, pyruvate, and uric acid.

The on-body device may be a glucose monitor.

The body portion may comprise an anti-stick layer. The anti-stick layer may be located at or on an upper surface of the body portion.

The on-body device may comprise an anti-stick layer. The anti-stick layer may be located at or on an upper surface of the on-body device.

The anti-stick layer may comprise a fluoropolymer or silicone.

The anti-stick layer may be a permanent coating.

The anti-stick layer may be formed in or inherent to the material of the body portion or the on-body device.

The anti-stick layer may self-adhere to the material of the primary patch portion, one or more removable patch portions, or on-body device housing.

The anti-stick layer may be a removable sticker. The removable sticker may comprise a weak adhesive.

According to an aspect there is provided a reinforcement device for reinforcing the attachment of an adhesive patch to a skin surface of a user. The adhesive patch is coupled to a housing of an on-body device. The reinforcement device comprises one or more reinforcement bands. Each of the one or more reinforcement bands comprises an inner edge defining an opening for receiving the housing and a lower surface configured to overlay the adhesive patch.

Each of the one or more reinforcement bands may comprise an outer edge. The outer edge may be configured to extend radially to a peripheral edge of the adhesive patch such that each of the one or more reinforcement bands reinforces the peripheral edge of the adhesive patch.

The outer edge may be configured to extend radially beyond a peripheral edge of the adhesive patch such that each of the one or more reinforcement bands reinforces the peripheral edge of the adhesive patch. Using an outer edge that extends beyond the peripheral edge of the adhesive patch means that the lower surface of the reinforcement band effectively secures the peripheral edge against the skin surface to prevent peeling.

The lower surface may comprise an adhesive layer configured to adhere the lower surface to the skin surface.

The inner edge may be configured to exert a force on the housing and thereby grip the housing when in use. This may be the means by which the reinforcement band is coupled to the housing.

At least one of the one or more reinforcement bands may be configured to mechanically couple to the housing.

At least one of the one or more reinforcement bands may be formed from an elastomeric material.

At least one of the one or more reinforcement bands may be formed from a shape-memory material.

At least one of the reinforcement bands may be rigid. At least one of the one or more reinforcement bands may be formed from a rigid polymer.

The opening may be configured such that an entire upper surface of the housing can extend through the opening.

The one or more reinforcement bands may comprise a plurality of reinforcement bands.

The plurality of reinforcement bands may be stacked on top of one another such that the lower surface of a second reinforcement band of the plurality of reinforcement bands contacts an upper surface of a first reinforcement band of the plurality of reinforcement bands.

Each of the plurality of reinforcement bands may be stacked parallel to an axis of the housing.

Each of the plurality of reinforcement bands may be arranged in a concentric manner such that the inner edge of a second reinforcement band of the plurality of reinforcement bands contacts or overlaps with an outer edge of a first reinforcement band of the plurality of reinforcement bands.

Each of the plurality of reinforcement bands may be frangibly connected to a neighboring one of the plurality of reinforcement bands.

The inner edge of the second reinforcement band may be frangibly connected to the outer edge of the first reinforcement band such that, in use, the second reinforcement band is removable from the skin surface of the user while the first reinforcement band remains in place. The reinforcement device may further comprise a third reinforcement band, wherein the inner edge of the third reinforcement band is frangibly connected to the outer edge of the first reinforcement band such that, in use, the third reinforcement band is removable from the skin surface of the user while the second reinforcement band remains in place.

The lower surface of the second reinforcement band may be frangibly connected to the upper surface of the first reinforcement band such that, in use, the second reinforcement band is removable from the skin surface of the user while the first reinforcement band remains in place. The reinforcement device may further comprise a third reinforcement band, wherein the lower surface of the third reinforcement band is frangibly connected to the upper surface of the second reinforcement band such that, in use, the third reinforcement band is removable from the skin surface of the user while the second reinforcement band remains in place.

A diameter of the outer edge of the second reinforcement band may be greater than a diameter of the outer edge of the first reinforcement band. A diameter of the outer edge of the third reinforcement band is greater than a diameter of the outer edge of the second reinforcement band.

Each of the reinforcement bands may be arranged concentrically and may not overlap with each other.

A ratio of a height of each reinforcement band to a width of the each reinforcement band may be greater than 1, wherein the height of each reinforcement band is defined between the lower surface of each reinforcement band and an upper surface of each reinforcement band, and, wherein the width of each reinforcement band is defined between the inner and outer edges of each reinforcement band. The ratio of the height of each reinforcement band to the width of the each reinforcement band may be greater than 2. The ratio of the height of each reinforcement band to the width of the each reinforcement band may be greater than 3. Increasing the ratio ensures that, upon concave flexing of the adhesive patch, adjacent reinforcement bands abut against each other, preventing further flexing and peeling of the adhesive patch. Furthermore, this arrangement permits convex flexing of the adhesive patch thus allowing the patch to stay adhered as the skin surface moves.

Each of the plurality of bands may be connected by one or more webbing members. The webbing members may extend radially between the concentric bands.

The plurality of concentric bands may be covered by a guard band.

At least two of the plurality of reinforcement bands may be formed of at least two different materials. The materials may be selected to provide differing rigidity, adhesion, and/or moisture wicking properties.

The rigidity of the plurality of reinforcement bands may decrease in a radially outward direction. This helps to ensure that the outermost bands can conform well with the skin surface.

The adhesion of the plurality of reinforcement bands may decrease in a radially outward direction. This may reduce the likelihood that an inner band is inadvertently removed when an outer band is removed from the skin surface. Furthermore, the inner bands are configured to remain attached to the skin surface for a longer duration and therefore may require stronger adhesion. The adhesion of the plurality of reinforcement bands may increase in a radially outward direction. This may prevent peeling of the outermost edge.

The moisture wicking ability of the material may decrease in a direction away from the skin surface. Moisture build up may be more likely to be problematic under the inner bands which are designed to be applied to the skin for a longer time period.

A first reinforcement band of the one or more reinforcement bands may comprise a plurality of radially extending channels each defining a gap between the adhesive patch and the first reinforcement band. Each of the plurality of radially extending channels may extend from the inner edge of the first reinforcement band to the outer edge of the first reinforcement band such that each gap extends from the inner edge of the first reinforcement band to the outer edge of the first reinforcement band. Each of the plurality of channels may be configured to allow convex curving of the first reinforcement band and resist concave curving of the first reinforcement band. A cross section of each channel along a plane perpendicular to a longitudinal axis of the each channel may be arcuate.

The one or more reinforcement bands may comprise a plurality of discrete beads, wherein the plurality of discrete beads are arranged in a plurality of radially extending rows of beads, wherein each row of beads is configured such that, when an adjacent area of the adhesive patch begins to peel away from the skin surface, each bead abuts against its neighbour, thus preventing further peeling of the adhesive patch. Each row of beads may comprise at least three beads. A face of each bead that is configured to abut against a neighbouring bead may be flat. Each bead may have the shape of a prism and may be arranged such that a longitudinal axis of each prism is oriented parallel to the row of beads of which the each bead forms part. Optionally, each bead may have the shape of a cuboid.

At least one of the one or more reinforcement bands comprises a plurality of apertures extending entirely through the reinforcement band. Each aperture of the plurality of apertures may have an oval shape. A minor axis of each aperture of the plurality of apertures may be approximately aligned with a radial axis extending from a center of the opening.

The inner edge of each of the one or more reinforcement bands may have a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The inner edge of each of the one or more reinforcement bands may comprise sharp and/or rounded corners. The shape of the inner edge of each of the one or more reinforcement bands comprises straight and/or curved edges. At least some of the curved edges may have a constant radius of curvature. At least some of the curved edges may have a variable radius of curvature. The shape of the inner edge of each of the one or more reinforcement bands may comprise one or more indentations and/or one or more protrusions or lobes.

The outer edge of each of the one or more reinforcement bands may have a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The outer edge of each of the one or more reinforcement bands may comprise sharp and/or rounded corners. The shape of the outer edge of each of the one or more reinforcement bands comprises straight and/or curved edges. At least some of the curved edges may have a constant radius of curvature. At least some of the curved edges may have a variable radius of curvature. The shape of the outer edge of each of the one or more reinforcement bands may comprise one or more indentations and/or one or more protrusions or lobes.

The lower surface of each of the one or more reinforcement bands may be configured to lie parallel to the skin surface.

According to an aspect there is provided an analyte monitoring system, comprising a reinforcement device as described and an on-body device. The on-body device may comprise the housing; an analyte sensor comprising a first portion arranged within the housing and a second portion configured to be positioned below a skin surface and in contact with a bodily fluid to generate a signal indicative of an analyte level of a user; and sensor electronics arranged within the housing and coupled to the first portion of the analyte sensor, wherein the sensor electronics comprise a processor and wireless communications circuitry. The adhesive patch may be coupled to a first side of the housing and configured to secure the first side of the housing to the skin surface of the user.

The analyte may be one or more of: glucose, ammonium, asparagine, aspartate, cholesterol, choline, creatinine, diaphorase inhibitors, ethanol, glutamate, glycerol, one or more ketones, lactate, lactose, oxygen, phosphate, potassium, pyruvate, and uric acid.

The on-body device may be a glucose monitor.

At least one of the one or more reinforcement bands may comprise an anti-stick layer. The anti-stick layer may be located at or on an upper surface of the one or more reinforcement bands.

The on-body device may comprise an anti-stick layer. The anti-stick layer may be located at or on an upper surface of the on-body device.

The anti-stick layer may comprise a fluoropolymer or silicone.

The anti-stick layer may be a permanent coating.

The anti-stick layer may be formed in or inherent to the material of the reinforcement ring or the on-body device.

The anti-stick layer may be a removable sticker. The removable sticker may comprise a weak adhesive.

Another aspect provides an on-body device for monitoring an analyte, wherein the on-body device comprises an anti-stick layer on an upper surface of the device.

The anti-stick layer may be a coating on the upper surface of the device.

The anti-stick layer may be a removable sticker located on the upper surface of the device.

The anti-stick layer may comprise a fluoropolymer or silicone and/or may be inherent to the material from which the on-body device is constructed or formed, including the on-body device housing.

The on-body device may, or may have the features of, the on-body device of any on-body device discussed herein.

Another aspect provides an adhesive patch for an on-body device, wherein an upper surface of the adhesive patch comprises an anti-stick layer.

The anti-stick layer may be a coating on the upper surface of the adhesive patch.

The anti-stick layer may be a removable sticker located on the upper surface of the adhesive patch.

The anti-stick layer may comprise a fluoropolymer or silicone and/or may be inherent to the material from which the adhesive patch is constructed or formed.

The anti-stick layer may self-adhere to the material from which the adhesive patch is formed.

Additional aspects of adhesive patches are described in main patch and overpatch configurations. In one such aspect, an adhesive patch is provided for use with an on-body device, the adhesive patch comprising: a non-isotropic layer comprising a plurality of elongate tensile resistant regions; and an adhesive layer adhered to the non-isotropic layer.

Non-isotropic patches exhibit beneficial degrees of breathability and durability making them suitable for use on human bodies for extended wear durations.

The non-isotropic layer can have a first axis along a first lateral dimension, a second axis along a second lateral dimension wherein the second axis is orthogonal to the first axis, and a height, and wherein the plurality of elongate tensile resistant regions may be aligned parallel to the first axis and not the second axis.

Deformation along the first axis is more limited by the presence of elongate tensile resistant regions, which resist the tensile force, and which act to maintain the structural integrity of the adhesive patch as a whole while worn on the body and subjected to varying environmental and biological conditions.

The non-isotropic layer may not include a plurality of elongate tensile resistant regions aligned parallel to the second axis.

Each of the plurality of elongate tensile resistant regions can comprise an elongate fiber or thread.

The non-isotropic layer may comprise spunlace fibers.

Each elongate tensile resistant region of the plurality of elongate tensile resistant regions may be adjacent a second region of relatively lower tensile resistance.

Each elongate tensile resistant region of the plurality of elongate tensile resistant regions may comprise relatively more spunlace fibers than an adjacent second region.

Each elongate tensile resistant region of the plurality of elongate tensile resistant regions may comprise a relatively greater density of spunlace fibers than an adjacent second region.

Each elongate tensile resistant region of the plurality of elongate tensile resistant regions may be relatively thicker than an adjacent second region.

The adhesive patch can further comprise a first opening and at least one second opening, wherein the first opening is configured to permit a housing of an on-body device to pass therethrough, and the second opening segments at least one elongate tensile resistant region of the plurality of elongate tensile resistant regions.

The second opening can be oriented transverse to the first axis.

The second opening may be a slit.

The slit may be straight.

The slit may be curved.

The slit may be in a pattern in the form of a wave or sawtooth.

The adhesive patch may comprise a plurality of second openings.

Each second opening of the plurality of second openings may be arranged at a different radial orientation around a center of the adhesive patch.

Each second opening of the plurality of second openings may be arranged at a different radial orientation around a center of the adhesive patch in symmetrical fashion.

Each second opening of the plurality of second openings may be on a first side of the adhesive patch.

In some cases, non-isotropic patches can be prone to exhibit peel away, e.g., around the peripheral edge, while being worn on the skin. Movement of the body in a manner that exhibits repeated tensile force on the non-isotropic patch along the axis of minimal deformation, e.g., the axis parallel to the elongate tensile resistant regions along which deformation is restricted by the elongate tensile resistant regions, can lead to separation of the adhesive and subsequently peel away of the patch from the skin or other underlying object. Placement of one or more openings in the non-isotropic patch in locations that traverse and segment one or more of the elongate tensile resistant regions can increase the deformability of the non-isotropic patch along this restricted axis.

The adhesive patch may comprise a polymeric layer adhered to the non-isotropic layer. The polymeric layer may comprise an elastic polymer.

The added elasticity can assist the patch in returning to an at-rest state after deformation and this can improve the wear duration of the patch.

The polymeric layer may comprise polyester, polyurethane, polyethylene, polyether, elastane (SPANDEX), polyamide, or a combination of two or more thereof.

The polymeric layer may comprise polyester and/or polyurethane.

The polymeric layer may be elastic.

The non-isotropic layer may have a thickness of 11 mils+/−25%.

A thickness of an adhesive layer on a skin-facing side of the non-isotropic layer may be 4 mils+/−25%.

The polymeric layer may have a thickness of 0.8 mils+/−25%.

An adhesive layer between the non-isotropic layer and the polymeric layer may have a thickness of one mil+/−25%.

The non-isotropic layer may have a thickness of 11 mils+/−10%.

A thickness of an adhesive layer on a skin-facing side of the non-isotropic layer may be 4 mils+/−10%.

The polymeric layer may have a thickness of 0.8 mils+/−10%.

An adhesive layer between the non-isotropic layer and the polymeric layer may have a thickness of one mil+/−10%.

The adhesive patch can include an adhesive layer on an upper surface configured to adhere over at least a portion of an on-body device comprising a housing and a different adhesive patch.

The adhesive patch may be configured as a main patch adhered to an underside of a housing of an on-body device.

The adhesive patch may be configured as an overpatch.

The on-body device may be configured for analyte monitoring.

The adhesive patch can be configured for application over an on-body device comprising a housing adhered to a different adhesive patch.

In other aspects, on-body devices, analyte monitoring systems, and methods of application are provided where each is in relation to the adhesive patches described.

In an aspect, an on-body device configured to monitor an analyte of a body is provided, comprising: an on-body device housing comprising electronics configured to interface with an analyte sensor; and an adhesive patch adhered to the on-body device housing, wherein the adhesive patch is configured in accordance with the adhesive patch as described herein.

In an aspect, an analyte monitoring system is provided comprising: an on-body device comprising a housing comprising electronics configured to interface with an analyte sensor and a first adhesive patch adhered to the on-body device housing; and a second adhesive patch configured to be applied over the housing and the first adhesive patch, wherein either or both of the first adhesive patch and second adhesive patch are configured in accordance with the adhesive patch as described herein.

In an aspect, a method of applying an on-body device to a body is provided, comprising: applying an adhesive patch to skin of the body, wherein the adhesive patch is coupled to an on-body device housing, wherein the adhesive patch is configured in accordance with the adhesive patch as described herein.

In an aspect, a method of applying an overpatch to a body is provided, comprising: applying the overpatch over an on-body device and onto skin of the body, wherein the overpatch is configured in accordance with the adhesive patch as described herein.

In another aspect, an overpatch assembly is provided comprising: an overpatch comprising a backing and an adhesive layer, wherein the overpatch has an opening therethrough configured to receive an on-body device; and a removable first liner adhered to the adhesive layer.

The overpatch assembly may comprise a support structure in the opening of the overpatch, the support structure having a recess configured to receive the on-body device.

The housing of the on-body device may have a peripheral shape that is not rotationally symmetric, and the recess of the support structure may have a peripheral shape corresponding to the peripheral shape of the on-body device.

The support structure may comprise a first physical alignment feature adapted to interface with a second physical alignment feature of the on-body device.

The first and second physical alignment features may interface in a male and female configuration.

The support structure may comprise at least one aperture or open region adapted to allow moisture to vent from the on-body device through the support structure.

The support structure may comprise a plurality of apertures or open regions adapted to allow moisture to vent from a channel terminations and/or vent outlets of the on-body device and through the support structure.

The support structure may comprise a standoff, or a plurality of standoffs, that creates a gap between the on-body device housing and the support structure through which moisture from the volume around the on-body device can vent out.

The first liner may comprise a pull tab portion.

The pull tab portion may extend into the opening of the overpatch.

The overpatch assembly may comprise at least one secondary adhesive liner adhered to the adhesive layer.

The at least one secondary adhesive liner may comprise a pull tab portion.

The overpatch assembly may comprise two secondary adhesive liners, each comprising a pull tab portion. The two secondary adhesive liners may each be adhered to the adhesive layer in a folded over configuration.

The overpatch assembly may not include a separate and discrete removable stiffener layer.

The overpatch assembly may comprise a removable stiffener layer.

The stiffener layer may be adhesively coupled to an upper liner.

The stiffener layer reduces the likelihood that the overpatch assembly will bend, droop, or

collapse during application and thus reduces the likelihood that overpatch assembly will be adhered to the user's skin in an unintended manner.

The adhesive layer may be a first adhesive layer, and the overpatch assembly may further comprise a stiffener layer and a second adhesive layer adhering the stiffener layer to the backing, wherein a degree of adhesion of the stiffener layer to the second adhesive layer, a degree of adhesion of the second adhesive layer to the backing, and a degree of adhesion of the backing to the first adhesive layer may all be greater than a degree of adhesion of the removable first liner to the first adhesive layer. At least one of the degree of adhesion of the stiffener layer to the second adhesive layer and the degree of adhesion of the second adhesive layer to the backing may be less than the degree of adhesion of the backing to the first adhesive layer and the degree of adhesion of the first adhesive layer to skin. The overpatch assembly may further comprise an upper liner and a third adhesive layer that couples the upper liner to the stiffener layer, wherein a degree of adhesion of the upper liner to the third adhesive layer and a degree of adhesion of the third adhesive layer to the stiffener layer may be greater than at least one of the degree of adhesion of the stiffener layer to the second adhesive layer and the degree of adhesion of the second adhesive layer to the backing.

The support structure may comprise a first retainer structure, a sidewall, and a second retainer structure.

The first retainer structure may be configured for placement against a first side of the on-body device and the second retainer structure may be configured for placement against skin of the wearer.

The first retainer structure may comprise an opening.

The first retainer structure may be configured as a lip.

The second retainer structure may comprise at least one extension section located adjacent to at least one gap region.

The second retainer structure may comprise a plurality of extension sections with a plurality of gap regions located therebetween.

The first liner may cover a first portion of the adhesive layer and the second retainer structure.

The first portion of the adhesive layer may be adhered to the skin through at least one gap region.

The adhesive layer may be a skin adhesive layer, wherein the removable first liner may be folded, and wherein the overpatch assembly may further comprise a removable second liner that is folded and adhered to the skin adhesive layer.

A first portion of the skin adhesive layer may be exposed with the first and second folded liners adhered in place over the skin adhesive layer.

The exposure of the adhesive layer provides a sticky surface that a user can use to attach overpatch assembly to the skin of the on-body device wearer.

The first portion of the skin adhesive layer may be exposed in a gap between the first and second folded liners in place over the skin adhesive layer.

The gap may be present across an entire width of the skin adhesive layer.

At least one of the first and second folded liners may comprise at least one open region through which the skin adhesive layer is exposed.

At least one open region may be intersected by a crease in the at least one of the first and second folded liners.

The backing and skin adhesive layer may have an interior space configured to configured to allow passage of a housing of an on-body device therethrough.

The interior space may have a first perimeter edge, wherein a portion of the removable first liner that is adhered to the skin adhesive layer may have a second perimeter edge adjacent to and not aligned with the first perimeter edge.

A portion of the removable second liner that is adhered to the skin adhesive layer may have a third perimeter edge adjacent to and not aligned with the first perimeter edge.

The backing and skin adhesive layer may have an edge along the interior space and the removable first and second liners may not be adhered to at least a portion of the edge.

The removable first and second liners may not be adhered to any portion of the edge.

The removable first liner may comprise a first enclosed open region when in an unfolded state. The removable second liner may comprise a second enclosed open region when in an unfolded state.

The first and second enclosed open regions may be asymmetrical such that when the removable first and second liners are in a folded state, both folded portions of each of the removable first and second liners are adhered to the skin adhesive layer.

The removable first and second liners may be folded such that a first portion of each of the removable first and second liners is adhered directly to the skin adhesive layer and a second portion of each of the removable first and second liners is adhered to the skin adhesive layer through an open region of the first portion.

The first and second enclosed open regions may each be wider than the interior space.

The first and second enclosed open regions may each be wider than the interior space in in all directions of a two coordinate plane.

The first and second enclosed open regions may have a symmetric shape.

The first and second enclosed open regions may have an asymmetric shape.

The first and second enclosed open regions may each have at least one side that is tapered.

The first and second enclosed open regions may permit at least a portion of the skin adhesive layer to be exposed when the removable first and second liners are folded and in contact with the skin adhesive layer.

The overpatch may comprise at least one layer that is a non-isotropic layer.

The backing may comprise the non-isotropic layer and wherein the adhesive layer is adhered to the non-isotropic layer.

The non-isotropic layer may have a first axis along a first lateral dimension, a second axis along a second lateral dimension wherein the second axis is orthogonal to the first axis, and a height, and wherein the non-isotropic layer may comprise a plurality of elongate tensile resistant regions aligned parallel to the first axis and not the second axis. Each of the plurality of elongate tensile resistant regions may comprise an elongate fiber or thread.

The non-isotropic layer may not include a plurality of elongate tensile resistant regions aligned parallel to the second axis.

The non-isotropic layer may comprise spunlace fibers.

Each elongate tensile resistant region of the plurality of elongate tensile resistant regions may be adjacent a second region of relatively lower tensile resistance.

Each elongate tensile resistant region of the plurality of elongate tensile resistant regions may comprise relatively more spunlace fibers than an adjacent second region.

Each elongate tensile resistant region of the plurality of elongate tensile resistant regions may comprise a relatively greater density of spunlace fibers than an adjacent second region.

Each elongate tensile resistant region of the plurality of elongate tensile resistant regions may have relatively thicker than an adjacent second region.

The overpatch assembly may further comprise a first opening and at least one second opening, wherein the first opening is configured to permit a housing of an on-body device to pass therethrough, and the second opening segments at least one elongate tensile resistant region of the plurality of elongate tensile resistant regions.

The second opening may be oriented transverse to the first axis. The second opening may be a slit. The slit may be straight. The slit may be curved. The slit may have a pattern in the form of a wave or sawtooth.

The overpatch assembly may comprise a plurality of the second openings.

Each second opening of the plurality of second openings may be arranged at a different radial orientation around a center of the adhesive patch.

Each second opening of the plurality of second openings may be arranged at a different radial orientation around a center of the adhesive patch in symmetrical fashion.

Each second opening of the plurality of second openings may be on a first side of the adhesive patch.

The non-isotropic layer may comprise a polymeric material.

The backing further may comprise a polymeric layer adhered to a non-skin-facing side of the non-isotropic layer.

The polymeric layer may comprise polyester, polyurethane, polyethylene, polyether, elastane, polyamide, or a combination of two or more thereof.

The polymeric layer may comprise polyester and/or polyurethane.

The polymeric layer may be elastic.

The non-isotropic layer may have a thickness of 11 mils+/−25%.

A thickness of an adhesive layer on a skin-facing side of the non-isotropic layer may be 4 mils+/−25%.

The polymeric layer may have a thickness of 0.8 mils+/−25%.

An adhesive layer between the non-isotropic layer and the polymeric layer may have a thickness of one mil+/−25%.

The non-isotropic layer may have a thickness of 11 mils+/−10%.

A thickness of an adhesive layer on a skin-facing side of the non-isotropic layer may be 4 mils+/−10%.

The polymeric layer may have a thickness of 0.8 mils+/−10%.

An adhesive layer between the non-isotropic layer and the polymeric layer may have a thickness of one mil+/−10%.

The on-body device may be configured for analyte monitoring.

In an aspect, a product is provided, comprising: an overpatch assembly as described herein; and a package in which the overpatch assembly is contained, wherein the package comprises a liner surface adhered to an exposed portion of adhesive of the overpatch assembly, wherein the liner surface is secured to the package.

The package may further comprise a conductive element.

The conductive element may be a metallic strip.

The conductive element may be configured to conduct an induced current.

In another aspect, a product is provided, the product comprising: an overpatch assembly; and a package in which the overpatch assembly is contained, wherein the package comprises a conductive element.

The conductive element may be secured to the package.

The conductive element may be secured to the overpatch assembly.

The conductive element may be a metallic strip.

The conductive element may be configured to conduct an induced current.

The package may further comprise a liner surface adhered to an exposed portion of adhesive of the overpatch assembly, wherein the liner surface is secured to the package.

The product may further comprise a kit package comprising: the overpatch assembly, the package having the overpatch assembly therein; and at least one of an on-body device or an inserter for the on-body device.

In another aspect, a method of testing a kit package is provided, the method comprising: applying an electromagnetic field to the kit package, wherein the kit package comprises an overpatch package comprising an overpatch, wherein a conductive element is present in the overpatch package or the overpatch, and wherein the kit package further comprises at least one of an on-body device or an inserter for the on-body device; and sensing a magnitude of an induced electrical current within the kit package.

The method may further comprise verifying that the overpatch package or the overpatch is present within the kit package.

The conductive element may be configured to conduct an induced current.

The conductive element may be configured as a metallic strip secured to the overpatch package.

In another aspect, a method of applying an overpatch to a wearer of an on-body device is provided, the method comprising: positioning an overpatch assembly over at least a portion of the on-body device while a first portion of an adhesive layer of the overpatch is exposed and a second portion of the adhesive layer is unexposed; and adhering the first portion of the adhesive layer to skin of the wearer while the housing of the on-body device is within the opening and the second portion of the adhesive layer is unexposed.

Adhering the first portion of the adhesive layer to skin of the wearer while the second portion of the adhesive layer is unexposed may comprise manually pressing a side of the overpatch assembly opposite to the first portion of the adhesive layer to adhere the first portion of the adhesive layer to the skin.

The method may further comprise removing, by the user, a first liner to expose the first portion of the adhesive layer prior to positioning the overpatch assembly over the at least a portion of the on-body device.

The first liner may be secured in an interior of a package of the overpatch assembly.

Removing, by the user, the first liner to expose the first portion of the adhesive layer may be accomplished by removing the overpatch assembly from within the package.

The method may further comprise removing at least one folded liner to expose at least a part of the second portion of the adhesive layer after adhering the first portion of the adhesive layer to skin of the wearer.

The method may further comprise adhering the exposed at least a part of the second portion of the adhesive layer to the skin.

Adhering the exposed at least a part of the second portion of the adhesive layer to the skin may comprise manually pressing a side of the overpatch assembly opposite to the at least a part of the second portion of the adhesive layer to adhere the at least a part of the second portion of the adhesive layer to the skin.

The method may further comprise removing a first folded liner to expose a first section of the second portion of the adhesive layer after adhering the first portion of the adhesive layer to skin of the wearer.

The overpatch assembly may comprise an overpatch having the adhesive layer thereon, wherein the overpatch has an interior space, wherein the first folded liner has an enclosed open region when in an unfolded state, and wherein the first folded liner is configured such that the last portion of a perimeter edge of the enclosed open region to be removed from the adhesive layer is removed at a location of the adhesive layer spaced away from a perimeter of the interior space of the overpatch.

The method may further comprise adhering the exposed first section of the second portion of the adhesive layer to the skin.

Adhering the exposed first section of the second portion of the adhesive layer to the skin may comprise manually pressing a side of the overpatch assembly opposite to the first section of the second portion of the adhesive layer to adhere the first section of the second portion of the adhesive layer to the skin.

Removing the first folded liner may comprise concurrently removing and unfolding the first folded liner by pulling on the first folded liner.

The method may further comprise removing a second folded liner to expose a second section of the second portion of the adhesive layer after adhering the first section of the second portion of the adhesive layer to skin of the wearer.

The second folded liner may have an enclosed open region when in an unfolded state, and the second folded liner may be configured such that the last portion of a perimeter edge of the enclosed open region to be removed from the adhesive layer is removed at a location of the adhesive layer spaced away from a perimeter of the interior space of the overpatch.

The method may further comprise adhering the exposed second section of the second portion of the adhesive layer to the skin.

Adhering the exposed second section of the second portion of the adhesive layer to the skin may comprise manually pressing a side of the overpatch assembly opposite to the second section of the second portion of the adhesive layer to adhere the second section of the second portion of the adhesive layer to the skin.

Removing the second folded liner may comprise concurrently removing and unfolding the second folded liner by pulling on the second secondary adhesive liner.

The overpatch assembly may not include a removable stiffener layer distinct from the overpatch and the at least one removable folded liner.

The method may further comprise removing a stiffener layer from the overpatch after fully adhering the overpatch to skin of the wearer of the on-body device.

The overpatch may comprise a backing layer coupled with the adhesive layer.

The method may further comprise repositioning the overpatch assembly prior to removing the at least one folded liner.

Repositioning the overpatch assembly prior to removing at least one folded liner may comprise un-adhering the first portion of the adhesive layer from the skin, moving the overpatch assembly, and then re-adhering the first portion of the adhesive layer to the skin.

In another aspect, a method of applying an overpatch to a wearer of an on-body device is provided, the method comprising: positioning a support structure of an overpatch assembly on a housing of the on-body device while a first portion of an adhesive layer of the overpatch is exposed and a second portion of the adhesive layer is unexposed; and adhering the first portion of the adhesive layer to skin of the wearer while the support structure is on the housing and the second portion of the adhesive layer is unexposed.

Positioning the support structure of the overpatch assembly on the housing of the on-body device may comprise positioning the support structure of the overpatch in contact with the housing of the on-body device.

Positioning the support structure of the overpatch assembly on the housing of the on-body device may comprise aligning the support structure over the housing of the on-body device.

The housing of the on-body device may have a peripheral shape that is not rotationally symmetric, and aligning the support structure over the housing of the on-body device may comprise aligning the peripheral shape of the housing of the on-body device with a corresponding peripheral shape of a recess within the support structure.

Aligning the support structure over the housing of the on-body device may comprise aligning a physical alignment feature of the housing of the on-body device with a corresponding inverse physical alignment feature of the support structure.

The on-body device may comprise a plurality of side-facing channel terminations or vent outlets and the support structure may comprise at least one aperture, wherein aligning the support structure over the housing of the on-body device is such that the plurality of side-facing channel terminations or vent outlets are aligned with the at least one aperture when the support structure is in a final resting position over the on-body device.

Positioning the support structure of the overpatch assembly on the housing of the on-body device may comprise sliding the support structure of the overpatch over the housing of the on-body device such that the housing of the on-body device is received within an interior space of the support structure.

Adhering the first portion of the adhesive layer to skin of the wearer while the support structure is on the housing and the second portion of the adhesive layer is unexposed may comprise manually pressing a side of the overpatch assembly opposite to the first portion of the adhesive layer to adhere the first portion of the adhesive layer to the skin.

The method may further comprise removing, by the user, a first skin adhesive liner to expose the first portion of the adhesive layer prior to positioning the support structure of the overpatch assembly on the housing of the on-body device.

The method may further comprise removing at least one secondary adhesive liner to expose the second portion of the adhesive layer after adhering the first portion of the adhesive layer to skin of the wearer.

The method may comprise adhering the exposed second portion of the adhesive layer to the skin.

Adhering the exposed second portion of the adhesive layer to the skin may comprise manually pressing a side of the overpatch assembly opposite to the second portion of the adhesive layer to adhere the second portion of the adhesive layer to the skin.

The method may further comprise removing a first secondary adhesive liner to expose a first section of the second portion of the adhesive layer after adhering the first portion of the adhesive layer to skin of the wearer.

The method may further comprise adhering the exposed first section of the second portion of the adhesive layer to the skin.

Adhering the exposed first section of the second portion of the adhesive layer to the skin may comprise manually pressing a side of the overpatch assembly opposite to the first section of the second portion of the adhesive layer to adhere the first section of the second portion of the adhesive layer to the skin.

Removing the first secondary adhesive liner may comprise concurrently removing and unfolding the first secondary adhesive liner by pulling on the first secondary adhesive liner.

The method may further comprise removing a second secondary adhesive liner to expose a second section of the second portion of the adhesive layer after adhering the first section of the second portion of the adhesive layer to skin of the wearer.

The method may further comprise adhering the exposed second section of the second portion of the adhesive layer to the skin.

Adhering the exposed second section of the second portion of the adhesive layer to the skin may comprise manually pressing a side of the overpatch assembly opposite to the second section of the second portion of the adhesive layer to adhere the second section of the second portion of the adhesive layer to the skin.

Removing the second secondary adhesive liner may comprise concurrently removing and unfolding the second secondary adhesive liner by pulling on the second secondary adhesive liner.

The method may further comprise removing a stiffener unit from the overpatch assembly. The stiffener layer may be removed by pulling a pull tab of the stiffener unit.

The stiffener unit may comprise an upper liner coupled to a stiffener layer.

Removal of the stiffener unit may leave an overpatch of the overpatch assembly in place on the skin over at least a portion of the on-body device.

The overpatch may comprise a backing layer coupled with the adhesive layer.

The first portion of the adhesive layer may be immediately adjacent the support structure.

The support structure may comprise a first retainer structure, a sidewall, and a second retainer structure.

The first retainer structure may be configured for placement against a first side of the on-body device, and the second retainer structure may be configured for placement against skin of the wearer.

The first retainer structure may comprise an opening.

The first retainer structure may be configured as a lip.

Positioning the support structure of the overpatch assembly on the housing of the on-body device may comprise sliding the support structure of the overpatch over the housing of the on-body device until the housing contacts the upper retainer structure.

The second retainer structure may comprise at least one extension section located adjacent to at least one gap region.

The first portion of the adhesive layer may be in a gap region adjacent an extension section of the support structure.

The second retainer structure may comprise a plurality of extension sections with a plurality of gap regions located therebetween.

The first portion of the adhesive layer may be non-contiguous.

The first portion of the adhesive layer may be adhered to the skin through the plurality of gap regions.

The method may comprise attaching the support structure to the first portion of the adhesive layer prior to positioning the support structure of the overpatch assembly on the housing of the on-body device.

The method may comprise repositioning the overpatch assembly prior to removing at least one secondary adhesive liner.

Repositioning the overpatch assembly prior to removing at least one secondary adhesive liner may comprise un-adhering the first portion of the adhesive layer from the skin, moving the overpatch assembly, and then re-adhering the first portion of the adhesive layer to the skin.

In another aspect, an adhesive patch for use with an on-body device is provided, comprising: a first adhesive layer; a second adhesive layer; a first structural layer having a first side adhered to the first adhesive layer and a second side adhered to the second adhesive layer; and a second structural layer having a first side adhered to the second adhesive layer. The adhesive patch can exhibit duration and breathability characteristics that make it suitable for extended wear on the body as part of an on-body device.

The first adhesive layer may function as a skin adhesive layer.

The second adhesive layer may function to adhere first structural layer to the second structural layer.

The first structural layer may comprise a polymeric or plastic material.

The first structural layer may comprise a polyester, a polyurethane (PU), a polyethylene, a polyether, an elastane, a polyamide, or a combination of two or more thereof.

The first structural layer may comprise a polyethylene terephthalate (PET).

The first structural layer may comprise a polyurethane (PU).

The first structural layer may be an isotropic layer.

The first structural layer may be a non-isotropic layer.

The second structural layer may comprise a polymeric or plastic material.

The second structural layer may comprise a polyester, a polyurethane (PU), a polyethylene, a polyether, an elastane, a polyamide, or a combination of two or more thereof.

The second structural layer may comprise a polyethylene terephthalate (PET).

The second structural layer may comprise a polyurethane (PU).

The second structural layer may be an isotropic layer.

The second structural layer may be a non-isotropic layer.

The first structural layer may be a non-isotropic spunlace polymer and the second structural layer may comprise polyurethane.

The first adhesive layer may comprise a pressure sensitive adhesive.

The first adhesive layer may comprise an acrylic adhesive.

The first adhesive layer may comprise at least one of a silicone adhesive, a hydrocarbon adhesive, a polyurethane adhesive, a natural rubber adhesive, and/or a synthetic rubber adhesive.

The second adhesive layer may comprise a pressure sensitive adhesive.

The second adhesive layer may comprise an acrylic adhesive.

The second adhesive layer may comprise at least one of a silicone adhesive, a hydrocarbon adhesive, a polyurethane adhesive, a natural rubber adhesive, and/or a synthetic rubber adhesive.

The adhesive patch may exhibit a moisture vapor transmission rate (MVTR) of 115 grams/square meter/day or higher.

The adhesive patch may exhibit a moisture vapor transmission rate (MVTR) of 175 grams/square meter/day or higher.

The adhesive patch may exhibit a moisture vapor transmission rate (MVTR) of 200 grams/square meter/day or higher.

The first structural layer may have a thickness of 11 mils+/−25%.

The first adhesive layer may have a thickness of 4 mils+/−25%.

The second structural layer may have a thickness of 0.8 mils+/−25%.

The second adhesive layer may have a thickness of one mil+/−25%.

The first structural layer may have a thickness of 11 mils+/−10%.

The first adhesive layer may have a thickness of 4 mils+/−10%.

The second structural layer may have a thickness of 0.8 mils+/−10%.

The second adhesive layer may have a thickness of one mil+/−10%.

The third adhesive layer may adhere the second structural layer to a housing of an on-body device.

The third adhesive layer may comprise a pressure sensitive adhesive.

The third adhesive layer may comprise an acrylic adhesive.

The third adhesive layer may comprise at least one of a silicone adhesive, a hydrocarbon adhesive, a polyurethane adhesive, a natural rubber adhesive, and/or a synthetic rubber adhesive.

The adhesive patch may be configured as a main adhesive patch for an on-body device comprising a housing.

The adhesive patch may be configured as an overpatch for at least a portion of an on-body device comprising a housing and a main adhesive patch.

In another aspect, an on-body device configured to monitor an analyte of a body is provided that comprises: an on-body device housing comprising electronics configured to interface with an analyte sensor; an adhesive patch adhered to the on-body device housing, wherein the adhesive patch is configured in accordance with any of the aforementioned features of the adhesive patch.

In another aspect, an analyte monitoring system is provided, comprising: an on-body device comprising a housing comprising electronics configured to interface with an analyte sensor and a first adhesive patch adhered to the on-body device housing; and a second adhesive patch configured to be applied over the housing and the first adhesive patch, wherein either or both of the first adhesive patch and second adhesive patch are configured in accordance with any of the aforementioned features of the adhesive patch.

In another aspect, a method of applying an on-body device to a body is provided, comprising: applying an adhesive patch to skin of the body, wherein the adhesive patch is coupled to an on-body device housing, wherein the adhesive patch is configured in accordance with any of the aforementioned features of the adhesive patch.

In another aspect, an on-body device for monitoring an analyte level of a user is provided, the on-body device comprising: a housing; sensor electronics, wherein the housing is configured to retain the sensor electronics; a sensor; and an adhesive patch attached to the housing and configured to attach the housing to skin of the user.

Any of the aforementioned on-body devices may comprise an injection molded housing, a one-shot molded housing, an over-molded housing, a housing with at least two parts, and/or a housing comprising a first part and a second part assembled together with sensor electronics arranged between.

Any of the aforementioned on-body devices may comprise a housing with a recess configured to receive a sensor connector, wherein the recess is arranged in a top or a bottom of the housing. The sensor connector may be coupled with an analyte sensor. Filling material may be in the recess.

Any of the aforementioned on-body devices may comprise a housing with a recess configured to receive sensor electronics, where the recess is arranged in a top or a bottom of the housing.

The aforementioned aspects of this Summary section can be implemented with on-body devices and/or analyte monitoring systems having the following arrangements. In other words, the features of the on-body devices and/or analyte monitoring systems in the foregoing aspects may be incorporated into any of the following arrangements. Additionally, any of the following arrangements may include one or more of the adhesive patches and/or reinforcement devices as described in the foregoing aspects.

In a first arrangement of the on-body device, the housing may comprise a first part and a second part attached together, wherein the housing may be water-resistant; the sensor electronics may comprise: processing electronics present on a semiconductor chip; and wireless communications electronics present on the semiconductor chip and configured to communicate according to a Bluetooth Low Energy protocol; the sensor may have a flexible substrate, and the sensor may comprise: a first portion retained within the housing and comprising three contacts configured to be in electrical contact with the sensor electronics; and a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion.

The on-body device may further comprise a temperature sensor present on a side of the first part of the housing adhered to the adhesive patch, wherein: the sensor electronics may be coupled to a printed circuit board, the sensor electronics may further comprise a coin cell battery and a magnetic switch; the sensor may comprise a fold; the first part of the housing may comprise a plurality of recesses adapted to interface with inserter projections; and the adhesive patch may protrude beyond a peripheral boundary of the housing.

In a second arrangement, an analyte monitoring system is provided, comprising: an on-body device comprising a sensor; and an inserter comprising: a removable cap; a sharp; a retraction spring; a housing; a handle configured to move from an initial position to an advanced position with respect to the housing to advance the sharp into skin of a recipient's body and concurrently advance the on-body device into position on the recipient's body, wherein the inserter is configured to automatically retract the sharp with the retraction spring.

The on-body device may be housed within an interior space of the inserter, the interior space being sealed by a removable cap coupled with the handle and/or housing of the inserter, and the sharp and the sensor may be maintained in a sterile environment by a sheath of the removable cap. The sharp may comprise: a distal tip portion with a V-shaped portion having a first angle of 20 to 60 degrees; a proximal shaft portion comprising a first sidewall, a floor, and a second sidewall together providing a U-shape or a C-shape cross-sectional profile; and a transition portion between the distal tip portion and the proximal shaft portion where the first sidewall and the second sidewall each have a height from the floor that is sloped at a second angle of 7 to 35 degrees. The first angle may be between 45 and 55 degrees and the second angle may be between 20 and 30 degrees. The analyte monitoring system may further comprise a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

In a third arrangement, an analyte monitoring system is provided, comprising: an on-body device comprising a sensor; and a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

In the second and third arrangements, the analyte monitoring system may further comprise an overpatch. The overpatch may be configured in accordance with the overpatches described in the foregoing aspects.

In the second and third arrangements, the on-body device and the sensor may be in accordance with those of the first arrangement.

In a fourth arrangement of the on-body device, the housing may be configured to be water-resistant; the electronics may comprise: sensor interface electronics; wireless communications electronics configured to communicate according to a Bluetooth Low Energy protocol; the sensor may be configured to detect or measure an analyte with a working electrode and a counter/reference electrode, and the sensor may comprise: a central substrate configured as a wire; and a plurality of layers on the central substrate, the plurality of layers comprising a diffusion limiting layer, an enzyme layer, and an interferent layer.

The sensor electronics may further comprise: a first antenna configured for communication according to the Bluetooth Low Energy protocol; a second antenna configured for communication according to a Near Field Communication protocol; a coin cell battery; and a magnetic switch. The on-body device may further be provided such that the sensor is in a bent configuration, the housing is a molded housing comprising a plurality of first recesses configured to engagement with a plurality of first projections of an inserter to hold the on-body device to the inserter and comprising a second recess configured to interface with a second projection of the inserter to inhibit rotation of the on-body device, and the adhesive patch protrudes beyond a peripheral boundary of the housing.

In a fifth arrangement, an analyte monitoring system is provided, comprising: an on-body device comprising a sensor; and an inserter configured to automatically advance a sharp and move the on-body device and configured to automatically retract the sharp, the inserter comprising: a removable cap; the sharp; a first spring configured to advance the sharp; a second spring configured to retract the sharp; a housing comprising an actuator configured to initiate advancement of the sharp.

The on-body device may be housed within an interior space of the inserter, the interior space being sealed by the removable cap. The sharp may comprise a shaft comprising a first sidewall, a floor, and a second sidewall together providing a U-shape or a C-shape cross-sectional profile, the shaft having a bevelled distal tip having an angle of 20 to 45 degrees. The analyte monitoring system may further comprise: a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

In a sixth arrangement, an analyte monitoring system is provided, comprising: an on-body device comprising a sensor; and a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

The analyte monitoring systems of the fourth and fifth arrangements may further comprise an overpatch. The overpatch may be configured in accordance with the overpatches described in the foregoing aspects.

In the fifth and sixth arrangements, the on-body device and the sensor may be in accordance with those of the fourth arrangement.

In a seventh arrangement of the on-body device, the housing may comprise a first part and a second part attached together, wherein the housing may be water-resistant; the electronics may comprise:

processing electronics present on a semiconductor chip; and wireless communications electronics present on the semiconductor chip and configured to communicate according to a Bluetooth Low Energy protocol; the sensor may have a flexible substrate, and the sensor may comprise: a first portion retained within the housing and comprising three contacts configured to be in electrical contact with the sensor electronics; and a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion.

The on-body device may be provided such that: the sensor electronics may further comprise a coin cell battery, a magnetic switch, a printed circuit board, and an antenna, wherein the antenna is configured to communicate according to the Bluetooth Low Energy protocol, and wherein the antenna is mounted on the housing and is electrically connected to the wireless communications electronics on the printed circuit board; the housing comprises a plurality of recesses adapted to interface with inserter projections; and the adhesive patch protrudes beyond a peripheral boundary of the housing. The coin cell battery may be electrically connected to the printed circuit board and may be held adjacent to the printed circuit board such that the coin cell battery and the printed circuit board are both in a same plane.

In an eighth arrangement, an analyte monitoring system is provided, comprising: an on-body device comprising a sensor; and an inserter configured to automatically advance a sharp, move the on-body device, and automatically retract the sharp, the inserter comprising: a removable cap; the sharp; a first spring configured to advance the sharp; a second spring configured to retract the sharp; and a housing comprising an actuator configured to initiate advancement of the sharp.

The on-body device may be housed within an interior space of the inserter, the interior space being sealed by a removable cap coupled with the housing of the inserter, and wherein the sharp and the sensor may be maintained in a sterile environment by a sheath of the removable cap. The sharp may comprise: a distal tip portion with a V-shaped portion having a first angle of 20 to 60 degrees; a proximal shaft portion comprising a first sidewall, a floor, and a second sidewall together providing a U-shape or a C-shape cross-sectional profile; and a transition portion between the distal tip portion and the proximal shaft portion where the first sidewall and the second sidewall each have a height from the floor that is sloped at a second angle of 7 to 35 degrees. The first angle may be between 28 and 38 degrees and the second angle may be between 18 and 28 degrees. The analyte monitoring system may further comprise: a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

In a ninth arrangement, an analyte monitoring system is provided, comprising: an on-body device comprising a sensor; and a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

The analyte monitoring systems of the eighth and ninth arrangements may further comprise an overpatch. The overpatch may be configured in accordance with the overpatches described herein.

In the eighth and ninth arrangements, the on-body device and the sensor may be in accordance with those of the seventh arrangement.

In a tenth arrangement of the on-body device, the housing may comprise a first part and a second part attached together; the electronics may comprise: processing electronics present on a semiconductor chip; and wireless communications electronics present on the semiconductor chip and configured to communicate according to a Bluetooth Low Energy protocol; a sensor connector; the sensor may have a flexible substrate, and the sensor may be coupled with the sensor connector and comprise: a first portion retained within the housing and comprising three contacts configured to be in electrical contact with the sensor electronics; and a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion, wherein the housing may comprise a recess configured to receive the sensor connector, and wherein the housing may be configured to be water-resistant when the sensor connector is received within the recess.

The on-body device may be provided such that: the sensor electronics further comprise a coin cell battery, a magnetic switch, a printed circuit board, and an antenna, wherein the antenna is configured to communicate according to the Bluetooth Low Energy protocol; the housing comprises a plurality of recesses adapted to interface with inserter projections; and the adhesive patch protrudes beyond a peripheral boundary of the housing. The coin cell battery may be electrically connected to the printed circuit board and may be held adjacent to the printed circuit board such that the coin cell battery and the printed circuit board are both in a same plane.

In an eleventh arrangement, an analyte monitoring system is provided, comprising: an on-body device comprising a recess; and an inserter configured to hold the on-body device and automatically advance a sensor connector and a sharp into the recess of the on-body device, wherein the sensor connector is coupled with a sensor, wherein the inserter is configured to automatically retract the sharp, the inserter comprising: the sharp; a first spring configured to advance the sharp; a second spring configured to retract the sharp; and a housing comprising an actuator configured to initiate advancement of the sharp.

The sharp may comprise: a distal tip portion with a V-shaped portion having a first angle of 20 to 60 degrees; a proximal shaft portion comprising a first sidewall, a floor, and a second sidewall together providing a U-shape or a C-shape cross-sectional profile; and a transition portion between the distal tip portion and the proximal shaft portion where the first sidewall and the second sidewall each have a height from the floor that is sloped at a second angle of 7 to 35 degrees. The first angle may be between 30 and 40 degrees and the second angle may be between 13 and 23 degrees. The analyte monitoring system may further comprise: a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

In a twelfth arrangement, an analyte monitoring system is provided, comprising: an on-body device comprising a sensor; and a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

The analyte monitoring systems of the eleventh and twelfth arrangements may further comprise an overpatch. The overpatch may be configured in accordance with the overpatches described herein.

In the eleventh and twelfth arrangements, the on-body device and the sensor may be in accordance with those of the tenth arrangement.

In a thirteenth arrangement, an on-body device for monitoring an analyte level of a user is provided, comprising: a housing comprising a first part and a second part attached together; sensor electronics, wherein the housing is configured to retain the sensor electronics, the sensor electronics comprising: processing electronics present on a semiconductor chip; and wireless communications electronics present on the semiconductor chip and configured to communicate according to a Bluetooth Low Energy protocol; a sensor having a flexible substrate, the sensor coupled with the sensor connector and comprising: a first portion retained within the housing and comprising three contacts configured to be in electrical contact with the sensor electronics; and a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion; and an adhesive patch attached to the housing and configured to attach the housing to skin of the user, wherein the housing is configured to be water-resistant.

The on-body device may be provided such that: the sensor electronics further comprise a coin cell battery, a magnetic switch, a printed circuit board, and an antenna, wherein the antenna is configured to communicate according to the Bluetooth Low Energy protocol; the housing comprises a recess adapted to interface with an inserter projection; the adhesive patch protrudes beyond a peripheral boundary of the housing; the sensor comprises a fold; and the adhesive patch is attached to the first part of the housing. The coin cell battery may be electrically connected to the printed circuit board and may be held adjacent to the printed circuit board such that the coin cell battery and the printed circuit board are both in a same plane.

In a fourteenth arrangement, an analyte monitoring system is provided, comprising: an on-body device comprising a sensor; and an inserter configured to automatically advance a sharp, move the on-body device, and automatically retract the sharp, the inserter comprising: a removable cap; the sharp; a first spring configured to advance the sharp; a second spring configured to retract the sharp; a housing comprising an actuator configured to initiate advancement of the sharp. The on-body device may be housed within an interior space of the inserter, the interior space being sealed by the removable cap coupled with the housing of the inserter, and wherein the sharp and the sensor are maintained in a sterile environment by a sheath of the removable cap. The sharp may comprise a distal tip portion with a V-shaped portion having a first angle of 20 to 60 degrees; the sharp comprises a proximal shaft portion comprising a first sidewall, a floor, and a second sidewall together providing a U-shape or a C-shape cross-sectional profile; the sharp comprises a transition portion between the distal tip portion and the proximal shaft portion where the first sidewall and the second sidewall each have a height from the floor that is sloped at a second angle of 7 to 35 degrees. The first angle may be between 31 and 41 degrees and the second angle is between 13 and 23 degrees. The analyte monitoring system may further comprise: a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

In a fifteenth arrangement, an analyte monitoring system is provided, comprising: an on-body device comprising a sensor; and a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

The analyte monitoring systems of the fourteenth and fifteenth arrangements may further comprise an overpatch. The overpatch may be configured in accordance with the overpatches described herein.

In the fourteenth and fifteenth arrangements, the on-body device and the sensor may be in accordance with those of the thirteenth arrangement.

In a sixteenth arrangement, of the on-body device, the housing may comprise a first part and a second part attached together; the electronics may comprise: processing electronics present on a semiconductor chip; and wireless communications electronics present on the semiconductor chip and configured to communicate according to a Bluetooth Low Energy protocol; a sensor connector; the sensor may have a flexible substrate, the sensor coupled with the sensor connector and comprising: a first portion retained within the housing and comprising three contacts configured to be in electrical contact with the electronics; and a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion, wherein the housing may comprise a recess configured to receive the sensor connector, and wherein the housing may be configured to be water-resistant when the sensor connector is received within the recess.

The on-body device may be provided such that: the sensor electronics further comprise a coin cell battery, a magnetic switch, a printed circuit board, and an antenna, wherein the antenna is configured to communicate according to the Bluetooth Low Energy protocol; the housing comprises a recess adapted to interface with an inserter projection; and the adhesive patch protrudes beyond a peripheral boundary of the housing; the sensor comprises a fold; and the adhesive patch is attached to the first part of the housing.

In a seventeenth arrangement, an analyte monitoring system is provided, comprising: an on-body device comprising a sensor; and an inserter configured to automatically advance a sharp, move the on-body device, and automatically retract the sharp, the inserter comprising: a removable cap; the sharp; a first spring configured to advance the sharp; a second spring configured to retract the sharp; a housing comprising an actuator configured to initiate advancement of the sharp. The sharp may comprise: a distal tip portion with a V-shaped portion having a first angle of 20 to 60 degrees; a proximal shaft portion comprising a first sidewall, a floor, and a second sidewall together providing a U-shape or a C-shape cross-sectional profile; a transition portion between the distal tip portion and the proximal shaft portion where the first sidewall and the second sidewall each have a height from the floor that is sloped at a second angle of 7 to 35 degrees. The first angle may be between 20 and 30 degrees and the second angle is between 8 and 18 degrees. The analyte monitoring system may further comprise: a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

In an eighteenth arrangement, an analyte monitoring system is provided, comprising: an on-body device comprising a sensor; and a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

The analyte monitoring systems of the seventeenth and eighteenth arrangements may further comprise an overpatch. The overpatch may be configured in accordance with the overpatches described herein.

In the seventeenth and eighteenth arrangements, the on-body device and the sensor may be in accordance with those of the sixteenth arrangement.

In a nineteenth arrangement of the on-body device, the housing may comprise a first part and a second part attached together; the electronics may comprise: processing electronics present on a semiconductor chip; and wireless communications electronics configured to communicate according to a Near Field Communication protocol and a Bluetooth Low Energy protocol, wherein at least a portion of the wireless communications electronics configured to communicate according to the Bluetooth Low Energy protocol are present on the semiconductor chip; the sensor may have a flexible substrate, and the sensor may be coupled with the sensor connector and comprise: a first portion retained within the housing and comprising three contacts configured to be in electrical contact with the sensor electronics; and a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion, wherein the housing may be configured to be water-resistant.

The on-body device may be provided such that: the sensor electronics further comprise a coin cell battery, a magnetic switch, a printed circuit board, a first antenna, and a second antenna; wherein the first antenna is configured to communicate according to the Bluetooth Low Energy protocol and the second antenna is configured to communicate according to the Near Field Communication protocol; the housing comprises a recess adapted to interface with an inserter projection; and the adhesive patch protrudes beyond a peripheral boundary of the housing; and the adhesive patch is attached to the first part of the housing. The coin cell battery may be electrically connected to the printed circuit board and is held adjacent to the printed circuit board such that the coin cell battery and the printed circuit board are both in a same plane.

In a twentieth arrangement, an analyte monitoring system is provided, comprising: an on-body device comprising a sensor; and an inserter configured to automatically advance a sharp, move the on-body device, and automatically retract the sharp, the inserter comprising: a removable cap; the sharp; a first spring configured to advance the sharp; a second spring configured to retract the sharp; a housing comprising an actuator configured to initiate advancement of the sharp. The on-body device may be housed within an interior space of the inserter, the interior space being sealed by the removable cap coupled with the housing of the inserter, and wherein the sharp and the sensor are maintained in a sterile environment by a sheath of the removable cap. The sharp may comprise: a distal tip portion with a V-shaped portion having a first angle of 20 to 60 degrees; the sharp comprises a proximal shaft portion comprising a first sidewall, a floor, and a second sidewall together providing a U-shape or a C-shape cross-sectional profile; the sharp comprises a transition portion between the distal tip portion and the proximal shaft portion where the first sidewall and the second sidewall each have a height from the floor that is sloped at a second angle of 7 to 35 degrees. The first angle may be between 27 and 37 degrees and the second angle is between 7 and 17 degrees. Th analyte monitoring system may further comprise: a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

In a twenty-first arrangement, an analyte monitoring system is provided, comprising: an on-body device comprising a sensor; and a receiver unit comprising a software application for execution on the receiver unit and a display, wherein the receiver unit is configured to receive analyte data associated with the analyte level of the user from the on-body device and to display information corresponding to the analyte data to the user, wherein the software application is configured to generate a present analyte value of the user, based on a measurement by the sensor, for output to the user not before expiration of a warm-up period of the sensor, and wherein the receiver unit and/or the on-body device are configured to apply a factory calibration to, in part, calculate an analyte value for display to the user.

The analyte monitoring systems of the twentieth and twenty-first arrangements may further comprise an overpatch. The overpatch may be configured in accordance with the overpatches described herein.

In the twentieth and twenty-first arrangements, the on-body device and the sensor may be in accordance with those of the nineteenth arrangement.

The on-body device of any of the first to twenty-first arrangements may have a designed wear duration that comprises 14 days.

The on-body device of any of the first to twenty-first arrangements may have a designed wear duration of 15 to 30 days.

The on-body device of any of the first to twenty-first arrangements may have a designed wear duration of 31 to 45 days.

The on-body device of any of the first to twenty-first arrangements may have a designed wear duration that is 45 days or greater.

The on-body device of any of the first to twenty-first arrangements may have a designed wear duration of 14 days.

The on-body device of any of the first to twenty-first arrangements may have a designed wear duration of 15 days.

The on-body device of any of the first to twenty-first arrangements may have a designed wear duration of 21 days.

The on-body device of any of the first to twenty-first arrangements may have a designed wear duration of 28 days.

Aspects of the present disclosure may be provided in conjunction with each other and features of one aspect may be applied to other aspects.

### BRIEF DESCRIPTION OF THE FIGURES

Embodiments of the present disclosure are described below, by way of example only, with reference to the accompanying figures. It is to be understood that this disclosure is not limited to the particular embodiments described, and instead the scope of the invention is defined by the appended claims.

Unless defined otherwise, all technical and scientific terms used herein have the same ordinary meaning as commonly understood by one of ordinary skill in the art to which the present disclosure belongs.

The figures shown are not necessarily drawn to scale, with some components and features exaggerated or omitted for clarity.

FIG. 1A shows a block diagram of an analyte monitoring system according to an embodiment.

FIG. 1B-1 shows a cross-sectional view of an on-body device according to an embodiment.

FIG. 1B-2 shows a perspective view of the on-body device of FIG. 1B-1.

FIG. 1B-3 shows a bottom view of the on-body device of FIG. 1B-1, with the adhesive patch removed.

FIG. 1B-4 shows a cross-sectional view of an on-body device according to an embodiment.

FIG. 1C-1 shows a perspective view of an on-body device according to an embodiment, having a square shape.

FIG. 1C-2 shows a bottom view of the on-body device of FIG. 1C-1, with the adhesive patch removed.

FIG. 1D shows a cross-sectional view of an on-body device according to an embodiment, having an angled side surface.

FIG. 1E shows a cross-sectional view of an on-body device according to an embodiment, having a curved side surface.

FIG. 1F shows a cross-sectional view of an on-body device according to an embodiment, without a side surface.

FIG. 1G shows a cross-sectional view of an on-body device according to an embodiment, having an intermediate surface which is angled.

FIG. 1H shows a cross-sectional view of an on-body device according to an embodiment, having an intermediate surface which is curved.

FIG. 1I shows an exploded cross-sectional view of an on-body device according to an embodiment, having two parts for assembly.

FIG. 1J-1 shows an exploded cross-sectional view of an on-body device according to an embodiment, having a sensor connector for assembly through the top surface.

FIG. 1J-2 shows a cross-sectional view of the on-body device of FIG. 1J-1, with the sensor connector assembled.

FIG. 1K-1 shows an exploded cross-sectional view of an on-body device according to an embodiment, having a sensor connector for assembly through the base surface.

FIG. 1K-2 shows a cross-sectional view of the on-body device of FIG. 1K-1, with the sensor connector assembled.

FIG. 1L-1 shows an exploded cross-sectional view of an on-body device according to an embodiment, having an analyte sensor and filling material for assembly through the base surface.

FIG. 1L-2 shows a cross-sectional view of the on-body device of FIG. 1L-1, with the analyte sensor and filling material assembled.

FIG. 1M-1 shows an exploded cross-sectional view of an on-body device according to an embodiment, having sensor electronics for assembly after sensor insertion.

FIG. 1M-2 shows a cross-sectional view of the on-body device of FIG. 1M-1, with the electronics assembled.

FIG. 1N-1 shows an exploded top perspective view of an on-body device according to an embodiment with the analyte sensor of FIG. 1Q-1.

FIG. 1N-2 shows an exploded bottom perspective view of the on-body device of the embodiment of FIG. 1N-1.

FIGS. 1N-3 to 1N-20 show bottom views of on-body devices with different housing shapes according to embodiments.

FIGS. 1O-1 and 1O-2 show perspective views of partially implantable analyte sensors according to embodiments.

FIG. 1O-3 shows a cross-sectional view of a portion of a partially implantable analyte sensor according to an embodiment.

FIG. 1O-4 shows a side view of a partially implantable analyte sensor according to an embodiment.

FIG. 1O-5 shows an end on view of a partially implantable analyte sensor according to an embodiment.

FIGS. 1P-1 and 1P-2 show perspective views of partially implantable analyte sensors according to embodiments.

FIG. 1P-3 shows a perspective view of a partially implantable analyte sensor according to an embodiment.

FIG. 1Q-1 shows a perspective view of an analyte sensor according to an embodiment.

FIG. 1Q-2 shows a perspective view of an analyte sensor according to an embodiment.

FIGS. 1R-1 and 1R-2 show a block diagrams of sensor electronics according to embodiments.

FIGS. 1R-3 and 1R-4 show top down views of a printed circuit board according to embodiments.

FIG. 1R-5 shows a bottom up view of a printed circuit board according to an embodiment.

FIG. 1R-6 shows a side view of a printed circuit board according to an embodiment.

FIG. 1R-7 shows a top down view of a printed circuit board according to an embodiment.

FIG. 1R-8 shows a bottom up view of a printed circuit board according to an embodiment.

FIG. 1R-9 shows a side view of a printed circuit board according to an embodiment.

FIG. 1R-10 shows a bottom up view of a housing of an on-body device according to an embodiment.

FIG. 1R-11 shows a cross-sectional side view of an on-body device according to an embodiment.

FIG. 1R-12 shows a cross-sectional side view of an on-body device according to an embodiment.

FIG. 1R-13 shows a block diagram of receiver electronics according to an embodiment.

FIG. 1S-1 shows a cross-sectional view of an inserter according to an embodiment, before insertion.

FIG. 1S-2 shows a cross-sectional view of the inserter of FIG. 1S-1, after insertion.

FIGS. 1T-1 to 1T-3 show perspective views of different stages of an insertion procedure according to an embodiment.

FIGS. 1T-4 to 1T-6 show perspective views of different stages of an insertion procedure according to an embodiment.

FIGS. 1T-7 to 1T-9 show perspective views of different stages of an insertion procedure according to an embodiment.

FIGS. 1T-10 and 1T-11 show perspective views of different stages of an application procedure according to an embodiment.

FIGS. 1T-12 and 1T-13 show perspective views of different stages of an application procedure according to an embodiment.

FIG. 1U-1 shows a cross-sectional view of an inserter according to an embodiment, before insertion.

FIG. 1U-2 shows a cross-sectional view of the inserter of FIG. 1U-1, during insertion.

FIG. 1U-3 shows a cross-sectional view of the inserter of FIG. 1U-1, during insertion.

FIG. 1U-4 shows a cross-sectional view of an inserter according to an embodiment, before insertion.

FIG. 1U-5 shows a cross-sectional view of the inserter of FIG. 1U-4, during insertion.

FIG. 1U-6 shows a cross-sectional view of the inserter of FIG. 1U-4, during insertion.

FIG. 1U-7 shows a cross-sectional view of an inserter and a container according to an embodiment, before loading.

FIG. 1U-8 shows a cross-sectional view of the inserter and container of FIG. 1U-7, after loading.

FIG. 1U-9 shows a cross-sectional view of an inserter and a container according to an embodiment, before loading.

FIG. 1U-10 shows a cross-sectional view of the inserter and container of FIG. 1U-9, after loading.

FIG. 1U-11 shows a cross-sectional view of an inserter and a container according to an embodiment, before loading.

FIG. 1U-12 shows a cross-sectional view of the inserter and container of FIG. 1U-11, after loading.

FIG. 1U-13 shows a perspective view of an inserter according to an embodiment.

FIG. 1U-14 shows a cross-sectional view of an inserter according to an embodiment, before end cap removal.

FIG. 1U-15 shows a cross-sectional view of the inserter of FIG. 1U-14 after end cap removal.

FIG. 1U-16 shows a perspective view of a device support and an on-body device according to an embodiment.

FIG. 1U-17 shows a partial cross-sectional view of the device support and on-body device of FIG. 1U-16 when coupled together.

FIG. 1U-18 shows a perspective view of a device support and an on-body device according to an embodiment.

FIG. 1U-19 shows a partial cross-sectional view of the device support and on-body device of FIG. 1U-18 when coupled together.

FIG. 1U-20 shows a top down view of an on-body device according to an embodiment.

FIG. 1U-21 shows a perspective view of a device support and an on-body device according to an embodiment.

FIG. 1U-22 shows a partial cross-sectional view of the device support and the on-body device of FIG. 1U-21 when coupled together.

FIG. 1U-23 shows a side view of a sharp according to an embodiment.

FIG. 1U-24 shows an end on view of a sharp according to an embodiment.

FIG. 1U-25 shows an end on view of a sharp according to an embodiment.

FIG. 1U-26 shows an end on view of a sharp according to an embodiment.

FIG. 1U-27 shows an end on view of a sharp according to an embodiment.

FIG. 1U-28 shows an end on view of a sharp according to an embodiment.

FIG. 1U-29 shows a top down view of a sharp according to an embodiment.

FIG. 1U-30 shows a side view of the sharp of FIG. 1U-29.

FIG. 2A-1 shows a cross-sectional view of an on-body device having a vent according to an embodiment.

FIG. 2A-2 shows a perspective view of the on-body device of FIG. 2A-1.

FIG. 2A-3 shows a bottom view of the on-body device of FIG. 2A-1, with an adhesive patch removed.

FIG. 2A-4 shows a bottom view of the on-body device of FIG. 2A-1, with the adhesive patch present.

FIGS. 2B-1 and 2B-2 show cross-sectional views of on-body devices having angled vent passageways according to embodiments.

FIGS. 2C-1 and 2C-2 show cross-sectional views of on-body devices having a vent passageway with a tapered configuration according to embodiments.

FIG. 2D shows a cross-sectional view of an on-body device having a vent passageway with a stepped configuration according to an embodiment.

FIGS. 2E-1 and 2E-2 show cross-sectional views of an on-body device having a vent passageway connected to a side surface according to embodiments.

FIGS. 2F-1 and 2F-2 show cross-sectional views of an on-body device having a vent passageway connected to a sensor passageway according to embodiments.

FIG. 2G shows a cross-sectional view of an on-body device having a plurality of vent passageways according to an embodiment.

FIG. 2H-1 shows an exploded perspective view from above of an on-body device having a plurality of vent passageways according to an embodiment.

FIG. 2H-2 shows an exploded perspective view from below the on-body device of FIG. 2H-1.

FIG. 2I-1 shows a cross-sectional view of an on-body device having a vent compartment according to an embodiment.

FIG. 2I-2 shows a perspective view of the on-body device of FIG. 2I-1.

FIG. 2I-3 shows a bottom view of the on-body device of FIG. 2I-1, with an adhesive patch removed.

FIG. 2I-4 shows a cross-sectional view of an on-body device having a vent compartment according to an embodiment.

FIG. 2I-5 shows a cross-sectional view of an on-body device having a vent compartment according to an embodiment.

FIG. 2J-1 shows a cross-sectional view of an on-body device having a vent passageway connected to channels in a base surface according to an embodiment.

FIG. 2J-2 shows a bottom view of the on-body device of FIG. 2J-1, with an adhesive patch removed.

FIGS. 2K-1 to 2K-10 show perspective views of an on-body device having vent passageways according to embodiments.

FIGS. 2K-11 to 2K-12 show perspective views of an on-body device having vent passageways according to embodiments.

FIGS. 3A-1 and 3A-2 show cross-sectional views of on-body devices having passageways with moisture permeable seals according to embodiments.

FIGS. 3B-1, 3B-2, 3B-3, and 3B-4 show cross-sectional views of on-body devices having passageways with moisture permeable seals according to embodiments.

FIGS. 3C-1, 3C-2, 3C-3, and 3C-4 show cross-sectional views of on-body devices having passageways with moisture permeable seals according to embodiments.

FIGS. 3D-1 and 3D-2 show cross-sectional views of on-body devices having passageways with moisture permeable seals according to embodiments.

FIGS. 3E-1 and 3E-2 show cross-sectional views of on-body devices having passageways with moisture permeable seals according to embodiments.

FIGS. 3E-3 and 3E-4 show cross-sectional views of on-body devices having passageways with moisture permeable seals according to embodiments.

FIGS. 3F-1 and 3F-2 show cross-sectional views of on-body devices having passageways with moisture permeable seals according to embodiments.

FIGS. 3F-3 and 3F-4 show cross-sectional views of on-body devices having passageways with moisture permeable seals according to embodiments.

FIGS. 3G-1 and 3G-2 show cross-sectional views of on-body devices having passageways with moisture permeable seals according to embodiments.

FIGS. 3H-1 and 3H-2 show cross-sectional views of an on-body device having passageway in the form of a vent compartment with a moisture permeable seal according to embodiments.

FIGS. 3I-1 and 3I-2 show cross-sectional views of on-body devices having base channels and passageways, where the passageways have moisture permeable seals according to embodiments.

FIGS. 4A-1 and 4A-2 show cross-sectional views of a passageway with recesses in an on-body device housing before and after placement of a moisture permeable seal according to an embodiment.

FIGS. 4B-1 and 4B-2 show cross-sectional views of a passageway with projections in an on-body device housing before and after placement of a moisture permeable seal according to an embodiment.

FIGS. 5A-1, 5A-2 and 5A-3 show cross-sectional views of a passageway with a recess in an on-body device housing before, during and after placement of a moisture permeable seal according to an embodiment.

FIG. 5A-4 shows a perspective view of the surface of the passageway of the embodiment of FIGS. 5A-1, 5A-2, and 5A-3.

FIG. 5A-5 shows a perspective view of the moisture permeable seal of the embodiment of FIGS. 5A-1, 5A-2, and 5A-3.

FIG. 5B shows a cross-sectional view of a moisture permeable seal in an upper location of a sensor passageway of an on-body device housing according to an embodiment.

FIG. 5C-1 shows a cross-sectional view of a moisture permeable seal in a lower location of a sensor passageway of an on-body device housing according to an embodiment.

FIG. 5C-2 shows a cross-sectional view of the sensor, moisture permeable seal, and passageway taken along line 5C2-5C2 of FIG. 5C-1.

FIGS. 5D-1 and 5D-2 show cross-sectional views of a passageway with a projection in an on-body device housing before and after placement of a moisture permeable seal according to an embodiment.

FIG. 5D-3 shows a perspective view of the moisture permeable seal of the embodiment of FIGS. 5D-1 and 5D-2.

FIGS. 5E-1 and 5E-2 show cross-sectional views of a passageway in an on-body device housing before and after placement of a moisture permeable seal according to an embodiment.

FIG. 5E-3 shows a perspective view of the moisture permeable seal of the embodiment of FIGS. 5E-1 and 5E-2.

FIGS. 6A-1 and 6A-2 show perspective views of an on-body device before and after placement of a moisture permeable seal according to an embodiment.

FIG. 6A-3 shows a side view of the on-body device having the moisture permeable seal of the embodiment of FIG. 6A-2.

FIG. 6B-1 shows a perspective view of an on-body device before placement of a moisture permeable seal over a sharp aperture according to an embodiment.

FIG. 6B-2 shows a cross-sectional view of a sensor passageway in an on-body device housing before placement of a moisture permeable seal taken along line 6B2-6B2 of the embodiment of FIG. 6B-1.

FIG. 6B-3 shows a cross-sectional view of a sensor passageway in an on-body device housing after placement of a moisture permeable seal taken along line 6B2-6B2 of the embodiment of FIG. 6B-1.

FIGS. 7A-1 through 7A-6 show cross-sectional views of foam seals within a passageway according to embodiments.

FIGS. 7B-1 through 7B-8 show cross-sectional views of foam seals in combination with moisture permeable seals within a passageway according to embodiments.

FIG. 8A-1 shows a bottom view of an on-body device with channels and with the adhesive patch removed, according to an embodiment.

FIG. 8A-2 shows a perspective view of the on-body device of FIG. 8A-1.

FIG. 8A-3 shows another perspective view of the on-body device of FIG. 8A-1.

FIG. 8A-4 shows a cross-sectional view of the on-body device of FIG. 8A-1.

FIG. 8A-5 shows a perspective, cross-sectional view of the on-body device of FIG. 8A-1, having U-shaped channels, with a magnification thereof.

FIG. 8A-6 shows a perspective, cross-sectional view of the on-body device of FIG. 8A-1, having V-shaped channels, with a magnification thereof.

FIG. 8A-7 shows a perspective, cross-sectional view of the on-body device of FIG. 8A-1, having three-sided (rectangular) shaped channels, with a magnification thereof.

FIG. 8A-8 shows a perspective, cross-sectional view of the on-body device of FIG. 8A-1, having channels with a fillet edge, with a magnification thereof.

FIG. 8A-9 shows a perspective, cross-sectional view of the on-body device of FIG. 8A-1, having channels with a chamfer edge, with a magnification thereof.

FIG. 8A-10 shows a perspective view of the on-body device of FIG. 8A-1, having moisture permeable material on proximal portions of the base.

FIG. 8A-11 shows a perspective view of the on-body device of FIG. 8A-1, having moisture permeable material within channels.

FIG. 8A-12 shows a perspective view of the on-body device of FIG. 8A-1, having moisture permeable material on proximal portions of the base and within channels.

FIG. 8B-1 shows a perspective view of an on-body device according to an embodiment, having channels with variable depth, with a magnification thereof.

FIG. 8B-2 shows a perspective view of an on-body device according to an embodiment, having channels with variable depth, with a magnification thereof.

FIG. 8C shows a perspective view of an on-body device according to an embodiment, having channels with variable width.

FIG. 8D-1 shows a bottom view of an on-body device according to an embodiment, having channels with non-linearly variable depth and width, with magnifications thereof.

FIG. 8D-2 shows a perspective view of the on-body device of FIG. 8D-1, with magnifications thereof.

FIG. 8E shows a chart indicating the relationship between moisture loss rates and the measured depth of the channels.

FIG. 9A shows a perspective view of a base of an on-body device having vents and channels, according to an embodiment.

FIG. 9B shows a perspective view of a base of an on-body device having vents and channels, according to an embodiment.

FIG. 9C shows a perspective view of a base of an on-body device having vents and channels, according to an embodiment.

FIG. 9D shows a perspective view of a base of an on-body device having vents and channels, according to an embodiment.

FIG. 10 shows a bottom view of an on-body device with a triangular shape and with channels, according to an embodiment.

FIGS. 11A to 11C show bottom views and zoomed portions of on-body devices with directional surface texture, according to embodiments.

FIG. 11D shows a bottom view of an on-body device with directional surface texture in a circular pattern, according to embodiments.

FIG. 11E shows a bottom view of an on-body device with directional surface texture in a polygonal pattern, according to embodiments.

FIG. 11F shows a bottom view of an on-body device with directional surface texture in a radial pattern, according to embodiments.

FIGS. 12A to 12E show bottom views of on-body devices with channels and directional surface texture, according to embodiments.

FIG. 12F shows a cross-sectional view of the on-body device of FIG. 12D along line A-A, with directional surface texture applied to the proximal portions.

FIG. 12G shows a cross-sectional view of the on-body device of FIG. 12E along line B-B, with directional surface texture applied to the proximal portions and within the channels.

FIGS. 13A-1, 13B-1 and 13C-1 show bottom views of on-body devices with non-directional surface texture, according to embodiments.

FIGS. 13A-2, 13B-2 and 13C-2 show zoomed in cross-sectional views of the non-directional surface texture on the on-body devices of FIGS. 13A-1, 13B-1 and 13C-1 respectively.

FIGS. 14A-1 and 14B-1 show bottom views of on-body devices with channels and non-directional surface texture, according to embodiments.

FIGS. 14A-2 and 14B-2 show zoomed in cross-sectional views of the non-directional surface texture on the on-body devices of FIGS. 14A-1 and 14B-1 respectively.

FIG. 15-1 provides a plan view of an adhesive patch assembly according to an embodiment of the disclosure.

FIG. 15-2 provides a side view of an adhesive patch assembly according to the embodiment of the disclosure shown in FIG. 15-1.

FIG. 16 provides a plan view of an adhesive patch assembly according to another embodiment of the disclosure.

FIG. 17 provides a plan view of an adhesive patch assembly according to an embodiment of the disclosure.

FIG. 18 provides a plan view of an adhesive patch assembly according to another embodiment of the disclosure.

FIG. 19 provides a plan view of an adhesive patch assembly according to another embodiment of the disclosure.

FIG. 20 provides a plan view of an adhesive patch assembly according to another embodiment of the disclosure.

FIG. 21 provides a plan view of an adhesive patch assembly according to another embodiment of the disclosure.

FIG. 22 provides a plan view of an adhesive patch assembly according to another embodiment of the disclosure.

FIG. 23 provides a plan view of an adhesive patch assembly according to another embodiment of the disclosure.

FIG. 24 provides a plan view of an adhesive patch assembly according to another embodiment of the disclosure, wherein the adhesive patch assembly makes use of a path-following removable patch portion.

FIG. 25 provides a plan view of an adhesive patch assembly according to another embodiment of the disclosure which makes use of a path-following removable patch portion.

FIG. 26 provides a plan view of an adhesive patch assembly according to another embodiment of the disclosure which makes use of a path-following removable patch portion.

FIG. 27-1 provides a plan view of an adhesive patch assembly according to another embodiment of the disclosure, wherein the adhesive patch assembly comprises two layers of backing material.

FIG. 27-2 provides a plan view of an adhesive patch assembly according to another embodiment of the disclosure, wherein the adhesive patch assembly comprises three layers of backing material.

FIG. 27-3 provides a plan view of an adhesive patch assembly according to another embodiment of the disclosure, wherein the adhesive patch assembly comprises two layers of backing material.

FIG. 28 provides a plan view of an adhesive patch assembly illustrating various techniques for assisting in the initiation of removal of a removal patch portion.

FIG. 29 provides an illustration of how a releasable coupling may extend to a given edge.

FIG. 30 provides an illustration as to how the external corner of a removable patch portion may be adjusted to minimize its chance of unintentional peeling.

FIG. 31 shows a perspective view of a reinforcement device comprising reinforcement legs that are coupled to the housing of an OBD according to an embodiment.

FIG. 32A shows a perspective view of a reinforcement device comprising reinforcement legs that are coupled to a body portion that is coupled to a housing of an OBD according to an embodiment.

FIG. 32B shows a perspective view of a reinforcement device comprising reinforcement legs that are coupled to a body portion that is coupled to a housing of an OBD according to an embodiment.

FIG. 33A shows a perspective view of a reinforcement device comprising a plate shaped body portion applied to a housing of an OBD according to an embodiment.

FIG. 33B shows a perspective view of a reinforcement device similar to the reinforcement device of FIG. 33A and further comprising a connection member that couples adjacent reinforcement legs according to an embodiment.

FIG. 34 shows a perspective view of a reinforcement device comprising arcuate legs applied to a housing of an OBD according to an embodiment.

FIG. 35 shows a perspective view of reinforcement device comprising a single reinforcement band applied to the housing of an OBD according to an embodiment.

FIG. 36A shows a perspective view of a reinforcement device comprising a plurality of reinforcement bands applied to an OBD according to an embodiment.

FIG. 36B shows a perspective view of a reinforcement device comprising a plurality of reinforcement bands applied to an OBD according to another embodiment.

FIG. 36C shows a perspective view of a reinforcement device similar to that of FIG. 36A but additionally comprising webbing and a cover band applied to an OBD according to an embodiment.

FIG. 37 shows a perspective view of a reinforcement device comprising a reinforcement band with arcuate channels applied to an OBD according to an embodiment.

FIG. 38A shows a perspective view of a reinforcement device comprising a plurality of discrete beads applied to an OBD according to an embodiment.

FIG. 38B shows a magnified view of the reinforcement device of FIG. 38A.

FIG. 39A shows a top view of a reinforcement device comprising a plurality of concentric and co-planar reinforcement bands applied to an OBD according to an embodiment.

FIG. 39B shows a cross-sectional view of the reinforcement device of FIG. 39A.

FIG. 40A shows a top view of a reinforcement device comprising a plurality of concentric stacked reinforcement bands applied to an OBD according to an embodiment.

FIG. 40B shows a cross-sectional view of the reinforcement device of FIG. 40A.

FIG. 41A shows a side view of a non-isotropic patch according to an example embodiment.

FIG. 41B shows a top down view of an upper surface of a non-isotropic layer of a patch according to an example embodiment.

FIG. 42A shows a top down view of a non-isotropic patch configured for use as an on-body device patch according to an example embodiment.

FIG. 42B shows a side view of an embodiment of an on-body device having a non-isotropic patch adhered according to an example embodiment.

FIG. 42C shows a top down view of a non-isotropic patch configured for use as an on-body device patch according to another example embodiment.

FIG. 42D shows a side view of an embodiment of an on-body device having a non-isotropic patch adhered according to an example embodiment.

FIG. 42E shows a top down view of a non-isotropic patch configured for use as an overpatch according to another example embodiment.

FIG. 42G shows a top down view of a non-isotropic patch configured for use as an overpatch according to another example embodiment.

FIG. 42H shows a side view of this embodiment of non-isotropic patch adhered over an embodiment of an on-body device.

FIG. 42I-1 shows a top down view of an example embodiment of a non-isotropic patch according to an example embodiment.

FIG. 42I-2 shows a top down view of an example embodiment of an on-body device with a non-isotropic patch according to an example embodiment.

FIG. 42I-3 shows a top down view of an example embodiment of an on-body device with a non-isotropic patch according to an example embodiment.

FIG. 42J-1 shows a top down view of a non-isotropic patch according to an example embodiment.

FIG. 42J-2 shows a top down view of the non-isotropic patch of FIG. 42J-1 in an open state.

FIG. 42J-3 shows a top down view of a non-isotropic patch according to an example embodiment.

FIG. 42J-4 shows a top down view of the non-isotropic patch of FIG. 42J-3 in an open state.

FIG. 42K-1 shows a top down view of a non-isotropic patch according to an example embodiment.

FIG. 42K-2 shows a top down view of a non-isotropic patch according to an example embodiment.

FIG. 42K-3 shows a top down view of a non-isotropic patch according to an example embodiment.

FIG. 42L shows a top down view of a non-isotropic patch according to an example embodiment.

FIG. 42M-1 shows a top down view of a non-isotropic patch according to an example embodiment.

FIG. 42M-2 shows a top down view of a non-isotropic patch according to an example embodiment.

FIG. 42M-3 shows a top down view of a non-isotropic patch according to an example embodiment.

FIG. 42N shows a top down view of a non-isotropic patch according to an example embodiment.

FIG. 42O shows a top down view of a non-isotropic patch according to an example embodiment.

FIG. 42P shows a side view of a non-isotropic patch according to an example embodiment.

FIG. 43A shows a side view of an overpatch assembly for an on-body device according to an example embodiment.

FIG. 43B shows an unassembled side view of the overpatch assembly of FIG. 43A.

FIG. 43C shows an elevated perspective view of exploded overpatch assembly of FIG. 43A.

FIG. 43D shows a bottom up view of the overpatch assembly of FIG. 43A in a fully assembled state.

FIG. 43E shows a bottom up view of an overpatch assembly device according to another example embodiment.

FIG. 43F shows a top down view of another example embodiment of an overpatch assembly.

FIG. 43G shows a bottom up view of the overpatch assembly of FIG. 43F.

FIG. 43H shows a side view of the overpatch assembly of FIG. 43F.

FIG. 43I-1 is an exploded view showing example embodiments of components of the overpatch assembly of FIG. 43F in their unassembled and unfolded states.

FIG. 43I-2 shows a bottom view of the components of FIG. 43I-1 of an overpatch assembly during application.

FIG. 43J-1 is an exploded view showing additional example embodiments of components of the overpatch assembly of FIG. 43F in their unassembled and unfolded states.

FIG. 43J-2 shows a bottom view of the components of FIG. 43J-1 of the overpatch assembly during application.

FIG. 43J-3 is an exploded view showing additional example embodiments of components of the overpatch assembly of FIG. 43F in their unassembled and unfolded states.

FIG. 43J-4 shows a side view of the overpatch assembly of FIGS. 43J-1 and 43J-2.

FIG. 43J-5 is an exploded view showing additional example embodiments of components of an overpatch assembly in their unassembled and unfolded states.

FIG. 43J-6 shows a bottom view of the components of FIG. 43J-5 of the overpatch assembly during application.

FIG. 43J-7 is an exploded view showing additional example embodiments of components of an overpatch assembly in their unassembled and unfolded states.

FIG. 43J-8 shows a bottom view of the components of FIG. 43J-7 of the overpatch assembly during application.

FIG. 43K-1 shows a bottom up view of a package in which an overpatch assembly can be provided to the user according to an example embodiment.

FIG. 43K-2 shows a bottom up view of a package in which an overpatch assembly can be provided to the user according to another example embodiment.

FIG. 44A shows a top down perspective view of an overpatch assembly according to an example embodiment.

FIG. 44B shows a bottom up perspective view of the overpatch assembly of FIG. 44A.

FIG. 44C shows an exploded view of the overpatch assembly of FIG. 44A.

FIG. 44D-1 shows a perspective view of a support structure according to an example embodiment.

FIG. 44D-2 shows a bottom up view of the support structure of FIG. 44D-1.

FIG. 44D-3 shows a perspective view of the support structure of FIG. 44D-1.

FIG. 44E shows a top down perspective view of a support structure according to another example embodiment.

FIG. 44F shows a top down perspective view of a support structure according to another example embodiment.

FIG. 44G-1 shows a top down view of an on-body device according to an example embodiment.

FIG. 44G-2 shows a bottom up view of a support structure according to an example embodiment.

FIG. 44H-1 shows a top down view of an on-body device according to an example embodiment.

FIG. 44H-2 shows a bottom up view of a support structure according to an example embodiment.

FIG. 44I-1 shows a top down perspective view of an overpatch assembly according to an example embodiment.

FIG. 44I-2 shows a bottom up perspective view of the overpatch assembly of FIG. 44I-1.

FIG. 44J shows a top down view of the overpatch assembly of FIG. 44A during application.

FIG. 44K shows a top down view of the overpatch assembly of FIG. 44A during application.

FIG. 44L shows a top down view of the overpatch assembly of FIG. 44A during application.

FIG. 45 shows a side view of a construction of an on-body device with a patch according to an example embodiment.

FIG. 46A shows a landscape view of an analyte monitoring system according to an embodiment.

FIG. 46B shows a landscape view of an analyte monitoring system according to an embodiment.

FIG. 46C shows a landscape view of an analyte monitoring system according to an embodiment.

FIG. 46D shows a landscape view of an analyte monitoring system according to an embodiment.

FIG. 46E shows a landscape view of an analyte monitoring system according to an embodiment.

FIG. 46F shows a landscape view of an analyte monitoring system according to an embodiment.

FIG. 46G shows a landscape view of an analyte monitoring system according to an embodiment.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

### Analyte Monitoring System

As used herein the term “analyte” refers to a target substance for detection according to a chemical technique, an electrical technique, an electro-chemical technique, a photonic technique based on non-ionizing radiation, or a combination of two or more thereof.

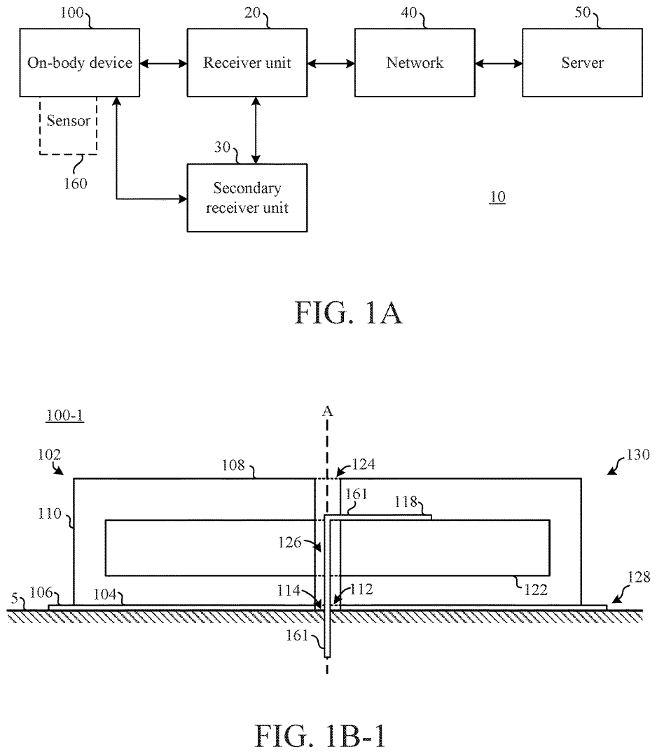

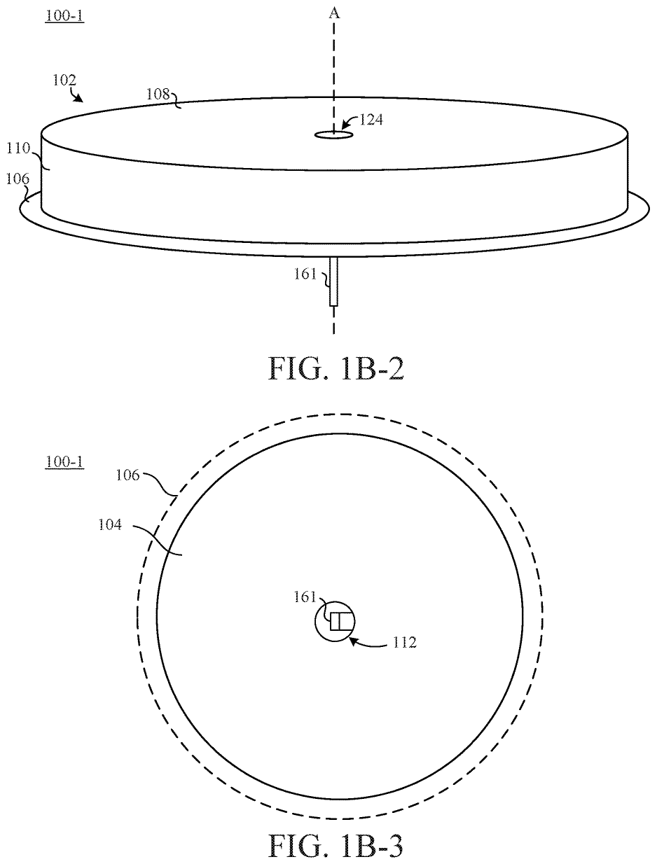

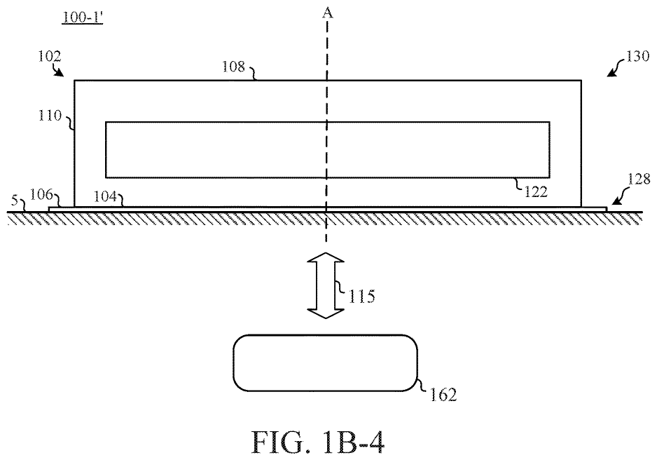

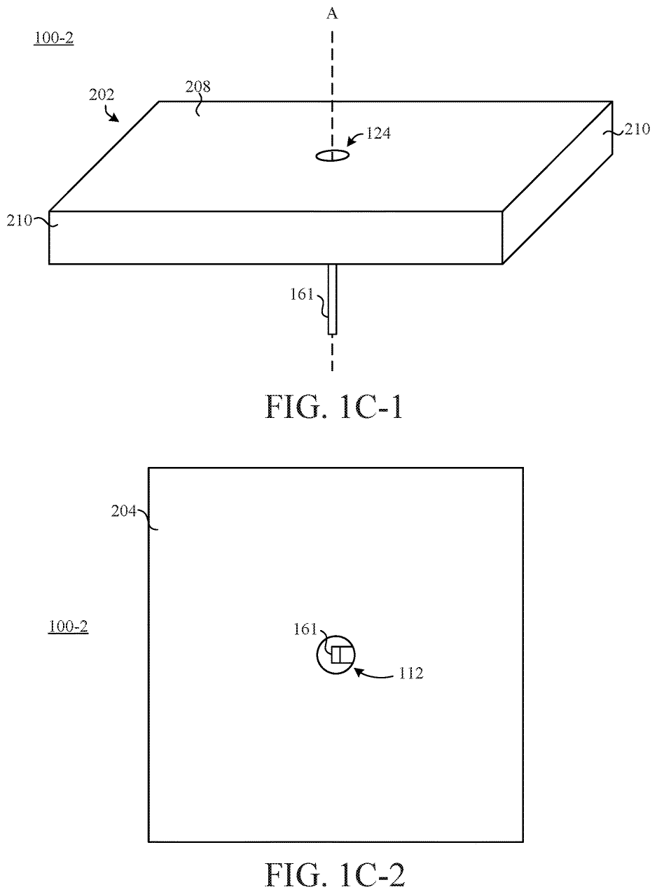

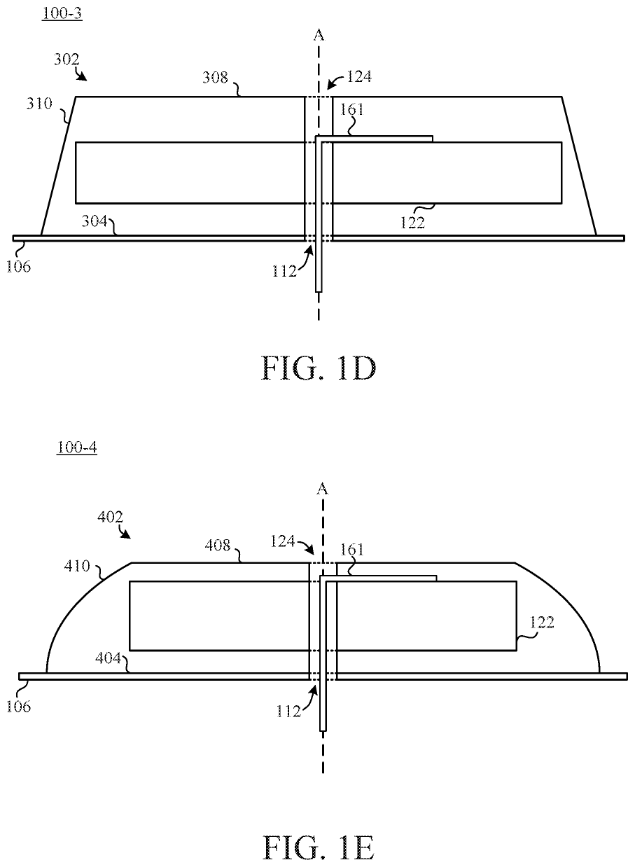

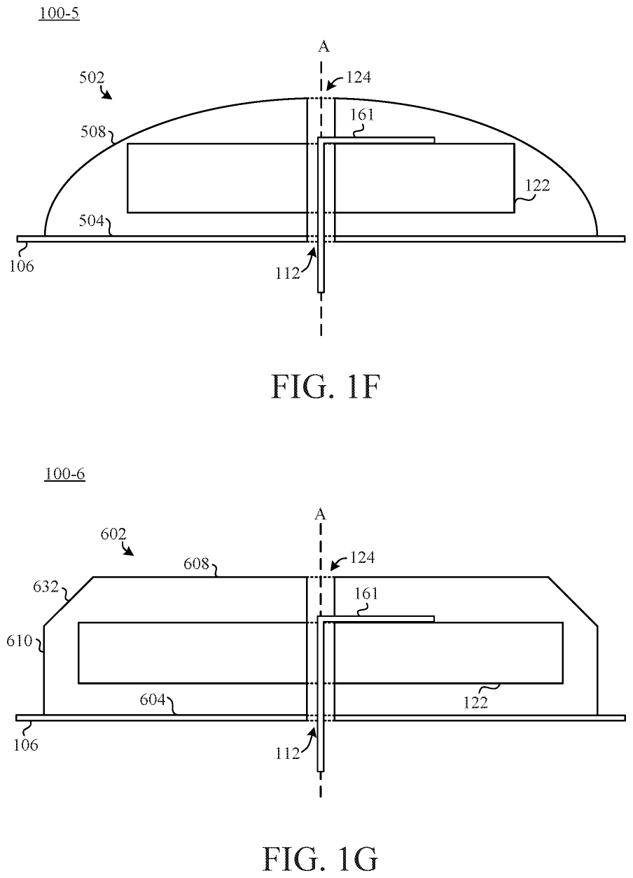

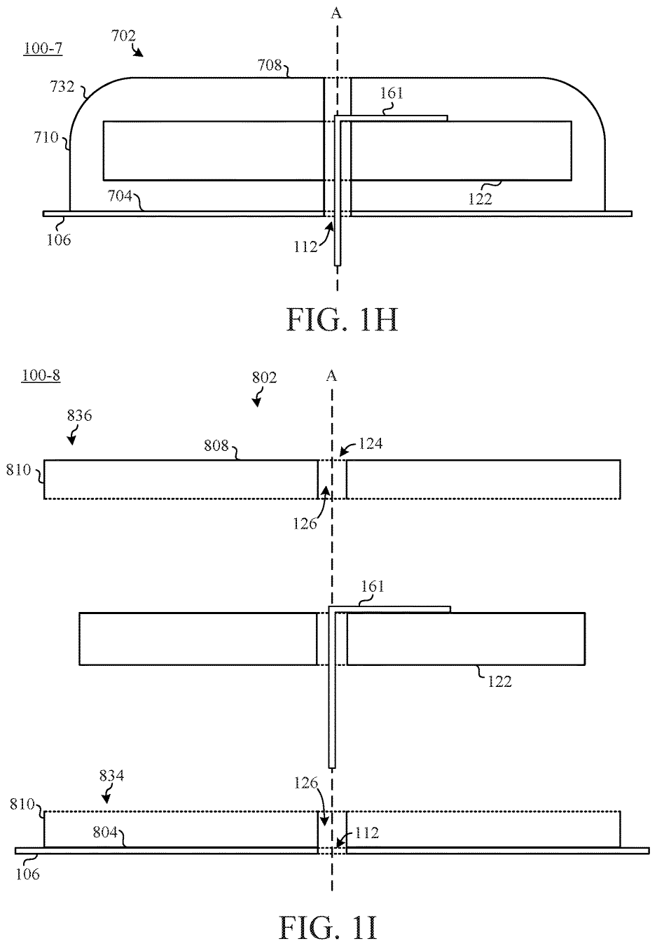

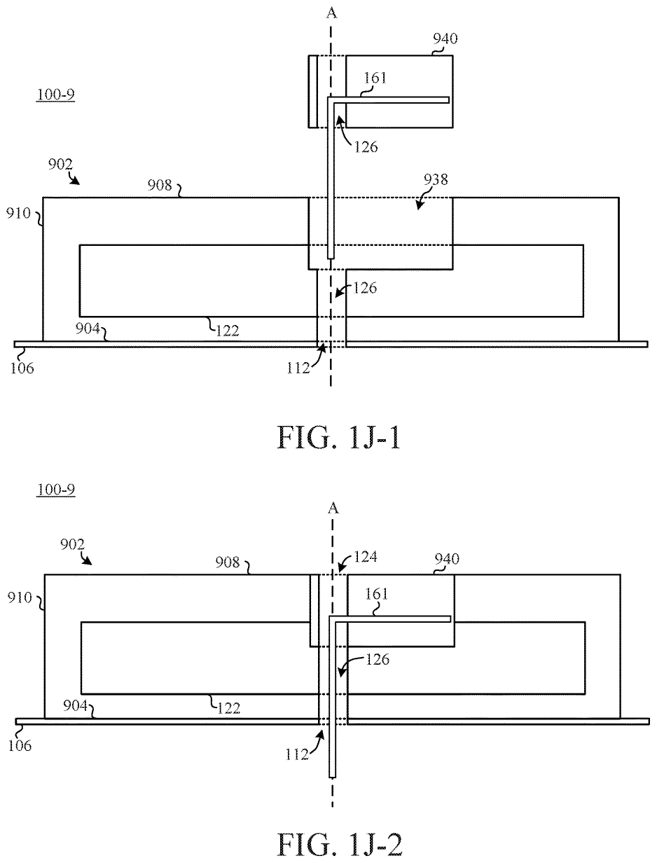

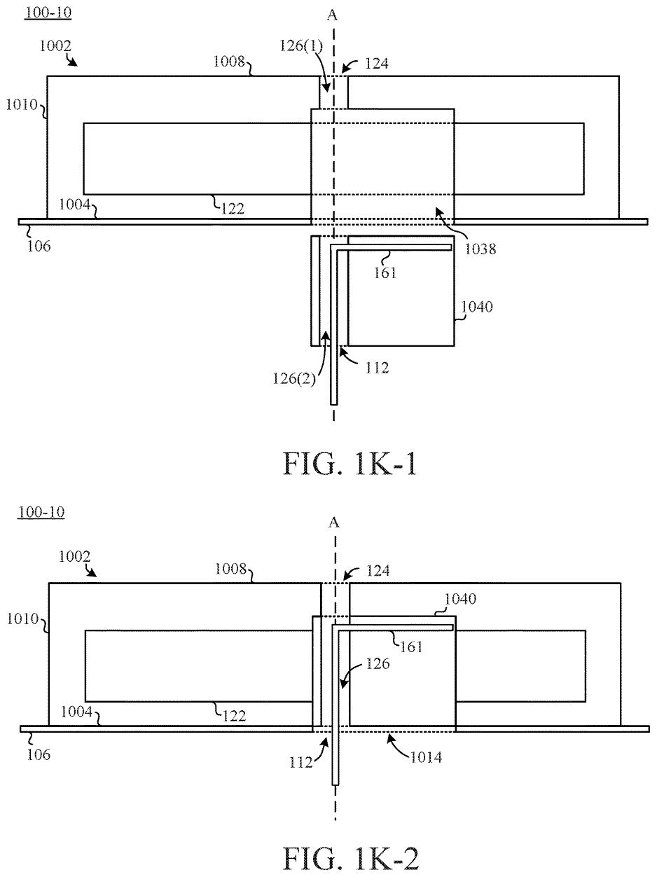

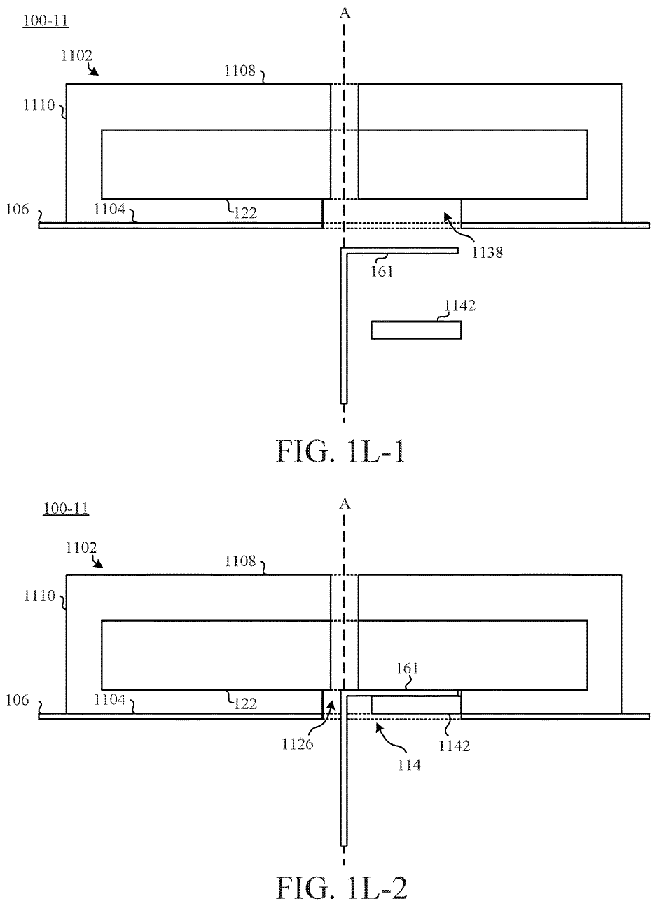

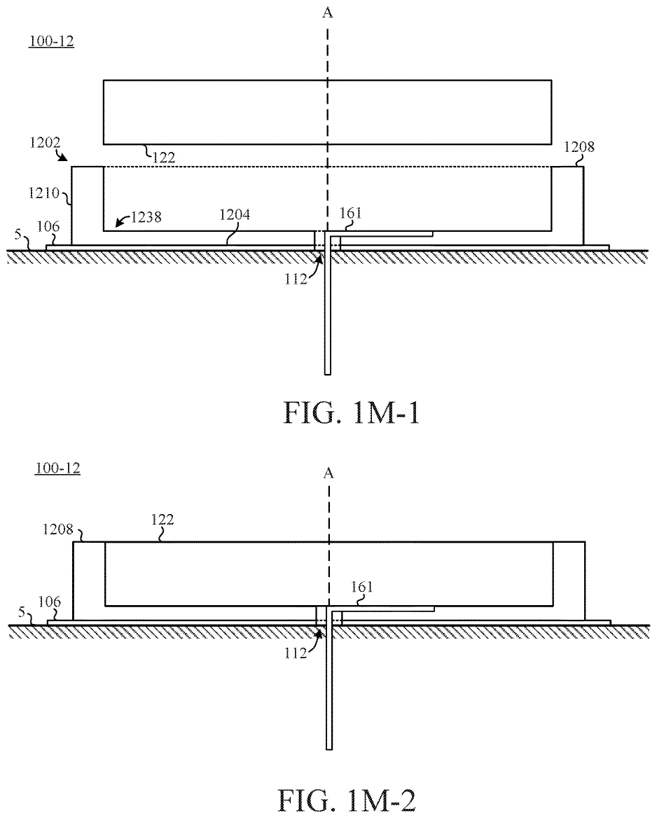

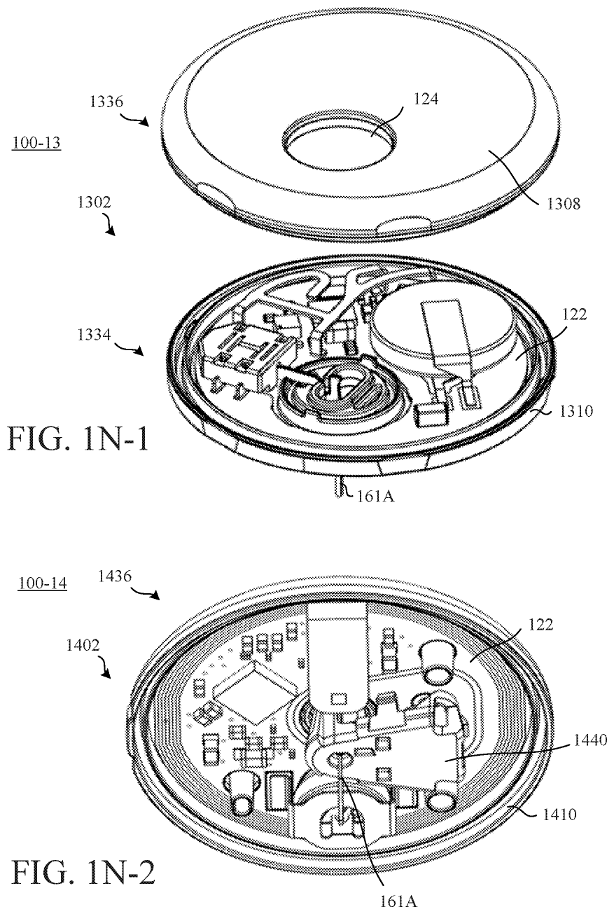

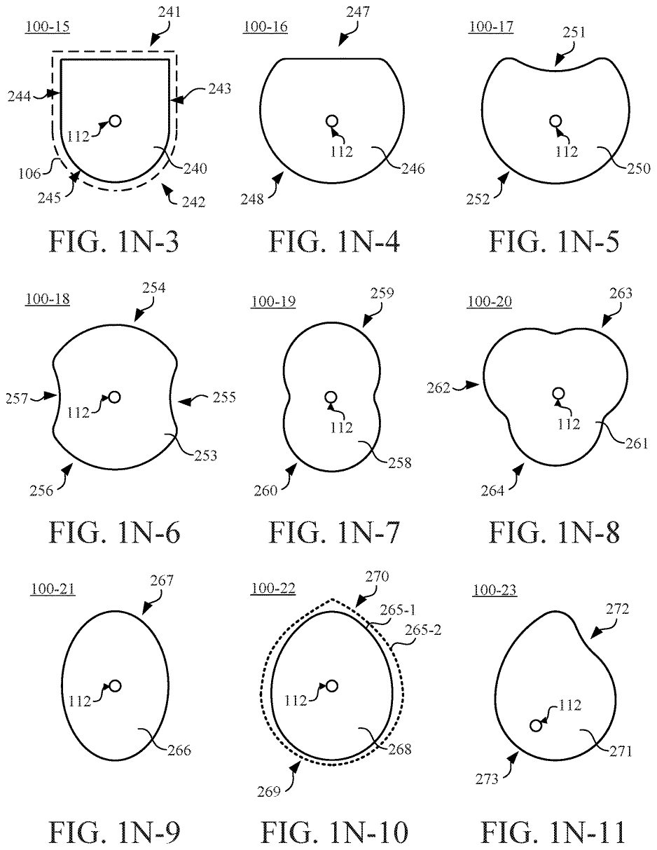

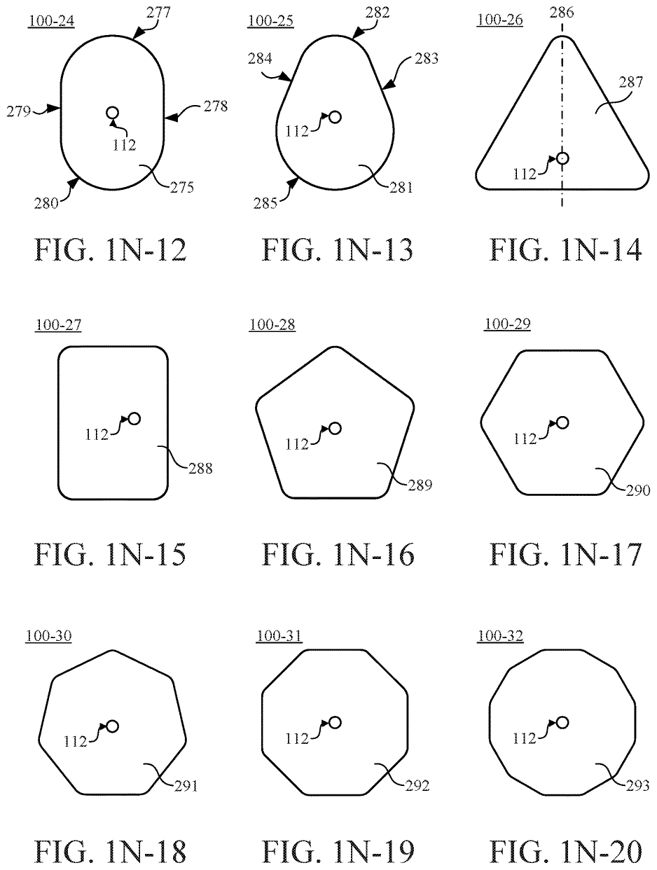

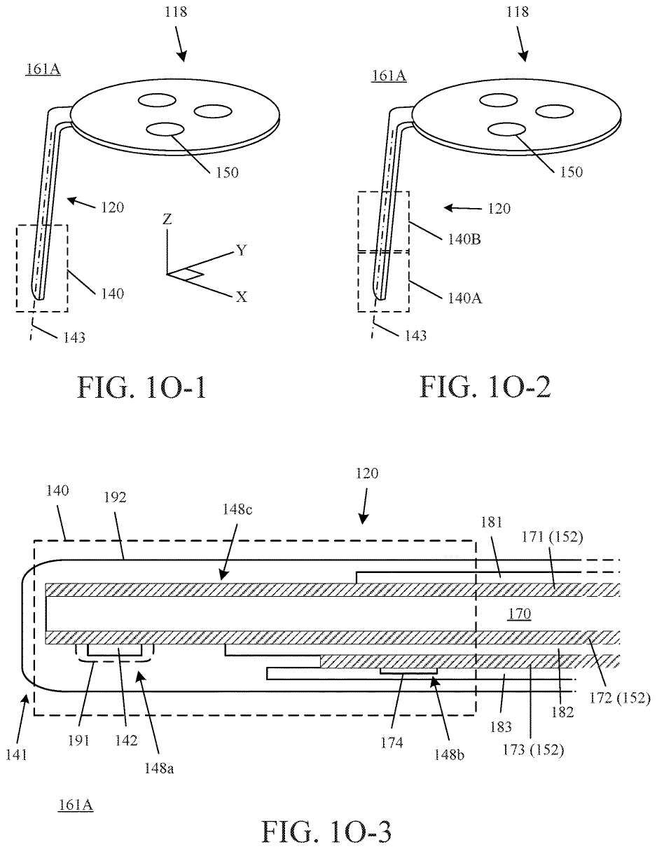

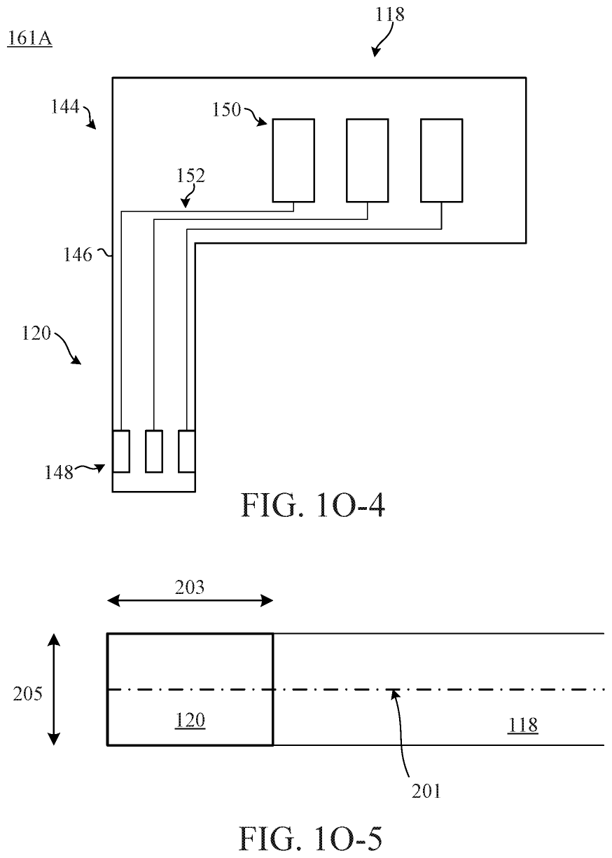

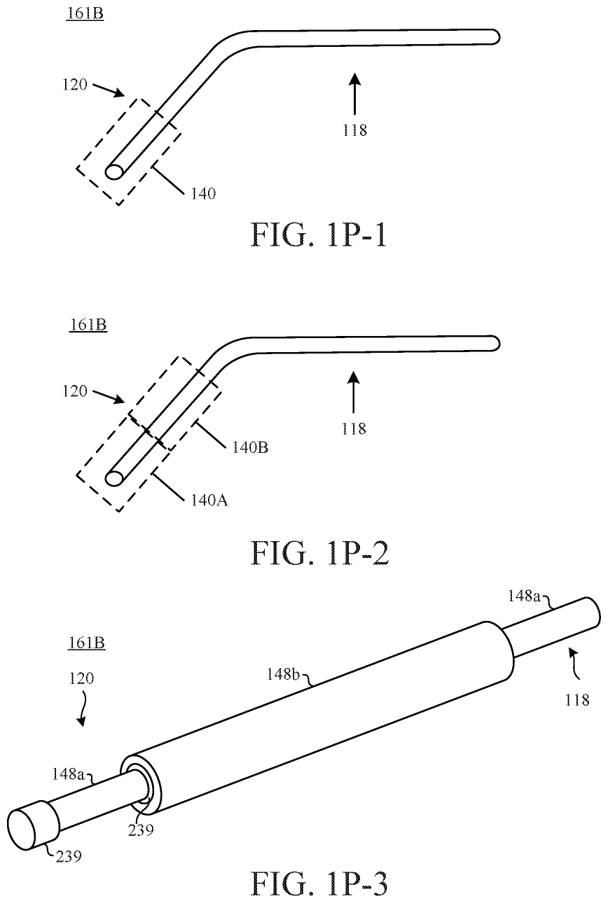

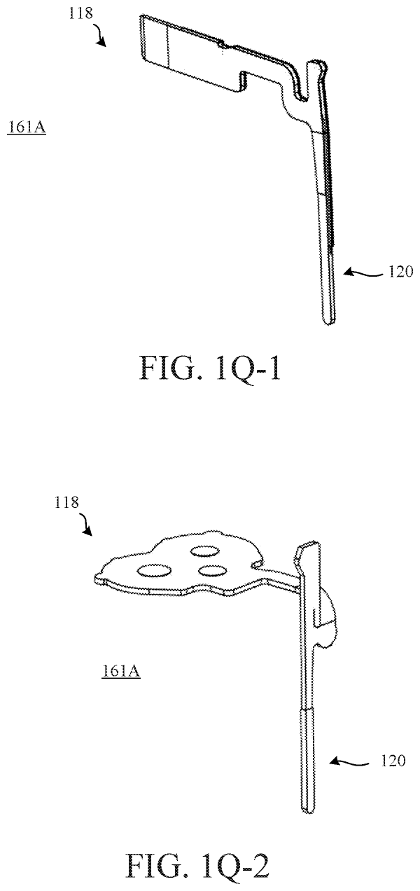

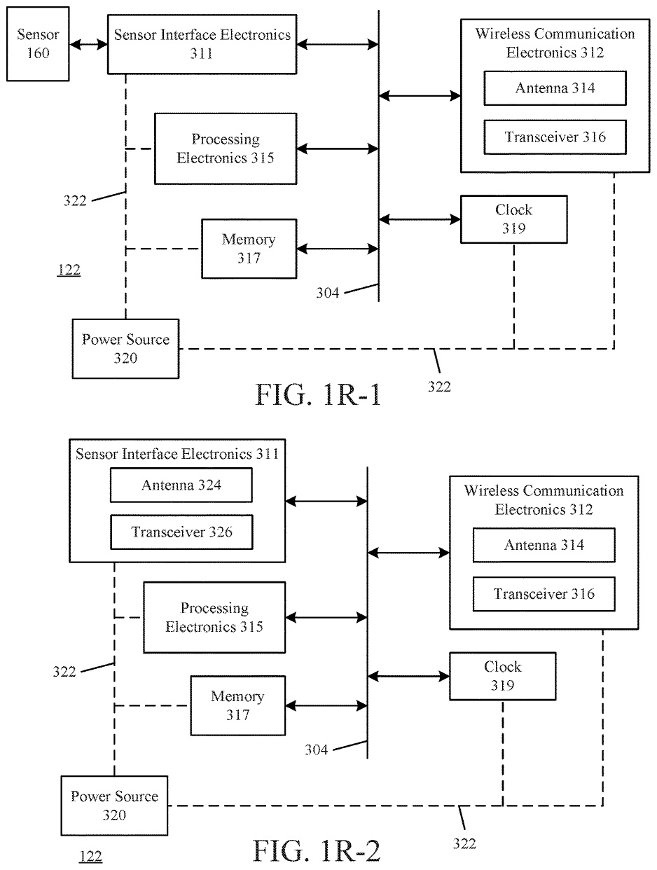

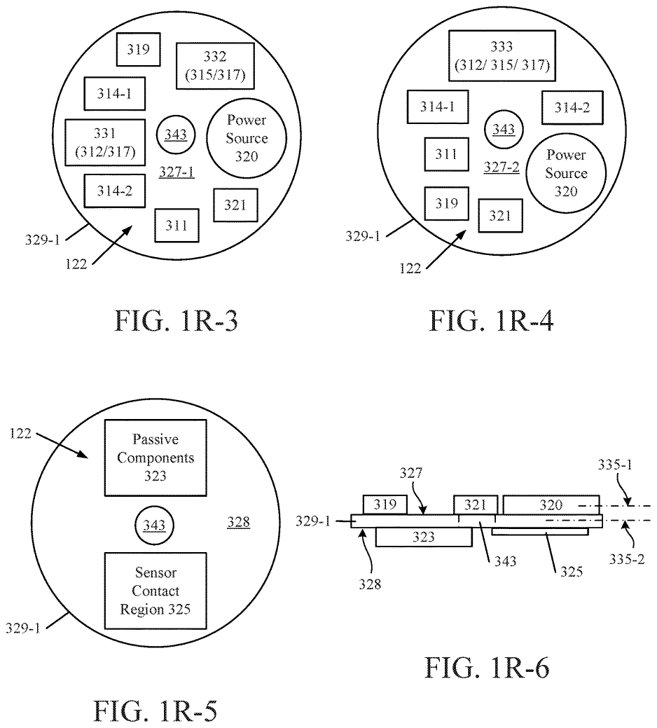

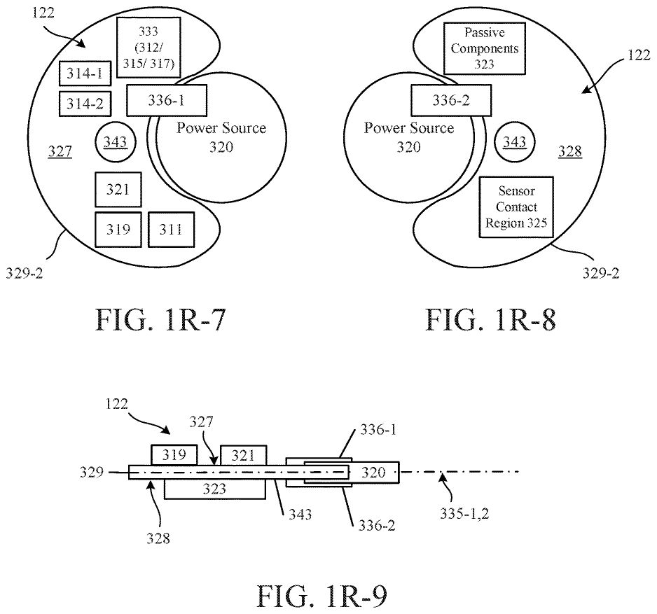

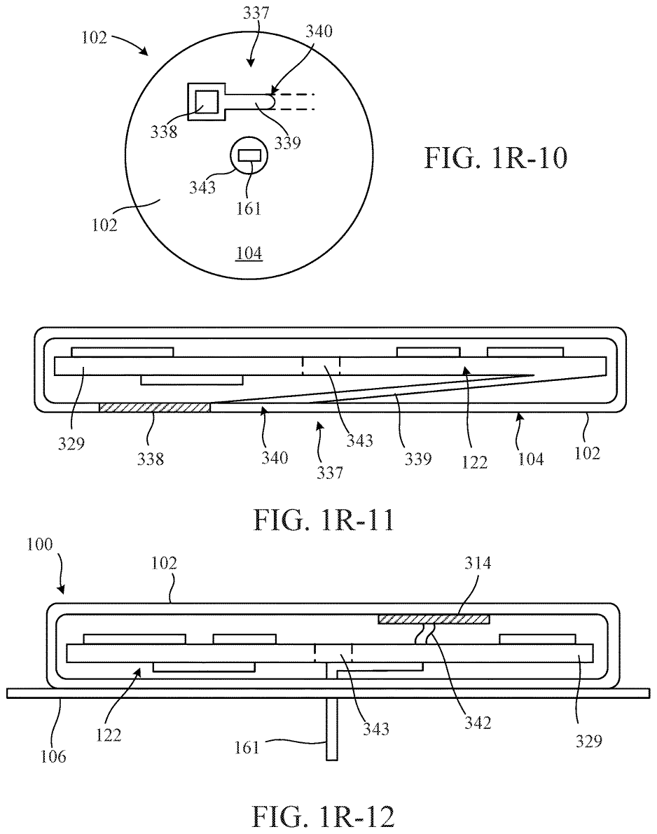

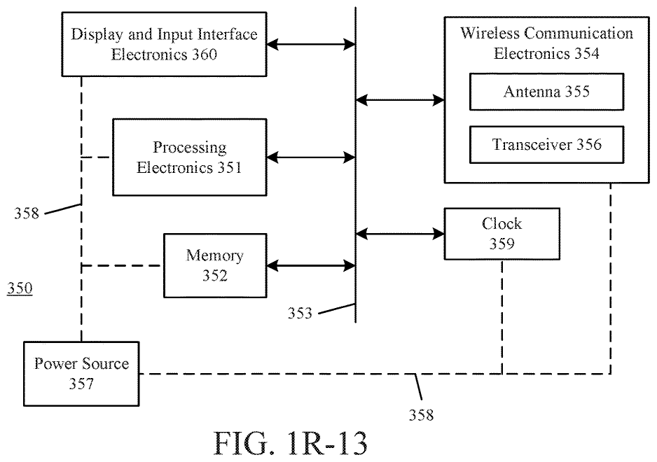

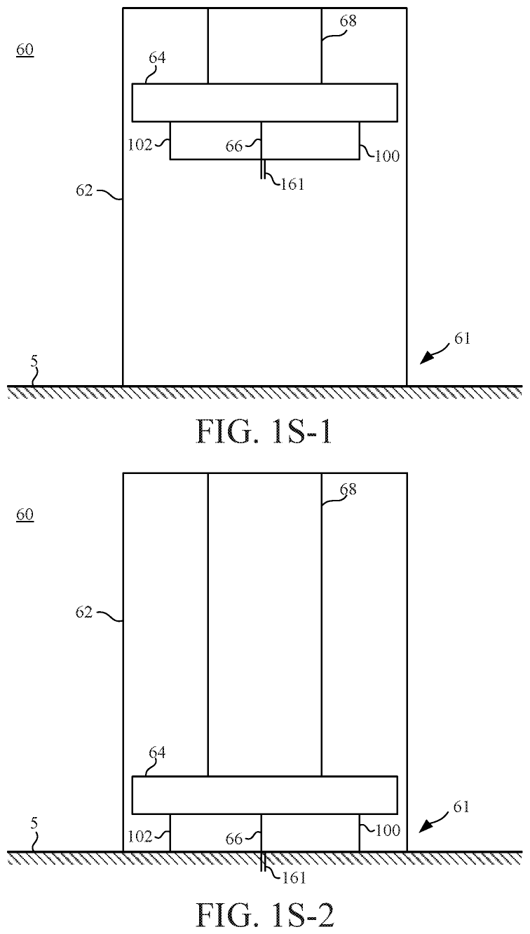

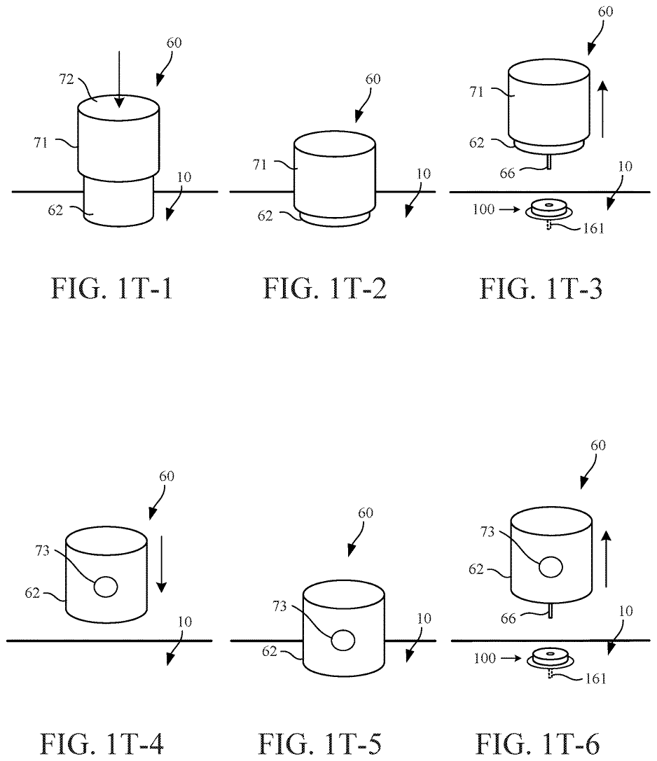

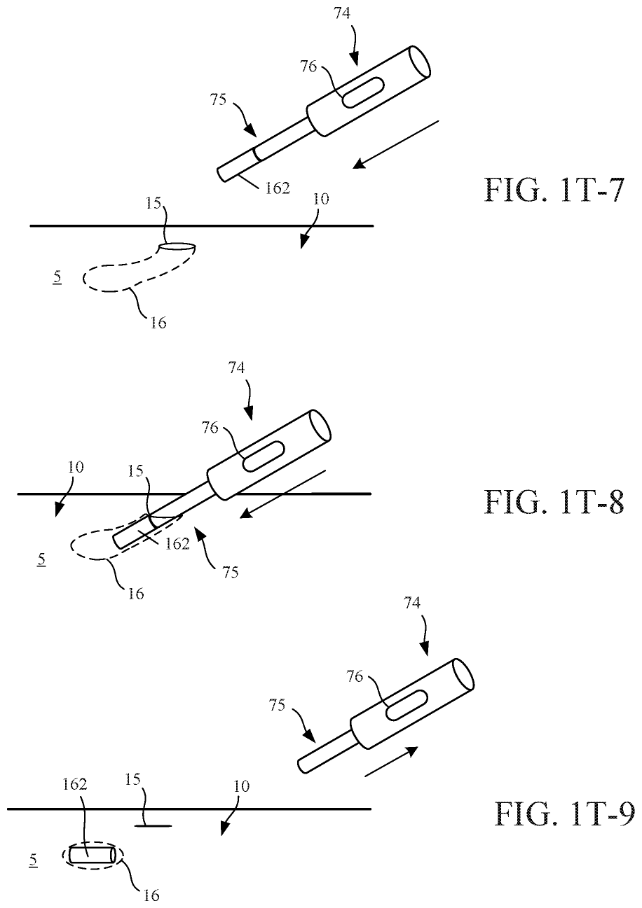

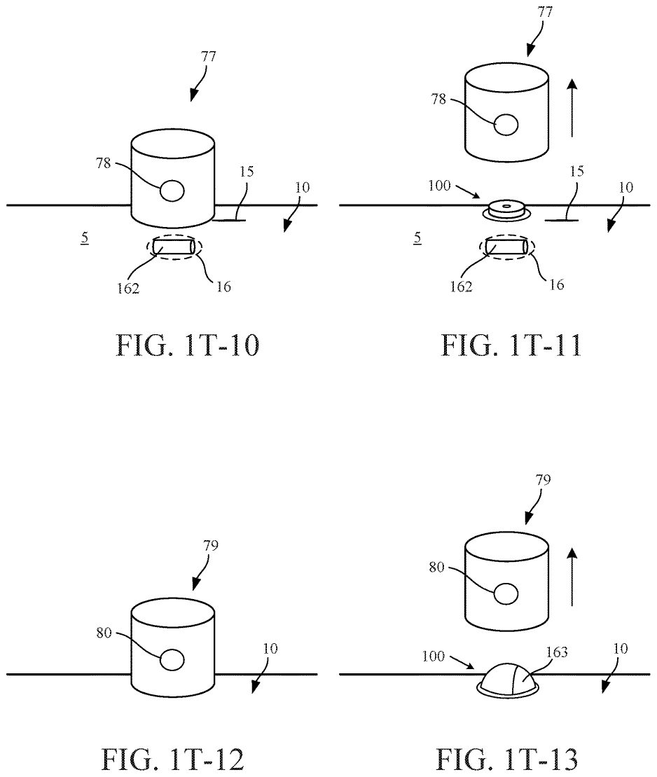

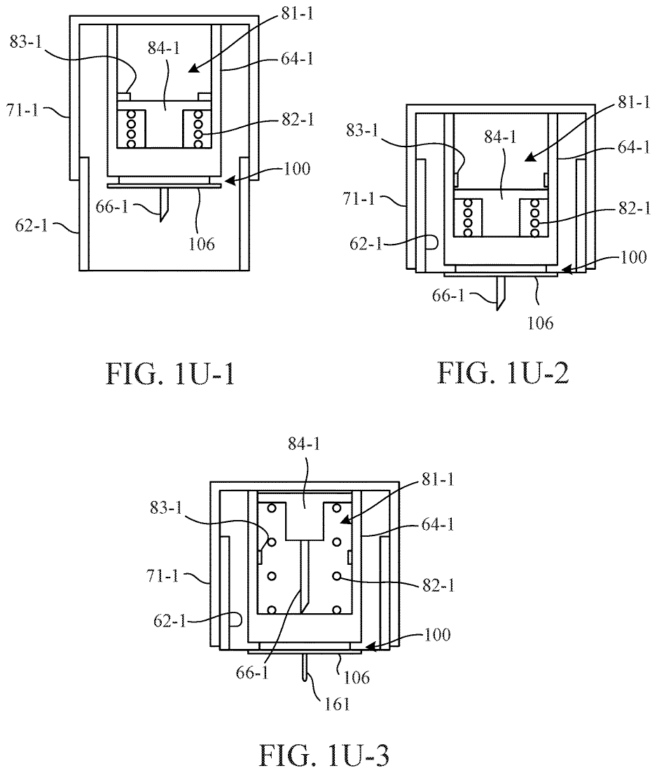

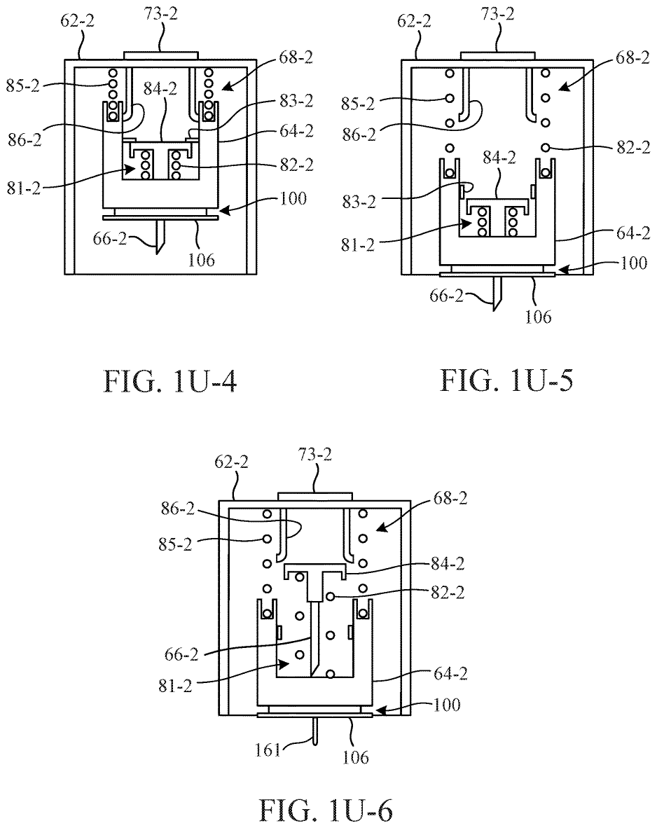

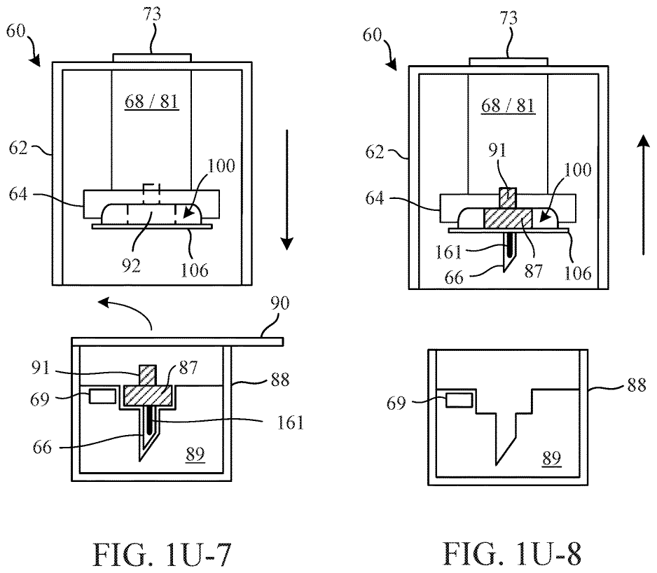

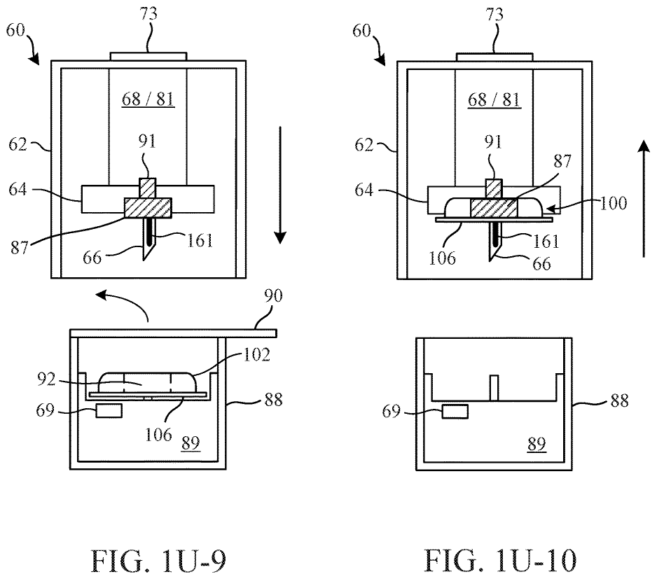

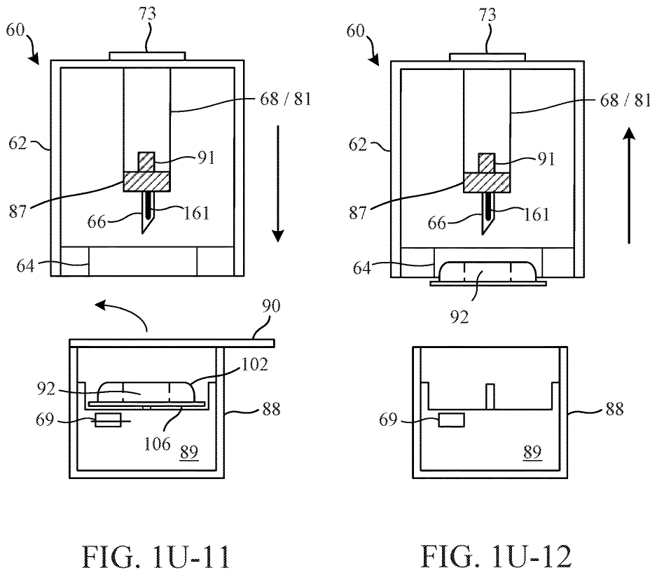

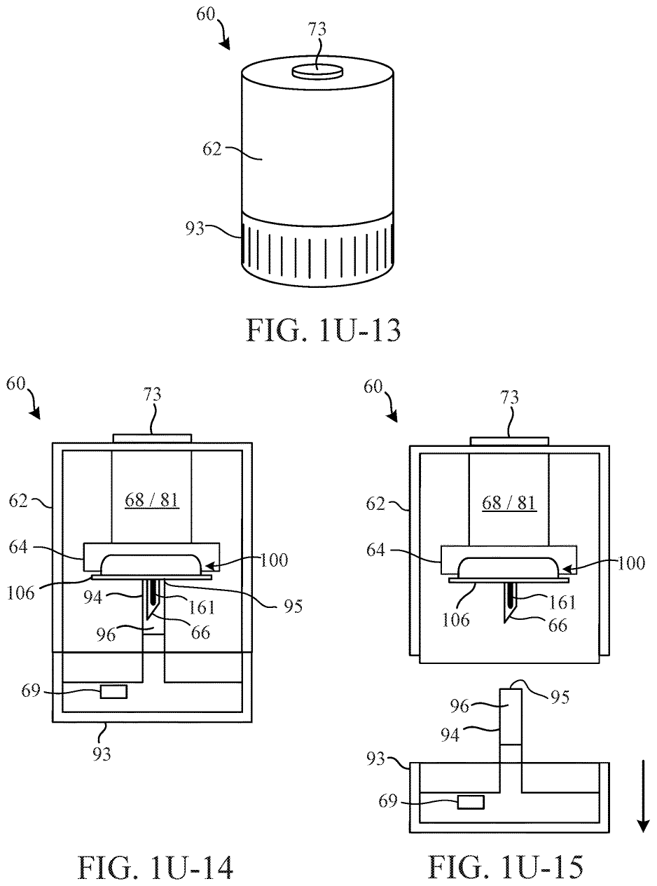

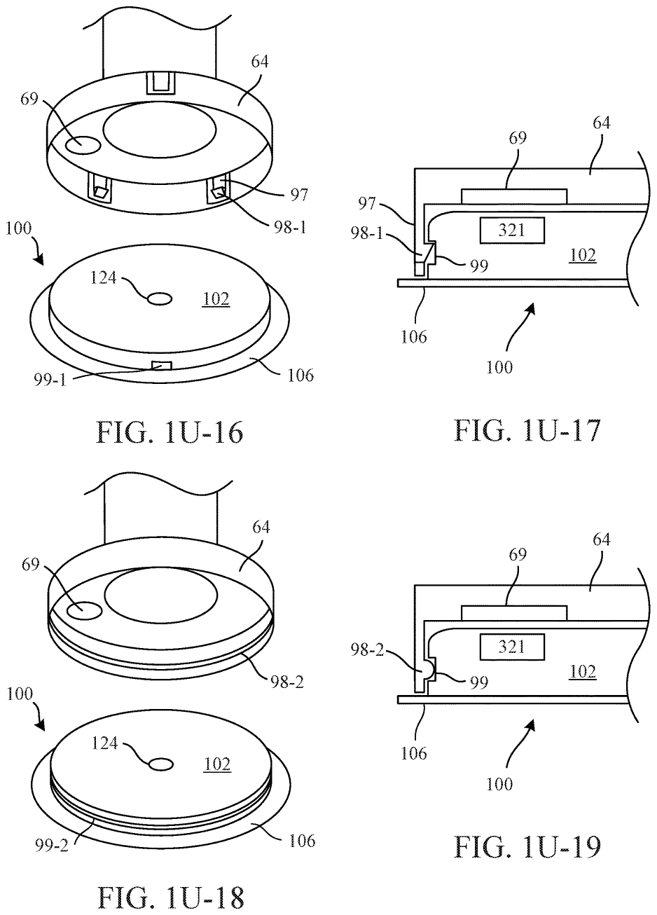

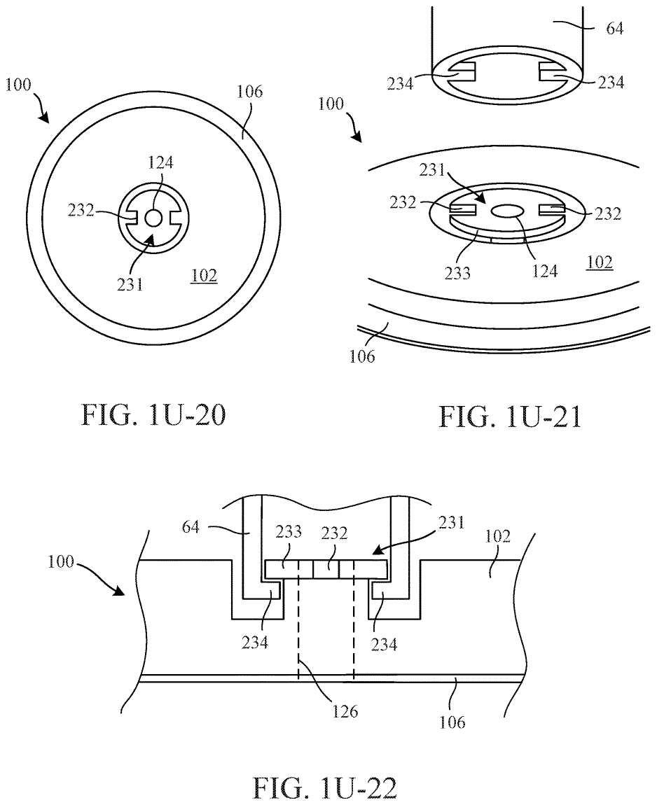

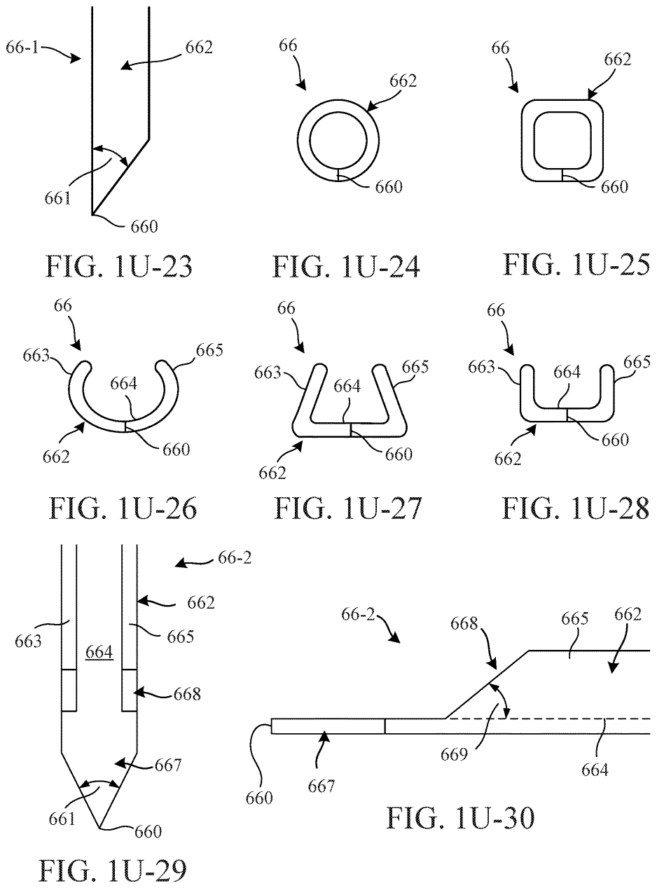

Referring to FIG. 1A, an analyte monitoring system 10 is shown according to an embodiment. In this embodiment, the analyte monitoring system 10 is a glucose monitoring system. In particular, the analyte monitoring system 10 is configured to monitor glucose levels of the user (e.g., a patient). In other examples, the analyte monitoring system 10 may be configured to monitor other analytes (additionally or alternatively to glucose), as described herein.

The analyte monitoring system 10 comprises an on-body device (OBD) 100. OBD 100 may be the on-body device of any of the embodiments described herein. For example, OBD 100 may be OBD 100-1 shown in FIGS. 1B-1 to 1B-3. OBD 100 may have various modifications such as described in relation to OBDs 100-1 through 100-105 and other embodiments described herein. As a result, each of the embodiments of on-body devices described herein may be provided as part of an analyte monitoring system 10, and the analyte monitoring system 10 may be referred to as an on-body analyte monitoring system.

OBD 100 operates in conjunction with a sensor 160 configured to detect an analyte or measure a level of the analyte, also referred to as an analyte sensor. The analyte sensor may be the analyte sensor of any of the embodiments described herein. The analyte sensor 160 can be a partially implantable sensor 161 (also referred to as a partially in vivo sensor or partially insertable sensor), a wholly implantable sensor 162 (also referred to as a wholly in vivo sensor), a wholly ex vivo sensor 163 (can also be referred to as wholly non-implantable sensor) as described herein, and otherwise within the scope of the present description. For example, the analyte sensor may be the analyte sensor 161A described with reference to FIGS. 1O-1 to 1O-4, 1Q-1, and 1Q-2. The analyte sensor may be the analyte sensor 161B described with reference to FIGS. 1P-1 to 1P-3. The analyte sensor may be the analyte sensor 162 described with reference to FIG. 1B-4. The analyte sensor may be the analyte sensor 163 described with reference to FIGS. 1T-12 to 1T-13. As a result, each of the embodiments of analyte sensors described herein may be provided with OBD 100 as part of the analyte monitoring system 10. OBD 100 can include the partially implantable sensor 161 in embodiments utilizing such. OBD 100 can include the wholly ex vivo 163 in embodiments utilizing such. OBD 100 can operate in conjunction with a wholly implantable sensor in embodiments utilizing such. Accordingly, OBD 100 is referred to as generally operating in conjunction with sensor 160, which for this reason is indicated in dashed line in FIG. 1A.

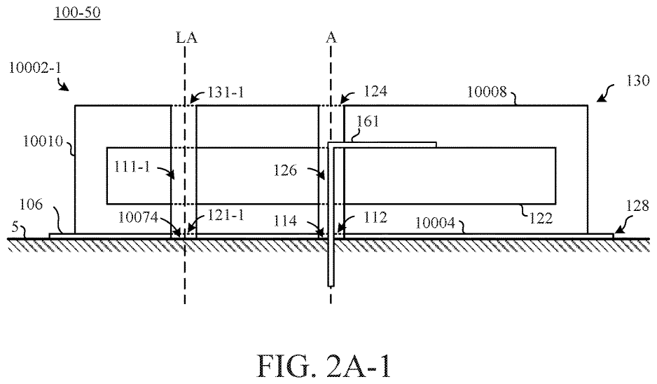

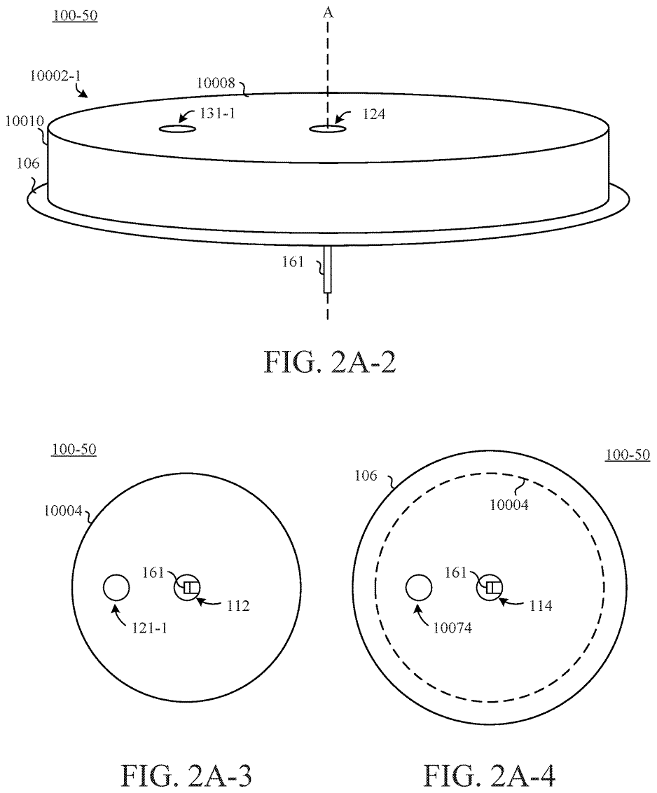

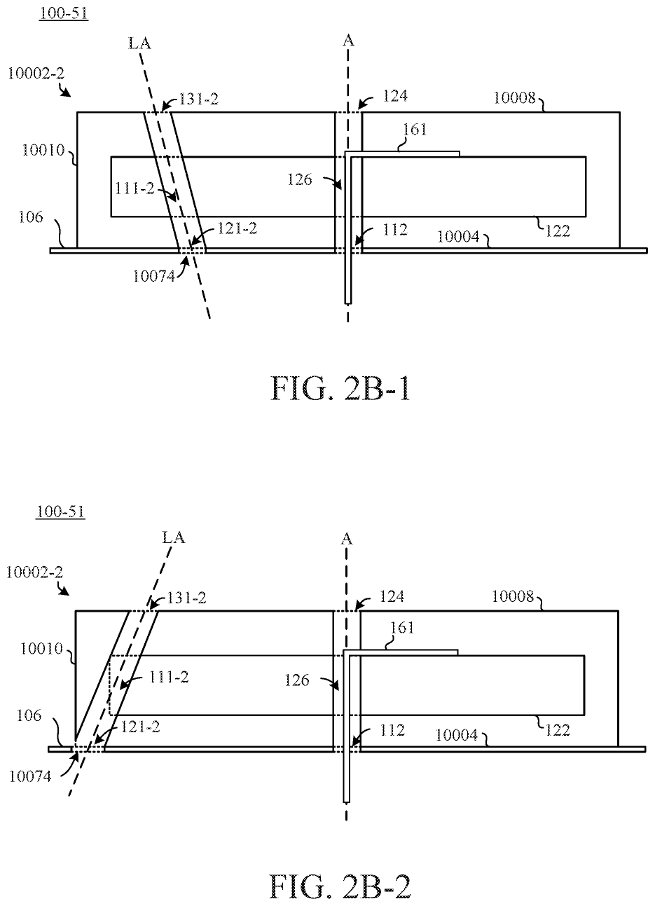

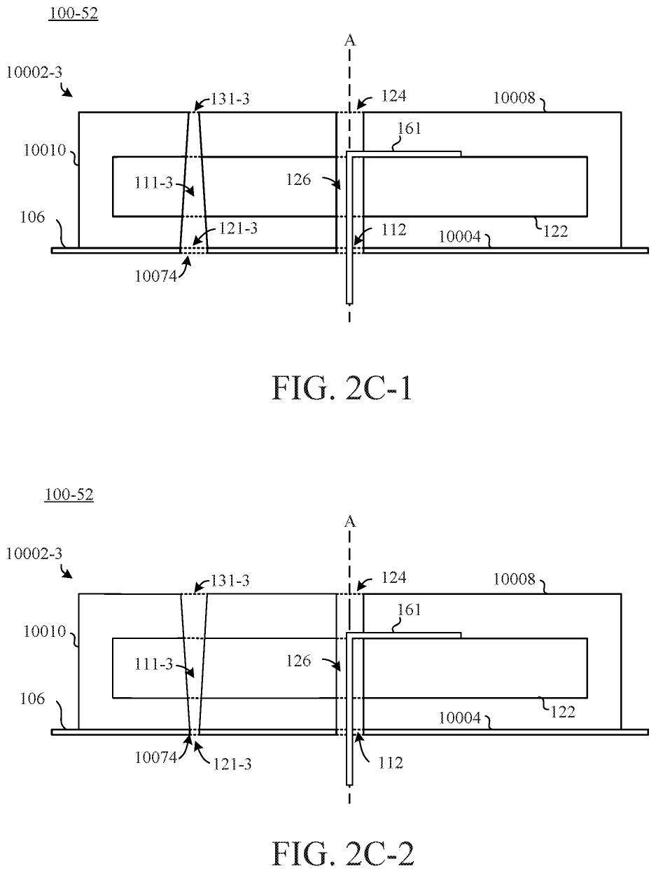

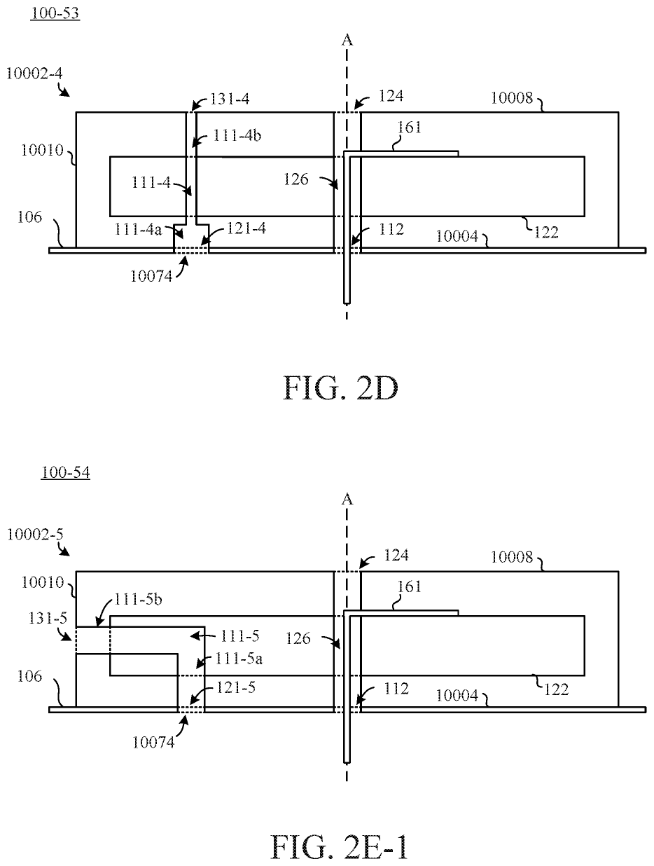

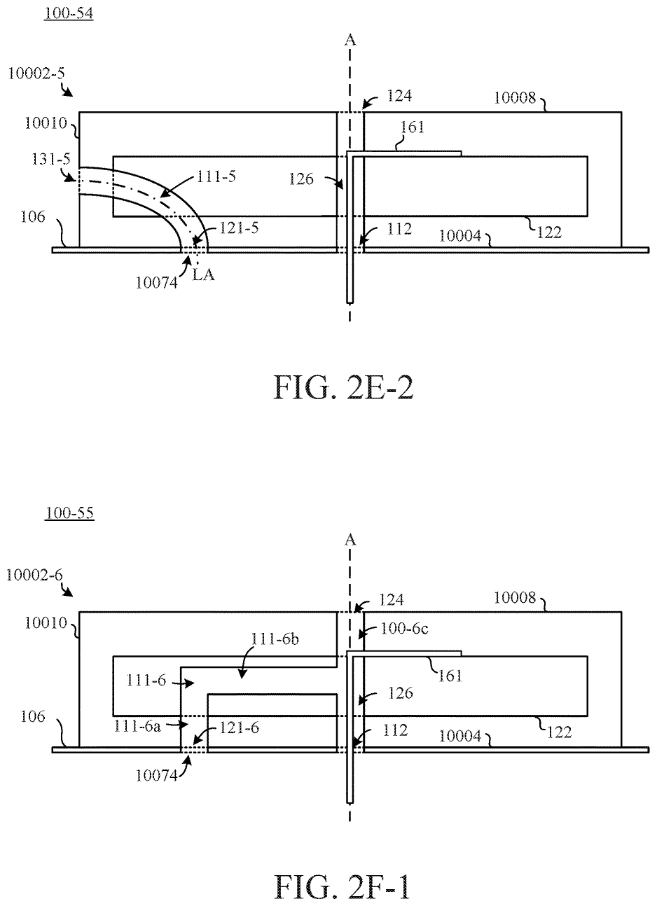

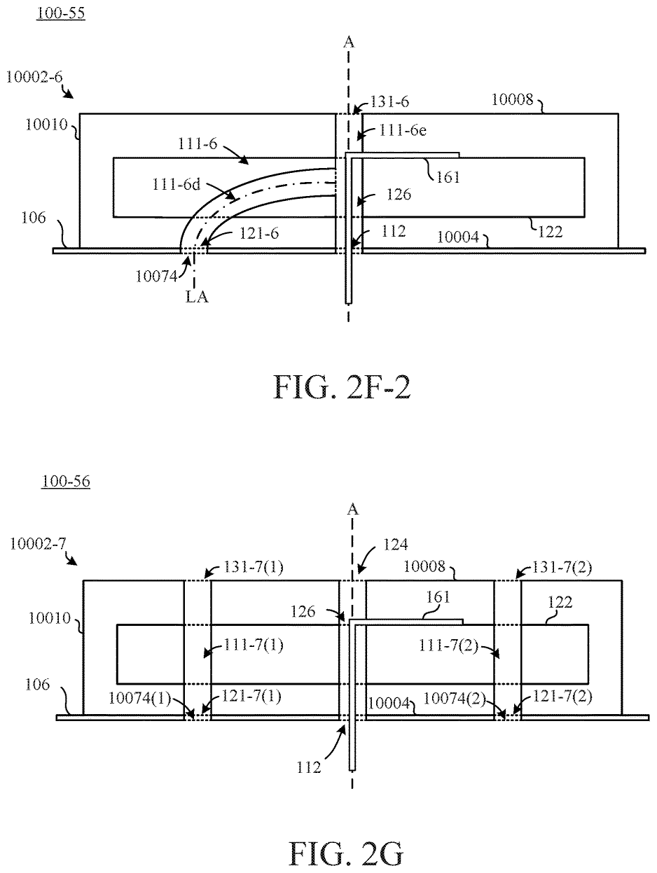

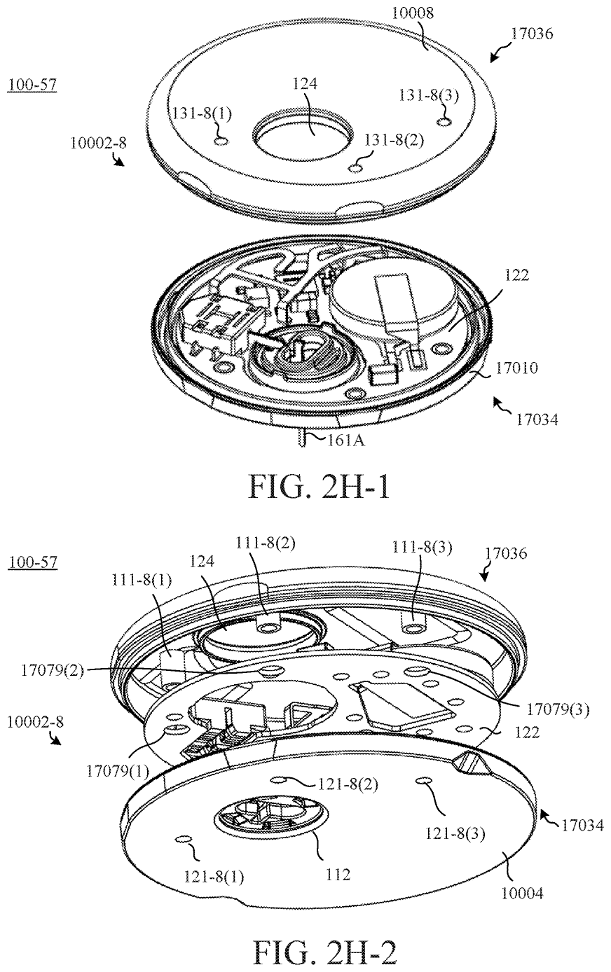

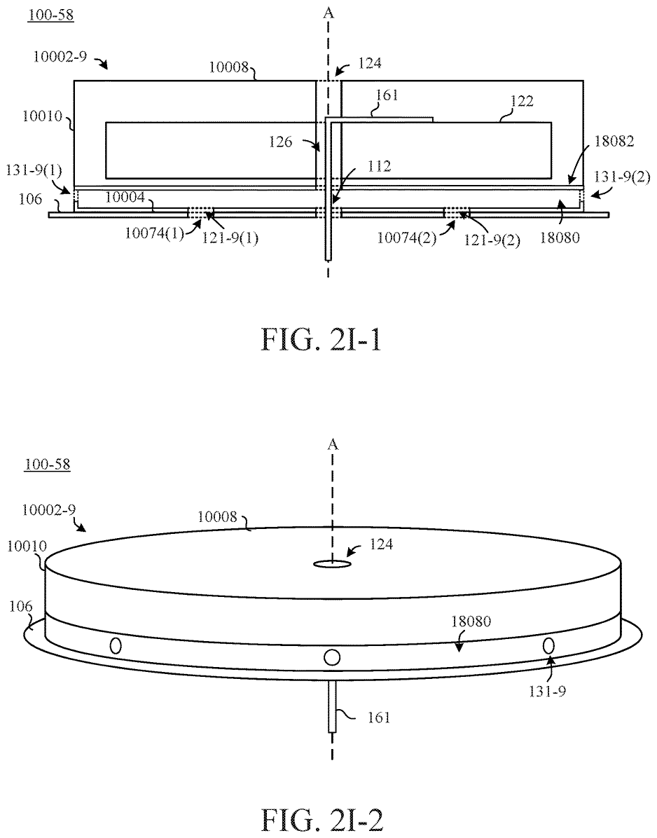

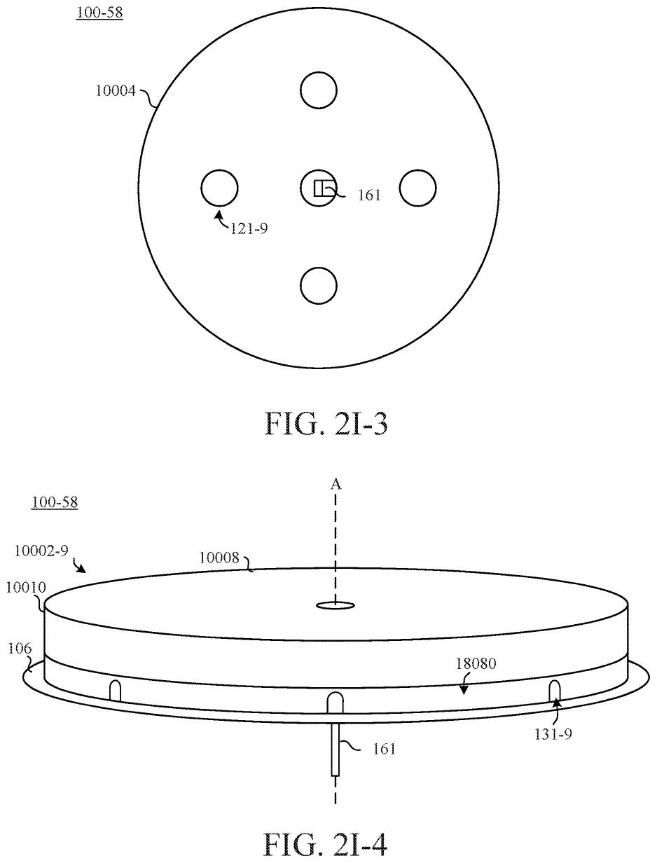

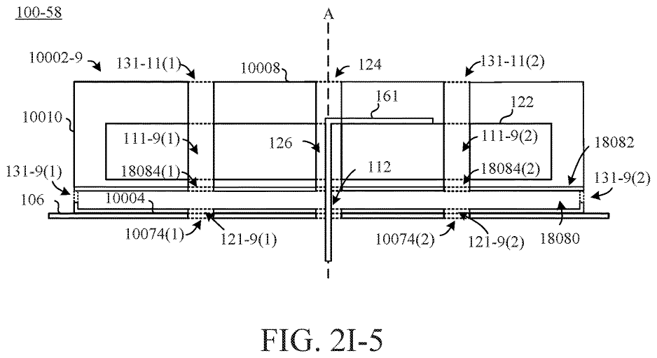

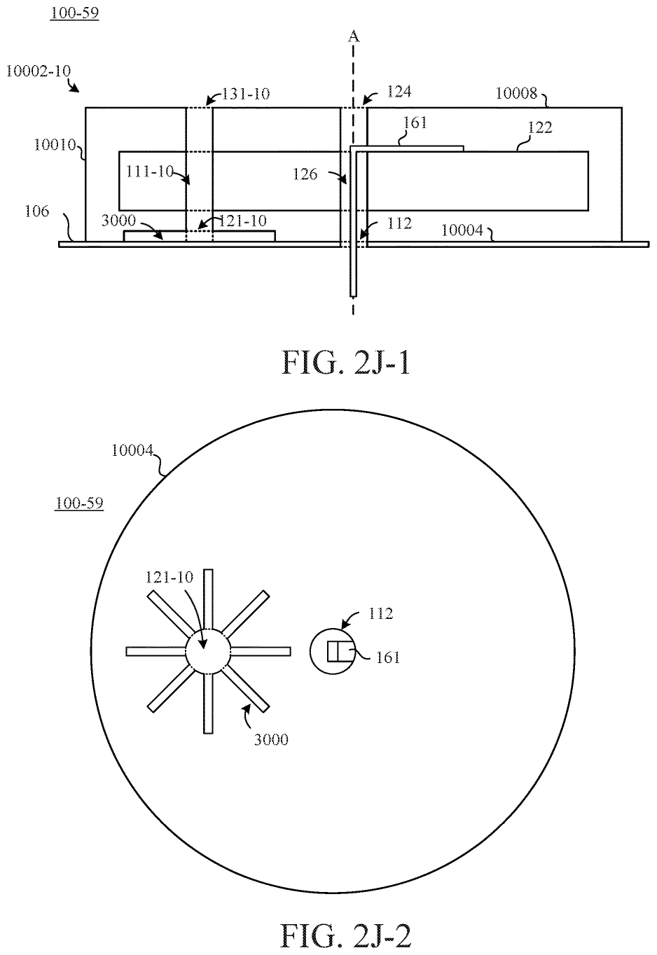

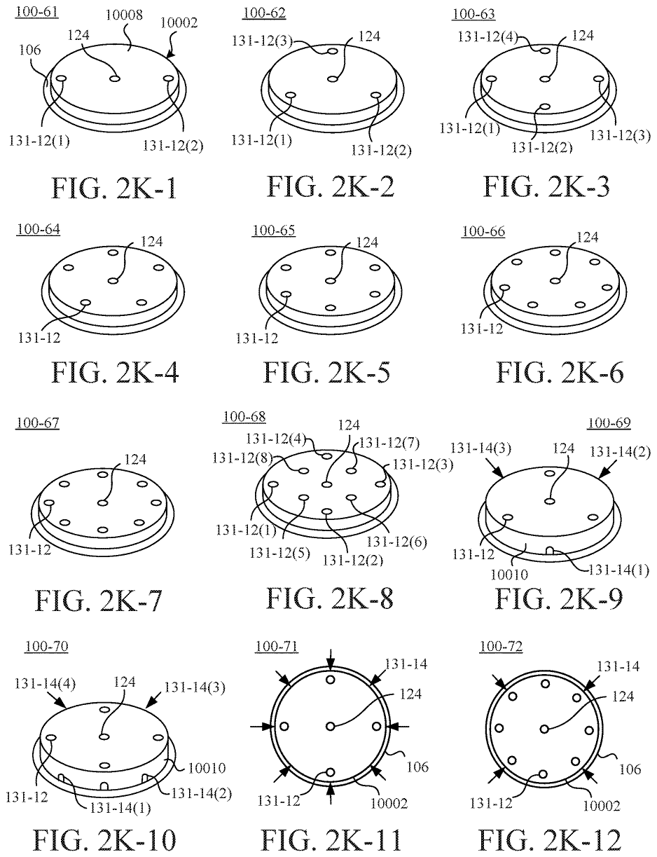

OBD 100 also comprises sensor electronics, which comprise one or more components for operation of OBD 100. The sensor electronics may be the sensor electronics of any of the embodiments described herein. For example, the sensor electronics may be the sensor electronics 122 shown in FIGS. 1R-1 and 1R-2. The sensor electronics may have various modifications such as described in relation to the sensor electronics 122 of FIGS. 1R-1 and 1R-2 or other embodiments. As a result, each of the embodiments of sensor electronics described herein may be provided in OBD 100 as part of the analyte monitoring system 10.

The sensor electronics are configured to interface with, and in some embodiments operate, the analyte sensor. Specifically, the sensor electronics are configured to obtain a signal from the analyte sensor, which corresponds to an analyte level of the user. In this manner, the sensor electronics are in communication with the analyte sensor. In some examples, the sensor electronics are configured to process the signal from the analyte sensor to provide data corresponding to the signal. For example, the data may comprise a digital representation of the signal. For example, the processing may comprise converting an analog signal from the analyte sensor to digital data. In this embodiment, the sensor electronics are configured to generate data that includes at least one value based on the signal received from the analyte sensor. The value may be an unconverted value (e.g., a current or voltage measurement based on the signal obtained by the analyte sensor). In other examples, the value may be a converted value (e.g., an analyte concentration value based on the current/voltage measurement).

The analyte monitoring system 10 also comprises a receiver unit 20. In this embodiment, the receiver unit 20 is a mobile device. In particular, the receiver unit 20 is a mobile phone (e.g., a smartphone). In other examples, the receiver unit 20 is a wearable device (e.g., a smartwatch). In other examples, the receiver unit 20 may be a computing device, such as a computer (e.g., a tablet computer, a laptop computer, or a desktop computer) or a processing unit within a vehicle (e.g., a car or a motorcycle). In other examples, the receiver unit 20 is a dedicated reader device for analyte monitoring, for example provided by the manufacturer of OBD 100. In other examples, the receiver unit 20 may be connected to or included as part of a drug delivery device, such as an insulin pump or an insulin pen. In this embodiment, the receiver unit 20 is operated by the user (e.g., the wearer of OBD 100). In other examples, the receiver unit 20 may be operated by a healthcare professional or by carer.

OBD 100 is configured to communicate with the receiver unit 20. Any two or more of the above receiver units can be configured to communicate with the same OBD 100, simultaneously or sequentially. In particular, the one or more receiver unit(s) 20 is (/are) configured to receive data from OBD 100. The received data may be data corresponding to the signal measured by the analyte sensor. In some examples, the data may be an estimated analyte value. In other examples, the data may be raw data (e.g., a current or voltage), which is processed to convert into an analyte value (e.g., by the receiver 20 or other processing device). Accordingly, OBD 100 is configured to transmit the data to the receiver unit 20. OBD 100 may transmit additional data to the receiver unit 20, such as for establishing a communication link or data relating to calibration of the analyte sensor.

In this embodiment, the data is transmitted automatically. For example, user intervention may not be required to transmit the data from OBD 100 to the receiver unit 20. In some examples, the data may be transmitted periodically. For example, the data may be transmitted every minute, every three minutes, every five minutes, every fifteen minutes, or at another time interval. The data may be transmitted when new data is available. For example, when a new value is determined based on the signal from the analyte sensor, the data may be transmitted automatically. Thus, the transmission interval may correspond to the sampling interval. In other examples, multiple measurements may be taken for a single transmission. Thus, data including multiple values may be transmitted in a single transmission. Alternatively, a single value may be determined from the multiple samples, and that single value is used for transmission. For example, only the most recent value may be transmitted. The other samples may be used for redundancy or for reliability or error checks. In other examples, an average value from the multiple samples may be used (e.g., with data smoothing applied). In another example, the data may be transmitted automatically but at unscheduled times, such as upon occurrence of an event (e.g., an alert condition, such as high glucose or low glucose).

In other examples, the data may be transmitted on-demand. For example, the data may be transmitted in response to user request. For example, user intervention may be required to transmit the data from OBD 100 to the receiver unit 20. In some examples, the user may perform an action to cause the transmission of the data. In one example, the action may comprise the user interacting with a user interface (e.g., on the receiver unit 20, such as pressing a button) to cause transmission of the data by sending a request from the receiver unit 20 to OBD 100. In another example, the action may comprise placing the receiver unit 20 in close proximity to OBD 100 to trigger transmission of the data (e.g., by NFC or RFID). In some examples, other types of transmission may be used, where the transmission is triggered on-demand. For example, the on-demand transmission may be via Bluetooth (e.g., Bluetooth Classic) or Bluetooth Low Energy (BLE) (see, e.g., the Bluetooth Core Specification version 6.0 (2024)). In some examples, the transmission may involve transmitting the data upon a request from the receiver unit 20 (and the request may be provided automatically without user interaction).

In some examples, the data is transmitted automatically by default and may additionally be transmitted on-demand in certain situations, such as upon user request. In some examples, the data is transmitted on-demand by default, and may additionally be transmitted automatically in certain situations, such as after a predetermined period without transmission. Any suitable combination of transmission methods is envisaged in some examples.

In this embodiment, OBD 100 is configured to communicate with the receiver unit 20 over a wireless connection. In particular, the wireless connection is via RF communication. More specifically, the wireless connection uses BLE. In other examples, one, two, or three or more wireless protocols may be used, such as Bluetooth (e.g., Bluetooth Classic), Near Field Communication (NFC) (see, e.g., European Computer Manufacturers Association (ECMA) 340 (2024) and ISO/IEC 18092 (2023), and the NFC Forum Specifications), radio frequency identification (RFID) (see, e.g., the International Organization for Standardization (ISO) and the International Electrotechnical Commission (IEC) 18000 Series, incorporating EPC Gen2 in 2014), Institute of Electrical and Electronics Engineers (IEEE) 802.11 (including 802.11a (1999), 802.11g (2003), 802.11n (2009), 802.11ac (2013), 802.11ax (2011), 802.11be (2024); also referred to as Wi-Fi). The wireless connection may be via a wireless personal area network. In particular, the wireless connection may be low power. For example, communication may be using a technology based on IEEE 802.15, such as IEEE 802.15.4 (2024) (e.g., Zigbee). In other examples, the wireless connection may be via IR communication (or other optical communication). In other examples, the communication may be via a wired connection. In some examples, the communication may be over a local connection (e.g., short range communication), such as by direct connection (e.g., BLE). In some examples, a combination of communication protocols may be used. For example, OBD 100 may be configured to communicate via BLE and NFC (e.g., at different times). Different types of receiver unit 20 (such as those discussed above) may use the same communication protocol to communicate with the same OBD 100 or may use different communication protocols. A receiver unit 20 or 30 may be configured to use NFC to communicate an activation command to initiate operation of OBD 100 or to initiate advertising over a different wireless protocol (e.g., Bluetooth or BLE), a pairing command to initiate pairing with OBD 100, and/or authentication information to inform OBD 100 of a parameter for use in secure communication with receiver units 20 and/or 30 and/or to receive such a parameter from OBD 100. In each instance subsequent regular communication between OBD 100 and receiver units 20 and/or 30 may occur using a different wireless communication protocol (e.g., Bluetooth or BLE).

In some examples, the receiver unit 20 is configured to transmit data to OBD 100. For example, the data transmission may be bi-directional (e.g., OBD 100 provides the data to the receiver unit 20 and the receiver unit 20 can provide data to OBD 100). For example, the data transmitted to OBD 100 may be a response such as acknowledgement of receipt of the data from OBD 100. The receiver unit 20 may also transmit other information such as providing identification information for the receiver unit 20, establishing a connection, calibration information, or requesting data transmission. In other examples, the data transmission may be one-way (e.g., OBD 100 provides the data to the receiver unit 20 without receiving a response).

In this embodiment, the receiver unit 20 performs processing of the data received from OBD 100. For example, the receiver unit 20 may convert the received data into analyte values. In particular, the receiver unit 20 may receive raw data (e.g., unconverted data). In other words, the received data may correspond to a value (e.g., in digital form) representing the signal from the analyte sensor (e.g., a current or voltage). The data can then be converted from the signal value into a concentration value of the analyte (e.g., in units of mg/dL or mmol/L). For example, a conversion function may be used to convert the data using a known calculation. The data may also be compensated using temperature. In some examples, the receiver unit 20 may calibrate the data. For example, different analyte sensors may have different sensor sensitivities, resulting in different signal levels for the same analyte concentration. Calibration can be used to adjust the concentration derived from the data to provide a more accurate value. For example, calibration data may be input (e.g., from fingerstick measurements using a blood glucose meter, for example input by a user) and used to calibrate the analyte sensor by updating the calibration function. In other examples, factory calibration may be used, where a calibration function is derived at the time of manufacture. This may be stored in a memory of the sensor electronics for providing to a receiver unit 20 during use. In some examples, the calibration function may be used by a processor of OBD 100 for calibrating the data from the analyte sensor. In some examples, the period of time before activation of OBD 100 may be determined (e.g., determining a “shelf life” of OBD 100 between manufacture and activation of OBD 100) and the data may be adjusted based on the determined period of time. Any of the above adjustment methods can be combined as appropriate. For example, temperature compensation and calibration may be performed on the data and/or with shelf-life compensation, etc.

In some examples, the receiver unit 20 may perform calculations such as determining a rate of change of the analyte level based on the data. For example, this can be determined based on the change between two values of the analyte level at different times (e.g., adjacent points), divided by the time period between the values. Providing processing functionality on the receiver unit 20 reduces the processing requirements on OBD 100, reducing the hardware requirements and reducing power consumption. In other examples, various processing (such as conversion, temperature compensation, calibration, and/or rate of change calculations) may be performed on OBD 100.

In this embodiment, the receiver unit 20 is configured to output information to the user. In particular, the receiver unit 20 is configured to display the information to the user. The receiver unit 20 may comprise a computer program (e.g., a software application) for execution on the receiver unit 20. The computer program may be configured to cause the receiver unit 20 to receive the data. The computer program may also be configured to cause the receiver unit 20 to process the data. The computer program may also be configured to cause the receiver unit 20 to output the information, such as by displaying information on a screen of the receiver unit 20. In some examples, the analyte monitoring system 10 comprises the computer program in isolation of the receiver unit 20 (e.g., without a receiver unit 20). For example, the computer program may be provided and may installed on a receiver unit 20 (e.g. by the user).

In some examples, the information output by the receiver unit 20 comprises analyte data based on the data received from OBD 100. As one example, the receiver unit 20 may display the current analyte level of the user (e.g., the concentration after conversion). In some examples, the receiver unit 20 may also display the rate of change of the analyte level (e.g., in the form of a trend arrow indicating the magnitude and direction of the current rate of change). In some examples, the receiver unit 20 may also display a plurality of values of the analyte level over time (e.g., historical analyte data), such as on a graph. As new data is received, the receiver unit 20 may update the current analyte level, may update the rate of change, and/or may add the new analyte value to the graph (e.g., alongside the historical analyte data).

In some examples, the receiver unit 20 may also output notifications, such as alerts. This may include displaying an alert notification, such as an alert screen or pop-up window. This may also include auditory and/or vibratory notifications. Alerts may include analyte-related alerts, such as if the analyte level is outside (e.g., above or below) threshold levels, or if the rate of change is outside threshold levels (e.g., rising or falling too quickly). The analyte-related alerts may include predictive alerts, such as predicting that the analyte level will be above or below the threshold level within a predetermined amount of time. Alerts may also include system alerts, such as alerts related to a transmission signal issue or a sensor issue. In response to detecting an alert, the receiver unit 20 may display an alert notification, output an audible alert, and/or output a vibratory alert. The receiver unit 20 may also output notifications that are not alerts, such as to provide analytics information or other information.

In this embodiment, the analyte monitoring system 10 also comprises a secondary receiver unit 30. The secondary receiver unit 30 may be a further receiver unit in addition to the receiver unit 20. For example, the receiver unit 20 may thus be referred to as a primary receiver unit. In other examples, the secondary receiver unit 30 is not provided.

In this embodiment, the secondary receiver unit 30 is a wearable device. In particular, the secondary receiver unit 30 is a smartwatch. For example, the secondary receiver unit 30 may be a mobile device (such as a mobile phone), a computing device (such as a computer or processing unit within a vehicle), a dedicated reader device for analyte monitoring, or connected to or part of a drug delivery device. In this embodiment, the secondary receiver unit 30 is operated by the user. In other examples, the secondary receiver unit 30 may be operated by a healthcare professional or by a caregiver of the user (e.g., the user being the wearer of OBD 100).

In this embodiment, OBD 100 is configured to communicate with the secondary receiver unit 30. The secondary receiver unit 30 is configured to receive data from OBD 100 (e.g., the data corresponding to the signal measured by the analyte sensor), in a similar manner to the receiver unit 20. Features of the transmission between OBD 100 and the receiver unit 20 may readily be applied to transmission between OBD 100 and the secondary receiver unit 30. In this embodiment, OBD 100 communicates with the secondary receiver unit 30 in a similar manner to the receiver unit 20, in particular by wireless communication (BLE in this case). In other examples, other communication protocols may be used as described above. In this manner, OBD 100 may be configured to communicate directly with multiple devices. The secondary receiver unit 30 may also transmit data to OBD 100 (bi-directional), or the communication may be one way from OBD 100 to the secondary receiver unit 30.

In other examples, the secondary receiver unit 30 may not communicate directly with OBD 100. In particular, the secondary receiver unit 30 may communicate indirectly with OBD 100. For example, the secondary receiver unit 30 may be configured to communicate with the receiver unit 20 (or other component) to receive data from OBD 100 via the receiver unit 20. This may reduce the transmission requirements for OBD 100, thereby reducing power consumption. Receiver units 20 and 30 may also communicate with one another without involving OBD 100. For example, an app on receiver unit 20 can send sensor-context information to the secondary receiver unit 30 configured as a drug delivery pump so that the pump can connect to OBD 100, as the pump may lack the requisite wireless communication circuitry (e.g., NFC capability) to scan the OBD 100 and obtain authentication data for pairing (e.g., via Bluetooth or BLE). In a multi-device connected system, if one receiver unit 20 or 30 receives and dismisses an alert, that dismissal may be communicated to the other receiver(s) 20 and/or 30 so they do not continue to sound the alarm. System 100 is capable of accommodating one, two, three or more receiver units 20 and one, two, three or more secondary receiver units 30 concurrently.

The secondary receiver unit 30 may perform similar processing to the receiver unit 20, such as converting the received data into analyte data and/or determining a rate of change. The secondary receiver unit 30 may also output information to the user in a similar manner to the receiver unit 20, such as displaying information comprising analyte data and/or outputting notifications such as alerts.

In this embodiment, the receiver unit 20 is configured to transmit data via a network 40 to a server 50. For example, the network 40 may include the internet. In particular, the receiver unit 20 is configured to transmit the data over the internet. For example, the receiver unit 20 may connect to the internet, such as by a Wi-Fi or cellular wireless connection. The data may be transmitted over the internet using various forms of data transmission, such as wired connections, wireless connections, and/or satellite connections. For example, other components such as routers and switches may be utilized. In other examples, other communication methods may be used. In some examples, the secondary receiver unit 30 is additionally or alternatively connected to the network 40 and may be configured to transmit data to the server 50.

The server 50 may be an internet server (e.g., a cloud server). The server 50 may be operated by the manufacturer of OBD 100 and/or the receiver unit 20. In this embodiment, the receiver unit 20 is configured to transmit analyte data to the server 50 via the network 40. The server 50 is configured to process the received analyte data and can provide analysis information, such as trends and/or reports for the user. The analysis information may include information such as average analyte values over a time period and/or time in range (time spent within threshold levels). In some examples, the server 50 is then configured to return the analysis information to the receiver unit 20 via the network 40, which can then be output to the user. In other examples, the receiver unit 20 does not communicate with a server 50. In some examples, the analysis information may be obtained by processing performed on the receiver unit 20 (or other local device). In other examples, the server 50 (and the network 40) may not be provided.

In some examples, the receiver unit 20 may be configured to communicate with a further receiver unit over the network 40. The further receiver unit may be a further receiver unit in addition to the receiver unit 20 and optionally in addition to the secondary receiver unit 30 (or instead of the secondary receiver unit 30). For example, the further receiver unit may be a desktop computer. In other examples, the further receiver unit may be similar to the receiver unit 20 or the secondary receiver unit 30 and may be any of the devices described in relation to the receiver unit 20 or the secondary receiver unit 30.

For example, the receiver unit 20 may be configured to transmit analyte data to the further receiver unit via the network 40 (e.g., the internet). In some examples, this data may include analysis information obtained from the receiver unit 20 or from the server 50. In other examples, the server 50 communicates directly with the further receiver unit. For example, the server 50 may provide the analysis information to the further receiver unit in addition to (or instead of) providing this to the receiver unit 20.

In some examples, the further receiver unit is not operated by the user. For example, the further receiver unit may be operated by a third party. In particular, the further receiver unit may be operated by the user's healthcare professional. This may allow the healthcare professional to view the data (such as the analyte data and analysis information). In other examples, the further receiver unit may be operated by a designated contact or carer, such as a family member or friend. This may allow the designated contact to follow the user's analyte monitoring. For example, the designated contact may be notified, such as if the user's analyte level drops to a critical level, thereby providing an emergency notification system. This is particularly useful in situations where the user is a child and the analyte levels can be monitored by a designated contact (e.g., a parent). In other examples, the further receiver unit may not be provided.

### On-Body Device

The following section describes various embodiments of an on-body device 100. This includes a description of embodiments of on-body devices 100-1 to 100-32 described with reference to FIGS. 1B-1 to 1N-20. The terms “on-body device 100” and “OBD 100” each refers, collectively to all iterations of on-body devices 100 described herein, including OBDs 100-1 to 100-32 in this section and OBDs 100-50 to 100-105 described thereafter. Accordingly, features of the on-body device 100 described herein may be applicable to any of OBDs 100-1 to 100-32 and OBDs 100-50 to 100-105.

### On-Body Devices 100-1

Referring to FIGS. 1B-1 to 1B-4, an OBD 100-1 is shown according to an embodiment. OBD 100-1 is configured for placement on the body 5 of the user (e.g., a patient). In particular, OBD 100-1 is configured to be placed on the skin of the user. In some examples, OBD 100-1 may be referred to as an on-body device. In some examples, OBD 100-1 may be referred to as a sensor electronics unit or a sensor electronics device. In some examples, OBD 100-1 may be referred to as a sensor control unit or a sensor control device. In some examples, OBD 100-1 may be referred to as an analyte monitoring device.

OBD 100-1 is configured for monitoring an analyte level of the user. In this embodiment, the monitored analyte is glucose. Thus, OBD 100-1 may be referred to as a glucose monitoring device. However, in other examples, other analytes (or combinations of different analytes) may be monitored, as described herein.

FIG. 1B-1 shows a cross-sectional view of OBD 100-1. OBD 100-1 can operate in conjunction with various types of sensors. In this embodiment, OBD 100-1 operates in conjunction with a partially implantable sensor 161. The analyte sensor 161 is configured to be inserted into the body of the user. In particular, the analyte sensor 161 is configured to be in contact with bodily fluid (e.g., interstitial fluid, dermal fluid, or blood). The analyte sensor 161 is configured to measure a signal corresponding to the analyte level of the user. In particular, the analyte sensor generates an analog signal (e.g., a voltage or current) related to the concentration of the analyte in the bodily fluid. Further details of the analyte sensor 161 are described herein, including variants 161A and 161B.

As shown, OBD 100-1 comprises a housing 102. The housing 102 is configured to retain various components of OBD 100-1, such as electronics 122 that may be used to control the sensor and process the resulting signal for monitoring the analyte. As a result, the housing 102 may be referred to as an electronics housing or a sensor electronics housing. In some examples, the housing 102 may be referred to as an enclosure. The electronics 122 may also be used to transmit data associated with the sensor signal to a receiver unit. For example, OBD 100-1 may be configured to transmit data to a receiver unit 20 (or secondary receiver unit 30) such as described in relation to FIG. 1A. As a result, the housing 102 may be referred to as a transmitter housing. OBD 100-1 may also be referred to as a transmitter unit. Even if referred to as such, OBD 100-1 can also have the capability to receive data and information from receiver units 20 and 30.

In this embodiment, the housing 102 comprises a base surface 104. The base surface 104 is a surface of the housing 102. In particular, the base surface 104 is an exterior surface of the housing 102 (e.g., an outer surface, or an external surface). The base surface 104 may be arranged at the base of the housing 102. The housing 102 may comprise a base. The base may be a base portion of the housing 102. The base surface 104 is arranged at the bottom of the housing 102. The base surface 104 may alternatively be referred to as a bottom surface, a lower surface, a proximal surface, an adhesive-contacting surface, or a skin-facing surface. The base surface 104 is configured to be positioned towards the skin of the user. Specifically, when OBD 100-1 is placed on the skin of the user, the base surface 104 faces towards the skin. The presence of a base or base surface does not imply any particular manner of construction of OBD 100.

As shown in FIG. 1B-1, a central axis A is shown through OBD 100-1. The central axis A passes through the center of OBD 100-1. The central axis A is perpendicular to the base surface 104. When OBD 100-1 is placed on the skin, the central axis A is perpendicular to the skin. For example, when the base surface 104 is arranged horizontally, the central axis A is arranged vertically, as shown in FIG. 1B-1.

The central axis A extends in a proximal-distal axis. The proximal-distal axis extends between the proximal direction and the distal direction. Thus, the proximal direction and the distal direction are both parallel to the central axis A. The proximal direction is opposite to the distal direction. The proximal direction may correspond to the direction towards the base surface 104 (e.g., starting from the center of the housing 102). The distal direction may correspond to the direction away from the base surface 104 (e.g., starting from the center of the housing 102). Thus, when OBD 100-1 is positioned on the skin, the proximal direction may correspond to the direction towards the skin and the distal direction may correspond to the direction away from the skin. In FIG. 1B-1, the proximal direction is downwards, and the distal direction is upwards. The height of OBD 100-1 is in a direction parallel to the central axis A (vertical in FIG. 1B-1). The width of OBD 100-1 is in a direction perpendicular to the central axis A (horizonal in FIG. 1B-1).

OBD 100-1 also comprises an adhesive patch 106. The adhesive patch 106 may otherwise be referred to as an adhesive pad or an adhesive sticker. The adhesive patch 106 is attached to the housing 102. In this embodiment, the adhesive patch 106 is attached to the base surface 104. The adhesive patch 106 is configured to adhere to the skin of the user. In this manner, the adhesive patch 106 is configured to attach OBD 100-1 to the skin of the user. More specifically, the adhesive patch 106 has a first side which is attached to the base surface 104. Thus, the first side comprises an adhesive for securing to the housing 102 (e.g., to the base surface 104). The adhesive patch 106 also has a second side (e.g., an opposite side) for attachment to the skin. Thus, the second side comprises an adhesive for securing to the skin. In this manner, when OBD 100-1 is arranged on the skin of the user, the adhesive patch 106 is arranged between the housing 102 and the skin (e.g., between the base surface 104 and the skin). The adhesive patch 106 may comprise a carrier material with adhesive on opposite sides of the carrier material. For example, the adhesive patch 106 may comprise a double-sided adhesive. The adhesive patch 106 may comprise a multi-layer structure with a carrier layer and two adhesive layers on opposing sides. Other layers may also be provided, such as multiple carrier layers and multiple adhesive layers. The adhesive patch 106 may be woven or non-woven (e.g., a woven or non-woven carrier material). The adhesive patch 106 may be elastic or non-elastic. In some examples, the adhesive patch 106 may be perforated.

In other examples, the adhesive patch 106 may be attached to the housing 102 at locations other than the base surface 104. In some examples, the adhesive patch 106 may be arranged over the housing 102. In this case, the adhesive patch 106 may be referred to as an overpatch. For example, after positioning the housing 102 on the skin, the overpatch may be placed over the housing 102. For example, the overpatch may contact the top of the housing 102 and may attach to the skin around the housing 102 (e.g., around the base 104). In some examples, the housing 102 may include an adhesive patch 106 on the base surface 104 and may also have an overpatch to be applied over the housing 102 after positioning on the skin.

In other examples, an adhesive patch 106 may not be provided. OBD 100-1, as well as all other OBD embodiments described herein, may comprise other forms of attachment to the body 5 instead of (or in addition to) an adhesive patch 106. For example, OBD 100-1 may be directly attached to the skin using adhesive (e.g., without a patch having a carrier material). In other examples, OBD 100-1 may be retained on the skin by using a non-adhesive band or wrap.

In this embodiment, the adhesive patch 106 is in contact with the base surface 104. In particular, the adhesive patch 106 is directly attached to the base surface 104. In other examples, the adhesive patch is not directly attached to the base surface 104 (for example, the adhesive patch 106 may not be in contact with the base surface 104). For example, if another component is positioned on the base surface 104, then the adhesive patch 106 may be in contact with that component (e.g., attached to that component).

In this embodiment, the adhesive patch 106 extends beyond the periphery of the housing 102. For example, the adhesive patch 106 extends beyond the periphery of the base surface 104. The periphery of the base surface 104 may be the same location as a peripheral edge of the base surface 104 (or a perimeter of the base surface 104). Specifically, the adhesive patch 106 has a width that is greater than a width of the housing 102. Thus, the adhesive patch 106 is wider than the base surface 104. For example, the adhesive patch 106 has a dimension in a direction perpendicular to the central axis A that is greater than the dimension of the housing 102 in that direction. This increases the adhesive surface area, providing a more secure attachment. This also allows the user to press directly on the top of the adhesive patch 106 around the housing 102 to secure OBD 100-1 to the skin (e.g., a section of the adhesive patch 106 may be provided without adhesive on the top side). At the end of the wear duration, the section of the adhesive patch 106 protruding beyond the housing 102 also provides a tab for easier removal of OBD 100-1 by peeling the adhesive patch 106 from the skin. In other examples, the adhesive patch 106 may be the same size as the housing 102 or smaller. The adhesive patch 106 may have any shape defined by its outer perimeter edge. The adhesive patch 106 may have a similar shape to the shape of the base surface 104. For example, the adhesive patch 106 may extend beyond the periphery of the base surface 104 by a substantially constant distance around the periphery of the base surface 104. The adhesive patch 106 may have a shape that is different from that of the base surface 104. The adhesive patch 106 may have a shape similar to any of those described with respect to the shape of OBD 100 of FIGS. 1N-3 through 1N-20 or elsewhere.

In this embodiment, the base surface 104 is flat. In particular, the base surface 104 extends in a plane. This provides a good mechanical attachment surface for securely attaching the housing 102 to the skin by maximizing the surface area. In this manner, the base surface 104 lies on a plane parallel to the skin. Although the base surface 104 is generally flat, the base surface 104 may include various surface features, such as channels, recesses, or protrusions. In other examples, the base surface 104 is not flat, and may be convex or concave, for example. For example, the base surface 104 may be rounded (e.g., having convexity or concavity) or may be textured (e.g., with surface roughness), or may be multi-faceted. In some examples, the base of the housing 102 may include multiple surfaces, for example a further surface in addition to the base surface 104. The base surface 104 may be the surface configured for attachment to the adhesive patch 106 (e.g., the proximal-most surface) and the further surface may be recesses relative to the base surface 104 (e.g., positioned further in the distal direction).

In this embodiment, the housing 102 also comprises a top surface 108. The top surface 108 is a surface of the housing 102. In particular, the top surface 108 is an exterior surface of the housing 102 (e.g., an outer surface, or an external surface). The top surface 108 is arranged at the top of the housing 102. The housing 102 may comprise a top. The top may be a top portion of the housing 102. The top surface 108 may alternatively be referred to as an upper surface, a distal surface, a non-adhesive-contacting surface, or a non-skin-facing surface. The top surface 108 is configured to be positioned away from the skin of the user. Specifically, when OBD 100-1 is placed on the skin of the user, the top surface 108 faces away from the skin.

The proximal direction may correspond to the direction away from the top surface 108 (e.g., starting from the center of the housing 102 or from the base surface 104). The distal direction may correspond to the direction towards the top surface 108 (e.g., starting from the center of the housing 102 or from the base surface 104). The top surface 108 is arranged more distally than the base surface 104. For example, the top surface 108 is arranged further in the distal direction compared to the base surface 104. The top surface 108 may be the distal-most surface. In this embodiment, the top surface 108 is opposite the base surface 104. In other words, the top surface 108 is arranged at an opposite side of the housing 102 to the base surface 104.

In this embodiment, the top surface 108 is generally parallel to the base surface 104. Thus, the top surface 108 is flat. In particular, the top surface 108 extends in a plane. This contributes to the low profile of the housing 102. In this manner, the top surface 108 lies on a plane generally parallel to the skin. Although the top surface 108 is generally flat, the top surface 108 may include various surface features, such as channels, recesses, or protrusions. In other examples, the top surface 108 is not flat, and may be convex or concave, for example. For example, at least a portion of the top surface 108 may have a dome shape or an indented shape. For example, the top surface 108 may be rounded (e.g., having convexity or concavity) or may be textured (e.g., with surface roughness), or may be multi-faceted. In some examples, the top of the housing 102 may include multiple surfaces, for example a further surface in addition to the top surface 108. In this embodiment, the top surface 108 has a similar shape to the base surface 104. In other examples, the top surface 108 may have a different shape to the base surface 104. For example, the top surface 108 may not be parallel to the base surface 104.

In this embodiment, the housing 102 also comprises a side surface 110. The side surface 110 is a surface of the housing 102. In particular, the side surface 110 is an exterior surface of the housing 102 (e.g., an outer surface, or an external surface). The side surface 110 is arranged at the side (or periphery) of the housing 102. In this manner, the housing 102 has a base surface 104, a top surface 108, and a side surface 110. The shapes and/or contours of the base surface 104, top surface 108, and side surface 110, for this and all the other embodiments of housings described herein, are an aesthetic design choice. The shapes and/or contours of the edges or other boundaries between the base surface 104, top surface 108, and/or side surface 110, whether sharp, rounded, radiused, or otherwise, for this and all other embodiments of housings described herein, are also an aesthetic design choice. The side surface 110 may alternatively be referred to as an edge surface, a peripheral surface, or an upstanding surface. In some examples, the side surface 110 may be referred to as a side wall, an edge wall, a peripheral wall, or an upstanding wall.

In this embodiment, the side surface 110 is connected to the base surface 104. In particular, the side surface 110 is connected to a periphery of the base surface 104. In this embodiment, the side surface 110 is also connected to the top surface 108. In particular, the side surface 110 is connected to a periphery of the top surface 108. In this manner, the side surface 110 extends between the base surface 104 and the top surface 108, joining these surfaces together. Thus, the side surface 110 connects the base surface 104 to the top surface 108. In this manner, the top surface 108 does not contact the skin (or the adhesive patch 106).

The side surface 110 extends in a direction from the base surface 104 towards the top surface 108. In this embodiment, the side surface 110 extends in the distal direction. In particular, the side surface 110 extends entirely in the distal direction. The side surface 110 extends in a direction parallel to the central axis A. Thus, when OBD 100-1 is placed on the skin of the user, the side surface 110 extends from the base surface 104 in a direction away from the skin to the top surface 108. For example, the side surface 110 extends from the base surface 104 in a direction away from the adhesive patch 106. In this embodiment, the side surface 110 extends in a direction perpendicular to the base surface 104. The side surface 110 also extends in a direction perpendicular to the adhesive patch 106. Since, in this example, top surface 108 is parallel to base surface 104, the side surface 110 also extends in a direction perpendicular to the top surface 108. When the base surface 104 is arranged horizontally (and the top surface 104 consequently is also arranged horizontally), the side surface 110 extends substantially vertically (e.g., as shown in FIG. 1B-1).

In this embodiment, the base surface 104, the side surface 110, and the top surface 108 together define the exterior surfaces (otherwise referred to as outer surfaces or external surfaces) of the housing 102. In this embodiment, these surfaces together enclose the various components (such as electronics 122) within the housing 102. In some examples, this provides an interior space in which the components (such as electronics 122) can be arranged. The housing 102 may comprise interior surfaces (e.g., inner surfaces, or internal surfaces). The interior surfaces may be opposite to the exterior surfaces. For example, the housing 102 may comprise a structure (e.g., an enclosure, or one or more walls) having a thickness, with the exterior surface on one side and the interior surface on the opposite side. For example, the base surface 104 may be the exterior surface of a part of the structure and the structure may have an interior surface opposite the base surface 104. For example, the housing 102 may comprise a base wall, with the base surface 104 being an exterior surface of the base wall and an interior surface being on the opposite side of the base wall. The interior space may be hollow such that the components (e.g., electronics 122) partially fill the interior space. In other examples, the interior space is filled around the components, such as by molding or potting.

FIG. 1B-2 shows a perspective view of OBD 100-1 of FIG. 1B-1. This shows the three-dimensional shape of OBD 100-1. FIG. 1B-3 shows a bottom view of OBD 100-1 of FIG. 1B-1. In particular, the housing 102 is shown from below in FIG. 1B-3, with the adhesive patch 106 removed (but with patch 106's outer periphery represented in dashed line to illustrate its circular shape were it present) to depict the exposed base surface 104.

As shown, in this embodiment, the housing 102 is generally circular. More specifically, the base surface 104 is circular. The periphery of the base surface 104 therefore corresponds to a circumference of the base surface 104. In turn, a width of the base surface 104 (in a direction perpendicular to and intersecting the central axis A) corresponds to a diameter of the base surface 104. As shown, the top surface 108 is also circular. In this embodiment, the top surface 108 has the same diameter as the base surface 104. Thus, a diameter of the housing 102 is the same as the diameter of the base surface 104 and the diameter of the top surface 108. In this example, the adhesive patch 106 is also circular, but may have other shapes in other examples. In this example, a diameter of adhesive patch 106 is greater than the diameter of the housing 102.

Generally speaking, any componentry (e.g., sensor electronics, printed circuit board, sensor connector, etc.) within the housing 102 is generic as to the shape of housing 102 and can thus work with a wide variety of alternative shapes.

Thus, in other examples, the OBD 100, such as housing 102 and base surface 104, may have a different shape, such as those described with reference to FIGS. 1N-3 through 1N-20. For example, the base surface 104 may have a curved or rounded shape (such as an oval shape, an ellipse shape, or a stadium shape). In some examples, the base surface 104 may have a polygon shape (such as a square shape, an oblong shape, or a triangular shape). For example, a square base surface 204 is shown in FIGS. 1C-1 and 1C-2. In some examples, the base surface 104 may have a shape with a combination of straight and rounded edges (such as a rounded square or other rounded polygon, or a truncated circle). Thus, the base surface 104 may be an irregular shape. The top surface 108 may have similar shapes. For example, top surface 108 may have a similar shape to base surface 104. In other examples, the top surface 108 may have a different shape to base surface 104. The shape of housing 102 may include one or more notches, protrusions, or recesses around the periphery, for example to aid engagement with an inserter (e.g., as described with reference to inserter 60). The housing 102 can be any color.

The side surface 110 extends from the base surface 104 in the distal direction to the top surface 108, thereby spacing the top surface 108 from the base surface 104. Overall, the housing 102 is generally disc shaped. The diameter of the housing 102 is greater than its height. For example, the dimension in the direction perpendicular to the central axis A is greater than the dimension in the direction parallel to the central axis A. In other examples, the housing 102 may have a different shape.

In this embodiment, since the housing 102 is circular, the side surface 110 extends around the circumference of the housing 102. The side surface 110 may be referred to as a circumferential surface, a circumferential wall, a circumferential side surface, or a circumferential side wall. The side surface 110 may be referred to as a cylindrical surface. More generally (such as where the housing 102 is not circular), the side surface 110 may extend around the perimeter of the housing 102 (e.g., a perimeter of the base surface 104). In this example, side surface 110 is one continuous surface around the entire periphery of the housing 102. In other examples, housing 102 may comprise multiple surfaces connected around the periphery.

In this embodiment, the housing 102 comprises a sensor aperture 112. In particular, the sensor aperture 112 is in the base surface 104. Thus, the base surface 104 comprises the sensor aperture 112. The sensor aperture 112 is an opening in the base surface 104. Since the base surface 104 is flat, the sensor aperture 112 is an aperture in the flat plane corresponding to the base surface 104. The sensor aperture 112 may be an opening in the base (e.g., a base portion of the housing 102). In this embodiment, the sensor aperture 112 is a circular hole. In other examples, other shapes may be provided such as a square hole. The sensor aperture 112 is an opening that permits passage through the base surface 104. In other examples, the sensor aperture 112 may be referred to as a first aperture or a base surface aperture.

In this embodiment, the sensor aperture 112 is arranged in the center of the base surface 104. The central axis A passes through the sensor aperture 112. In other examples, the sensor aperture 112 is not in the center, and may be arranged off-center, such as towards one side of the base surface 104. For example, the sensor aperture 112 may be arranged at a location on the base surface 104 between the central axis A and the side surface 110 (e.g., see OBD 100-13 of FIG. 1N-1).

In this embodiment, OBD 100-1 also comprises an adhesive aperture 114. In particular, the adhesive aperture 114 is in the adhesive patch 106. Thus, the adhesive patch 106 comprises the adhesive aperture 114. The adhesive aperture 114 is an opening in the adhesive patch 106. In this embodiment, the adhesive aperture 114 is a circular hole. In other examples, other shapes may be provided such as a square hole.

In this embodiment, the adhesive aperture 114 is arranged in the center of the adhesive patch 106. The central axis A passes through the adhesive aperture 114. The adhesive aperture 114 is aligned with the sensor aperture 112 in the base surface 104 along the central axis A. In this example, the adhesive aperture 114 is the same size and shape as the sensor aperture 112. In other examples, the adhesive aperture 114 may have a different size (e.g., smaller or larger than the sensor aperture 112) and/or may have a different shape. In other examples, the adhesive aperture 114 is aligned with the sensor aperture 112 (in a direction parallel to the central axis A) but both the sensor aperture 112 and the adhesive aperture 114 are not aligned with the central axis A (e.g., off-center).

The adhesive aperture 114 extends through the thickness of the adhesive patch 106 (in the direction parallel to the central axis A). This forms an opening between the housing 102 and the skin, when OBD 100-1 is placed on the skin. This allows the sensor aperture 112 to be in fluid connection with the skin.

In the case of partially implantable sensor 161, OBD 100-1 also comprises sensor 161. In this embodiment, the analyte sensor 161 is a glucose sensor. In other examples, the analyte sensor 161 may be configured to detect other analytes, as described herein. The analyte sensor 161 comprises a first portion 118. The analyte sensor 161 also comprises a second portion 120. The first portion 118 is retained within the housing 102. The analyte sensor 161 is described in more detail with reference to FIGS. 1O-1 to 1P-3, 1Q-1, and 1Q-2. The analyte sensor 161 is not limited to these implementations, and other forms of analyte sensor may be provided in or with OBD 100-1 in other examples.

OBD 100-1 also comprises sensor electronics 122. The first portion 118 of the analyte sensor 161 is coupled to the sensor electronics 122, by direct physical and electrical contact and/or by indirect electrical coupling, such as through one or more intermediary conductors, such that the electrodes of sensor 161 are electrically coupled with, communicatively coupled with, or in electrical communication with, contacts of sensor electronics 122. The first portion 118 can provide electrical contact between the analyte sensor 161 and the sensor electronics 122. The first portion 118 may be referred to as a contact portion. The sensor electronics 122 are shown for the purposes of illustration, which may not be to scale, and may have different shapes and sizes in other examples. For example, the sensor electronics 122 may extend to a greater or lesser extent in the height and width directions. In one example, the sensor electronics 122 may substantially fill the housing 102. The sensor electronics 122 are described in more detail with reference to FIGS. 1R-1 and 1R-2. The sensor electronics 122 are not limited to this implementation, and other forms of sensor electronics may be provided in OBD 100-1 in other examples. Example implementations of the sensor electronics 122 are shown in FIGS. 1N-1 and 1N-2.

The second portion 120 is configured to extend below the base surface 104 of the housing 102. The second portion 120 of the analyte sensor 161 is configured to be positioned in the body 5 of the user. Specifically, the second portion 120 is configured to be positioned under the skin of the user. In other examples, the second portion 120 may be configured to be positioned in the skin, such as in the dermal layer. In this embodiment, the second portion 120 is configured to be positioned in contact with bodily fluid of the user, such as in interstitial fluid (or dermal fluid or blood). In particular, the second portion 120 comprises a sensing region configured to sample the analyte level of the user. The second portion 120 may comprise electrodes for monitoring the analyte level of the user. The second portion 120 may be referred to as an insertion portion or a sensing portion.

In this embodiment, the first portion 118 is arranged in a first orientation and the second portion 120 is arranged in a second orientation. In particular, the first portion 118 is arranged generally perpendicularly to the second portion 120. The first portion 118 is perpendicular to the central axis A. For example, the first portion 118 is parallel to the base surface 104 (e.g., parallel to the skin, when OBD 100-1 is placed on the skin). This allows the first portion 118 to be conveniently coupled to the sensor electronics 122. For example, the sensor electronics 122 may include a printed circuit board (PCB) which is arranged to extend in a direction perpendicular to the central axis A (e.g., parallel to the base surface 104). In this embodiment, the first portion 118 is coupled to the PCB (e.g., specifically to the top side of the PCB, but may be coupled to the underside in other examples). The second portion 120 is parallel to the central axis A (in this example, the second portion 120 is generally aligned along the central axis A). For example, the second portion 120 is perpendicular to the base surface 104 (e.g., perpendicular to the skin, when OBD 100-1 is placed on the skin). In other examples, the angle between the first portion 118 and the second portion 120 is between 90° and 180° (e.g., an obtuse angle), such as between 90° and 135°.

The analyte sensor 161 is configured to extend through the base surface 104. In particular, the analyte sensor 161 is configured to extend through the sensor aperture 112. The analyte sensor 161 is also configured to extend through the adhesive patch 106. In particular, the analyte sensor 161 is configured to extend through the adhesive aperture 114. More specifically, the analyte sensor 161 passes through the sensor aperture 112 so that the second portion 120 protrudes from the housing 102 for insertion into the body 5 of the user. This allows the second portion 120 to be inserted into the body 5 for monitoring the analyte, while the first portion 118 can be coupled to the sensor electronics 122. Accordingly, when OBD 100-1 is positioned on the skin, the second portion 120 of the analyte sensor 161 is inserted into the body 5, as shown in FIG. 1B-1.

In this embodiment, the housing 102 also comprises a sharp aperture 124. In particular, the sharp aperture 124 is in the top surface 108. Thus, the top surface 108 comprises the sharp aperture 124. The sharp aperture 124 is an opening in the top surface 108. Since the top surface 108 is flat, the sharp aperture 124 is an aperture in the flat plane corresponding to the top surface 108. The sharp aperture 124 may be an opening in the top (e.g., a top portion of the housing 102). In this embodiment, the sharp aperture is a circular hole. In other examples, other shapes may be provided such as a square hole. The sharp aperture 124 is an opening through the top surface 108. In other examples, the sharp aperture 124 may be referred to as a second aperture or a top surface aperture.

In this embodiment, the sharp aperture 124 is arranged in the center of the top surface 108. The central axis A passes through the sharp aperture 124. The sharp aperture 124 is aligned with the sensor aperture 112 in the base surface 104 along the central axis A. In other examples, the sharp aperture 124 is not in the center of the top surface 108, and may be arranged off-center, such as towards one side of the top surface 108. For example, the sharp aperture 124 may be arranged at a location on the top surface 108 between the central axis A and the side surface 110 (e.g., see OBD 100-13 of FIG. 1N-1). In this example, the sharp aperture 124 is the same size and shape as the sensor aperture 112. In other examples, the sharp aperture 124 may have a different size (e.g., smaller or larger than the sensor aperture 112) and/or may have a different shape. In other examples, the sharp aperture 124 is aligned with the sensor aperture 112 (in a direction parallel to the central axis A) but both the sensor aperture 112 and the sharp aperture 124 are not aligned with the central axis A (e.g., off-center).

In this embodiment, the housing 102 also comprises a sensor passageway 126. The sensor passageway 126 may be referred to as a sensor passage, a sensor conduit, a sensor duct, or a sensor lumen. The sensor passageway 126 extends between the sensor aperture 112 and the sharp aperture 124. In other words, the sensor aperture 112 is connected to the sharp aperture 124 by the sensor passageway 126. The sensor passageway 126 provides a passage through the housing 102 between the base surface 104 and the top surface 108. For example, the top surface 108 may be in fluid communication with the base surface 104 through the sensor passageway 126. In this embodiment, the sensor passageway 126 is cylindrical (e.g., having a circular cross section). In other examples, the sensor passageway 126 may have different shapes.

In this embodiment, the sensor passageway 126 is arranged along the central axis A. In other examples, the sensor passageway 126 is not in the center, and may be arranged off-center, such as towards one side of the housing 102 (e.g., see OBD 100-13 of FIG. 1N-1). In this embodiment, the sensor passageway 126 is straight and extends along the central axis A (e.g., perpendicular to the base surface 104). In other examples, the sensor aperture 112 is not aligned with the sharp aperture 124 (e.g., along the central axis A or a direction parallel to the central axis A). For example, the sensor passageway 126 may be angled (e.g., oblique).

In this embodiment, the sharp aperture 124 is configured to receive a sharp. For example, the sharp may be referred to as a needle. In particular, the sharp aperture 124 is configured to receive a sharp for insertion of the analyte sensor 161. A sharp can be inserted into the sharp aperture 124, passing along the sensor passageway 126, and through the sensor aperture 112. The sharp can then be inserted into the body 5.

The analyte sensor 161 extends along part of the sensor passageway 126. In particular, the analyte sensor 161 extends from the first portion 118 connected to the sensor electronics 122 and into the sensor passageway 126. The sensor passageway 126 may have an opening in a sensor passageway wall, where the opening receives the analyte sensor 161. The opening may be sealed around the analyte sensor 161 to prevent ingress of contaminants (such as moisture) into the sensor electronics 122.

The analyte sensor 161 may be positioned within the sharp. In particular, the analyte sensor 161 (in particular at least the second portion 120) may be positioned within an opening of the sharp. For example, the sharp may have an open cross section (such as a U-shape, a V-shape, or a C-shape), and the analyte sensor 161 can fit into the opening. In other examples, the analyte sensor 161 may be arranged adjacent to the sharp. The sharp can protrude from the sensor aperture 112 with the second portion 120 of the analyte sensor 161 for insertion into the body 5.

OBD 100-1 can then be moved towards the skin, and the sharp can be inserted into the skin, carrying the second portion 120 of the analyte sensor 161 into the body 5. For example, OBD 100-1 can be advanced towards the skin by an inserter (such as the inserter 60). The sharp can then be withdrawn from the skin, leaving the second portion 120 of the analyte sensor 161 in the body 5. The sharp can be withdrawn from OBD 100-1 through the sharp aperture 124.

In some examples, the second portion 120 of the analyte sensor 161 may be inserted into the skin at an angle relative to the central axis A. As a result, the sharp may be inserted at an angle relative to the central axis A (and thus relative to the skin). As a result, the sensor passageway 126 may be angled such that the sharp aperture 124 is offset from the sensor aperture 112 (e.g., in a direction perpendicular to the central axis A). For example, the sensor passageway 126 may have an oblique cylinder shape.

In this embodiment, the sensor passageway 126 passes through the sensor electronics 122. To allow this, the sensor electronics 122 may include an aperture for receiving the sensor passageway 126. For example, a PCB of the sensor electronics 122 may include an opening for the sensor passageway 126 to extend through. The sensor electronics 122 may then extend around the sensor passageway 126.

In other examples, the sensor passageway 126 may have other shapes and sizes (such as a changing width, e.g., a tapered or stepped configuration). In other examples, the sensor aperture 112 and the sharp aperture 124 may have similar or different shapes and sizes to each other.

In other examples, the sharp aperture 124 is not provided. The sensor passageway 126 may terminate in the housing 102 after the position where the analyte sensor 161 passes through the sensor passageway wall for connection to the sensor electronics 122. For example, the analyte sensor 161 may be inserted without a sharp. In one example, the tip of the second portion 120 of the analyte sensor 161 may be sharp to allow insertion into the body 5 without use of a separate sharp. In other examples, a sharp may be provided as part of OBD 100-1, and for example may be retracted within the housing 102 after inserting sensor 161.

In some examples, the housing 102 comprises a proximal side 128. The housing 102 may also comprise a distal side 130. The proximal side 128 may be referred to as a first side, a bottom side, a lower side, an adhesive-contacting side, or a skin-facing side. In this embodiment, the base surface 104 is arranged at the proximal side 128. The top surface 108 is arranged away from the proximal side 128. The distal side 130 may be referred to as a second side, a top side, an upper side, a non-adhesive-contacting side, or a non-skin-facing side. In this embodiment, the top surface 108 is arranged at the distal side 130. The base surface 104 is arranged away from the distal side 130.

In some examples, the housing 102 comprises a proximal region and a distal region. The proximal region may refer to a region of the housing 102 towards the proximal side 128. For example, the base (e.g., the base portion) may be the proximal region. The base surface 104 may be on the proximal region. The distal region may refer to a region of the housing 102 towards the distal side 130. The top (e.g., the top portion) may be the distal region. The top surface 108 may be on the distal region. In some examples, a midpoint of the housing 102 may extend through a center of the housing 102 in a plane extending in a direction perpendicular to the central axis A. The midpoint may be half-way between the proximal side 128 and the distal side 130 (e.g., half-way between the base surface 104 and the top surface 108). The proximal region may refer to a region of the housing 102 on the proximal side of the midpoint and the distal region may refer to a region of the housing 102 on the distal side of the midpoint.

It is desirable for devices inserted in the body to be sterile for insertion. Sterilization can include any number of processes that reduce (e.g., effectively eliminate or kill) pathogens, such as bacteria, fungi, and viruses. These pathogens, if not eliminated from the device, may cause health problems to the user. Accordingly, the analyte sensor 161 may be sterilized prior to insertion (e.g., during manufacture). This may be performed by any known technique. For example, the analyte sensor 161 may be sterilized using radiation (e.g., electron beam or “e-beam” irradiation, or low-energy X-ray irradiation). Radiation sterilization may be delivered through continuous or pulsed irradiation focused on a target location. In some examples, radiation sterilization may damage electronics components, so it may be desirable to sterilize the analyte sensor 161 separately. For example, other components such as the analyte sensor 161 and the sharp may be sterilized separately or while shielding the sensor electronics 122 from the radiation.

In other examples, the sterilization may comprise gas sterilization (otherwise referred to as chemical sterilization). For example, the sterilization may comprise ethylene oxide or “EO” sterilization. This may allow the components to be sterilized without damaging the sensor electronics 122. Combinations of the above sterilization techniques may also be used, for example to sterilize different parts of the device.

OBD 100-1 is shown in a state after assembly and insertion into the body. OBD 100-1 here, and in the other embodiments herein, can be assembled and inserted into the body in a variety of different ways. Examples of insertion and assembly techniques for partially implantable sensors 161 are shown and described with respect to FIGS. 1S-1, 1S-2, 1T-1 through 1T-6, 1U-1 through 1U-22, and all OBDs 100 with partially implantable sensors 161 are configurable for operation with respect to these insertion and assembly techniques.

In other examples, if OBD 100-1 is assembled in a controlled environment, there may be no need to sterilize the sensor electronics 122. Instead, coupling of components (e.g., mechanical coupling or adhesive coupling) may provide a sufficiently sterile barrier for the assembled housing 102, preventing contaminants from reaching the analyte sensor 161 until use. For example, a sensor cap may be located over the sterilized analyte sensor 161 and coupled to the base surface 104 of the housing 102, forming a sterile barrier. The sensor cap can then be removed before use, such as by the inserter, for example when removing a cap of the inserter, such as in the embodiment described with respect to FIGS. 1U-14 and 1U-15.

In some examples, the housing 102 may be formed by various manufacturing techniques, such as stamping, casting, injection molding (e.g., one-shot molding), sintering, two-shot molding (or multi-shot or over-molding), or any combination thereof, especially depending on the number of separate components. For example, the housing 102 may be molded from a rigid material such as plastic (e.g., polycarbonate or ABS). In other examples, the material may be metal, composite (e.g., fiberglass-reinforced laminate, such as FR4), or other polymer (e.g., polyimide). In some examples, the housing 102 may include a piece with an over-molded component such as a seal (e.g., formed of an elastomer, rubber, a polymer, or other pliable material). In some examples, one or more pieces of the housing 202 may be formed of a clear or transparent material (such as the base surface 104), such as a clear plastic (e.g., polycarbonate or ABS). In some examples, the housing 102 may be formed from different materials.

Turning now to embodiments with a wholly (or fully) implantable sensor, FIG. 1B-4 is a partial cross-sectional view depicting an example embodiment of OBD 100-1′ with a wholly implanted sensor 162 in wireless communication with OBD 100-1′ over communication path 115. OBD 100-1′ and sensor 162 can communicate using one or more wireless communication protocols that may be the same or different as those used by OBD-100 and/or OBD 100-1′ to communicate with receiver units 20. Sensor 162 is wholly implanted within body 5 of the subject such that no physical connection exists with OBD 100-1′. Sensor 162 can have its own power source within it or can rely on power harvesting from the wireless signals transmitted to it by OBD 100-1′, or both. The subject can visit a medical professional and undergo an implantation procedure (e.g., inpatient or outpatient operation or surgery) where an incision is created in the subject's body 5 and sensor 162 is placed at a location wholly within the subject's body. As described further herein, the operation can be performed manually or with a specialized implantation tool. This implant location can be, in whole or in part, within the epidermis, within the dermis, within the subcutaneous layer, or elsewhere provided sensor 162 has access to the analyte being measured and can communicate wirelessly with OBD 100-1′. The incision is then closed (e.g., with a suture, staple, or adhesive) so that it may heal.

In this embodiment, wholly implanted sensor 162 wirelessly communicates the glucose level, or other analyte level, to OBD 100-1′ which remains outside of the body on the skin in a position within wireless communication range of sensor 162. Sensor 162 is thus not maintained in physical and electrical contact with OBD 100-1′ while monitoring the analyte level. As such, embodiments of OBD 100-1′ operating with a wholly implantable sensor 162 can omit the adhesive aperture, sensor aperture, sensor passageway, and sharp aperture, as can be seen by a comparison of FIGS. 1B-1 and 1B-4, as those features are not necessary if a partially implantable sensor is not present. Thus, all of the embodiments of OBD 100 described herein as having an adhesive aperture, sensor aperture, sensor passageway, and sharp aperture to accommodate a partially implantable sensor 161, can likewise be configured without an adhesive aperture, sensor aperture, sensor passageway, and sharp aperture to operate with a wholly implantable sensor 162 or a wholly ex vivo sensor.

An example embodiment of sensor electronics for interfacing with a wholly implantable sensor 162 is described with respect to FIG. 1R-2. OBD 100-1′ configured to operate with a wholly implantable sensor 162 can be a small, single-use (e.g., non-rechargeable) or reusable (e.g., rechargeable) device with a designed wear duration as described herein (e.g., one day, 14 days, 15 days, 20 days, 21 days, 28 days, 30 days, 60 days, etc.). The sensor life of wholly implanted sensor 162 can be considerably longer than that of sensor 161.

Such a sensor 162 can operate using the electrochemical sensing configurations, techniques, and variations described herein with respect to sensor 161. Sensor 162 can alternatively sense a glucose and/or other analyte level through a photonic-based sensing technique and data indicative of the glucose and/or other analyte level can be wirelessly communicated from sensor 162 to OBD 100-1′ through the body 5. Various photonic-based sensing techniques can be used. For example, an indicator chemical in sensor 162 can bond or react in the presence of glucose and/or another analyte and then fluoresce in proportion to the glucose and/or the other analyte level in response to received light. The amount of fluorescence can be measured with a detector and the level can be determined based on the measured fluorescence. Sensor 162 can have multiple sensing regions for sensing different analytes. These sensing regions can each utilize their own discrete sensing componentry. Alternatively, as with sensors 161A and 161B, the multiple sensing regions of sensor 162 can share sensing componentry, such as electrodes or optical indicators, and also have discrete different sensing componentry for sensing the different analytes.

In still other embodiments, sensor 160 may sense the glucose level and/or other analyte level while remaining wholly ex vivo, without implantation (neither partial nor full implantation). In these embodiments the sensor 160 and OBD 100 are physically and electrically coupled together, by the manufacturer or a user, and can be placed on the skin surface 10 but remain outside of the subject's body 5, e.g., the ex vivo sensor can be held adjacent to the body by OBD 100, either in contact with the skin surface or spaced apart from the skin but adjacent thereto. The wholly ex vivo sensor is five millimeters or less from the skin in certain embodiments. The wholly ex vivo sensor is ten millimeters or less from the skin surface in certain embodiments. OBD 100 with wholly ex vivo sensors can have a designed wear duration as described herein (e.g., one day, 10 days, 14 days, 15 days, 20 days, 21 days, 28 days, 30 days, or longer, etc.).

Such ex vivo sensors operate in the embodiments herein by utilizing a sensing architecture (technique and hardware). Such ex vivo architectures include, but are not limited to: electrochemical architectures such as Reverse Iontophoresis (RI) Technology; optical architectures such as Fluorescence Methods, Near-Infrared (NIR) and Mid-Infrared (MIR) Spectroscopy, Raman Spectroscopy, Optical Coherence Tomography (OCT), or Optical Polarimetry (OP); and microwave architectures utilizing parameters such as the reflection coefficient, resonance frequency, bandwidth, and quality factor (Q). Wholly ex vivo sensors can also be configured to detect and measure the concentration levels of two or more different analytes, such as any of those described herein.

Embodiments of OBD 100 operating in conjunction with an ex vivo sensor are different from, and do not include, in vitro devices that rely on penetration of the subject's body 5 (e.g., with a sharp or conduit) to remove bodily fluid from body 5 and test the bodily fluid while outside of the subject's body to determine the analyte level. One example of an in vitro device is a blood glucose meter where the subject performs a fingerstick to place blood on a test strip that is then inserted into the meter for glucose level measurement.

In embodiments of system 10 including a partially implantable sensor 161, OBD 100 operates in conjunction with that sensor 161 through a physical connection and electrical contact between the partially implantable sensor 161 and OBD electronics 122. Portion 118 of the sensor 161 can be contained within housing 104. When connected together OBD 100 can be considered to include sensor 161. In embodiments of system 10 including a wholly implantable sensor 162, OBD 100 operates in conjunction with that sensor 162 through communication over the wireless communication path 115 between OBD 100 and sensor 162. Sensor 162 is included within system 10 but is not included within OBD 100 as the two are spaced apart in operation and not connected physically nor via direct electrical contact. In embodiments of system 10 including a wholly ex vivo sensor, OBD 100 operates in conjunction with that sensor through a physical connection and one or more signal connections (e.g., electrical, photonic) between the wholly ex vivo sensor and OBD electronics 122. A portion of the wholly ex vivo sensor can be contained within housing 104. When connected together OBD 100 can be considered to include the wholly ex vivo sensor.

### On-Body Device 100-2

Referring to FIGS. 1C-1 and 1C-2, an OBD 100-2 is shown according to an embodiment. OBD 100-2 may be the same as OBD 100-1 of FIGS. 1B-1 to 1B-3, and may include the same features, except as explained below. The optional features and alternatives described in relation to OBD 100-1 may similarly be applied to OBD 100-2 in a corresponding manner.

In this embodiment, OBD 100-2 is the same as OBD 100-1, except that OBD 100-2 has a housing 202 with a different shape compared to the housing 102. In this embodiment, the housing 202 comprises a base surface 204 having a square shape, in contrast to the circular shape of the base surface 104 of OBD 100-1. The base surface 204 comprises four edges, each defining a section of the periphery of the base surface 204. The width of the base surface 204 refers to the dimension between opposing sides of the square. The base surface 204 is otherwise similar to the base surface 104.

In FIGS. 1C-1 and 1C-2, OBD 100-2 is shown with the adhesive patch removed (for the purposes of illustration) to show the exposed base surface 204. In some examples, OBD 100-2 comprises an adhesive patch similar to the adhesive patch 106 (e.g., a circular shape). In other examples, the adhesive patch may have other shapes.

In this embodiment, the housing 202 also comprises a top surface 208 having a square shape. The top surface 208 has the same shape and size as the base surface 204. In other examples, the top surface 208 may have another shape, or a different size, to the base surface 204. The top surface 208 is otherwise similar to the top surface 108.

In this embodiment, the housing 202 also comprises a side surface 210. The side surface 210 comprises a plurality of sections. In this case, the side surface 210 has four sections. Each section joins a particular section of the periphery of the base surface 204 (e.g., one edge of the square) to a particular section of the periphery of the top surface 208 (e.g., one edge of the square). Thus, each section has a rectangular shape. Each section of the side surface 210 is connected to an adjacent section, and the sections are arranged around the periphery (e.g. separated by corners of the housing 202). Each section of the side surface 210 may be referred to as a face. In this embodiment, the sections of the side surface 210 are flat. In this embodiment, the side surface 210 is perpendicular to the base surface 204. The side surface 210 is otherwise similar to the side surface 110. Overall, the housing 202 is cuboid.

Although the square shape of the housing 102 is shown with sharp corners, in some examples the corners may be rounded.

OBD 100-2 comprises an analyte sensor 161 and sensor electronics 122 (not shown) in a similar manner to OBD 100-1. In other examples, variations to the analyte sensor 161 and/or the sensor electronics 122 described herein may be applied to OBD 100-2.

OBD 100-2 is shown and described with a partially implantable sensor but can be configured to operate with a wholly implantable sensor or wholly ex vivo sensor, in which case the adhesive aperture, sensor aperture, sensor passageway, and sharp aperture can be omitted.

### On-Body Device 100-3

Referring to FIG. 1D, an OBD 100-3 is shown according to an embodiment. OBD 100-3 may be the same as OBD 100-1 of FIGS. 1B-1 to 1B-3, and may include the same features, except as explained below. The optional features and alternatives described in relation to OBD 100-1 may similarly be applied to OBD 100-3 in a corresponding manner. In some examples, OBD 100-3 may be similar to OBD 100-2 of FIGS. 1C-1 and 1C-2 and features described in relation to those embodiments may be applied to OBD 100-3.

In this embodiment, OBD 100-3 is the same as OBD 100-1, except that OBD 100-3 has a housing 302 with a different shape compared to the housing 102. The housing 302 comprises a base surface 304. The adhesive patch 106 is attached to the base surface 304 in a similar manner to OBD 100-1. In this embodiment, the housing 302 comprises a side surface 310 that extends inwards (e.g., towards an interior region of the housing 102, e.g., towards the central axis A), in contrast to the perpendicular side surface 110. In particular, the side surface 310 extends in a direction that has a component in the distal direction and a component towards the central axis A. In other examples, the side surface 310 may extend outwards. The housing 302 comprises a top surface 308 which has a smaller size than the base surface 304. The base surface 304 and the top surface 308 are circular and parallel as in OBD 100-1.

In this embodiment, the side surface 310 is angled. In particular, the side surface 310 is arranged to extend at an angle to the central axis A. The side surface 310 is angled inwards (e.g., radially inwards towards the central axis A) as it extends from the base surface 304 towards the top surface 308. In this manner, the housing 302 has a truncated triangle shape in cross section (e.g., as shown in FIG. 1D). This may be referred to as a trapezium shape (e.g., a trapezoid shape). The side surface 310 is straight in cross section (e.g., as shown in FIG. 1D).

In this example, since the base surface 304 and the top surface 308 are circular, the housing 302 has a truncated conical shape. This may be referred to as a frustoconical shape. The side surface 310 forms a partial cone shape. In particular, the side surface 310 forms a cone shape from the base surface 304 until it meets the top surface 308, which forms a flattened top to the partial cone. In this manner, the side surface 310 forms a conical surface or a conical wall (in contrast to the cylindrical surface of OBD 100-1).

OBD 100-3 comprises an analyte sensor 161 and sensor electronics 122 in a similar manner to OBD 100-1. In other examples, variations to the analyte sensor 161 and/or the sensor electronics 122 described herein may be applied to OBD 100-3.

OBD 100-3 is shown and described with a partially implantable sensor but can be configured to operate with a wholly implantable sensor or wholly ex vivo sensor, in which case the adhesive aperture, sensor aperture, sensor passageway, and sharp aperture can be omitted.

In other examples, the base surface 304 and the top surface 308 may have different shapes, such as the square shape shown in OBD 100-2, or the other shapes described with reference to OBD 100. The angled side surface 310 may be applied to different shapes, such as the square shape or other shape. Thus, for a square shape, the side surface 310 may include a plurality of sections, such as different sections connected to different edges of the square. In that case, the housing 302 may have a truncated pyramid shape. This may be referred to as a frustum shape (e.g., a square frustum) or frusto-pyramidal shape. The side surface 310 forms a partial pyramid shape from the base surface 304 until it meets the top surface 308, which forms a flattened top to the partial pyramid.

### On-Body Device 100-4

Referring to FIG. 1E, an OBD 100-4 is shown according to an embodiment. OBD 100-4 may be the same as OBD 100-1 of FIGS. 1B-1 to 1B-3, and may include the same features, except as explained below. The optional features and alternatives described in relation to OBD 100-1 may similarly be applied to OBD 100-4 in a corresponding manner. In some examples, OBD 100-4 may be similar to OBD 100-2 of FIGS. 1C-1 and 1C-2, or OBD 100-3 of FIG. 1D, and features described in relation to those embodiments may be applied to OBD 100-4.

In this embodiment, OBD 100-4 is the same as OBD 100-1, except that OBD 100-4 has a housing 402 with a different shape compared to the housing 102. The housing 402 comprises a base surface 404. The adhesive patch 106 is attached to the base surface 404 in a similar manner to OBD 100-1. In this embodiment, the housing 402 comprises a side surface 410 that extends inwards (e.g., towards the central axis A), in contrast to the perpendicular side surface 110, and in a similar manner to OBD 100-3. In other examples, the side surface 410 may extend outwards. The housing 402 comprises a top surface 408 which has a smaller size than the base surface 404. The base surface 404 and the top surface 408 are circular and parallel as in OBD 100-1. In contrast to the angled side surface 310 of OBD 100-3, OBD 100-4 comprises a side surface 410 that is curved. The side surface 410 is curved inwards (e.g., radially inwards towards the central axis A) as it extends from the base surface 404 to the top surface 408. In this manner, the housing 402 has a truncated dome shape in cross section (e.g., as shown in FIG. 1E). The side surface 410 is curved in cross section (e.g., as shown in FIG. 1E).

The side surface 410 extends in a curve partially in the distal direction and partially towards the central axis A. The radius of curvature may be constant, or it may vary over the extent of the side surface 410 from the base surface 404 to the top surface 408.

In this example, since the base surface 404 and the top surface 408 are circular, the housing 402 has a truncated dome shape. The side surface 410 forms a partial dome shape. In particular, the side surface 410 forms a dome shape from the base surface 404 until it meets the top surface 408, which forms a flattened top to the partial dome. The side surface 410 forms a dome surface or a dome wall (in contrast to the cylindrical surface of OBD 100-1).

OBD 100-4 comprises an analyte sensor 161 and sensor electronics 122 in a similar manner to OBD 100-1. In other examples, variations to the analyte sensor 161 and/or the sensor electronics 122 described herein may be applied to OBD 100-4.

OBD 100-4 is shown and described with a partially implantable sensor but can be configured to operate with a wholly implantable sensor or wholly ex vivo sensor, in which case the adhesive aperture, sensor aperture, sensor passageway, and sharp aperture can be omitted.

In other examples, the base surface 404 and the top surface 408 may have different shapes, such as the square shape shown in OBD 100-2, or the other shapes described with reference to OBD 100. The curved side surface 410 may be applied to different shapes, such as the square shape or other shapes.

### On-Body Device 100-5

Referring to FIG. 1F, an OBD 100-5 is shown according to an embodiment. OBD 100-5 may be the same as OBD 100-1 of FIGS. 1B-1 to 1B-3, and may include the same features, except as explained below. The optional features and alternatives described in relation to OBD 100-1 may similarly be applied to OBD 100-5 in a corresponding manner. In some examples, OBD 100-5 may be similar to OBD 100-2 of FIGS. 1C-1 and 1C-2, OBD 100-3 of FIG. 1D, or OBD 100-4 of FIG. 1E, and features described in relation to those embodiments may be applied to OBD 100-5.

In this embodiment, OBD 100-5 is the same as OBD 100-1, except that OBD 100-5 has a housing 502 with a different shape compared to the housing 102. The housing 502 comprises a base surface 504. The adhesive patch 106 is attached to the base surface 504 in a similar manner to OBD 100-1. In this embodiment, the housing 502 comprises a top surface 508. However, in contrast to the housing 102, the housing 502 does not have a separate side surface (such as side surface 110). Instead, the top surface 508 replaces the combination of the top surface 108 and the side surface 110.

In this embodiment, the top surface 508 is connected to the base surface 504. In particular, the top surface 508 is connected to the periphery of the base surface 504. The top surface 508 is connected around the periphery of the base surface 504. In this embodiment, the base surface 504 is circular, and the top surface 508 extends around the circumference of the base surface 504.

In this embodiment, the base surface 504 and the top surface 508 together define the exterior surfaces (otherwise referred to as outer surfaces or external surfaces) of the housing 502. In this embodiment, the various components (such as the sensor electronics 122) are enclosed within the housing 502 between the base surface 504 and the top surface 508.

The top surface 508 extends in a curve partially in the distal direction and partially inwardly (e.g., towards the central axis A). In this embodiment, the top surface 508 is curved.

In this embodiment, the housing 502 has a dome shape. This is similar to OBD 100-4, except that the housing 502 is a full dome shape instead of a truncated dome shape, since there is no flattened top. The radius of curvature may be constant, or it may vary over the extent of the top surface 508 from the base surface 504 to the central axis A. For example, the top surface 508 may define a hemisphere, or a steeper or shallower dome.

Instead of the curved top surface 508, the top surface 508 may be angled. For example, the housing 502 may have a conical shape. The top surface 508 may be angled, connected around the periphery of the circular base surface 504, thereby forming a cone shape. This is similar to that described in relation to OBD 100-3, except that the housing 502 would be a full conical shape instead of a truncated conical shape, since there is no flattened top.

OBD 100-5 comprises an analyte sensor 161 and sensor electronics 122 in a similar manner to OBD 100-1. In other examples, variations to the analyte sensor 161 and/or the sensor electronics 122 described herein may be applied to OBD 100-5.

OBD 100-5 is shown and described with a partially implantable sensor but can be configured to operate with a wholly implantable sensor or wholly ex vivo sensor, in which case the adhesive aperture, sensor aperture, sensor passageway, and sharp aperture can be omitted.

In other examples, the base surface 504 may have different shapes, such as the square shape shown in OBD 100-2, or the other shapes described with reference to OBD 100. The top surface 508 may be applied to different shapes, such as the square shape or other shape. Thus, for a square shape, the top surface 508 may include a plurality of sections, such as different sections connected to different edges of the square. The top surface 508 may be angled, with each section connected to a different edge of the periphery of the square, thereby forming a pyramid shape. This is similar to that described in relation to OBD 100-3, except that the housing 502 would be a full pyramid shape instead of a truncated pyramidal shape, since there is no flattened top.

### On-Body Device 100-6

Referring to FIG. 1G, an OBD 100-6 is shown according to an embodiment. OBD 100-6 may be the same as OBD 100-1 of FIGS. 1B-1 to 1B-3, and may include the same features, except as explained below. The optional features and alternatives described in relation to OBD 100-1 may similarly be applied to OBD 100-6 in a corresponding manner. In some examples, OBD 100-6 may be similar to OBD 100-2 of FIGS. 1C-1 and 1C-2, OBD 100-3 of FIG. 1D, OBD 100-4 of FIG. 1E, or OBD 100-5 of FIG. 1F, and features described in relation to those embodiments may be applied to OBD 100-6.

In this embodiment, OBD 100-6 is the same as OBD 100-1, except that OBD 100-6 has a housing 602 with a different shape compared to the housing 102. The housing 602 comprises a base surface 604. The adhesive patch 106 is attached to the base surface 604 in a similar manner to OBD 100-1. The housing 602 also comprises a top surface 608 which has a smaller size than the base surface 604. The base surface 604 and the top surface 608 are circular and parallel as in OBD 100-1. In other examples, the base surface 604 and/or the top surface 608 are a different shape (e.g., square), with the top surface 608 smaller than the base surface 604. The housing 602 comprises a side surface 610 that extends circumferentially, as in OBD 100-1. In contrast to the housing 102, the housing 602 also comprises an intermediate surface 632.

In this embodiment, the intermediate surface 632 is arranged between the side surface 610 and the top surface 608. The intermediate surface 632 is a surface of the housing 602. In particular, the intermediate surface 632 is an exterior surface of the housing 602 (e.g., an outer surface, or an external surface). In this manner, the housing 602 has a base surface 604, a top surface 608, a side surface 610, and an intermediate surface 632.

The intermediate surface 632 is connected to the side surface 610. In particular, the intermediate surface 632 is connected to a periphery of the side surface 610. In this embodiment, the intermediate surface 632 is also connected to the top surface 608. In particular, the intermediate surface 632 is connected to a periphery of the top surface 608. In this manner, the intermediate surface 632 extends between the side surface 610 and the top surface 608, joining these surfaces together. Thus, the intermediate surface 632 connects the side surface 610 to the top surface 608.

In other examples, the intermediate surface 632 may be arranged between the base surface 604 and the side surface 610. In this manner, the intermediate surface 632 may connect the base surface 604 to the side surface 610. In some examples, the housing 602 comprises multiple intermediate surfaces 632. For example, the housing 602 may comprise a first intermediate surface 632 arranged between the side surface 610 and the top surface 608 and a second intermediate surface 632 arranged between the base surface 604 and the side surface 610.

In this embodiment, the intermediate surface 632 extends inwards (e.g., towards the central axis A). In particular, the intermediate surface 632 extends in a direction with a component in the distal direction and a component towards the central axis A. In other examples, the intermediate surface 632 may extend outwards.

In this embodiment, intermediate surface 632 is angled. In particular, the intermediate surface 632 is arranged to extend at an angle to the central axis A. In this manner, the intermediate surface 632 may be angled in a similar manner to the side surface 310 of OBD 100-3. The intermediate surface 632 is angled inwards (e.g., radially inwards towards the central axis A) as it extends from the side surface 610 towards the top surface 608. In this embodiment, the intermediate surface 632 provides a chamfered edge. For example, the housing 602 comprises a chamfered disc shape.

OBD 100-6 comprises an analyte sensor 161 and sensor electronics 122 in a similar manner to OBD 100-1. In other examples, variations to the analyte sensor 161 and/or the sensor electronics 122 described herein may be applied to OBD 100-6.

OBD 100-6 is shown and described with a partially implantable sensor but can be configured to operate with a wholly implantable sensor or wholly ex vivo sensor, in which case the adhesive aperture, sensor aperture, sensor passageway, and sharp aperture can be omitted.

In other examples, the base surface 604 and the top surface 608 may have different shapes, such as the square shape shown in OBD 100-2, or the other shapes described with reference to OBD 100. The angled intermediate surface 632 may be applied to different shapes, such as the square shape or other shape. Thus, for a square shape, the intermediate surface 632 may include a plurality of sections, such as different sections connected to different edges of the square. In that case, the housing 602 has a chamfered cuboid shape. Generally, the housing 602 may be a chamfered shape.

In other examples, the side surface 610 may not be perpendicular to the base surface 604. For example, the side surface 610 may be angled, such as in OBD 100-3, or curved, such as in OBD 100-4. For example, the side surface 610 may be angled and have a different angle to the intermediate surface 632.

### On-Body Device 100-7

Referring to FIG. 1H, an OBD 100-7 is shown according to an embodiment. OBD 100-7 may be the same as OBD 100-1 of FIGS. 1B-1 to 1B-3, and may include the same features, except as explained below. The optional features and alternatives described in relation to OBD 100-1 may similarly be applied to OBD 100-7 in a corresponding manner. In some examples, OBD 100-7 may be similar to OBD 100-2 of FIGS. 1C-1 and 1C-2, OBD 100-3 of FIG. 1D, OBD 100-4 of FIG. 1E, OBD 100-5 of FIG. 1F, or OBD 100-6 of FIG. 1G, and features described in relation to those embodiments may be applied to OBD 100-7.

In this embodiment, OBD 100-7 is the same as OBD 100-1, except that OBD 100-7 has a housing 702 with a different shape compared to the housing 102. The housing 702 comprises a base surface 704. The adhesive patch 106 is attached to the base surface 704 in a similar manner to OBD 100-1. The housing 702 also comprises a top surface 708 which has a smaller size than the base surface 704. The base surface 704 and the top surface 708 are circular and parallel as in OBD 100-1. In other examples, the base surface 704 and/or the top surface 708 are a different shape (e.g., square), with the top surface 708 smaller than the base surface 704. The housing 702 comprises a side surface 710 that extends circumferentially, as in OBD 100-1. In contrast to the housing 102, the housing 702 also comprises an intermediate surface 732, in a similar manner to OBD 100-6.

In contrast to the angled intermediate surface 632 of OBD 100-6, OBD 100-7 comprises an intermediate surface 732 that is curved. In this manner, the intermediate surface 732 may be curved in a similar manner to the side surface 410 of OBD 100-4. In particular, the intermediate surface 732 is curved inwards (e.g., radially inwards towards the central axis A) as it extends from the side surface 710 to the top surface 708. In this embodiment, the intermediate surface 732 provides a filleted edge. For example, the housing 702 comprises a filleted disc shape. For example, this may be similar to the shape of FIG. 1N-1.

The intermediate surface 732 extends in a curve partially in the distal direction and partially towards the central axis A. The radius of curvature may be constant, or it may vary over the extent of the intermediate surface 732 from the side surface 710 to the top surface 708.

In some examples, the curved intermediate surface 732 forms a smooth transition between the side surface 710 and the top surface 708. As a result, in some examples, the curved intermediate surface 732 may not be considered as a separate surface, but instead the side surface 710 smoothly transitions to the top surface 708 by a curved edge (or a radiused edge).

OBD 100-7 comprises an analyte sensor 161 and sensor electronics 122 in a similar manner to OBD 100-1. In other examples, variations to the analyte sensor 161 and/or the sensor electronics 122 described herein may be applied to OBD 100-7.

OBD 100-7 is shown and described with a partially implantable sensor but can be configured to operate with a wholly implantable sensor or wholly ex vivo sensor, in which case the adhesive aperture, sensor aperture, sensor passageway, and sharp aperture can be omitted.

In other examples, the base surface 704 and the top surface 708 may have different shapes, such as the square shape shown in OBD 100-2, or the other shapes described with reference to OBD 100. The curved intermediate surface 732 may be applied to different shapes, such as the square shape or other shape. Thus, for a square shape, the intermediate surface 732 may include a plurality of sections, such as different sections connected to different edges of the square. In that case, the housing 702 has a filleted cuboid shape. Generally, the housing 702 may be a filleted shape.

In other examples, the side surface 710 may not be perpendicular to the base surface 704. For example, the side surface 710 may be angled, such as in OBD 100-3, or curved, such as in OBD 100-4. For example, the side surface 710 may be curved and have a different radius of curvature to the intermediate surface 732.

### On-Body Device 100-8

Referring to FIG. 1I, an OBD 100-8 is shown according to an embodiment. OBD 100-8 may be the same as OBD 100-1 of FIGS. 1B-1 to 1B-3, and may include the same features, except as explained below. The optional features and alternatives described in relation to OBD 100-1 may similarly be applied to OBD 100-8 in a corresponding manner. In some examples, OBD 100-8 may be similar to OBD 100-2 of FIGS. 1C-1 and 1C-2, OBD 100-3 of FIG. 1D, OBD 100-4 of FIG. 1E, OBD 100-5 of FIG. 1F, OBD 100-6 of FIG. 1G, or OBD 100-7 of FIG. 1H, and features described for those embodiments may be applied to OBD 100-8.

In this embodiment, OBD 100-8 is the same as OBD 100-1, except that OBD 100-8 has a housing 802 with a different configuration compared to the housing 102. In particular, the housing 802 comprises a first part 834 and a second part 836. During manufacture, the first part 834 and the second part 836 are assembled together, with the sensor electronics 122 and the analyte sensor 161 arranged between.

In this embodiment, the first part 834 comprises the base surface 804. The first part 834 may be referred to as a base surface part, a bottom part, a lower part, a proximal part, an adhesive-contacting part, or a skin-facing part. For example, the first part 834 may correspond to a base (or base portion) of the housing 802. For example, the first part 834 may correspond to a proximal region of the housing 802. The first part 834 may be referred to as a mount. In other examples, the first part 834 may be referred to as a first part of a shell. The adhesive patch 106 is attached to the base surface 804 in a similar manner to OBD 100-1. The adhesive patch 106 may be attached to the first part 834 before or after assembly of the first part 834 and the second part 836. The first part 834 also comprises at least a portion of the side surface 810. In this embodiment, the first part 834 comprises a portion of the side surface 810 (e.g., approximately half). In this embodiment, the first portion of side surface 810 on first part 834 terminates in a rim (e.g., a circular rim).

In this embodiment, the base surface 804 is circular, as in OBD 100-1. Similarly, the side surface 810 is perpendicular to the base surface 804 and extends circumferentially, as in OBD 100-1. The first part 834 is hollow defining an interior space defined by the base surface 804 and a portion of the side surface 810. The first part 834 has an internal surface on an opposite side of the first part 834 to the base surface 804 and the side surface 810. The first part 834 also comprises sensor aperture 112. The first part 834 also comprises a portion of a sensor passageway 126, terminating in the sensor aperture 112 in the base surface 804. In this embodiment, the sensor passageway 126 is cylindrical, and the first part 834 comprises a cylindrical passageway wall extending from the base surface 804 at the central axis A, the cylindrical passageway wall defining the sensor passageway 126. In other examples, the sensor aperture 112 and/or the sensor passageway 126 may have a different shape or location, such as described in relation to OBD 100-1.

The sensor electronics 122 can be assembled onto the first part 834. The analyte sensor 161 can also be assembled onto the first part 834. For example, the first portion 118 of the analyte sensor 161 may be placed into contact with the sensor electronics 122, and then the combined analyte sensor 161 and sensor electronics 122 may be assembled onto the first part 834. In other examples, the analyte sensor 161 may be assembled to the first part 834 after the sensor electronics 122. In other examples, the analyte sensor 161 may be assembled to the first part 834 before the sensor electronics 122.

The sensor electronics 122 may be assembled onto the first part 834 with the cylindrical passageway wall of the sensor passageway 126 passing through an aperture in the sensor electronics 122. The second portion 120 of the analyte sensor 161 may pass through the sensor passageway 126 and through the sensor aperture 112.

In this embodiment, the second part 836 comprises the top surface 808. The second part 836 may be referred to as a top part, an upper part, a distal part, a non-adhesive-contacting part, or a non-skin-facing part. For example, the second part 836 may correspond to a top (or top portion) of the housing 802. For example, the second part 836 may correspond to a distal region of the housing 802. In other examples, the second part 836 may be referred to as a second part of a shell. The second part 836 also comprises at least a portion of the side surface 810. In this embodiment, the second part 836 comprises a portion of the side surface 810 (e.g., approximately half). The second portion of the side surface 810 on the second part 836 terminates in a rim (e.g., a circular rim). The housing 802 may comprise a first side surface 810 on the first part 834 and a second side surface 810 on the second part 836, with the first side surface 810 aligned with the second side surface 810 (e.g., together providing a side or periphery of the housing 802).

In this embodiment, the top surface 808 is circular, as in OBD 100-1. Similarly, the side surface 810 is perpendicular to the top surface 808 and extends circumferentially, as in OBD 100-1. The second part 836 is hollow defining an interior space defined by the top surface 808 and a portion of the side surface 810. The second part 836 has an internal surface on an opposite side of the second part 836 to the top surface 808 and the side surface 810. The second part 836 also comprises a sharp aperture 124 which is similar to the sharp aperture 124. The second part 836 also comprises a portion of a sensor passageway 126, terminating in the sharp aperture 124 in the top surface 808. In this embodiment, the second part 836 comprises a cylindrical passageway wall extending from the top surface 808 at the central axis A, the cylindrical passageway wall defining the sensor passageway 126. In other examples, the sharp aperture 124 and/or the sensor passageway 126 may have a different shape or location, such as described in relation to OBD 100-1.

The second part 836 can be assembled onto the first part 834. For example, the second part 836 can be assembled after the analyte sensor 161 and the sensor electronics 122 have been assembled onto the first part 834. The second part 836 is attached to the first part 834 to seal the housing 802. In particular, the circular rim of the side surface 810 on the second part 836 is configured to engage and be sealed to the circular rim of the side surface 810 on the first part 834. In this manner, the first and second portions of the side surface 810 are connected. The sensor electronics 122 and the analyte sensor 161 are then enclosed by the first part 834 and the second part 836, in a similar manner to OBD 100-1. The cylindrical passageway wall sections of the first part 834 and the second part 836 may also be attached to complete the sensor passageway 126. For example, the cylindrical passageway wall of the second part 836 may extend through the aperture in the sensor electronics 122 to meet the cylindrical passageway wall of the first part 834 for attachment. The sensor electronics 122 can then be arranged around the sensor passageway 126. In some examples, the sensor passageway 126 is provided at least in part by a sensor connector (e.g., sensor connector 940 or sensor connector 1040).

The first part 834 and the second part 836 may be attached together by an adhesive (e.g., an ultraviolet-cured adhesive), a snap fit or interference fit, threading, sonic welding, mechanical fasteners (e.g., screws), or any other attachment means. Upon assembly, a sealed interface may be formed. In some examples, a gasket or other seal may be positioned at or near the interface (e.g., between the circular rims), with the attachment compressing the gasket to form the sealed interface. In some examples, the sealed housing 802 may be water-resistant or waterproof. For example, the housing 802 may withstand immersion in about one (1) meter of water for up to about 30 minutes.

In general, OBD 100-8 is similar to OBD 100-1, except that the housing 802 is formed from a first part 834 and a second part 836 which are connected together. For example, the first part 834 may be made of a different material to the second part 836. In this manner, the base surface 804 may be made of a different material to the top surface 804.

In this embodiment, the first part 834 is formed by two-shot molding. In particular, the first part 834 is molded from plastic and, for example, a seal is over-molded onto the plastic. The first part 834 may be injection molded and formed of plastic, such as polycarbonate or ABS, and the seal may be an elastomer. The sensor electronics 122 and the analyte sensor 161 are then assembled by arranging the components on the first part 834 and securing by adhesive (e.g., UV-cure adhesive). The second part 836 is then secured to the first part 834, enclosing the sensor electronics 122 and the analyte sensor 161. The second part 836 may be made from plastic material, such as polycarbonate or ABS. The second part 836 is secured to the first part 834 using an adhesive (e.g., UV-cure adhesive). In other examples, other manufacturing techniques and other materials may be used. For example, the seal may be omitted.

In some examples, different surfaces may be integrally formed. For example, instead of providing a separate first part 834 and second part 836 which are attached together, the base surface 804 and the top surface 808 may be integrally formed together. For example, the base surface 804, the side surface 810, and the top surface 808 may be integrally formed. Thus, the housing 802 may be molded as one piece. For example, the housing 802 may be molded over the components inside.

In another example, the base surface 804 may be integrally formed with the side surface 810 (forming a first part 834) and then the top surface 808 is formed separately (forming a second part 836) and then attached to the combined base surface 804 and side surface 810. In another example, the side surface 110 may be integrally formed with the top surface 108 (forming a second part 836) and then the base surface 104 is formed separately (forming a first part 834) and then attached to the combined side surface 110 and top surface 108.

OBD 100-8 comprises an analyte sensor 161 and sensor electronics 122 in a similar manner to OBD 100-1. In other examples, variations to the analyte sensor 161 and/or the sensor electronics 122 described herein may be applied to OBD 100-8.

OBD 100-8 is shown and described with a partially implantable sensor but can be configured to operate with a wholly implantable sensor or wholly ex vivo sensor, in which case the adhesive aperture, sensor aperture, sensor passageway, and sharp aperture can be omitted.

In other examples, the base surface 804 and the top surface 808 may have different shapes, such as the square shape shown in OBD 100-2, or the other shapes described with reference to OBD 100. The first part 834 and the second part 836 may be applied to different shapes, such as the square shape or other shape. For example, the rims joining the first part 834 and the second part 836 may be square, or other shape.

In other examples, the side surface 810 may not be perpendicular to the base surface 804. For example, the side surface 810 may be angled, such as in OBD 100-3, or curved, such as in OBD 100-4. The first part 834 and the second part 836 may each comprise a portion of the angled or curved side surface 810.

In other examples, the housing 802 may not comprise a separate side surface 810, and instead the top surface 808 may connect to the base surface 804, such as in OBD 100-5. The first part 834 and the second part 836 may each comprise a portion of the top surface 808. Alternatively, the first part 834 may comprise the base surface 804 and the second part 836 may comprise the top surface 808.

In other examples, the housing 802 may comprise an intermediate surface. For example, the housing 802 may comprise an angled intermediate surface, such as in OBD 100-6, or a curved intermediate surface, such as in OBD 100-7. For example, the second part 836 may comprise the intermediate surface (e.g., if connected to the top surface 808). An example implementation is shown in FIG. 1N-1. In other examples, the first part 834 may comprise the intermediate surface (e.g., if connected to the base surface 804).

### On-Body Device 100-9

Referring to FIGS. 1J-1 and 1J-2, an OBD 100-9 is shown according to an embodiment. OBD 100-9 may be the same as OBD 100-1 of FIGS. 1B-1 to 1B-3, and may include the same features, except as explained below. The optional features and alternatives described in relation to OBD 100-1 may similarly be applied to OBD 100-9 in a corresponding manner. In some examples, OBD 100-9 may be similar to OBD 100-2 of FIGS. 1C-1 and 1C-2, OBD 100-3 of FIG. 1D, OBD 100-4 of FIG. 1E, OBD 100-5 of FIG. 1F, OBD 100-6 of FIG. 1G, OBD 100-7 of FIG. 1H, or OBD 100-8 of FIG. 1I, and features described in relation to those embodiments may be applied to OBD 100-9.

In this embodiment, OBD 100-9 is the same as OBD 100-1, except that OBD 100-9 has a housing 902 with a different configuration compared to the housing 102. In particular, the housing 902 comprises a recess 938. The recess 938 is arranged in the top of the housing 902. Specifically, the top surface 908 comprises an opening defining an opening of the recess 938. In this embodiment, the central axis A passes through the recess 938. In other examples, the recess 938 may be positioned at a different location. OBD 100-9 also comprises a base surface 904 and a side surface 910 as in OBD 100-1. The adhesive patch 106 is attached to the base surface 904 in a similar manner to OBD 100-1.

The recess 938 extends into the housing 902 (e.g., into the height of the housing 902 in a direction parallel to the central axis A) exposing the sensor electronics 122. For example, sensor contacts of the sensor electronics 122 may be arranged at the bottom of the recess 938.

The housing 902 comprises a first section of the sensor passageway 126. The first section of the sensor passageway 126 is connected to the recess 938. The first section of the sensor passageway 126 terminates in the sensor aperture 112 in the base surface 904. Thus, the first section of the sensor passageway 126 extends between the recess 938 and the sensor aperture 112.

OBD 100-9 also comprises a sensor connector 940. The sensor connector 940 is configured to be inserted into the recess 938. Thus, the sensor connector 940 may initially be separate from the housing 902. The sensor connector 940 has a complementary size and shape to be received into the recess 938. The sensor connector 940 comprises the analyte sensor 161. The sensor connector 940 may also comprise conductive contacts for coupling the analyte sensor 161 to the sensor electronics 122. In some examples, the sensor connector 940 is configured to securely retain the analyte sensor 161 in the recess 938 when positioned in the recess 938. The conductive contacts may comprise a conductive elastomeric material to facilitate electrical connection when the elastomeric contacts are compressed. In some examples, the sensor connector 940 may include some electronics components of the sensor electronics 122.

In this embodiment, the sensor connector 940 comprises a second section of the sensor passageway 126. The analyte sensor 161 extends through a passageway wall of the sensor passageway 126 and into the sensor passageway 126. The analyte sensor 161 then protrudes out from the sensor passageway 126 in the proximal direction. When the sensor connector 940 is assembled in the recess 938, the second section of the sensor passageway 126 is connected to the first section of the sensor passageway 126.

In this embodiment, the sensor connector 940 is assembled into the recess 938 during the insertion process. Thus, during manufacture, the sensor connector 940 is separate from the housing 902. This may allow the analyte sensor 161 to be sterilized separately from the sensor electronics 122. For example, the sensor connector 940 may be arranged within the inserter (e.g., in a distal position). For example, the sensor connector 940 may be positioned (e.g., with a sharp) inside the inserter. During the insertion process performed by the user, the housing 902 is placed on the skin, for example being held at the bottom of the inserter housing of an inserter (e.g., inserter 60). The housing 902 may be provided separately from the inserter and may be coupled to the bottom of the inserter by the user, or the housing 902 may already be provided at the bottom of the inserter. The sensor connector 940 can then be advanced (e.g., by the inserter) relative to the housing 902 (e.g., from a distal position to a proximal position) until the sensor connector 940 is positioned into the recess 938. In particular, the sensor connector 940 is advanced in a direction parallel to the central axis A. The housing 902 may be configured to engage with the sensor connector 940. For example, the sensor connector 940 may be retained within the recess 938, such as by a snap fit connection.

As shown in FIG. 1J-2, when the sensor connector 940 is positioned in the recess 938, the analyte sensor 161 extends through the first section of the sensor passageway 126 and out of the sensor aperture 112 for insertion into the body 5. For example, when the housing 902 is placed on the skin and the sensor connector 940 is advanced into the recess 938, the second portion 120 of the analyte sensor 161 is inserted into the body 5. For example, a sharp of the inserter may be used to insert the analyte sensor 161. Thus, the sharp may be received through the sensor connector 940 (e.g., through the sharp aperture 124 and through the second section of the sensor passageway 126). The sharp may be inserted through the sensor connector 940 by passing through the plane of the top surface 908 (e.g., through the opening of the recess 938) and through the second section of the sensor passageway 126 of the sensor connector 940. The sharp may then be withdrawn from the sensor connector 940 after insertion of the analyte sensor 161. In this manner, the sensor connector 940 may be assembled into the recess 938 with the same action as inserting the analyte sensor 161 into the body 5 (e.g., a single step process).

When the sensor connector 940 is positioned in the recess 938, the analyte sensor 161 is in contact with the sensor electronics 122. In particular, the first portion 118 of the analyte sensor 161 is in electrical contact with the sensor electronics 122. The sensor connector 940 may be coupled to the housing 902 at the recess 938 to form a seal to prevent ingress of contaminants (e.g., moisture) into the sensor electronics 122.

In other examples, the sensor connector 940 may be inserted into the recess 938 during manufacture. In other examples, the sensor connector 940 may be inserted into the recess 938 after manufacturer but prior to the insertion process, for example by the user.

OBD 100-9 comprises an analyte sensor 161 and sensor electronics 122 in a similar manner to OBD 100-1. In other examples, variations to the analyte sensor 161 and/or the sensor electronics 122 described herein may be applied to OBD 100-9.

OBD 100-9 is shown and described with a partially implantable sensor but can be configured to operate with a wholly implantable sensor or wholly ex vivo sensor, in which case the adhesive aperture, sensor aperture, sensor passageway, and sharp aperture can be omitted.

In other examples, the base surface 904 and the top surface 908 may have different shapes, such as the square shape shown in OBD 100-2, or the other shapes described with reference to OBD 100. The sensor connector 940 and the recess 938 may be applied to different shapes, such as the square shape or other shape.

In other examples, the side surface 910 may not be perpendicular to the base surface 904. For example, the side surface 910 may be angled, such as in OBD 100-3, or curved, such as in OBD 100-4.

In other examples, the housing 902 may not comprise a separate side surface 910, and instead the top surface 908 may connect to the base surface 904, such as in OBD 100-5.

In other examples, the housing 902 may comprise an intermediate surface. For example, the housing 902 may comprise an angled intermediate surface, such as in OBD 100-6, or a curved intermediate surface, such as in OBD 100-7.

In other examples, the housing 902 may comprise a first part and a second part attached together during manufacture, such as in OBD 100-8. For example, the second part may comprise the recess 938 for receiving the sensor connector 940.

### On-Body Device 100-10

Referring to FIGS. 1K-1 and 1K-2, an OBD 100-10 is shown according to an embodiment. OBD 100-10 may be the same as OBD 100-1 of FIGS. 1B-1 to 1B-3, and may include the same features, except as explained below. The optional features and alternatives described in relation to OBD 100-1 may similarly be applied to OBD 100-10 in a corresponding manner. In some examples, OBD 100-10 may be similar to OBD 100-2 of FIGS. 1C-1 and 1C-2, OBD 100-3 of FIG. 1D, OBD 100-4 of FIG. 1E, OBD 100-5 of FIG. 1F, OBD 100-6 of FIG. 1G, OBD 100-7 of FIG. 1H, or OBD 100-8 of FIG. 1I, and features described in relation to those embodiments may be applied to OBD 100-10.

In this embodiment, OBD 100-10 is the same as OBD 100-1, except that OBD 100-10 has a housing 1002 with a different configuration compared to the housing 102. In particular, the housing 1002 comprises a recess 1038, in a similar manner to OBD 100-9. Features of OBD 100-9 (especially in relation to the recess 938 and the sensor connector 940) may be applied correspondingly to OBD 100-10. In contrast to the recess 938 of OBD 100-9, the recess 1038 is arranged in the bottom of the housing 1002. Specifically, the base surface 1004 comprises an opening defining an opening of the recess 1038. In this embodiment, the central axis A passes through the recess 1038. In other examples, the recess 1038 may be positioned at a different location. OBD 100-10 also comprises a top surface 1008 and a side surface 1010 as in OBD 100-1. The adhesive patch 106 is attached to the base surface 1004 in a similar manner to OBD 100-1, except as described below.

The recess 1038 extends into the housing 1002 (e.g., into the height of the housing 1002 in a direction parallel to the central axis A) exposing the sensor electronics 122. For example, sensor contacts of the sensor electronics 122 may be arranged at an edge of the recess 1038.

The housing 1002 comprises a first section of the sensor passageway 126(1). The first section of the sensor passageway 126(1) is connected to the recess 1038. The first section of the sensor passageway 126(1) terminates in the sharp aperture 124. Thus, the first section of the sensor passageway 126(1) extends between the recess 1038 and the sharp aperture 124.

OBD 100-10 also comprises a sensor connector 1040. The sensor connector 1040 is configured to be inserted into the recess 1038. Thus, the sensor connector 1040 may initially be separate from the housing 1002. The sensor connector 1040 has a complementary size and shape to be received into the recess 1038. The sensor connector 1040 comprises the analyte sensor 161. The sensor connector 1040 may also comprise conductive contacts for coupling the analyte sensor 161 to the sensor electronics 122. In some examples, the sensor connector 1040 is configured to securely retain the analyte sensor 161 in the recess 1038 when positioned in the recess 1038. In some examples, the sensor connector 1040 may include some electronics components of the sensor electronics 122.

In this embodiment, the sensor connector 1040 comprises a second section of the sensor passageway 126(2). The analyte sensor 161 extends through a passageway wall of the second section of the sensor passageway 126(2) and along the section 126(2). The analyte sensor 161 then protrudes out from the second section of the sensor passageway 126(2). When the sensor connector 1040 is assembled in recess 1038, the second section of sensor passageway 126(2) is connected to the first section of sensor passageway 126(1).

In this embodiment, the sensor connector 1040 is assembled into the recess 1038 by the user before the insertion process. Thus, during manufacture, the sensor connector 1040 is separate from the housing 1002. This may allow the analyte sensor 161 to be sterilized separately from the sensor electronics 122. For example, the housing 1002 may be arranged (e.g., with a sharp) within the inserter (e.g., inserter 60). Before the insertion process performed by the user, the sensor connector 1040 may be loaded into the inserter (e.g., from a separate package) to advance the sensor connector 1040 relative to the housing 1002 (e.g., from a proximal position to a distal position) until the sensor connector 1040 is positioned into the recess 1038. In particular, the sensor connector 1040 is advanced in a direction parallel to the central axis A. The housing 1002 may be configured to engage with the sensor connector 1040. For example, the sensor connector 1040 may be retained within the recess 1038, such as by a snap fit connection.

The assembly of the sensor connector 1040 into the housing 1002 may be performed as a step before insertion of the analyte sensor 161. After the sensor connector 1040 is positioned in the recess 1038, as shown in FIG. 1K-2, the analyte sensor 161 may be inserted into the body 5. For example, OBD 100-10 (the combined housing 1002 and sensor connector 1040) may be arranged at a distal position within the inserter. OBD 100-10 may then be advanced within the inserter in a proximal direction towards the skin.

When the sensor connector 1040 is positioned in the recess 1038, the analyte sensor 161 extends in a proximal direction through the second section of the sensor passageway 126(2) and out of the sensor aperture 112 for insertion into the body 5. The analyte sensor 161 may extend through the second section of the passageway 126(2) of the sensor connector 1040 and through the plane of the base surface 1004 (e.g., through the opening of the recess 1038). For example, when OBD 100-10 is advanced until the base surface 1004 contacts the skin, the second portion 120 of the analyte sensor 161 is inserted into the body 5. For example, a sharp of the inserter may be used to insert the analyte sensor 161. Thus, the sharp may be received through the sensor connector 1040 (e.g., through the sharp aperture 124 and through the first section of the sensor passageway 126(1)). The sharp may then be withdrawn from the sensor connector 1040 after insertion of the analyte sensor 161.

When the sensor connector 1040 is positioned in the recess 1038, the analyte sensor 161 is in contact with the sensor electronics 122. In particular, the first portion 118 of the analyte sensor 161 is in electrical contact with the sensor electronics 122. The sensor connector 1040 may be coupled to the recess 1038 to form a seal to prevent ingress of contaminants (e.g., moisture) into the sensor electronics 122. In some examples, the analyte sensor 161 is positioned on a lower surface of the sensor electronics 122 (e.g., an underside of a PCB, such as shown in FIG. 1N-2). For example, the sensor connector 1040 may be positioned in the recess 1038 between the sensor electronics 122 and the base surface 1004.

In this embodiment, the adhesive patch 106 comprises an adhesive aperture 114 corresponding to the recess 1038. This allows the sensor connector 1040 to be inserted into the recess 1038. The adhesive aperture 114 is therefore larger than the adhesive aperture 114 of the adhesive patch 106 of FIG. 1B-1. In some examples, the sensor connector 1040 comprises an adhesive on the proximal surface providing adhesive contact with the skin after being inserted into the recess 1038. This adhesive may have an aperture for receiving the analyte sensor 161 therethrough. In other examples, the sensor connector 1040 does not comprise an adhesive, and the adhesive aperture 1014 extends around the recess 1038/sensor connector 1040.

In other examples, the sensor connector 1040 may be inserted into the recess 1038 during manufacture.

OBD 100-10 comprises an analyte sensor 161 and sensor electronics 122 in a similar manner to OBD 100-1. In other examples, variations to the analyte sensor 161 and/or the sensor electronics 122 described herein may be applied to OBD 100-10.

OBD 100-10 is shown and described with a partially implantable sensor but can be configured to operate with a wholly implantable sensor or wholly ex vivo sensor, in which case the adhesive aperture, sensor aperture, sensor passageway, and sharp aperture can be omitted.

In other examples, the base surface 1004 and the top surface 1008 may have different shapes, such as the square shape shown in OBD 100-2, or the other shapes described with reference to OBD 100. The sensor connector 1040 and recess 1038 may be applied to different shapes, such as the square shape or other shape.

In other examples, the side surface 1010 may not be perpendicular to the base surface 1004. For example, the side surface 1010 may be angled, such as in OBD 100-3, or curved, such as in OBD 100-4.

In other examples, the housing 1002 may not comprise a separate side surface 1010, and instead the top surface 1008 may connect to the base surface 1004, such as in OBD 100-5.

In other examples, the housing 1002 may comprise an intermediate surface. For example, the housing 1002 may comprise an angled intermediate surface, such as in OBD 100-6, or a curved intermediate surface, such as in OBD 100-7.

In other examples, the housing 1002 may comprise a first part and a second part attached together during manufacture, such as in OBD 100-8. For example, the first part may comprise the recess 1038 for receiving the sensor connector 1040.

### On-Body Device 100-11

Referring to FIGS. 1L-1 and 1L-2, an OBD 100-11 is shown according to an embodiment. OBD 100-11 may be the same as OBD 100-1 of FIGS. 1B-1 to 1B-3, and may include the same features, except as explained below. The optional features and alternatives described in relation to OBD 100-1 may similarly be applied to OBD 100-11 in a corresponding manner. In some examples, OBD 100-11 may be similar to OBD 100-2 of FIGS. 1C-1 and 1C-2, OBD 100-3 of FIG. 1D, OBD 100-4 of FIG. 1E, OBD 100-5 of FIG. 1F, OBD 100-6 of FIG. 1G, OBD 100-7 of FIG. 1H, or OBD 100-8 of FIG. 1I, and features described in relation to those embodiments may be applied to OBD 100-11.

In this embodiment, OBD 100-11 is the same as OBD 100-1, except that OBD 100-11 has a housing 1102 with a different configuration compared to the housing 102. In particular, the housing 1102 comprises a recess 1138, in a similar manner to OBD 100-9 and OBD 100-10. Features of OBD 100-9 and OBD 100-10 (especially in relation to the recess 938 and the recess 1038) may be applied correspondingly to OBD 100-11. The recess 1138 is arranged in the bottom of the housing 1102, in a similar manner to OBD 100-10. Specifically, the base surface 1104 comprises an opening defining an opening of the recess 1138. In this embodiment, the central axis A passes through the recess 1138. In other examples, the recess 1138 may be positioned at a different location. OBD 100-11 also comprises a top surface 1108 and a side surface 1110 as in OBD 100-1. The adhesive patch 106 is attached to the base surface 1104 in a similar manner to OBD 100-1, except as described below.

In this embodiment, the recess 1138 is under (e.g. proximal of) the sensor electronics 122 (e.g., between the sensor electronics 122 and the base surface 1104. In this embodiment, the analyte sensor 161 may be positioned in the recess 1138 during manufacture. In particular, there is not a separate sensor connector (such as the sensor connector 940 or the sensor connector 1040). Instead, the analyte sensor 161 may be directly connected to the sensor electronics 122 in the recess 1138. For example, the analyte sensor 161 may be attached to the sensor electronics 122 via a conductive connection, such as by soldering or glue.

In this embodiment, OBD 100-11 also comprises a filling material 1142. The filling material 1142 is configured to assist retaining the analyte sensor 161 in the recess 1138 and to prevent ingress of contaminants (e.g., moisture) into the sensor electronics 122. For example, the filling material 1142 may be molded or potted into the recess 1138. For example, the filling material 1142 may be impermeable to water (e.g., liquid water, and optionally also impermeable to water vapor). The filling material 1142 may not be electrically conductive to avoid shorting the sensor electronics 122. In some examples, the filling material 1142 at least partially fills the recess 1138. For example, the filling material 1142 may be placed within the recess 1138 to seal the analyte sensor 161 but allowing space for the sensor passageway 126 for insertion and retraction of the sharp. The analyte sensor 161 may extend from the filling material 1142 into the sensor passageway 126.

In this embodiment, the adhesive patch 106 comprises an adhesive aperture 114 corresponding to the recess 1138. This allows the analyte sensor 161 and the filling material 1142 to be inserted into the recess 1138. The adhesive aperture 114 is therefore larger than the adhesive aperture 114 of FIG. 1B-1. In some examples, an adhesive is applied on the proximal surface of the filling material 1142, providing adhesive contact with the skin after being inserted into the recess 1138. In other examples, the filling material 1142 does not comprise an adhesive, and adhesive aperture 114 extends around the recess 1138.

OBD 100-11 comprises an analyte sensor 161 and sensor electronics 122 in a similar manner to OBD 100-1. In other examples, variations to the analyte sensor 161 and/or the sensor electronics 122 described herein may be applied to OBD 100-11.

OBD 100-11 is shown and described with a partially implantable sensor but can be configured to operate with a wholly implantable sensor or wholly ex vivo sensor, in which case the adhesive aperture, sensor passageway, and sharp aperture can be omitted.

OBD 1110 allows the housing 1102 to be formed integrally. For example, the housing 1102 may be formed by over-molding, such as over-molding over the sensor electronics 122. For example, two parts (such as in OBD 100-8) may not be required. The integrated (e.g., over-molded) assembly may be applied to the various different housing shapes and configurations described herein. Similarly, OBD 100-11 having the recess 1138 and filling material 1142 may be implemented with different manufacturing techniques, such as the housing with two parts (e.g., see OBD 100-8).

In other examples, the base surface 1104 and the top surface 1108 may have different shapes, such as the square shape shown in OBD 100-2, or the other shapes described with reference to OBD 100. The recess 1138 and filling material 1142 may be applied to different shapes, such as the square shape or other shape.

In other examples, the side surface 1110 may not be perpendicular to the base surface 1104. For example, the side surface 1110 may be angled, such as in OBD 100-3, or curved, such as in OBD 100-4.

In other examples, the housing 1102 may not comprise a separate side surface 1110, and instead the top surface 1108 may connect to the base surface 1104, such as in OBD 100-5.

In other examples, the housing 1102 may comprise an intermediate surface. For example, the housing 1102 may comprise an angled intermediate surface, such as in OBD 100-6, or a curved intermediate surface, such as in OBD 100-7.

In other examples, the housing 1102 may comprise a first part and a second part attached together during manufacture, such as in OBD 100-8. For example, the first part may comprise the recess 1138 for receiving the analyte sensor 161 and the filling material 1142.

### On-Body Device 100-12

Referring to FIGS. 1M-1 and 1M-2, an OBD 100-12 is shown according to an embodiment. OBD 100-12 may be the same as OBD 100-1 of FIGS. 1B-1 to 1B-3, and may include the same features, except as explained below. The optional features and alternatives described in relation to OBD 100-1 may similarly be applied to OBD 100-12 in a corresponding manner. In some examples, OBD 100-12 may be similar to OBD 100-2 of FIGS. 1C-1 and 1C-2, OBD 100-3 of FIG. 1D, OBD 100-4 of FIG. 1E, OBD 100-5 of FIG. 1F, OBD 100-6 of FIG. 1G, OBD 100-7 of FIG. 1H, or OBD 100-8 of FIG. 1I, and features described in relation to those embodiments may be applied to OBD 100-12.

In this embodiment, OBD 100-12 is the same as OBD 100-1, except that OBD 100-12 has a housing 1202 with a different configuration compared to the housing 102. In particular, the housing 1202 comprises a recess 1238. Unlike OBD 100-9 and OBD 100-10, the analyte sensor 161 is positioned in the housing 1202 (e.g., at the bottom of the recess 1238). The recess 1238 is arranged in the top of the housing 1202. In other examples, the recess 1238 may be positioned at a different location. OBD 100-12 also comprises a base surface 1204, a top surface 1208, and a side surface 1210 as in OBD 100-1. The adhesive patch 106 is attached to the base surface 1204 in a similar manner to OBD 100-1.

The recess 1238 is configured to receive sensor electronics 122. When the sensor electronics 122 are positioned into the recess 1238, sensor electronics 122 are in electrical contact with the analyte sensor 61.

In this embodiment, the sensor electronics 122 are inserted into the recess 1238 after the analyte sensor 161 has been inserted into the body 5. As shown in FIG. 1M-1, the analyte sensor 161 is inserted into the body 5. For example, the analyte sensor 161 may be inserted using an inserter (e.g., inserter 60). In some examples, the analyte sensor 161 may be inserted with the housing 1202. For example, the analyte sensor 161 may be coupled to the housing 1202 within the inserter, and then the housing 1202 may be advanced into contact with the skin, at which point the analyte sensor 161 is inserted into the body 5. In other examples, the housing 1202 may be positioned on the skin, and the analyte sensor 161 may be inserted through the housing 1202 (e.g., by an inserter).

After the analyte sensor 161 has been inserted into the body 5, the sensor electronics 122 can be inserted into the recess 1238. In this manner, the sensor electronics 122 does not have an aperture for the sharp, since the analyte sensor 161 is inserted before the sensor electronics 122 are coupled to the housing 1202. OBD 100-12 may be referred to as a two-part OBD or a two-part system. The sensor electronics 122 may be replaceable to extend the wear duration of OBD. The housing 1202 may be referred to as a receptable and may be configured to receive the sensor electronics 122. The sensor electronics 122 may be arranged in a separate sealed housing which is retained by the housing 1202, or the sensor electronics 122 may be sealed by engagement with the housing 1202. For example, the housing 1202 may comprise a base (e.g., a base portion) such as a first part, and a top (e.g., a top portion) such as a second part comprising the sensor electronics 122 may be coupled to the base after insertion of the analyte sensor 161.

OBD 100-12 comprises an analyte sensor 161 and sensor electronics 122 in a similar manner to OBD 100-1. In other examples, variations to the analyte sensor 161 and/or the sensor electronics 122 described herein may be applied to OBD 100-12.

OBD 100-12 is shown and described with a partially implantable sensor but can be configured to operate with a wholly implantable sensor or wholly ex vivo sensor, in which case the adhesive aperture, sensor aperture, sensor passageway, and sharp aperture can be omitted.

In other examples, the base surface 1204 and the top surface 1208 may have different shapes, such as the square shape shown in OBD 100-2, or the other shapes described with reference to OBD 100. The two-part system of OBD 100-12 may be applied to different shapes, such as the square shape or other shape.

In other examples, the side surface 1210 may not be perpendicular to the base surface 1204. For example, the side surface 1210 may be angled, such as in OBD 100-3, or curved, such as in OBD 100-4.

In other examples, the housing 1202 may not comprise a separate side surface 1210, and instead the top surface 1208 may connect to the base surface 1204, such as in OBD 100-5.

In other examples, the housing 1202 may comprise an intermediate surface. For example, the housing 1202 may comprise an angled intermediate surface, such as in OBD 100-6, or a curved intermediate surface, such as in OBD 100-7.

In other examples, the housing 1202 may comprise a first part and a second part attached together during manufacture, such as in OBD 100-8. For example, the second part may comprise the recess 1238 for receiving the sensor electronics 122.

### On-Body Device 100-13

Referring to FIG. 1N-1, an OBD 100-13 is shown according to an embodiment. OBD 100-13 may be the same as OBD 100-1 of FIGS. 1B-1 to 1B-3, and may include the same features, except as explained below. The optional features and alternatives described in relation to OBD 100-1 may similarly be applied to OBD 100-13 in a corresponding manner. In some examples, OBD 100-13 may be similar to OBD 100-2 of FIGS. 1C-1 and 1C-2, OBD 100-3 of FIG. 1D, OBD 100-4 of FIG. 1E, OBD 100-5 of FIG. 1F, OBD 100-6 of FIG. 1G, OBD 100-7 of FIG. 1H, OBD 100-8 of FIG. 1I, OBD 100-9 of FIGS. 1J-1 and 1J-2, OBD 100-10 of FIGS. 1K-1 and 1K-2, OBD 100-11 of FIGS. 1L-1 and 1L-2, or OBD 100-12 of FIGS. 1M-1 and 1M-2, and features described in relation to those embodiments may be applied to OBD 100-13.

OBD 100-13 comprises a housing 1302. The housing 1302 comprises a first part 1334 and a second part 1336, in a similar manner to OBD 100-8. The first part 1334 includes a base surface (not shown). OBD 100-13 comprises sensor electronics 122 including a PCB. Various electronic components are shown such as a battery and an antenna. The electronic components are on a distal side of the PCB. The sensor electronics 122 are assembled onto the first part 1334. The second part 1336 can then be assembled onto the first part 1334, such as described in relation to OBD 100-8 (e.g., see FIG. 1I).

The analyte sensor 161A of FIG. 1Q-1 (described further herein) is shown assembled to the sensor electronics 122 in OBD 100-13. In particular, the sensor contacts on the side surface of the first portion 118 are in electrical contact with a connector on the sensor electronics 122. In this manner, the first portion 118 has a side surface in a plane perpendicular to the PCB.

The second part 1336 comprises a top surface 1308. The top surface 1308 comprises a sharp aperture 124. In this embodiment, the sharp aperture 124 is not aligned with a central axis A, and instead is offset as described herein. In other examples, the sharp aperture 124 may be central. In this embodiment, the housing 1302 comprises a side wall 1310. In particular, a first section of the side wall 1310 is on the first part 1334 and a second section is on the second part 1336. The second part 1336 also comprises a curved intermediate surface in a similar manner to OBD 100-7 (e.g., see FIG. 1H). In other examples, the intermediate surface may be angled (e.g., see FIG. 1G), or may be omitted.

### On-Body Device 100-14

Referring to FIG. 1N-2, an OBD 100-14 is shown according to an embodiment. OBD 100-14 may be the same as OBD 100-1 of FIGS. 1B-1 to 1B-3, and may include the same features, except as explained below. The optional features and alternatives described in relation to OBD 100-1 may similarly be applied to OBD 100-14 in a corresponding manner. In some examples, OBD 100-14 may be similar to OBD 100-2 of FIGS. 1C-1 and 1C-2, OBD 100-3 of FIG. 1D, OBD 100-4 of FIG. 1E, OBD 100-5 of FIG. 1F, OBD 100-6 of FIG. 1G, OBD 100-7 of FIG. 1H, OBD 100-8 of FIG. 1I, OBD 100-9 of FIGS. 1J-1 and 1J-2, OBD 100-10 of FIGS. 1K-1 and 1K-2, OBD 100-11 of FIGS. 1L-1 and 1L-2, OBD 100-12 of FIGS. 1M-1 and 1M-2, or OBD 100-13 of FIG. 1N-1, and features described in relation to those embodiments may be applied to OBD 100-14.

OBD 100-14 comprises a housing 1402. The housing 1402 comprises a first part (not shown) and a second part 1436, in a similar manner to OBD 100-8 and OBD 100-13. OBD 100-14 comprises sensor electronics 122 including a PCB. Various electronic components are shown such as a battery and an antenna. The electronic components are on a proximal side of the PCB. The sensor electronics 122 are assembled onto the second part 1436. The first part can then be assembled onto the second part 1434, such as described in relation to OBD 100-8 (e.g., see FIG. 1I) or OBD 100-13 (e.g., see FIG. 1N-1).

The analyte sensor 161A of FIG. 1Q-2 (described further herein) is coupled to the sensor electronics 122. In particular, the analyte sensor 161A is coupled to a proximal side (e.g., an underside) of the PCB. In this manner, the first portion 118 has a side surface in a plane parallel to the PCB. This allows the sensor contacts of the analyte sensor 161A to be coupled to the PCB. In this embodiment, the analyte sensor 161A is coupled to the sensor electronics 122 using a sensor connector 1440. The sensor connector 1440 is inserted into the housing 1402 from below, in a similar manner to OBD 100-10 (e.g., see FIGS. 1K-1 and 1K-2). Components may be mounted on one or both sides of the PCB as desired. Here, the upper surface of the PCB has a power source 320 configured as a coin cell battery mounted thereon. The lower surface of the PCB has a semiconductor chip 1437 mounted thereon where the semiconductor chip may include both wireless communication electronics 312 (e.g., transceiver 316) and processing electronics 315 integrated in chip 1437. The antenna 314 can be included in a different location external to chip 1437.

The second part 1436 comprises a top surface (not shown). The top surface comprises a sharp aperture (not shown). In this embodiment, the sharp aperture is aligned with a central axis A. In other examples, the sharp aperture may be off-center as described herein. In this embodiment, the housing 1402 comprises a side wall 1410. In particular, a first section (not shown) of the side wall 1410 is on the first part and a second section of the side wall 1410 is on the second part 1436.

As shown in FIG. 1N-2, the housing 1402 comprises three supporting posts that extend from an interior surface of the second part 1436 (e.g., opposite to the top surface). The supporting posts pass through the sensor electronics 122 and engage an interior surface of the first part. This can provide structural integrity and coupling, whilst spacing the interior surfaces to provide space for the sensor electronics 122. In this example, the supporting posts each have a recess (e.g., the posts are hollow) for receiving a protrusion on the first part (not shown). For example, the first part may include a male connector for engaging a female connector on the second part 1436 (or vice versa). In other examples, the supporting posts from the second part 1436 may be glued to the interior surface of the first part (or vice versa).

### On-Body Devices 100-15 Through 100-32

Additional examples of housing shapes are shown for the embodiments of OBDs 100-15 through 100-32 in FIGS. 1N3 through 1N-20, respectively. OBDs 100-15 through 100-32 are the same as OBD 100-1 of FIGS. 1B-1 to 1B-3, except that OBDs 100-15 through 100-32 have housings with different shapes compared to the housing 102 of FIGS. 1B-1 to 1B-3. OBDs 100-15 through 100-32 may otherwise include the same features as OBD 100-1 of FIGS. 1B-1 to 1B-3. The optional features and alternatives described in relation to OBD 100-1 may similarly be applied to OBDs 100-15 through 100-32 in a corresponding manner.

FIGS. 1N-3 through 1N-20 are bottom views showing OBDs 100-15 through 100-32 with the adhesive patch removed (for the purposes of illustration) to show the exposed base surfaces of each of OBDs 100-15 through 100-32. Although not shown, the adhesive patches for each embodiment of OBDs 100-15 through 100-32 can have a circular outer peripheral border shape like that shown in FIGS. 1B-2 and 1B-3. Patches 106 having round outer peripheral borders, particularly circular, are beneficial as they present a symmetrical boundary with no projecting edge portion (e.g., such as a sharp corner) that might be more susceptible than other edges to peeling away from the skin. However, depending on the configuration of OBD 100 and the strength of its adhesive patch 106, different shapes can be implemented for the outer periphery. For example, the adhesive patches for each embodiment of OBDs 100-15 through 100-32 can have an outer peripheral shape that matches the outer peripheral shape of the housing itself, as shown by the dashed line 106 in the example of FIG. 1N-3. In all embodiments the patch 106 can be slightly larger than the housing as shown in FIG. 1N-3 or can have an outer peripheral border that coincides with the outer peripheral border of the housing (such that they are the same size).

Each embodiment of OBDs 100-15 through 100-32 can be configured to operate with any of the sensors 160 described herein (wholly in vivo, partially in vivo, wholly ex vivo). For purpose of illustration, OBDs 100-15 through 100-32 are shown being configured to operate with partially in vivo sensors, and thus each is shown with a sensor aperture 112, though such can be omitted when configured to operate with wholly in vivo or wholly ex vivo sensors. In each embodiment of OBD 100 described herein, the sensor aperture 112 can be positioned generally centrally (e.g., as depicted in FIG. 1N-3 at both a midpoint of base surface width (if length and width are unequal then the lesser dimension of the two, which is left to right in FIG. 1N-3) and a midpoint of base surface length (the greater dimension which is top to bottom in FIG. 1N-3)), can be positioned partially off-center (e.g., as depicted in FIG. 1N-14 at a midpoint of base surface width but not a midpoint of base surface length, or vice-versa as depicted in FIG. 1N-15), or can be positioned entirely off-center (e.g., as depicted in FIG. 1N-11 at neither a midpoint of base surface length nor a midpoint of base surface width). For purposes of convenience most embodiments show the sensor aperture positioned centrally with respect to base surface length and width.

In each embodiment of OBDs 100-15 through 100-32, the housing can have a top surface that matches the shape and side of the bottom surface shown in FIGS. 1N-3 through 1N-20, similar to the embodiments shown in FIGS. 1B-2 and 1C-1. In such cases the side surface of the housings will extend generally perpendicular to the top and bottom surfaces. In other embodiments the top surface can differ in size and shape from the bottom surface, and the side surfaces can be non-perpendicular and straight (e.g., FIG. 1D) or curved (e.g., FIG. 1E).

Referring now to each of FIGS. 1N-3 through 1N-20 individually, in the embodiment of FIG. 1N-3, OBD 100-15 has a base surface 240 with an outer peripheral boundary that is partially polygonal and partially curved. A first boundary side 241 is straight along its length, and transitions to a second boundary side 242 that is both straight, along portions 243 and 244, and curved, in the portion 245 between portions 243 and 244. The curved portion 245 can have a constant radius (as shown here) or a variable radius. The boundary sides 241 and 242 together form a closed shape. The corners are sharp but can alternatively be curved or rounded. As shown here, the boundary of base surface 240 can be described as partially square (or rectangular) and partially circular. Alternatively, the boundary of base surface 240 can be described as U-shaped.

In the embodiment of FIG. 1N-4, OBD 100-16 has a base surface 246 with an outer peripheral boundary that is partially straight and partially curved. A first boundary side 247 is straight along its length, and transitions to a second boundary side 248 that is curved along its length. The boundary sides 247 and 248 together form a closed shape. The curved side 248 can have a constant radius (as shown here) or a variable radius. The corners are curved but can alternatively be sharper. As shown here, the boundary of base surface 246 can be described as semi-circular.

In the embodiment of FIG. 1N-5, OBD 100-17 has a base surface 250 with an outer peripheral boundary that is partially convex and partially concave. A first boundary side 251 is concave (indented) and curved along its length, and transitions to a second boundary side 252 that is convex and curved along its length. The boundary sides 251 and 252 together form a closed shape. The sides 251 and 252 can each have a constant radius (as shown here) or a variable radius or a combination. The corners are curved but can alternatively be sharper. As shown here, the boundary of base surface 250 can be described as circular with a single indentation.

In the embodiment of FIG. 1N-6, OBD 100-18 has a base surface 253 with an outer peripheral boundary having convex portions and concave portions. A first boundary side 254 is convex and curved along its length, and transitions to a second boundary side 255 that is concave and curved along its length, which in turn transitions to a third boundary side 256 that is convex and curved along its length, which finally transitions to a fourth boundary side 257 that is concave and curved along its length. The boundary sides 254-257 together form a closed shape. The sides 254-257 can each have a constant radius (as shown here) or a variable radius or a combination. The corners are curved but can alternatively be sharper. As shown here, the boundary of base surface 250 can be described as circular with multiple indentations.

In the embodiment of FIG. 1N-7, OBD 100-19 has a base surface 258 with an outer peripheral boundary that is lobed. A first boundary side 259 is convex and curved along its length to form a first lobe, and transitions to a second boundary side 260 that is convex and curved along its length to form a second lobe. The boundary sides 259 and 260 meet at corners that can be rounded or curved (as shown here) or can be sharp. The boundary sides 259 and 260 together form a closed shape. The curved boundary sides 259 and 260 can each have a constant radius (as shown here) or a variable radius or a combination. As shown here, the boundary of base surface 258 can be described as having two lobes. Alternatively, the boundary of base surface 258 can be described as a figure “8” shape.

In the embodiment of FIG. 1N-8, OBD 100-20 has a base surface 261 with an outer peripheral boundary that is lobed. A first boundary side 262 is convex and curved along its length to form a first lobe. The first boundary side 262 is connected to a second boundary side 263, which in turn is connected to a third boundary side 264 that connects back to the first boundary side 262 to form a closed shape. The second boundary side 263 is convex and curved along its length to form a second lobe. The third boundary side 264 is convex and curved along its length to form a third lobe. The boundary sides 262-264 meet at corners that can be rounded or curved (as shown here) or can be sharp. The boundary sides 262-264 can each have a constant radius (as shown here) or a variable radius or a combination. As shown here, the boundary of base surface 261 can be described as having three lobes. As shown here, the boundary of base surface 261 can be described as a trefoil. Alternatively, the boundary of base surface 261 can be described as petal shaped. Other embodiments can have four lobes (e.g., quatrefoil), five lobes, six lobes, seven lobes or more lobes.

In the embodiment of FIG. 1N-9, OBD 100-21 has a base surface 266 with an outer peripheral boundary side 267 that is curved along its length to form a closed shape that has a variable radius of curvature along its length and can be referred to as an ellipse.

In the embodiment of FIG. 1N-10, OBD 100-22 has a base surface 268 with an outer peripheral boundary 265-1 (solid line) with a first portion 269 that has a fixed radius of curvature along its length and a second portion that has a variable radius of curvature along its length that together form a closed shape. The first portion 269 can be described as semi-circular and the second portion 270 can be described as semi-elliptical. As shown here, the boundary of base surface 268 can be described as egg-shaped. An alternative embodiment of OBD 100-22 is shown in this figure where the boundary is indicated by dashed line 265-2. In this alternative embodiment, the boundary 265-2 of base surface 268 again has a fixed radius of curvature along the length of first portion 269, which then transitions to second portion 270 which has a variable radius of curvature along its length with a corner at the midpoint. The corner is shown here as sharp but can be rounded in other embodiments, to a degree less than that of the egg-shape boundary 265-1. As shown here, boundary 265-2 of base surface 268 can be described as a teardrop or tear-drop shaped.

In the embodiment of FIG. 1N-11, OBD 100-23 has a base surface 271 with an outer peripheral boundary that is partially convex and partially concave. A first boundary side 272 is concave (indented) and curved along its length, and transitions to a second boundary side 273 that is convex and curved along its length. The boundary sides 272 and 273 together form a closed shape. The sides 272 and 273 can each have a variable radius (as shown here) or a constant radius or a combination. The corners are curved but can alternatively be sharper. As shown here, the boundary of base surface 271 can be described as egg-shaped with a single indentation. In other embodiments two or more indentations can be present.

In the embodiment of FIG. 1N-12, OBD 100-24 has a base surface 275 with an outer peripheral boundary that is partially straight and partially curved. A first portion 277 of the boundary is curved along its length, and transitions to a second portion 278 and a third portion 279 of the boundary that are both straight and parallel. Second portion 278 and third portion 279 transition to a fourth portion 280 that is curved along its length in the same manner as portion 277 so that the portions 277-280 together form a closed symmetrical shape. The curved portions 277 and 280 can have a constant radius (as shown here) or a variable radius. As shown here, the boundary of base surface 240 can be described as partially rectangular and partially circular. Alternatively, the boundary of base surface 240 can be described as a stadium or an obround.

In the embodiment of FIG. 1N-13, OBD 100-25 has a base surface 281 with an outer peripheral boundary that is partially straight and partially curved. A first portion 282 of the boundary is curved along its length, and transitions to a second portion 283 and a third portion 284 of the boundary that are both straight and diverging away from portion 282. Second portion 283 and third portion 284 transition to a fourth portion 285 that is curved along its length with a greater radius than portion 282 so that the portions 282-285 together form a closed symmetrical shape. The curved portions 282 and 285 can have a constant radius (as shown here) or a variable radius. As shown here, the boundary of base surface 281 can be described as a tapered stadium or a tapered obround.

The shape of the base surface of OBD 100 can be polygonal. Example embodiments of polygonal base surfaces are shown in FIGS. 1N-14 though 1N-20. In each embodiment of a polygonal base surface, the corner between sides can be rounded or curved (e.g., as shown in FIGS. 1N-14 though 1N-20), or can be sharp, or a combination of both. The polygonal shapes can be symmetrical such that one side is a mirror image of an opposite side across an axis, such as axis 286 of FIG. 1N-14.

FIG. 1N-14 depicts an example embodiment of OBD 100-26 having a base surface 287 with an outer peripheral boundary that has three sides, or triangular. Here the triangle is equilateral, but can be acute, obtuse, or a right triangle in other embodiments.

FIG. 1N-15 depicts an example embodiment of OBD 100-27 having a base surface 288 with an outer peripheral boundary that has four sides, or quadrilateral. Here the quadrilateral is rectangular, but can be a square, a parallelogram, or a rhombus in other embodiments.

FIG. 1N-16 depicts an example embodiment of OBD 100-28 having a base surface 289 with an outer peripheral boundary that has five sides, or pentagonal. Here the pentagon has sides of equal length, but in other embodiments the sides can have different lengths.

FIG. 1N-17 depicts an example embodiment of OBD 100-29 having a base surface 290 with an outer peripheral boundary that has six sides, or hexagonal. Here the hexagon has sides of equal length, but in other embodiments the sides can have different lengths.

FIG. 1N-18 depicts an example embodiment of OBD 100-30 having a base surface 291 with an outer peripheral boundary that has seven sides, or heptagonal. Here the heptagon has sides of equal length, but in other embodiments the sides can have different lengths.

FIG. 1N-19 depicts an example embodiment of OBD 100-31 having a base surface 292 with an outer peripheral boundary that has eight sides, or octagonal. Here the octagon has sides of equal length, but in other embodiments the sides can have different lengths.

FIG. 1N-20 depicts an example embodiment of OBD 100-32 having a base surface 293 with an outer peripheral boundary that has twelve sides, or dodecagonal. Here the dodecagon has sides of equal length, but in other embodiments the sides can have different lengths.

These are just several examples of the many types of polygonal base surfaces that OBD 100 can have. In other embodiments, OBD 100 can have a base surface that has nine sides (nonagonal), ten sides (decagonal), 11 sides (hendecagonal), 13 sides (triskaidecagonal), 14 sides (tetradecagonal), 15 sides (pentadecagonal), 16 sides (hexadecagonal), 17 sides (heptadecagonal), 18 sides (octadecagonal), 19 sides (enneadecagonal), 20 sides (icosagonal), or more.

In other embodiments, OBD 100 can have a base surface with other shapes. Several examples include a crescent shape, a Reuleaux polygon (e.g., Reuleaux triangle or square) shape, an annular shape, and a shape formed by the intersection of two other shapes (such as a vesica piscis).

The shapes described with respect to FIGS. 1N-3 to 1N-20, and those otherwise described in this section, can each be applied to all of the other embodiments of OBD 100 described herein. The shapes described with respect to FIGS. 1N-3 to 1N-20, and those otherwise described in this section, can have interruptions in the boundary sides formed by features such as recesses, channels, grooves, textures, and/or projections that may change the shape of the boundary but do not change the characterization of the shape of the boundary (e.g., straight, rounded, etc.) along its length.

### Analyte Sensor

Sensors 160 can have various shapes, layouts, and principles of operation in the present embodiments. Described herein are just a few examples of sensors 160. Sensor 160 is configured to generate a signal indicative of an analyte level of the subject.

Sensor 160 can be a glucose sensor configured to generate a signal indicative of the glucose level of the subject. Sensor 160 can be used to monitor other analytes in addition to, or instead of, glucose. The other monitored analyte may be, for example, ammonium, asparagine, aspartate, cholesterol, choline, creatinine, diaphorase inhibitors, ethanol, glutamate, glycerol, one or more ketones, lactate, lactose, oxygen, phosphate, potassium, pyruvate, and uric acid.

Two or more analytes may be monitored by a given sensor 160 or combination of sensors 160 operating in conjunction with a single OBD 100. For example, OBD 100 can be configured to monitor glucose and one or more ketones. In another example, OBD 100 is configured to monitor glucose and lactate. In another example, OBD 100 is configured to monitor glucose, ketones, and lactate. In another example, the monitored analytes may be one or more ketones and lactate.

The term “sensor 160” collectively refers to partially implantable sensors 161 (also referred to as partially in vivo sensors or partially insertable sensors), wholly implantable sensors 162 (also referred to as wholly in vivo sensors), the wholly ex vivo sensors (can also be referred to as wholly non-implantable sensors) described herein, and otherwise within the scope of the present description. Example embodiments of partially implantable sensors 161 are described with respect to FIGS. 1O-1 to 1O-4, FIGS. 1P-1 to 1P-3, FIG. 1Q-1 and FIG. 1Q-2. Partially implantable “sensor 161” collectively refers to both planar substrate variants 161A and wire variant 161B. A partially implantable sensor 161 has a portion 120 that is configured for implantation into the body for contacting bodily fluid for the purpose of measuring one or more analytes, and a portion 118 that is configured to remain outside of the body (not inserted) for the purpose of connecting the portion 118 to the electronics of OBD 100. Example embodiments of wholly implantable (also referred to as wholly insertable) sensors 162 are described with respect to FIG. 1B-4 and FIGS. 1T-7 to 1T-11. A wholly implantable sensor 162 is configured such that its entirety is implantable within the body with no portion extending outside of the body, nor any physical (wired) connection existing between the wholly implantable sensor 162 and OBD 100 during operation. Examples of wholly ex vivo (also referred to as non-invasive or non-insertable) sensors are also described (see FIGS. 1T-12 and 1T-13). A wholly ex vivo sensor is configured such that no portion of the sensor is implanted in the body, with the entirety of the sensor remaining outside of the body while operating in conjunction with OBD 100.

As described with respect to FIG. 1B-1, sensor 161 can be configured to be inserted in the body of the subject such that a sensing region of sensor 161 is under the skin, in a subcutaneous location. More specifically, a region of sensor 161 can be placed under the skin (e.g., the sensing region that is configured to measure the analyte level). Thus, sensor 161 can be configured to extend through the skin and, in such embodiments, sensor 161 may be referred to as a transcutaneous sensor 161. Sensor 161 is configured to be in contact with bodily fluid. Sensor 161 can be configured to be in contact with interstitial fluid. In other examples, the bodily fluid may be blood from a capillary in addition to fluid from other sources. In other examples, sensor 161 can be configured to be inserted into the skin such that the sensing region resides in the epidermis and the bodily fluid is fluid from the epidermis, or inserted into the skin such that the sensing part resides in the dermis (the dermal layer) and the bodily fluid is fluid from the dermis. When configured for sensing epidermal or dermal fluid, portion 164 can be configured to be inserted into but not entirely through the skin. Sensor 161 can be of a type referred to as minimally invasive. Sensor 161 can be a microneedle or an array of microneedles. Sensor 161 can be inserted, and OBD 101 applied, by the subject or another user to the subject, for example by using an inserter or applicator.

Sensor 161 is configured to sample the analyte level of the subject and output a signal (e.g., a current or voltage) for receipt by electronics 122. Sensor 161 can be configured to sample the analyte level continuously, at random intervals, at periodic intervals (where each sampling is a discrete single sampling or a sampling for a longer duration), and/or on-demand.

Embodiments of sensor 161 will now be described with reference to variants 161A and 161B. Reference to “sensor 161” herein collectively refers to all of the embodiments of sensor 161A and 161B.

FIG. 1O-1 is a perspective view of an example embodiment of a partially implantable sensor 161A in a transverse configuration with second portion 120 extending along an axis 143 transverse to an X-Y plane occupied by first portion 118. Second portion 120 is configured to be positioned into the skin of the subject and can, in some embodiments, extend through the skin into a subcutaneous space. Second portion 120 includes a sensing region 140 that has detection capability for an analyte measured by system 10. Sensing region 140 is configured to be positioned in contact with analyte-bearing bodily fluid of the subject, such as interstitial fluid, dermal fluid, epidermal fluid, or blood.

In these embodiments, first portion 118 of sensor 161A is configured to be positioned outside of the body of the subject. First portion 118 is configured to be electrically coupled to electronics 122. First portion 118 may also be configured to be physically coupled to electronics 122.

In this manner, sensor 161A is configured to be partially implantable into the body of the subject. For example, part of sensor 161A is positioned in the body (e.g., portion 120) and part of sensor 161A is positioned out of the body (e.g., portion 118).

Sensor 161A can be fabricated in this transverse configuration of FIG. 1O-1 or can be fabricated in a continuous planar configuration and then bent to form the transverse configuration.

FIG. 1O-2 is a perspective view of another example embodiment of a partially implantable sensor 161A in a transverse configuration. Here, second portion 120 includes two sensing regions 140A and 140B. Sensing region 140A is configured to detect a first analyte (such as any of those disclosed herein) and sensing region 140B is configured to detect a second analyte (such as any of those disclosed herein) that is different from the first analyte. Here the sensing regions 140A and 140B are adjacent to each other along a length of portion 120, but in other embodiments the sensing regions 140A and 140B can be overlaid so that the working electrodes are each at or near the terminus of portion 120 in the subject's body.

FIG. 1O-3 is a cross-sectional view depicting an example embodiment of a second portion 120 of sensor 161A having a sensing region 140 with electrodes on different sides of a bendable insulative base substrate 170 in a stacked configuration. This configuration can be used with any embodiment of sensor 161A. In this embodiment, sensor 161A is an electrochemical sensor, configured to measure the level of an analyte via electrochemical oxidation/reduction reactions. Sensor 161A includes electrodes 148. In particular, sensor 161A includes at least one working electrode 168a and at least one further electrode. In this embodiment, sensor 161 includes three electrodes 148 labeled as 148a, 148b, and 148c. In this embodiment, the electrodes 148 include a working electrode 148a, a reference electrode 148b, and a counter electrode 148c. The working electrode 148a is the electrode at which the analyte (or a product which depends on the analyte level) is electrolyzed (e.g., electro-oxidized or electro-reduced). The counter electrode 148c provides an electrode paired with the working electrode, through which passes a current equal in magnitude and opposite in polarity to the current passing through the working electrode. The reference electrode 148b has a stable and known potential. In other examples, the reference electrode 148b and the counter electrode 148c are provided by the same electrode (a counter/reference electrode). Thus, there may be only two electrodes 148.

In some examples, the counter and/or the reference electrode may be omitted. For example, sensor 161A can be self-biasing (e.g., self-powered) and there may be no need for a reference electrode. In some examples, a plurality of working electrodes may be provided, such as when measuring two different analytes with the same sensor 161A.

Electrodes 148 may be made from conductive material. For example, one or more of electrodes 148 (e.g., the working electrode, the counter electrode, and/or the reference electrode) may be made from carbon, graphite, a conductive polymer, platinum, gold, palladium, iridium, graphite, or an alloy (e.g., platinum-iridium). In some examples, reference electrode and/or the counter electrode may be made from silver/silver chloride. In some examples, the working electrode and the counter electrode may be made from carbon, and the reference electrode may be made from silver/silver chloride.

Substrate 170 may be a dielectric (e.g., an electrical insulator). For example, substrate 170 may be a polymer or a plastic, such as polycarbonates, polyesters (e.g., Mylar™ and polyethylene terephthalate (PET)), polyvinyl chloride (PVC), polyurethanes, polyethers, polyamides, polyimides, or copolymers of these thermoplastics, such as PETG (glycol-modified polyethylene terephthalate). In other examples, substrate 170 may be a ceramic material, such as aluminum oxide or silicon dioxide. Substrate 170 provides a structure for sensor 161A, and also allows the various components attached to the substrate 170 to be electrically insulated from each other if desired.

Substrate 170 has a first conductive layer 171 on its first side and a second conductive layer 172 on its second side. A first insulative layer 181 covers a portion of conductive layer 171 and a second insulative layer 182 covers a portion of conductive layer 172. While depicted as extending to the terminus 141 of substrate 170, one or both of conductive layers 171 and 172 may terminate short of terminus 141. Conductive layer 172 includes a working electrode 148a having a sensing component 142 disposed on at least a portion of conductive layer 172. While a single sensing component 142 is shown, in other embodiments a plurality of spatially separated sensing components or layers may be utilized (e.g., a series of dots) on layer 172.

A conductive layer 173 is located on insulative layer 182. Conductive layer 173 is configured to include a reference electrode 148b which includes a secondary layer of conductive material 218, e.g., Ag/AgCl, disposed on a distal portion of conductive layer 173. An insulative layer 183 is located on a first surface of conductive layer 173 including reference electrode 148b. At least the ends of secondary conductive material 174 which extend along the side edges of sensor 161A are not covered by insulative layer 183 and, as such, are exposed to the environment for operation of the reference electrode 148b when in operative use. First insulative layer 181 does not extend to the terminus of conductive layer 171 leaving an exposed region of layer 171 that is configured to act as a counter electrode 148c.

As described in greater detail herein, one or more membranes may be provided about sensor 161A, e.g., as one or more of the outermost layer(s). Here, working electrode 148a has a first membrane layer 191 over sensing component 142 to modulate the rate of diffusion or flux of the analyte to the sensing component. A second membrane layer 192 is coated over the remaining surface area of second portion 120, may also be provided to serve as a biocompatible conformal coating and provide smooth edges over the entirety of the sensor. Additional membranes to accomplish other functions described herein can be included. In other embodiments, a single, homogenous membrane performing the functionality of both membranes 191 and 192 (and other functions described herein) may be coated over the entire portion 120.

Conductive layer 172 can extend between working electrode 148a and a first contact 150a on portion 118 and form an electrical connection 152 therebetween. Conductive layer 173 can extend between reference electrode 148b and a second contact 150b on portion 118 and form an electrical connection 152 therebetween. Conductive layer 173 can extend between counter electrode 148c and a third contact 150c on portion 118 and form an electrical connection 152 therebetween.

Referring to FIG. 1O-4, another embodiment of analyte sensor 161A is shown, similar to the embodiment of FIG. 1O-1. In this embodiment, the analyte sensor 161A is generally planar. In particular, the analyte sensor 161A has a height (e.g., the direction in which the second portion 120 extends) and a width (e.g., the direction in which the first portion 118 extends). The planar surface is shown in FIG. 1O-4. In this embodiment, the first portion 118 of the analyte sensor 161A extends laterally. The second portion 120 of the analyte sensor 161A extends longitudinally (e.g. in the proximal direction parallel to the central axis A). The first portion 118 extends in a plane parallel to the second portion 120. The first portion 118 can then be coupled to sensor electronics, with the first portion 118 lying in a plane generally perpendicular to the sensor electronics (e.g., perpendicular to the PCB). In this manner, the first portion 118 may be arranged upright from the sensor electronics.

Second portion 120 may have a length and a width arranged in a plane and may have a thickness perpendicular to the plane as depicted in the end on view of the sensing portion terminus of sensor 161A of FIG. 1O-5. Here, the length is into the page. The plane is indicated as 201, the width is indicated as 203 and the thickness is indicated as 205. The thickness 205 may be smaller than the width 203.

In other examples, the first portion 118 may arranged in a plane perpendicular to the second portion 120. For example, the first portion 118 may be bent or folded (e.g., about an axis in the lateral direction). In this manner, the planar surface of the first portion 118 may be perpendicular to the planar surface of the second portion 120. The first portion 118 may then extend in a plane parallel to the sensor electronics.

The analyte sensor 161A also has a thickness perpendicular to the planar surface. For example, the thickness may be perpendicular to the height and the width (e.g., into the page in FIG. 1O-4). The thickness is smaller than the height and the width. In some examples, the thickness of the first portion 118 may be different to the second portion 120. In other examples, the first portion 118 may be arranged such that the thickness of the first portion 118 is perpendicular to the thickness of the second portion 120 (e.g., the thickness of the first portion 118 extends in the longitudinal direction). For example, the first portion 118 may be folded such that the plane of first portion 118 is perpendicular to the plane of second portion 120.

In this embodiment, the analyte sensor 161A also comprises an intermediate portion 144. The intermediate portion 144 is connected to the first portion 118. The intermediate portion 144 is also connected to the second portion 120. Thus, the intermediate portion 144 connects the first portion 118 to the second portion 120. The intermediate portion 144 may allow the analyte sensor 161A to bend or fold between the first portion 118 and the second portion 120. This can allow the first portion 118 to lie in a different plane to the second portion 120.

In this embodiment, the analyte sensor 161A comprises substrate 170, similar to the embodiment of FIG. 1O-3. The substrate 146 may be flexible or rigid. The substrate 146 may be planar. The substrate 146 forms a base surface material of the analyte sensor 161A. In some examples, the substrate 146 may be a dielectric (e.g., an electrical insulator).

In this embodiment, the analyte sensor 161A is an electrochemical sensor, configured to measure the level of an analyte via electrochemical oxidation/reduction reactions. In this embodiment, the analyte sensor 161A comprises electrodes 148. In this embodiment, the analyte sensor 161A comprises three electrodes 148. In particular, the analyte sensor 161A comprises at least one working electrode and at least one further electrode. In this embodiment, the electrodes 148 comprise a working electrode, a reference electrode, and a counter electrode (not individually labeled).

In some examples, the counter and/or the reference electrode may be omitted. For example, the analyte sensor 161A may be self-biasing (e.g., self-powered) and there may be no need for a reference electrode. In some examples, a plurality of working electrodes may be provided. In some examples, multiple working electrodes may be provided, each configured to detect a different analyte. In other examples, different portions (e.g., different sensing regions) of the same working electrode may be used to detect different analytes.

In this embodiment, the electrodes 148 are arranged on the substrate 146. In particular, the electrodes 148 are arranged on the surface of the substrate 146. In other examples, the electrodes 148 may be arranged on different sides of the substrate 146. The electrodes 148 may be electrically separated from each other by the substrate 146 and/or an additional dielectric material or electrical insulator. For example, the analyte sensor 161A may have a stacked configuration with multiple layers (FIG. 1O-3). The electrodes 148 may be arranged in layers (e.g., planar layers) separated by layers of dielectric material. For example, each electrode 148 may be in a different layer. In other examples, one or more electrodes 148 may be coplanar. For example, multiple electrodes may be arranged on the same surface of the substrate 146 (e.g., on one side), separated by dielectric.

Electrodes 148 are positioned on the second portion 120. In particular, the electrodes 148 are configured to be inserted into the body when the second portion 120 is inserted into the body. For example, the electrodes 148 may be located towards the proximal end of the second portion 120 (e.g., towards the tip).

The embodiments of analyte sensor 161 comprises sensor contacts 150. In FIG. 1O-4, the analyte sensor 161A comprises three sensor contacts 150. In particular, each of the sensor contacts 150 corresponds to a particular electrode 148. In this embodiment, the sensor contacts 150 are positioned on the first portion 118. The sensor contacts 150 are electrically conductive. The sensor contacts 150 are configured to allow an electrical connection between the analyte sensor 161 and the sensor electronics. For example, the sensor contacts 150 may be configured to be in electrical contact with the electrical contacts of the sensor electronics 122.

In the embodiments of analyte sensor 161, the sensor 161 comprises connections 152. The connections 152 may be referred to as traces. The connections 152 are electrically conductive. The connections 152 are connected to the electrodes 148. The connections 152 are also connected to the sensor contacts 150. In particular, the connections 152 provide an electrical connection between the electrodes 148 and the sensor contacts 150. In this manner, an electrical signal generated at the electrodes 148 can be passed along the connections 152 to the sensor contacts 150. The signal can then be passed to the sensor electronics (e.g., to the electrical contacts of the sensor electronics 122). This signal corresponds to the measured analyte level.

In some examples, the electrodes 148, the sensor contacts 150, and/or the connections 152 may be fabricated from a conductive material, such as carbon (e.g., graphite). In other examples, the material may be a conductive polymer, platinum, gold, palladium, iridium, or an alloy (e.g., platinum-iridium). Each of the connected electrodes 148, connections 152, and sensor contacts 150 may be made from the same material.

In FIG. 1O-4, the connections 152 are arranged in the substrate 146. For example, the connections 152 may be embedded within the substrate 146. In another example, the connections 152 may be arranged on a surface of the substrate 146. In some examples, the connections 152 and the electrodes 148 may be integral. In some examples, the sensor contacts 150, the connections 152, and the electrodes 148 may be integral. For example, the electrodes 148 may each extend to the first portion 118 and thus the connection 152 and the sensor contacts 150 may be part of the electrode 148.

As already discussed, the embodiments of analyte sensor 161 may include one or more membranes. The membrane may have one or more layers. In some examples, the membrane may include a sensing layer. For example, the analyte sensor 161 may include a sensing layer. This may otherwise be referred to as a catalyst layer or an enzyme layer. The sensing layer may include a catalyst to catalyze the reaction of the analyte. For example, the catalyst may be an enzyme. Some analytes, such as oxygen, can be directly electro-oxidized or electro-reduced at the working electrode. Other analytes, such as glucose, may use a catalyst to facilitate the electro-oxidation or electro-reduction of the analyte. In this embodiment, where the analyte is glucose, the enzyme may be glucose oxidase. Glucose oxidase reacts with glucose and oxygen to produce hydrogen peroxide, which reacts at the working electrode to produce an electrical signal proportional to the glucose concentration. In other examples, other enzymes may be provided (either for monitoring glucose or for other analytes). For example, for monitoring lactate, lactate oxidase may be used as an enzyme.

The sensing layer may include an electron transfer agent to transfer electrons between the analyte and the working electrode. An electron transfer agent is a compound that carries electrons between the analyte and the working electrode. For example, the electron transfer agent may be a redox mediator. In some examples, the electrolysis may be performed without an electron transfer agent.

In some examples, the membrane may include one or more interferent layers. For example, the analyte sensor 161 may include one or more interferent layers. The interferent layer may prevent interferents or contaminants from interfering with the sensing components (e.g., electrodes 148) of the analyte sensor 161. For example, interferents may include acetaminophen (paracetamol), urate (uric acid), and ascorbate (ascorbic acid or vitamin C). Interferents may otherwise react at the electrodes, leading to a false signal.

In some examples, the membrane may include a diffusion limiting layer. For example, the analyte sensor 161 may include a diffusion limiting layer. This may otherwise be referred to as a mass transport limiting layer, flux limiting layer, or a resistance layer. This may limit diffusion of the analyte (e.g., glucose and/or oxygen) to maintain a linear signal response and improve signal stability.

In some examples, the membrane may include a biocompatible layer. For example, the analyte sensor 161 may include a biocompatible layer. In some examples, one or more membrane layers (e.g., an outer layer) is biocompatible (e.g., there is not a separate biocompatible layer). The biocompatibility allows the analyte sensor 161 to be safely positioned in the body of the user and may reduce the impact of a physiological response on operation of the analyte sensor 161.

The membrane layers described above may be provided as separate layers. For example, the analyte sensor 161 may comprise a plurality of membranes. In other examples, the one or more layers of the membrane may be combined into one layer. For example, the functionality of multiple layers described herein may be combined and provided by one or more layers. The layers may therefore be formed together as an interconnected, linked, integrated, or mixed membrane. In some examples, the one or more layers may be referred to as one or more domains.

FIG. 1P-1 is a perspective view depicting another example embodiment of analyte sensor 161, referred to as sensor 161B, which includes the same features as sensor 161A of FIGS. 1O-1 to 1O-4, except as explained below. The optional features and alternatives described in relation to the sensor 161A may be applied to sensor 161B in a corresponding manner.

In this embodiment, sensor 161B is a wire sensor with a sensing region 140. In particular, sensor 161B does not have a planar substrate (e.g., the substrate 170 of sensor 161A), but instead has a generally cylindrical shape. Sensor 161B has a transverse configuration with portion 120 at an angle with respect to portion 118. Sensing region 140 is located on end portion 120.

FIG. 1P-2 is a perspective view depicting another example embodiment of analyte sensor 161B. Here, portion 120 includes two sensing regions 140A and 140B. Sensing region 140A is configured to detect a first analyte (such as any of those disclosed herein) and sensing region 140B is configured to detect a second analyte (such as any of those disclosed herein) that is different from the first analyte. Here the sensing regions 140A and 140B are adjacent to each other along a length of the wire sensor, but in other embodiments the sensing regions 140A and 140B can be overlaid so that the working electrodes are each at or near the terminus of portion 120 of the wire sensor in the subject's body.

FIG. 1P-3 is a perspective view depicting an example embodiment of sensor 161B in greater detail. Here, sensor 161B is configured to detect or measure one analyte and includes a working electrode 168a that can have a similar function to the working electrode of sensor 161A. For example, the working electrode 148a may be made from carbon (e.g., graphite), a conductive polymer, platinum, gold, palladium, iridium, or an alloy (e.g., platinum-iridium). In some examples, the working electrode may be made from platinum or platinum-iridium.

In this embodiment, the working electrode 148a is a cylindrical wire. The working electrode 148a has a circular cross section. The working electrode 148a is a central wire. The other layers of the analyte sensor 161B are then built circumferentially around the working electrode 148a. This means that a substrate may not be required. Thus, the analyte sensor 161B has a conductive core. In other examples, a central substrate (e.g., wire) may be provided and the working electrode 148a may be arranged around the central substrate.

In this embodiment, the analyte sensor 161B comprises a dielectric 239. The dielectric 239 is a cylindrical layer around the working electrode 148a (e.g., arranged concentrically). The dielectric 239 is an electrical insulator. For example, the dielectric 239 may be a polymer or a plastic, such as a thermoplastic. For example, the dielectric 239 may be made from a polycarbonate, polyester (e.g., Mylar™ or polyethylene terephthalate (PET)), polyvinyl chloride (PVC), polyurethane (PU), polyether, polyamide, polyimide, or a copolymer, such as a copolymer of such polymers (e.g., glycol-modified polyethylene terephthalate (PETG)). For example, the dielectric 239 may polyurethane. In other examples, the dielectric 239 may be a ceramic material, such as aluminum oxide or silicon dioxide.

In this embodiment, the analyte sensor 161B comprises a counter/reference electrode 148b. In particular, there is one electrode that performs the function of both the counter and reference electrodes. The counter/reference electrode 148b is a cylindrical layer around the dielectric 239. Thus, the electrodes 148 may be in concentric layers (separated by dielectric 239). The counter/reference electrode 148b may be made from conductive material. In some examples, the counter/reference electrode 148b may be made from silver/silver chloride.

In this embodiment, the analyte sensor 161B comprises one or more regions in which the counter/reference electrode 148b and the dielectric 239 is removed. In this manner, the analyte sensor 161B comprises a region at which the working electrode 148a is exposed at the proximal end. For example, the dielectric 239 may be stripped or skived to expose the working electrode 148a. The exposed section may act as the working electrode 148a. This forms the sensing region of the analyte sensor 161B (or the active area of the working electrode 148a). This is at the second portion 220 which is configured to be inserted into the body. By removing the dielectric 239, the area of the exposed working electrode 148a can be controlled. The sensing layer of the membrane may be applied over the sensing region of the analyte sensor 161B. The other layers (e.g., interferent layer, diffusion limiting layer, and/or biocompatible layer) may be applied over the second portion 220 (e.g., the entire portion inserted into the body 5), for example rather than just over the active area of the working electrode 148a.

At the proximal tip of the analyte sensor 161B (e.g., at the second portion 220), the dielectric 239 may be arranged over the working electrode 148a. For example, this may protect the working electrode 148a and provide structural integrity.

The working electrode 148a may similarly be exposed at the first portion 218 for electrically coupling to the sensor electronics (e.g., the sensor electronics 122). For example, one electrical contact of the sensor electronics may be in contact with the working electrode 148a, and another electrical contact may be in contact with the counter/reference electrode 148b. The analyte sensor 161B may thus not have separate connections and sensor contacts (such as the connections 152 and sensor contacts 150 of the analyte sensor 161A). The first portion 218 may be arranged out of the body 5, as described herein.

Although the first portion 218 and the second portion 220 are shown along one axis in FIG. 1P-3, the analyte sensor 161B may be bent or folded such that the second portion 220 is perpendicular to the first portion 218 as shown in FIG. 1P-1.

In the embodiments of sensors 161A and 161B having multiple (e.g., two, three, or more) sensing regions (e.g., 140A and 140B), each sensing region can have its own discrete electrodes such that none are shared. Alternatively, one or more electrodes can be shared between the sensing regions, and one or more electrodes can be specialized for the analyte particular to that sensing region 140 (and not shared). For example, a single reference electrode can be shared between sensing regions 140A and 140B, and sensing region 140A can have its own working electrode, while sensing region 140B can have its own separate working electrode. In embodiments with a discrete counter electrode, the single counter electrode can be shared between sensing regions 140A and 140B or each region 140A and 140B can have its own counter electrode.

Additional example embodiments having sensor 161A are now described. Referring to FIG. 1Q-1, another embodiment of analyte sensor 161A is shown. The analyte sensor 161A may be provided in any embodiment of OBD 100 configured for a partially implantable sensor described herein, such as OBD 100-13 shown in FIG. 1N-1. In other examples, the analyte sensor 161A may be provided in an OBD with various modifications such as described in relation to OBD 100-1 of FIGS. 1B-1 to 1B-3 or other embodiments.

The analyte sensor 161A is the same as the analyte sensor 161A of FIGS. 1O-1 to 1O-4, and includes the same features, except as explained below. The optional features and alternatives described in relation to analyte sensor 161A of FIGS. 1O-1 to 1O-4 may similarly be applied to the analyte sensor here in a corresponding manner.

Sensor 161A has a planar shape with first portion 118 and second portion 120. The first portion 118 extends with a length generally perpendicularly to the second portion 120 in a similar manner to the analyte sensor 161A of FIGS. 1O-1 to 1O-4. An intermediate portion between the first portion 118 and the second portion 120 comprises a bend and a distally extending portion. In some examples, the distally extending portion may be omitted. The first portion 118 has a length and a width arranged in a plane (providing a side surface of the analyte sensor 161A), and a thickness perpendicular to the plane. The thickness is smaller than the length and the width. For example, the first portion 118 lies in a vertical plane. This provides the sensor contacts in a lateral direction.

Referring to FIG. 1Q-2, another embodiment of analyte sensor 161A is shown. The analyte sensor 161A may be provided in any embodiment of OBD 100 configured for a partially implantable sensor described herein, such as OBD 100-14 shown in FIG. 1N-2. In other examples, the analyte sensor 161A may be provided in an OBD with various modifications such as described in relation to OBD 100-1 of FIGS. 1B-1 to 1B-3 or other embodiments.

The analyte sensor 161A is the same as the analyte sensor 161A of FIGS. 1O-1 to 1O-4, and includes the same features, except as explained below. The optional features and alternatives described in relation to analyte sensor 161A of FIGS. 1O-1 to 1O-4 may similarly be applied to the analyte sensor here in a corresponding manner.

The analyte sensor 161A comprises a first portion 118 and a second portion 120. The first portion 118 extends with a length generally perpendicularly to the second portion 120. An intermediate portion between the first portion 118 and the second portion 120 comprises a bend and a distally extending portion. In some examples, the distally extending portion may be omitted. The first portion 118 has a length and a width arranged in a plane (providing a side surface of the analyte sensor 161A), where the plane is arranged perpendicular to the length of the second portion 120. In this manner, the plane of the analyte sensor 161A is bent. The first portion 118 has a thickness perpendicular to the plane, and the thickness is in an axis parallel to the length of the second portion 120. The thickness is smaller than the length and the width. For example, the first portion 118 lies in a horizontal plane. This provides the sensor contacts in a distal direction (or a proximal direction in other examples). In particular, three sensor contacts are shown on the distal side of the first portion 118 (e.g., for three electrodes), but other numbers of contacts may be provided in other examples.

### Sensor Electronics

Referring to FIGS. 1R-1 and 1R-2, block diagrams of sensor electronics 122 are shown according to an embodiment. The sensor electronics 122 may be provided in all embodiments of OBD 100 described herein with various modifications such as described in relation to OBD 100-1 of FIGS. 1B-1 to 1B-3 and OBD 100-1′ of FIG. 1B-4 or other embodiments.

For purpose of illustration and not limitation, reference is made to the example embodiments of OBD 100 shown in FIGS. 1R-1 and 1R-2 as for use with the disclosed subject matter. OBD 100 can be designed to be power-efficient, low-cost, and possibly disposable. FIG. 1R-1 is a block diagram depicting an example embodiment of an OBD 100 configured for operation with sensor 160, which may be a partially implantable sensor 161 or a wholly ex vivo sensor. FIG. 1R-2 is a block diagram depicting an example embodiment of an OBD 100 configured for operation with a wholly implantable sensor 162. In these embodiments, like components are referred to with like reference numerals.

OBD 100 includes electronics 122 that interface with sensor 160, perform functions of the system, process measurement data, and perform wireless communication tasks. Operation of OBD 100 can include state management of OBD 100 (e.g., control of operating states such as awake, partially awake, sleep states), control of sensor 160, power source life assessment (e.g., measuring power source voltage and prediction of power source life), alarm or alert generation and management, and/or authentication of receiver units 20 (e.g., by verification of a unique ID associated with sensor 160). Wireless communication tasks can include controlling a state of a transceiver, wireless communication according to a custom wireless protocol or a standardized protocol (e.g., open or proprietary), encryption and decryption of data, encoding and decoding of data, and/or error correction.

Electronics 122 include integrated circuitry (IC) hardware such as one or more semiconductor chips. The semiconductor chip or chips of electronics 122 can be embodied as or with a system-on-chip (SoC) topology, a microcontroller unit (MCU), programmable gate array or field-programmable gate array (PGA, FPGA), and/or an application-specific integrated circuit (ASIC). Electronics 122 can include peripherals, input/output (I/O) ports and/or the like. Electronics 122 can include discrete active and/or passive components of various forms distributed on one or more printed circuit boards (PCBs) with the IC hardware.

As shown in FIGS. 1R-1 and 1R-2, OBD electronics 122 include processing electronics 315. Processing electronics 315 can include a general-purpose processing unit or processor (e.g., a central processing unit according to a Complex Instruction Set Computer (CISC) or Reduced Instruction Set Computer (RISC) architecture), a PGA or FPGA, a controller, a microcontroller, and/or a custom processing architecture. Processing electronics 315 are centralized or distributed within electronics 122. For example, processing electronics 315 can be centralized as a module within a single chip, distributed throughout a single chip, or distributed in one or more modules within a first ASIC chip and/or a second wireless communication electronics chip. The processing capability of processor electronics 315 can be implemented in the various IC forms described herein using hardware, firmware, software, or a suitable combination of hardware, firmware, and software.

As shown in FIGS. 1R-1 and 1R-2, electronics 122 include memory 317 in communication with processing electronics over communication interface 304. Memory 317 that is non-transitory such as volatile and/or non-volatile memory for storing instructions, information, and collected data. Instructions are stored in memory 317 in the form of software and/or firmware that is executable by processing electronics 315 in its locations throughout electronics 122. Instructions having different functions can be performed in the locations within electronics 122 most conducive to those functions. These instructions can be embedded, installed, or both embedded and installed in the electronics. Many types of memory can be used in OBD 100 including, but not limited to random access memory (RAM) (e.g., Dynamic RAM (DRAM), Static RAM (SRAM), Synchronous Dynamic RAM (SDRAM), Double Data Rate SDRAM (DDR SDRAM), Magnetoresistive RAM (MRAM), Resistive RAM (RRAM)), read only memory (ROM) (e.g., flash memory, Programmable ROM (PROM), Erasable PROM (EPROM), Electrically Erasable PROM (EEPROM)), variants thereof (e.g., virtual memory, register memory, cache memory), and/or others. Memory 317 can be in the form of one or more discrete memory chips, can be distributed throughout the semiconductor chips of OBD 100, such as within a processor chip and within a wireless communications chip, or can be both in the form of one or more discrete chips and distributed throughout one or more chips.

As shown in FIGS. 1R-1 and 1R-2, electronics 122 include wireless communication electronics 312 in communication with processing electronics 315 and memory 317 over communication interface 304. Wireless communication electronics 312 include hardware and software for performing communications with the various types of receiver units 20. The embodiments of OBD 100 can communicate with one receiver unit 20 during a single wear duration of the OBD 100, or can communicate with multiple receiver units 20 and secondary receiver units 30 during a single wear duration of the OBD 100, such as the following five embodiments: a smart-phone and a smart-watch; multiple smart-phones; a smart-phone and a dedicated receiver unit; a dedicated receiver unit and a smart-watch; or a smart-phone, a smart-watch, and a dedicated receiver unit. In these embodiments, the receiver units 20 and 30 can all be authenticated or in an authenticated state at the same time, and thus eligible to receive the same or similar communications from OBD 100 at the same time or at adjacent correlated times (e.g., with a minute or a few minutes of each other). Processing electronics 315, in whole or in part, can be integrated within wireless communication electronics 312 for efficient handling of communications tasks. In all of the embodiments herein, wireless communication electronics 312 are configured to communicate with receiver units 20 according to a Bluetooth Low Energy protocol. In all of the embodiments herein, wireless communication electronics 312 can also be configured to communicate with receiver units 20 according to a Near-Field Communication (“NFC”) protocol. In all of the embodiments herein, wireless communication electronics 312 can be configured to communicate with receiver units 20 according to two or more communication protocols, such as BLE and NFC. However, wireless communication electronics 312 can have the hardware and software to perform wireless communications with receiver units 20 in accordance with the communication protocols with which OBD 100 is configured to operate. These communication protocols (some referenced both here and elsewhere herein) include but are not limited to: Bluetooth; BLE; NFC; RFID; ultra-wideband (UWB); mobile protocols (e.g., Fifth Generation (5G) technology, Long Term Evolution (LTE), Global System for Mobile Communications (GSM), Code Division Multiple Access (CDMA)); Z-Wave; a personal area network according to Institute of Electrical and Electronics Engineers (IEEE) 802.15 protocols (e.g., Zigbee), Adaptive Network Topology (ANT), or IPv6 over Low-Power Wireless Personal Area Networks (6LoWPAN); IEEE 802.11 protocols (e.g., 802.11a, 802.11b, 802.11g, 802.11n (aka Wi-Fi 4), 802.11ac (aka Wi-Fi 5), 802.11ah (Wi-Fi HaLow), 802.11ax (aka Wi-Fi 6)); infrared communications according to the Infrared Data Association standards (IrDA); Light Fidelity (Li-Fi); low-power wide-area network (LPWAN) protocols such as LoRa (Long Range); and wireless communication protocols that are proprietary, customized, and not subject to standardization by an international, national, nor regional standard setting body.

Communication electronics 312 can include communication circuitry for each protocol with which OBD 100 operates. Shown here are an antenna 314 communicatively coupled with a transceiver 316. Transceiver 316 has both transmit and receive hardware and capability. Transceiver 316 can further include transmit and receiver amplification circuitry. The communication circuitry has the capability to support all the requisite communication protocols for the implementation of system 10 and can include more than one antenna and transceiver if necessary. For brevity, only a single set of the communication circuitry is illustrated in this embodiment. The transceiver 316 can be implemented as a single component or as a separate transmitter and a separate receiver, collectively referred to herein as the transceiver. The one or more transmit amplifiers can be selectively connected between an output of the transmitter of the transceiver 316 and the antenna 314. The one or more receive amplifiers are configured to be selectively connected between the antenna 314 and an input of the receiver of the transceiver 316.

As shown in FIGS. 1R-1 and 1R-2, OBD 100 includes sensor interface electronics 311 in communication with processing electronics 315, memory 317, and communication electronics 312 over interface 304. Sensor interface electronics 311 interface with sensor 160 in its various configurations. In the embodiment of FIG. 1R-1, sensor interface electronics 311 interface with partially implantable sensor 161 and include contacts for electrically contacting contacts 169 of sensor 161. In a similar embodiment, sensor interface electronics 311 interface with an ex vivo sensor.

Sensor interface electronics 311 can include analog front end (AFE) circuitry for generating and applying a bias current or voltage (e.g., a poise voltage) to sensor 161. Sensor 161 can generate an analog voltage or current signal corresponding to the level of analyte (e.g., glucose) in the subject's bodily fluid. AFE circuitry can receive the generated analog signal indicative of the sensed analyte level, optionally precondition the signal, convert the analog signal to a digital data form indicative of the sensed analyte level using an analog-to-digital converter, and output it to processing electronics 315 for further processing and buffering (e.g., conversion from a raw form into a form more readily representative of the concentration value of the analyte (e.g., in units of mg/dL or mmol/L)). For example, the digital analyte data can be algorithmically adjusted to account for known or estimated offsets resulting from use of sensor 160 (e.g., calibration processing, sensor shelf duration compensation, sensor wear duration compensation, or other sensor attenuation compensation). Processing electronics 315 can be partially integrated within sensor interface electronics 311 for these and other purposes. The raw or processed analyte data can be output to wireless communication electronics 312 to be transmitted to a receiver unit 20. In other examples, the algorithmic adjustment is not performed by OBD 100 and may instead be performed by receiver units 20. In an embodiment the sensor interface electronics 311 are present on a first ASIC semiconductor chip and the wireless communication electronics 312 are present on a second semiconductor chip, with the processing electronics 315 and memory 317 present on either chip or distributed therebetween. Examples of these configurations are shown and described with respect to FIGS. 1R-3 through 1R-5.

In the embodiment of FIG. 1R-2, sensor interface electronics 311 interface with a wholly implantable sensor 162 and are configured with wireless communication electronics similar to electronics 312. Sensor interface electronics 311 receive analyte measurement data wirelessly from wholly implantable sensor 162. Sensor interface electronics 311 can also transmit wirelessly to wholly implantable sensor 162. Sensor interface electronics 311 receive analyte measurement data wirelessly from wholly implantable sensor 162. Sensor interface electronics 311 can also transmit wirelessly to wholly implantable sensor 162, for example with a command to respond with analyte measurement data or report other sensor information such as a unique identifier, the existence of a fault, power supply status, and the like.

Sensor interface electronics 311 can include a transceiver 326 and antenna 324 configured to operate according to the protocol implemented by sensor 162. Sensor interface electronics 311 in this embodiment can be implemented discretely from wireless communications electronics 312 as shown here or can be part of wireless communication electronics 312 such that electronics 312 is configured to wirelessly communicate with both wholly implanted sensor 162 and one or more receiver units 20.

OBD 100 and sensor 162 can wirelessly communicate according to various protocols. In the embodiments herein, OBD 100 and sensor 162 can be configured to wirelessly communicate by the transmission of a communication from OBD 100 to sensor 162 according to a Near Field Communication (NFC) protocol. Sensor 162 can respond passively by harvesting the power from the received NFC signal. Sensor 162 can respond actively as well, or alternatively, in the embodiments herein. In the embodiments herein, OBD 100 and sensor 162 can be configured to wirelessly communicate by a Bluetooth or Bluetooth Low Energy (BLE) protocol. In the embodiments herein, OBD 100 and sensor 162 can be configured to communicate using multiple wireless protocols, such as BLE and NFC, or others. Sensor interface electronics 311 in FIG. 1R-2 can perform wireless communications in accordance with the communication protocols with which sensor 162 is configured to communicate. These communication protocols (some referenced both here and elsewhere herein) include but are not limited to: Bluetooth; Bluetooth Low-Energy (“BLE”); Near-Field Communication (“NFC”); RFID; ultra-wideband (UWB); mobile protocols (e.g., Fifth Generation (5G) technology, Long Term Evolution (LTE), Global System for Mobile Communications (GSM), Code Division Multiple Access (CDMA)); Z-Wave; a personal area network according to Institute of Electrical and Electronics Engineers (IEEE) 802.15 protocols (e.g., Zigbee), Adaptive Network Topology (ANT), or IPv6 over Low-Power Wireless Personal Area Networks (6LoWPAN); IEEE 802.11 protocols (e.g., 802.11a, 802.11b, 802.11g, 802.11n (aka Wi-Fi 4), 802.11ac (aka Wi-Fi 5), 802.11ah (Wi-Fi HaLow), 802.11ax (aka Wi-Fi 6)); infrared communications according to the Infrared Data Association standards (IrDA); Light Fidelity (Li-Fi); low-power wide-area network (LPWAN) protocols such as LoRa (Long Range); and wireless communication protocols that are proprietary, customized, and not subject to standardization by an international, national, nor regional standard setting body.

OBD 100 includes a power source 320. Power source 320 can be a battery. Power source 320 supplies electrical voltage and current to all components of electronics 122 by way of a power connection 322 that can include a power bus and PCB power and ground planes. Power source 320 can be accompanied by power management circuitry for managing the connection and disconnection of the power source from the electronics 122 and/or for boosting voltage (e.g., with a charge pump). In some embodiments power source 320 is or includes a capacitive energy storage element that can be wirelessly charged with NFC transmissions (or other inductive coupling). Also, OBD 100 can be configured without wide-area network communication capability.

OBD 100 includes one or more clocks 319. Clocks 319 can include circuitry to produce one or more clock signals to act as timing references for electronics 122. Clocks 319 can be communicatively coupled with processor electronics 315, sensor interface electronics 311, wireless communication electronics 312, and other components via interface 304. Clocks 319 can be centralized in OBD 100 or distributed in various locations to serve the circuitry that requires each timing reference. The clock signals can have different frequencies from each other. The clock signals can be used, among other things, to wake OBD 100 from a sleep state and to synchronize the timing of advertising or connection events and for entering a sleep state between the advertising or connection events. The clock signals can determine when OBD 100 should wake up next after processing the advertising or connection events before going to sleep. OBD 100 can then set an event to wake up in time for the next advertising or connection events.

Communication interface 304 can be electrical traces or connections that communicatively couple two or more entities together. Interface 304 is distributed throughout OBD 100 to permit communication between all components requiring interaction. Interface 304 can include a main communication bus and one or more I/P ports. Interface 304 can be within a chip, external to a chip (e.g., on a PCB), or both.

Additionally, processing electronics 315 can execute a startup module stored in memory 317. The startup module can include program instructions that, when executed, are utilized to control circuitry within OBD 100, such as memory 317, sensor interface electronics 311, wireless communication electronics 312, and the like. Processing electronics 315 can execute an operating system module stored in memory 317. The operating system module supports the applications or other individual functions that run within OBD 100. Processing electronics 315 and wireless communication electronics 312 can be configured to execute operations according to a protocol stack, which can include a controller and a host, each containing various communication layers. The protocol stack can include or embody the operations for OBD 100 to communicate with other devices using one or more communication protocols.

As explained herein, while the communication electronics 312 is initialized, the transmitter of transceiver 316 can transmit advertisement notices (e.g., communication packets including at least a payload including information to facilitate the initiation of discovery and a communication session with another device) arranged in complexes, followed by sleep states in accordance with an advertisement interval. The receiver of the transceiver 316 can monitor for communications pertaining to formation of a connection (e.g., a Bluetooth or BLE scan request or scan response), during a receive window. Connection requests can be sent in response to advertisement notices or independently by other devices. The connection monitoring operation, during an individual receive window, can be performed during the same period of time as transmission of the advertisement notices over corresponding advertisement channels. Optionally, the receive window and connection monitoring operation can continue after completion of transmission of the advertisement notices. Hence, the connection monitoring operation and receive window can temporarily align with the complex of advertisement notices or extend beyond the complex of advertisement notices.

As embodied herein a first layer of security for communications between OBD 100 and other devices (120, 162) can be established based on security protocols specified by and integrated in the communication protocols used for the communication (e.g., BLE security protocols, Wi-Fi security protocols). An additional, or alternative, layer of security can be based on communication protocols that necessitate very close positioning (within 6 inches or 15 cm) of communicating devices as with NFC, as such require the communicating devices to be very close to each other which makes it practically difficult to breach data security. Furthermore, certain packets and/or certain data included within packets can be encrypted while other packets and/or data within packets is otherwise encrypted or not encrypted. As an example, connection data and/or connection packets devoted to establishing communication connections between devices can be largely unencrypted (sensitive analyte data can be encrypted, even if included in a connection packet) in order to facilitate discovery by other devices.

Additionally or alternatively, another layer of security can be based on use, by OBD 100 and other devices in communication, of application layer encryption using one or more block ciphers to establish mutual authentication and encryption of other devices in system 10. The use of an encryption design implemented in the application layer has several benefits. One benefit of this approach is that in certain embodiments the user can complete the pairing of OBD 100 and another device with minimal interaction, e.g., using only a passive or active NFC data transfer operation (sometimes referred to as an NFC scan) and without requiring additional input, such as entering a security pin or confirming pairing.

Referring still to FIG. 1R-1, OBD 100 can be configured to dynamically generate authentication and encryption keys using the data retained within memory 317. Memory 317 can also have a set of valid authentication and encryption keys stored thereon to use with particular classes of devices. OBD 100 can be further configured to perform authentication procedures with other devices (e.g., handshake, mutual authentication, etc.) using received data and apply the generated key to sensitive data prior to transmitting the sensitive data, such as sending the sensitive data to the remote server 150 via communication electronics 312. The generated key can be unique to OBD 100 or sensor 160, unique to a pair of devices (e.g., unique to a particular pairing of OBD 100 and a dedicated data receiving device 120), unique to a communication session between OBD 100 and other device, unique to a message sent during a communication session, or unique to a block of data contained within a message.

OBD 100 can be implemented with or without user attachable or detachable pieces. For example, OBD 100 can be implemented in a monolithic manner from the wearer's perspective, e.g., as a single-piece OBD, where no portion of OBD 100 (except, e.g., the sensor 161 or 163) is required to be connected to another portion in order to achieve functionality on the body. Alternatively, OBD 100 can be implemented in a multi-piece (e.g., two piece) manner from the user's perspective, where two or more portions of OBD 100 are connected together by the user to achieve functionality. For example, OBD 100 can be implemented with first piece including a receptacle and adhesive patch that is first applied to the wearer's body with a partially implantable sensor 161, and then a second piece is attached to the receptacle where the second piece includes OBD electronics 122 and a housing, and upon attachment the sensor 161 is connected to the electronics 122. Alternatively, the first piece can have sensor 161 and sensor interface electronics 311 and the second piece can have the remaining electronics 122 that are connected to the sensor 161 and sensor interface electronics.

OBD 100 can, in some embodiments, be configured to include drug delivery capability. OBD 100 can be configured as an on-body injector (e.g., a pump) attachable to the subject's body and capable of automatically delivering an injection or infusion of a fixed or user-selected dose of a drug over a controlled or selected period of time. In such embodiments, OBD 100 can include a primary container for storing a drug or medicament, a drive mechanism configured to drive or permit the release of a plunger to discharge the drug from the primary container, a trocar (e.g., a solid core needle), a flexible cannula disposed around the trocar, an insertion mechanism configured to insert the trocar and/or flexible cannula into the subject and optionally retract the trocar leaving the flexible cannula in the subject, a fluid pathway connector configured to establish fluid communication between the primary container and the flexible cannula upon device activation, and an actuator (e.g., a user displaceable button) configured to activate the device. As embodied herein, the on-body injector can be pre-filled and/or pre-loaded. OBD 100 can receive instructions from a receiver unit 20 that informs as to the amount of drug to be injected or infused and when. Alternatively, OBD 100 can include a flexible cannula for drug infusion connected by tubing to a receiver unit 20 configured as a drug delivery device.

The sensor electronics 122 can include an activation switch. The activation switch is configured to cause the sensor electronics 122 to switch from a low power mode to an active mode, or from a relatively inactive mode to an active mode. In particular, the activation switch causes the power source 320 to supply power to the sensor electronics 122 as described above. In some examples, the activation switch may be a physical switch, an electrical switch, or a magnetic switch. An example of a magnetic switch is a switch that responds differently to the presence or absence of a magnetic field with a change in its analog or digital output signal, such as a Hall sensor. The activation switch can be activated after insertion of the analyte sensor onto the body. For example, the activation switch may be activated by the user (e.g., manually, or by using an inserter). In other examples, an activation switch is not provided.

In some examples, the sensor electronics 122 may be implemented on one or more printed circuit boards (PCBs). The PCB may be arranged in an orientation parallel to the base surface of the housing. Electronic components may be provided on the upper surface and/or the lower surface of the PCB. The housing may provide a space around the PCB to house the components. In other examples, the housing may be molded over the PCB without any space around the components.

FIG. 1R-3 is a top down view of an example embodiment of an upper surface 327-1 of a PCB 329-1 of an on-body device configured for operation with a partially implantable sensor 161, with various components of sensor electronics 122 electrically connected thereto. In this embodiment, the majority of electrical components are electrically connected to upper surface 327-1, including sensor interface electronics 311 (e.g., implemented on a semiconductor chip), wireless communication electronics 312 and memory 317 integrated together in a single semiconductor chip 331, processing electronics 315 and memory 317 integrated together in a single semiconductor chip 332, first antenna 314-1, second antenna 314-2, clock 319, power source 320 configured here as a coin cell battery, and an activation switch 321. In this embodiment, first antenna 314-1 is configured for communication according to a Bluetooth or Bluetooth Low Energy (BLE) wireless communication protocol. Second antenna 314-2 is configured for communication according to a Near Field Communication (NFC) wireless communication protocol. Each antenna 314-1 and 314-2 can, independently of the other, be surface printed on PCB 329-1, or can be a discrete component mounted and electrically coupled to PCB 329-1. In some embodiments antennas 314-1 and 314-2 are integrated together, such as a single surface printed antenna capable of communicating according to both protocols. Either of antennas 314-1 or 314-2 can be omitted if the implementation of on-body device 100 does not utilize Bluetooth, BLE, or NFC. Activation switch 321 is configured as a magnetic switch in this embodiment, which is configured to activate at least a portion of sensor electronics 122 when exposed to a magnetic field, such as one emanating from a magnet located in an inserter or packaging (e.g., a container) for a component (such as a sensor connector or housing 102) of on-body device 100. FIG. 1R-4 shows another example embodiment where upper surface 327-2 of PCB 329-1 includes wireless communication electronics 312, processing electronics 315, and memory 317 integrated together in a single semiconductor chip 334.

FIG. 1R-5 is a bottom up view of an example embodiment of a lower surface 328 of PCB 329-1, which can be on the same PCB as either upper surface 327-1 or 327-2. Multiple passive components (e.g., resistors and capacitors) are mounted to lower surface 328. Lower surface 328 also has a sensor contact region, which can include metallic contacts for electrically connected sensor electronics 122 to contacts on sensor 161 or to a contacting element (e.g., a receptacle for contact portion 118, a polymeric seal with contact portions, and others) that bridges contact with the actual contacts on sensor 161. Any of the elements shown on upper surfaces 327-1 and/or 327-2 can be mounted on lower surface 328, and any of those on lower surface 328 can located on either of upper surfaces 327-1 or 327-2 as desired for the implementation. PCB 329-1 includes numerous electrical connections (e.g., 304) between the various sensor electronics that are not shown for clarity.

FIG. 1R-6 is a side view depicting an example embodiment of PCB 329-1 with sensor electronics 122 arranged as described with respect to upper surface 327 (either 327-1 or 327-2) and lower surface 328. An aperture 343 to accommodate sharp and/or sensor passage or a sharp and/or sensor passageway extends through PCB 329-1 and is shown in dashed line. As can be seen here, power source 320 has a plane 335-1 extending along its length and generally central to its height that is offset to a plane 335-2 of PCB 329-1 extending along its length and generally central to its height.

FIG. 1R-7 is a top down view of another example embodiment of an upper surface 327 of a PCB 329-2 of an on-body device, with various components of sensor electronics 122 electrically connected thereto. FIG. 1R-8 is a bottom up view of another example embodiment of a lower surface 328 of a PCB 329-2 of an on-body device, with various components of sensor electronics 122 electrically connected thereto. This embodiment of PCB 329-2 can be used with all embodiments of sensor electronics 122 described herein, such as with the various chip configurations. In this embodiment, PCB 329-2 has a partially circular shape, which may be referred to as having an open lateral region. PCB 329-2 may also be referred to as semi-circular, semi-elliptical, C-shaped, or U-shaped. Power source 320, configured here as a coin cell battery, is positioned within the open lateral region and electrically connected to PCB 329-2 with connectors 336-1 and 336-2, which also provide mechanical support to power source 320. A side of view of this embodiment is shown in FIG. 1R-9. Here it can be seen that power source 320 is electrically connected to PCB 329-2 and is held adjacent to PCB 329-2 such that power source 320 and PCB 329-2 are both in a same plane 335-1,2. The plane 325-1,2 can be viewed as extending along the lengths of power source 320 and PCB 329-2 generally central to their widths. This configuration allows a significant height reduction to sensor electronics 122 as the power source height and PCB heights can overlap as opposed to being additive.

Sensor electronics 122 can include one or more temperature sensors 340 configured to measure a temperature in proximity of the sensor, which can be indicative of the temperature of the ambient environment or the body temperature of the wearer. A temperature sensor 340 can be integrated within processing electronics 315 and/or wireless communication electronics 312. A temperature sensor can also, or alternatively, be a discrete component mounted on PCB 329. A temperature sensor can also, or alternatively, be located elsewhere, such as on an exterior surface of on-body device housing 102. FIG. 1R-10 is a bottom up view of a base surface 104 of housing 102 of on-body device 100 with a temperature sensor 340 exposed thereon, and FIG. 1R-11 is a cross-sectional side view of the on-body device 100. A flexible substrate 339 is connected to PCB 329 at connection junction 341 and extends to and through a lower portion of housing 102 at opening 340. A sensing portion 338 extends outside of housing 102 and is exposed to the environment where it can measure temperature. The sensing portion 340 can change its electrical response in accordance with changing external temperature and can be electrically connected to circuitry on PCB 329 that can interface with temperature sensor 340 and sensing portion 338 to collect a measurement of the external environment that is indicative of the temperature in that environment. Sensing portion 338 can be set within a shallow recess on base surface 104 and covered with adhesive patch 106. Alternatively, patch 106 can have an opening that aligns over portion 338 so that it is not covered with patch 106 while being worn by the user.

Sensor electronics 122 can include one or more antennas 314 located off of PCB 329. FIG. 1R-11 is a cross-sectional side view of on-body device 100 with an antenna 314 mounted on housing 102 and electrically connected to wireless communications electronics 312 on PCB 329. Antenna 314 can be adhesively bonded, welded, joined, or otherwise attached to an interior surface of housing 102 or other internal structure of on-body device 100. Antenna 314 is electrically connected to PCB 329 by connection 342, which can be a wire or ribbon connector, and in turn connected to wireless communication electronics 312 either directly or indirectly through other components of PCB 329. Antenna 314 is shown on the upper interior surface of housing 102 but can be positioned elsewhere, such as a side interior surface or lower surface of housing 102 or other internal structure. Antenna 314 need not be coupled directly to housing 102 but can be coupled indirectly, such as through a liner.

While communication with the user is readily performed with the receiver unit, in some examples the OBD can include a user interface for communication to and from the user. The user interface can be a display, such as a touch screen display, that can both output information and accept input from the user. The user interface can be one or more optical indicators, such as light emitting diodes. The user interface can include one or more buttons, switches, or actuators that can be activated by the user to provide input to the OBD. In such cases where the OBD includes a user interface then the OBD can be used independently in the system without a receiver unit.

### Receiver Electronics

FIG. 1R-13 is a block diagram of receiver electronics 350 are shown according to an embodiment. Receiver electronics 350 can be configured for use with any of the receiver unit 20 and/or 30 embodiments disclosed herein, including but not limited to: a mobile device such as a mobile phone (e.g., a smartphone) and/or a wearable mobile device such as a smartwatch; a computing device, such as a computer (e.g., a tablet computer, a laptop computer, or a desktop computer) or a processing unit within a vehicle (e.g., a car or a motorcycle); a dedicated reader device for analyte monitoring; and/or a drug delivery device, such as an insulin pump or an insulin pen.

Receiver electronics 350 include integrated circuitry (IC) hardware such as one or more semiconductor chips. The semiconductor chip or chips of electronics 350 can be embodied as or with a system-on-chip (SoC) topology, a microcontroller unit (MCU), programmable gate array or field-programmable gate array (PGA, FPGA), and/or an application-specific integrated circuit (ASIC). Electronics 350 can include peripherals, input/output (I/O) ports and/or the like. Electronics 350 can include discrete active and/or passive components of various forms distributed on one or more printed circuit boards (PCBs) with the IC hardware.

As shown in FIG. 1R-13, receiver electronics 350 include processing electronics 351. Processing electronics 351 can include a general-purpose processing unit or processor (e.g., a central processing unit according to a Complex Instruction Set Computer (CISC) or Reduced Instruction Set Computer (RISC) architecture), a PGA or FPGA, a controller, a microcontroller, and/or a custom processing architecture. Processing electronics 351 are centralized or distributed within electronics 350. The processing capability of processor electronics 351 can be implemented in the various IC forms described herein using hardware, firmware, software, or a suitable combination of hardware, firmware, and software.

Receiver electronics 350 include memory 352 in communication with processing electronics over communication interface 353. Memory 352 that is non-transitory such as volatile and/or non-volatile memory for storing instructions, information, and collected data. Instructions are stored in memory 352 in the form of software and/or firmware that is executable by processing electronics 351 in its locations throughout electronics 350. Instructions having different functions can be performed in the locations within electronics 350 most conducive to those functions. These instructions can be embedded, installed, or both embedded and installed in the electronics. Many types of memory can be used in receiver unit 20 including, but not limited to random access memory (RAM) (e.g., Dynamic RAM (DRAM), Static RAM (SRAM), Synchronous Dynamic RAM (SDRAM), Double Data Rate SDRAM (DDR SDRAM), Magnetoresistive RAM (MRAM), Resistive RAM (RRAM)), read only memory (ROM) (e.g., flash memory, Programmable ROM (PROM), Erasable PROM (EPROM), Electrically Erasable PROM (EEPROM)), variants thereof (e.g., virtual memory, register memory, cache memory), and/or others. Memory 352 can be in the form of one or more discrete memory chips, can be distributed throughout the semiconductor chips of receiver unit 20, such as within a processor chip and within a wireless communications chip, or can be both in the form of one or more discrete chips and distributed throughout one or more chips.

Receiver electronics 350 include wireless communication electronics 354 in communication with processing electronics 351 and memory 352 over communication interface 353. Wireless communication electronics 354 include hardware and software for performing communications with the various types of OBDs 100 and communication networks. Processing electronics 354, in whole or in part, can be integrated within wireless communication electronics 352 for efficient handling of communications tasks. In all of the embodiments herein, wireless communication electronics 354 are configured to communicate with on-body devices 100 according to a Bluetooth Low Energy protocol. In all of the embodiments herein, wireless communication electronics 354 can also be configured to communicate with on-body devices 100 according to a Near-Field Communication (“NFC”) protocol. In all of the embodiments herein, wireless communication electronics 354 can be configured to communicate with on-body devices 100 according to two or more communication protocols, such as BLE and NFC. However, wireless communication electronics 352 can have the hardware and software to perform wireless communications with on-body devices 100 in accordance with the communication protocols with which they are configured to operate. These communication protocols (some referenced both here and elsewhere herein) include but are not limited to: Bluetooth; BLE; NFC; RFID; ultra-wideband (UWB); mobile protocols (e.g., Fifth Generation (5G) technology, Long Term Evolution (LTE), Global System for Mobile Communications (GSM), Code Division Multiple Access (CDMA)); Z-Wave; a personal area network according to Institute of Electrical and Electronics Engineers (IEEE) 802.15 protocols (e.g., Zigbee), Adaptive Network Topology (ANT), or IPv6 over Low-Power Wireless Personal Area Networks (6LoWPAN); IEEE 802.11 protocols (e.g., 802.11a, 802.11b, 802.11g, 802.11n (aka Wi-Fi 4), 802.11ac (aka Wi-Fi 5), 802.11ah (Wi-Fi HaLow), 802.11ax (aka Wi-Fi 6)); infrared communications according to the Infrared Data Association standards (IrDA); Light Fidelity (Li-Fi); low-power wide-area network (LPWAN) protocols such as LoRa (Long Range); and wireless communication protocols that are proprietary, customized, and not subject to standardization by an international, national, nor regional standard setting body.

Wireless communication electronics 354 can include communication circuitry for each protocol with the receiver operates. Shown here are an antenna 355 communicatively coupled with a transceiver 356. Transceiver 356 has both transmit and receive hardware and capability. Transceiver 356 can further include transmit and receiver amplification circuitry. The communication circuitry has the capability to support all the requisite communication protocols for the implementation of system 10 and can include more than one antenna and transceiver if necessary. For brevity, only a single set of the communication circuitry is illustrated in this embodiment. The transceiver 356 can be implemented as a single component or as a separate transmitter and a separate receiver, collectively referred to herein as the transceiver. The one or more transmit amplifiers can be selectively connected between an output of the transmitter of the transceiver 356 and the antenna 355. The one or more receive amplifiers are configured to be selectively connected between the antenna 355 and an input of the receiver of the transceiver 356.

Receiver electronics 350 can include a power source 357. Power source 357 can be a battery, such as a lithium-ion battery. Power source 357 supplies electrical voltage and current to all components of electronics 355 by way of a power connection 358 that can include a power bus and PCB power and ground planes. Power source 357 can be accompanied by power management circuitry for managing the connection and disconnection of the power source from the electronics 350 and/or for boosting voltage (e.g., with a charge pump).

Receiver electronics 350 includes one or more clocks 359. Clocks 359 can include circuitry to produce one or more clock signals to act as timing references for electronics 350. Clocks 359 can be communicatively coupled with processor electronics 351, wireless communication electronics 354, and other components via interface 353. Clocks 359 can be centralized in the receiver unit or distributed in various locations to serve the circuitry that requires each timing reference. The clock signals can have different frequencies from each other. The clock signals can be used, among other things, to wake the receiver unit from a sleep state and to synchronize the timing of advertising or connection events and for entering a sleep state between the advertising or connection events. The clock signals can determine when the receiver unit should wake up next after processing the advertising or connection events before going to sleep. The receiver unit can then set an event to wake up in time for the next advertising or connection events.

Communication interface 353 can be electrical traces or connections that communicatively couple two or more entities together. Interface 353 is distributed throughout the receiver unit to permit communication between all components requiring interaction. Interface 353 can include a main communication bus and one or more I/P ports. Interface 353 can be within a chip, external to a chip (e.g., on a PCB), or both.

While communication with the user is readily performed with the receiver unit, in some examples the OBD can include a user interface for communication to and from the user. The user interface can be a display, such as a touch screen display, that can both output information and accept input from the user.

The user interface can be one or more optical indicators, such as light emitting diodes. The user interface can include one or more buttons, switches, or actuators that can be activated by the user to provide input to the OBD. In such cases where the OBD includes a user interface then the OBD can be used independently in the system without a receiver unit.

Receiver device 120 can further include display and input interface electronics 360 for facilitating review of analyte data received from the on-body device 100 or other device. The display can be a power-efficient display with a relatively low screen refresh rate to conserve energy use and further reduce the cost of the receiver unit. The display can be a non-interactive screen or a low-cost touch screen that acts as an input interface to receive user input. Although not illustrated in FIG. 1R-13, receiver electronics 350 can be configured to interface with separate input interface components (e.g., buttons, physical keys, toggle switches, light sensors, and/or microphones, etc.) in addition to or as an alternative to a touchscreen. Display and input interface electronics 360 can be communicatively coupled to processing electronics 351, and other components, over communication interface 353.

### Inserters and Applicators

The analyte monitoring systems 10 described herein can include a tool for insertion of the sensor (if applicable) into and application of OBD 100 to the subject's body by a user (who can be the subject or a different person). In systems 10 utilizing partially implantable sensors 161, an inserter 60, also referred to as an insertion device, can be included.

Referring to FIGS. 1S-1 and 1S-2, a cross-sectional view of an inserter 60 is shown according to an embodiment. The inserter 60 may be provided for inserting any of the partially in vivo analyte sensors 161 described herein. The inserter 60 may be provided for positioning the housing of any embodiment of OBD 100 (configured for a partially implantable sensor described herein) on the skin of the user, such as OBD 100-1 shown in FIGS. 1B-1 to 1B-3.

The inserter 60 is configured to insert the analyte sensor 161 into the body 5. In particular, the inserter 60 is configured to insert the second portion 120 of the analyte sensor 161 into the body 5. In this embodiment, the inserter 60 is configured to insert the second portion 120 of the analyte sensor 161 under the skin and in contact with bodily fluid (e.g., interstitial fluid).

The inserter 60 is also configured to position the housing 102 of OBD 100 on the skin. Thus, the inserter 60 is configured to apply OBD 100 to the body 5 by inserting the analyte sensor 161 into the skin and placing the housing 102 on the skin. When OBD 100 is placed on the skin, the adhesive patch 106 (not shown) can adhere to the skin to attach the housing 102 to the skin.

In other examples, the inserter 60 may position the analyte sensor 161 in the body 5 as an initial step. As a subsequent step, the sensor electronics 122 can be coupled to the analyte sensor 161. For example, the coupling of the sensor electronics 122 to the analyte sensor 161 may be performed manually by the user or with the aid of a mechanical device. For example, the inserter 60 may be used with OBD 100-12 described with reference to FIGS. 1M-1 and 1M-2 to insert the analyte sensor 161 before the sensor electronics 122 are subsequently attached by the user.

In other examples, the inserter 60 may assemble the analyte sensor 161 into the housing of the OBD at the same time as insertion or before insertion. For example, the inserter 60 may be used with OBD 100-9 described with reference to FIGS. 1J-1 and 1J-2 to assemble the sensor connector 940 into the housing 902 at the same time as inserting the analyte sensor 161 into the skin. As another example, the inserter 60 may be used with OBD 100-10 described with reference to FIGS. 1K-1 and 1K-2 to assemble the sensor connector 1040 into the housing 1002 before inserting the analyte sensor 161 into the skin.

In this embodiment, the inserter 60 comprises an inserter housing 62. The inserter housing 62 may have one or more parts and contains one or more components of the inserter 60. For example, OBD 100 is placed within the inserter housing 62. The inserter housing 62 may provide a surface for placement on the skin. For example, the inserter housing may have an open end (e.g. proximal end) configured to be placed on the skin, as shown in FIG. 1S-1. The inserter housing 62 may be placed on the skin to securely insert the analyte sensor 161. The inserter housing 62 may be the outer housing (e.g., the outer surface of the inserter 60).

In this embodiment, the inserter 60 comprises a device support 64. The device support 64 is configured to support OBD 100. In FIG. 1S-1, the device support 64 is retained at the distal end 61 of the inserter housing 62. The device support 64 may be configured to grip the housing 102. For example, the device support 64 may comprise notches, protrusions, or recesses for engagement with corresponding protrusions, notches, or recesses on the housing 102. For example, the housing 102 may comprise recesses and the device support 64 may comprise protrusions for engaging the recesses. This may help securely retain OBD 100 in the inserter 60.

In this embodiment, the inserter 60 comprises a sharp 66 when ready for insertion. For example, the sharp 66 may be a needle. The sharp 66 may be used to insert the analyte sensor 161 into the body 5. In particular, the inserter 60 may be configured to insert the sharp 66 into the body 5. The analyte sensor 161 may be positioned adjacent the sharp 66 so that the analyte sensor 161 is inserted when the sharp 66 is inserted. For example, the analyte sensor 161 may be positioned within the sharp 66. In some examples, the sharp 66 has an open cross section (such as a U-shape, a V-shape, or a C-shape), and the analyte sensor 161 may be received within the opening.

In this embodiment, the sharp 66 is coupled to the device support 64. In this manner, the device support 64 is configured to support the sharp 66. Thus, movement of the device support 64 also moves the sharp 66.

In other examples, the inserter 60 comprises a sharp support. The sharp support may be configured to support the sharp 66. Thus, instead of the device support 64 supporting the sharp 66, a separate sharp support may be provided. In this example, the sharp 66 may not be coupled to the device support 64 and may move independently.

In other examples, a sharp 66 is not provided. For example, the analyte sensor 161 may be inserted without a sharp 66. For example, the tip of the analyte sensor 161 may be sharp (e.g., capable of penetrating the skin). The inserter 60 may be configured to advance the analyte sensor 161 to insert the analyte sensor 161 into the skin without use of a sharp 66. This may be referred to as a sharpless inserter.

In this embodiment, the inserter 60 comprises an insertion mechanism 68. The insertion mechanism 68 is configured to insert the sharp 66 and the analyte sensor 161 into the body 5. For example, the insertion mechanism 68 may be configured to move OBD 100 towards the skin and insert the analyte sensor 161. For example, the insertion mechanism 68 may cause OBD 100 to move within the inserter housing 62. In some examples, the insertion mechanism 68 may cause the device support 64 to move, and in turn OBD 100 is moved by the device support 64.

In other examples, OBD 100 may be positioned on the skin, and the insertion mechanism 68 may move the analyte sensor 161 into the skin (e.g., by causing a sharp support to move and in turn cause the analyte sensor 161 to move). In some examples, the inserter 60 may assemble the analyte sensor 161 into the housing 102 in this manner, such as by use of a sensor connector (e.g., the sensor connector 940).

In this embodiment, the insertion mechanism 68 comprises an insertion spring. The insertion spring is compressed in an initial state in which the analyte sensor 161 is in a distal position (e.g., at the top of the inserter housing 62). The compressed state is shown in FIG. 1S-1. The insertion spring may be retained in the compressed state, such as by a retention member (not shown). To perform the insertion, the insertion spring may be released from the compressed state. For example, the retention member may release the insertion spring. In some examples, the user may press a button (not shown) to cause the retention member to release the insertion spring. In other examples, the insertion spring may be released by other mechanisms. The insertion spring may then expand to an expanded state. The expanded state is shown in FIG. 1S-2. The expansion of the insertion spring can move the analyte sensor 161 in the proximal direction from a distal position to a proximal position, in which the analyte sensor 161 is inserted into the body 5. Thus, the insertion may be automatic.

In other examples, an insertion spring is not provided. For example, the insertion mechanism 68 may comprise a movable first part of the housing (see, e.g., FIGS. 1T-1 to 1T-3), which can also be referred to as a handle. The movable handle may be used to push the analyte sensor 161 to advance the analyte sensor 161 into the body 5. The user may apply a manual force on the movable handle to insert the analyte sensor 161. In this case, the insertion may be manual. For example, the movable handle may be configured to move relative to the inserter housing 62. For example, the movable handle may be arranged around the inserter housing 62. In this manner, the inserter housing 62 may be referred to as an inner housing or a sheath. In some examples, the movable handle may be provided and used to perform an action as part of the insertion process also utilizing a separate insertion mechanism 68. For example, the movable handle may be advanced relative to the inserter housing 62 to (at least in part) cause release of the insertion mechanism 68 from the retention member. For example, moving the movable handle by a fixed distance may automatically release the insertion mechanism 68, or allow the user to activate the insertion mechanism 68 (e.g., by pushing a button after moving the movable handle to he required position).

Once OBD 100 has been placed on the skin, the housing 102 can be released by the inserter 60. Thus, the inserter 60 may be configured to release OBD 100 on the skin. For example, the device support 64 may release the housing 102. For example, the user may remove the inserter 60 from the skin, and the adhesive force of the housing 102 on the skin may cause the device support 64 to be released from the housing 102. In other examples, the device support 64 may automatically release the housing 102 as it reaches the skin.

In some examples, the inserter 60 comprises a retraction mechanism. For example, the retraction mechanism may comprise a retraction spring. The retraction mechanism may be configured to retract the sharp 66 and remove it from the body 5. The sharp 66 may be retracted into the inserter housing 62. The retraction mechanism may also retract the sharp support if provided. In some examples, the retraction mechanism may also retract the device support 64 (e.g., if the device support 64 is coupled to the sharp 66). The retraction of the device support 64 may cause the release of the housing 102 from the device support 64. In other examples, the device support 64 may not be retracted and may remain in a proximal position.

The retraction may be performed automatically, for example in response to the analyte sensor 161 being inserted and the housing 102 being placed on the skin. For example, the retraction mechanism may be activated (e.g., the retraction spring may be released from a compressed state to an expanded state), for example by release from a retention member. Release of the retention member for the retraction spring may be performed by the insertion mechanism 68. In other examples, the retraction may be activated by the user (e.g., by pressing a button or otherwise operating the inserter 60). In other examples, there may be no retraction mechanism, and the sharp 66 may be withdrawn from the body 5 by removing the inserter 60 from the skin. In this manner, the sharp 66 may not be retracted into the inserter housing 62. Accordingly, it is preferable to provide a retraction mechanism (and more preferably automatic retraction) to protect the user from the sharp 66.

In some examples, the inserter 60 may comprise a cap. For example, the cap may be attached to and/or over an end, such as the distal end 61, of the inserter 60. In particular, the cap may be attached to the inserter over the open end of the inserter housing 62 (configured to be placed on the skin). Additionally, or alternatively, the cap may be attached over an open end of the movable handle, if provided. For example, the cap may attach to the outer housing. The inserter 60 may have an interior space in which the sharp, the sensor, and/or the on-body device is housed. The interior space may be a sterile environment. The sterile environment may be maintained by a seal coupled with a portion of the inserter 60, such as the handle and/or the housing. Also, or alternatively, the interior space may include a secondary container within the sealed inserter, such as a vial, a sheath, a sleeve, or a casing that maintains a sterile environment therein for the sensor and/or sharp, as described herein with respect to FIGS. 1U-13, 1U-14, and/or 1U-15. The seal may be, at least in part, the removable cap. Removal of the cap may expose the sterile environment. Removal of the cap may also cause removal of the secondary container from the sharp and/or sensor, such as if the secondary container is affixed to the inside of the cap and is removed with the cap. The inserter 60 may be in a shipment state for shipment to a user with the interior space maintained as a sterile environment with the seal, optionally with the sharp, the sensor, and/or the on-body device contained therein. In some examples, the cap may protect the user from the sharp 66. For example, the cap may be attached by threads, and the cap may be screwed and unscrewed from the inserter 60.

FIGS. 1T-1, 1T-2, and 1T-3 are perspective views depicting another example embodiment of an inserter 60, referred to as inserter 60-1, at stages during an insertion and application procedure on the recipient's skin 10. In this embodiment the inserter 60-1 has a first part or handle 71 that slides with respect to a second part, or housing 62. Application of a manual push force to a surface 72 (e.g., a backside) of the handle 71 telescopes handle 71 either into or over the housing 62 while housing 62 is pressed against the skin 10. The range of sliding motion of handle 71 from the initial position of FIG. 1T-1 to the advanced position of FIG. 1T-2 advances the sharp 66 within the inserter 60-1 into the skin 10 to create an opening in which the sensor 161 can be inserted. Motion of handle 71 to the advanced position also concurrently advances the sensor 161 and OBD 100 into position on the recipient's body, with the sensor 161 positioned within the opening created by the sharp 66 and OBD 100 attached to the skin 10 (FIG. 1T-2). The inserter 60-1 can then be removed from the skin 10 leaving behind the sensor 161 and OBD 100 adhered to the skin 10 as shown in FIG. 1T-3. While the sharp 66 is shown here extending from within inserter 60, in other embodiments as described above the inserter 60-1 can be configured to automatically retract the sharp 66 into a position within the inserter 60-1 (such as by release of a spring) to lessen the risk of accidental stick by the sharp 66.

FIGS. 1U-1, 1U-2, and 1U-3 are cross-sectional views depicting an example embodiment of an inserter 60-1 configured for use in an insertion process similar to that described with respect to FIGS. 1T-1, 1T-2, and 1T-3. Inserter 60-1 includes handle 71-1 and housing 62-1 slidably coupled together, device support 64-1, sharp 66-1, a sharp carrier 84-1 and a retraction mechanism 81-1 having a retraction spring 82-1. FIG. 1U-1 shows inserter 60-1 in a state ready for use to insert sharp 66-1 and sensor 161 into the body (similar to the state of FIG. 1T-1). The user moves handle 71-1 forward towards the skin and doing so moves device support 64-1 with on-body device 100 coupled thereto in corresponding fashion. FIG. 1U-2 shows handle 71-1 after moving to a deployment position, where adhesive patch 106 of on-body device 100 is adhered to the skin and sharp 66-1 and sensor 161 have penetrated into the body (similar to the state of FIG. 1T-2). At this position, a release mechanism 83-1 is triggered (e.g., by a latch or other movable or deflectable member) that releases retraction spring 82-1 allowing it to expand. Expansion of retraction spring 82-1 moves a sharp carrier 84-1 in reverse away from the skin. Sharp 66-1 is coupled with sharp carrier 84-1 and this reverse motion retracts sharp 66-1 from the skin into a position inset within inserter 60-1 as shown in FIG. 1U-3, where sharp 66-1 is no longer exposed to the user, and thus maintained in a location relatively safe from accidental sticking. Inserter 60-1 can then be removed from the skin, leaving on-body device 100 and sensor 161 in the fully deployed position (similar to the state of FIG. 1T-3, but with sharp 66 retracted).

FIGS. 1T-4, 1T-5 and 1T-6 are perspective views depicting a second example embodiment of an inserter 60-2 at stages during an insertion and application procedure. In FIG. 1T-4 the inserter 60-2 is brought towards the skin 10 with OBD 100 loaded therein. This embodiment of inserter 60-2 functions similarly to the preceding embodiment but here, after inserter 60-2 is brought into contact with skin 10, the inserter 60-2 is actuated by an actuator 73 (e.g., a depressible button or slidable mechanism) which causes internal advancement of the sharp 66 into the skin (e.g., such as by release of a spring), as well as advancement of the sensor 161 into the opening created by the sharp 66 and advancement of OBD 100 to an attached position on the skin 10 (FIG. 1T-5). Thus, the continuous manual push force applied to a surface that drives the sharp into the skin, as described with respect to FIGS. 1T-1 and 1T-2, is not required for operation of this embodiment, as the advancement of the internal components is performed automatically upon triggering of the actuator 73. The inserter 60-2 can then be removed from the skin as shown in FIG. 1T-6. While the sharp 66 is shown here as extending from within inserter 60-2, in other embodiments the inserter 60-2 can be configured to automatically retract the sharp 66 into a position within the inserter 60-2 (such as by release of a spring) to lessen the risk of accidental stick by the sharp 66.

FIGS. 1U-4, 1U-5, and 1U-6 are cross-sectional views depicting an example embodiment of an inserter 60-2 configured for use in an insertion process similar to that described with respect to FIGS. 1T-4, 1T-5, and 1T-6. Inserter 60-2 includes housing 62-2, actuator 73-2, device support 64-2, sharp 66-2, a sharp carrier 84-2, insertion mechanism 68-2 having insertion spring 85-2, and a retraction mechanism 81-2 having a retraction spring 82-2. FIG. 1U-4 shows inserter 60-2 in a state ready for use to insert sharp 66-2 and sensor 161 into the body (similar to the state of FIG. 1T-4). The user actuates actuator 73-2 which triggers a release mechanism 86-2 (e.g., a latch or other movable or deflectable member). In doing so, device support 64-2 with on-body device 100 coupled thereto is moved within inserter 60-2 forward towards the skin by action of expansion of insertion spring 85-2. FIG. 1U-5 shows device support 64-2 after moving to a deployment position where adhesive patch 106 of on-body device 100 is adhered to the skin and sharp 66-2 and sensor 161 have penetrated into the body (similar to the state of FIG. 1T-5). At this position, a release mechanism 83-2 is triggered (e.g., by a latch or other deflectable member) that releases retraction spring 82-2 allowing it to expand. Expansion of retraction spring 82-2 moves a sharp carrier 84-2 in reverse away from the skin. Sharp 66-2 is coupled with sharp carrier 84-2 and this reverse motion retracts sharp 66-2 from the skin into a position inset within inserter 60-2 as shown in FIG. 1U-6, where sharp 66-2 is no longer exposed to the user, and thus maintained in a location relatively safe from accidental sticking. Inserter 60-2 can then be removed from the skin, leaving on-body device 100 and sensor 161 in the fully deployed position (similar to the state of FIG. 1T-6, but with sharp 66 retracted).

With respect to inserter 60 for use with systems having a partially implanted sensor 161, the inserter 60, sensor 161, and OBD 100 can be provided to the end-user in various states of assembly. Each component 60, 161, and 100 is subjected to a sterilization process and placed in a shipment state (e.g., sealed packaging or container) for the transfer from the manufacturer's possession to another party downstream, such as a distributor, doctor's office, or end user. The sterilization process for sensor 161, OBD 100, and inserter 60 can be a Spaulding critical level sterilization process, such as by ionizing (gamma) radiation or poison (e.g., ethylene oxide (EtO)). The sterilization processes can be performed independently on each component, or they can be sterilized together. Each component remains sterile while in their shipment states.

The inserter 60, sensor 161, and OBD 100 can be placed in their shipment state while not connected or combined. The end user can assemble the components prior to using the inserter 60 to perform the insertion and application procedure. The user can couple the sensor 161 with OBD 100 into a connected assembly, and then load the connected assembly into the inserter 60, and then perform insertion and application procedure.

OBD 100 and sensor 161 can be coupled together by the manufacturer into a connected assembly. The connected assembly can be placed in its shipment state while not loaded within inserter 60. The end user, after receipt, can load the connected assembly into the inserter 60, and then perform insertion and application procedure.

OBD 100 can be loaded into the inserter 60 by the manufacturer to form a pre-loaded assembly and the pre-loaded assembly can be placed in its shipment state. The inserter 60 can include a seal that seals the OBD 100 within the inserter 60. The removable cap, if provided, can be attached to the inserter 60 and can act as the seal. The sensor 161 can be placed in a sealed container (e.g., a carrier or tray) in its shipment state separate from the pre-loaded assembly (OBD 100 and inserter 60). The end user, after receipt, can load the sensor 161 into OBD 100 while within the inserter 60, and then perform the insertion and application procedure.

FIGS. 1U-7 and 1U-8 are cross-sectional views depicting an example embodiment of mating an inserter 60 holding an on-body device 100 therein with a sensor 161 contained within a discrete container 88. In this embodiment, inserter 60 includes housing 62, device support 64, and optionally an insertion mechanism 68 and/or retraction mechanism 81. Sensor 161 is held within a sharp 66 by a sensor connector 87 (e.g., similar to sensor connector 1040) that is held in a seat 89 within container 88. A lid 90 of container 88 is removed, and then inserter 60 can be aligned with container 88 and advanced as depicted in FIG. 1U-7. Device support 64, or another element of inserter 60 such as a sharp carrier, can grasp a sharp hub 91 of sensor connector 87 within container 88 and on-body device 100 and sensor connector 87 can secure themselves together. Removal of inserter 60 from container 88 removes sensor connector 87 from seat 89. Inserter 60 is then in a state ready to deploy the on-body device 100 (with sensor connector 87 having sensor 161 coupled thereto). After deployment, sensor connector 87 remains with on-body device 100 and sharp 66 can be retracted by inserter (if desired) by way of removal of hub 91. In some embodiments container 88 can be a tray that is provided to the user in the position of an end cap of the inserter such that the two assemblies are attached, and there is no need for a separate lid 90. Container 88 may include a magnet 69 (e.g., a permanent magnet) in a location that exerts a magnetic field sufficient to be detected by an activation switch 321, configured as a magnetic switch, of the on-body device 100. When the on-body device 100 is moved in proximity with magnet 69 during the sensor connector mating process, the magnetic field can be detected by the activation switch 321 which in turn activates at least a portion of the on-body device's sensor electronics 122 to begin an activation process to ready for implantation.

The sensor 161 can be loaded into the inserter 60 by the manufacturer to form a pre-loaded assembly and the pre-loaded assembly can be placed in its shipment state. The inserter 60 can include a seal that seals the sensor 161 within the inserter 60. The removable cap, if provided, can be attached to the inserter 60 and can act as the seal. OBD 100 can be placed in a sealed container (e.g., a carrier or tray) in its shipment state separate from the pre-loaded assembly (sensor 161 and inserter 60). The end user, after receipt, can load OBD 100 into the inserter 60. OBD 100 can be loaded into the inserter 60 such that the sensor 161 and OBD 100 are placed in their connected state, or the two can be connected by the inserter 60 during the insertion process. The user can then perform the insertion and application procedure.

FIGS. 1U-9 and 1U-10 are cross-sectional views depicting an example embodiment of mating an inserter 60 holding sensor connector 87 therein with an on-body device 100 contained within a discrete container 88. This embodiment is similar to the embodiment of FIGS. 1U-7 and 1U-8 but the positions of the sensor connector 87 and on-body device 100 are reversed. Here, inserter 60 again includes housing 62, device support 64, and optionally an insertion mechanism 68 and/or retraction mechanism 81. Sensor 161 is held within sharp 66 by sensor connector 87 (e.g., similar to sensor connector 1040) that is held by inserter 60, such as by device support 64 and/or a sharp carrier (not shown). On-body device 100 having adhesive patch 106 is held in a seat 89 within container 88. A lid 90 of container 88 is removed, and then inserter 60 can be aligned with container 88 and advanced as depicted in FIG. 1U-9. Device support 64 can grasp on-body device 100 within container 88 such that connector 87 and on-body device 100 are secured together. Removal of inserter 60 from container 88 removes on-body device 100 from seat 89. Inserter 60 is then in a state ready to deploy the on-body device 100 (with sensor connector 87 having sensor 161 coupled thereto). After deployment, sensor connector 87 remains with on-body device 100 and sharp 66 can be retracted by inserter (if desired) by way of removal of hub 91. In some embodiments container 88 can be a tray that is provided to the user in the position of an end cap of the inserter such that the two assemblies are attached, and there is no need for a separate lid 90. Container 88 may include a magnet 69 (e.g., a permanent magnet) in a location that exerts a magnetic field sufficient to be detected by an activation switch 321, configured as a magnetic switch, of the on-body device 100. When the on-body device 100 is removed from proximity with magnet 69 after completion of the sensor connector mating process, the absence of the magnetic field can be detected by the activation switch 321 which in turn activates at least a portion of the on-body device's sensor electronics 122 to begin an activation process to ready for implantation.

FIGS. 1U-11 and 1U-12 are cross-sectional views showing a related embodiment. Here, the on-body device 100 can again be provided in container 88 and insertion of inserter 60 into container 88 can again cause device support 64 to grasp on-body device 100. FIG. 1U-11 shows the inserter 60 and container 88 prior to removal of lid 90 and coupling of support 64 with device 100. FIG. 1U-12 shows inserter 60 after coupling, where on-body device is now held by device support 64 at the distal end of inserter housing 62. Insertion mechanism 68 and/or retraction mechanism 81 is coupled with sensor connector 87 having sharp 66 and sensor 161 and holds connector 87 in spaced relation from on-body device 100 such that they have not yet been assembled together. Upon actuation of inserter 60 (with manual or automatic advancement), sensor connector 87 is advanced to concurrently place and secure sensor connector 87 within recess 92 of on-body device 100 while penetrating the body with sharp 66 and placing sensor 161 into the partially implanted position with on-body device 100 adhered to the skin. In this embodiment, on-body device 100 need not move within inserter 60 and can remain in a position with adhesive patch 106 exposed for contacting the skin at the distal end of housing 62.

In the embodiments described with respect to FIGS. 1U-9, 1U-10, 1U-11, and 1U-12, on-body device 100 can be provided in container 88 with a liner attached to the skin-contacting side of adhesive patch 106. Container 88 may be configured such that removal of on-body device 100 from container 88 also causes removal of the liner from the adhesive patch 106 (such as by securing the liner to seat 89 of container 88). Such a configuration can prolong the life of the adhesive by minimizing the amount of time exposed to external environment, including the environment within container 88 itself, where the adhesive can dry out and/or collect debris.

The OBD 100 with sensor 161 can be loaded into the inserter 60 by the manufacturer to form a pre-loaded assembly and the pre-loaded assembly can be placed in its shipment state. The inserter 60 can include a seal that seals the OBD 100 with sensor 161 within the inserter 60. The removable cap, if provided, can be attached to the inserter 60 and can act as the seal. The end user, after receipt, does not need to load any component into the inserter 60 as the shipment state is a ready-to-use state. The user can remove the seal, e.g., the cap, and then perform the insertion and application procedure.

FIG. 1U-13 is a perspective view of an example embodiment of an inserter 60 (which may also be an applicator 77 or 79 as described herein with respect to FIGS. 1T-10 through 1T-13) having a removable end cap 93. FIGS. 1U-14 and 1U-15 are cross-sectional views depicting this embodiment of inserter 60 configured for insertion of partially implantable sensor 161 in an embodiment where the inserter 60 is provided to the end user with on-body device 100, sensor 161, and sharp 66 arranged therein as a sterile pre-loaded assembly. Inserter 60 can otherwise be configured in accordance with any of the embodiments described herein, such as with an insertion mechanism 68 and/or a retraction mechanism 81. In the embodiment of FIGS. 1U-14 and 1U-15, end cap 93 includes a sheath 94 configured to receive sharp 66 and sensor 161. An end 95 of sheath 94 is coupled to on-body device 100 in a manner such that a seal is formed, where that seal can be broken or otherwise opened upon removal of end cap 93 from inserter 60. End 95 can be bonded to on-body device, coupled to on-body device mechanically (such as with threads), and/or can be integrally formed with the material of a component of on-body device 100 such as in the form of a thin breakable plastic seal. Sheath 94 has an inner space 96 that encompasses sharp 66 and sensor 161 and, when enclosed by the sealed end 95, can maintain a sterile environment for sharp 66 and sensor 161 as shown in FIG. 1U-14. The sterile environment can be created as desired, such as by irradiation (e.g., electron beam or “e-beam” irradiation, or low-energy X-ray irradiation). When cap 93 is removed, the sealed end 94 is broken, uncoupled, and/or otherwise separated from on-body device 100, and sheath 94 is removed along with end cap 93 as depicted in FIG. 1U-15. The inserter 60 can then be used for the insertion process.

End cap 93 may include a magnet 69 (e.g., a permanent magnet) in a location that exerts a magnetic field sufficient to be detected by an activation switch 321, configured as a magnetic switch, of the on-body device 100. When the end cap 93 is removed from housing 62 or other portion of inserter 60, the absence of the magnetic field can be detected by the activation switch 321 which in turn activates at least a portion of the on-body device's sensor electronics 122 to begin an activation process to ready for implantation.

Thus, for the partially implantable sensor embodiments described herein, the components 60, 161, and 100 can be assume one of a variety of pre-loaded configurations when placed in the shipment state, which is then ultimately received by the end user (e.g., the recipient or other person).

An inserter or applicator can include one or more projections for engaging one or more recesses of a housing of on-body device 100, and/or can include one or more recesses for engaging one or more projections of an on-body device 100. Any number of one or more (e.g., two, three, four, or more) recesses and/or projections can be present on-body device 100 and/or the inserter or applicator. FIG. 1U-16 is a perspective view depicting an example embodiment of a device support 64 configured to engage with an on-body device. In this embodiment, a sidewall of device support 64 includes deflectable arms 97 each with a projection 98-1, such as a detent, configured to be received by recesses 99-1 in corresponding positions on housing 102 of on-body device 100. FIG. 1U-17 is a close-up cross-sectional view showing projection 98 received within a recess of on-body device 100.

Inserter 60 may include a magnet 69 (e.g., a permanent magnet) in a location that exerts a magnetic field sufficient to be detected by an activation switch 321, configured as a magnetic switch, of the on-body device 100. In this embodiment, the magnet 69 is coupled with device support 64. When the on-body device 100 is released from device support 64 after positioning on the wearer's body, the removal and absence of the magnetic field can be detected by the activation switch 321 which in turn activates at least a portion of the on-body device's sensor electronics 122 to begin an activation process to ready for implantation. In an another embodiment, the magnet 69 can be in a fixed location within inserter 60 that is not in device support 64, such that the magnet 69 is in proximity with activation switch 321, configured as a magnetic switch, and exerting the magnetic field on switch 321 when on-body device 100 is in a pre-insertion position within inserter 60, and then upon actuating inserter 60 when device support 64 carries on-body device 100 away from magnet 69 the activation switch 321 is switched and the on-body device 100 is activated.

FIG. 1U-19 is a perspective view depicting another example embodiment of a device support 64 configured to engage with an on-body device. In this embodiment, a interior surface of a sidewall of device support 64 includes a projection 98-2 in the form of a rim or ridge, which can extend along a portion or the entirety (360 degrees) of the inner surface of the sidewall, and is configured to be received by a recess 99-2 in a corresponding position on on-body device 100. FIG. 1U-20 is a close-up cross-sectional view showing projection 98-2 received within recess 99-2 of on-body device 100.

FIG. 1U-20 is a top down view of an on-body device 100 configured to engage a component of inserter 60 by way of a latch mechanism 231 around or in proximity with sharp aperture 124. Latch mechanism 231 includes a keyed interface 232 on a lip 233. An inserter component (shown here as device support 64, but alternatively can be another component) can have a cylindrical shape with projections 234 complementary to the keyed interface 232. FIG. 1U-21 is a perspective view of latch mechanism 231 and device support 64 in closer detail. Device support 64 can be aligned and advanced over lip 233 and rotated to lock projections 234 under lip 233 of latch mechanism 231 as shown in the cross-sectional view of FIG. 1U-22. The inserter can be configured to rotate device support 64 in alternate fashion to the aligned state to accomplish release of the on-body device 100 (e.g., on the skin). The latch mechanism 231 can be used in addition to, or alternatively from, engagement elements like those described with respect to FIGS. 1U-16 through 1U-19.

Sharp 66 can be configured with a sharp distal tip portion that penetrates the skin with relatively minor tissue damage, e.g., limited tearing, while maintaining adequate column strength and space for accompaniment of sensor 161. FIG. 1U-23 is a side view showing an example embodiment of a sharp 66-1. Sharp 66-1 is generally tubular, but can have a slot or opening along some or all of its shaft length to permit loading and/or unloading of sensor 161. Sharp 66-1 can be used with any of the embodiments of sensor 161 (e.g., 161A and/or 161B) disclosed herein. Sharp 66-1 has a sharp distal terminus 660 formed by a bevel. The bevel can have an angle 661 of between, for example, 15 and 55 degrees and, in another embodiment, between 20 and 45 degrees.

End on views of various examples of different embodiments of sharps 66 are shown in FIGS. 1U-24, 1U-25, 1U-26, 1U-27, and 1U-28. Sharp 66 can have an enclosed distal portion as shown in the embodiments of FIG. 1U-24 and FIG. 1U-25. Sharp 66 can have a generally circular profile to the sharp body 662 as shown in FIG. 1U-24 or a polygonal profile to sharp body 662, such as with rounded corners, as shown in FIG. 1U-25. Sharp 66 can have an open distal portion as shown in the embodiments of FIGS. 1U-26, 1U-27, and 1U-28. Sharp 66 can have a C-shape that is generally curved along its width as shown in FIG. 1U-26. Sharp 66 can have a C-shape or U-shape with sidewalls 663 and 665 extending from a floor 664. The sidewalls 663 and 665 and floor 664 can be generally curved as shown in FIG. 1U-26, can have generally straight sidewalls 663 and 665 extending from generally straight floor 664 at acute angles as shown in FIG. 1U-27, can have generally straight sidewalls 663 and 665 extending from the floor 664 at perpendicular angles as shown in FIG. 1U-28, and/or can have another combination of curved and/or straight sidewalls and floor at various.

FIG. 1U-29 is a top down view and FIG. 1U-30 is a side view of sharp 66-2 configured with the profiles of FIG. 1U-27 or 1U-28. Here, sharp 66-2 has a distal tip portion 667 with a distal terminus 660. Distal tip portion 667 is at least partially bevelled to have a V-shape with an angle 661 of between 20 and 60 degrees between edges of the bevel. In another embodiment, angle 661 is between 20 and 30 degrees. In another embodiment, angle 661 is between 27 and 37 degrees. In another embodiment, angle 661 is between 28 and 38 degrees. In another embodiment, angle 661 is between 30 and 40 degrees. In another embodiment, angle 661 is between 31 and 41 degrees. In another embodiment, angle 661 is between 45 and 55 degrees. A transition portion 668 is present where sidewalls 663 and 665 slope upwards from floor 664 at an angle 669 of between 7 and 35 degrees. In another embodiment, angle 669 is between 7 and 17 degrees. In another embodiment, angle 669 is between 8 and 18 degrees. In another embodiment, angle 669 is between 13 and 23 degrees. In another embodiment, angle 669 is between 18 and 28 degrees. In another embodiment, angle 669 is between 20 and 30 degrees. Sidewalls 663 and 664 at their full height can extend towards a proximal part of the sharp shaft as far as desired in the implementation. Sharp 66 can optionally include a second bevelled portion in floor 664 between portions 667 and 668.

FIGS. 1T-7, 1T-8, and 1T-9 are perspective views depicting an example embodiment of an inserter 74 for insertion of a wholly implantable sensor 162 through the skin 10 and into the subject's body. In FIG. 1T-7, a medical professional has created an incision 15 in the skin 10 and then a pocket 16 within the recipient's body under the skin 10. Inserter 74 has an elongate portion 75 having sensor 162 detachably mounted thereon. Elongate portion 75 and sensor 162 are inserted through incision 15 and sensor 162 is positioned within pocket 16 as shown in FIG. 1T-8. Sensor 162 is then released from portion 75 by actuation of an actuator 76 (e.g., a depressible button or slidable release mechanism) on inserter 74 and portion 75 is withdrawn from within body 5 as shown in FIG. 1T-9. Incision 15 is then closed by suture or other means.

OBD 100 can then be placed on the skin 10 over sensor 162. Wireless communication electronics in OBD 100 and sensor 162 can be used to sense when OBD 100 is optimally located over sensor 162, and this positioning feedback can be output to the user by OBD 100 (e.g., visually, audibly, and/or in tactile manner) or can be relayed to a receiver unit (not shown) for outputting to the user therefrom (e.g., visually, audibly, and/or in tactile manner). Once the optimal position is determined, OBD 100 can be attached to the skin 10. This can be performed by the user manually holding OBD 100 and directly pressing it against the skin 10 to adhere OBD 100 to the skin 10.

In an alternative embodiment, an applicator 77 can have OBD 100 held therein and can be used to apply OBD 100 to the skin 10 in the appropriate location. FIGS. 1T-10 and 1T-11 are perspective views depicting an example embodiment of an applicator 77 during and after application of OBD 100 over the implant location of sensor 162, respectively. This applicator 77 can be configured like that of inserter 60 with either the manual advancement configuration of FIGS. 1T-1, 1T-2, and 1T-3 or the automatic release configuration of FIGS. 1T-4, 1T-5, and 1T-6, but in both embodiments without the sharp 66 and sensor 161 as such are not needed. Here applicator 77 has an actuator 78 for automatic deployment of OBD 100 to the skin 10.

With respect to inserter 74 for use with systems having a wholly implantable sensor 162, the inserter 74, sensor 162, OBD 100, and applicator 77 (if utilized) can be provided to the end-user in various states of assembly. Each component 74, 162, 100, and 77 is subjected to a sterilization process and placed in a shipment state (e.g., sealed packaging or container) for the transfer from the manufacturer's possession to another party downstream, such as a distributor, doctor's office, or end user. The sterilization process for sensor 162, OBD 100, inserter 74, and applicator 77 can be a Spaulding critical level sterilization process, such as by ionizing (gamma) radiation or poison (e.g., ethylene oxide (EtO)). The sterilization processes can be performed independently on each component, or they can be sterilized together. Each component remains sterile while in their shipment states.

The inserter 74, sensor 162, OBD 100, and applicator 77 (if utilized) can be placed in their shipment state while not connected or combined. The user can couple the sensor 162 with the inserter 74 into a connected assembly and then perform insertion procedure. The user can load OBD 100 into the applicator 77 (if used) and then perform the application procedure.

Alternatively, the sensor 162 can be coupled with the inserter 74 by the manufacturer to form a pre-connected assembly and the pre-connected assembly can be placed in its shipment state for transfer from the manufacturer's possession. The end user, after receipt, can then perform the insertion procedure without need to connect the sensor 162 to the inserter 74 beforehand. OBD 100 and applicator 77 can be placed in their shipment states independently while unloaded, and the end user can load OBD 100 into the applicator 77 and then perform the application procedure. In an alternative embodiment, OBD 100 can be loaded into applicator 77 by the manufacturer and then placed in the shipment state as a pre-loaded assembly. The end user can perform the application procedure without the need to first load OBD 100 into the applicator 77.

An OBD 100 having a wholly ex vivo sensor can be applied manually by direct placement by a user's hand or by use of an applicator. FIGS. 1T-12 and 1T-13 are perspective views depicting the application of an OBD 100 having a wholly ex vivo sensor 163 with an applicator 79 during and after application, respectively. Like applicator 77, applicator 79 can have OBD 100 held therein and can be used to apply OBD 100 to the skin 10 in a desired location, though wireless positioning assistance is not needed. Applicator 79 can be configured like that of inserter 60 with either the manual advancement configuration of FIGS. 1T-1, 1T-2, and 1T-3 or the automatic release configuration of FIGS. 1T-4, 1T-5, and 1T-6, but in both embodiments without the sharp 66 and sensor 161 as such are not needed. Here applicator 79 has an actuator 80 for automatic deployment of OBD 100 to the skin 10.

The sensor 163, OBD 100, and applicator 79 (if utilized) can be provided to the end-user in various states of assembly. Each component 163, 100, and 79 is subjected to a sterilization process and placed in a shipment state (e.g., sealed packaging or container) for the transfer from the manufacturer's possession to another party downstream, such as a distributor, doctor's office, or end user. The sterilization process for sensor 163, OBD 100, and applicator 79 can be a Spaulding critical level sterilization process (though not required), such as by ionizing (gamma) radiation or poison (e.g., ethylene oxide (EtO)). The sterilization processes can be performed independently on each component, or they can be sterilized together. Each component remains sterile while in their shipment states.

The sensor 163, OBD 100, and applicator 79 (if utilized) can be placed in their shipment state while not connected or combined. The user can couple the sensor 163 with OBD 100 into a connected assembly and then load the connected assembly into the applicator 79 and perform the application procedure.

The sensor 163 and OBD 100 can be connected by the manufacturer and placed in their shipment state while connected. The applicator 79 can be separately placed in its shipment state. After receipt, the user can load the connected assembly of sensor 163 and OBD 100 into the applicator 79, and then perform the application procedure.

The sensor 163 and OBD 100 can be connected by the manufacturer and placed in their shipment state while connected. The manufacturer can load the connected assembly of sensor 163 and OBD 100 into the applicator 79, and then place the entire connected and loaded assembly into its shipment state. After receipt, the user can perform the application procedure without the need to connect sensor 163 and OBD 100, and without the need to first load the applicator 79.

### Vent Passageway

The following section describes various embodiments of an OBD 100 having a vent passageway 111. The term “vent passageway 111” refers collectively to all embodiments of a vent passageway, including vent passageways 111-1 through 111-10. This section includes a description of OBDs 100-50 to 100-72 referring to FIGS. 2A to 2K-12. Any two or more of the vent passageways 111 described with respect to OBDs 100-50 to 100-72 in FIGS. 2A to 2K-12 can be combined in a single embodiment of OBD 100, as repeated below.

### On-Body Device 100-50

Referring to FIGS. 2A-1 to 2A-4, an OBD 100-50 is shown according to an embodiment. OBD 100-50 is similar to OBD 100-1 of FIGS. 1B-1 to 1B-3, and may include the same features, except as explained below. The optional features and alternatives described in relation to OBD 100-1 may similarly be applied to OBD 100-50 in a corresponding manner. Similarly, the features described in relation to OBDs 100-1′ to 100-32 may be applied to OBD 100-50. OBD 100-50 may be used as part of the analyte monitoring system 10. For example, OBD 100-50 may be used with at least one receiver unit 20.

In this embodiment, OBD 100-50 is the same as OBD 100-1, except that OBD 100-50 has a housing 10002-1 which includes a vent passageway 111 and associated features. The term “housing 10002” refers collectively to all embodiments of housings described herein, including housings 10002-1 through 10002-10 described hereafter. In this embodiment, the housing 10002-1 comprises a base surface 10004, which is similar to the base surface 104, except as described below. The base surface 10004 is configured for placement on the body 5 of the user, in particular on the skin. The base surface 10004 comprises sensor aperture 112. In this embodiment, the central axis A passes through the sensor aperture 112. In other examples, the sensor aperture 112 may not be aligned with the central axis A. Instead, the sensor aperture 112 may be arranged off-center, such as towards one side of the base surface 10004.

OBD 100-50 comprises adhesive patch 106. The adhesive patch 106 is coupled to the base surface 10004. The adhesive patch 106 is configured to attach the housing 10002-1 to the skin of the user. The adhesive patch 106 comprises adhesive aperture 114. The adhesive aperture 114 is aligned with the sensor aperture 112 (in this case along the central axis A). The adhesive aperture 114 may be referred to as a first adhesive aperture.

In this embodiment, the housing 10002-1 comprises a top surface 10008, which is similar to the top surface 108, except as described below. The top surface 10008 comprises sharp aperture 124. In this embodiment, the central axis A passes through the sharp aperture 124. In other examples, the sharp aperture 124 may not be aligned with the central axis A. Instead, the sharp aperture 124 may be arranged off-center, such as towards one side of the top surface 10008.

In this embodiment, the sharp aperture 124 is aligned with the sensor aperture 112 (in this case along the central axis A). In other examples, both the sharp aperture 124 and the sensor aperture 112 are not aligned with the central axis A (e.g., off-center) and may be aligned with each other in a direction parallel to the central axis A. In other examples, the sharp aperture 124 may be offset from the sensor aperture 112 (e.g., not aligned with each other in a direction parallel to the central axis A). The sharp aperture 124 is connected to the sensor aperture 112 by a sensor passageway 126.

In this embodiment, the housing 10002-1 comprises a side surface 10010, which is similar to the side surface 110. The side surface 10010 connects the base surface 10004 to the top surface 10008. In this embodiment, the side surface 10010 extends in a direction parallel to the central axis A. In other examples, other shapes are possible, as described herein. For example, the side surface 10010 may be angled (e.g., see FIG. 1D) or may be curved (e.g., see FIG. 1E). In some examples, the side surface 10010 may be omitted and the top surface 10008 may be connected to the base surface 10004 (e.g., see FIG. 1F). In other examples, the housing 10002-1 may comprise an intermediate surface connected between the base surface 10004 and the side surface 10010 or between the side surface 10010 and the top surface 10008, which may be angled or curved, for example (e.g., see FIGS. 1G and 1H).

In this embodiment, the housing 10002-1 is generally circular. In particular, the base surface 10004 is circular. The top surface 10008 is also circular. The side surface 10010 extends circumferentially around the housing 10002-1 between the base surface 10004 and the top surface 10008. This forms a disc shape as shown in FIG. 2A-2. Further details of the shape of the housing 10002-1 are described with reference to the housing 102. In other examples, the shape of the housing 10002-1 may be different, such as described with reference to the housing 102. For example, the housing 10002-1 may be square (e.g., see FIGS. 1C-1 and 1C-2) or another shape. In this embodiment, the adhesive patch 106 is also circular and protrudes beyond the base surface 10004. In other examples, the adhesive patch 106 may have a different shape, such as similar to or different from the size and peripheral outline of the base surface 10004.

OBD 100-50 may be assembled in a variety of ways. For example, OBD 100-50 may assembled using a first part and a second part (e.g., see FIG. 1I). In another example, OBD 100-50 may be assembled with a sensor connector inserted into the top surface (e.g., see FIGS. 1J-1 to 1J-2). In another example, OBD 100-50 may be assembled with a sensor connector inserted into the base surface (e.g., see FIGS. 1K-1 to 1K-2). In another example, OBD 100-50 may be assembled with a sensor inserted into the base surface and retained using a filling material (e.g., see FIGS. 1L-1 to 1L-2). In another example, OBD 100-50 may be assembled with an electronics unit attached after insertion of the analyte sensor (e.g., see FIGS. 1M-1 to 1M-2).

OBD 100-50 comprises analyte sensor 161. The analyte sensor 161 is configured to be inserted into the body 5 of the user (e.g., into interstitial fluid of the user). In other examples, the analyte sensor 161 may be in contact with dermal fluid or blood. Further details of the analyte sensor 161 are provided with reference to FIGS. 1O-1 through 1Q-2. In this embodiment, the analyte sensor 161 is configured to detect glucose. In other examples, the analyte sensor 161 but may be used to detect other analytes as described herein. OBD 100-50 is shown and described with a partially implantable sensor but can be configured to operate with a wholly implantable sensor or wholly ex vivo sensor, in which case the adhesive aperture, sensor aperture, sensor passageway, and sharp aperture can be omitted.

OBD 100-50 comprises sensor electronics 122 further details of which are provided with reference to FIG. 1R-1.

The analyte sensor 161 is connected to the sensor electronics 122 to provide the signal generated by the analyte sensor 161 to the sensor electronics 122. The analyte sensor 161 extends into the sensor passageway 126 and protrudes through the sensor aperture 112 (and the adhesive aperture 114) from the housing 10002-1 for insertion into the body 5. The sharp aperture 124 is configured to receive a sharp (e.g., from an inserter, such as the inserter 60) for inserting the analyte sensor 161 into the body 5. In other examples, a sharp aperture 124 may not be provided.

In this embodiment, the housing 10002-1 comprises a proximal side 128 and a distal side 130. In this embodiment, the base surface 10004 is arranged at the proximal side 128. The top surface 10008 is arranged away from the proximal side 128. In this embodiment, the top surface 10008 is arranged at the distal side 130. The base surface 10004 is arranged away from the distal side 130.

Unlike the base surface 104, the base surface 10004 further comprises a vent inlet 121-1. Reference to “vent inlet 121” elsewhere collectively refers to all embodiments of vent inlets described herein, including vent inlets 121-1 through 121-10. The vent inlet 121 is an aperture in the base surface 10004. The vent inlet 121 may be referred to as a third aperture or a vent inlet aperture. The vent inlet 121-1 is an opening in the base surface 10004. In this embodiment, the vent inlet 121-1 is a circular hole. In other examples, other shapes may be provided such as a square hole or keyhole. The vent inlet 121-1 is an opening that permits passage through the base surface 10004.

The vent inlet 121-1 is configured to allow water to pass therethrough. In particular, the vent inlet 121-1 is configured to allow water vapor to pass. Water vapor is the gaseous form of water, in contrast to the liquid form. In some examples, the vent inlet 121-1 may be configured to allow liquid water to pass (for example, in addition to water vapor). For example, any fluid (e.g., gas or liquid) found in or on the body 5 of the user (e.g., water, sweat, blood) can be vented away from the user's body 5. This can mitigate degradation of the adhesive patch 106, maintaining the integrity of the adhesive attachment of OBD 100-50 on the skin. This may also inhibit a moist environment in which pathogens might propagate.

In this manner, when moisture (e.g., water) accumulates at the base surface 10004, such as from the user sweating, or due to water from showering, the water can be removed from the base surface 10004 by passing through the vent inlet 121-1. In some examples, liquid water can evaporate and pass through the vent inlet 121-1 as water vapor.

The adhesive patch 106 further comprises an adhesive vent aperture 10074. The adhesive vent aperture 10074 may be referred to as a second adhesive aperture. The adhesive vent aperture 10074 is an opening in the adhesive patch 106. In this embodiment, the adhesive vent aperture 10074 is a circular hole. In other examples, other shapes may be provided such as a square hole or keyhole. The adhesive vent aperture 10074 is aligned with the vent inlet 121-1 in the base surface 10004 along a direction parallel to the central axis A. In this example, the adhesive vent aperture 10074 is the same size and shape as the vent inlet 121-1. In other examples, the adhesive vent aperture 10074 may have a different size (e.g., smaller or larger than the vent inlet 121-1) and/or may have a different shape.

The adhesive vent aperture 10074 extends through the thickness of the adhesive patch 106. This forms an opening between the housing 10002-1 and the skin, when OBD 100-50 is placed on the skin. In this embodiment, the vent inlet 121-1 is in fluid communication with the skin (e.g., communication such that gas or liquid may flow in fluidic manner therebetween). This allows water from the skin to pass directly into the vent inlet 121-1 through the adhesive vent aperture 10074.

Providing a vent inlet 121-1 is beneficial since it can reduce accumulation of moisture between the housing 10002-1 and the skin. As a result, the vent inlet 121-1 can allow moisture to be removed from the adhesive patch 106. The vent inlet 121-1 can also reduce moisture collecting on the base surface 10004 which could otherwise pass into the adhesive patch 106.

Excess moisture can be detrimental to the effectiveness of the adhesive patch 106. Accordingly, the vent inlet 121-1 can allow for improved attachment of OBD 100-50 to the skin. In turn, this can allow for extended wear of OBD 100-50.

Unlike the top surface 108, the top surface 10008 further comprises a vent outlet 131-1. Reference to “vent outlet 131” elsewhere collectively refers to all embodiments of vent outlets described herein, including vent outlets 131-1 through 131-10. The vent outlet 131-1 is an aperture in the top surface 10008. The vent outlet 131-1 is an opening in the top surface 10008. In this embodiment, the vent outlet 131-1 is a circular hole. In other examples, other shapes may be provided such as a square hole or keyhole. The vent outlet 131-1 is an opening that permits passage through the top surface 10008.

The vent inlet 121-1 is fluidly connected to the vent outlet 131-1. In particular, the vent inlet 121-1 is connected to the vent outlet 131-1 through the housing 10002-1. This allows water (e.g., water vapor) to pass from the vent inlet 121-1 to the vent outlet 131-1. The vent outlet 131-1 is configured to allow water to pass therethrough. In particular, the vent outlet 131-1 is configured to allow water vapor to pass. In some examples, the vent outlet 131-1 may be configured to allow liquid water to pass (for example, in addition to water vapor). Water from the base surface 10004 and/or the adhesive patch 106 can pass through the vent inlet 121-1 and then through the vent outlet 131-1 for release from the housing 10002-1 through the top surface 10008.

In this embodiment, the housing 10002-1 comprises a vent passageway 111-1. The vent passageway 111-1 extends between the vent inlet 121-1 and the vent outlet 131-1. In other words, the vent inlet 121-1 is connected to the vent outlet 131-1 by the vent passageway 111-1. The vent inlet 121-1 may be referred to as a vent passageway inlet, a vent passageway first aperture, or a vent passageway first opening. The vent outlet 131-1 may be referred to as a fourth aperture, vent outlet aperture, vent passageway outlet, a vent passageway second aperture, or a vent passageway second opening. The vent passageway 111-1 may be referred to as a vent passage, a vent conduit, a vent duct, a vent lumen, or a vent. The vent passageway 111-1 provides a passage through the housing 10002-1 between the base surface 10004 and the top surface 10008. For example, the top surface 10008 may be in fluid communication with the base surface 10004 through the vent passageway 111-1. More specifically, the vent passageway 111-1 is configured to permit passage of water (e.g., water vapor). In this manner, the housing 10002-1 is configured to pass water (e.g., water vapor) from the base surface 10004 and/or the adhesive patch 106, through the vent inlet 121-1, along the vent passageway 111-1 (e.g., in the distal direction), and through the vent outlet 131-1, and out of the top surface 10008. In this manner, the vent passageway 111-1 may allow the housing 10002-1 to vent moisture from between the housing 10002-1 and the skin. For example, the vent passageway 111-1 may allow ventilation to remove water from the adhesive patch 106.

In some examples, the vent inlet 121-1 may be configured to permit passage of liquid water into the vent passageway 111-1. Thus, liquid water may pass through the vent inlet 121-1, along the vent passageway 111-1, and through the vent outlet 131-1 (e.g., by wicking). For example, this may be achieved at least in part by capillary action along the vent passageway 111-1. Furthermore, the vent passageway 111-1 can include specific material coating to facilitate the moisture egress. Material coatings can have hydrophilic or hydrophobic properties, or a combination of the two. For example, a combination of materials can be used along the inlet 121-1, passageway 111-1, and outlet 131-1 to reduce moisture ingress and increase moisture egress. For example, in all of the embodiments herein, the vent inlet can have a hydrophobic coating to facilitate moisture intake, and the vent outlet can have a hydrophobic coating to prevent moisture ingress.

The fluid connection or open pathway between the environment and the base surface 10004 provided by the vent passageway 111-1 may allow air from the environment to pass in through the vent outlet 131-1, along the vent passageway 111-1 (e.g., in the proximal direction), and to the vent inlet 121-1. The air at the vent inlet 121-1 may remove water from the base surface 10004 and/or the adhesive patch 106 (e.g., by evaporation). The air carrying the water can then be removed from the vent passageway 111-1 through the vent outlet 131-1 (e.g., by diffusion). For example, this airflow can allow water vapor to flow from the vent inlet 121-1 out through the vent outlet 131-1. In addition, this airflow can promote evaporation of liquid water which can then be removed as water vapor. In some examples, the air may also carry droplets of liquid water.

In this arrangement, water can pass along the vent passageway 111-1 to be released from the housing 10002-1. This can provide improved moisture egress compared to relying on the moisture passing along the base surface 10004 to the periphery. In addition, passage of water may be less restricted through the vent passageway 111-1 than along the base surface 10004 due to a lack of air flow between the housing 10002-1 and the skin. Accordingly, the vent passageway 111-1 may allow for improved water egress.

In addition, in some examples, the vent passageway 111-1 may provide a shorter path than passing along the base surface 10004 to a periphery of the housing 10002-1 (especially due to the low profile of the housing 10002-1). This may be particularly beneficial in cases where the vent inlet 121-1 is arranged towards the center of the base surface 10004, since the path along the base surface 10004 may be significantly longer than the path through the height of the housing 10002-1 along the vent passageway 111-1. As a result, in some examples, the length of the vent passageway 111-1 may be less than a distance from the vent inlet 121-1 to the periphery of the base surface 10004. Even if the path is longer through the vent passageway 111-1, this may still be preferable than passing along the base surface 10004 (e.g., due to improved air flow).

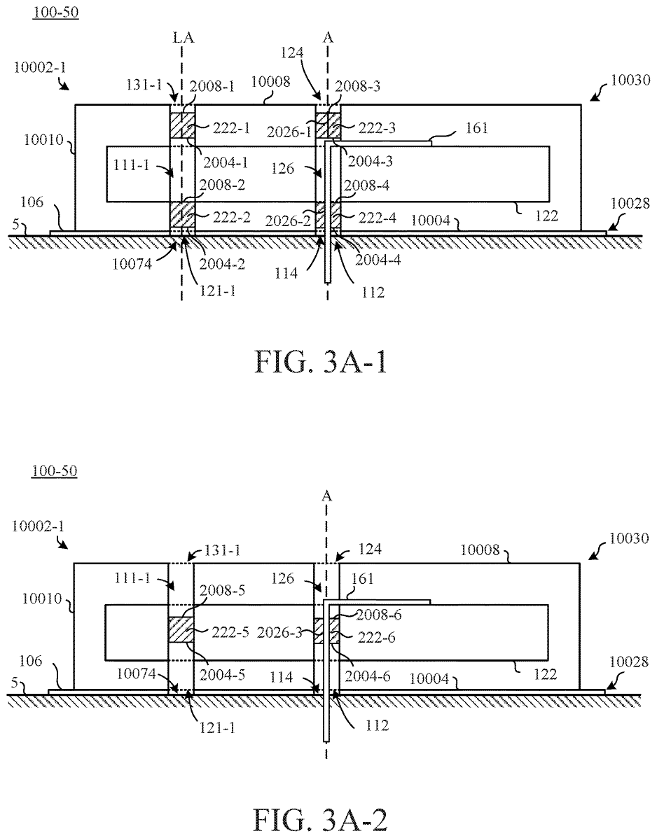

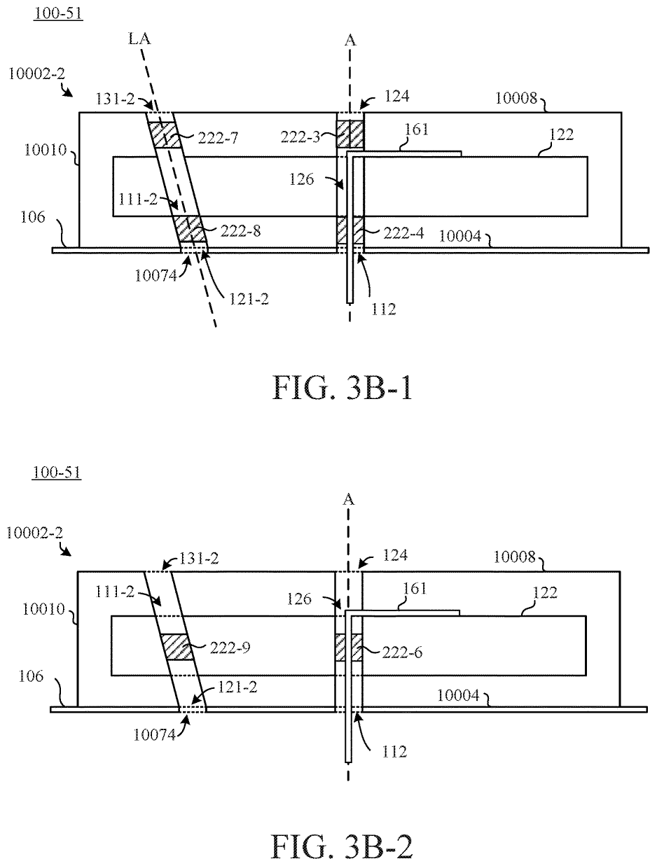

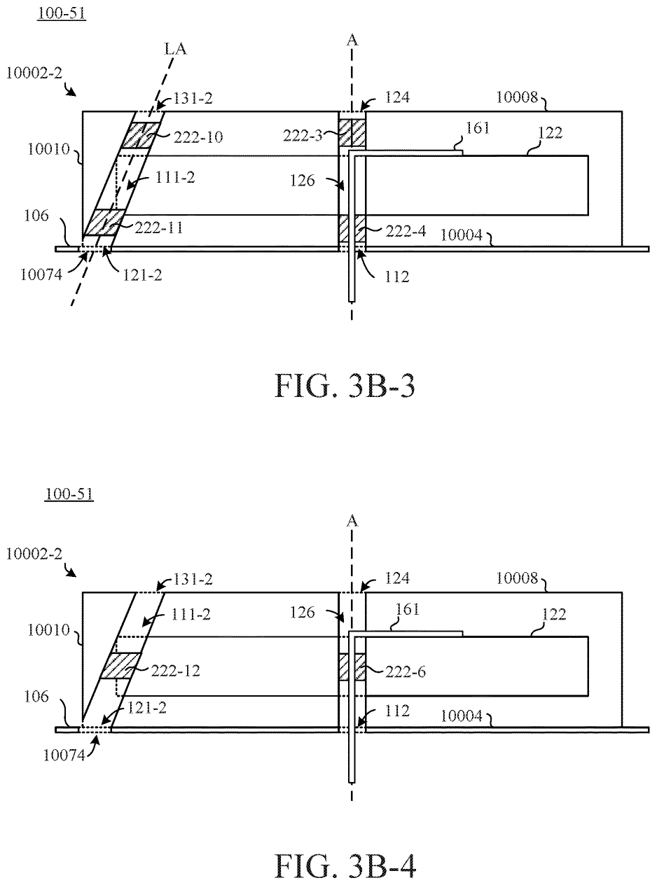

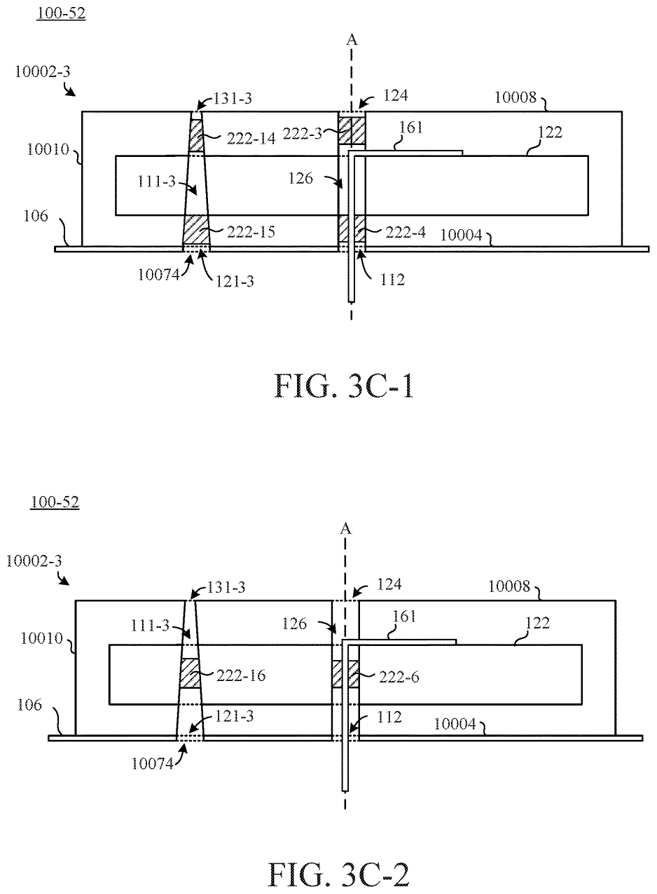

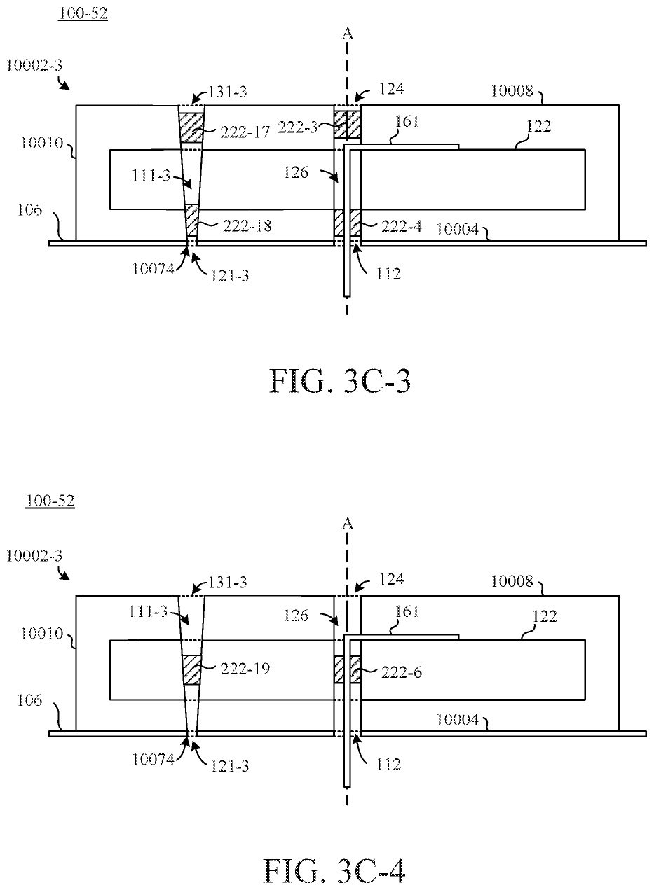

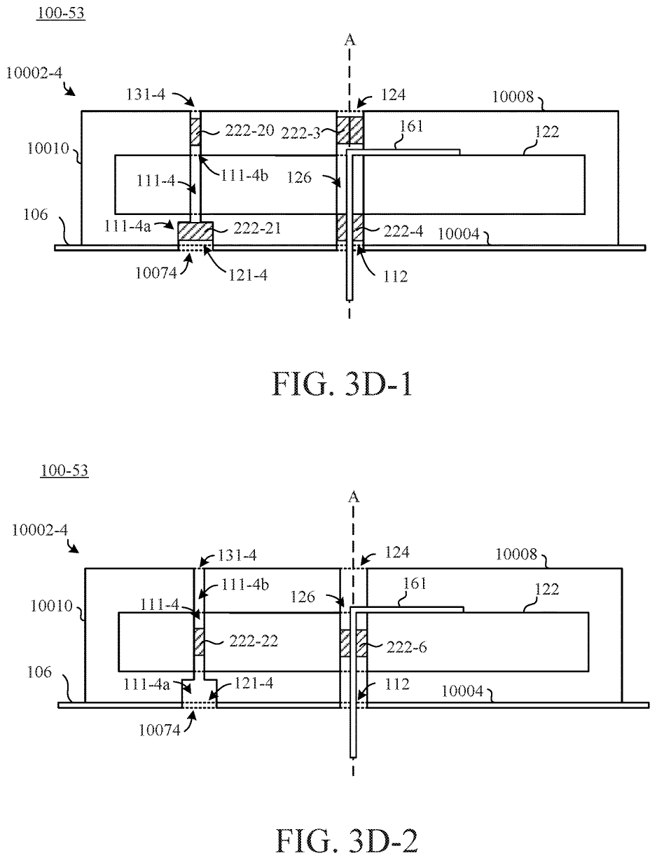

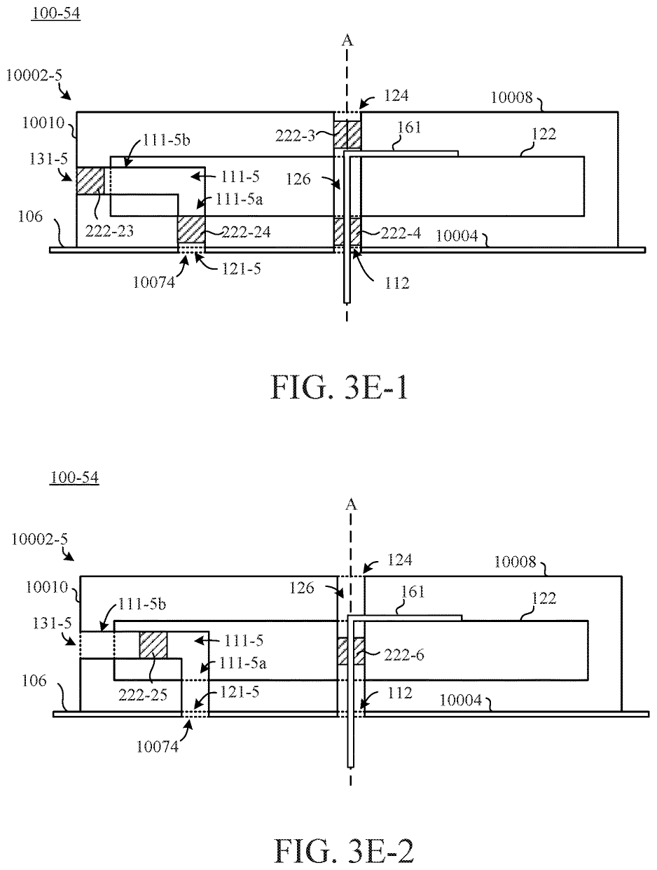

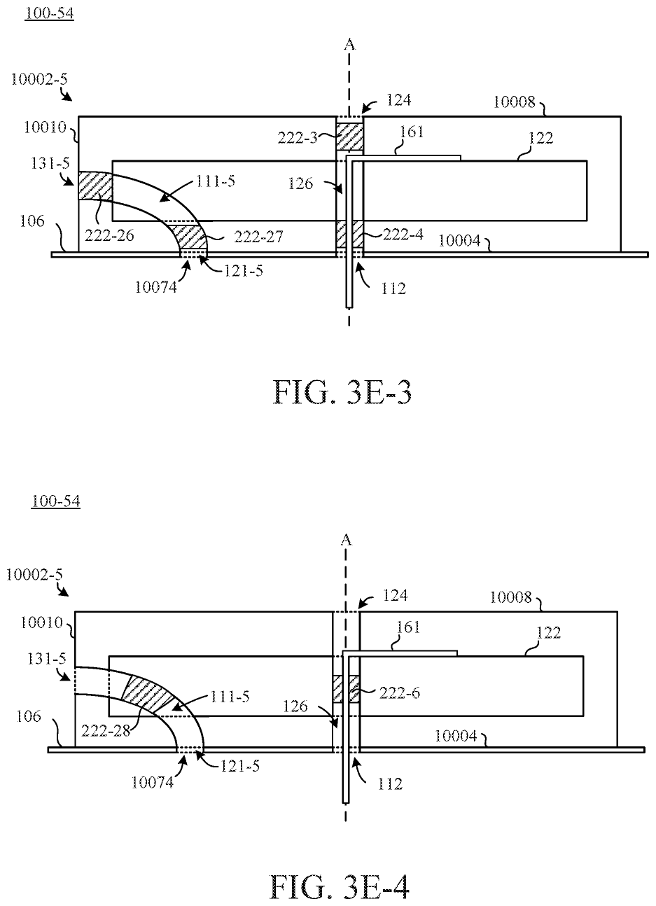

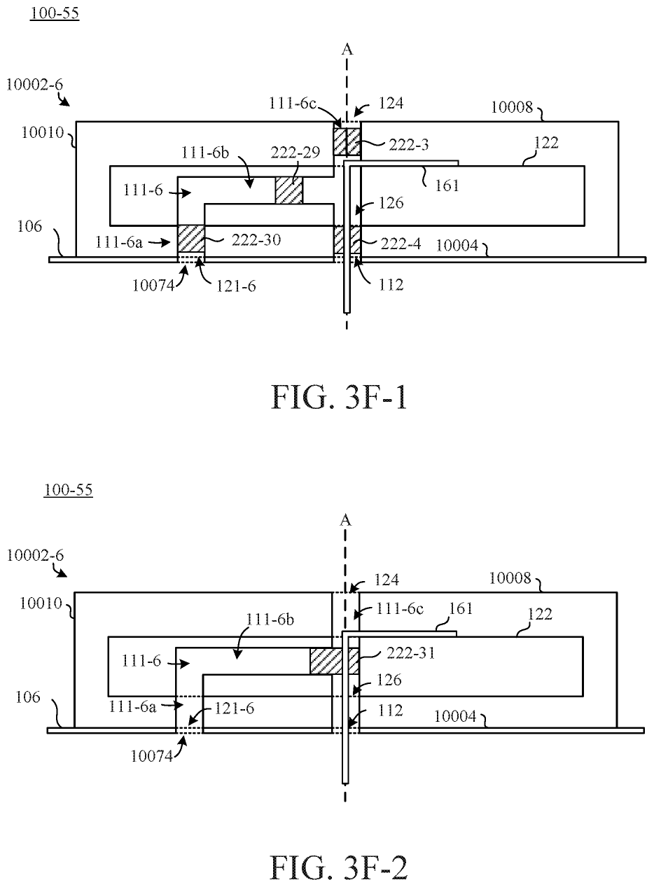

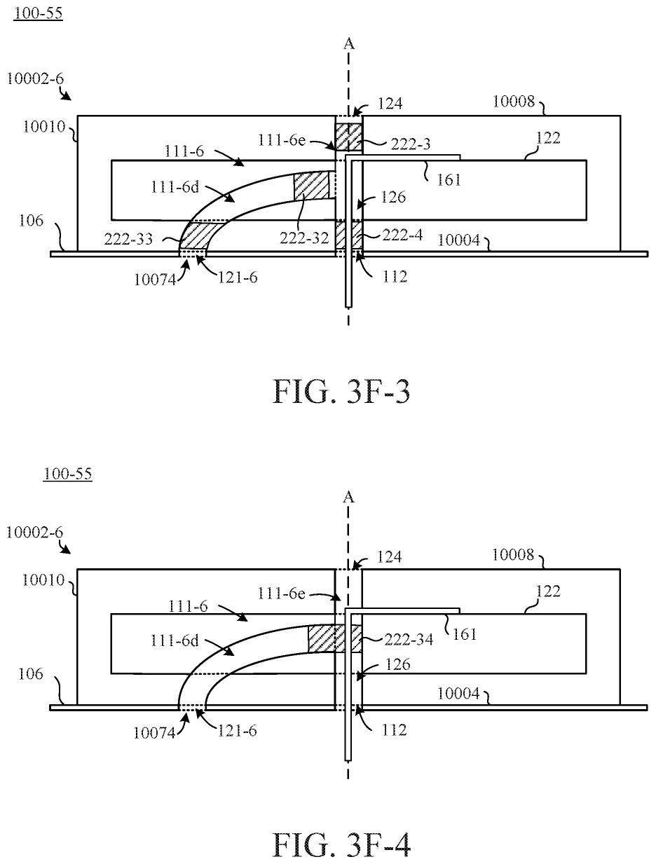

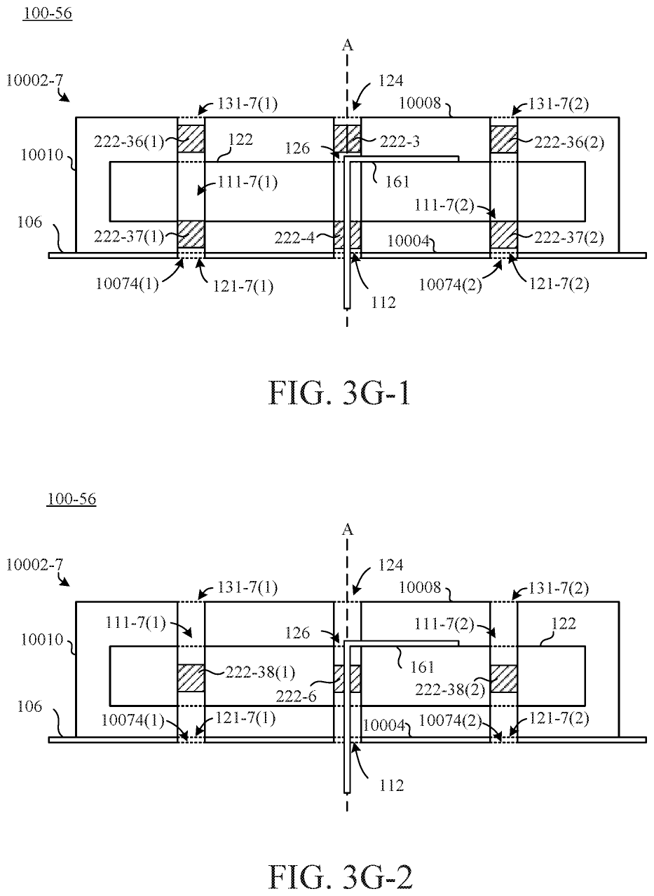

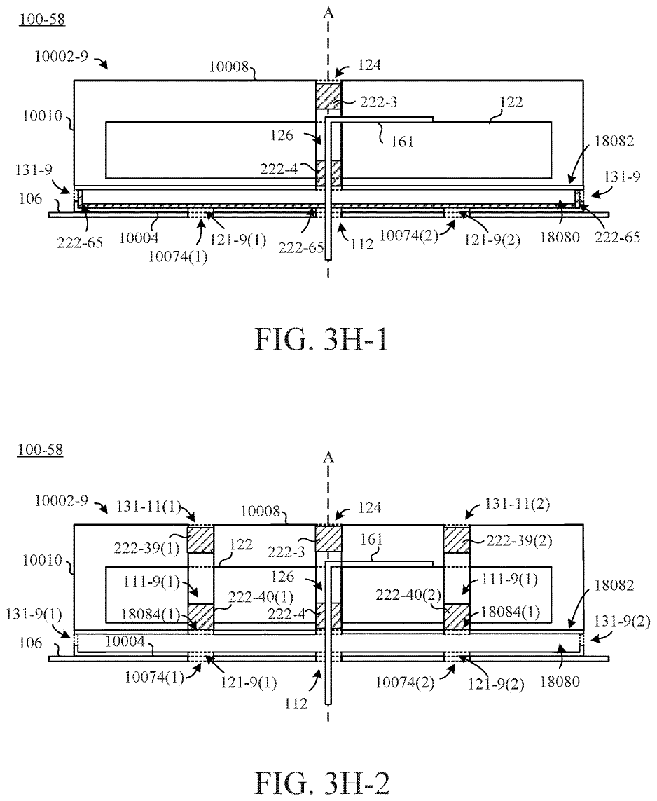

In this embodiment, the vent inlet 121-1 is located at a position on the base surface 10004 spaced away from the periphery of the base surface 10004. For example, the adhesive vent aperture 10074 does not intersect the periphery of the base surface 10004. Thus, the base surface 10004 encircles the adhesive vent aperture 10074 and constitutes a border around the adhesive vent aperture 10074 in two-dimensions (e.g., when an external observer views the base side of the OBD along an axis normal to the base surface 10004, similar to the view of FIG. 2A-3). This is in contrast to the adhesive vent aperture 10074 being located at the periphery of the base surface 10004 (e.g., intersecting the side surface 10010). Since moisture at the edge of the base surface 10004 is more easily able to evaporate, providing the adhesive vent aperture 10074 towards an inner region of the base surface 10004 (e.g., more towards the center) is beneficial to allow release of trapped moisture.

In some examples, the vent passageway 111-1 may be formed from a passageway wall that extends between the base surface 10004 and the top surface 10008, more specifically between the vent inlet 121-1 and the vent outlet 131-1. The passageway wall defines a periphery of the vent passageway 111-1, thereby providing an interior space allowing the water to be passed through the vent passageway 111-1.

As shown in FIG. 2A-1, the vent passageway 111-1 extends through the sensor electronics 122. In particular, the sensor electronics 122 comprises an aperture for receiving the vent passageway 111-1 therethrough. The passageway wall may extend through the aperture. For example, the sensor electronics 122 may comprise a PCB with an aperture for the vent passageway 111-1 to pass through. In other examples, the vent passageway 111-1 may be routed around the sensor electronics 122. Similarly, the sensor passageway 126 may pass through an aperture in the sensor electronics 122 (e.g., through the PCB).

In some examples, the vent passageway 111-1 may be sealed such that the sensor electronics 122 is not exposed to the water passing along the vent passageway 111-1. For example, the passageway wall may seal the sensor electronics 122 from the interior space of the vent passageway 111-1. This can avoid water damage to the sensor electronics 122. In a similar manner, the sensor passageway 126 may be sealed to avoid water coming into contact with the sensor electronics 122. In another example, the interior space of the housing can be filled around the components and sensor electronics 122, such as by molding or potting, and the vent passageway 111-1 may extend through the molding or potting material.

In this embodiment, the vent passageway 111-1 is positioned off-center in the housing 10002-1. In particular, the vent inlet 121-1 is positioned off-center in the base surface 10004 (e.g., arranged towards one side of the base surface 10004). Specifically, the vent inlet 121-1 is arranged between the sensor aperture 112 (aligned with the central axis A) and the periphery of the base surface 10004. The vent outlet 131-1 is positioned off-center in the top surface 10008 (e.g., arranged towards one side of the top surface 10008). Specifically, the vent outlet 131-1 is arranged between the sharp aperture 124 (aligned with the central axis A) and the periphery of the top surface 10008. In this embodiment, the vent inlet 121-1 and the vent outlet 131-1 are aligned in a direction parallel to the central axis A. In this manner, the vent passageway 111-1 is arranged between the sensor passageway 126 and the side surface 10010. In other examples, the vent outlet 131-1 may not be aligned with the vent inlet 121-1 (e.g., see FIGS. 2B-1 and 2B-2).

In some examples, vent passageway 111-1 may be provided at other locations. For example, the vent inlet 121-1 may be equally spaced between the sensor aperture 112 and the periphery of the base surface 10004. In other examples, the vent inlet 121-1 may be closer to the sensor aperture 112 or the periphery of the base surface 10004.

As mentioned above, in this embodiment, the sensor passageway 126 is arranged along the central axis A. In other examples, the sensor passageway 126 may not be aligned with the central axis A. Instead, the sensor passageway 126 may be arranged off-center, such as towards one side of the housing 10002-1.

In some examples, the sensor aperture 112 and the vent inlet 121-1 may be arranged on the same side of the base surface 10004 relative to the central axis A (e.g., in the same half of the circular shape of the base surface 10004). For example, the sensor aperture 112 and the vent inlet 121-1 may be arranged between the central axis A and the periphery of the base surface 10004. In some examples, the sensor aperture 112 and the vent inlet 121-1 may be radially aligned (e.g., aligned along a radius between the central axis A and the periphery of the base surface 10004). In other examples, the sensor aperture 112 and the vent inlet 121-1 are not radially aligned.

In other examples, the sensor aperture 112 may be arranged on an opposite side of the base surface 10004 to the vent inlet 121-1 relative to the central axis A (e.g., in different halves of the circular shape of the base surface 10004). For example, the central axis A may be arranged between the vent inlet 121-1 and the sensor aperture 112.

The vent outlet 131-1 may be positioned in a corresponding manner to the vent inlet 121-1. In such examples, the vent outlet 131-1 may be aligned with the vent inlet 121-1 in a direction parallel to the central axis A. In other examples, the vent outlet 131-1 may not be aligned with the vent inlet 121-1 (e.g., offset).

In other examples, the vent passageway 111-1 may be aligned with the central axis A. For example, the vent inlet 121-1 may be arranged in the center of the base surface 10004. The vent outlet 131-1 may be arranged in the center of the top surface 10008. The vent inlet 121-1 may then be aligned with the vent outlet 131-1 along the central axis A. The sensor passageway 126 may be relocated accordingly. For example, the sensor passageway 126 may be positioned off-center, such as towards one side of the housing 10002-1.

In this embodiment, the vent passageway 111-1 is straight. For example, opposing sides of the passageway wall may be parallel. In particular, the vent passageway 111-1 extends in a direction parallel to the central axis A (e.g., perpendicular to the base surface 10004). A length of the vent passageway 111-1 extends between the vent inlet 121-1 and the vent outlet 131-1. In this embodiment, the length of the vent passageway 111-1 extends in a direction parallel to the central axis A. The length of the vent passageway extends along longitudinal axis LA. Here, the longitudinal axis LA is perpendicular to at least the portion of surface 10004 at and around vent inlet 121-1. The longitudinal axis LA can be normal to at least the portion of surface 10004 at and around vent inlet 121-1. The length of the vent passageway 111-1 may be greater than a width of the vent passageway 111-1. In other examples, the vent passageway 111-1 may not be parallel to the central axis A. For example, vent passageway 111-1 may be angled (e.g., see FIGS. 2B-1 and 2B-2).

In this embodiment, the vent inlet 121-1 has a width (e.g., diameter) of less than 5 mm. In this embodiment, the vent outlet 131-1 has a width (e.g., diameter) of less than 5 mm. For example, the vent inlet 121-1 and/or the vent outlet 131-1 has a width (e.g., diameter) of less than 2 mm, such as less than 1 mm. In one example, the vent inlet 121-1 and/or the vent outlet 131-1 has a width (e.g., diameter) of around 0.5 mm.

In this embodiment, the vent outlet 131-1 is the same size (e.g., width, diameter) as the vent inlet 121-1. In other examples, the vent outlet 131-1 may have a different size to the vent inlet 121-1. In some examples, the vent inlet 121-1 has the same size as the sensor aperture 112. In other examples, the vent inlet 121-1 has a smaller size than the sensor aperture 112 (e.g., since the analyte sensor 10016 does not pass through the vent inlet 121-1). In other examples, the vent inlet 121-1 has a larger size than the sensor aperture 112. Similarly, in some examples, the vent outlet 131-1 has the same size as the sharp aperture 124. In other examples, the vent outlet 131-1 has a smaller size than the sharp aperture 124 (e.g., since the sharp does not pass through the vent outlet 131-1). In other examples, the vent outlet 131-1 has a larger size than the sharp aperture 124.

In this embodiment, the vent passageway 111-1 is cylindrical. In particular, the vent passageway 111-1 is a cylindrical through-hole (e.g., a bore) between the two circular holes defined by the vent inlet 121-1 and the vent outlet 131-1. In this embodiment, the vent passageway 111-1 has a circular cross section in a plane perpendicular to the length of the vent passageway 111-1 (e.g., in a plane perpendicular to the central axis A). In other examples, the cross section may have another shape, such as a square (e.g., forming a vent passageway 111-1 having a cuboid shape).

In this embodiment, the cross section of the vent passageway 111-1 is constant. For example, the cross-sectional size (e.g., measured by two coordinate axes in a cartesian plane) of the vent passageway 111-1 is constant along the length of the vent passageway 111-1. Thus, the width (e.g., diameter) of the vent passageway 111-1 is the same from the vent inlet 121-1 to the vent outlet 131-1. In this manner, the vent passageway 111-1 is cylindrical. In particular, the vent passageway 111-1 is a cylinder with a constant diameter. The vent passageway 111-1 is a straight cylinder (e.g., a right cylinder). The width of the vent passageway 111-1 is the same as the width of the vent inlet 121-1 and the width of the vent outlet 131-1. In other examples, the cross section of the vent passageway 111-1 changes along the length. For example, the vent passageway 111-1 may be tapered (e.g., see FIG. 2C) or have a stepped configuration (e.g., see FIG. 2D).

As mentioned, in this embodiment, the vent outlet 131-1 is in the top surface 10008. In other examples, the vent outlet 131-1 is in a different surface of the housing 10002-1. For example, the vent outlet 131-1 may be in the side surface (e.g., see FIGS. 2E-1 and 2E-2). In another example, the vent outlet 131-1 may be in an intermediate surface. For example, the housing 10002-1 may be similar to the housing 602 or the housing 702 (e.g., see FIGS. 1G and 1H) and the vent outlet 131-1 may be in the intermediate surface (e.g., intermediate surface 632 or intermediate surface 732), in which case the vent outlet 131-1 may be oriented to face in a partially outward direction (e.g., partially facing the distal direction and partially facing outwards). In some examples, the side surface may be omitted and the vent outlet 131-1 may be arranged in the top surface (e.g., top surface 508) connected to the base (e.g., see FIG. 1F), in which case the vent outlet 131-1 may be oriented in a top section or side section of the top surface.

As mentioned, in this embodiment, the adhesive patch 106 comprises an adhesive vent aperture 10074 aligned with the vent inlet 121-1. In other examples, the adhesive patch 106 does not comprise an adhesive vent aperture 10074. For example, the adhesive patch 106 may cover the vent inlet 121-1. Thus, the adhesive patch 106 may be arranged between the vent inlet 121-1 and the skin, when OBD 100-50 is placed on the skin. This may allow water to be removed effectively from the adhesive patch 106. In some examples, the adhesive patch 106 may include a section provided without adhesive on the top side overlapping with the vent inlet 121-1. This may allow the carrier material of the adhesive patch 106 to be directly exposed to the vent inlet 121-1.

As mentioned, in this embodiment, the adhesive patch 106 is attached to the base surface 10004. In other examples, the adhesive patch 106 may be attached to a different surface of the housing 10002-1. For example, the adhesive patch 106 may be attached to the top surface 10008 and/or the side surface 10010 (and/or the intermediate surface, if present). For example, this may be referred to as an overpatch that is placed over OBD 100-50, such as after placement of the housing 10002-1 on the skin. The overpatch may be provided in addition or instead of the adhesive patch 106 on the base surface 10004. The overpatch may contact the skin around the periphery of the housing 10002-1. In some examples, the overpatch does not contact the base surface 10004. As a result, the base surface 10004 may directly contact the skin. In such examples, the vent inlet 121-1 may allow passage of water directly from the skin. Otherwise, this water could migrate to the periphery of the base surface 10004 and pass into the overpatch. The vent outlet 131-1 may be located on a part of the top surface 10008 (or other surface, such as the side surface 10010 or intermediate surface) that is not covered by the overpatch, in order to allow release of the water. In some examples, the overpatch may comprise an aperture aligned with the vent outlet 131-1 to allow such release of water. In other examples, the overpatch is configured to allow passage of water for release of the water from the vent outlet 131-1 through the overpatch.

### On-Body Device 100-51

Referring to FIGS. 2B-1 and 2B-2, OBD 100-51 is shown according to embodiments. OBD 100-51 may be the same as OBD 100-50 of FIGS. 2A-1 to 2A-4, and may include the same features, except as explained below. The alternative features described in relation to OBD 100-50 may similarly be applied to OBD 100-51 in a corresponding manner. In turn, since OBD 100-50 is similar to OBD 100-1, features of OBD 100-1 may be applied to OBD 100-51. Similarly, the features described in relation to OBDs 100-1′ to 100-32 may be applied to OBD 100-51.

In these embodiments, OBD 100-51 is the same as OBD 100-50, except that OBD 100-51 has a vent passageway with a different configuration. Here, OBD 100-51 comprises a housing 10002-2 with a base surface 10004, top surface 10008 and side surface 10010. OBD 100-51 comprises analyte sensor 161. OBD 100-51 comprises an adhesive patch 106. In this embodiment, the housing 10002-2 comprises. The housing 10002-2 comprises sharp aperture 124, sensor aperture 112, and sensor passageway 126 extending therebetween. In other examples, the housing 10002-2 may have different shapes and may be assembled in different ways, as described herein.

The base surface 10004 comprises a vent inlet 121-2, which is similar to the vent inlet 121-1. The adhesive patch 106 comprises adhesive vent aperture 10074. In other examples, an adhesive vent aperture 10074 is not provided. For example, the adhesive patch 11006 may extend to cover the vent inlet 121-2. The housing 10002-2 comprises a vent outlet 131-2, which is similar to the vent outlet 131-1, except as described below. The vent outlet 131-2 is in the top surface 10008. The housing 10002-2 comprises a vent passageway 111-2, which is similar to the vent passageway 111-1, except as described below.

Unlike the vent outlet 131-1, the vent outlet 131-2 is offset from the vent inlet 121-2. In particular, the vent outlet 131-2 is offset from the vent inlet 121-2 relative to a direction parallel to the central axis A (e.g., offset in the lateral dimension, such as in a plane perpendicular to the central axis A). For example, the vent inlet 121-1 is not aligned with the vent outlet 131-1 (e.g., not aligned in a direction parallel to the central axis A). In the embodiment of FIG. 2B-1, the vent outlet 131-2 is closer to the periphery of the top surface 10008 than the vent inlet 121-2 is to the periphery of the base surface 11006. In the embodiment of FIG. 2B-2, the vent inlet 121-2 is at the periphery of the base surface 11006 while the vent outlet 131-2 is spaced apart and relatively farther away from the periphery of the top surface 10008. The vent inlet 121-2 intersects the periphery of the base surface 11006, where the base surface 11006 meets the side surface 10010.

In some examples, an axis passing through the center of the vent inlet 121-2 (e.g., perpendicular to the base surface 10004) is not aligned with an axis passing through the center of the vent outlet 131-2 (e.g., perpendicular to the top surface 10008). In some examples, the vent inlet 121-2 may be fully offset from the vent outlet 131-2 (e.g., the vent inlet 121-2 does not partially overlap the vent outlet 131-2 in a direction parallel to the central axis A). In other examples, the vent inlet 121-2 may be partially offset from the vent outlet 131-2 (e.g., the vent inlet 121-2 partially overlaps the vent outlet 131-2 in a direction parallel to the central axis A).

In this manner, the vent passageway 111-2 extends between the vent inlet 121-2 and the vent outlet 131-2 in a direction that is at an angle to a direction parallel to the central axis A. For example, the vent passageway 111-2 may be angled or sloped. In particular, the vent passageway 111-2 may be arranged at an angle of less than 45° to the central axis A. In the embodiment of FIG. 2B-1, the vent passageway 111-2 extends along a longitudinal axis LA outwardly from vent inlet 121-2 to vent outlet 131-2 (e.g., away from the central axis A, or in a direction towards the periphery of the housing 10002-2). In the embodiment of FIG. 2B-2, the vent passageway 111-2 extends along a longitudinal axis LA inwardly from vent inlet 121-2 to vent outlet 131-2 (e.g., towards the central axis A, or in a direction away from the periphery of the housing 10002-2).

In other examples, the vent passageway 111-2 may be curved or may comprise corners or bends to allow the vent outlet 131-2 to be positioned at a different location (e.g. offset) to the vent inlet 121-2.

This arrangement may allow the vent passageway 111-2 to be routed through the housing 10002-2 in a more convenient manner. For example, the vent passageway 111-2 may be routed through the sensor electronics 122 at the desired location (not limited to being aligned with the vent inlet 121-2). In one example, the vent inlet 121-2 may be positioned towards the center of the base surface 10004 (since accumulation of moisture may be a more significant problem in this location compared to towards the periphery) while the vent passageway 111-2 may extend through the sensor electronics 122 at a position avoiding certain components (e.g., a large battery).

In this embodiment, the vent passageway 111-2 is positioned off-center in the housing 10002-2. In particular, the vent inlet 121-2 is positioned off-center in the base surface 10004 and the vent outlet 131-2 is positioned off-center in the top surface 10008. In other examples, the vent inlet 121-2 or the vent outlet 131-2 may be aligned with the central axis A. The vent passageway 111-2 may have various positions as explained in relation to the vent passageway 111-1.

In this embodiment, the width (e.g., diameter) of the vent passageway 111-2 is the same along the length of the vent passageway 111-2 from the vent inlet 121-2 to the vent outlet 131-2. The width of the vent inlet 121-2 is the same as the width of the vent outlet 131-2. Thus, the width of the vent passageway 111-2 is the same as the width of the vent inlet 121-2 and the width of the vent outlet 131-2. In other examples, the width of the vent passageway 111-2 changes along the length. In other examples, the width of the vent inlet 121-2 is different to the vent outlet 131-2.

In this embodiment, the vent passageway 111-2 is cylindrical. The vent inlet 121-2 and the vent outlet 131-2 are circular. The vent passageway 111-2 has a circular cross section perpendicular to the length. Unlike the vent passageway 111-1, which is a straight cylinder (e.g., a right cylinder) extending in a direction parallel to the central axis A, the vent passageway 111-2 is an angled cylinder (e.g., an oblique cylinder) extending at an angle to a direction parallel to the central axis A. In some examples, the vent passageway 111-2 is oblique. In this embodiment, the vent passageway 111-2 is straight and angled.

The vent passageway 111-2 is not limited to being cylindrical. For example, the vent passageway 111-2 may be angled (e.g., the vent inlet 121-2 and the vent outlet 131-2 may be offset from each other) whilst having a different shape. For example, the cross-sectional shape (e.g., measured by two coordinate axes in a cartesian plane) of the vent passageway 111-2 may be a square or other shape. In some examples, the vent inlet 121-2 and the vent outlet 131-2 may be different shapes to each other. In other examples, the cross section of the vent passageway 111-2 may also change along the length of the vent passageway 111-2. For example, the vent passageway 111-2 may be tapered (e.g., see FIG. 2C) or have a stepped configuration (e.g., see FIG. 2D).

As mentioned, in these embodiments, the vent outlet 131-2 is in the top surface 10008. In other examples, the vent outlet 131-2 is in a different surface of the housing 10002-2. For example, the vent outlet 131-2 may be in the side surface 10010 (e.g., see FIGS. 2E-1 and 2E-2). In another example, the vent outlet 131-2 may be in an intermediate surface.

### On-Body Device 100-52

Referring to FIGS. 2C-1 and 2C-2, OBD 100-52 is shown according to embodiments. OBD 100-52 may be the same as OBD 100-50 of FIGS. 2A-1 to 2A-4, and may include the same features, except as explained below. The alternative features described in relation to OBD 100-50 may similarly be applied to OBD 100-52 in a corresponding manner. In turn, since OBD 100-50 is similar to OBD 100-1, features of OBD 100-1 may be applied to OBD 100-52. Similarly, the features described in relation to OBDs 100-1′ to 100-32 may be applied to OBD 100-52. Similarly, the features described in relation to OBD 100-51 may be applied to OBD 100-52.

In this embodiment, OBD 100-52 is the same as OBD 100-50, except that OBD 100-52 has a vent passageway with a different configuration. In this embodiment, OBD 100-52 comprises a housing 10002-3 with base surface 10004, top surface 10008 and side surface 10010. OBD 100-52 comprises analyte sensor 161 and adhesive patch 106. The housing 10002-3 comprises sharp aperture 124, sensor aperture 112, and sensor passageway 126 extending therebetween. In other examples, the housing 10002-3 may have different shapes and may be assembled in different ways, as described herein.

In this embodiment, the base surface 10004 comprises a vent inlet 121-3, which is similar to the vent inlet 121-1. The adhesive patch 106 comprises an adhesive vent aperture 10074. In other examples, an adhesive vent aperture 10074 is not provided. For example, the adhesive patch 106 may extend to cover the vent inlet 121-3. The housing 10002-3 comprises a vent outlet 131-3, which is similar to the vent outlet 131-1, except as described below. The vent outlet 131-3 is in the top surface 10008. The housing 10002-3 comprises a vent passageway 111-3, which is similar to the vent passageway 111-1, except as described below.

Unlike the vent outlet 131-1, the vent outlet 131-3 has a different size to the vent inlet 121-3. In the embodiment of FIG. 2C-1, the vent outlet 131-3 is smaller than the vent inlet 121-3. More specifically, the vent outlet 131-3 has a width (e.g., a diameter) that is smaller than a width (e.g., a diameter) of the vent inlet 121-3. The larger vent inlet 121-3 may allow collection of water from a larger area, which may provide for improved water egress. The smaller vent outlet 131-3 may reduce the amount of water (e.g., liquid water) that may enter the vent outlet 131-3 from the environment.

In embodiments of FIGS. 2C-1 and 2C-2, the vent passageway 111-3 has a cross section that changes along the length of the vent passageway 111-3. In particular, the cross-sectional size (e.g., measured by two coordinate axes in a cartesian plane) of the vent passageway 111-3 changes along the length. For example, opposing sides of the passageway wall may be non-parallel (e.g., diverging or converging). In particular, the width (e.g., the diameter) of the vent passageway 111-3 changes at a constant or variable rate along at least a portion of the length of the vent passageway 111-3, such that the vent passageway has a tapered width portion. In the embodiment of FIG. 2C-1, the width decreases from the vent inlet 121-3 to the vent outlet 131-3. In particular, the width decreases at a constant rate along at least a portion of the length, in this example the entire length, of the vent passageway 111-3. In cross-section, the passageway wall has straight and angled sides. In this manner, the vent passageway 111-3 has a tapered configuration.

In other examples, the cross-sectional size of the vent passageway 111-3 increases from the vent inlet 121-3 to the vent outlet 131-3. In the embodiment of FIG. 2C-2, the width decreases from the vent inlet 121-3 to the vent outlet 131-3. In particular, the width decreases at a constant rate along a portion of the length, in this example the entire length, of the vent passageway 111-3. In cross-section, the passageway wall has straight and angled sides. In this manner, the vent passageway 111-3 has a tapered configuration. In some examples, the vent inlet 121-3 may be smaller than the vent outlet 131-3. In the embodiment of FIG. 2C-2, the vent inlet 121-3 has a width (e.g., a diameter) that is smaller than a width (e.g., a diameter) of the vent outlet 131-3. For example, the larger vent outlet 131-3 may allow release of water from a larger area, which may provide for improved water egress. The smaller vent inlet 121-3 may maximize the surface area of the base surface 10004 in contact with the adhesive patch 106, which may improve the retention of the housing 10002-3 on the skin.

In other examples, the cross-sectional size of the vent passageway 111-3 changes at a non-constant rate. In cross-section, the passageway wall may have curved sides. For example, the curvature may increase or decrease along the length of the vent passageway 111-3.

In this embodiment, the vent passageway 111-3 is positioned off-center in the housing 10002-3. In particular, the vent inlet 121-3 is positioned off-center in the base surface 10004 and the vent outlet 131-3 is positioned off-center in the top surface 10008. In other examples, the vent inlet 121-3 and/or the vent outlet 131-3 may be aligned with the central axis A. For example, the vent passageway 111-3 may be aligned with the central axis A. The vent passageway 111-3 may have various positions as explained in relation to the vent passageway 111-1.

In this embodiment, the vent passageway 111-3 is parallel to the central axis A. In other examples, the vent passageway 111-3 may not be parallel to the central axis A. In particular, the vent inlet 121-3 may be offset relative to the vent outlet 131-3. For example, the vent passageway 111-3 may be angled (e.g., see FIGS. 2B-1 and 2B-2). In other examples, the vent passageway 111-3 may be curved or may comprise corners or bends to allow the vent outlet 131-3 to be positioned at a different location (e.g. offset) to the vent inlet 121-3.

In this embodiment, the cross-sectional shape of the vent passageway 111-3 is circular. The vent inlet 121-3 and the vent outlet 131-3 are circular. Since the width (diameter in this case) decreases, the vent passageway 111-3 comprises a conical shape (e.g., compared to the cylindrical shape of the vent passageway 111-1). More specifically, the vent passageway 111-3 may be a frustoconical shape (e.g., a truncated cone), since the vent passageway 111-3 terminates at the vent outlet 131-3 having a defined width, rather than at a point (being an apex of a cone).

The tapered vent passageway 111-3 is not limited to being conical. In other examples, the cross-sectional shape may be different. For example, the cross-sectional shape of the vent passageway 111-3 may be a square or other shape. In this case, the vent passageway 111-3 may be pyramidal. More specifically, the vent passageway 111-3 may be a frustum shape (e.g., a square frustum), which may be referred to as a frusto-pyramidal shape (e.g., a truncated pyramid). In some examples, the vent inlet 121-3 and the vent outlet 131-3 may be different shapes to each other.

In other examples, the cross-sectional size of the vent passageway 111-3 decreases and then increases along the length, or vice versa. For example, a first section of the vent passageway 111-3 may have a decreasing width and a second section of the vent passageway 111-3 may have an increasing width (e.g., from the vent inlet 121-3 to the vent outlet 131-3). For example, the vent passageway 111-3 may have an hourglass shape (e.g., with straight or curved sections with changing width). For example, the vent passageway 111-3 may have a hyperboloid shape. This may allow the vent inlet 121-3 and/or the vent outlet 131-3 to have a larger width than at least a portion of the vent passageway 111-3. For example, the vent passageway 111-3 may have a smaller width (e.g., smallest width) at a point where it passes through the sensor electronics 122 (e.g., through an aperture in the PCB), minimizing the impact on the sensor electronics 122. For example, the vent inlet 121-3 and the vent outlet 131-3 may have the same width, but the vent passageway 111-3 may change width (e.g., decreasing and then increasing, or vice versa) therebetween.

As mentioned, in this embodiment, the vent outlet 131-3 is in the top surface 10008. In other examples, the vent outlet 131-3 is in a different surface of the housing 10002-3. For example, the vent outlet 131-3 may be in the side surface (e.g., see FIGS. 2E-1 and 2E-2). In another example, the vent outlet 131-3 may be in an intermediate surface.

### On-Body Device 100-53

Referring to FIG. 2D, an OBD 100-53 is shown according to an embodiment. OBD 100-53 may be the same as OBD 100-50 of FIGS. 2A-1 to 2A-4, and may include the same features, except as explained below. Various alternative features described in relation to OBD 100-50 may similarly be applied to OBD 100-53 in a corresponding manner. In turn, since OBD 100-50 is similar to OBD 100-1, features of OBD 100-1 may be applied to OBD 100-53. Similarly, the features described in relation to OBDs 100-1′ to 100-32 may be applied to OBD 100-53. Similarly, the features described in relation to OBD 100-51 or 12000 may be applied to OBD 100-53.

In this embodiment, OBD 100-53 is the same as OBD 100-50, except that OBD 100-53 has a vent passageway with a different configuration. In this embodiment, OBD 100-53 comprises a housing 10002-4 with a base surface 10004, which is similar to the base surface 10004, except as described below. OBD 100-53 comprises analyte sensor 161. OBD 100-53 comprises adhesive patch 106. In this embodiment, the housing 10002-4 with a base surface 10004, top surface 10008 and side surface 10010. The housing 10002-4 comprises sharp aperture 124, sensor aperture 112, and sensor passageway 126 extending therebetween. In other examples, the housing 10002-4 may have different shapes and may be assembled in different ways, as described herein.

In this embodiment, the base surface 10004 comprises a vent inlet 121-4, which is similar to the vent inlet 121-1, except as described below. The adhesive patch 106 comprises an adhesive vent aperture 10074. In other examples, an adhesive vent aperture 10074 is not provided. For example, the adhesive patch 106 may extend to cover the vent inlet 121-4. The housing 10002-4 comprises a vent outlet 131-4, which is similar to the vent outlet 131-1, except as described below. The vent outlet 131-4 is in the top surface 10008. The housing 10002-4 comprises a vent passageway 111-4, which is similar to the vent passageway 111-1, except as described below.

Similarly to the housing 10002-3, the housing 10002-4 has a vent outlet 131-4 with a different size to the vent inlet 121-4. In particular, the vent inlet 121-4 has a larger width (e.g., diameter) than the vent outlet 131-4. Unlike the tapered vent passageway 111-3, the vent passageway 111-4 has a stepped configuration. In particular, the vent passageway 111-4 comprises a first section 111-4a and a second section 111-4b. The first section 111-4a extends from the vent inlet 121-4 and connects to the second section 111-4b. The second section 111-4b extends from the vent outlet 131-4 and connects to the first section 111-4a. Thus, the first section 111-4a is more proximal compared to the second section 111-4b.

In this embodiment, the first section 111-4a has a larger width (e.g., diameter) than the second section 111-4b. This may allow the vent inlet 121-4 to be wider whilst a part of the vent passageway 111-4 (the second section 111-4b) is narrower. The change in the width of the vent passageway 111-4 can be abrupt or gradual. In this embodiment, the first section 111-4a has a constant width and the second section 111-4b has a constant width which is different to the width of the first section 111-4a (e.g., larger) with a step transition in width that is generally abrupt. Thus, the width of the vent passageway 111-4 changes by a discrete amount between the first section 111-4a and the second section 111-4b. The first section 111-4a and the second section 111-4b each have passageway wall sections that have straight and parallel opposing sides. In other examples, the change in width may be more gradual, such as providing an angled or curved surface between the first section 111-4a and the second section 111-4b.

In other examples, the first section 111-4a may have a smaller width (e.g., diameter) than the second section 111-4b. In some examples, the vent inlet 121-4 may be smaller than the vent outlet 131-4. More specifically, the vent inlet 121-4 has a width (e.g., a diameter) that is smaller than a width (e.g., a diameter) of the vent outlet 131-4.

In some examples, the first section 111-4a may be arranged proximally of (e.g., below) the sensor electronics 122 (e.g., proximally of a PCB). For example, the wider section of the vent passageway 111-4 (e.g., the first section 111-4a) may be arranged between the base surface 10004 and the sensor electronics 122, whilst the vent passageway 111-4 can be routed through the sensor electronics 122 through a hole smaller than the size of the vent inlet 121-4 (e.g., the second section 111-4b may extend through the sensor electronics 122). This allows for the combination of a large area for collection of moisture while minimizing the impact on the sensor electronics 122.

In this embodiment, the length of the first section 111-4a is less than the length of the second section 111-4b. In other examples, the first section 111-4a may be longer than the second section 111-4b, or the lengths may be the same.

In this embodiment, the vent passageway 111-4 is positioned off-center in the housing 10002-4. In particular, the vent inlet 121-4 is positioned off-center in the base surface 10004 and the vent outlet 131-4 is positioned off-center in the top surface 10008. In other examples, the vent inlet 121-4 and/or the vent outlet 131-4 may be aligned with the central axis A. For example, the vent passageway 111-4 may be aligned with the central axis A. The vent passageway 111-4 may have various positions as explained in relation to the vent passageway 111-1.

In this embodiment, the vent passageway 111-4 is parallel to the central axis A. In other examples, the vent passageway 111-4 may not be parallel to the central axis A. In particular, the vent inlet 121-4 may be offset relative to the vent outlet 131-4. For example, the vent passageway 111-4 may be angled (e.g., see FIGS. 2B-1 and 2B-2). In other examples, the vent passageway 111-4 may be curved or may comprise corners or bends to allow the vent outlet 131-4 to be positioned at a different location (e.g. offset) to the vent inlet 121-4.

In this embodiment, the vent inlet 121-4 and the vent outlet 131-4 are circular, and the first section 111-4a and the second section 111-4b have a circular cross section. The first section 111-4a has a cylindrical shape having a first width (in this case a diameter). The first width may be equal to the width of the vent inlet 121-4. The second section 111-4b has a cylindrical shape having a second width (in this case a diameter). The second width may be equal to the width of the vent outlet 131-4. In this embodiment, the second width is less than the first width. In other examples, the second width may be larger than the first width.

The stepped vent passageway 111-4 is not limited to having cylindrical-shaped sections. In other examples, the cross-sectional shape may be different. For example, the cross-sectional shape of the first section 111-4a and/or the second section 111-4b may be a square or other shape. The cross-sectional shape of the first section 111-4a may be the same or different to the cross-sectional shape of the second section 111-4b.

In some examples, the vent passageway 111-4 may comprise at least one further section. For example, the vent passageway 111-4 may comprise three or more sections. In one example, the vent passageway 111-4 may comprise a first section connected to the vent inlet 121-4 and a second section connected to the vent outlet 131-4. The vent passageway 111-4 may also have a third section between the first section and the second section. For example, the third section may have a smaller cross-sectional area than the first and second sections, for routing through the sensor electronics 122. For example, the vent passageway 111-4 may have a smaller width (e.g., smallest width) at a point (e.g., the third section) where it passes through the sensor electronics 122 (e.g., through an aperture in the PCB), minimizing the impact on the sensor electronics 122. In this example, the first and second sections may have the same size (e.g., the vent inlet 121-4 and the vent outlet 131-4 may be the same size, such as in width or diameter). In other examples, the first and second sections may have a different size. In other examples, more than three sections may be provided (e.g., four or more).

In other examples, one or more sections of the vent passageway 111-4 may be angled (e.g., see FIGS. 2B-1 and 2B-2). For example, the first section 111-4a may be angled and the second section 111-4b may not be angled (e.g., may be parallel to the central axis A). In other examples, one or more sections of the vent passageway 111-4 may be curved or the vent passageway 111-4 may comprise corners or bends.

In other examples, one or more sections of the vent passageway 111-4 may have a changing width along the length of the section. For example, one or more sections of the vent passageway 111-4 may be tapered (e.g., see FIG. 2C). In other examples, the cross-sectional size (e.g., measured by two coordinate axes in a cartesian plane) of one or more sections of the vent passageway 111-4 changes at a non-constant rate. In cross-section, the passageway wall section may have curved sides. For example, the curvature may increase or decrease along the length of the section of the vent passageway 111-4.

As mentioned, in this embodiment, the vent outlet 131-4 is in the top surface 10008. In other examples, the vent outlet 131-4 is in a different surface of the housing 10002-4. For example, the vent outlet 131-4 may be in the side surface (e.g., see FIGS. 2E-1 and 2E-2). In another example, the vent outlet 131-4 may be in an intermediate surface.

### On-Body Device 100-54

Referring to FIGS. 2E-1 and 2E-2, an OBD 100-54 is shown according to an embodiment. OBD 100-54 may be the same as OBD 100-50 of FIGS. 2A-1 to 2A-4, and may include the same features, except as explained below. Various alternative features described in relation to OBD 100-50 may similarly be applied to OBD 100-54 in a corresponding manner. In turn, since OBD 100-50 is similar to OBD 100-1, features of OBD 100-1 may be applied to OBD 100-54. Similarly, the features described in relation to OBDs 100-1′ to 100-32 may be applied to OBD 100-54. Similarly, the features described in relation to OBDs 100-51 to 100-53 may be applied to OBD 100-54.

In these embodiments, OBD 100-54 is the same as OBD 100-50, except that OBD 100-54 has a vent passageway with a different configuration. OBD 100-54 comprises a housing 10002-5 with base surface 10004, top surface 10008 and side surface 10010. OBD 100-54 comprises analyte sensor 161. OBD 100-54 comprises adhesive patch 106. The housing 10002-5 comprises sharp aperture 124, sensor aperture 112, and sensor passageway 126 extending therebetween. In other examples, the housing 10002-5 may have different shapes and may be assembled in different ways, as described herein.

In this embodiment, the base surface 10004 comprises a vent inlet 121-5, which is similar to the vent inlet 121-1. The adhesive patch 106 comprises adhesive vent aperture 10074. In other examples, an adhesive vent aperture 10074 is not provided. For example, the adhesive patch 106 may extend to cover the vent inlet 121-5. The housing 10002-5 comprises a vent outlet 131-5, which is similar to the vent outlet 131-1, except as described below. The housing 10002-5 comprises a vent passageway 111-5, which is similar to the vent passageway 111-1, except as described below.

Unlike the vent outlet 131-1, the vent outlet 131-5 is arranged in the side surface 10010 instead of in the top surface 10008. In particular, the vent outlet 131-5 is an aperture or an opening in the side surface 10010. The top surface 10008 does not have a vent outlet 131-5.

In this manner, the vent outlet 131-5 is not aligned with the vent inlet 121-5 in a direction parallel to the central axis A. Instead, referring first to the embodiment of FIG. 2E-1, the vent passageway 111-5 comprises a first section 111-5a and a second section 111-5b. The first section 111-5a extends from the vent inlet 121-5 and connects to the second section 111-5b. The second section 111-5b extends from the vent outlet 131-5 and connects to the first section 111-5a. In this embodiment, the first section 111-5a extends in a direction parallel to the central axis A. The second section 111-5b extends in a direction perpendicular to the central axis A. The second section 111-5b extends away from the central axis A. The second section 111-5b extends towards the side surface 10010. Thus, the second section 111-5b is perpendicular to the first section 111-5a. In other examples, the angle between the second section 111-5b and the first section 111-5a may not be perpendicular (for example, the angle may be acute or obtuse, such as between 0° and 180°). In this embodiment, the vent passageway 111-5 comprises a corner joining the first section 111-5a to the second section 111-5b. In other examples, the first section 111-5a may be connected to the second section 111-5b by a bend or a curve.

In this arrangement, the water from the base surface 10004 and/or the adhesive patch 106 can pass through the vent inlet 121-5 and into the vent passageway 111-5, through which it can pass to the vent outlet 131-5. More specifically, water can pass from the vent inlet 121-5 into the first section 111-5a and then into the second section 111-5b for release through the vent outlet 131-5. This allows the water to be passed out of the housing 10002-5 via the side surface 10010. This may be particularly useful since it avoids routing the vent passageway 111-5 through the entire thickness of the housing 10002-5 to the top surface 10008. As a result, this can reduce interference with the sensor electronics 122. This arrangement may be particularly useful when providing the vent inlet 121-5 towards the periphery of the base surface 10004, since this minimizes the length of the vent passageway 111-5.

In some examples, the vent passageway 111-5 may be arranged so that the second section 111-5b extends proximally of (e.g., beneath) the sensor electronics 122 (e.g., proximally of a PCB). In this manner, the vent passageway 111-5 may not extend through the sensor electronics 122, which can increase the space available for the sensor electronics 122. The vent outlet 131-5 may be positioned in the side surface 10010 at a position closer to the base surface 10004 than to the top surface 10008 (e.g., towards the proximal side).

In this embodiment, the vent passageway 111-5 is positioned off-center in the housing 10002-5. In particular, the vent inlet 121-5 is positioned off-center in the base surface 10004. The vent outlet 131-5 is positioned on the side surface 10010 which is not aligned with the central axis A. In other examples, the vent inlet 121-5 may be aligned with the central axis A. For example, the first section 111-5a of the vent passageway 111-5 may be aligned with the central axis A. The vent passageway 111-5 may have various positions as explained in relation to the vent passageway 111-1.

In this embodiment, the first section 111-5a of the vent passageway 111-5 is parallel to the central axis A. In other examples, the first section 111-5a may not be parallel to the central axis A. For example, the first section 111-5a may be angled (e.g., see FIGS. 2B-1 and 2B-2). In other examples, the first section 111-5a may be curved or may comprise corners or bends.

In this embodiment, the vent inlet 121-5 and the vent outlet 131-5 are circular, and the first section 111-5a and the second section 111-5b have a circular cross section. The first section 111-5a has a cylindrical shape having a first width (in this case a diameter). The first width may be equal to the width of the vent inlet 121-5. The second section 111-5b has a cylindrical shape having a second width (in this case a diameter). The second width may be equal to the width of the vent outlet 131-5. In this embodiment, the second width is the same as the first width. In other examples, the widths may be different.

The vent passageway 111-5 is not limited to having cylindrical-shaped sections. In other examples, the cross-sectional shape may be different. For example, the cross-sectional shape of the first section 111-5a and/or the second section 111-5b may be a square or other shape. The cross-sectional shape of the first section 111-5a may be the same or different to the cross-sectional shape of the second section 111-5b.

In some examples, the vent passageway 111-5 may comprise at least one further section. For example, the vent passageway 111-5 may comprise three or more sections. In other examples, more than three sections may be provided (e.g., four or more).

In other examples, one or more sections of the vent passageway 111-5 may be angled (e.g., see FIGS. 2B-1 and 2B-2). For example, the first section 111-5a may be angled and the second section 111-5b may not be angled (e.g., may be perpendicular to the central axis A). In other examples, one or more sections of the vent passageway 111-5 may be curved. In the embodiment of FIG. 2E-2, the length of passageway 111-5 is curved from vent inlet 121-5 to vent outlet 131-5. Stated differently, the length of passageway 111-5 extends along a curved longitudinal axis LA.

Referring back to FIG. 2E-1, the first section 111-5a has the same width as the second section 111-5b. In particular, the vent inlet 121-5 has the same size as the vent outlet 131-5. In other examples, the first section 111-5a may have a different width (e.g., diameter) to the second section 111-5b. The vent inlet 121-5 may have a different size to the vent outlet 131-5 (e.g., larger or smaller). For example, the first section 111-5a may have a larger width (or smaller) than the second section 111-5b, such as in a stepped configuration (e.g., see FIG. 2D).

In other examples, one or more sections of the vent passageway 111-5 may have a changing width along the length of the section. For example, one or more sections of the vent passageway 111-5 may be tapered (e.g., see FIG. 2C). In other examples, the cross-sectional size of one or more sections of the vent passageway 111-5 changes at a non-constant rate. In cross-section, the passageway wall section may have curved sides. For example, the curvature may increase or decrease along the length of the section of the vent passageway 111-5.

As mentioned, in this embodiment, the vent outlet 131-5 is in the side surface 10010. In other examples, the vent outlet 131-5 is in a different surface of the housing 10002-5. For example, the vent outlet 131-5 may be in an intermediate surface. In some examples, the vent outlet 131-5 may be in a side section of the top surface 10008. For example, where the top surface 10008 is connected to the base surface 10004 (e.g., see FIG. 1F), the vent outlet 131-5 may be arranged in a side section of the top surface 10008 (e.g., at a point closer to the connection to the base surface 10004 than to the central axis A).

### On-Body Device 100-55

Referring to FIGS. 2F-1 and 2F-2, OBD 100-55 is shown according to embodiments. OBD 100-55 may be the same as OBD 100-50 of FIGS. 2A-1 to 2A-4, and may include the same features, except as explained below. Various alternative features described in relation to OBD 100-50 may similarly be applied to OBD 100-55 in a corresponding manner. In turn, since OBD 100-50 is similar to OBD 100-1, features of OBD 100-1 may be applied to OBD 100-55. Similarly, the features described in relation to OBDs 100-1′ to 100-32 may be applied to OBD 100-55. Similarly, the features described in relation to OBDs 100-51 to 100-54 may be applied to OBD 100-55.

In these embodiments, OBD 100-55 is the same as OBD 100-50, except that OBD 100-55 has a vent passageway with a different configuration. In this embodiment, OBD 100-55 comprises a housing 10002-6 with base surface 10004, top surface 10008 and side surface 10010. OBD 100-55 comprises analyte sensor 161. OBD 100-55 comprises adhesive patch 106. In other examples, the housing 10002-6 may have different shapes and may be assembled in different ways, as described herein.

The base surface 10004 comprises a vent inlet 121-6, which is similar to the vent inlet 121-1. The adhesive patch 106 comprises an adhesive vent aperture 10074. In other examples, an adhesive vent aperture 10074 is not provided. For example, the adhesive patch 106 may extend to cover the vent inlet 121-6. The housing 10002-6 comprises a vent outlet 131-6, which is similar to the vent outlet 131-1, except as described below. The housing 10002-6 comprises a vent passageway 111-6, which is similar to the vent passageway 111-1, except as described below.

Unlike the vent passageway 111-1, the vent passageway 111-6 is connected to the sensor passageway 126. Referring first to the embodiment of FIG. 2F-1, the vent passageway 111-6 comprises a first section 111-6a, a second section 111-6b, and a third section 111-6c. The first section 111-6a extends from the vent inlet 121-6 and connects to the second section 111-6b. The second section 111-6b extends from the first section 111-6a and connects to the third section 111-6c. The third section 111-6c extends from the vent outlet 131-6 and connects to the second section 111-6b. Vent outlet 131-6 also acts as the sharp aperture.

In this embodiment, the first section 111-6a extends in a direction parallel to the central axis A. The second section 111-6b extends in a direction perpendicular to the central axis A. The second section 111-6b extends towards the central axis A. For example, the second section 111-6b extends away from the side surface 10010. In particular, the second section 111-6b extends towards the sensor passageway 126. Thus, the second section 111-6b is perpendicular to the first section 111-6a. In other examples, the angle between the second section 111-6b and the first section 111-6a may not be perpendicular (for example, the angle may be acute or obtuse, such as between 0° and 180°). In this embodiment, the vent passageway 111-6 comprises a corner joining the first section 111-6a to the second section 111-6b. In other examples, the first section 111-6a may be connected to the second section 111-6b by a bend or a curve.

In this embodiment, the third section 111-6c is aligned with the central axis A. Thus, the third section 111-6c is parallel to the first section 111-6a and perpendicular to the second section 111-6b. In other examples, the angle between the second section 111-6b and the third section 111-6c may not be perpendicular (for example, the angle may be acute or obtuse, such as between 0° and 180°). In this embodiment, the vent passageway 111-6 comprises a corner joining the second section 111-6b to the third section 111-6c. In other examples, the third section 111-6c may be connected to the second section 111-6b by a bend or a curve.

The vent outlet 131-6 is aligned with the central axis A. Unlike the housing 10002-1, the housing 10002-6 does not have a separate sharp aperture. Instead, the top surface 10008 has one aperture (the vent outlet 131-6), although other apertures may be provided in other examples. The vent outlet 131-6 performs the function of both the vent outlet 131-1 and the sharp aperture 124. The vent outlet 131-6 is aligned along the central axis A with the sensor aperture 112 in the base surface 10004. The vent outlet 131-6 is configured to receive a sharp for insertion of the analyte sensor. The sharp can therefore pass through the third section 111-6c of the vent passageway 111-6 and then along the sensor passageway 126.

In this manner, the vent outlet 131-6 is connected to the sensor aperture 112. More specifically, the vent passageway 111-6 is connected to the sensor passageway 126. In particular, the third section 111-6c is connected to the sensor passageway 126. The third section 111-6c is aligned with the sensor passageway 126 along the central axis A. In this embodiment, the sensor passageway 126 extends from the sensor aperture 112 to the vent passageway 111-6. The sensor passageway 126 meets the vent passageway 111-6, in this example between the second section 111-6b and the third section 111-6c.

The vent passageway 111-6 is therefore configured to pass water from the vent inlet 121-6 to the vent outlet 131-6 (e.g., along the first section 111-6a, along the second section 111-6b, and along the third section 111-6c). The vent passageway 111-6 may also be configured to pass water from the sensor aperture 112 to the vent outlet 131-6 (e.g., along the sensor passageway 126 and along the third section 111-6c). In this arrangement, a separate vent passageway 111-6 and a separate sensor passageway 126 are not required. Instead, the vent passageway 111-6 is connected to the sensor passageway 126. This can reduce interference with the sensor electronics 122, maximizing room in the housing 10002-6. This arrangement may be particularly useful in cases where the vent inlet 121-6 is located near the sensor aperture 112 since this can reduce the length of the vent passageway 111-6.

In other examples, the second section 111-6b may be omitted, and the first section 111-6a may directly connect the vent inlet 121-6 to the third section 111-6c and thereby connect the vent passageway 111-6 to the sensor passageway 126 (e.g., with an angled passageway).

In some examples, the vent passageway 111-6 may be arranged so that the first section 111-6a and/or the second section 111-6b extends proximally of (e.g., beneath) the sensor electronics 122 (e.g., proximally of a PCB). The second section 111-6b may join the sensor passageway 126 at a position proximally of (e.g., below) the sensor electronics 122. In this manner, the vent passageway 111-6 may not extend through the sensor electronics 122 (except where the already-present sensor passageway 126 is provided), which can increase the space available for the sensor electronics 122.

In this embodiment, the vent passageway 111-6 is positioned off-center in the housing 10002-6. In particular, the vent inlet 121-6 is positioned off-center in the base surface 10004. In other examples, the vent inlet 121-6 may be aligned with the central axis A. For example, the first section 111-6a of the vent passageway 111-6 may be aligned with the central axis A. The vent outlet 131-6 is positioned in the center of the top surface 10008 aligned with the central axis A. In other examples, the vent outlet 131-6 may not be aligned with the central axis A. For example, the vent outlet 131-6 may be positioned off-center in the top surface 111-6. For example, the third section 111-6c of the vent passageway 111-6 may not be aligned with the central axis A. The vent passageway 111-5 may have various positions as explained in relation to the vent passageway 111-1.

In some examples, the sensor passageway 126 may be positioned off-center in the housing 10002-6. For example, the sensor aperture 112 may be positioned off-center in the base surface 10004. The sensor passageway 126 may not be aligned with the central axis A.

In this embodiment, the first section 111-6a of the vent passageway 111-6 is parallel to the central axis A. In other examples, the first section 111-6a may not be parallel to the central axis A. For example, the first section 111-6a may be angled (e.g., see FIGS. 2B-1 and 2B-2). In other examples, the first section 111-6a may be curved or may comprise corners or bends.

In this embodiment, the vent inlet 121-6 and the vent outlet 131-6 are circular, and the first section 111-6a, the second section 111-6b, and the third section 111-6c have a circular cross section. The first section 111-6a has a cylindrical shape having a first width (in this case a diameter). The first width may be equal to the width of the vent inlet 121-6. The third section 111-6c has a cylindrical shape having a third width (in this case a diameter). The third width may be equal to the width of the vent outlet 131-6. In this embodiment, the third width is the same as the first width. In other examples, the widths may be different. The second section 111-6b also has a cylindrical shape having a second width (in this case a diameter). The second width is the same as the first width and the third width. In other examples, the widths may be different.

The vent passageway 111-6 is not limited to having cylindrical-shaped sections. In other examples, the cross-sectional shape may be different. For example, the cross-sectional shape of the first section 111-6a, the second section 111-6b, and/or the third section 111-6c may be a square or other shape. The cross-sectional shape of the first section 111-6a, the second section 111-6b, and the third section 111-6c may be the same or different to each other.

In some examples, the vent passageway 111-6 may comprise at least one further section. For example, the vent passageway 111-6 may comprise three or more sections. In other examples, more than three sections may be provided (e.g., four or more).

In other examples, one or more sections of vent passageway 111-6 may be angled (e.g., see FIGS. 2B-1 and 2B-2). For example, the first section 111-6a may be angled and the second section 111-6b may not be angled (e.g., may be perpendicular to the central axis A). One or more sections of the vent passageway 111-6 may be curved. In the embodiment of FIG. 2F-2, vent passageway 111-6 has section 111-6d the length of which is curved from vent inlet 121-6 to the junction with sensor passageway 126. Stated differently the length of section 111-6d extends along a curved longitudinal axis LA. Vent passageway 111-6 has a second section 111-6e that is straight and aligned with sensor passageway 126 to vent outlet 131-6.

Referring back to FIG. 2F-1, the first section 111-6a, the second section 111-6b, and the third section 111-6c have the same width. In particular, the vent inlet 121-6 has the same size as the vent outlet 131-6. In other examples, the first section 111-6a, the second section 111-6b, and/or the third section 111-6c may have a different width (e.g., diameter). For example, the vent inlet 121-6 may have a different size to the vent outlet 131-6 (e.g., larger or smaller). For example, the first section 111-6a may have a larger width (or smaller) than the second section 111-6b, and/or the second section 111-6b may have a larger width (or smaller) than the third section 111-6c, such as in a stepped configuration (e.g., see FIG. 2D).

In other examples, one or more sections of the vent passageway 111-6 may have a changing width along the length of the section. For example, one or more sections of the vent passageway 111-6 may be tapered (e.g., see FIG. 2C). In other examples, the cross-sectional size of one or more sections of the vent passageway 111-6 changes at a non-constant rate. In cross-section, the passageway wall section may have curved sides. For example, the curvature may increase or decrease along the length of the section of the vent passageway 111-6.

As mentioned, in this embodiment, the vent outlet 131-6 is in the top surface 10008. In other examples, the vent outlet 131-6 is in a different surface of the housing 10002-6. For example, the vent outlet 131-6 may be in the side surface (e.g., see FIGS. 2E-1 and 2E-2). In another example, the vent outlet 131-6 may be in an intermediate surface.

### On-Body Device 100-56

Referring to FIG. 2G, an OBD 100-56 is shown according to an embodiment. OBD 100-56 may be the same as OBD 100-50 of FIGS. 2A-1 to 2A-4, and may include the same features, except as explained below. Various alternative features described in relation to OBD 100-50 may similarly be applied to OBD 100-56 in a corresponding manner. In turn, since OBD 100-50 is similar to OBD 100-1, features of OBD 100-1 may be applied to OBD 100-56. Similarly, the features described in relation to OBDs 100-1′ to 100-32 may be applied to OBD 100-56. Similarly, the features described in relation to OBDs 100-51 to 100-55 may be applied to OBD 100-56.

In this embodiment, OBD 100-56 is the same as OBD 100-50, except that OBD 100-56 has a plurality of vent passageways. In this embodiment, OBD 100-56 comprises a housing 10002-7 with base surface 10004, top surface 10008 and side surface 10010. OBD 100-56 comprises analyte sensor 161. OBD 100-56 comprises an adhesive patch 106. The housing 10002-7 comprises sharp aperture 124, sensor aperture 112, and sensor passageway 126 extending therebetween. In other examples, the housing 10002-7 may have different shapes and may be assembled in different ways, as described herein.

In this embodiment, the base surface 10004 comprises a first vent inlet 121-7(1), which is similar to the vent inlet 121-1. The adhesive patch 106 comprises a first adhesive vent aperture 10074(1) and a second adhesive vent aperture 10074(2). In other examples, adhesive vent apertures 10074(1) and 10074(2) are not provided. For example, the adhesive patch 106 may extend to cover the vent inlet 121-7(1). The housing 10002-7 comprises a vent outlet 131-7(1), which is similar to the vent outlet 131-1. The vent outlet 131-7(1) is in the top surface 10008. The housing 10002-7 comprises a vent passageway 111-7(1), which is similar to the vent passageway 111-1.

Unlike the housing 10002-1, the housing 10002-7 further comprises a second vent passageway 111-7(2). The second vent passageway 111-7(2) may be similar to the first vent passageway 111-7(1). The base surface 10004 comprises a second vent inlet 121-7(2). The top surface 10008 comprises a second vent outlet 131-7(2). The second vent inlet 121-7(2) is connected to the second vent outlet 131-7(2) via the second vent passageway 111-7(2) in a similar manner to the first vent passageway 111-7(1). The first vent passageway 111-7(1) and the second vent passageway 111-7(2) are both similar to the vent passageway 111-1.

In particular, first vent passageway 111-7(1) may be the same as vent passageway 111-1. The second vent passageway 111-7(2) may be the same as the first vent passageway 111-7(1) and thus the same as the vent passageway 111-1, except that the second vent passageway 111-7(2) is positioned in a different location.

In this embodiment, the first vent passageway 111-7(1) and the second vent passageway 111-7(2) are both positioned off-center in the housing 10002-7. In particular, the vent inlets 121-7 are positioned off-center in the base surface 10004 and the vent outlets 131-7 are positioned off-center in the top surface 10008. In other examples, the first vent inlet 121-7(1) or the second vent inlet 121-7(2) may be aligned with the central axis A. The corresponding vent outlet 131-7 may also be aligned with the central axis A. For example, the first vent passageway 111-7(1) or the second vent passageway 111-7(2) may be aligned with the central axis A. Vent passageways 111-7 may have various positions as explained in relation to vent passageway 111-1.

In this embodiment, the first vent passageway 111-7(1) and the second vent passageway 111-7(2) are arranged on different sides of the housing 10002-7 relative to the central axis A (e.g., in different halves of the circular shape of the housing 10002-7). In particular, the central axis A is arranged between the first vent passageway 111-7(1) and the second vent passageway 111-7(2). In other examples, the vent passageways 111-7 may be arranged in different positions, such as on the same side of the housing 10002-7 relative to the central axis A. As mentioned, one of the vent passageways 111-7 may be aligned with the central axis A, and the other vent passageway 111-7 may be off-center.

In this embodiment, the first vent inlet 121-7(1) is arranged to one side of the base surface 10004 and the second vent inlet 121-7(2) is arranged on the opposite side of the base surface 10004 (relative to the central axis A). In other examples, the vent inlets 121-7 may be positioned at other locations, such as on the same side of the base surface 10004 relative to the central axis A. In this embodiment, the first vent outlet 131-7(1) is arranged to one side of the top surface 10008 and the second vent outlet 131-7(2) is arranged on the opposite side of the top surface 10008 (relative to the central axis A). In other examples, the vent outlets 131-7 may be positioned at other locations, such as on the same side of the top surface 10008 relative to the central axis A.

In this embodiment, the vent passageways 111-7 are parallel to the central axis A. In other examples, the first vent passageway 111-7(1) and/or the second vent passageway 111-7(2) may not be parallel to the central axis A. In particular, the vent inlet 121-7 may be offset relative to the corresponding vent outlet 131-7. For example, the first vent passageway 111-7(1) and/or the second vent passageway 111-7(2) may be angled (e.g., see FIGS. 2B-1 and 2B-2). In other examples, first vent passageway 111-7(1) and/or the second vent passageway 111-7(2) may be curved or may comprise corners or bends to allow the vent outlet 131-7 to be positioned at a different location (e.g. offset) to the vent inlet 121-7.

In this embodiment, the width (e.g., diameter) of the vent passageways 111-7 are the same along the length. The width of the vent inlet 121-7 is the same as the width of the vent outlet 131-7. In other examples, the width of the vent passageway 111-7 changes along the length. In other examples, the width of the vent inlet 121-7 is different to the vent outlet 131-7.

In this embodiment, the vent inlets 121-7 and the vent outlets 131-7 are circular, and the first vent passageway 111-7(1) and the second vent passageway 111-7(2) have a circular cross section. The first vent passageway 111-7(1) has a cylindrical shape having a first width (in this case a diameter). The second vent passageway 111-7(2) has a cylindrical shape having a second width (in this case a diameter). The first width may be equal to the second width. In other examples, the widths may be different.

The vent passageways 111-7 are not limited to having cylindrical-shaped sections. In other examples, the cross-sectional shape may be different. For example, the cross-sectional shape of the first vent passageway 111-7(1) and/or the second vent passageway 111-7(2) may be a square or other shape. The cross-sectional shape of the first vent passageway 111-7(1) and/or the second vent passageway 111-7(2) may be the same or different to each other.

In other examples, the housing 10002-7 may comprise at least one further vent passageway 111-7. For example, the housing 10002-7 may comprise three or more vent passageways 111-7. Thus, base surface 10004 may comprise three or more vent inlets 121-7 and top surface 10008 may comprise three or more vent outlets 131-7. In other examples, more than three vent passageways may be provided (e.g., four or more).

In other examples, one or more vent passageways 111-7 may be angled (e.g., see FIGS. 2B-1 and 2B-2). For example, the first vent passageway 111-7(1) may be angled and the second vent passageway 111-7(2) may not be angled (e.g., may be parallel to the central axis A). In other examples, one or more vent passageways 111-7 may be curved.

In other examples, the cross section of one or more of the vent passageways 111-7 may change along the length of the vent passageway 111-7. For example, one or more of the vent passageways 111-7 (e.g., the first vent passageway 111-7(1)) may be tapered (e.g., see FIG. 2C) or have a stepped configuration (e.g., see FIG. 2D). In other examples, the cross-sectional size (e.g., measured by two coordinate axes in a cartesian plane) of one or more of the vent passageways 111-7 changes at a non-constant rate. In cross-section, the passageway wall section may have curved sides. For example, the curvature may increase or decrease along the length of the section of the vent passageway 111-7.

As mentioned, in this embodiment, the vent outlets 131-7 are in the top surface 10008. In other examples, one or more of the vent outlets 131-7 is in a different surface of the housing 10002-7. For example, one or more of the vent outlets 131-7 (e.g., the first vent outlet 131-7(1)) may be in the side surface (e.g., see FIGS. 2E-1 and 2E-2). In another example, the vent outlet 131-7 may be in an intermediate surface.

In other examples, one or more of the vent passageways 111-7 may comprise a plurality of sections, such as a first section and a second section (e.g., see FIGS. 2D, 2E-1, and 2F-1). For example, one or more of the vent passageways 111-7 may comprise a first section and a second section, with the first section having a different width to the second section (e.g., see FIG. 2D). In another example, one or more of the vent passageways 111-7 may comprise a first section and a second section, with the second section connecting the vent passageway 111-7 to the side surface (e.g., see FIG. 2E-1). In another example, one or more of the vent passageways 111-7 may comprise a first section, a second section, and a third section, with the vent passageway 111-7 connecting to the sensor passageway (e.g., see FIG. 2F-1). For example, the first vent passageway 111-7(1) and the second vent passageway 111-7(2) may both connect to the sensor passageway.

In some examples, the first vent passageway 111-7(1) may be connected to the second vent passageway 111-7(2). For example, each vent passageway 111-7 may comprise a first section and a second section, with the second section connecting to a second section of the other vent passageway 111-7. The second sections may connect to a third section shared between the vent passageways 111-7. In this manner, there may be a shared vent outlet 131-7. For example, the first vent inlet 121-7(1) and the second vent inlet 121-7(2) may be connected to the same vent outlet 131-7. In some examples, more than two vent inlets 121-7 may be connected to a single vent outlet 131-7. In some examples, a plurality of vent outlets 131-7 may be connected to a single vent inlet 121-7.

### On-Body Device 100-57

Referring to FIGS. 2H-1 and 2H-2, an OBD 100-57 is shown according to an embodiment. OBD 100-57 may be the same as OBD 100-50 of FIGS. 2A-1 to 2A-4, and may include the same features, except as explained below. Various alternative features described in relation to OBD 100-50 may similarly be applied to OBD 100-57 in a corresponding manner. In turn, since OBD 100-50 is similar to OBD 100-1, features of OBD 100-1 may be applied to OBD 100-57. Similarly, the features described in relation to OBDs 100-1′ to 100-32 may be applied to OBD 100-57. Similarly, the features described in relation to OBDs 100-51 to 100-56 may be applied to OBD 100-57.

In this embodiment, OBD 100-57 is the same as OBD 100-50, except that OBD 100-57 has a plurality of vent passageways in a similar manner to OBD 100-56. OBD 100-57 is the same as OBD 100-13 except that OBD 100-57 comprises a plurality of vent passageways.

OBD 100-57 comprises a housing 10002-8. The housing 10002-8 comprises a first part 17034 and a second part 17036, in a similar manner to OBD 100-8 and OBD 100-13. The first part 17034 includes base surface 10004 (the adhesive patch is removed for illustrative purposes). The base surface 10004 comprises a sensor aperture 112 for receiving the analyte sensor 161A. In this embodiment, reference is made to the analyte sensor 161A, but in other examples, the analyte sensor may be the analyte sensor 161B.

OBD 100-57 comprises sensor electronics 122 including a PCB. Various electronic components are shown such as a battery and an antenna. The electronic components are on a distal side of the PCB. The sensor electronics 122 are assembled onto the first part 17034. The second part 17036 can then be assembled onto the first part 17034, such as described in relation to OBD 100-8 (e.g., see FIG. 1I).

The analyte sensor 161A is shown assembled to the sensor electronics 122 in OBD 100-57 in FIG. 2H-1. In particular, the sensor contacts on the side surface of the first portion 118 are in electrical contact with a connector on the sensor electronics 122. In this manner, the first portion 118 has a side surface in a plane perpendicular to the PCB.

The second part 17036 comprises a top surface 10008. The top surface 10008 comprises a sharp aperture 124 which is in fluid communication with a sensor passageway through OBD 100-57. In this embodiment, the sharp aperture 124 is not aligned with a central axis A, and instead is offset as described herein. In other examples, the sharp aperture 124 may be central. In this embodiment, the housing 10002-8 comprises a side wall 17010. In particular, a first section of the side wall 17010 is on the first part 17034 and a second section is on the second part 17036. The second part 17036 also comprises a curved intermediate surface in a similar manner to OBD 100-7 (e.g., see FIG. 1H). In other examples, the intermediate surface may be angled (e.g., see FIG. 1G), or may be omitted.

The top surface 10008 comprises a plurality of vent outlets 131-8(1), 131-8(2), and 131-8(3). Three vent outlets 131-8 are shown in FIG. 2H-1, but other numbers may be provided in other examples, and/or the vent outlets 131-8 may be positioned in different locations. The vent outlets 131-8 can be smaller than the sharp aperture 124 as shown here.

The base surface 10004 comprises a plurality of vent inlets 121-8(1), 121-8(2), and 121-8(3) corresponding to the vent outlets 131-8(1), 131-8(2), and 131-8(3). The housing 10002-8 comprises a plurality of vent passageways 111-8(1), 111-8(2), and 111-8(3) connected between the vent inlets 121-8 (1), 121-8(2), and 121-8(3) and the vent outlets 131-8(1), 131-8(2), and 131-8(3), respectively. In this embodiment, the second part 17036 comprises portions of the vent passageways 111-8, e.g., passageway walls, extending from an interior surface of the second part 17036, in particular connected to a respective vent outlet 131-8. Each portion of the passageways 111-8(1), 111-8(2), and 111-8(3) extends through a respective aperture 17079(1), 17079(2), and 17079(3) in the PCB of the electronics 122 for connecting to a receptacle (not shown) corresponding to the respective vent inlet 121-8(1), 121-8(2), and 121-8(3). The vent passageways 111-8 are arranged to avoid electronic components on the PCB. In this arrangement, the vent passageways 111-8 may also provide structural support in a similar manner to the supporting posts of OBD 100-14 of FIG. 1N-2. For example, OBD 100-14 may be modified to provide vent passageways through the supporting posts (e.g., by providing vent inlets and vent outlets at either end of the supporting posts). This can provide a dual function and avoids introducing additional apertures through the PCB.

### On-Body Device 100-58

Referring to FIGS. 2I-1 to 2I-5, an OBD 100-58 is shown according to embodiments. OBD 100-58 may be the same as OBD 100-50 of FIGS. 2A-1 to 2A-4, and may include the same features, except as explained below. Various alternative features described in relation to OBD 100-50 may similarly be applied to OBD 100-58 in a corresponding manner. In turn, since OBD 100-50 is similar to OBD 100-1, features of OBD 100-1 may be applied to OBD 100-58. Similarly, the features described in relation to OBDs 100-1′ to 100-32 may be applied to OBD 100-58. Similarly, the features described in relation to OBDs 100-51 to 100-57 may be applied to OBD 100-58.

In this embodiment, OBD 100-58 is the same as OBD 100-50, except that OBD 100-58 has a vent compartment. In this embodiment, OBD 100-58 comprises a housing 10002-9 with base surface 10004, top surface 10008 and side surface 10010. OBD 100-58 comprises analyte sensor 161. OBD 100-58 comprises adhesive patch 106. The housing 10002-9 comprises sharp aperture 124, sensor aperture 112, and sensor passageway 126 extending therebetween. In other examples, the housing 10002-9 may have different shapes and may be assembled in different ways, as described herein.

In this embodiment, the base surface 10004 comprises a vent inlet 121-9, which is similar to the vent inlet 121-1, except as described below. The adhesive patch 106 comprises adhesive vent aperture 10074. In other examples, an adhesive vent aperture 10074 is not provided. For example, the adhesive patch 106 may extend to cover the vent inlet 121-9. The housing 10002-9 comprises a vent outlet 131-9, which is similar to the vent outlet 131-1, except as described below.

Unlike the housing 10002-1, the housing 10002-9 comprises a vent compartment 18080. The vent compartment 18080 is a region between the base surface 10004 and the sensor electronics 122. More specifically, the housing 10002-9 is divided into the vent compartment 18080 and the remaining volume, in which the sensor electronics 122 are located. The sensor electronics 122 are separated from the vent compartment by a dividing surface 18082. In particular, the vent compartment 18080 is sealed from the sensor electronics 122. The dividing surface 18082 extends across the width of the housing 10002-9. In this example, the dividing surface 18082 is similar in size and shape to the base surface 10004.

The vent compartment 18080 is bounded by the base surface 10004 on the proximal side. In other words, the vent compartment 18080 is adjacent to the base surface 10004. The vent compartment 18080 may extend over the entire base surface 10004. The vent compartment 18080 is bounded on the opposite side by the dividing surface 18082 which defines the boundary to the remaining region of the housing 10002-9 containing the sensor electronics 122. The vent compartment 18080 is also bounded by a portion of the side surface 10010. In other words, the vent compartment 18080 is enclosed between the base surface 10004, a part of the side surface 10010, and the dividing surface 18082. In this embodiment, the vent compartment 18080 defines a disc shaped portion of the housing 10002-9.

In this embodiment, the base surface 10004 comprises a plurality of vent inlets 121-9. The adhesive patch 106 comprises a plurality of adhesive vent apertures 10074 corresponding to each vent inlet 121-9. The cross section of FIG. 2I-I shows two such vent inlets 121-9, and a total of four vent inlets 121-9 are present, as shown in FIG. 2I-3. In other examples, there may be other numbers of vent inlets 121-9, such as one, or three or more.

The vent inlets 121-9 are connected to the vent compartment 18080. In this manner, water from the base surface 10004 and/or the adhesive patch 106 can pass through the vent inlets 121-9 into the vent compartment 18080. The vent compartment 18080 can thus replace the vent passageway described herein. For example, the vent compartment may be a form of a vent passageway.

In this embodiment, the side surface 10010 comprises a plurality of vent outlets 131-9. In particular, the vent outlets 131-9 are connected to the part of the side surface 10010 that overlaps with the vent compartment 18080. The cross section of FIG. 2I-I shows two such vent outlets 131-9, and three vent outlets 131-9 are shown in FIG. 2I-2. In other examples, there may be other numbers of vent outlets 131-9, such as one, or three or more.

The vent outlets 131-9 are arranged in the section of the side surface 10010 that defines the periphery of the vent compartment 18080. The vent outlets 131-9 are connected to the vent compartment 18080. In this embodiment, the vent outlets 131-9 are spaced around the circumference of the housing 10002-9.

In this manner, water can pass through the vent inlets 121-9 and into the vent compartment 18080. The water can then pass through the vent outlets 131-9 for release from the housing 10002-9 through the side surface 10010. This arrangement may allow collection of more water due to the volume of the vent compartment 18080. In the embodiments shown in FIGS. 2I-1 through 2I-4, OBD 100-58 can be configured without additional individual vent passageways through the housing 10002-9, which can reduce interference with the sensor electronics 122. The height of the vent compartment 18080 can be minimized to reduce the overall height of the housing 10002-9 and maximize the space for the sensor electronics 122. This can also reduce the path length for water to travel since it can escape through the vent compartment 18080 instead of through the height of the housing 10002-9.

In other examples, the housing 10002-9 may include one or more vent passageways as described herein extending through the housing 10002-9 and connected to the vent compartment 18080. For example, FIG. 2I-5 is a cross-sectional view showing the embodiment of OBD 100-58 of FIG. 2I-1 having vent passageways 111-9(1) and 111-9(2) connected to the vent compartment 18080. Each of the vent passageways 111-9(1) and 111-9(2) have an interior vent inlet 18084(1) and 18084(2), respectively, in dividing surface 18082 at the junction with vent compartment 18080. Each of the vent passageways 111-9(1) and 111-9(2) have a vent outlet 131-11(1) and 131-11(2), respectively, in top surface 10008. The vent passageways 111-9 can pass water vapor from the vent compartment 18080 to the vent outlets 131-11(1) and 131-11(2) and thus provide another route for exiting of water vapor from the body. In this example, the vent compartment 18080 may not include separate vent outlets 131-9 in the side surface 10010. Vent passageways 111-9 can be configured in accordance with any of the vent passageways described herein, including those described with respect to FIGS. 2A-1 through 2H-2.

In other examples, the number and position of the vent inlets 121-9 and the vent outlets 131-9 may be varied. In other examples, the size and shape of the vent inlets 121-9 and the vent outlets 131-9 may be varied, as described herein. For example, the vent outlet 131-9 shape and size can extend down to the adhesive patch 106 as depicted in an alternative embodiment of FIG. 2I-4. In this embodiment vent outlet 131-9 intersects the base surface at its periphery.

### On-Body Device 100-59

Referring to FIGS. 2J-1 and 2J-2, an OBD 100-59 is shown according to an embodiment. OBD 100-59 may be the same as OBD 100-50 of FIGS. 2A-1 to 2A-4, and may include the same features, except as explained below. Various alternative features described in relation to OBD 100-50 may similarly be applied to OBD 100-59 in a corresponding manner. In turn, since OBD 100-50 is similar to OBD 100-1, features of OBD 100-1 may be applied to OBD 100-59. Similarly, the features described in relation to OBDs 100-1′ to 100-32 may be applied to OBD 100-59. Similarly, the features described in relation to OBDs 100-51 to 100-58 may be applied to OBD 100-59.

In this embodiment, OBD 100-59 is the same as OBD 100-50, except that OBD 100-59 has a vent passageway with a different configuration. In this embodiment, OBD 100-59 comprises a housing 10002-10 with base surface 10004, top surface 10008 and side surface 10010. OBD 100-59 comprises analyte sensor 161. OBD 100-59 comprises an adhesive patch 106. In other examples, the housing 10002-10 may have different shapes and may be assembled in different ways, as described herein.

In this embodiment, the base surface 10004 comprises a vent inlet 121-10, which is similar to the vent inlet 121-1, except as described below. In this embodiment, the adhesive patch 106 does not comprise an adhesive vent aperture. In other examples, an adhesive vent aperture may be provided. The housing 10002-10 comprises a vent outlet 131-10, which is similar to the vent outlet 131-1. The housing 10002-10 comprises a vent passageway 111-10, which is similar to the vent passageway 111-1, except as described below.

Unlike the base surface 10004, the base surface 10004 comprises a plurality of channels 3000. The channels 3000 extend along the base surface 10004. The cross section of FIG. 2J-1 shows two channels 3000, and in total eight channels 3000 are present, as shown in FIG. 2J-2. In other examples, there may be other numbers of channels 3000.

The channels 3000 have a depth into the housing 10002-10 (e.g., in a direction parallel to the central axis A) and a length and a width that extends along the base surface 10004. In this embodiment, the depth is equal to the width, and the length is longer than the depth and the width. In other examples, the depth may be different to the width (e.g., larger or smaller).

In this embodiment, the channels 3000 are each connected to the vent inlet 121-10. In particular, a first end of each channel 3000 terminates adjacent to the vent inlet 121-10. The channels 3000 are fluidly connected to the vent inlet 121-10. In some examples, the channels 3000 may extend to vent inlets 121-10 that may in turn be connected to a vent compartment (e.g., vent compartment 18080).

In this embodiment, channels 3000 extend in different directions along the base surface 10004 to form a star shape. Channels 3000 are arranged to extend radially towards the vent inlet 121-10. For example, channels 3000 are spaced at intervals around the vent inlet 121-10. In other examples, other configurations are possible.

A second end of each channel 3000 terminates on the base surface 10004. In other examples, the second end of one or more channels 3000 is arranged at the periphery of the base surface 10004. In other examples, the second end of one or more channels 3000 is connected to the sensor aperture.

In this embodiment, the channels 3000 form recesses or indentations into the base surface 10004. The channels 3000 define a channel opening that is flush with the plane of the base surface 10004. The channels 3000 comprise a distal surface opposite to the channel opening. The distal surface forms a bottom of the channel 3000.

In this manner, water collecting on the base surface 10004, such as from the adhesive patch 106, can be collected by the channels 3000. The channels 3000 provide an increased surface area exposed to the adhesive patch 106, allowing collection of water through the channel openings. The water can then pass along the channels 3000 and to the vent inlet 121-10 for passing through the vent passageway 111-10 to the vent outlet 131-10. In this manner, the channels 3000 can collect moisture from the base surface 10004 and direct it to the vent inlet 121-10. Accordingly, the channels 3000 may be implemented in combination with any of the vent passageways described herein.

The different vent passageways, vent compartments, and accompanying features described with respect to OBDs 100-50 to 20012 of FIGS. 2A to 2K-12 can be freely combined in any combination of two or more in a single OBD, for example, such that any two or more of vent passageways and compartments 111-1, 111-2, 111-3, 111-4, 111-5, 111-6, 111-7, 111-8, 111-9, and 111-10 having different configurations can be included in a single embodiment of the OBD.

FIGS. 2K-1 through 2K-12 show example embodiments of OBDs 100-61 through 100-72, respectively, which can be configured in accordance with any embodiment of OBD 100 described herein. In these embodiments, OBDs 100-61 through 100-72 each has a housing 10002 coupled with adhesive patch 106. OBDs 100-61 through 100-72 in this embodiment are configured for use with a partially implantable sensor (not shown), and each has a sharp aperture 124 leading to a sensor passageway (not shown). In embodiments where OBDs 100-61 through 100-72 are configured for use with a wholly implantable sensor or a wholly ex vivo sensor, sharp aperture 124 (and the sensor passageway) can be omitted or, alternatively, the sharp aperture 124 (and the sensor passageway) can instead be a vent outlet and passageway.

The embodiments of FIGS. 2K-1 through 2K-12 illustrate the many configurations of vent placements that are contemplated herein. All embodiments of OBD 100 described herein can have one vent passageway in fluid communication with a vent inlet and outlet, or two or more vent passageways where each is in fluid communication with a vent inlet and a vent outlet such that vent inlets and outlets are not shared between passageways, or alternatively such that vent inlets and outlets are shared between different passageways. While the number of vent passageways while vary based on the needs of individual discrete OBD designs and applications, all embodiments of OBD 100 described herein can, optionally, have between one and ninety vent passageways. Each of the between one and ninety vent passageways can be in fluid communication with its own (unshared) vent inlet and vent outlet.

In the embodiment of FIG. 2K-1, OBD 100-61 has a housing 10002 has two vent outlets 131-12(1) and 131-12(2) located on opposite sides of top surface 10008, positioned at approximately 180-degree intervals about the center of OBD 100-61 (e.g., in the location of sharp aperture 124). The two vent outlets lead to two vent passageways (not shown) and two vent inlets (not shown), respectively. This configuration is similar to the embodiment shown and described with respect to FIG. 2G, where the vent outlets lead to a vent passageway that extends straight down through OBD 100 along an axis perpendicular (or normal) to the base surface (as shown in FIG. 2G), terminating in a vent inlet on the opposite side of housing that is aligned with the vent outlet. Thus, there is a matching pattern (a mirror image) of vent inlets (and sensor aperture) on the reverse side of housing 10002. Apertures through adhesive patch 106 can optionally be present and aligned with the vent inlets.

FIG. 2K-2 shows an example embodiment of OBD 100-62. Here, three vent outlets 131-12(1), 131-12(2), and 131-12(3) are present in the top surface of the housing. The three vent outlets 131-12 (and corresponding vent passageways and vent inlets) can be positioned in the top surface in any desired location, though more even spacing therebetween can permit more even moisture transport. Here, the three vent outlets are positioned at approximately 120-degree intervals about the center of OBD 100-62 (e.g., in the location of sharp aperture 124).

FIG. 2K-3 shows an example embodiment of OBD 100-63. Here, four vent outlets 131-12 are present in the top surface of the housing. The four vent outlets 131-12 (and corresponding vent passageways and vent inlets) can be positioned in the top surface in any desired location, though more even spacing therebetween can permit more even moisture transport. Here, the four vent outlets 131-12 are positioned at approximately 90-degree intervals about the center of OBD 100-63 (e.g., the location of sharp aperture 124).

FIG. 2K-4 shows an example embodiment of OBD 100-64. Here, five vent outlets 131-12 are present in the top surface of the housing. The five vent outlets 131-12 (and corresponding vent passageways and vent inlets) can be positioned in the top surface in any desired location, though more even spacing therebetween can permit more even moisture transport. Here, the five vent outlets 131-12 are positioned at approximately 72-degree intervals about the center of OBD 100-64 (e.g., the location of sharp aperture 124).

FIG. 2K-5 shows an example embodiment of OBD 100-65. Here, six vent outlets 131-12 are present in the top surface of the housing. The six vent outlets 131-12 (and corresponding vent passageways and vent inlets) can be positioned in the top surface in any desired location, though more even spacing therebetween can permit more even moisture transport. Here, the six vent outlets 131-12 are positioned at approximately 60-degree intervals about the center of OBD 100-65 (e.g., the location of sharp aperture 124).

FIG. 2K-6 shows an example embodiment of OBD 100-66. Here, seven vent outlets 131-12 are present in the top surface of the housing. The seven vent outlets 131-12 (and corresponding vent passageways and vent inlets) can be positioned in the top surface in any desired location, though more even spacing therebetween can permit more even moisture transport. Here, the seven vent outlets 131-12 are positioned at approximately 51-degree intervals about the center of OBD 100-66 (e.g., the location of sharp aperture 124).

FIG. 2K-7 shows an example embodiment of OBD 100-67. Here, eight vent outlets 131-12 are present in the top surface of the housing. The eight vent outlets 131-12 (and corresponding vent passageways and vent inlets) can be positioned in the top surface in any desired location, though more even spacing therebetween can permit more even moisture transport. Here, the eight vent outlets 131-12 are positioned at approximately 45-degree intervals about the center of OBD 100-67 (e.g., the location of sharp aperture 124).

FIG. 2K-8 shows an example embodiment of OBD 100-68 that illustrates how one or more vent outlets (and corresponding vent passageways and vent inlets) can be located at different radial spacings between the center and periphery of OBD 100 in the embodiments herein. Here, eight vent outlets 131-12(1) through 131-12(8) are present in the top surface of the housing. Like FIG. 2K-7, the eight vent outlets are positioned at approximately 45-degree intervals about the center of OBD 100-68 (e.g., in the location of sharp aperture 124). However, in this embodiment the spacing of the vent outlets 131-12 from the center (and the periphery) of OBD 100-68 is not the same. Vent outlets 131-12(1) through 131-12(4) are each located at approximately the same distance from the center (and the periphery) at a first distance. Vent outlets 131-12(5) through 131-12(8) are each located at approximately the same distance from the center (and the periphery) at a second distance that is different than the first distance. Vent outlets 131-12(1) through 131-12(4) are located relatively farther from the center than vent outlets 131-12(5) through 131-12(8).

FIG. 2K-9 shows an example embodiment of OBD 100-69 that illustrates how one or more vent outlets (and corresponding vent passageways and vent inlets) can be located at different locations and combined with a vent compartment in OBD 100 in the embodiments herein. Here, three vent outlets 131-12 are present in the top surface of the housing and three vent outlets 131-14(1), 131-14(2), and 131-14(3) are present in the side surface 10010 of housing 10002 (locations of outlets 131-14(2) and 131-14(3) are indicated with arrows as they are obscured). Vent outlets 131-12 lead to a vent passageway and vent inlet (not shown) and vent outlets 131-14 lead to a vent compartment (not shown), such that this embodiment has a configuration with both vent passageways and a vent compartment like that of FIG. 2I-5. Vent outlets 131-12 and corresponding vent passageways and vent inlets can be omitted such that the embodiment is like that of FIG. 2I-1 through 2I-4. Like FIG. 2K-2, the three vent outlets 131-12 are positioned at approximately 120-degree intervals about the center of OBD 100-69 (e.g., in the location of sharp aperture 124). The three vent outlets 131-14(1), 131-14(2), and 131-14(3) are also positioned at approximately 120-degree intervals about the center of OBD 100-69 but offset such that each outlet 131-14 is between two top surface outlets 131-12.

FIG. 2K-10 shows an example embodiment of OBD 100-70 similar to the embodiment of FIG. 2K-9 but with four vent outlets 131 present in the top surface of the housing and four vent outlets 131-14(1), 131-14(2), 131-14(3) and 131-14(4) are present in the side surface 10010 of housing 10002 (locations of outlets 131-14(3) and 131-14(4) are indicated with arrows as they are obscured). Like FIG. 2K-3, the four vent outlets 131 are positioned at approximately 90-degree intervals about the center of OBD 100-70 (e.g., in the location of sharp aperture 124). The four vent outlets 131-14(1), 131-14(2), 131-14(3) and 131-14(4) are also positioned at approximately 90-degree intervals about the center of OBD 100-70 but offset such that each outlet 131-14 is between two top surface outlets 131.

FIG. 2K-11 shows an example embodiment of OBD 100-71 similar to the embodiment of FIG. 2K-9 but showing that there can be more side surface vent outlets 131-14 than top surface vent outlets 131. Here, four vent outlets 131 are present in the top surface of the housing and eight vent outlets 131-14 (indicated by arrows) are present in the side surface of housing 10002. Like FIG. 2K-3, the four vent outlets 131 are positioned at approximately 90-degree intervals about the center of OBD 100-71 (e.g., in the location of sharp aperture 124). The eight vent outlets 131-14 are positioned at approximately 45-degree intervals about the center of OBD 100-71, with four outlets 131-14 aligned with outlets 131 (0, 90, 180, and 270 degs.) and four outlets 131-14 at positions between outlets 131.

FIG. 2K-12 shows an example embodiment of OBD 100-72 similar to the embodiment of FIG. 2K-9 but showing that there can be less side surface vent outlets 131-14 than top surface vent outlets 131. Here, eight vent outlets 131 are present in the top surface of the housing and four vent outlets 131-14 (indicated by arrows) are present in the side surface of housing 10002. Like FIG. 2K-8, the eight vent outlets 131 are positioned at approximately 45-degree intervals about the center of OBD 100-72 (e.g., in the location of sharp aperture 124). The four vent outlets 131-14 are positioned at approximately 90-degree intervals about the center of OBD 100-72 at positions between outlets 131 (45, 135, 225, and 315 degs.).

The vent outlets 131, vent inlets, and vent passageways can be configured in accordance with any of the embodiments described herein, including all of those shown and described with respect to FIGS. 2A-1 through 2H-2 and 2I-5. The vent outlets 131, though shown on the top surface of the housing, can be alternatively (or additionally) implemented on the side surface to implement the vent configurations of the embodiments of FIGS. 2E-1 and 2E-2. Likewise, the vent outlets 131 can be consolidated in one or more outlets on the top surface to implement the vent configurations of the embodiments of FIGS. 2F-1 and 2F-2.

### Vent Seal

The following section describes embodiments of seals for vent passageways and sensor passageways, including all of the vent and sensor passageways described herein with respect to the embodiments of OBDs 100-50 to 100-72 of FIGS. 2A-1 to 2K-12. This includes a description of moisture permeable seals in various configurations and shapes such as layers, liners, cylinders, bulbs or bulbous forms, rivets, plugs, caps, and stickers. According to the various embodiments, these moisture permeable seals can be added to OBD 100 during assembly, after assembly and prior to deployment on the body of the wearer, or after deployment on the body of the wearer. Any two or more of the seals described with respect to FIGS. 4A-1 through 6B-3 can be combined and used in for the same or different passageways in a single embodiment of OBD 100.

The seals in these embodiments are moisture permeable (MP). The MP seal acts as an impediment to the free movement of liquid into the vent and/or sensor passageway while allowing moisture vapor to exit. The MP seal can impede movement of liquid through itself (and the vent and/or sensor passageway) freely and at scale, such as may be the case if the wearer were bathing or showering and subjecting the OBD to immersion or a direct stream of liquid water. The MP seal can prevent the movement of liquid through itself altogether during the operable lifetime of OBD 100. The MP seal allows accumulated moisture to exit the region between the OBD housing and the wearer's body by evaporation into a vapor and then migration (e.g., diffusion, and/or venting) through the MP seal into the ambient external environment away from the wearer's body. Examples of such accumulated moisture can be sweat, bodily fluid from the insertion site in the wearer's body, condensed atmospheric moisture, and/or moisture from the external environment that may have seeped into the region between the housing and the wearer's body (for example during activities with a high propensity for fluid exposure such as swimming, bathing and showering).

One manner in which the MP seal has this capability is by being composed of at least one moisture permeable material. A moisture permeable material allows moisture vapor migration while impeding liquid on the basis of its molecular chemistry and/or microscopic structure. The MP seal can be a composite and/or have a combined construction of two or more moisture permeable materials, or a moisture permeable material with a non-moisture permeable material. The moisture permeable material can have a nonwoven or woven form.

Silicone, which is a non-organic elastomeric polymer also referred to as polysiloxane, is an example of a moisture permeable material suitable for use with the embodiments described herein. The moisture permeable material can be one of the following types of silicone, though not limited to such: silicone rubber; room temperature vulcanizing silicone (RTV-1, RTV-2 silicone); or fluorosilicone. A moisture permeable fibrous material such as synthetic flashspun high-density polyethylene fibers (e.g., TYVEK) is another example suitable for use with the embodiments described herein. Poly(methyl methacrylate) (PMMA) (e.g., acrylic, PLEXIGLAS) is another example of a moisture permeable material suitable for use with the embodiments described herein unless otherwise noted. Additional examples of moisture permeable materials usable in the embodiments described herein include but are not limited to: polyurethane (PU); polytetrafluoroethylene (PTFE); polyethylene (PE); polypropylene (PP); and nylon (polyamide). A micro-porous polymer composite can be used as the MP material. Such a composition can include polyolefins like PE or PP blended with an inorganic filler, e.g., calcium carbonate, along with film stretching to helps create porous voids in the matrix. The aforementioned moisture permeable materials can form the entire seal structure itself or can be in the form of a film or coating over another liquid permeable porous material that forms the majority of the seal structure. The moisture permeable material can be hygroscopic. The moisture permeable material can be hydrophilic or can have a hydrophilic coating.

The MP seal can have its moisture permeability varied by its dimensions. Generally, the moisture permeability of the seal will be inversely proportional to the distance through the material which moisture must migrate to pass entirely (e.g., the thickness of the seal). Thus, an MP seal that is thinner will generally be more moisture permeable than a thicker one of the same composition. The embodiments of the MP seals described herein placed within passageways can have a thickness that extends the length of the passageway (vent or sensor), but generally have a thickness that is less than 50% of the length of passageway. The thickness of the MP seals in passageways (or portions of MP seals in passageways) can be thinner, such as from 1%-35% of the length of the passageway, and/or can be still thinner, such as from 1%-25% of the length of the passageway. Other thicknesses can be utilized as described in other embodiments herein, including those that are greater than 50% of the length of the passageway.

The MP seal can have its moisture permeability increased by placement of small perforations in the seal. In the embodiments herein, a seal with perforations can become moisture permeable even if constructed from a non-moisture permeable material, such as a hydrophobic material or material bearing a hydrophobic coating. In some embodiments, the perforations are of a width that impedes the free movement of liquid while allowing gas migration, such as diameters between 10 and 1000 microns, between 100 and 700 microns, between 200 and 600 microns, and/or between 300 and 500 microns. The perforations can be placed in the material by the use of cutting (e.g., laser) or can be formed at the same time the seal is formed by way of a patterned mold, to name several examples.

Moisture permeability of the MP seal and OBD 100 having the MP seal can be quantified according to the Moisture Vapor Transmission Rate (MVTR). MVTR can be assessed at or near body temperature (37.8 degrees Celsius) according to American Society for Testing and Materials (ASTM) F1249. In the embodiments herein, the MP seal and OBD 100 can each have an MVTR of 35 grams per square meter per hour (g/m2/hour) or more for benefits pertaining to regulation of skin surface pH, skin irritation, and maceration when the vent aperture is in close contact with the skin, although the embodiments are not limited to such. The MP seal and OBD 100 can each have an MVTR of at least 35 g/m2/hr up to 500 g/m2/hr in many embodiments, though again not limited to such.

As used herein, the term “MP seal 222” refers collectively to all embodiments of MP seals described herein, including MP seals 222-1 through 222-65. FIG. 3A-1 is a cross-sectional view of OBD 100-50 of FIG. 2A-1 except having MP seals 222 according to an embodiment. Specifically, in this embodiment OBD 100-50 has a first MP seal 222-1 located in a top or upper region of vent passageway 111-1 adjacent outlet 131-1 and top surface 10008. First MP seal 222-1 has a seal top surface 2008-1 that is beneath top surface 10008, but in other embodiments surface 2008-1 is flush with top surface 10008 (see, e.g., FIGS. 5A-3, 5D-2) or above top surface 10008. MP seal 222-1 has a seal bottom surface 2004-1 that is also in the top or upper region of vent passageway 111-1, such that the entirety of MP seal 222-1 resides in the upper region of vent passageway 111-1. The thickness of first MP seal 222-1 is less than 50% of the length of vent passageway 111-1. The thickness of first MP seal 222-1 can be less than 35% of the length of vent passageway 111-1. The thickness of first MP seal 222-1 can be still thinner, such as less than 25% of the length of vent passageway 111-1, as shown here. Other thicknesses can be utilized as described in other embodiments herein, including those that are greater than 50% of the length of the passageway.

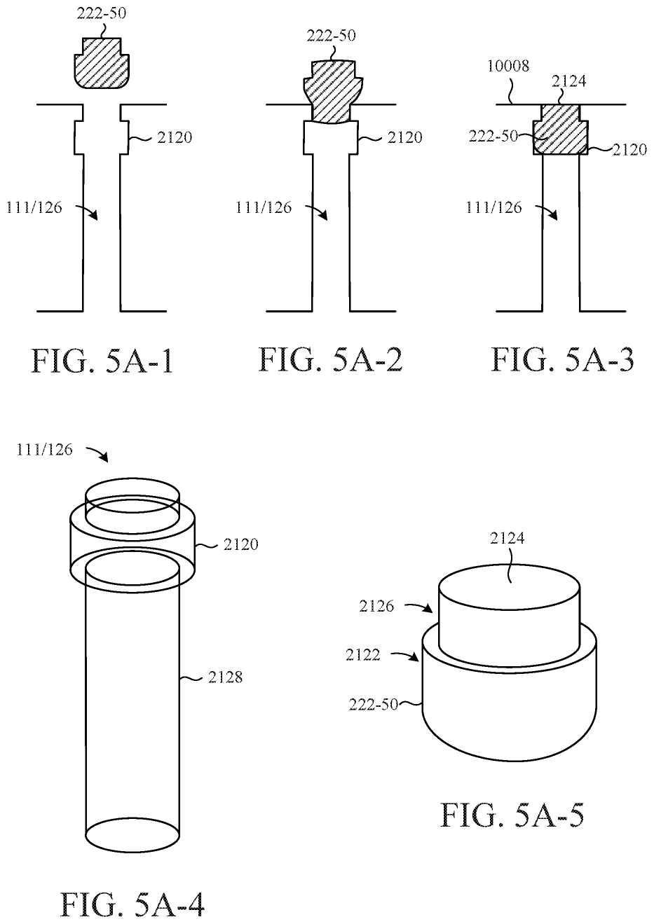

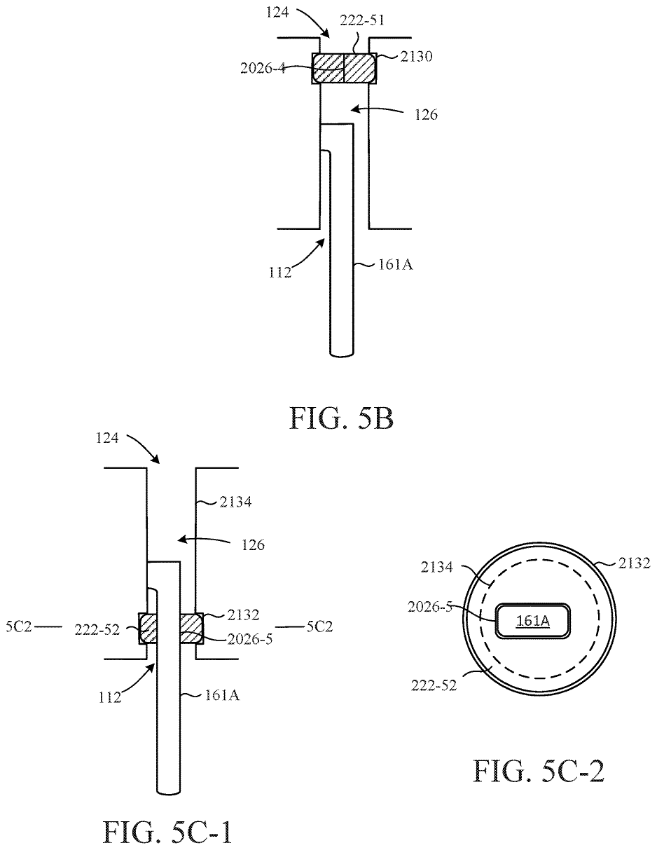

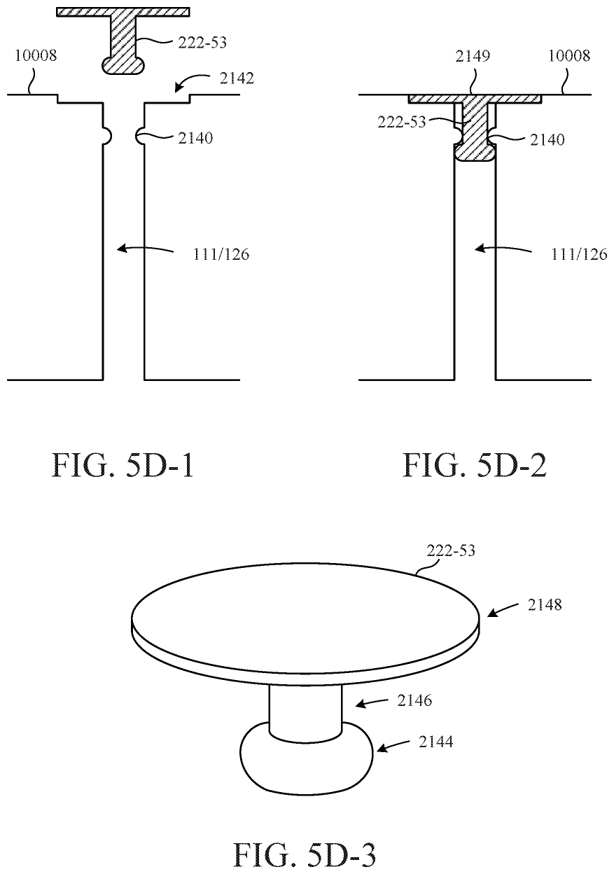

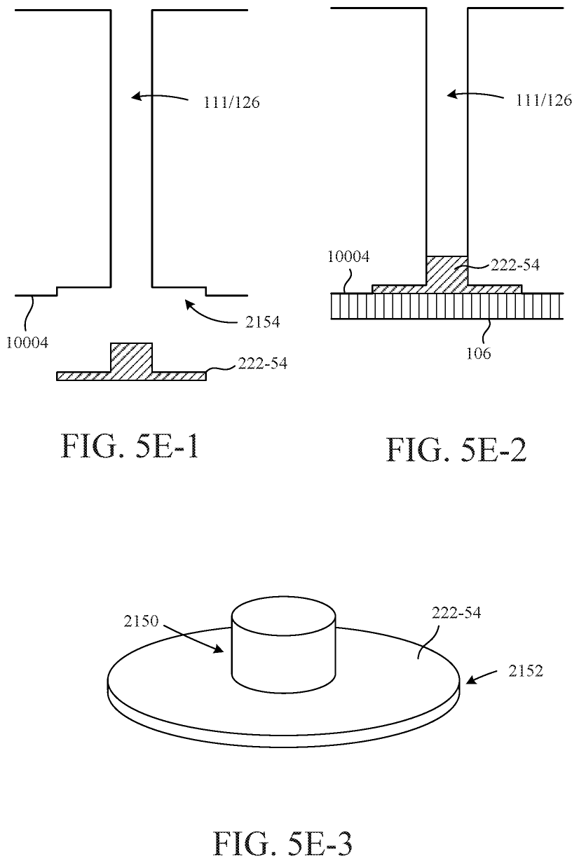

OBD 100-50 also has a second MP seal 222-2 located in a bottom or lower region of vent passageway 111-1 adjacent inlet 121-1 and base surface 10004. Second MP seal 222-2 has a seal bottom surface 2004-2 that is above base surface 10008, but in other embodiments surface 2004-2 is flush with base surface 10004 (see, e.g., FIG. 5E-2) or below base surface 10004. MP seal 222-2 has a seal top surface 2008-2 that is also in the bottom or lower region of vent passageway 111-1, such that the entirety of MP seal 222-2 resides in the lower region of vent passageway 111-1. The thickness of second MP seal 222-2 is less than 50% of the length of vent passageway 111-1. The thickness of second MP seal 222-2 can be less than 35% of the length of vent passageway 111-1. The thickness of second MP seal 222-2 can be still thinner, such as less than 25% of the length of vent passageway 111-1. Other thicknesses can be utilized as described in other embodiments herein, including those that are greater than 50%.

OBD 100-50 has a third MP seal 222-3 located in a top or upper region of sensor passageway 126 adjacent top surface 10008. Third MP seal 222-3 has a seal top surface 2008-3 that is beneath top surface 10008, but in other embodiments surface 2008-3 is flush with top surface 10008 (see, e.g., FIG. 5A-3, 5D-2, 6B-3) or above top surface 10008. MP seal 222-3 has a seal bottom surface 2004-3 that is also in the top or upper region of sensor passageway 126, such that the entirety of third MP seal 222-3 resides in the upper region of sensor passageway 126. The thickness of third MP seal 222-3 is less than 50% of the length of sensor passageway 126. The thickness of third MP seal 222-3 can be less than 35% of the length of sensor passageway 126. The thickness of third MP seal 222-3 can be still thinner, such as less than 25% of the length of sensor passageway 126. Other thicknesses can be utilized as described in other embodiments herein, including those that are greater than 50%.

OBD 100-50 also has a fourth MP seal 222-4 located in a bottom or lower region of sensor passageway 126 adjacent base surface 10004. Fourth MP seal 222-4 has a seal bottom surface 2004-4 that is above base surface 10008, but in other embodiments surface 2004-4 is flush with base surface 10004 (see, e.g., FIG. 5E-2) or below base surface 10004. MP seal 222-4 has a seal top surface 2008-4 that is also in the bottom or lower region of sensor passageway 126, such that the entirety of fourth MP seal 222-4 resides in the lower region of sensor passageway 126. The thickness of MP seal 222-4 is less than 50% of the length of sensor passageway 126. The thickness of MP seal 222-4 can be less than 35% of the length of sensor passageway 126. The thickness of MP seal 222-4 can be still thinner, such as less than 25% of the length of sensor passageway 126. Other thicknesses can be utilized as described in other embodiments herein, including those that are greater than 50%.

Third MP seal 222-3 has an inner passageway 2026-1 that permits movement of the sharp of an inserter through MP seal 222-3. Third MP seal 222-3 can comprise an elastic MP material that is biased to close inner passageway 2026-1 after removal of the sharp. Fourth MP seal 222-4 has an inner passageway 2026-2 that permits movement of the sharp of an inserter through MP seal 222-4. Inner passageway 2026-2 also permits presence of sensor 161 as shown here. Fourth MP seal 222-4 can comprise an elastic MP material that is biased to close inner passageway 2026-2 after removal of the sharp, which in this embodiment closes and presses inner passageway 2026-2 against the surfaces of sensor 161. This has the effect of sealing around sensor 161 and also stabilizing sensor 161 against movement, such as movement of the body relative to the OBD and sensor 161, and movement of the sensor 161 itself, such as vibrations during shipping or transport.

Inner passageways 2026-1 and 2026-2 can be formed in the respective MP seals 222-3 and 222-4 prior to insertion of the sharp, such as by cutting (e.g., laser), use of a mold having a post or member to form the separation during the molding process, or insertion of a piercing member different than the sharp. Inner passageways 2026-1 and 2026-2 can be formed in the respective MP seals 222-3 and 222-4 by insertion of the sharp itself, such as during assembly of the sharp and OBD during manufacturing and/or prior to sterilization.

In the embodiment of FIG. 3A-1, vent passageway 111-1 has two MP seals 222-1 and 222-2 and sensor passageway 126 has two MP seals 222-3 and 222-4. Different implementations of OBD 100 may have different requirements for sealing and thus may utilize more or less than two MP seals per passageway. In an alternative embodiment, vent passageway 111-1 only has first (upper) MP seal 222-1 and second (lower) MP seal 222-2 is omitted. In another alternative embodiment, vent passageway 111-1 only has second (lower) MP seal 222-2 and first (upper) MP seal 222-1 is omitted. In yet another embodiment, sensor passageway 126 only has MP seal 222-3 (upper) and MP seal 222-4 (lower) is omitted. In still another embodiment, sensor passageway 126 only has MP seal 222-4 (lower) and MP seal 222-3 (upper) is omitted. These embodiments can be combined as desired in an OBD, for example, such that vent passageway 111-1 has one MP seal and sensor passageway 126 has one MP seal, such that vent passageway 111-1 has one MP seal and sensor passageway 126 has two MP seals, or such that vent passageway 111-1 has two MP seals and sensor passageway 126 has one MP seal, where those seals are in any location within the respective passageways.

FIG. 3A-2 is a cross-sectional view of OBD 100-50 of FIG. 2A-1 except having MP seals according to an embodiment. Specifically, in this embodiment OBD 100-50 has a first MP seal 222-5 located in an intermediate region of vent passageway 111-1 between base surface 10004 and top surface 10008. First MP seal 222-5 has a seal top surface 2008-5 that is beneath top surface 10008 and a seal bottom surface 2004-5 that is above base surface 10004, such that the entirety of MP seal 222-5 resides in the intermediate region of vent passageway 111-1. First MP seal 222-5 can be located in the center of the length of vent passageway 111-1 as shown here or can be offset from center. The thickness of MP seal 222-5 is less than 50% of the length of vent passageway 111-1. The thickness of MP seal 222-5 can be less than 35% of the length of vent passageway 111-1. The thickness of MP seal 222-5 can be still thinner, such as less than 25% of the length of vent passageway 111-1. Other thicknesses can be utilized as described in other embodiments herein, including those that are greater than 50%.

OBD 100-50 also has a second MP seal 222-6 located in an intermediate region of sensor passageway 126 between top surface 10008 and base surface 10004. Second MP seal 222-6 has a seal top surface 2008-6 that is beneath top surface 10008 and a seal bottom surface 2004-6 that is above base surface 10004, such that the entirety of MP seal 222-6 resides in the intermediate region of sensor passageway 126. Second MP seal 222-6 can be located in the center of the length of sensor passageway 126 as shown here or can be offset from center. The thickness of MP seal 222-6 is less than 50% of the length of sensor passageway 126. The thickness of MP seal 222-6 can be less than 35% of the length of sensor passageway 126. The thickness of MP seal 222-6 can be still thinner, such as less than 25% of the length of sensor passageway 126. Other thicknesses can be utilized as described in other embodiments herein, including those that are greater than 50%. Second MP seal 222-6 has an inner passageway 2026-3 that permits movement of the sharp of an inserter through MP seal 222-6. Inner passageway 2026-3 also permits presence of sensor 10016 as shown here.

The embodiment of FIG. 3A-2 having MP seals 222-5 and 222-6 in intermediate locations within vent passageway 111-1 and sensor passageway 126, respectively, can be combined with other embodiments to have more than one MP seal in each passageway. For example, vent passageway 111-1 can have intermediate MP seal 222-5 along with one or both of MP seals 222-1 and/or 222-2 shown in FIG. 3A-1. Similarly, sensor passageway 126 can have intermediate MP seal 222-6 along with one or both of MP seals 222-3 and/or 222-4 shown in FIG. 3A-1. These various configurations for MP seals within vent passageway 111-1 can be combined with any of these various configurations for MP seals within sensor passageway 126.

FIG. 3B-1 is a cross-sectional view of OBD 100-51 of FIG. 2B-1 except having MP seals according to an embodiment. Specifically, OBD 100-51 has first (upper) MP seal 222-7 and second (lower) MP seal 222-8 in vent passageway 111-2. MP seals 222-7 and 222-8 have generally oblique cylindrical shapes and are otherwise similar to MP seals 222-1 and 222-2, respectively, of the embodiment of FIG. 3A-1. MP seals 222-7 and 222-8 can additionally or alternatively be configured in accordance with any of the variations described for MP seals 222-1 and 222-2, respectively, including omission of either MP seal 222-7 or 222-8. In this embodiment OBD 100-51 has third MP seal 222-3 and fourth MP seal 222-4 in sensor passageway 126. MP seals 222-3 and 222-4 can be configured and/or omitted in accordance with any of the variations described with respect to FIG. 3A-1.

FIG. 3B-2 is a cross-sectional view of OBD 100-51 of FIG. 2B-1 except having MP seals according to an embodiment. Specifically, OBD 100-51 has first (intermediate) MP seal 222-9, with a generally oblique cylinder shape, in vent passageway 111-2 and second (intermediate) MP seal 222-6 in sensor passageway 126. MP seals 222-9 is similar to MP seal 222-5 of the embodiment of FIG. 3A-2. MP seal 222-9 can additionally or alternatively be configured in accordance with any of the variations described for MP seal 222-5. MP seal 222-6 can be configured and/or omitted in accordance with any of the variations described with respect to FIG. 3A-1.

FIG. 3B-3 is a cross-sectional view of OBD 100-51 of FIG. 2B-2 except having MP seals according to an embodiment. Specifically, OBD 100-51 has first (upper) MP seal 222-10 and second (lower) MP seal 222-11 in vent passageway 111-2. MP seals 222-10 and 222-11 have generally oblique cylinder shapes and are otherwise similar to MP seals 222-1 and 222-2, respectively, of the embodiment of FIG. 3A-1. MP seals 222-10 and 222-11 can additionally or alternatively be configured in accordance with any of the variations described for MP seals 222-1 and 222-2, respectively, including omission of either MP seal 222-10 or 222-11. In this embodiment OBD 100-51 has third MP seal 222-3 and fourth MP seal 222-4 in sensor passageway 126. MP seals 222-3 and 222-4 can be configured and/or omitted in accordance with any of the variations described with respect to FIG. 3A-1.

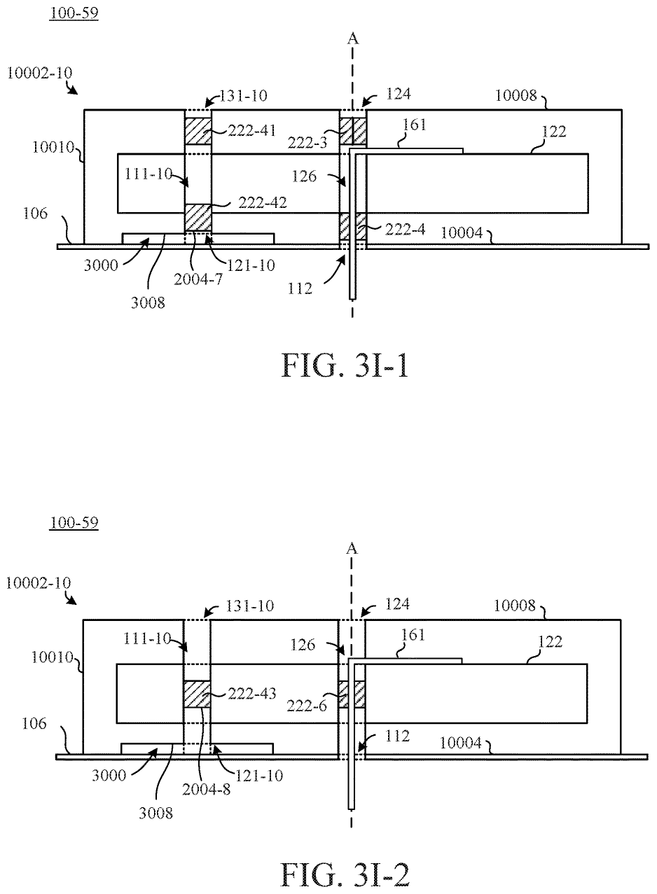

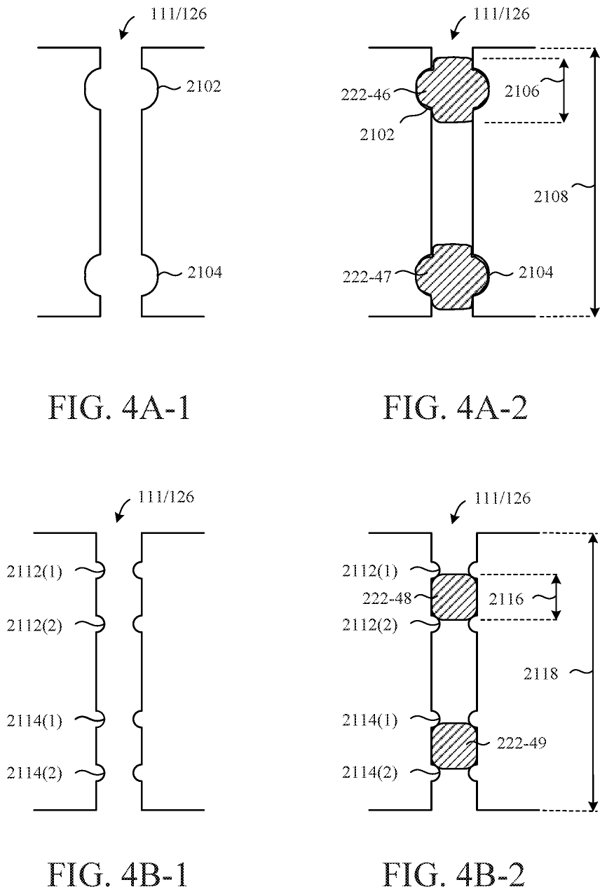

FIG. 3B-4 is a cross-sectional view of OBD 100-51 of FIG. 2B-2 except having MP seals according to an embodiment. Specifically, OBD 100-51 has first (intermediate) MP seal 222-12 in vent passageway 111-2 and second (intermediate) MP seal 222-6 in sensor passageway 126. MP seal 222-12 has a generally oblique cylinder shape. MP seal 222-12 is otherwise similar to MP seal 222-5 of the embodiment of FIG. 3A-2. MP seal 222-12 can additionally or alternatively be configured in accordance with any of the variations described for MP seal 222-5. MP seal 222-6 can be configured and/or omitted in accordance with any of the variations described with respect to FIG. 3A-1. The embodiments herein include MP seals of FIGS. 3B-1, 3B-2, 3B-3 and 3B-4 that are configured to fit within (e.g., match) all the variations in passageway shape described with respect to FIGS. 2B-1 and 2B-2.

Although in the embodiments of FIGS. 3B-1, 3B-2, 3B-3, and 3B-4 the sensor passageway 126 is depicted as a generally non-oblique cylinder shape, the sensor passageway can also be configured as an oblique cylinder similar to the vent passageways 111-2 of these embodiments.

FIG. 3C-1 is a cross-sectional view of OBD 100-52 of FIG. 2C except having MP seals according to an embodiment. Specifically, OBD 100-52 has first (upper) MP seal 222-14 and second (lower) MP seal 222-15 in vent passageway 111-3. MP seals 222-14 and 222-15 have generally tapered cylinder shapes (e.g., frusto-conical) and are otherwise similar to MP seals 222-1 and 222-2, respectively, of the embodiment of FIG. 3A-1. MP seals 222-14 and 222-15 can additionally or alternatively be configured in accordance with any of the variations described for MP seals 222-1 and 222-2, respectively, including omission of either MP seal 222-14 or 222-15. In this embodiment OBD 100-52 has third MP seal 222-3 and fourth MP seal 222-4 in sensor passageway 126. MP seals 222-3 and 222-4 can be configured and/or omitted in accordance with any of the variations described with respect to FIG. 3A-1.

FIG. 3C-2 is a cross-sectional view of OBD 100-52 of FIG. 2C except having MP seals according to an embodiment. Specifically, OBD 100-52 has first (intermediate) MP seal 222-16, with a generally tapered cylinder shape, in vent passageway 111-3 and second (intermediate) MP seal 222-6 in sensor passageway 126. MP seal 222-16 is similar to MP seal 222-5 of the embodiment of FIG. 3A-2. MP seal 222-16 can additionally or alternatively be configured in accordance with any of the variations described for MP seal 222-5. MP seal 222-6 can be configured and/or omitted in accordance with any of the variations described with respect to FIG. 3A-1.

FIG. 3C-3 is a cross-sectional view of the embodiment of FIG. 3C-1 except with a reversal of the orientation of the taper for vent passageway 111-3 such that the narrower end is at vent inlet 121-3 and the wider end of passageway 111-3 is at vent outlet 131-3. Specifically, OBD 100-52 has first (upper) MP seal 222-17 and second (lower) MP seal 222-18 in vent passageway 111-3. MP seals 222-17 and 222-18 have generally tapered cylinder shapes and are otherwise similar to MP seals 222-1 and 222-2, respectively, of the embodiment of FIG. 3A-1. MP seals 222-17 and 222-18 can additionally or alternatively be configured in accordance with any of the variations described for MP seals 222-1 and 222-2, respectively, including omission of either MP seal 222-17 or 222-18. In this embodiment OBD 100-52 has third MP seal 222-3 and fourth MP seal 222-4 in sensor passageway 126. MP seals 222-3 and 222-4 are similar to MP seals 222-3 and 222-4, respectively, of the embodiment of FIG. 3A-1. MP seals 222-3 and 222-4 can additionally or alternatively be configured in accordance with any of the variations described for MP seals 222-3 and 222-4, respectively.

FIG. 3C-4 is a cross-sectional view of the embodiment of FIG. 3C-2 except, as in FIG. 3C-3, with a reversal of the orientation of the taper for vent passageway 111-3 such that the narrower end is at vent inlet 121-3 and the wider end of passageway 111-3 is at vent outlet 131-3. Specifically, OBD 100-52 has first (intermediate) MP seal 222-19, with a generally tapered cylinder shape, in vent passageway 111-3 and second (intermediate) MP seal 222-6 in sensor passageway 126. MP seal 222-19 is similar to MP seal 222-5 of the embodiment of FIG. 3A-2. MP seal 222-19 can additionally or alternatively be configured in accordance with any of the variations described for MP seal 222-5. MP seal 222-6 can be configured and/or omitted in accordance with any of the variations described with respect to FIG. 3A-1.

The tapered configuration of the vent passageway 111-3 of the embodiments of FIGS. 3C-1, 3C-2, 3C-3, and 3C-4 can be beneficial in construction of OBD 100-52. For example, if the MP seal is formed by injection of liquid MP material into passageway 111-3 (as described herein), the tapered configuration can act to trap the liquid MP material in place within the narrowing channel. The embodiments herein include MP seals of FIGS. 3C-1, 3C-2, 3C-3, and 3C-4 that are configured to fit within (e.g., match) all the variations in passageway shape described with respect to FIGS. 2C-1 and 2C-2.

Although in the embodiments of FIGS. 3C-1, 3C-2, 3C-3, and 3C-4 the sensor passageway 126 is depicted as a generally non-oblique cylinder shape, the sensor passageway can also have a tapered configuration similar to the vent passageways 111-3 of these embodiments.

FIG. 3D-1 is a cross-sectional view of OBD 100-53 of FIG. 2D except having MP seals according to an embodiment. Specifically, OBD 100-53 has first (upper) MP seal 222-20 and second (lower) MP seal 222-21 in vent passageway 111-4. MP seal 222-20 has a relatively smaller width (or diameter) than MP seal 222-21. MP seals 222-20 and 222-21 are otherwise similar to MP seals 222-1 and 222-2, respectively, of the embodiment of FIG. 3A-1. MP seals 222-20 and 222-21 can additionally or alternatively be configured in accordance with any of the variations described for MP seals 222-1 and 222-2, respectively, including omission of either MP seal 222-20 or 222-21. In this embodiment OBD 100-53 has third MP seal 222-3 and fourth MP seal 222-4 in sensor passageway 126. MP seals 222-3 and 222-4 can be configured and/or omitted in accordance with any of the variations described with respect to FIG. 3A-1.

FIG. 3D-2 is a cross-sectional view of OBD 100-53 of FIG. 2D except having MP seals according to an embodiment. Specifically, OBD 100-53 has first (intermediate) MP seal 222-22 in vent passageway 111-4 and second (intermediate) MP seal 222-6 in sensor passageway 126. MP seal 222-22 has a relatively smaller width (or diameter) than MP seal 222-6. MP seal 222-22 is otherwise similar to MP seal 222-5 of the embodiment of FIG. 3A-2. MP seal 222-22 can additionally or alternatively be configured in accordance with any of the variations described for MP seal 222-5. MP seal 222-6 can be configured and/or omitted in accordance with any of the variations described with respect to FIG. 3A-1. The embodiments herein include MP seals of FIGS. 3D-1 and 3D-2 that are configured to fit within (e.g., match) all the variations in passageway shape described with respect to FIGS. 2D-1 and 2D-2.

Although in the embodiments of FIGS. 3D-1 and 3D-2 the sensor passageway 126 is depicted as a generally non-oblique cylinder shape, the sensor passageway can also have a varying width configuration similar to the vent passageways 111-4 of these embodiments.

FIG. 3E-1 is a cross-sectional view of OBD 100-54 of FIG. 2E-1 except having MP seals according to an embodiment. Specifically, OBD 100-54 has first (upper) MP seal 222-23 and second (lower) MP seal 222-24 in vent passageway 111-5. Specifically, MP seal 222-23 is located within second section 111-5b of vent passageway 111-5 flush against side surface 10010 of housing 10002-5. MP seal 222-24 is located within first section 111-5a. MP seals 222-23 and 222-24 are similar to MP seals 222-1 and 222-2, respectively, of the embodiment of FIG. 3A-1. MP seals 222-23 and 222-24 can additionally or alternatively be configured in accordance with any of the variations described for MP seals 222-1 and 222-2, respectively, including omission of either MP seal 222-23 or 222-24. In this embodiment OBD 100-54 has third MP seal 222-3 and fourth MP seal 222-4 in sensor passageway 126. MP seals 222-3 and 222-4 can be configured and/or omitted in accordance with any of the variations described with respect to FIG. 3A-1.

FIG. 3E-2 is a cross-sectional view of OBD 100-54 of FIG. 2E-1 except having MP seals according to an embodiment. Specifically, OBD 100-54 has first (intermediate) MP seal 222-25 in vent passageway 111-5 and second (intermediate) MP seal 222-6 in sensor passageway 126. MP seal 222-25 is similar to MP seal 222-5 of the embodiment of FIG. 3A-2. MP seal 222-25 can additionally or alternatively be configured in accordance with any of the variations described for MP seal 222-5. MP seal 222-6 can be configured and/or omitted in accordance with any of the variations described with respect to FIG. 3A-1. The embodiments herein include MP seals of FIGS. 3E-1 and 3E-2 that are configured to fit within (e.g., match) all the variations in passageway shape described with respect to FIG. 2E.

Although in the embodiments of FIGS. 3E-1 and 3E-2 the sensor passageway 126 is depicted as a generally non-oblique cylinder shape, the sensor passageway 126 can also have a bent or elbow configuration similar to the vent passageways 111-5 of these embodiments.

FIG. 3E-3 is a cross-sectional view of OBD 100-54 of FIG. 2E-1 except having MP seals according to an embodiment. Specifically, OBD 100-54 has first (upper) MP seal 222-26 and second (lower) MP seal 222-27 in vent passageway 111-5. Specifically, MP seal 222-26 is located within vent passageway 111-5 flush against side surface 10010 of housing 10002-5. MP seals 222-26 and 222-27 have curved shapes that correspond to the shape of curved vent passageway 111-5 and are otherwise similar to MP seals 222-1 and 222-2, respectively, of the embodiment of FIG. 3A-1. MP seals 222-26 and 222-27 can additionally or alternatively be configured in accordance with any of the variations described for MP seals 222-1 and 222-2, respectively, including omission of either MP seal 222-26 or 222-27. In this embodiment OBD 100-54 has third MP seal 222-3 and fourth MP seal 222-4 in sensor passageway 126. MP seals 222-3 and 222-4 can be configured and/or omitted in accordance with any of the variations described with respect to FIG. 3A-1.

FIG. 3E-4 is a cross-sectional view of OBD 100-54 of FIG. 2E-1 except having MP seals according to an embodiment. Specifically, OBD 100-54 has first (intermediate) MP seal 222-28 in vent passageway 111-5 and second (intermediate) MP seal 222-6 in sensor passageway 126. MP seal 222-28 has a curved shape that correspond to the shape of curved vent passageway 111-5. MP seal 222-28 is otherwise similar to MP seal 222-5 of the embodiment of FIG. 3A-2. MP seal 222-28 can additionally or alternatively be configured in accordance with any of the variations described for MP seal 222-5. MP seal 222-6 can be configured and/or omitted in accordance with any of the variations described with respect to FIG. 3A-1. The embodiments herein include MP seals of FIGS. 3E-3 and 3E-4 that are configured to fit within (e.g., match) all the variations in passageway shape described with respect to FIGS. 2E-1 and 2E-2.

Although in the embodiments of FIGS. 3E-1 and 3E-2 the sensor passageway 126 is depicted as a generally non-oblique cylinder shape, the sensor passageway 126 can also have a bent or elbow configuration similar to the vent passageways 111-5 of these embodiments.

FIG. 3F-1 is a cross-sectional view of OBD 100-55 of FIG. 2F-1 except having MP seals according to an embodiment. Specifically, OBD 100-55 has first (upper) MP seal 222-29 in second section 111-6b of vent passageway 111-6 and second (lower) MP seal 222-30 in first section 111-6a of vent passageway 111-6. MP seals 222-29 and 222-30 are otherwise similar to MP seals 222-1 and 222-2, respectively, of the embodiment of FIG. 3A-1. MP seals 222-29 and 222-30 can additionally or alternatively be configured in accordance with any of the variations described for MP seals 222-1 and 222-2, respectively, including omission of either MP seal 222-29 or 222-30. In this embodiment OBD 100-55 has third MP seal 222-3 and fourth MP seal 222-4 in sensor passageway 126. MP seals 222-3 and 222-4 can be configured and/or omitted in accordance with any of the variations described with respect to FIG. 3A-1. If MP seal 222-29 in this embodiment is omitted at least one seal is still provided per opening in housing (MP seal 222-30 for vent inlet 121-6, MP seal 222-3 for vent outlet 131-6, and MP seal 222-4 for sensor aperture 112).

FIG. 3F-2 is a cross-sectional view of OBD 100-55 of FIG. 2F-1 except having an MP seal according to an embodiment. Specifically, OBD 100-55 has an MP seal 222-31 at the junction between the second section 111-6b and the third section 111-6c of vent passageway 111-6. MP Seal 222-31 extends along and across both second section 111-6b and third section 111-6c such that the single MP seal 222-31 provides the moisture permeable sealing function for both vent passageway 111-6 and sensor passageway 126. Approximately half of MP seal 222-31 is in each of the passageway sections. MP seal 222-31 is otherwise similar to MP seal 222-5 of the embodiment of FIG. 3A-2. MP seal 222-31 can additionally or alternatively be configured in accordance with any of the variations described for MP seal 222-5. The embodiments herein include MP seals of FIGS. 3F-1 and 3F-2 that are configured to fit within (e.g., match) all the variations in passageway shape described with respect to FIG. 2F-1.

FIG. 3F-3 is a cross-sectional view of OBD 100-55 of FIG. 2F except having MP seals according to an embodiment. Specifically, OBD 100-55 has first (upper) MP seal 222-32 in vent passageway section 111-6d and second (lower) MP seal 222-33 in vent passageway section 111-6d. MP seals 222-32 and 222-33 have curved shapes that correspond to the shape of curved vent passageway section 111-6d and are otherwise similar to MP seals 222-1 and 222-2, respectively, of the embodiment of FIG. 3A-1. MP seals 222-32 and 222-33 can additionally or alternatively be configured in accordance with any of the variations described for MP seals 222-1 and 222-2, respectively, including omission of either MP seal 222-32 or 222-33. In this embodiment OBD 100-55 has third MP seal 222-3 and fourth MP seal 222-4 in sensor passageway 126. MP seals 222-3 and 222-4 can be configured and/or omitted in accordance with any of the variations described with respect to FIG. 3A-1. If MP seal 222-32 in this embodiment is omitted at least one seal is still provided per opening in housing (MP seal 222-33 for vent inlet 121-6, MP seal 222-3 for vent outlet 131-6, and MP seal 222-4 for sensor aperture 112).

FIG. 3F-4 is a cross-sectional view of OBD 100-55 of FIG. 2F except having an MP seal according to an embodiment. Specifically, OBD 100-55 has an MP seal 222-34 at the junction between vent passageway section 111-6d and section 111-6e. MP Seal 222-34 extends along and across both section 111-6d and section 111-6e (as well as sharp passageway 15026) such that the single MP seal 222-34 provides the moisture permeable sealing function for both vent passageway 111-6 and sensor passageway 126. Approximately half of MP seal 222-34 is in each of the passageway sections. MP seal 222-34 is otherwise similar to MP seal 222-5 of the embodiment of FIG. 3A-2. MP seal 222-34 can additionally or alternatively be configured in accordance with any of the variations described for MP seal 222-5. The embodiments herein include MP seals of FIGS. 3F-1 and 3F-2 that are configured to fit within (e.g., match) all the variations in passageway shape described with respect to FIG. 2F-1 and 2F-2.

FIG. 3G-1 is a cross-sectional view of OBD 100-56 of FIG. 2G except having MP seals according to an embodiment. Specifically, OBD 100-56 has first (upper) MP seal 222-36(1) and second (lower) MP seal 222-37(1) in vent passageway 111-7(1) and first (upper) MP seal 222-36(2) and second (lower) MP seal 222-37(2) in vent passageway 111-7(2). MP seals 222-36(1) and 222-36(2), are similar to MP seal 222-1 of the embodiment of FIG. 3A-1, and MP seals 222-37(3) and 222-37(4) are similar to MP seal 222-2 of the same figure. MP seals 222-36(1) and 222-36(2) can additionally or alternatively be configured in accordance with any of the variations described for MP seal 222-1, including omission of either or both of MP seals 222-36(1) and 222-36(2). MP seals 222-37(1) and 222-37(2) can additionally or alternatively be configured in accordance with any of the variations described for MP seal 222-2, including omission of either or both of MP seals 222-37(1) and 222-37(2). In this embodiment OBD 100-56 has third MP seal 222-3 and fourth MP seal 222-4 in sensor passageway 126. MP seals 222-3 and 222-4 can be configured and/or omitted in accordance with any of the variations described with respect to FIG. 3A-1.

FIG. 3G-2 is a cross-sectional view of OBD 100-56 of FIG. 2G except having MP seals according to an embodiment. Specifically, OBD 100-56 has first (intermediate) MP seal 222-38(1) in vent passageway 111-7(1), second (intermediate) MP seal 222-38(2) in vent passageway 111-7(2), and third (intermediate) MP seal 222-6 in sensor passageway 126. MP seals 222-38(1) and 222-38(2) are similar to MP seal 222-5 of the embodiment of FIG. 3A-2. MP seals 222-38(1) and 222-38(2) can additionally or alternatively be configured in accordance with any of the variations described for MP seal 222-5. MP seal 222-6 can be configured and/or omitted in accordance with any of the variations described with respect to FIG. 3A-1.

FIG. 3H-1 is a cross-sectional view of OBD 100-58 of FIG. 2I-1 except having MP seals according to an embodiment. Specifically, OBD 100-58 has an MP seal 222-65 along a portion of the inner surface of vent compartment 18080. MP seal 222-65 is configured as a liner or layer that covers vent outlets 131-9 and vent inlets 121-9 along the bottom inner surface and the side inner surface of the vent compartment 18080 as shown. In alternative embodiments the MP seal 222-65 can cover only the vent outlets 131-9 (e.g., by positioning of the MP seal along the side surface of the vent compartment and not the bottom surface) or only the vent inlets 121-9 (e.g., by positioning the MP seal along the bottom surface of the vent compartment and not the side surface). In another embodiment MP seal 222-65 is placed along the exterior surface of housing 10002-9 over vent outlets 131-9 (e.g., along the exterior side surface of the housing) and/or vent inlets 121-9 (e.g., between base surface 10004 and patch 106). OBD 100-58 also has third MP seal 222-3 and fourth MP seal 222-4 in sensor passageway 126. MP seals 222-3 and 222-4 can be configured and/or omitted in accordance with any of the variations described with respect to FIG. 3A-1.

FIG. 3H-2 is a cross-sectional view of OBD 100-58 similar to that of FIGS. 3G-1, 3H-1, and FIG. 2I-5 except having MP seals in vent passageways 111-9 according to an embodiment. Specifically, OBD 100-58 has first (upper) MP seal 222-39(1) and second (lower) MP seal 222-40(1) in vent passageway 111-9(1) and first (upper) MP seal 222-39(2) and second (lower) MP seal 222-40(2) in vent passageway 111-9(2). MP seals 222-39(1) and 222-39(2), are similar to MP seal 222-1 of the embodiment of FIG. 3A-1, and MP seals 222-40(1) and 222-40(2) are similar to MP seal 222-2 of the same figure. MP seals 222-39(1) and 222-39(2) can additionally or alternatively be configured in accordance with any of the variations described for MP seal 222-1, including omission of either or both of MP seals 222-39(1) and 222-39(2). MP seals 222-40(1) and 222-40(2) can additionally or alternatively be configured in accordance with any of the variations described for MP seal 222-2, including omission of either or both of MP seals 222-40(1) and 222-40(2). In this embodiment OBD 100-58 has third MP seal 222-3 and fourth MP seal 222-4 in sensor passageway 126. MP seals 222-3 and 222-4 can be configured and/or omitted in accordance with any of the variations described with respect to FIG. 3A-1. In this embodiment, two MP seals are present in each passageway 111-9 in upper and lower locations, but one MP seal can be present in an intermediate location similar to the embodiment of FIG. 3G-2.

FIG. 3I-1 is a cross-sectional view of OBD 100-59 of FIG. 2J-1 except having MP seals according to an embodiment. Specifically, OBD 100-59 has first (upper) MP seal 222-41 and second (lower) MP seal 222-42 in vent passageway 111-10. MP seals 222-41 and 222-42 are similar to MP seals 222-1 and 222-2, respectively, of the embodiment of FIG. 3A-1, except the lower surface 2004-7 of MP seal 222-42 is above the uppermost surface 3008 of channel 3000 as shown here. Alternatively surface 2004-7 can be flush with the uppermost surface 3008 of channel 3000. MP seals 222-41 and 222-42 can additionally or alternatively be configured in accordance with any of the variations described for MP seals 222-1 and 222-2, respectively, including omission of either of MP seals 222-41 and 222-42. In this embodiment OBD 100-59 has third MP seal 222-3 and fourth MP seal 222-4 in sensor passageway 126. MP seals 222-3 and 222-4 can be configured and/or omitted in accordance with any of the variations described with respect to FIG. 3A-1.

FIG. 3I-2 is a cross-sectional view of OBD 100-59 of FIG. 2J-1 except having MP seals according to an embodiment. Specifically, OBD 100-59 has first (intermediate) MP seal 222-43 in vent passageway 111-10 and second (intermediate) MP seal 222-6 in sensor passageway 126. MP seal 222-43 is similar to MP seal 222-5 of the embodiment of FIG. 3A-2, with the lower surface 2004-8 of MP seal 222-42 above the uppermost surface 3008 of channel 3000. MP seal 222-43 can additionally or alternatively be configured in accordance with any of the variations described for MP seal 222-5. MP seal 222-6 can be configured and/or omitted in accordance with any of the variations described with respect to FIG. 3A-1.

Although in the embodiments of FIGS. 3I-1 and 3I-2 the sensor passageway 126 is depicted as a not connected to a channel 3000, the sensor passageway 126 can also be connected to the same or a different channel 3000 like with the vent passageways 111-10 of these embodiments.

The shapes and sizes of the MP seals shown in FIGS. 3A-1 through 3G-2 and FIGS. 3I-1 and 3I-2 can vary based on the seal's manner of construction, the shape of the interior location of the passageway where the MP seal is placed, and the desired thickness for moisture permeability purposes. The embodiments of the MP seals shown and described with respect to these figures can have, but are not limited to, cylindrical shapes (generally or otherwise), oblique cylindrical shapes (generally or otherwise), tapered cylindrical shapes (generally or otherwise), bulb or bulbous shapes, plug shapes, layer or layer-like shapes, button shapes, and the like.

### Seal Integration Through Injection

Each of the MP seals described with respect to FIGS. 3A-1 to 31-2 can have various different shapes, manners of fabrication, and manners of placement within or on OBD 100. For example, the MP seals depicted in FIGS. 3A-1 to 3G-2 and 3I-1 to 31-2 can be placed within vent and/or sensor passageways by injection of the MP material (e.g., silicone) directly into the desired location within the passageway. The MP material adheres to the wall of the passageway and becomes fixed in position within the passageway as it cures. One or more features, such as a groove, ridge, slot, projection, or recess, can be formed by the wall of the passageways to assist in securing the injected MP material and prevent dislodgement during and after curing. Seals placed through injection can have shapes that conform to the adjacent surfaces. A generally cylindrical passageway can result in an MP seal shape that is also generally cylindrical. Passageways of different shapes will generally result in similar shapes for the MP seals.

FIG. 4A-1 is a cross-sectional view depicting a passageway 111/126, which can be a vent passageway 111 or a sensor passageway 126 (sensor not shown) of any of the embodiments herein, having an upper annular recess 2102 and a lower annular recess 2104 prior to seal formation. FIG. 4A-2 is a cross-sectional view depicting passageway 111/126 after seal formation, with a first MP seal 222-46 formed by injection of MP material within passageway 111/126, with the MP material filling both the passageway 111/126 and recess 2102 at the upper location. Second MP seal 222-47 is formed within passageway 111/126 by injection of MP material therein, with the MP material filling both the passageway 111/126 and lower recess 2104 at the lower location. As the MP material cools and cures, the MP material of first MP seal 222-46 adheres to the walls of the passageway 111/126 and forms a shape that interlocks with recess 2102. Similarly, the MP material of second MP seal 222-47 adheres to the walls of the passageway 111/126 and forms a shape that interlocks with recess 2104. In this manner removal or dislodgement of MP seals 222-46 and 222-47 is inhibited. In this embodiment, the thickness 2106 of MP seal 222-46 is approximately 25% of the length 2108 of passageway 111/126. In this example, MP seals 222-46 and 222-47 have bulbous shapes.

Recesses 2102 and 2104 can extend around the entire circumference of passageway 111/126 in an annular form or can be non-annular and extend around only a part or portion of the entire circumference of passageway 111/126. In this embodiment recesses 2102 and 2104 are used to facilitate creation of MP seals at the upper and lower locations within passageway 111/126, and in other embodiments one or more recesses can be used to facilitate creation of one or more MP seals at any location within passageway 111/126, such as a lower location, intermediate location, and/or an upper location.

FIG. 4B-1 is a cross-sectional view depicting a passageway 111/126, which can be a vent passageway 111 or a sensor passageway 126 (sensor not shown) of any of the embodiments herein, having a pair of annular projections 2112(1) and 2112(2) at an upper location and a pair of annular projections 2114(1) and 2114(2) at a lower location within the passageway 111/126. prior to seal formation. FIG. 4B-2 is a cross-sectional view depicting passageway 111/126 after seal formation, with a first MP seal 222-48 formed by injection of MP material within passageway 111/126, with the MP material filling the passageway 111/126 between annular projections 2112(1) and 2112(2) at the upper location. Second MP seal 222-49 is formed within passageway 111/126 by injection of MP material therein, with the MP material filling the passageway 111/126 between annular projections 2114(1) and 2114(2) at the lower location. As the MP material cools and cures, the MP material of first MP seal 222-48 adheres to the walls of the passageway 111/126 and forms a shape that is buttressed by projections 2112(1) and 2112(2). Similarly, the MP material of second MP seal 222-49 adheres to the walls of the passageway 111/126 and forms a shape that is buttressed by projections 2114(1) and 2114(2). In this manner removal or dislodgement of MP seals 222-48 and 222-49 is inhibited. In this embodiment, the thickness 2116 of MP seal 222-46 is less than 20% of the length 2118 of passageway 111/126. In this example, MP seals 222-48 and 222-49 have generally cylindrical, or bulbous, shapes.

Projections 2112 and 2114 can extend around the entire circumference of passageway 111/126 in their annular form or can be non-annular and extend around only a part or portion of the entire circumference of passageway 111/126. Also, based on the orientation of OBD during the curing process, one of projections 2112(1) and 2112(2) can be omitted if the remaining projections buttresses the MP seal while curing, and similarly one of projections 2114(1) and 2114(2) can be omitted. With a moisture permeable material having a relatively fast curing rate, for example, both pairs of projections 2112 and 2114 can be omitted and the material can be injected directly into the passageway with no special feature for retention of the material in a specific location. In this embodiment projections 2112 and 2114 are used to facilitate creation of MP seals at the upper and lower locations within passageway 111/126, and in other embodiments one or more projections can be used to facilitate creation of one or more MP seals at any location within passageway 111/126, such as a lower location, intermediate location, and/or an upper location.

As described herein, the housing of OBD 100 can be formed from two or more pieces or parts that are connected together in an assembly process (see, e.g., FIG. 1I, 2H-1, and 2H-2). In a two-part housing, at least a portion of the vent passageway can be formed in or otherwise integral to one part, and the remaining portion of the vent passageway can be formed in or otherwise integral to the other part. The portions of the vent passageway can be connected when the housing is assembled. If the two parts are subjected to different sterilization processes due to, e.g., the presence of the sensor in one part, it can be advantageous to locate the moisture permeable seal in the part that does not have the sensor so that it is not subject to as high of a sterilization requirement. This may alleviate a constraint on the practical implementation of the moisture permeable seal. Thus, in an example embodiment, a method of assembling an OBD can include, placing a moisture permeable seal in a portion of a vent passageway in a first part of an OBD housing and subjecting the first part to a first sterilization process. The method can further include subjecting a second part of the housing to a second sterilization process different from the first sterilization process. The second sterilization process can require a condition that may degrade or alter the integrity of the moisture permeable material (e.g., electron beam sterilization). The method can then include assembling the first part with the second part. The second part can include a sensor, such as a partially in vivo sensor, during the sterilization process. The moisture permeable seal can be placed in a location within the vent passageway that corresponds to an intermediate or upper location as described in the embodiments herein (e.g., FIG. 3A-1 and 3A-2). The seal can be placed in the vent passageway by liquid injection or by insertion of a preformed seal during assembly (e.g., see discussion below).

### Seal Integration Through Assembly

As stated, each of the MP seals described with respect to FIGS. 3A-1 to 31-2 can have various different shapes, sizes, manners of fabrication, and manners of placement within or on OBD 100. As an alternative to injection, in some embodiments the MP seals depicted in FIGS. 3A-1 to 3G-2 and 3I-1 to 3I-2 can be integrated into OBD 100 by an assembly process that includes inserting, pushing, and/or pressing the MP seal into the passageway (vent or sharp). The MP seal can be held in place within the passageway by an interference fit. In some embodiments the MP seal is formed of an elastic material that is biased towards a state with a shape suitable for sealing the passageway and is deformed from that state during the insertion process. The MP seal then returns to or near to the original state within the passageway, in a position that interlocks the MP seal with a feature within the passageway configured to hold the seal and resist dislodgement. Embodiments of such deformable MP seals are described with respect to FIGS. 5A-1 to 5D-3.

FIGS. 5A-1, 5A-2, and 5A-3 are cross-sectional views depicting states of an MP seal assembly process, where an MP seal 222-50 in inserted into a passageway 111/126, which can be a vent passageway 111 or sensor passageway 126 of any of the embodiments herein. Passageway 111/126 has a recess 2120 as shown in the perspective view of FIG. 5A-4, which depicts surfaces of the sidewall 2128 and recess 2120 of passageway 111/126 within an OBD. FIG. 5A-1 depicts MP seal 222-50 in its at-rest state prior to insertion. FIG. 5A-2 depicts MP seal 222-50 deformed from the at-rest state to a deformed state during insertion into passageway 111/126. FIG. 5A-3 depicts MP seal 222-50 fully inserted into passageway 111/126 and partially residing within the recess 2120 in passageway 111/126. Here, MP seal 222-50 returns to its at-rest state, or a state near to the at-rest state if the size of MP seal 222-50 is slightly larger than the corresponding space within recess 2120 and passageway 111/126. MP seal 222-50 is shown here in the upper portion of the passageway 111/126, but the configuration of seal 222-50, the location of recess 2120, and the assembly process can be similarly utilized to place an MP seal in an intermediate position or lower position within passageway.

FIG. 5A-5 is a perspective view depicting MP seal 222-50 in more detail, having a flange portion 2122 with an upper columnar portion 2126. The width of flange portion 2122 is greater than the width of columnar portion 2126 such that flange portion 2122 is configured to reside in recess 2120 with columnar portion 2126 residing in passageway 111/126. The height of columnar portion 2126 is such that an upper surface 2124 of columnar portion 2122 is flush with a top surface 10008 of the OBD housing (see FIG. 5A-3). In this example, MP seal 222-50 has a plug shape, or bulbous shape. A similar configuration can be used for an MP seal placed in a lower position within a passageway, where the columnar portion projects downward such that its lower surface is flush with a base surface of the OBD housing. In alternative embodiments, the MP seal can have a columnar portion projecting from both the upper and lower sides of the flange portion.

FIG. 5B is a cross-sectional view depicting an embodiment of a MP seal 222-51 placed within a recess 2130 within the upper portion of a sensor passageway 126. Here, MP seal 222-51 has an inner passageway 2026-4 for allowing movement of a sharp therethrough. Sensor 21214 is shown below MP seal 222-51. In this example, MP seal 222-51 has a generally cylindrical, or button shape.

FIG. 5C-1 is a cross-sectional view depicting an embodiment of an MP seal 222-52 placed within a recess 2132 within the lower portion of a sensor passageway 126 having sidewall 2134. Here, MP seal 222-52 has an inner passageway 2026-5 for allowing movement of a sharp therethrough. Sensor 161 is shown within inner passageway 2026-5. FIG. 5C-2 is a cross-sectional view taken along line 5C2-5C2 of FIG. 5C-1 and depicts a top-down view of MP seal 222-52 within recess 2132, with sensor 161A within inner passageway 2026-5. MP seal 222-52 has transitioned toward the at-rest state where inner passageway is pressed into contact with the circumference of sensor 161A to effectuate the seal. In this example, MP seal 222-52 has a generally cylindrical, or button shape. Similar configurations can be used instead with sensor 161B.

FIGS. 5D-1 and 5D-2 are cross-sectional views depicting an embodiment of an MP seal 222-53 before and after insertion into a passageway 111/126, which can be a vent passageway 111 or sensor passageway 126 of any of the embodiments herein. FIG. 5D-3 is a perspective view depicting MP seal 222-53 in more detail, having a flange portion 2144 connected with a columnar portion 2146 in turn connected to a head portion 2148. The width of flange portion 2144 is greater than the width of columnar portion 2146 such that flange portion 2144, which is configured to deform during insertion, can slide past annular projection 2140 and resist dislodgement. Head portion 2148 is wider than passageway 111/126 and resides within an inset 2142 against top surface 10008 of the OBD and, in cooperation with flange portion 2144, secures MP seal in position. A top surface 2149 of head portion 2148 can be flush with the adjacent uppermost part of surface 10008 of the OBD housing as shown here. In this example, MP seal 222-53 has a plug or rivet shape. A similar configuration can be used for an MP seal placed in a lower position within a passageway.

FIG. 5E-1 is a cross-sectional view depicting an embodiment of an MP seal 222-54 within a passageway 111/126, which can be a vent or sensor passageway of any of the embodiments herein. FIG. 5E-2 is a perspective view depicting MP seal 222-54 in more detail, having a columnar portion 2150 connected to a head portion 2152. The width of head portion 2152 is greater than the width of columnar portion 2150 and passageway 111/126. In this embodiment columnar portion 2150 can have a width generally the same as the width of passageway 111/126, or slightly greater. Head portion 2152 resides within an inset 2154 against base surface 10004 of the OBD. Adhesive patch 106 is positioned against and adhesively coupled with base surface 10004 and holds MP seal 222-54 in place within passageway 111/126. Thus, in this configuration no further mechanism (such as a recess or projection within passageway that interfaces with an oversized portion of the MP seal) is required to secure MP seal 222-54 in place within passageway 111/126. In this example, MP seal 222-54 has a plug or cap shape.

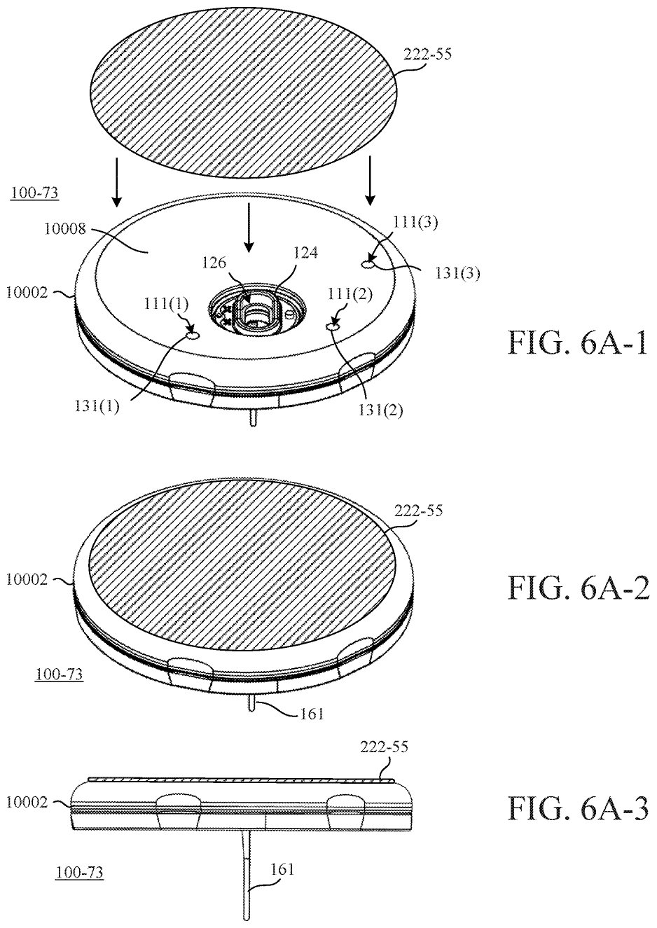

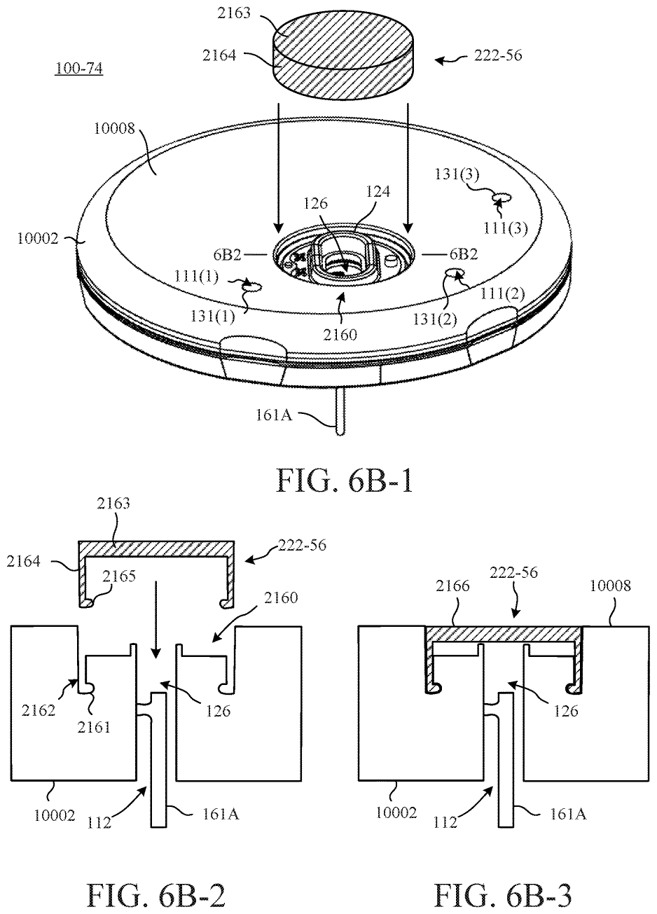

FIGS. 6A-1 and 6A-2 are perspective views depicting an example embodiment of an OBD 100-73 similar to that of FIGS. 2H-1 and 2H-2. In this embodiment OBD 100-60 has a housing 10002 with three vent outlets 131(1), 131(2), and 131(3) in fluid communication with vent passageways 111(1), 111(2), and 111(3) and corresponding vent inlets (obscured). OBD housing 10002 also as a sharp aperture 124 in fluid communication with a sensor passageway 126 and a sensor aperture (obscured) through which sensor 161 extends. FIG. 6A-1 also depicts an MP seal 222-55, configured as a layer or liner, prior to attachment to housing 10010. MP seal 222-55 comprises an MP material such as those described herein. The underside (obscured) of MP seal 222-55 comprises an adhesive that is preferably moisture permeable and adheres to top surface 10008 of housing 10002. MP seal 222-55 can be attached to top surface 10008 of housing as depicted in FIG. 6A-2 and the side view of FIG. 6A-3. MP seal liner 222-55 can be provided to the wearer as part of a kit containing OBD 100-60 and an inserter (not shown). MP seal liner 222-55 can have a removable backing liner on the underside, removal of which by a user exposes the adhesive at which point the user can attach MP seal liner 222-55 to the OBD like a sticker. Attachment can also be performed by the manufacturer prior to provision to a user and performance of the implantation process. In such an example because removal of a sharp from the sensor passageway will leave an aperture in MP seal liner 222-55, the MP seal liner 222-55 can be composed of an elastic material biased to self-close the aperture after removal of the sharp. In another embodiment, MP seal liner 222-55 is attached automatically by an inserter or applicator during the insertion or application process (see, e.g., FIGS. 1T-1 to 1T-6), such as automatically after removal of the sharp from OBD 100.

FIG. 6B-1 is a perspective view depicting an example embodiment of an OBD 100-74 similar to that of FIGS. 2H-1 and 2H-2. In this embodiment OBD 100-70 has a housing 10002 with three vent outlets 131(1), 131(2), and 131(3) in fluid communication with vent passageways 111(1), 111(2), and 111(3) and corresponding vent inlets (obscured). OBD housing 10002 also as a sharp aperture 124 in fluid communication with a sensor passageway 126 and a sensor aperture (obscured) through which sensor 161 extends. FIG. 6A-1 also depicts an MP seal 222-56, configured as a cap, prior to attachment to housing 10002. MP seal cap 222-56 comprises an MP material such as those described herein.

FIGS. 6B-2 and 6B-3 are cross-sectional views of OBD 100-74 and MP seal cap 222-56 taken along line 6B-6B of FIG. 6B-1 and depict MP seal cap 222-56 and OBD housing 10002 before and after assembly, respectively. The cross-sectional views depict the interior or underside of MP seal cap 222-56 in greater detail. MP seal cap 222-56 includes a roof member 2163 having a shape that corresponds to the shape of recessed surface feature 2160 of housing 10002. Recessed surface feature 2160, in this embodiment, is a feature for mating with a container (not shown) in which sensor 161A and a sharp (not shown) can be held during sterilization. Roof member 2163 is connected with a side wall 2164 that extends in an annular manner from a first side of roof member 2163 to give the MP seal cap 222-56 a generally open cylindrical shape. The base (lower) end of side wall 2164 includes a retention feature 2165 configured to interlock with corresponding retention feature 2161 at the base (bottom) of a receiving space 2162 within housing 10002. Retention feature 2165 is in the shape of a detent or lip along side wall 2164, although other shapes can be used. Retention feature 2161 at the base of receiving space 2162 has a shape that is complementary to that of retention feature 2165 such that both features interlock when MP seal cap 222-56 is pushed or pressed into position with side wall 2164 reaching the bottom of receiving space 2162. When MP seal cap 222-56 is secured in place as shown in FIG. 6B-3, roof member 2163 is in a position to permit moisture to permeate from sensor passageway 126 to the external ambient environment. In this embodiment the entirety of MP seal cap 222-56 is composed of an MP material but, in other embodiments, only a portion of MP seal cap 222-56 is composed on an MP material, such as roof member 2163. Any vent passageways that are present in OBD 100-70, such as vent passageways 111(1), 111(2), and 111(3) which are relatively smaller than sensor passageway 126, can be sealed using any of the seals disclosed herein such as those described with respect to FIGS. 3A-1 through 5E-3.

MP seal cap 222-56 can be provided to the wearer as part of a kit containing OBD 100-74 and an inserter (not shown). The user can attach MP seal cap 222-56 to OBD 100-74 by pressing the cap into place manually with his or her hands. Attachment can also be performed by the manufacturer prior to provision to a user and performance of the implantation process. In such an example because removal of a sharp from the sensor passageway 126 will leave an aperture in roof member 2163 of MP seal cap 222-56, the roof member 2163 can be composed of an elastic material biased to self-close the aperture after removal of the sharp. As shown in FIG. 6B-3, the upper flat surface 2166 of roof member 2163 is flush with the top surface 10008 of housing 10002, but in other embodiments surface 2166 can be lower than surface 10008 or can be slightly higher.

The adhesive patch of the embodiments described herein can be composed of a moisture permeable material to act as a moisture permeable seal in certain cases. However, the position of the adhesive patch on the skin facing side of the OBD would not prevent moisture and liquid from entering and exiting the upper portion of the vent and sensor passageways.

Example embodiments of methods of manufacturing (or assembling) an OBD for monitoring an analyte level of a user are provided herein, wherein the OBD includes one or more MP seals. In some embodiments, the method involves injection. In a first step, a housing comprising at least one passageway through the housing is provided. In a second step, a moisture permeable material is injected into the passageway, where the moisture permeable materials forms (e.g., by curing) a seal configured to allow passage of water vapor through the passageway. The housing can be pre-populated with sensor electronics 122 coupled with the housing, or the sensor electronics 122 can be coupled to the housing after injection. An analyte sensor can then be coupled to the sensor electronics 122, either before or after the step of injection. Examples of MP seals formed by injection are described herein, for example, with respect to FIGS. 4A-1 through 4AB-2.

In some embodiments, the method of manufacturing involves adding a preformed moisture permeable seal. The preformed moisture permeable seal can be formed in a mold by curing a liquid moisture permeable material. In a first step, a housing comprising at least one passageway through the housing is provided. In a second step, an MP seal is inserted into the passageway or into a position that covers the passageway, wherein the moisture permeable seal is configured to allow passage of water vapor through the passageway. The MP seal can be inserted into a location having a feature, such as a recess or projection, configured to retain the seal. Alternatively, or additionally, the MP seal can be fixed in the place by the use of adhesive or another form of bonding. Again, the housing can be pre-populated with sensor electronics 122 coupled with the housing, or the sensor electronics 122 can be coupled to the housing after injection. An analyte sensor can then be coupled to the sensor electronics 122, either before or after the step of coupling the electronics 122. Examples of MP seals insertable into the OBD or into a position that covers the passageway are described herein, for example, with respect to FIGS. 5A-1 through 6B-3.

### Foam Seals

The vent apertures, compartments and other venting structures described herein can have a solid foam placed therein in a manner similar to the MP seals and liners described herein. The foam can be open-celled or closed-celled, or a combination thereof. Open-cell foams are generally more permeable to air and moisture vapor than closed-cell foams. Both can be configured to impede significant liquid water ingress while allowing moisture vapor and limited amounts of liquid to pass through. Examples of solid foams usable with the embodiments described herein include but are not limited to: reticulated polyurethane foam; hydrophobic treated open-cell foam; and silicone open-cell foam.

The MP seals of every embodiment described with respect to FIGS. 3A-1 through 5E-3 can be composed of a solid foam instead of an MP material, and these foam structures can be referred to as foam seals. The foam seals can thus have various different shapes and locations of placement as shown in FIGS. 3A-1 through 5E-3, manners of fabrication, and manners of placement within or on OBD 100 as described for the MP seals. As such, FIGS. 3A-1 through 5E-3 need not be replicated herein within images of foam seals substituted for the MP seals.

FIGS. 7A-1 through FIG. 7A-6 are cross-sectional views depicting a passageway 111/126 having example embodiments of a foam seal 333 located therein. References to “foam seal 333” refer collectively to all versions of seal 333-1 through 333-16. Passageway 111/126 has an outlet 131/124 adjacent top surface 10008 and has an inlet 121/112 (vent or sensor) adjacent base (body-facing) surface 10004. Passageway 111/126 can be a vent passageway 111 or a sensor passageway 126 (sensor not shown in FIGS. 7A-1 to 7A-5) of any of the embodiments herein.

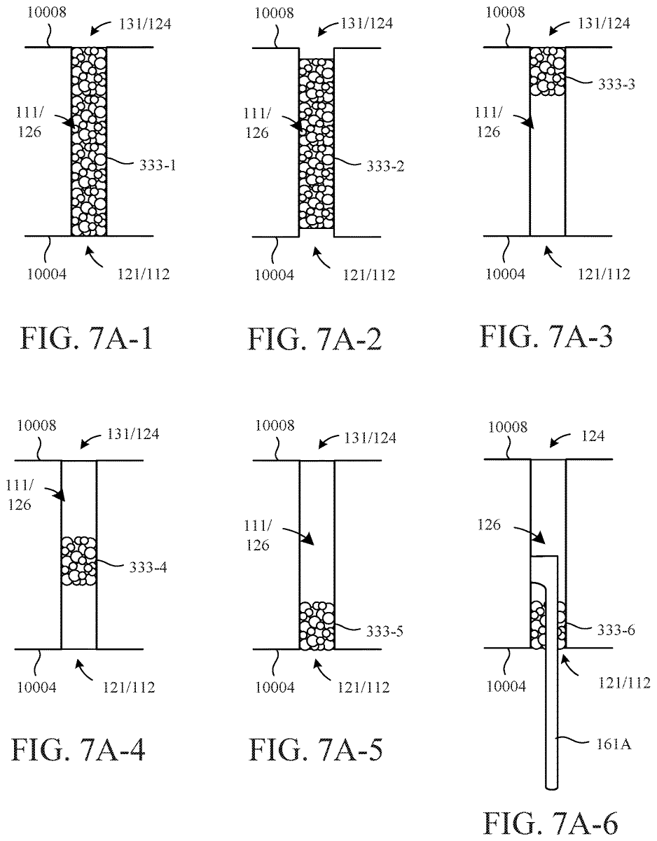

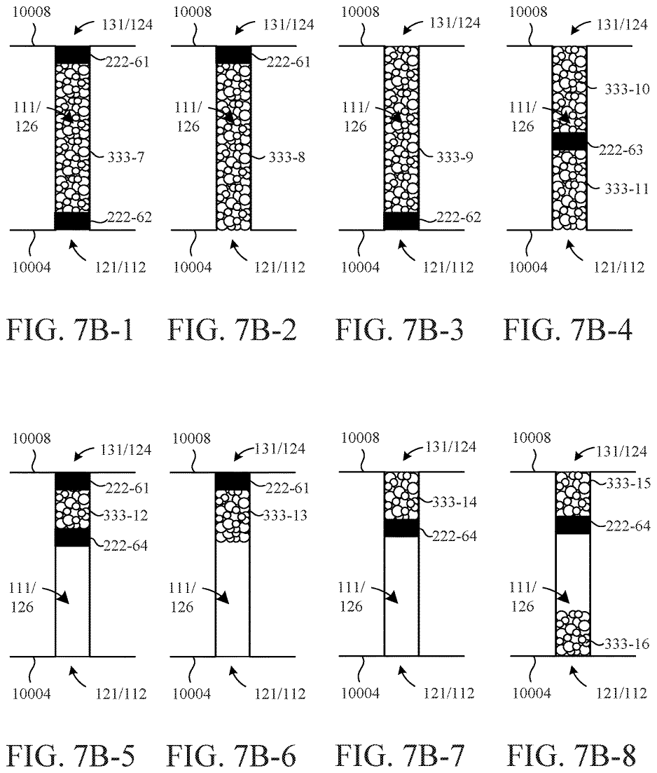

FIG. 7A-1 depicts foam seal 333-1 filling the entire length of passageway 111/126 and being flush with top surface 10008 and base surface 10004. If a channel is present along base surface 10004 (see, e.g., FIGS. 3I-1 and 3I-2) the foam seal 333-1 can extend into the channel. The foam seal 333-1 can extend into contact with any underlying adhesive patch to facilitate moisture transfer therebetween. This embodiment can beneficially transfer moisture the entire length of passageway 111/126. FIG. 7A-2 depicts an example embodiment of foam seal 333-2, which is here configured to extend most of the (but not the entire) length of the passageway 111/126, stopping short of both of top surface 10008 and base surface 10004, to allow room for swelling when filled with moisture. In other embodiments foam seal 333 can stop short of either of surfaces 10008 and 10004.

FIGS. 7A-2, 7A-3, and 7A-4 depict example embodiments of foam seal 333 in various positions within passageway 111/126. In these embodiments, foam seal 333 extends approximately 25% of the length of passageway 111/126. Other lengths of the foam seal 333 can be employed like with the MP seals described herein. In FIG. 7A-3, seal 333-3 is in an upper position within passageway 111/126 adjacent to outlet 131/124. In FIG. 7A-4, seal 333-4 is in an intermediate position within passageway 111/126, midway between outlet 131/124 and inlet 121/112. In FIG. 7A-5, seal 333-5 is in a lower position within passageway 111/126 adjacent to inlet 121/112.

As mentioned, passageway 111/126 can be a vent passageway or a sensor passageway of any of the embodiments herein. The embodiments of FIGS. 7A-1 through 7A-5 can be vent passageways 111 or sensor passageways 126 according to any of the embodiments herein, while FIG. 7A-6 depicts an example embodiment of foam seal 333-6 in sensor passageway 126. Sensor 161A extends through foam seal 333-6 and away from inlet 121/112. A similar configuration can be used with sensor 161B.

The MP seals 222 of all the embodiments described herein can be used in combination with one or more foam seals 333. OBD 100 can have a first (vent or sensor) passageway having only one or more MP seals 222 and a second (vent or sensor) passageway having only one or more foam seals 333. A vent or sensor passageway can have one or more MP seals 222 and one or more foam seals 333 therein.

FIGS. 7B-1 through 7B-8 are cross-sectional views depicting example embodiments of passageway 111/126, which can be a vent or sensor passageway of any of the embodiments herein, having at least one MP seal 222 and at least one foam seal 333 therein. The foam and MP seals can be in contact with each other as shown in these figures, or they can be spaced apart with an air gap existing therebetween. If in contact the seals can be joined by an adhesive, such as a moisture permeable adhesive. In the embodiments of FIGS. 7B-1 through 7B-4, the combination of seals 222 and 333 extends the entire length of passageway 111/126, whereas in the embodiments of FIGS. 7B-5 through 7B-8, the combination of seals 222 and 333 extends only a part of the length of passageway 111/126.

In the embodiment of FIG. 7B-1, a first MP seal 222-61 is located in an upper position within passageway 111/126 adjacent outlet 131/124, a second MP seal 222-62 is located in a lower location adjacent inlet 121/112, and a foam seal 333-7 is located between the MP seals 222-61 and 222-62. In this embodiment, each MP seal 222-61 and 222-62 can prevent ingress of substantial liquid while permitting moisture vapor to move therethrough and the foam seal 333-7 can act as a conduit if moisture builds up between MP seals 222-61 and 222-62.

In the embodiment of FIG. 7B-2, a first MP seal 222-61 is located in an upper position within passageway 111/126 adjacent outlet 131/124 and a foam seal 333-8 is located beneath MP seal 222-61 extending to inlet 121/112. In this embodiment, MP seal 222-61 can prevent ingress of substantial liquid from outside OBD 100 while permitting moisture vapor to move therethrough and the foam seal 333-8 can act as a conduit to transfer moisture that enters passageway 111/126 through inlet 121/112.

In the embodiment of FIG. 7B-3, a first MP seal 222-62 is located in a lower position within passageway 111/126 adjacent inlet 121/112 and a foam seal 333-9 is located above MP seal 222-62 extending to outlet 131/124. In this embodiment, MP seal 222-62 can prevent ingress of substantial liquid from beneath OBD 100 while permitting moisture vapor to exit therethrough and the foam seal 333-9 can act as a conduit to transfer moisture that enters passageway 111/126 out by evaporative forces acting at outlet 131/124.

In the embodiment of FIG. 7B-4, a first foam seal 333-10 is located in an upper position adjacent outlet 131/124, a second foam seal 333-11 is located in a lower position adjacent inlet 121/112, and an MP seal 222-63 is located in an intermediate location between foam seal 333-10 and foam seal 333-11. In this embodiment, the foam seals 333-10 and 333-11 can act to transfer moisture within passageway 111/126 through either inlet 121/112 or outlet 131/124 and MP seal 222-63 can prevent substantial liquid from outlet 131/124 from passing to inlet 121/112.

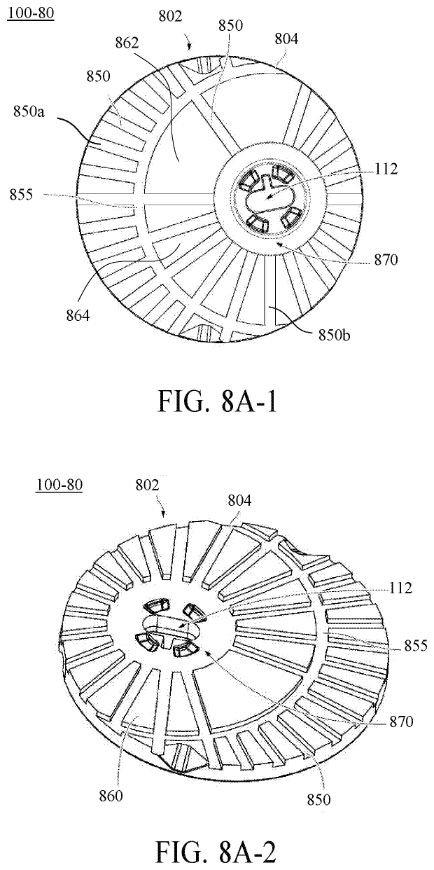

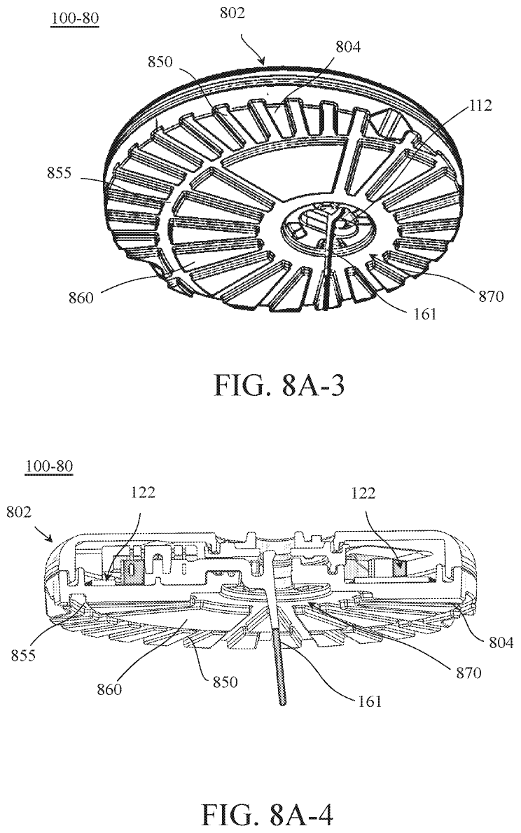

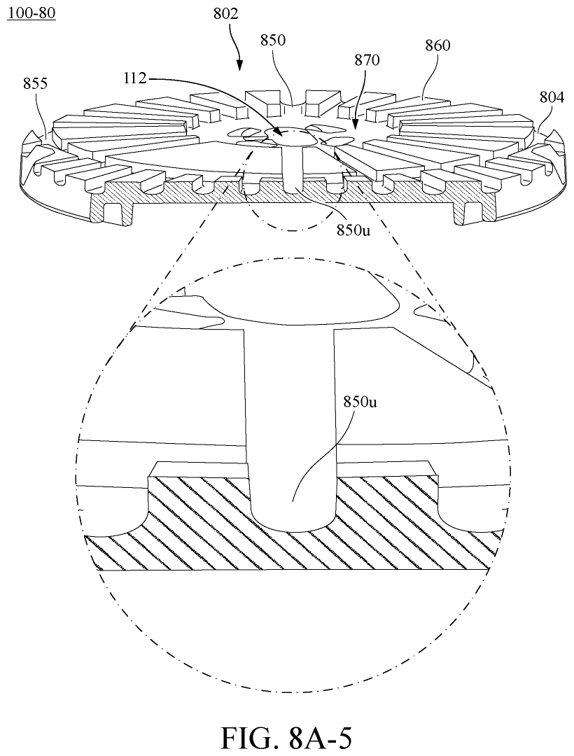

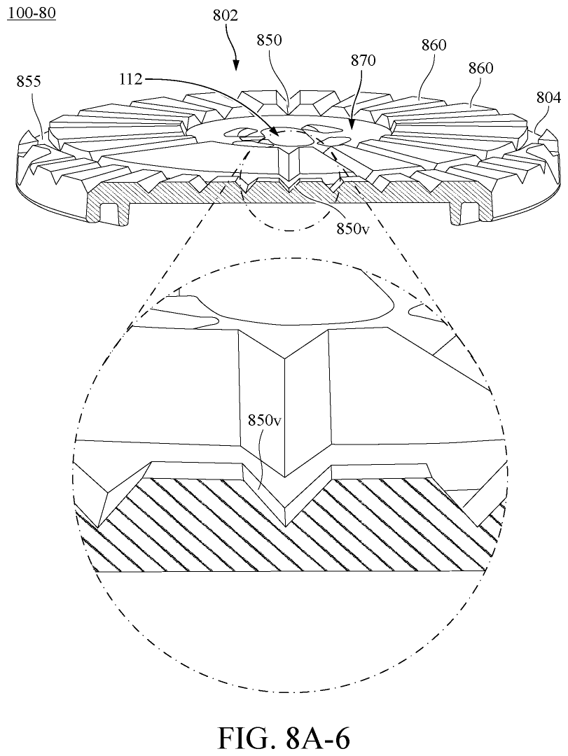

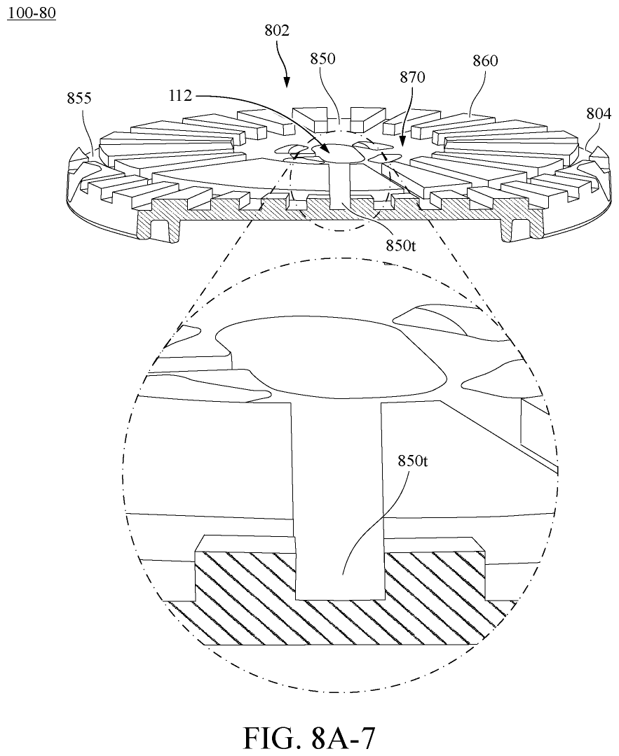

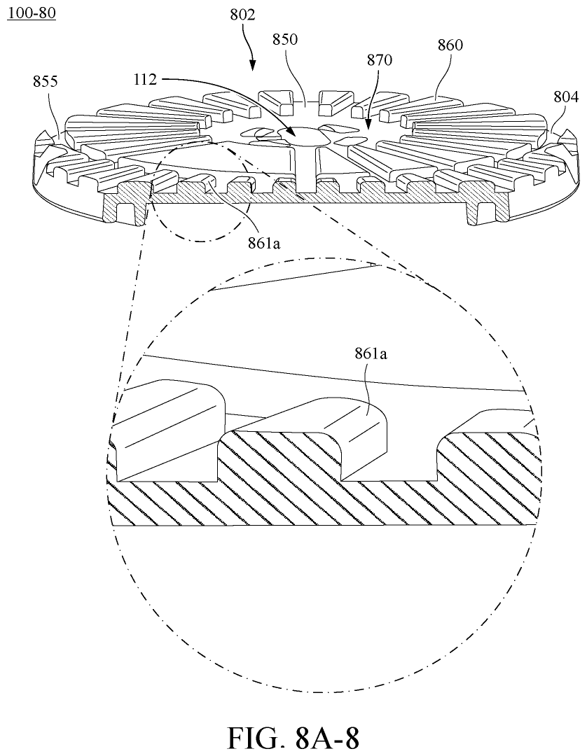

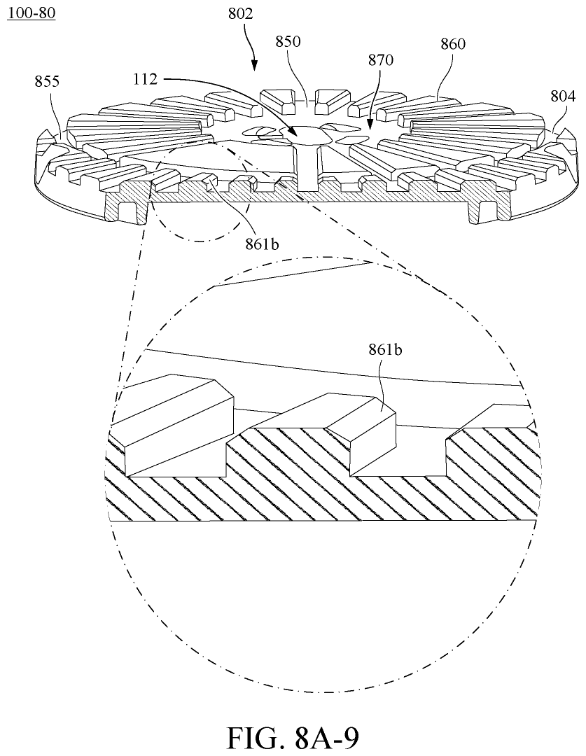

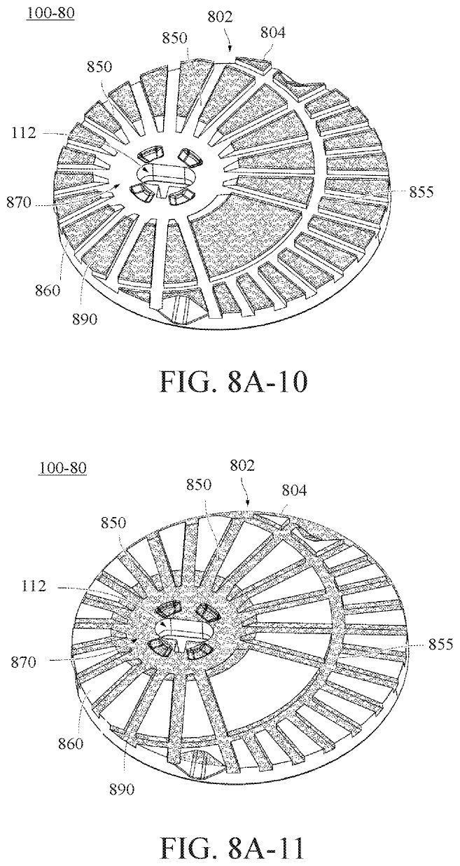

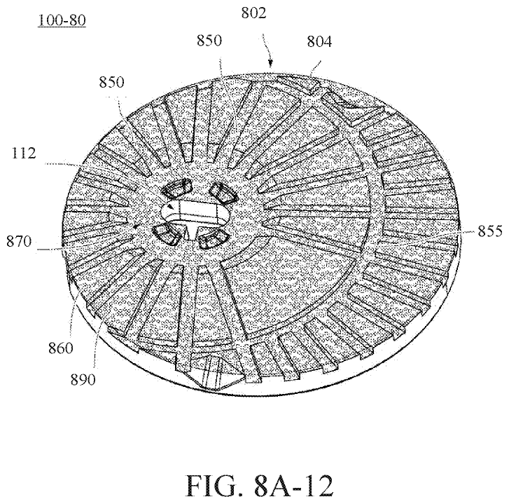

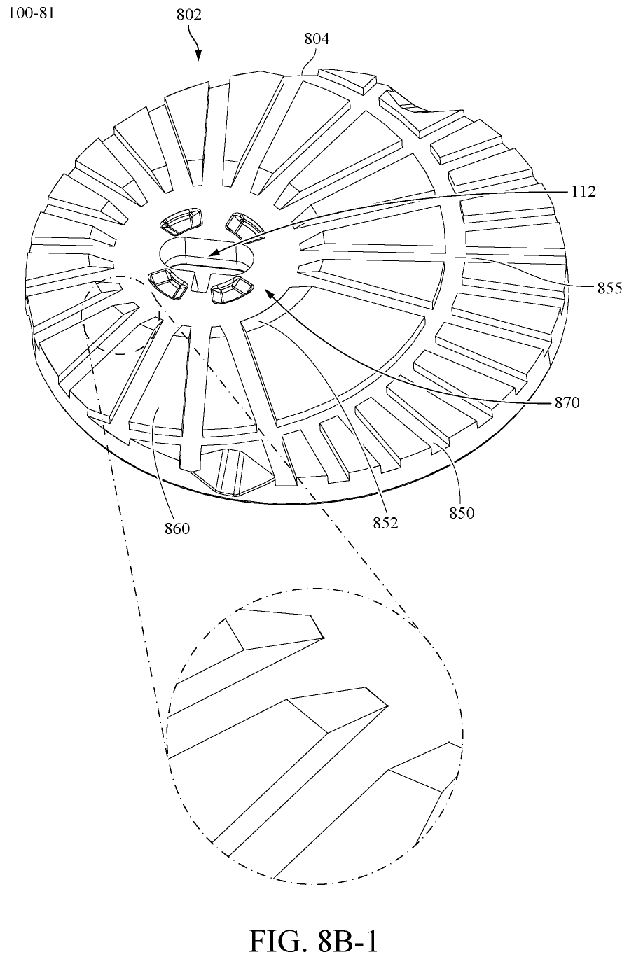

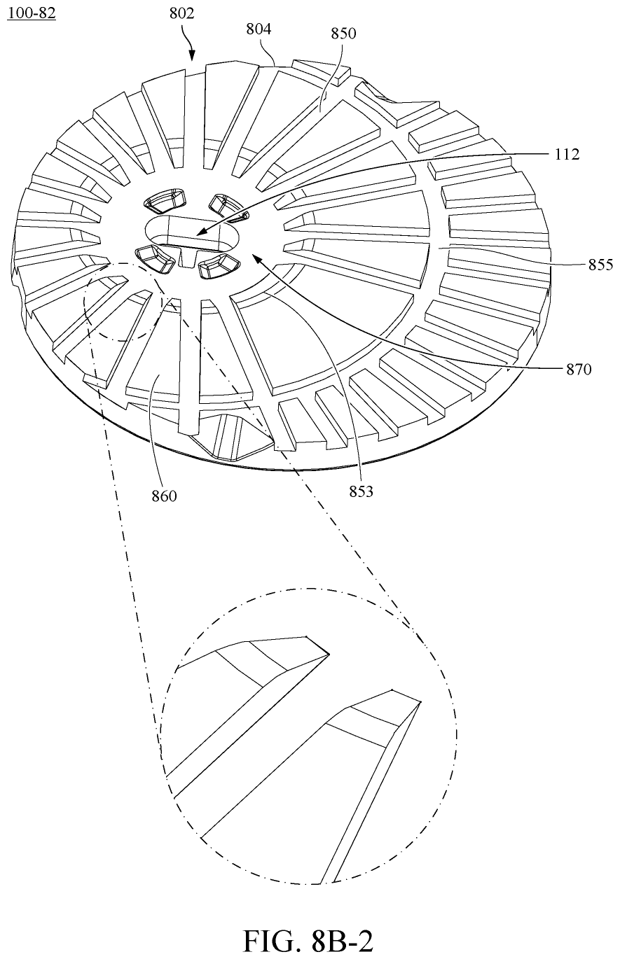

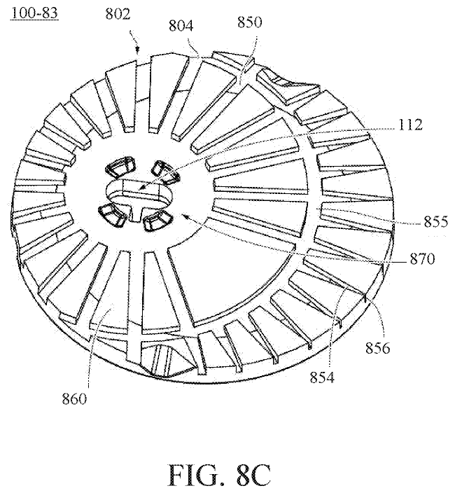

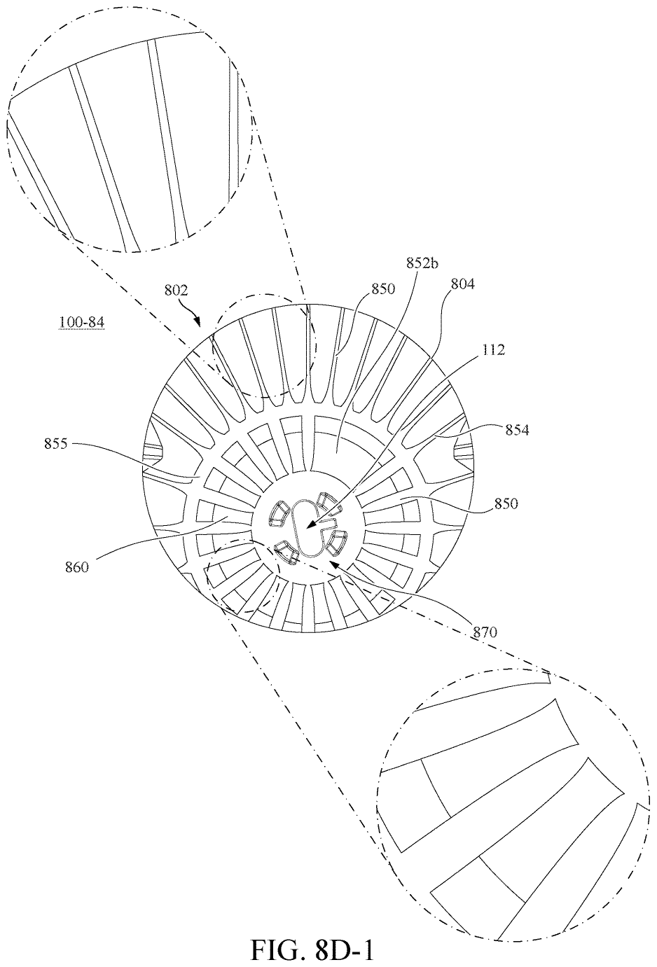

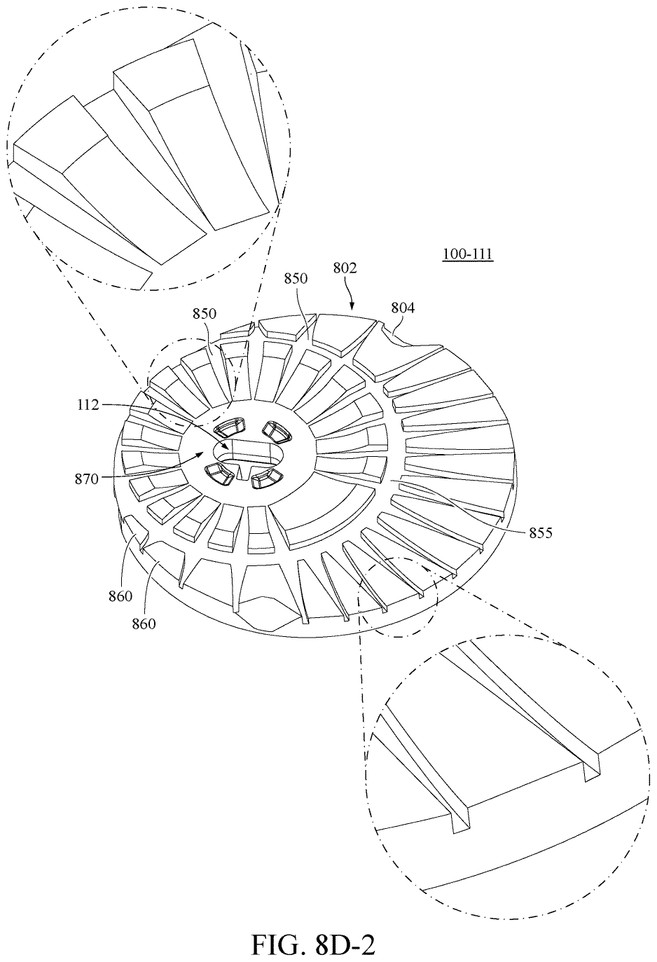

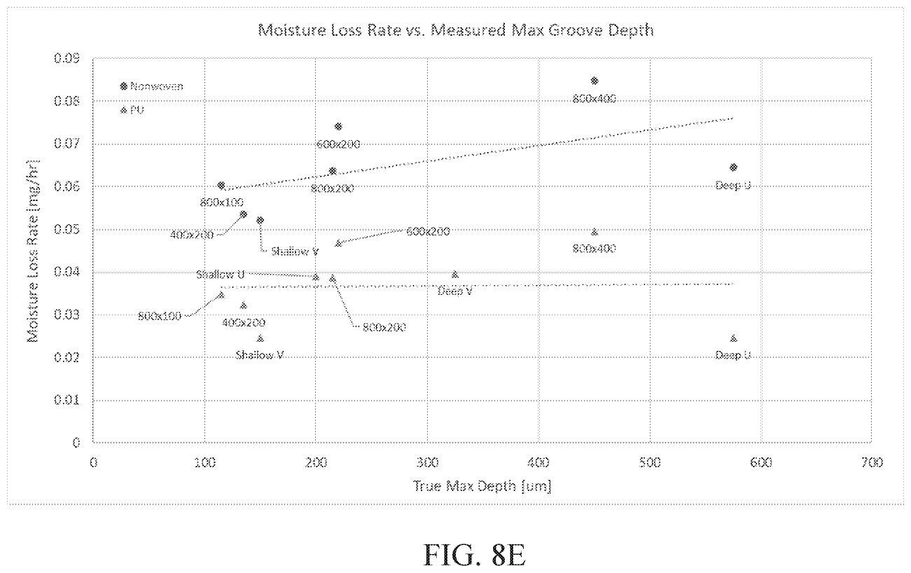

Embodiments having similar configurations but lesser overall lengths as the embodiments of FIGS. 7B-1 through 7B-4 are depicted in FIGS. 7B-5 through 7B-8, respectively. In the embodiments of FIGS. 7B-5 through 7B-8, the seals are located in a generally upper position within passageway 111/126 near outlet 131/124; however, in other embodiments the seals can be located in a lower position near inlet 121/112 or in an intermediate (middle) position within passageway 111/126. The seals can also be spaced apart in different upper, intermediate, and lower locations. In the embodiment of FIG. 7B-5, a first MP seal 222-61 is located in an upper position within passageway 111/126 adjacent outlet 131/124, a foam seal 333-12 is located directly under MP seal 222-61 extending approximately 25-35% of passageway 111/126, and a second MP seal 222-64 is located directly under foam seal 333-12. In the embodiment of FIG. 7B-6, a first MP seal 222-61 is located in an upper position within passageway 111/126 adjacent outlet 131/124 and a foam seal 333-13 is located beneath MP seal 222-61 extending approximately 25-35% of the length of passageway 111/126. In the embodiment of FIG. 7B-7, a first MP seal 222-64 is located in a lower position within passageway 111/126 adjacent inlet 121/112 and a foam seal 333-14 is located above MP seal 222-64 approximately 25-35% of the length of passageway 111/126. In the embodiment of FIG. 7B-8, a first foam seal 333-15 is located in an upper position adjacent outlet 131/124, an MP seal 222-64 is located directly underneath foam seal 333-15, and a second foam seal 333-16 is located at a lower position spaced apart from MP seal 222-64, such that together the seals occupy approximately 50% of the length. While the embodiments of FIGS. 7B-5 through 7B-7 are described as extending approximately 25-35% of the length of passageway 111/126, the seals can occupy greater lengths, such as 35-50% or 35-75%, or can occupy smaller lengths if desired for the application, such as 5-15% or 15-25%

These embodiments of passageways 111/126 can each be configured with one or more features, such as a groove, ridge, slot, projection, or recess, formed by the wall of the passageways to assist in securing the foam seal and prevent dislodgement during and after curing (see, e.g., those features described with respect to the embodiments of FIGS. 4A-1 through 5E-3).

The foam seals 333 can be formed in solid shape prior to installation into the vent and/or sensor passageways or other location (e.g., pre-molded or pre-formed). Alternatively the foam seals can be placed within those passageways by injection of the foam material, while in liquid form, directly into the desired location within the passageway. The foam material can adhere to the wall of the passageway and becomes fixed in position within the passageway as it cures into solid form. One or more features, such as a groove, ridge, slot, projection, or recess, can be formed by the wall of the passageways to assist in securing the injected foam material and prevent dislodgement during and after curing. Foam seals placed through injection can have shapes that conform to the adjacent surfaces. A generally cylindrical passageway can result in a foam seal shape that is also generally cylindrical. Passageways of different shapes will generally result in similar shapes for the foam seals.

### Fibrous Seals

The vent apertures, compartments and other venting structures described herein can have a fibrous material placed therein in a manner similar to the MP seals, MP liners, and foam seals described herein. In contrast to the examples of moisture permeable fibrous materials described with respect to moisture permeable seals, these fibrous materials can be liquid permeable and allow movement of liquid therethrough and can have a regular or irregular lattice of strands that facilitate capillary action movement of that liquid. The fibrous material can be configured to impede significant liquid water ingress while allowing moisture vapor and limited amounts of liquid to pass through. Examples of fibrous materials include natural fibers and synthetic fibers such as a polymer, glass, and carbon fibers. The fibrous material can be held together by virtue of the lattice structure itself or can be supplemented with an adhesive to assist in maintaining the overall structure (e.g., a composite). The fibrous material can be hydrophilic to improve capillary action.

As with the solid foam seals, the MP seals of every embodiment described with respect to FIGS. 3A-1 through 5E-3 can be composed of a fibrous seal instead of an MP material, and these fibrous structures can be referred to as fibrous seals. The fibrous seals can thus have various different shapes and locations of placement as shown in FIGS. 3A-1 through 5E-3, manners of fabrication, and manners of placement within or on OBD 100 as described for the MP seals.

A fibrous seal of similar shape can be substituted for the foam seals of every embodiment described with respect to FIGS. 7A-1 through 7B-8 (i.e., the foam seal omitted and replaced with a fibrous seal). As such, these figures need not be replicated herein with images of fibrous seals in place of the foam seals. The fibrous seals are generally preformed in the sealing structure and inserted into location within the vent passageway or compartment.

The embodiments of solid foam seals and fibrous seals described herein, when used in combination with a moisture permeable seal, can assist in achieving unidirectional moisture flow through the vent passageway or other venting structure, e.g., moisture flow that proceeds predominantly in the direction from vent inlet to vent outlet. For example, the solid foam or fibrous material can draw liquid and/or moisture vapor from the base area of OBD 100, which has the adhesive patch and is in contact or close proximity with skin and the insertion site, and hold that liquid and/or moisture near or in contact with a moisture permeable seal for an extended duration of time, which allows the liquid and/or moisture to slowly migrate through the moisture permeable seal through the vent outlet and out of the housing in the direction of the ambient environment. In contrast, liquid and moisture coming from the ambient environment on the opposite side of the vent outlet is only in relatively shorter duration of contact with the moisture permeable seal in many use case situations, with the longest being bathing, showering, or swimming. The moisture permeable seal can be configured so that such relatively shorter duration contact, on the order of 15 minutes or less, is not sufficient for significant moisture to migrate through the seal in the opposite direction from the ambient environment towards the vent inlet. Thus, the embodiments can achieve unidirectional flow in the intended direction.

In the embodiments of FIG. 2A-1 through FIG. 2G, FIG. 2I-5, FIG. 2J-1 through FIG. 2K-12, and FIG. 3A-1 through FIG. 3I-2, the vent passageways and sensor electronics can be positioned such that the one or more vent passageways go through sensor electronics (e.g., as described with respect to FIGS. 2H-1 and 2H-2). Alternatively, the one or more vent passageways can be routed, and the sensor electronics 122 can be positioned, such that the one or more vent passageways do not pass through the sensor electronics. The sensor electronics 122 can be positioned away from the vent passageways, either side-to-side (left or right), up or down (e.g., above vent passageways 111 in the embodiments of FIGS. 2E-1, 2E-2, 2F-1, 2F-2, 3E-1, 3E-2, 3F-1, and 3F-2 or below vent passageways 111 in the embodiments of FIGS. 2B-2, 3B-3, and 3B-4).

As described, e.g., with respect to FIGS. 2K-1 through 2K-12, OBD 100 can have multiple vent passageways, in each case in combination with a sensor passageway if such is needed to operate with a partially implantable sensor (e.g., between 2 and 90 vent passageways plus a sensor passageway), and all embodiments of MP seals can be used in each such vent and sensor passageway of OBD 100. Likewise, all embodiments of foam seals can be used in each such vent and sensor passageway of OBD 100. Likewise, all embodiments of fibrous seals can be used in each such vent and sensor passageway of OBD 100.

The embodiments described herein can be utilized to extend the designed wear duration of on-body devices 100. The resulting extended designed wear durations will depend on the actual implementation of OBD 100 and the sensor 160 it operates in conjunction with. For all OBDs 100 (e.g., 100-1 through 100-105) and all systems (e.g., system 10 and systems 10-A through 10-G), the designed wear durations can in various embodiments be (or can be up to) 1 day, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23, days, 24 days, 25 days, 26 days, 27 days, 28 days, 29 days, 30 days, 31 days, 45 days, 60 days, 90 days, and/or other amounts therebetween those listed or longer. The designed wear durations can be in ranges such as 1 to 7 days, 7 days or greater, 8 to 14 days, 14 days or greater, 15 to 30 days, 30 days or greater, 31 to 45 days, 7 to 60 days, and/or 45 days or greater.

### Additional Moisture Egress Features

The following section describes various embodiments of an OBD 100 having additional features that facilitate the egress of moisture (e.g. from perspiration, showering, swimming, or other environmental exposure) or from an area adjacent the housing, or beneath the housing in use. Such features can take the forms of channels, or grooves, and surface textures that can direct moisture away from the area adjacent the housing. This section includes a description of OBDs 100-80 through 100-100 referring to FIGS. 8A-1 to 14B. Any combination of channels and surface textures as described with respect to OBDs 100-80 to 100-100 in FIGS. 8A-1 to 14B can be combined in a single embodiment of an OBD 100.

OBDs 100-80 to 100-100 can be applied to a user using any of the applicators or inserters 60 described herein in the section titled “INSERTERS AND APPLICATORS”. The inserter 60 may comprise a distal end configured to be positioned on the skin of the subject before advancement of the on body electronics unit 122 and the sharp from a proximal position. The on body electronics unit 122 and the sharp may be configured to advance along a linear path when advancing from the proximal position to the distal position. The sharp may be configured to pierce the skin of the subject at an angle substantially perpendicular to the skin of the subject.

### Channels

### On-Body Device 100-80

Referring to FIGS. 8A-1 to 8A-12, an OBD 100-80 is shown according to an embodiment. OBD 100-80 is similar to the various OBDs described herein, such as OBD 100-1 of FIGS. 1B-1 to 1B-3, and may include corresponding features. Various alternative features described in relation to OBD 100-1 may similarly be applied to OBD 100-80 in a corresponding manner. Similarly, the features described in relation to OBDs 100-1′ to 100-74 may be applied to OBD 100-80. OBD 100-80 may be used as part of the analyte monitoring system 10. For example, OBD 100-80 may be used with at least one receiver unit 20.

OBD 100-80 includes a number of channels 850, 855 formed in a base surface 804 of the housing 802. The channels 850, 855 are formed as trenches within the base surface 804, such that proximal portions 860 (or bank or platform portions) are present between the channels. In other words, the proximal portions 860 define the channels 850, 855.

By virtue of the channels 850, 855 providing defined moisture pathways to the periphery of the housing 802, the housing 802 is adapted for moisture egress, and thereby reduces moisture accumulation under the OBD housing 802. Reducing moisture in this area improves breathability and thereby skin health, which reduces the shedding of dead skin cells. Accordingly, adhesive performance may be enhanced, and the likelihood of the adhesive patch peeling away may be reduced. In other words, delamination time of an adhesive patch 106 is increased, and longer wear durations are possible.

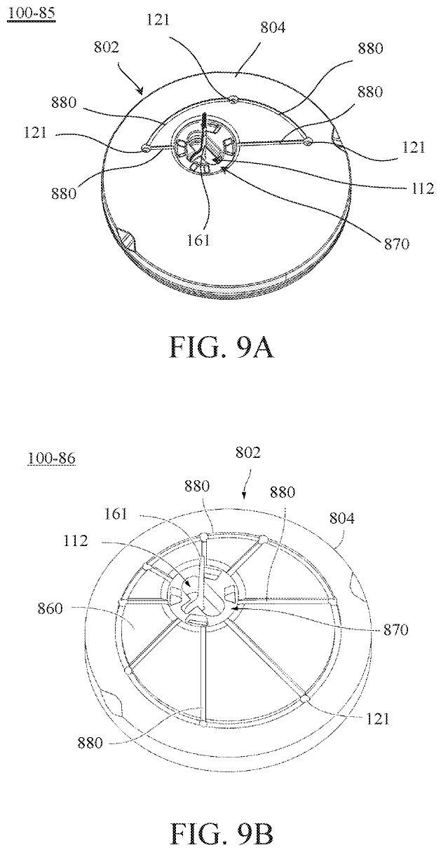

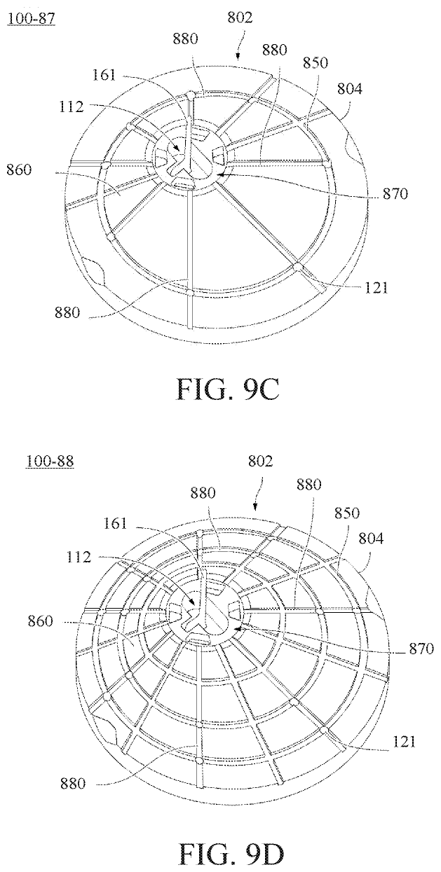

The channels 850, 855 (all or a subset thereof), may have any suitable cross-sectional profile, such as being U-shaped 850u, V-shaped 850v, or three-sided 850t (such as square or rectangular), as shown respectively in FIGS. 8A-5, 8A-6 and 8A-7. Additionally, a fillet 861a or a chamfer 861b may be provided at the intersection between side walls of the channels 850, 855 and the base surface 804 of the housing 802, as shown respectively in FIGS. 8A-8 and 8A-9.

A first type of channel 850 (herein referred to as first channels or radial channels) are provided in a radial direction. In some cases, the first channels 850 extend from a first region 870 of the base surface 804, in which a sensor aperture 112 is located, to a peripheral edge of the housing 802. Accordingly, a first end of the first channels 850 is arranged at the first region 870, and a second end is arranged at a peripheral edge of the housing 802. Having channels extending to the periphery provides a direct exit route for moisture to escape from under the OBD 100. Moreover, channels 850 connecting the periphery to a central region can allow moisture egress away from critical areas, such as a sensor insertion site located in the first region 870. Other first channels 850 extend over a partial radius, such as between the peripheral edge of the housing 802 and an intermediate point, such as an intersection with another channel (e.g. channel 855). Other first channels 850 extend over a partial radius, between the first region 870 and an intermediate point, such as an intersection with another channel (e.g. channel 855).

The first region 870 may be a recessed region of the base surface 804, such as a circular recessed region, or may otherwise be a region from which the first (radial) channels emanate. As depicted in FIGS. 8A-1 and 8A-2, the first region 870 can be provided offset from the centre of the of the base surface 804 of the housing 802, such as offset along an axis of symmetry. Alternatively, the first region 870 and sensor aperture 112 could be provided in the centre of the housing 802, or in the centre of the base surface 804 of the housing 802. Alternatively, if the device is used with a wholly implantable sensor 162 or a wholly ex vivo sensor 163, the first region 870 may be omitted. In other words, if a partially implantable sensor 161 is not used, a recessed first region 870 may not be required. Channels 850 may extend directly from sensor aperture 112 such that there is no significant first region between channel 850 and aperture 112.

The housing may take any shape, such as the housing shapes depicted in FIGS. 1N3-1N20. For instance, the peripheral edge of the base surface 804 of the housing 802 may have a shape comprising a circular shape. The peripheral edge of the base surface 804 of the housing 802 may have a shape comprising a truncated circular shape. The peripheral edge of the base surface 804 of the housing 802 may have a shape comprising a semi-circular shape. The peripheral edge of the base surface 804 of the housing 802 may have a shape comprising an elliptical shape. The peripheral edge of the base surface 804 of the housing 802 may have a shape comprising an annular shape. The peripheral edge of the base surface 804 of the housing 802 may have a shape comprising a teardrop shape. The peripheral edge of the base surface 804 of the housing 802 may have a shape comprising a crescent shape. The peripheral edge of the base surface 804 of the housing 802 may have a shape comprising a triangular shape. The peripheral edge of the base surface 804 of the housing 802 may have a shape comprising a pill shape. The peripheral edge of the base surface 804 of the housing 802 may have a shape comprising a dumbbell shape. The peripheral edge of the base surface 804 of the housing 802 may have a shape comprising a square shape. The peripheral edge of the base surface 804 of the housing 802 may have a shape comprising a rectangular shape. The peripheral edge of the base surface 804 of the housing 802 may have a shape comprising a trapezium shape. The peripheral edge of the base surface 804 of the housing 802 may have a shape comprising a parallelogram shape. The peripheral edge of the base surface 804 of the housing 802 may have a shape comprising a pentagon shape. The peripheral edge of the base surface 804 of the housing 802 may have a shape comprising a hexagon shape. The peripheral edge of the base surface 804 of the housing 802 may have a shape comprising a heptagon shape. The peripheral edge of the base surface 804 of the housing 802 may have a shape comprising an octagon shape. The peripheral edge of the base surface 804 of the housing 802 may have a shape comprising a nonagonal shape. The peripheral edge of the base surface 804 of the housing 802 may have a shape comprising a decagonal shape. The peripheral edge of the base surface 804 of the housing 802 may have a shape comprising a hendecagonal shape. The peripheral edge of the base surface 804 of the housing 802 may have a shape comprising a dodecagonal shape. The peripheral edge of the base surface 804 of the housing 802 may have a shape comprising a combination of any of the aforementioned shapes. The peripheral edge of the base surface 804 may have any other shape. The peripheral edge of the base surface 804 of the housing 802 may comprise one or more indentations and/or one or more protrusions or lobes. Such shapes may include sharp and/or rounded corners/vertices, and straight and/or curved edges (with constant or variable radii of curvature).

The first region 870 and/or sensor aperture 112 may be provided at a centroid of the shape of the base surface 804 of the housing 802. This may be along an axis of symmetry of the shape, or along an axis that is perpendicular to an axis of symmetry of the shape and in the plane of the base 804. Alternatively, the offset may be along both an axis of symmetry of the shape and an axis that is perpendicular to the axis of symmetry of the shape.

Referring to FIG. 8A-3, a sensor 161 extends through the aperture 112 in the first region 870 in use. The sensor may be configured in any manner as described herein in the section titled “ANALYTE SENSOR”. For instance, instead of a partially implantable sensor 161, the sensor may be a wholly implanted sensor 162 or a wholly ex vivo sensor 163. The sensor 161 may be glucose sensor comprising a proximal portion configured to electrically couple with electronics 122 and a distal portion having a glucose-responsive enzyme. The distal portion is configured to be positioned in a bodily fluid of the subject's body and to sense glucose in the subject's bodily fluid in a subcutaneous layer.

Referring to FIG. 8A-4, the sensor is configured to provide a signal indicative of a level of an analyte to electronics 122 retained within the housing 802. The electronics 122 may be configured in any manner described above in the section titled “SENSOR ELECTRONICS”. The electronics 122 may comprise a processor, memory, a power supply, and wireless communication circuitry configured to wirelessly communicate data indicative of a glucose level. The on-body electronics unit, or electronics 122, may be configured to be disposed after a single use.

A second type of channel 855 (herein referred to as second channels or arcuate channels) is also provided in a base surface 804 of the housing 802. Although one second channel 855 is depicted in FIGS. 8A-1 to 8A-12, any number of second channels 855 may be provided such as two channels, three channels or four channels. Multiple such second channels 855 may be provided in a concentric or parallel arrangement. Alternatively, multiple second channels may be provided in non-parallel arrangements and may intersect. The second channel 855 is provided such that both ends are arranged at a peripheral edge of the housing 802. This arrangement allows the second channel 855 to intersect and interconnect the first (radial) channels 850. Accordingly, it may also be considered that there are radial channel portions 850a, or third channels 850a, extending between the periphery of the housing 802 and the second channel 855, and radial channel portions 850b, or third channels 850b, between the second channel 855 and the first region 807, as depicted in FIG. 8A-1.

In other words, a first end of a first channel 850 (or radial channel 850), may be arranged near, or at, the centre of the base of the housing 802 and a second end of the radial channel 850 may be arranged at or near the periphery of the housing 802. In this way, the first channel 850 may create a cross-type intersection with the second channel 855. Additionally or alternatively, a first end of a radial channel 850a may be arranged at an intersection with a second channel 855, such as in a T-junction type arrangement or in a cross-junction type arrangement, and a second end of the radial channel 850a may be arranged at or near the periphery of the housing 802. Additionally or alternatively, a first end of a radial channel 850b may be arranged at an intersection with a second channel 855, such as in a T-junction type arrangement or in a cross-junction type arrangement, and a second end of the radial channel 850a may be arranged at or near the centre of the base of the housing 802. Additionally or alternatively, a portion of a first, radial channel 850 may extend between, and optionally intersect, two or more second channels 855. Overall, this may allow for various patterns of channels, including various grid arrangements, which may be regular or irregular.

In this way, the channels 850, 855 can provide multiple pathways by which moisture can exit at the periphery of the OBD 100-80. As such, if one pathway becomes blocked (e.g. due to skin cells or liquid droplets), alternative exit pathways for moisture are provided. The second channel 855 may be provided in a particular curved configuration, such as an arc of a circle or an ellipse, having the aperture 112 as the focal point of the arc. Each end of the curved configuration or arc of the second channel 855 may terminate at the periphery of the base. The curved configuration or arc may be greater than 90 degrees and less than 360 degrees (e.g., a full circle or ellipse). The curved configuration or arc may be greater than 180 degrees and less than 360 degrees as shown in FIG. 8A-1. The curved configuration or arc may be greater than 210 degrees and less than 360 degrees. Additionally or alternatively, the second channel could be formed of two or more different portions each having different curved geometric properties, such as with different focal points and/or different radii of curvature.

The proximal portions 860, which can be integral portions of the base surface 804 of the housing 802, define a contact surface onto which an adhesive patch 106 (not shown) may be applied. Accordingly, a greater contact surface area can permit stronger adhesive coupling between the adhesive patch 106 and the housing 802. With channels 850, 855 provided, the contact surface area may be around 30% to 80%, or more preferably between 50% and 60%, or more preferably around 55%.

The proximal portions 860 of any embodiment of OBD 100 described herein can have various shapes and sizes that differ from each other. The shapes and sizes may depend on the configuration of the on-body device 100, including its size, shape, and placement of sensor aperture 112. Here, on-body device 100-80 has a proximal portion 862 that is relatively larger than other proximal portions of various shapes and sizes, such as proximal portion 864. Proximal portions 862 and 864 have locations therein that are generally at the same or similar radial distance from sensor aperture 112 although they can be located at different radial distances. Proximal portions 862 and 864 may be bordered by channels 850, 855 on at least three sides, with the fourth side located adjacent another channel 850, 855, a periphery of the base surface, or a first region 870 (such as a region having sensor aperture 112). Here, proximal portion 862 is bounded by two radial channels 850 and curved channel 855, and first region 870.

Proximal portion 862 may be the largest proximal portion defined by the surface area of its contact surface. In this example, proximal portion 862 has a contact surface with a surface area that is three times (300%) or more than that of proximal portion 864. In another embodiment the on-body device housing can have one or more proximal portions 862 with a contact surface with a surface area that is 1.25 times (125%) as large or more than that of at least one other proximal portion 864. In yet another embodiment the on-body device housing can have one or more proximal portions 862 with a contact surface with a surface area that is two times (200%) as large or more than that of at least one other proximal portion 864. In one embodiment, the on-body device housing can have at least one proximal portion with a contact surface with a surface area that is 5% or greater of the total area of the base surface 804 facing the skin (e.g., the portions of base surface 804 visible in FIG. 8A-1, thus omitting channel sidewalls). In another embodiment, the on-body device housing can have at least one proximal portion with a contact surface with a surface area that is 7% or greater of the total area of the base surface 804 facing the skin. In another embodiment, the on-body device housing can have at least one proximal portion with a contact surface with a surface area that is 10% or greater of the total area of the base surface 804 facing the skin. In these embodiments, the contact surfaces can range from 5%, 7%, or 10% up to 30% in one embodiment, 25% in another embodiment, 20% in yet another embodiment, and 15% in still another embodiment. Proximal portion 862 does not extend to the peripheral edge of the base, but a similarly sized proximal portion adjacent to portion 862 (to the upper right of portion 862 in FIG. 8A-1) does extend to the peripheral edge. A relatively larger contact surface provides more surface area by which adhesive can adhere to the adhesive patch 106 (not shown) and thus improve securement between the on-body device housing and the adhesive patch. All embodiments of OBD 100 described herein (e.g., OBDs 100 and 100-1 through 100-105) may have one or more such proximal portions 862.

Referring to FIGS. 8A-10, 8A-11 and 8A-12, OBD 100-80 may also be provided with a moisture permeable material 890, which can be in a nonwoven or woven form. The moisture permeable material 890 may take any form as described herein under the section titled “VENT SEALS”. In particular, as depicted in FIG. 8A-10, the moisture permeable material 890 may be provided on the proximal portions 860. As illustrated, the moisture permeable material 890 is provided on all of the proximal portions 860. Alternatively, the moisture permeable material may be provided on only some of the proximal portions 860. Alternatively, as depicted in FIG. 8A-11, the moisture permeable material 890 may be provided within the channels 850, 855. As illustrated, the moisture permeable material 890 is within (and may fill) the channels 850, 855, but not on the proximal portions 860. Instead, the moisture permeable material 890 could be provided within only some of the channels 850, 855, and/or may partially fill the channels 850, 855. Additionally or alternatively, as depicted in FIG. 8A-12, the moisture permeable material 890 may be provided both in the channels 850, 855 and on the proximal portions 860. In other words, the moisture permeable material 890 may coat the whole base surface 804 of the housing 802. Alternatively, the moisture permeable material 890 may coat a portion of the base surface 804 of the housing 802.

### On-Body Device 100-81

Referring to FIG. 8B-1, OBD 100-81 is shown according to an embodiment. OBD 100-81 may be the same as OBD 100-80 of FIGS. 8A-1 to 8A-12, and may include the same features except as explained below. The alternative features described in relation to OBD 100-80 may similarly be applied to OBD 100-81 in a corresponding manner. In turn, since OBD 100-80 is similar to OBD 100-1, features of OBD 100-1 may be applied to OBD 100-81. Similarly, the features described in relation to OBDs 100-1′ to 100-74 may be applied to OBD 100-81.

In this embodiment, OBD 100-81 corresponds to OBD 100-80, except that the first channels 850 are provided with variable depth. In particular, as depicted in FIG. 8B-1, the first channels 850 include a portion with reduced depth as the first channels progress towards the first region 870 in the base surface 804 of the housing 802. The depth of each of the first channels 850 decreases as each channel progresses toward the first region 870 of the base 804 until the first channel bottoms meets the first surface 804. Advantageously, this configuration avoids the creation of an abrupt recessed space at the first region 870 that could lead to induration of the skin. Such configurations can also promote the directing of moisture away from the first region 870 and toward the periphery, and can minimise any compromises to structural integrity resulting from the channels 850.

The depth of a channel can be defined as the distance between a bottom of a channel (distal to the base surface 804) and the base surface 804 of the housing 802, along a plane that is normal (perpendicular) to the plane of the base surface 804 of the housing 802.

The reducing depth, as depicted, takes the form of a tapered or beveled edge 852 at the periphery of the first region 870 on the proximal portions 860 around the first region 870. Alternatively, a reducing depth could take the form of a stepped reduction in depth. Tapering channels 850 to zero depth allows them to smoothly merge with the base surface 804 and first region 870. A gradual and continuous depth variation avoids the creation of an abrupt edge or recess that could lead to induration of the skin.

In the foregoing, the length of a respective channel may be considered the full extent of the length, such as between the periphery of the base of the housing 802 and the first region 870, even if the respective channel includes intersections with other channels. The length of a channel portion, or channel section, may be considered the length of a respective channel between intersections with other channels, or between the periphery and an intersection point, or between the first region and an intersection point. The portion of the length of the first channels 850 that reduce in depth can be 1% or greater of the length of the respective channel (or channel portion), 2% or greater of the length of the respective channel (or channel portion), 3% or greater of the length of the respective channel (or channel portion), 5% or greater of the length of the respective channel (or channel portion), 8% or greater of the length of the respective channel (or channel portion), 10% or greater of the length of the respective channel (or channel portion), 15% or greater of the length of the respective channel (or channel portion), 20% or greater of the length of the respective channel (or channel portion), 25% or greater of the length of the respective channel (or channel portion), 30% or greater of the length of the respective channel (or channel portion), 50% or greater of the length of the respective channel (or channel portion), 75% or greater of the length of the respective channel (or channel portion), or 95% or greater of the length of the respective channel (or channel portion).

In turn, the depth of the first channels 850 can increase as they progress towards the peripheral edge of the housing 802. In this way, the maximum depth can be at the periphery, providing increased capacity for removal of moisture at the outlet of the channels. Increased capacity at the outlet of the channels 850 can be desirable, as additional moisture can be routed through the channels 850 in their outermost portions due to moisture directed therein from second channels 855.

The second channels 855 may also be provided with a varying depth profile in a corresponding manner to the first channels 850.

### On-Body Device 100-82

Referring to FIG. 8B-2, OBD 100-82 is shown according to an embodiment. OBD 100-82 may be the same as OBD 100-80 of FIGS. 8A-1 to 8A-12, and may include the same features except as explained below. The alternative features described in relation to OBD 100-80 may similarly be applied to OBD 100-82 in a corresponding manner. In turn, since OBD 100-80 is similar to OBD 100-1, features of OBD 100-1 may be applied to OBD 100-81. Similarly, the features described in relation to OBDs 100-1′ to 100-74 and 100-81 may be applied to OBD 100-82.

OBD 100-82 corresponds to OBD 100-81 of FIG. 8B-1, except that the tapered edge, providing a reducing depth at the first region 870, takes the form of a radiused sloping edge 853.

### On-Body Device 100-83

Referring to FIG. 8C, OBD 100-83 is shown according to an embodiment. OBD 100-83 may be the same as OBD 100-80 of FIGS. 8A-1 to 8A-12, and may include the same features except as explained below. The alternative features described in relation to OBD 100-80 may similarly be applied to OBD 100-83 in a corresponding manner. In turn, since OBD 100-80 is similar to OBD 100-1, features of OBD 100-1 may be applied to OBD 100-83. Similarly, the features described in relation to OBDs 100-1′ to 100-74, 100-81 and 100-82 may be applied to OBD 100-83.

Referring to FIG. 8C, OBD 100-83 is shown according to an embodiment. OBD 100-83 corresponds to OBD 100-80, except that the first channels 850 are provided with variable width. The width of the channel is the distance between opposing sidewalls of the channel in the plane of the base surface 804, and measured along a direction that is generally perpendicular to the sidewalls.

In particular, as depicted in FIG. 8C, the first channels 850 include a portion 854 with reduced width at the periphery of the housing 802. The width may reduce or taper along the entire length of the channels 850, or along a portion of the length of the channels, by the periphery of the housing 802. As a result, the channels 850 can terminate at the periphery of the housing 802 in the shape of a slit 856.

Additionally, as depicted in FIG. 8C, the first channels 850 may also deepen along portion 854 as the width of the channels reduces, or tapers, toward the periphery of the base. In this way, a relatively consistent cross-sectional area may be maintained along the length of the channels 850, so that moisture transport rates are not adversely impacted by the narrowing width.

The channels 850 can include different width profiles along different lengthwise spans thereof. For instance, two lengthwise spans may have different widths, such as a narrower width nearer the periphery of the base of the housing 802, and the transition between the two width profiles may be smooth or stepwise. Each lengthwise span may have a constant width, or the width may vary according to a desired profile.

The second channels 855 may also vary in width in a corresponding manner as described in relation to the first channels 850, such as by including a portion with reduced width at one or both ends of the second channels 855, at the periphery of the housing 802 (not shown in FIG. 8D). Thus, the second channels 855 may also include a tapering portion and/or a slit termination 856. The second channels 855 may also be provided with a different width variation profile to the first channels 850.

A narrowing or tapering width of the channels 850, 855 maximizes contact surface area at the periphery of the base surface 804 of the housing 802 for coupling with an adhesive patch 106 (not shown), and ultimately with the user. This maximizes adhesion surface area at the periphery, to reduce the risk of peel away commencing from the peripheral edge. It is desirable to avoid the commencement of any peeling away, as a peeling edge can propagate readily, particularly as the exposed adhesive can catch on clothing or other materials, leading to additional shear forces being applied to the OBD 100-83 and adhesive patch 106.

Providing for different widths along the channels 850, 855 also enables improved and tailored moisture removal. A wider channel permits greater moisture removal rates, but reduces surface area of the base available for adhesion. Thus, providing for varying widths allows for efficient moisture removal without excessively sacrificing adhesion.

### On-Body Device 100-84

Referring to FIG. 8D-1 and 8D-2, OBD 100-84 is shown according to an embodiment. OBD 100-84 may be the same as OBD 100-80 of FIGS. 8A-1 to 8A-12, and may include the same features except as explained below. The alternative features described in relation to OBD 100-80 may similarly be applied to OBD 100-84 in a corresponding manner. In turn, since OBD 100-80 is similar to OBD 100-1, features of OBD 100-1 may be applied to OBD 100-84. Similarly, the features described in relation to OBDs 100-1′ to 100-74, 100-81 to 100-83 may be applied to OBD 100-84.

Referring to FIG. 8D-1, OBD 100-84 is shown according to an embodiment. OBD 100-84 corresponds to OBD 100-80, except that the first channels 850 are provided with variable width and variable depth. In particular, a portion 854 having variable width is provided toward the periphery of the base 804 of the housing 802. The width varies in the portion 854 in a non-linear manner, which may be, for example, parabolic or exponential. Other portions of the channels 850 may also have variable width. As will be appreciated, the width may vary in various non-linear or linear manners, or in any combinations thereof, to create regular or irregular patterns. Non-linear profiles may create smoother transitions between channels, such as channels 850 and 855, which may enable more efficient guiding of moisture. Additionally, the depth may vary within the portion 854 so as to maintain an approximately consistent cross-sectional area, as described above in the context of OBD 100-83. As shown in FIG. 8D-2, the variable width portion 854 may also terminate in a slit shape 856.

Referring to FIG. 8D-2, a portion having variable depth 852b is also provided, toward the centre of the base of the housing 802, and around the first region 807. The depth of this portion 852b may also vary in a non-linear manner, such as, for example, parabolically or exponentially. The portion 852b may, at the same time as varying in depth, also vary in width. In this way, a channel 850, 855 may vary in width and depth along its length, both in a linear and non-linear manner, along different portions thereof. As will be appreciated, the depth may also vary in various non-linear or linear manners, or in any combinations thereof, to create regular or irregular patterns. Such variable profiles enable tailoring moisture removal characteristics.

### On-Body Device Channel Dimensions

Each of the first channels 850 and/or each of the second channels 855 in any of the on-body devices disclosed herein may have dimensions as set out in the following section.

In particular, each of the first channels 850 and/or each of the second channels 855 may have a width that is between 400 μm and 1000 μm. Preferably, the width is between 600 μm and 900 μm. More preferably, the width is between 700 and 800 μm. More preferably, the width is around 800 μm. Such dimensions may be applicable to any OBD implementation as described herein.

Each of the first channels 850 and/or each of the second channels 855 may have a (maximum) depth that is greater than 50 μm. Preferably, the depth may be between 100 μm and 400 μm. More preferably, the depth may be between 100 μm and 200 μm. More preferably, the depth may be around 100 μm. Such dimensions may be applicable to any OBD implementation as described herein.

A relatively shallow channel, around 100 μm, ensures that the depth or height of the housing does not need to be excessively large so as to accommodate the depth of the channels formed therein. Whilst a deeper channel can promote greater moisture egress, this must be balanced against an increase in required device height/depth to accommodate such channels. A tall device may increase the likelihood of knocks, which reduce wear duration. A shallow channel can also improve adhesion, as a portion of an adhesive patch may flex inwards and contact a portion of the base of a channel. A wide channel can be selected to promote greater moisture removal rates, more than compensating for any loss of moisture removal rates resulting from a shallow channel. In particular, a wide channel, of around 800 μm, can enhance moisture removal rates without requiring a larger device housing to accommodate the channels. Of course, a maximum adequate surface area must remain between the channels to permit adhesion.

The combination of a wide and shallow channel, of around 800 μm wide and 100 μm deep, allows for unexpectedly high moisture removal rates, without requiring a deep/tall housing to accommodate the channels, which would negatively affect wear duration by increasing the chance of knocks. In particular, as shown in FIG. 8E, the inventors have found that the depth of the channels can be reduced to a shallow depth of just 100 μm with unexpectedly little impact on performance (moisture loss rate), particularly when compared against the 200 μm depth and 400 μm depth data points (with 800 μm width, for example). Accordingly, a width to depth ratio of around 8:1 has been found to be particularly effective in terms of improvements in wear duration (balancing factors of moisture removal rates, device height, and adhesive contact surface area). The inventors have also found that rectangular shaped channels perform best, by providing maximum channel volume, though U-shaped and V-shaped channels also perform well.

### On-Body Devices 100-85 Through 100-88

Referring to FIGS. 9A-D, an OBDs 100-85 through 100-88 are shown according to an embodiment. OBDs 100-85 through 100-88 are similar to the various OBDs described herein, such as OBD 100-1 of FIGS. 1B-1 to 1B-3, and may include corresponding features. Various alternative features described in relation to OBD 100-1 may similarly be applied to OBDs 100-85 through 100-88 in a corresponding manner. Similarly, the features described in relation to OBDs 100-1′ to 100-74 and 100-80 to 100-84 may be applied to OBDs 100-85 through 100-88. OBDs 100-85 through 100-88 may be used as part of the analyte monitoring system 10. For example, OBDs 100-85 through 100-88 may be used with at least one receiver unit 20.

Referring to FIG. 9C, OBD 100-85 corresponds to OBD 100-80, for example, except that the channels are provided in an alternative arrangement, and vent passageways are provided in the housing 802. The vent passageways may correspond to the vent passageways described herein in the section titled “VENT PASSAGEWAY”. A moisture permeable seal may also be provided in the vent passageways, in a corresponding manner as described herein in the section titled “VENT SEAL”.

The vent passageways are provided with vent inlets 121 on the base surface 804 of the housing 802. The vent inlets take the form of circular apertures, but may take any other appropriate shape. On an opposing surface of the housing are vent outlets (not shown), which are fluidically connected to the vent inlets 121 through the housing 802, which allows the passage of water vapor from the base surface 804 to an upper surface of the housing. Accordingly, the vent inlets 121 and vent outlets are connected by a vent passageway, which may extend through the electronics 122. The vent inlets 121 may be aligned with the vent outlets along an axis perpendicular to the base surface 804, or they may be offset. The vent inlets 121 are each spaced away from the periphery of the housing 802. Any number of vent inlets 121 may be provided. As shown in FIG. 9, three vent inlets are provided 121. Two of the vent inlets are provided such that they are arranged on a straight line with the sensor aperture 112, though other arrangements are possible.

A number of channels of a fourth type 880 (herein referred to as fourth channels) are provided to interconnect vent inlets 121 and the sensor aperture 112. The fourth channels 880 may correspond in their features to the first and second channels 850, 855 described herein, except in respect of their end connections. Some of the fourth channels 880 interconnect vent inlets 121 and the first region 870 comprising the sensor aperture 112. In other words, one end of a given fourth channel 880 is arranged at a vent inlet 121, in fluid communication therewith, and another end of the fourth channel 880 is arranged at the first region 870, in fluid communication with the sensor aperture 112.

Some other channels of the fourth channels 880 interconnect vent inlets 121. For these channels 880, one end is arranged at a vent inlet 121, in fluid communication therewith, and the opposing end is arranged at another vent inlet 121, in fluid communication therewith. A channel 880 being in fluid communication with a vent inlet 121 may be achieved by providing the vent inlet 121 in the (distal) bottom of a channel 880. Alternatively, as depicted in FIG. 9A, the vent inlets 121 may be wider than the channels 880.

Alternatively, the fourth channels may be arranged to interconnect some or all of the vent inlets without connecting to the first region 870 or sensor aperture 112. This allows channels to interconnect multiple vent inlets, creating a network feeding moisture to the vents for removal.

Referring to FIG. 9B, OBD-86 corresponds to OBD 100-85, but includes additional channels 880 across the surface of the base. The channels 880 connect a series of vent inlets 121 in a closed loop, or circle, and additional channels 880 are arranged radially, as spokes of the closed loop. Such an arrangement provides for additional moisture routing options across the base surface of the housing 802.

Referring to FIG. 9C, OBD 100-87 corresponds to OBD 100-86, but includes additional radial channels 880. Some radial channels 880 may intersect a vent inlet 121, and other radial channels 880 may not intersect a vent inlet 121. Some radial channels also extend to the peripheral edge of the base of the housing 802, such that moisture may exit at the periphery, as well as via the vent inlets 121, and via the sensor aperture 112.

Referring to FIG. 9D, OBD-88 corresponds to OBD 100-87, but includes additional radial channels 880, additional concentric circular channels 880, and additional vent inlets 121. As for OBD 100-87, some radial channels 880 may intersect a vent inlet 121, and other radial channels 880 may not intersect a vent inlet 121. Some radial channels also extend to the peripheral edge of the base of the housing 802, such that moisture may exit at the periphery, as well as via the vent inlets 121, and via the sensor aperture 112. The pattern of channels 880 may be a regular or irregular grid pattern, including vent inlets at some or all intersections of the channels 880, and/or vent inlets positioned along the channels but not at intersections of the channels 880, and/or vent inlets 121 positioned outside of channels (such that they do not interact with channels). As will be appreciated, any appropriate pattern of channels 880 in combination with vent inlets 121 may be used.

The fourth channels may be provided in the same manner, and with corresponding features, as the first and second channels 850, 855 described above in connection with OBDs 100-80, 100-81, 100-82, 100-83 and 100-84, such as in respect of their shape, depth and width profiles. For instance, tapering depth may also be provided where the fourth channels 880 connect to the vent inlets 121.

The fourth channels may be straight or curved in shape, and the interconnections may create a closed network for routing moisture on the base surface 804 of the housing 802. Advantageously, this integrates the channels in the base of the housing 802 with through-housing vents (and sensor apertures), providing a combined moisture management system, enabling moisture egress from across the base surface and through the vents. This arrangement avoids a need for moisture outlets at the periphery of the housing, ensuring that the periphery of the base surface 804 of the housing 802 can be an uninterrupted smooth surface, providing maximal surface area for adhesion in the peripheral region where peel risk is greatest.

The network of channels 880 and vent inlets 121 may be arranged in any suitable shape, such as: a line; a circle; an ellipse; an oval; a semi-circle; a triangle; a square; a pentagon; a hexagon; a heptagon; an octagon; or a star. The vent inlets 121 may be provided at vertices of such shapes, and/or along edges (within channels 880) thereof.

### On-Body Device 100-89

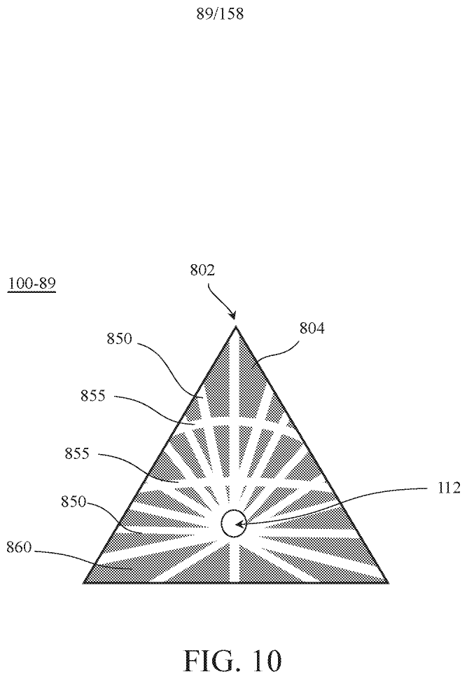

Referring to FIG. 10, an OBD 100-89 is shown according to an embodiment. OBD 100-89 may be the same as OBD 100-80 of FIGS. 8A-1 to 8A-12, and may include the same features, except as explained below. The optional features and alternatives described in relation to OBD 100-80 may similarly be applied to OBD 100-89 in a corresponding manner. In some examples, OBD 100-89 may be similar to OBDs 100-80 through 100-88, and features described in relation to those embodiments may be applied to OBD 100-89.

In this embodiment, OBD 100-89 corresponds to OBD 100-80, except that the housing 802 is triangular in shape. A number of first channels 850 are provided so as to radiate from a sensor aperture 112 that is located in an offset position along an axis of symmetry of the housing. Two arcuate second channels 855 are also provided. The channels 850, 855 may otherwise correspond to those described herein in connection with any of OBD 100-80 through OBD 100-88.

It will be appreciated that alternative patterns of channels may be provided if the sensor aperture 112 is provided in an alternative offset location, such as an offset position that is not along an axis of symmetry. Alternative patterns can also be achieved by rotating and/or translating the positions of each of the second channels 855, and/or by altering the pitch and/or dimensions of the channels 850, 855.

### Surface Texture

### On-Body Devices 100-90 Through 100-95

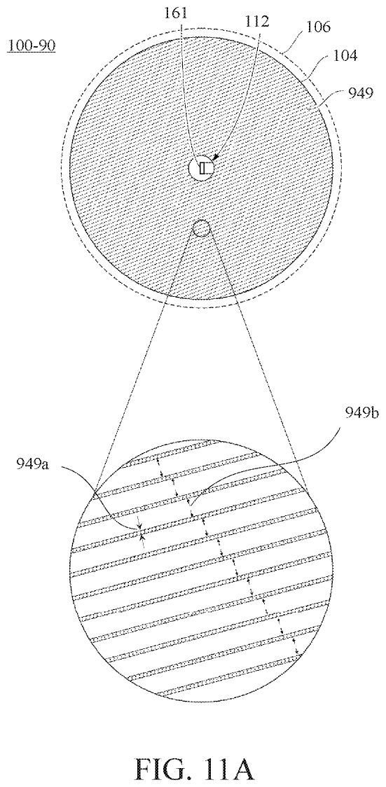

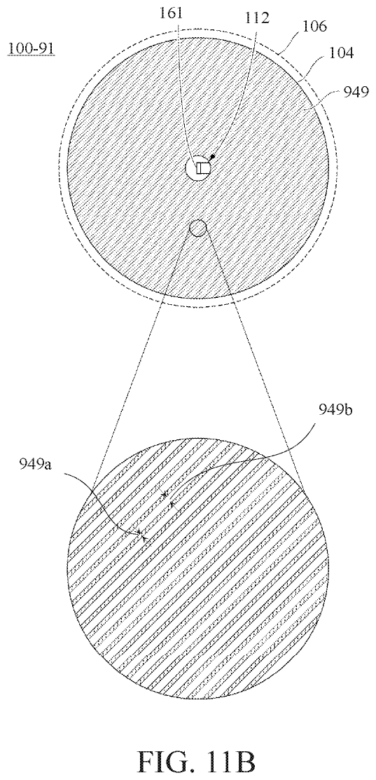

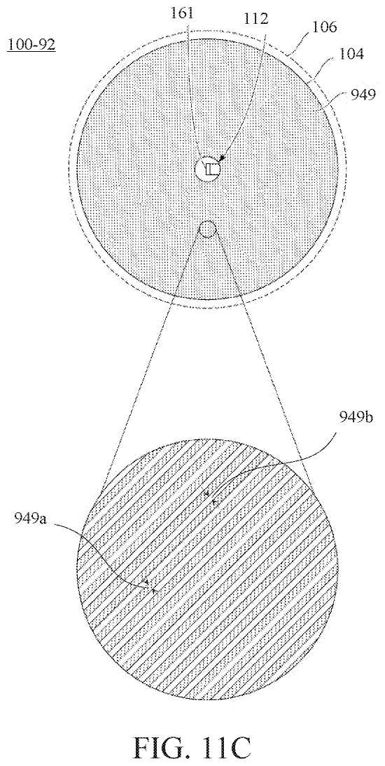

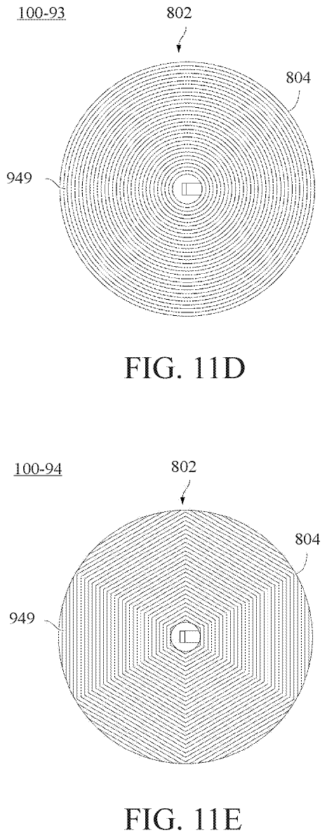

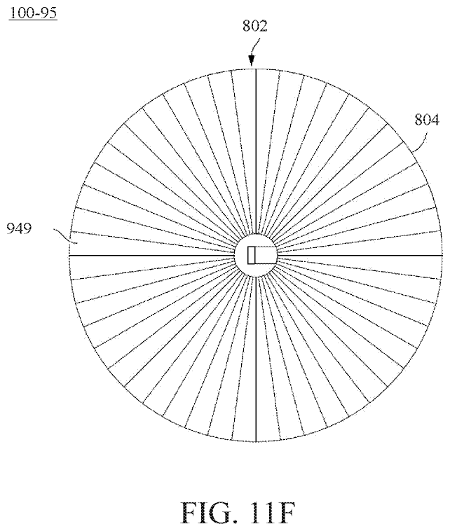

Referring to FIGS. 11A-11F, OBDs 100-90 through 100-95 are shown according to embodiments. OBDs 100-90 through 100-95 may be the same as OBD 100-1 of FIGS. 1B-1 to 1B-3, except that OBDs 100-90 through 100-95 are provided with a surface texture applied to a bottom surface 804 of the housing 802. OBDs 100-90 through 100-95 may otherwise include the same features as OBD 100-1 of FIGS. 1B-1 to 1B-3. The optional features and alternatives described in relation to OBD 100-1 may similarly be applied to OBDs 100-90 through 100-95 in a corresponding manner. In some examples, OBDs 100-90 through 100-95 may be similar to OBDs 100-80 through 100-89, and features described in relation to those embodiments may be applied to OBDs 100-90 through 100-95.

FIGS. 11A-11C are bottom views showing OBDs 100-90 through 100-92 with the adhesive patch removed (for the purposes of illustration) to show the exposed base surfaces 804 of each of OBDs 100-90 through 100-92. A directional (anisotropic) surface texture 949 is applied to the exposed base surface 804 of the housing 802. Surface texture 949 can enhance adhesion of an adhesive patch 106 to the base of the housing by effectively increasing the surface area and providing mechanical interlocking features. The surface texture 949 may also facilitate the movement or moisture across the base surface 804 of the housing 802, by providing pathways to promote channeling and evaporation.

The directional surface texture 949 is formed of a series of generally aligned (e.g. parallel or aligned within around 15 degrees of a common direction) grooves across the base surface 804. The grooves are distinct from the channels as described herein, in that the grooves are shallow surface features. For instance, the average channel depth may be at least 10 μm greater than the surface texture 949 average depth, or preferably at least 50 μm greater, or more preferably at least 100 μm greater, or more preferably at least 150 μm greater. The directional surface texture 949 may be applied to the base surface 804 by a laser process, by electrical discharge machining, or by a wire brush process, for example.

The grooves of the surface texture 949 may have a depth of around 40 μm, a width of around 100 μm, and a pitch of around 300 μm. Alternatively, the grooves of the surface texture 949 may have a depth of around 50 μm, a width of around 600 μm, and a pitch of around 850 μm. The arithmetic mean height roughness (Sa) may be between 9 and 18 μm, or between 20 and 22 μm.

Although depicted as being straight in orientation, the surface texture 949 may be curved, circular, elliptical, triangular, square, or any polygonal shape. As such, the surface texture 949 may be applied in a concentric pattern.

As depicted in the zoomed portions of FIGS. 11A through 11C, the surface texture may be provided with different widths and different depths of the grooves, and with different pitch (i.e. the distance between adjacent grooves of the surface texture 949). In FIG. 11A, the surface texture 949 grooves of OBD 100-90 have a width 949a that is relatively small compared to (i.e. smaller than, or significantly smaller than) the separation 949b, or pitch 949b, between adjacent surface texture grooves. As such, the surface texture grooves are relatively sparse. In FIG. 11B, the surface texture 949 grooves of OBD 100-91 have a width 949a that is similar to, or the same as, the separation 949b, or pitch 949b, between adjacent surface texture grooves. In FIG. 11C, the surface texture 949 grooves of OBD 100-92 have a width 949a that is relatively large compared to (i.e., larger than, or significantly larger than) the separation 949b, or pitch 949b, between adjacent surface texture grooves. Accordingly, the surface texture may be applied with different coarseness or depth to provide the desired properties in terms of adhesion and moisture routing across the surface. The coarseness, width or pitch may also vary across different portions of the base surface to tailor the properties as desired. For instance, more grooves (smaller pitch) may be desired near the peripheral edge of the base to enhance surface area, and thus adhesion strength, in that region. Although straight lines are depicted, curved or wavey surface texture grooves/lines may be used.

Referring to FIGS. 11D through 11F, the directional surface texture 949 may be applied in alternative patterns, other than the straight lines depicted in FIGS. 11A through 11C. In FIG. 11D, the surface texture 949 applied to OBD 100-93 is provided in a pattern of concentric circles. In FIG. 11E, the surface texture 949 applied to OBD 100-94 is provided in a pattern of concentric polygons. A hexagon is shown, although a polygon of any number of sides may be used. In FIG. 11F, the surface texture 949 applied to OBD 100-95 is provided in a pattern of radial lines, with the sensor aperture as the focus. Although shown with consistent pitch, the pitch (and/or width and/or depth) of the grooves may vary to tailor the properties across the surface.

### On-Body Devices 100-96 Through 100-100

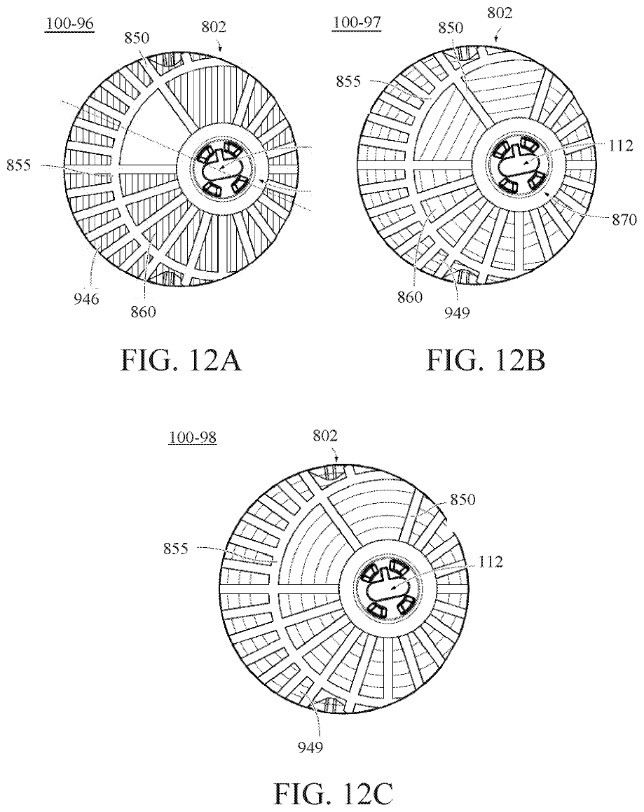

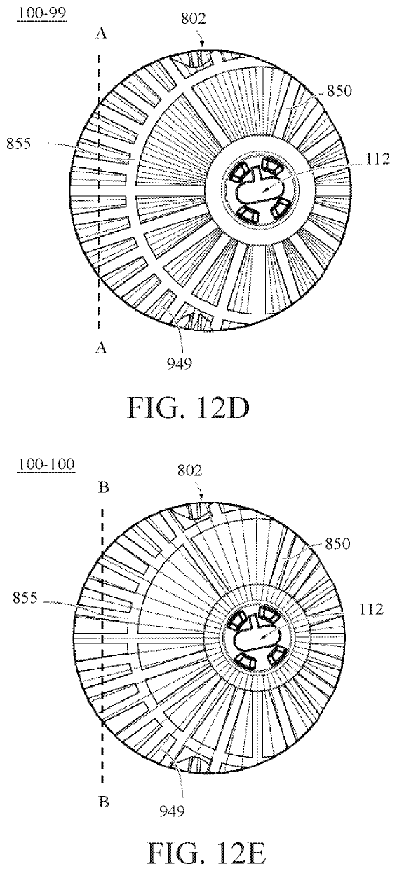

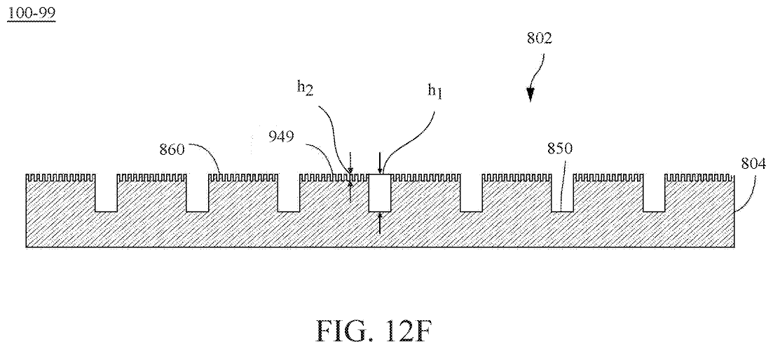

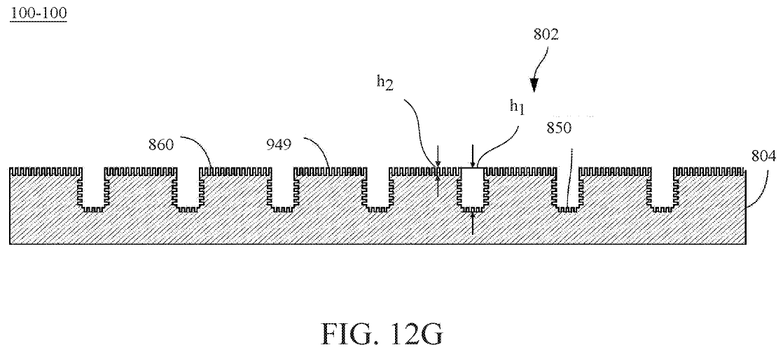

Referring to FIGS. 12A through 12G OBDs 100-96 through 100-100 are shown according to an embodiment. OBDs 100-96 through 100-100 may be the same as OBD 100-1 of FIGS. 1B-1 to 1B-3, and may include the same features, except as explained below. The optional features and alternatives described in relation to OBD 100-1 may similarly be applied to OBDs 100-96 through 100-100 in a corresponding manner. In some examples, OBDs 100-96 through 100-100 may be similar to OBDs 100-80 through 100-95, and features described in relation to those embodiments may be applied to OBDs 100-96 through 100-100.

FIG. 12A depicts a directional surface texture 949, such as the directional surface texture of FIGS. 11A through 11E, as applied to the base surface of an OBD having channels 850, 855, such as in OBD 100-80. Accordingly, the directional surface texture 949 of FIGS. 12A through 12G may have any of the features described herein in connection with FIGS. 11A through 11E. Similarly, the channels 850, 855 may have any of the features described herein in connection with FIGS. 8A-1 through 10.

In OBDs 100-96 through 100-100, as depicted in FIGS. 12A through 12D, the surface texture 949 is provided only on the proximal portions 860 of the base surface 804. The surface texture 949 in these portions assists in routing moisture across the base surface 804 and to the periphery of the housing 802, as well as with the strength of adhesion to the adhesive patch 106 (not shown).

Additionally or alternatively, as shown in FIG. 12B, the surface texture 949 may be applied in a concentric polygonal arrangement. The concentric polygons may centre on the sensor aperture 112. The polygonal arrangement may be selected such that the surface texture 949 is applied so as to be generally perpendicular to the first channels 850. In this way, the surface texture efficiently guides and routes moisture into the channels 850 for removal via the peripheral edge of the housing 802, and/or via vents if provided (not shown). If alternative shapes of housing are used, the concentric polygons may algin with the shape of the periphery of the base surface 804 of the housing 802.

Additionally or alternatively, as shown in FIG. 12C, the surface texture 949 may be applied in a generally concentric circular or elliptical shape. This can have a similar effect of feeding or routing moisture into the channels 850, 855, so that there is a cooperative function between the channels 850, 855 and the surface texture 949.

Additionally or alternatively, as shown in FIG. 12D, and in cross-section in FIG. 12F (along line A-A of FIG. 12D), the surface texture 949 may be applied in a generally radial fashion, such as extending from the first region 870 to the peripheral edge of the housing 802. In this way, the surface texture 949 can provide a direct pathway for routing moisture to the periphery of the housing 802 for removal, to complement the action of the channels 850, 855.

Additionally or alternatively, as shown in FIG. 12E, and in cross-section in FIG. 12G, the surface texture can be applied within the channels 850, 855 (as well as on the proximal portions 860), such as on the base of channels 850, 855, and/or on the sidewalls of the channels 850, 855. This can provide improved routing of moisture within the channels themselves.

The dimensions of the grooves of the directional surface texture 949 may be as is described herein in connection with FIG. 11. The depth (or height) of the surface texture 949 grooves is indicated in FIGS. 12F and 12G as h2. Similarly, the dimensions of the channels 850, 855, may be as is described herein in the section titled “On-body device channel dimensions”. The depth (or height) of the channels 850, 855 is indicated in FIGS. 12F and 12G as h1. The surface texture 949 groove depth, h1, is smaller, or significantly smaller, than the channel depth. In particular, average depth h1 of the channels 850, 855 may be at least 10 μm greater than the surface texture 949 average depth h2, at least 50 μm greater than the surface texture 949 average depth h2, at least 100 μm greater than the surface texture 949 average depth h2, or at least 150 μm greater than the surface texture 949 average depth h2. The surface texture 949 grooves may be V-shaped, U-shaped or three-sided (square or rectangular), or any other suitable shape, in cross-section.

The surface texture 949 may be applied in combinations of patterns, and with variable groove pitch, width and/or depth, as described above in connection with FIGS. 11A-F, across different portions or zones of the base surface 804, to provide a combination of effects.

### On-Body Devices 100-101 Through 100-103

Referring to FIGS. 13A-1 to 13C-2, OBDs 100-101 through 100-103 are shown according to embodiments. OBDs 100-101 through 100-103 may be the same as OBD 100-1 of FIGS. 1B-1 to 1B-3, except that 100-101 through 100-103 are provided with a surface texture applied to a bottom surface 804 of the housing 802. OBDs 100-101 through 100-103 may otherwise include the same features as OBD 100-1 of FIGS. 1B-1 to 1B-3. The optional features and alternatives described in relation to OBD 100-1 may similarly be applied to OBDs 100-101 through 100-103 in a corresponding manner. In some examples, OBDs 100-101 through 100-103 may be similar to OBDs 100-80 through 100-100, and features described in relation to those embodiments may be applied to OBDs 100-101 through 100-103.

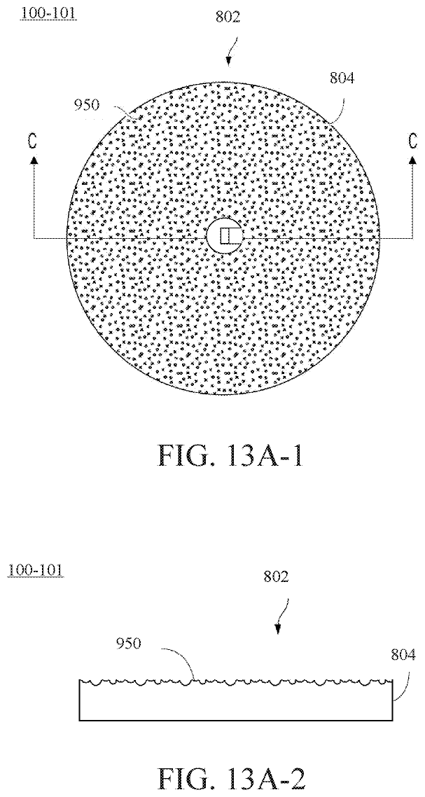

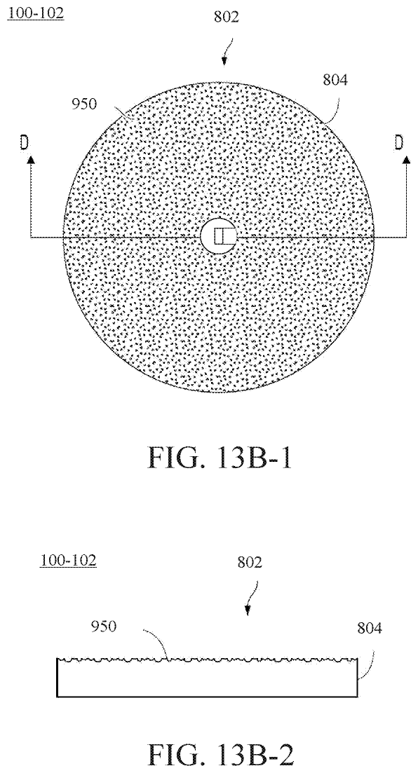

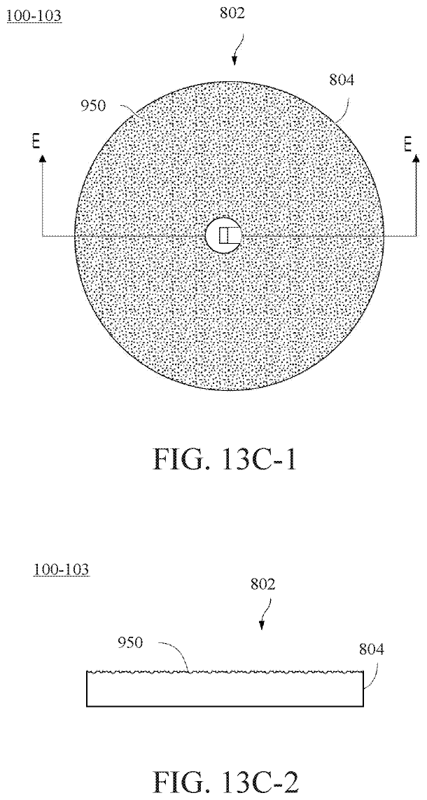

FIGS. 13A-1, 13B-1 and 13C-1 are bottom views showing OBDs 100-101 through 100-103 with the adhesive patch 106 removed (for the purposes of illustration) to show the exposed base surfaces 804 of each of OBDs 100-101 through 100-103. A non-directional (generally isotropic) surface texture 950 is applied to the exposed base surface 804 of the housing 802. The surface texture 950 can enhance adhesion of an adhesive patch 106 (not shown) to the base of the housing 802 by effectively increasing the surface area and providing mechanical interlocking features. Accordingly, the surface texture 950 may be provided on at least a portion of the base surface 804 comprising adhesive (not shown). The surface texture 950 may also facilitate the removal of moisture from the area adjacent the base surface 804 of the housing 802, by providing pathways to promote moisture egress, and by virtue of providing increased surface area for evaporation.

The surface texture 950 is a roughened surface as can be seen in the cross-sectional views of FIGS. 13A-2, 13B-2 and 13C-2 (which are zoomed in portions of cross-sections respectively along lines C-C, D-D and E-E of FIGS. 13A-1, 13B-1 and 13C-1 respectively). The roughened surface texture 950 may be applied to the base surface 804 of the housing with a bead blasting process. The surface texture may have an average roughness (Ra) of at least 0.35 μm. Preferably, the surface texture may have an average roughness (Ra) of between 0.35 μm and 2.24 μm. Preferably, the surface texture may have an average roughness (Ra) of between 0.56 μm and 0.80 μm. Preferably, the surface texture has an average roughness (Ra) of between 1.12 μm and 1.60 μm. Preferably, the surface texture has an average roughness (Ra) of around 1.5 μm.

As depicted in FIG. 13A-1 and 13A-2 the surface texture may be relatively coarse, with relatively deep surface indentations (creating the surface roughness), and with relatively large average pitch (average separation between indentations). Alternatively, as depicted in FIGS. 13B-1 and 13-B2, and 13C-1 and 13C-2, the surface texture may be more fine, with smaller undulations/depth of the indentations, and smaller separation between the undulations/texture portions. Alternatively, the depths of the indentations may be relatively small, but with relatively large average separation (pitch) between indentations, and vice versa. A relatively fine surface finish, such as around a VDI 15, VDI 18 or VDI 21 surface finish, has been found to maximise the time to delamination of the OBD 100 from the adhesive patch 106.

### On-Body Devices 100-104 and 100-105

Referring to FIGS. 14A-1 through 14B-2, OBDs 100-104 and 100-105 are shown according to an embodiment. OBDs 100-104 and 100-105 may be the same as OBD 100-1 of FIGS. 1B-1 to 1B-3, and may include the same features, except as explained below. The optional features and alternatives described in relation to OBD 100-1 may similarly be applied to OBDs 100-104 and 100-105 in a corresponding manner. In some examples, OBDs 100-104 and 100-105 may be similar to OBDs 100-80 through 100-104, and features described in relation to those embodiments may be applied to OBDs 100-104 and 100-105.

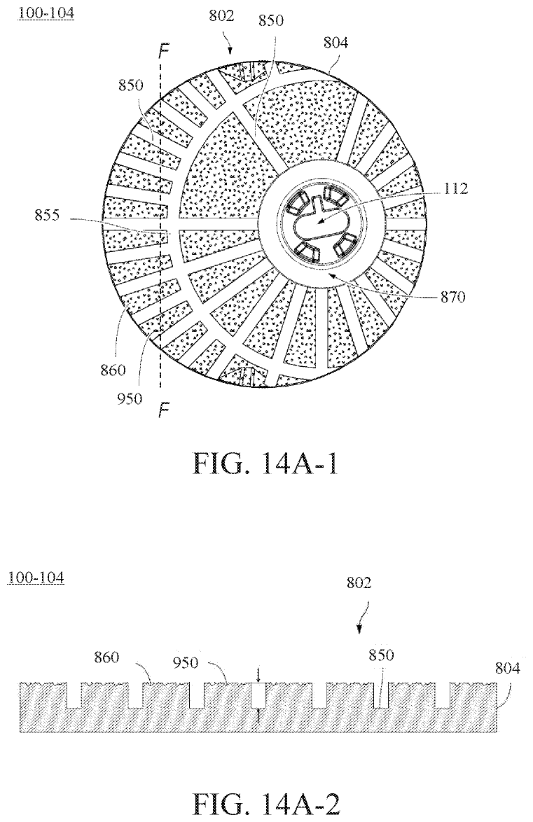

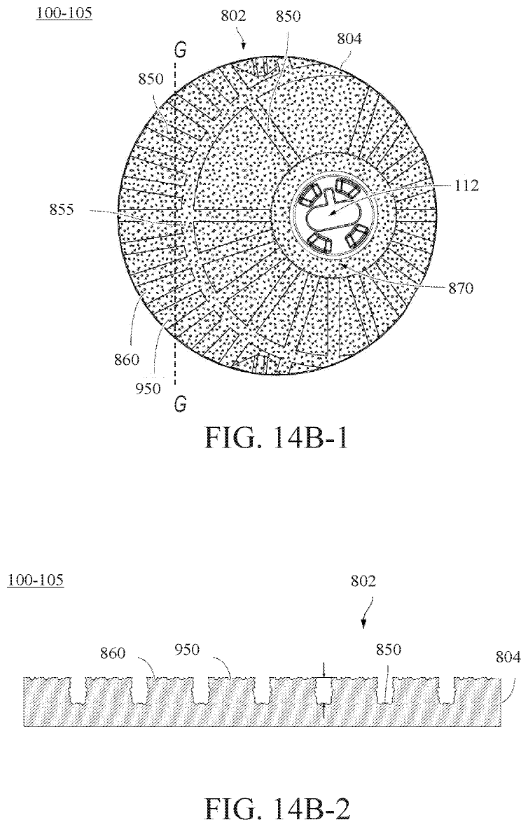

FIG. 14A-1 depicts a non-directional surface texture 950, such as the non-directional surface texture of FIGS. 13A-1 through 13C-2, as applied to the base surface of an OBD having channels 850, 855, such as in OBD 100-80. Accordingly, the non-directional surface texture 950 may have any of the features described herein in connection with FIGS. 13A-1 through 13C-2, including in respect of coarseness, depth, pitch, and variation across the surface. Similarly, the channels 850, 855 may have any of the features described herein in connection with FIGS. 8A-1 through 10 and 12A through 12G.

In the implementation of FIG. 14A-1, and as shown in FIG. 14A-2 as a zoomed in cross-section along line F-F of FIG. 14A-1, the surface texture 950 is provided only on the proximal portions 860 of the base surface 804 of OBD 100-104 (and not within the channels 850, 855). In this location, the surface texture 950 assists with adhesion to the adhesive patch 106 (not shown) which contacts the proximal portions 860. The surface texture 950 may also promote moisture removal by enhancing the surface area for evaporation, and by enabling distribution and routing of moisture across the base surface 804 of the housing 802.

In the implementation of FIG. 14B-1, and as shown in FIG. 14B-2 as a zoomed in cross-section along line G-G of FIG. 14B-1, the surface texture 950 is also provided within the channels 850, 855 of OBD 100-105. This surface texture 950 may be on the base and/or on the sidewalls of the channels 850, 855. This approach can assist with ease of manufacture, and can assist with adhesion of the adhesive patch 106, particularly when the channels 850, 855 are shallow such that the adhesive patch 106 can flex and contact a bottom thereof. The presence of surface texture 950 within the channels can also improve moisture egress by increasing the surface area of the channels, thereby promoting evaporation.

As shown in FIGS. 14A-2 and 14B-2, the depth of the channels 850, 855 is greater than the average depth of the surface texture 950. For instance, the average channel depth may be at least 10 μm greater than the surface texture 949 average depth, or preferably at least 50 μm greater, or more preferably at least 100 μm greater, or more preferably at least 150 μm greater.

### Removable Patch Portions

During testing, the inventors figured out that if an adhesive patch is adhered to a user's skin and the edge of the patch begins to stop adhering properly to the skin (e.g., peel), then the rest of the patch may cease to adhere surprisingly quickly after that. The inventors found this to be the case for several reasons. For instance, if the edge of the patch peels even slightly, the exposed adhesive may catch on nearby clothing, etc., which further acts to peel the patch away. Second, a properly adhered patch is generally difficult to remove as any forces pulling the patch off will be distributed evenly about the adhesive; however, this is not the case if an edge has started to peel as any forces applied to that edge will result in a stress concentration at the adhesive connecting the peeled edge to the skin.

The inventors have discovered that several approaches are effective at either minimising the likelihood of peel beginning or minimising the risk of the peel quickly progressing, or both.

The first approach is to provide an adhesive patch which comprises one or more removable patch portions releasably connected to the patch. These removable patch portions can be removed from, or detached from, the patch as desired by the user. The patch is configured such that the edges of the removable patch portions are likely to peel first. As will be explained further below, when the edge of a removable patch portion does peel, this removable patch portion can be removed thereby revealing or exposing a “fresh” or properly adhered edge; this may be described herein as “refreshing” the edge. Unlike the old peeling edge, the properly adhered edge should remain well adhered to the user's skin for a useful length of time. The removable patch portion can be releasably connected to the remaining patch in such a way that it is easy to remove the removable patch portion without causing the remaining patch to unstick from the skin. A plurality of removable patch portions may be provided so that the peeling areas of the patch edge can be precisely removed while minimising the amount of properly adhered edge that need be removed.

As outlined above, in some aspects of the present disclosure an adhesive patch (or adhesive patch assembly) may be provided with one or more removable patch portions. The patch is configured such that when an edge of the removable patch portion begins to peel, the removable patch portion can be removed to reveal a properly adhered edge; this may be termed “refreshing” the edge. When the outer edges of the adhesive patch are properly adhered to a user's body, the patch is likely to stay attached to the user's body for the desired period of time. In contrast, if any of the outer edges of the adhesive patch start to peel, the peeling may quickly progress until the adhesive patch is no longer properly secured to the user's body. Therefore, refreshing a peeled edge by removing a removable patch portion advantageously extends the wear duration of the adhesive patch.

It is envisaged that an adhesive patch assembly may be provided in least two different configurations, although many variations of these configurations exist, as outlined below. In a first configuration, the adhesive patch assembly will comprise and/or be associated with an on-body device; in this configuration, the adhesive patch assembly will be the primary means of securing the on-body device to the skin/the user's body. The adhesive patch of the first configuration (see e.g., adhesive patch 106) may be termed an on-body device patch. In a second configuration, the adhesive patch assembly will form an overpatch configured to be placed over an on-body device that is already secured to the skin to further secure the on body-device to the skin.

Where the adhesive patch assembly comprises an on-body device, an upper surface of the adhesive patch assembly may comprise a device adhesive which secures the on-body device to the adhesive patch assembly. This device adhesive may be located on the upper surface of a primary patch portion, as will be discussed further below. Other securing means can be used to secure the on-body device to the adhesive patch assembly. The primary patch portion may also comprise an aperture large enough to pass a sensor or cannula of the on-body device.

Where the adhesive patch assembly forms an overpatch, the adhesive patch assembly may comprise a hole or aperture through the primary patch portion which is large enough to pass over an on-body device that is already secured to a user's skin. The primary patch portion may also comprise a mating feature, for instance a rigid mating feature, which mates with the on-body device to minimise movement of the on-body device relative to the overpatch.

Both concepts are known and will not be discussed in further detail, but it suffices to say that any of the embodiments discussed below can be used as an adhesive patch assembly comprising an on-body device or as an overpatch by making the necessary modifications.

In this respect, there are a number of different ways in which an adhesive patch assembly (whether an on-body device patch or an overpatch) can be provided with these one or more removable patch portions.

Adhesive Patch with Removable Patch Portions Attached Proximate to, or to, Outer Periphery of Primary Patch Portion

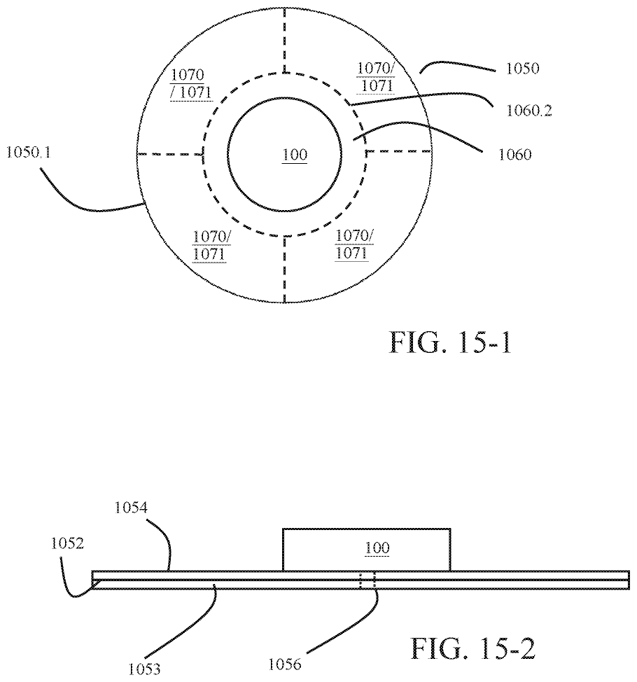

In one aspect of the disclosure, as shown in FIG. 15-1, an adhesive patch assembly 1050 comprises a primary patch portion 1060, a plurality of removable patch portions 1070, as well as an OBD 100. The primary patch portion and the plurality of removable patch portions (collectively “patch portions”) are, in this case, formed of a continuous layer of backing material (which is formed from a non-woven material, e.g. polyurethane or polyester spunlace). Before the adhesive patch is applied to a user's skin, the patch portions may collectively form a substantially single layer, which may be said to extend in a single plane or curved surface (not shown). Moreover, the patch portions of the present disclosure may be considered substantially two-dimensional, having a thickness that is much smaller than their length or width. The OBD 100 can be the same as any of the previously described OBDs (e.g. 100-1 to 100-105) or different.

As shown in FIG. 15-2, the layer 1051 of backing material defines a lower surface 1052 configured to face a user's skin or body when the assembly is in use and an upper surface 1054 opposite to the lower surface configured to face away from user's skin when the assembly is in use. The upper surface of the layer 1051 of backing material comprises a device adhesive (which comprises an acrylic-based pressure sensitive adhesive) adhering the OBD 100 to the upper surface. The layer 1051 of backing material comprises an aperture 1056 (shown with dashed lines) through which a sensor or cannula of the OBD can pass to thereby enter the user's body. The adhesive patch assembly defines an outer periphery 1050.1 which, in this case, is the same as the outer periphery defined by the plurality of patch portions. The lower surface of the backing material is coated in a layer of adhesive, e.g. has an adhesive layer 1053, (which is an acrylic-based pressure sensitive adhesive) which extends substantially up to the outer periphery of the adhesive patch assembly; the adhesive layer does not extend into or across the aperture in the layer of backing material.

In this instance, the OBD 100 is substantially circular and the adhesive patch assembly also defines a substantially circular outer periphery 1050.1. The outer periphery of the adhesive patch assembly does not need to have the same shape as the OBD but does in this case. The aperture 1056 is circular in this embodiment, but may have other shapes. The aperture is not shown again in further illustrations but can be present in any embodiment of the present disclosure; the aperture is not, however, essential as the sensor or cannula of the OBD may be able to penetrate the backing layer without need for the aperture.

As mentioned above, the adhesive patch assembly comprises a plurality of removable patch portions 1070. In this figure, there are four removable patch portions 1070 which are each releasably coupled to the primary patch portion 1060—specifically the outer periphery of the primary patch portion—as indicated by the dashed lines. Each of the four removable patch portions is substantially identically sized and shaped, although each oriented 90 degrees in respect of adjacent removable patch portions. The adhesive patch itself has a rotational symmetry of order 4, since any number of 90 degree rotations would result in the same illustration. As discussed above, if the edge of one of the removable patch portions begins to peel from the user's skin, a user may simply remove the removable patch portion in question thus revealing a “fresh” or non-peeling edge. By removing the removable patch portions in this manner, a user may significantly extend the wear duration of their on-body device.

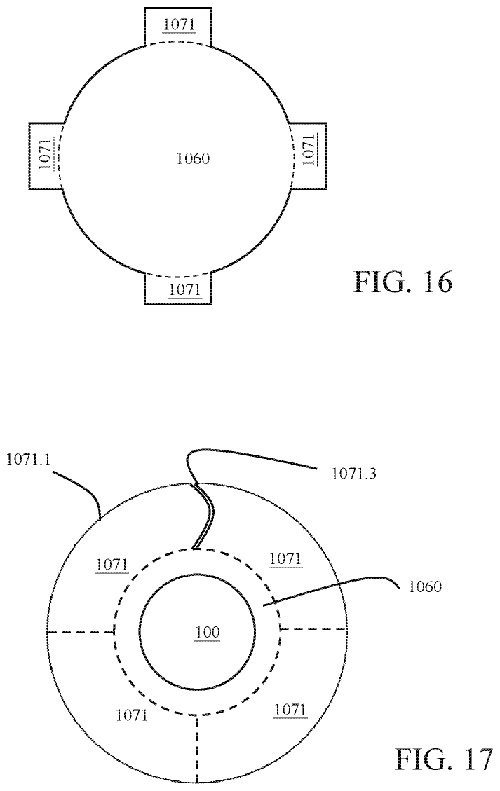

Within the context of this description, the removable patch portions shown in FIG. 15-1 are considered “first” removable patch portions 1071, since they are releasably coupled to the primary patch element. In FIG. 15-1, the “first” removable patch portions are releasably coupled to the primary patch element and each first removable patch portion is releasably coupled to another two first removable patch portions; however, in FIG. 16, the first removable patch portions 1071 are each releasably coupled to the primary patch portion 1060 only. No OBD is present in the embodiment of FIG. 16. In later described embodiments, the first removable patch portions may be removably attached to “non-first” removable patch portions.

In the embodiment shown in FIG. 15-1, the first removable patch portions 1071 have annular section shapes; collectively, the first removable patch portions form an annulus with a constant outer radius and a constant inner radius.

In the embodiment shown in FIG. 15-1, the first removable patch portions 1071 fully encompass the primary patch portion 1060 and there are no gaps between adjacent ones of the first removable patch portions 1071. By fully encompass, it is meant that any imaginary line (whether straight or curved) drawn outwardly (in the plane of the figure) from the primary patch portion will pass through at least one of the first removable patch portions and/or along a releasable coupling between two of the first primary patch portions. This is advantageous as it means that in all radial directions, an edge of a removable patch portion is likely to peel before the primary patch portion peels, such that the edge can easily be “refreshed”.

In other embodiments, such as shown in FIG. 17, the first removable patch portions may surround or encompass the primary patch portion in such a way that a) any straight line extending outwardly from the primary patch portion to the outer periphery 1071.1 of the first removable patch portions will pass through at least one first removable patch portion but b) a curved line extending outwardly from the primary patch portion may extend to the outer periphery 1071.1 of the first removable patch portions without passing through one of the first removable portions. This effect may occur when at least two of the first removable portions are separated by a curved gap extending from the outer periphery 1071.1 of the first removable portions to the outer periphery of the primary patch portion. In particular, the displacement of the curve (e.g. the maximum normal distance of the curve from a straight line joining the end-points of the curve) may be greater than the width of the gap, such that a straight line cannot extend from the outer periphery 1060.1 of the primary patch portion to the outer periphery 1071.1 of the first removable patch portions without contacting either first removable patch portion forming the gap. However, it is envisaged that this effect could be realised in other ways. Such a design is advantageous in that in most, or all, radial or outward directions, it is likely that an edge of a first removable patch portion 1071 will peel before an edge of the primary patch portion 1060 peels; moreover, because of the curved gap 1071.3 between the two first removable patch portions, these first removable patch portions can be removed more easily from one another.

As discussed above, by fully encompassing the primary patch portion and having no gaps between adjacent ones of the first removable patch portions, it is ensured that any peel that occurs at the outer periphery 1050.1 of the adhesive patch can be “refreshed” at least once by removing a removable patch portion. However, the first removable patch portions can also fully encompass the primary patch portions even if gaps exist between adjacent ones of the first removable patch portions. For instance, as shown in FIG. 18, gaps 1071.3 between each of the first removable patch portions may be formed by oval-shaped cut-outs; however, the first removable patch portions still fully encompass the primary patch portion. It is generally desirable for the first removable patch portions to be in contact with, or releasably coupled with, at least 30% of the outer periphery of the primary patch portion.

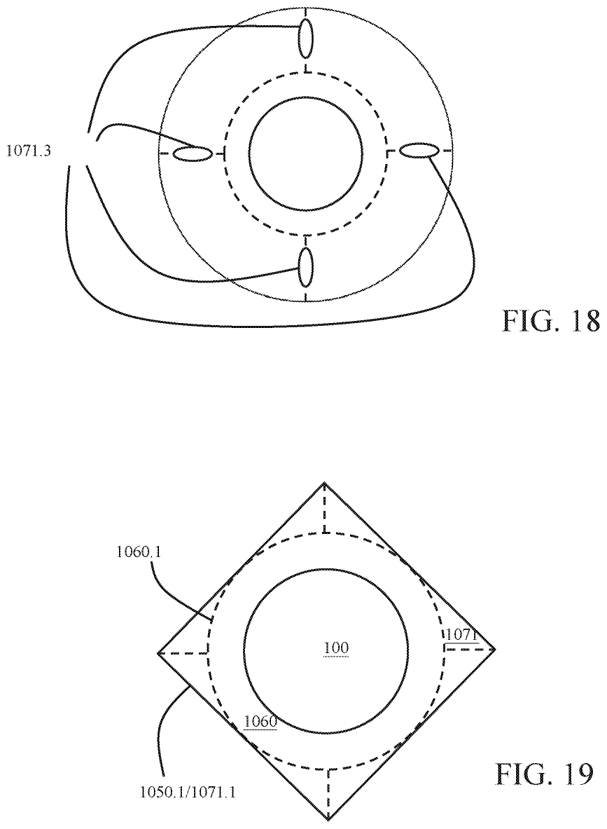

The same, or similar, benefit arises if the first removable patch portions define a continuous outer periphery 1071.1 which is, at all locations, more outward than an outer periphery 1060.1 defined by the primary patch portion, as in the embodiment of FIG. 15-1, for example. It is still advantageous, however, if the first removable patch portions define a) a discontinuous outer periphery or b) a continuous outer periphery 1071.1 of which some locations are no more outward than an outer periphery 1060.1 defined by the primary patch portion. This latter situation is shown in FIG. 19, wherein eight first removable portions 1071 (only one is labelled) are releasably coupled to a primary portion to form an adhesive patch having a substantially square outer periphery 1050.1. In this case, although the outer periphery 1071.1 of the first removable patch portions is continuous, it is also coincident with (e.g. no more outward than) the circular outer periphery 1060.1 of the primary patch portion at four locations. If peel starts to occur at these locations where the respective outer peripheries are coincident, removal of a first removable patch portion 1071 may be less effective than if the outer periphery 1071.1 of the first removable patch portions was more outward at all locations than the outer periphery 1060.1 of the primary patch portion.

In respect of FIG. 15-1 it has been stated that the first removable patch portions are releasably coupled to the outer periphery of the primary patch portion. However, they may also be releasably coupled proximate to the outer periphery of the primary patch portion, rather than to the outer periphery itself. For instance, this could mean that at least some, or all, of the first removable patch portions may be releasably coupled to respective locations of the primary patch portion inward of the outer periphery. In a more specific example, at least some, or all, of the first removable patch portions may be releasably coupled to the upper surface or the lower surface of the primary patch portion at respective locations of the primary patch portion inward of the outer periphery. In such situations, the primary patch portion may overlap with the respective first removable patch portions, such that the total thickness of the patch at that location is substantially equal to the thickness of the primary patch portion and the thickness of the respective first removable patch portion.

Adhesive Patch with “Second” and “Third” Removable Patch Portions

As discussed above, “non-first” removable patch portions may be provided. An example of such portions is shown in FIG. 20, wherein the adhesive patch comprises “second” 1072 and “third” 1073 sets of removable patch portions 1070. As shown, four second removable patch portions 1072 are releasably coupled to the respective four first removable patch portions 1071 but are not releasably coupled to the primary patch portion 1060. The four third removable patch portions 1073 are releasably coupled to neither the primary patch portion 1060 nor to any of the first removable patch portions 1071; instead, in this embodiment, the four third removable patch portions 1073 are releasably coupled to the respective four second removable patch portions 1072. No OBD is shown in this embodiment.

Here, “releasably coupled” means that the two named portions are directly connected by a releasable coupling; that is to say, the two named portions are directly adjacent one another and separated or joined by a releasable coupling (or a releasable coupling interface). Therefore, “not releasably coupled” means that the two named portions are not directly connected by a releasable coupling or releasable coupling interface. Two or more “not releasably coupled” portions may still be indirectly coupled or connected to one another via a series of releasable couplings, e.g. by means of a series of intervening removable patch portions. This is the case, e.g., for a third removable portion being ultimately coupled with the primary patch portion by the releasable coupling between the primary patch portion and the first removable patch portion, the releasable coupling between the first removable patch portion and the second removable patch portion, and finally the releasable coupling between the second removable patch portion and the third removable patch portion.

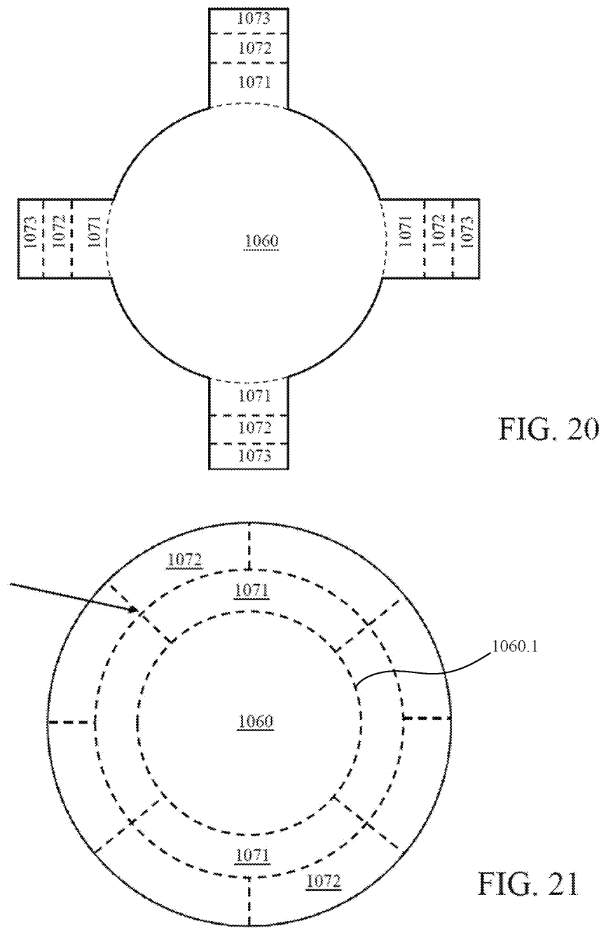

It is advantageous to make use of second and/or third removable patch portions, as doing so can provide additional opportunities to “refresh” the edge in any given radial direction. This is particularly true in cases where multiple sets of removable patch portions (e.g. the sets of first and second removable patch portions) substantially or fully encompass the primary patch portion. Such an embodiment is shown in FIG. 21, where the adhesive patch assembly comprises a primary patch portion 1060 having a substantially circular outer periphery 1060.1. Each of a set of four first removable patch portions 1071 have an annular section shape and are releasably coupled to the outer periphery 1060.1 of the primary patch portion 1060; together, the set of four first removable patch portions 1071 defines an annulus. A set of eight second removable patch portions 1072 is also provided; each of these also defines an annular section. It may be advantageous to provide a greater number of removable patch portions more outward of the primary patch element (e.g. second or third removable patch portions) relative to the number of removable patch portions less outward of the primary patch element (e.g. first removable patch portions). This is because, in most designs, the more outward the patch portions relative to the primary patch portion, the greater distance they must cover to encompass the primary patch portion. This is most obviously demonstrated in the case of a circle where the circumference increases linearly with radial distance. Moreover, given a larger outer periphery of the adhesive patch, it is more likely that a part of the outer periphery will start to peel; therefore, having a greater number of removable patch portions close to, or defining, the outer periphery allows for a more precise response to the peeling.

In the embodiment of FIG. 21, each of the removable patch portions is delineated by at least one releasable coupling or releasable coupling interface (which may also be termed “a line of weakness” or a “frangible boundary”) extending in an outward or radial direction relative to the primary patch portion 1060. As shown, a second removable patch portion 1072 may be at least partly delineated by an outwardly or radially extending releasable coupling that is aligned with an outwardly or radially extending releasable coupling that at least partly delineates a first removable patch portion 1071; one of four such pairs of aligned releasable couplings is indicated by the arrow in FIG. 21. As shown, the other four outwardly extending releasable couplings that at least partly delineate the second removable patch portions 1072 (directly North, East, South, and West in the Figure) are not aligned with any outwardly extending releasable couplings that at least partly delineate the first removable patch portions.

Speaking more generally, it is possible for two or more outwardly extending releasable couplings to be aligned along the particular outward direction, or it may be the case that an outwardly extending releasable coupling is not outwardly aligned with any other outwardly extending releasable coupling. It may also be the case that no outwardly extending releasable coupling aligns with another outwardly extending releasable coupling. It may be advantageous to use aligned outwardly extending releasable couplings since the manufacturing process may be simpler; however, it is also advantageous to use non-aligned outwardly extending releasable couplings since an unintentional tear or break in one outwardly extending releasable coupling is less likely to propagate along another outwardly extending releasable coupling if they are not aligned.

Speaking again with respect to FIG. 21, the removable patch portions 1071 and 1072 may be said to define a plurality of concentric rings or annuli, with a first concentric ring formed by the first removable patch portions 1071 and a second concentric ring formed by the second removable patch portions 1072. Although not shown, a third or more concentric rings could also be provided by the third removable patch portions 1073. As discussed, in this case there are four first removable patch portions and eight second removable patch portions; however, these quantities are not essential and any number of first removable patch portions could be provided together with any number of second removable patch portions (and optionally third removable patch portions). The first and second concentric rings have respective inner and outer peripheries each having a constant radius; however, the radius may alternatively vary with radial position. Any radial or outwardly extending releasable couplings that delineate removable patch portions in the first concentric ring may align with, or may be offset from, any respective radial or outwardly extending lines of weakness that delineate removable patch portions in the second concentric ring; this also applies to a third concentric ring, wherein any radial or outwardly extending releasable couplings that delineate removable patch portions in the second concentric ring may align with, or may be offset from, any respective radial or outwardly extending lines of weakness that delineate removable patch portions in the third concentric ring.

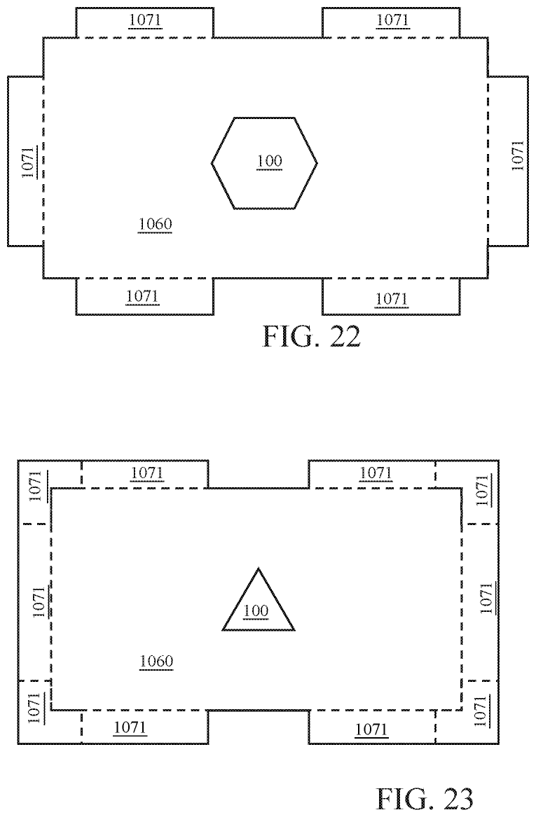

FIGS. 22 and 23 show adhesive patch assemblies having a plurality of different shapes. For instance, FIG. 22 shows a substantially rectangular primary patch portion 1060, substantially rectangular first removable patch portions 1071, and a hexagonal on-body device 100. FIG. 23 shows a substantially rectangular primary patch portion 1060, four substantially rectangular first removable patch portions 1071, four corner or L shaped first removable patch portions 1071, and a triangular on-body device 100. Many other combinations of shapes are possible.

Adhesive Patch with Path-Following Removable Patch Portions

The present disclosure envisages the use of more complex removable patch portions which follow a path as they are removed; these patch portions may be termed path-following or self-guided removable patch portions.

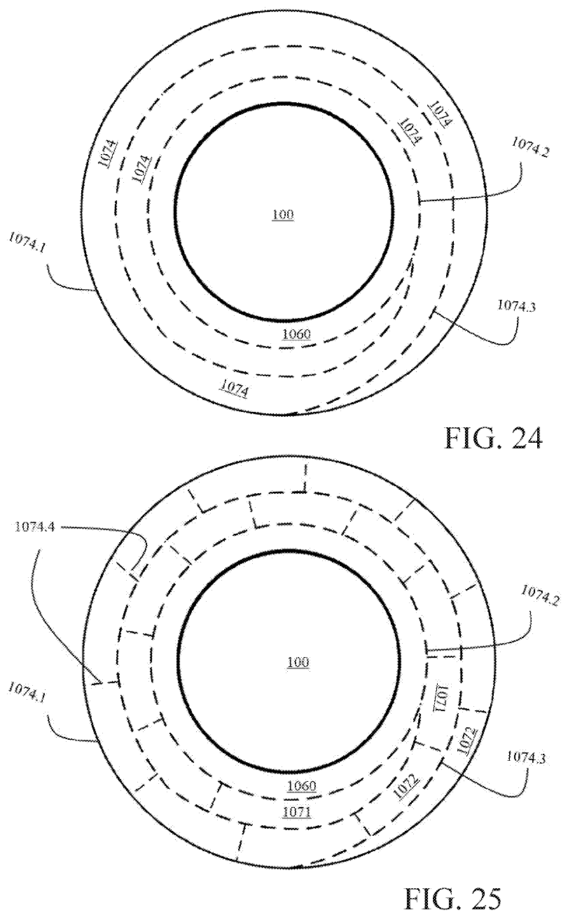

One embodiment in which a path-following or self-guided removable patch portion is used is shown in FIG. 24. As shown, a primary patch portion 1060 having a substantially circular outer periphery is surrounded by a path-following removable patch portion 1074 which extends in a spiral pattern away from the outer periphery of the primary patch portion. As can be seen, the path-following removable patch portion 1074 defines an inner periphery 1074.2 and an outer periphery 1074.1, wherein the inner periphery is entirely in contact with the outer periphery of the primary patch portion. A guiding releasable coupling or guiding releasable coupling interface 1074.3 (e.g. a line of weakness or frangible boundary) extends in a spiral shape from the outer periphery 1074.1 of the path-following removable patch portion to the inner periphery 1074.2 of the path-following removable patch portion; in doing so, the guiding releasable coupling releasably couples the path-following removable patch portion to itself. The guiding releasable coupling may therefore be termed a releasable self-coupling. The releasable coupling extends to meet both the inner periphery 1074.2 of the path-following removable patch portion and the outer periphery 1074.1 of the path-following removable patch portion non-orthogonally, e.g., not a right angle. In other embodiments, not shown, the guiding releasable coupling may meet one of, or both, the inner periphery 1074.2 of the path-following removable patch portion and the outer periphery 1074.1 of the path-following removable patch portion orthogonally, e.g. at a right angle.

In other words, the path-following removable patch portion 1074 comprises a continuous annulus which has been provided with a releasable coupling or line of weakness extending from the outer periphery of the annulus to the inner periphery of the annulus, so that the guiding releasable coupling or line of weakness couples the path-following removable patch portion to itself. More generally, the shape of the path-following removable patch portion 1074 may follow a closed line or curve, such that the path-following removable patch portion 1074 has an inner and outer periphery offset from the closed line or curve, wherein one or more lines of weakness extend from the inner to the outer periphery of the path-following removable patch portion and thereby across the closed line.

When removing the path-following removable patch portion it will preferentially follow its path when removed in the correct predetermined manner. In this case, the path-following removable patch portion should be removed in the vicinity of where the guiding releasable coupling meets the outer periphery, such that the guiding releasable coupling immediately starts to break and thereby causes the path-following removable patch portion to follow a spiral removal path. While not shown, a marker or indicia may be used to show the correct point at which to begin the removal of the path-following removable patch portion. For instance, in this case, an indicia would be positioned at the point where the guiding releasable coupling or line of weakness meets the outer periphery of the path-following removable patch portion.

The spiral pattern of the guiding releasable coupling at least partly defines the path of removal of the path-following removable patch portion. As shown in FIG. 24, the path-following removable patch portion may extend in a spiral pattern having over two whole revolutions. However, it may also extend only a single revolution or it may extend three or more revolutions.

The guiding releasable coupling is shown to extend as a curved spiral; however, the guiding releasable coupling may extend as a square or rectangular spiral without any curved sides. Alternatively, the releasable coupling may extend having a mix of square and curved sides.

In the embodiment of FIG. 24, the path-following removable patch portion 1074 may be considered to comprise a single first removable patch portion 1071, since the path-following removable patch portion is releasably coupled to the primary patch portion 1060. In this embodiment, if a user wished to remove part of the path-following removable patch portion they may be able to tear the path-following removable patch portion along the width of the path, even if the absence of a releasable coupling, provided the material allows for this. Alternatively or in addition, the user may employ a tool, e.g., scissors, to cut the path-following removable patch portion along the width of its path.

In instances where there are a specified quantity of first removable patch portions, the path-following removable patch portion may be included within the specified quantity of first removable patch portions or it may be provided in addition to the specified quantity of first removable patch portions.

A similar embodiment is shown in FIG. 25, which is substantially identical to that shown in FIG. 24 except for the use of transverse releasable couplings 1074.4 (only two are labelled) along the width of the path of the path-following removable patch portion. These transverse releasable couplings delineate the path-following removable patch portion into a plurality of removable patch portions; these lines may be torn or broken to remove a removable patch portion from the path-following removable patch portion and therefore from the adhesive patch. The transverse releasable couplings may extend substantially orthogonally to one or more edges of the path at that location.

If the path-following removable patch portion is peeled along the direction of the path, the guiding releasable coupling defining the path will preferentially break; on the other hand, a peeling action or tearing action across the path may cause a transverse releasable coupling extending across the path to break instead.

The guiding releasable coupling may be weaker than the transverse releasable couplings to further encourage the path-following removable patch portion to follow the path; however, this is not essential as the direction in which the user applies a removal force will typically dictate whether the guiding releasable coupling breaks/continues to break or whether a transverse releasable coupling breaks.

The path-following removable patch portion of this embodiment may be said to be formed of a plurality of removable patch portions; for instance, there are nine removable patch portions which are releasably coupled to the outer periphery of the primary patch portion and thus nine first removable patch portions 1071. The remaining eleven removable patch portions are each releasably coupled to a respective one or more of the first removable patch portions and thus define eleven second removable patch portions 1072. The first and last of the first and second removable patch portions along the path are labelled. Although not shown, the path-following removable patch portion may comprise additional third removable patch portions which are releasably coupled to neither the primary patch portion nor the first removable patch portions.

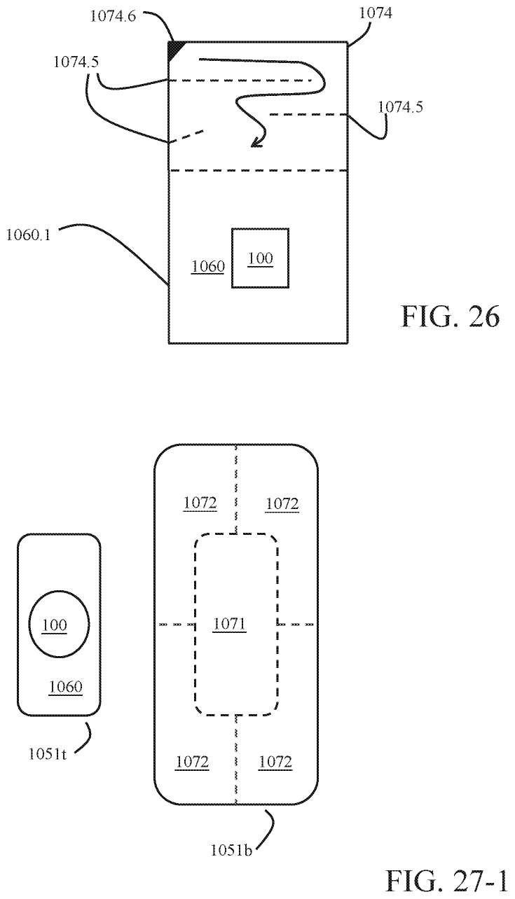

A different form of path-following removable patch portion is shown in FIG. 26, where the path-following removable patch portion 1074 is releasably coupled to one edge of the substantially square outer periphery 1060.1 of a primary patch portion 1060. Only one edge of the primary patch portion is releasably coupled to another removable patch portion in this embodiment, but in other embodiments at least some or all of the other edges may be coupled to other removable patch portions (including other path-following removable patch portions).

In this instance, the path-following removable patch portion 1074 is also substantially square or rectangular shaped and does not define an inner periphery contrary to the path-following removable patch portion discussed above. Here, the path of the path-following removable patch portion 1074 is dictated by guiding releasable couplings 1074.5 (or lines of weakness) extending from the edge of the path-following removable patch portion to respective points within the path-following removable patch portion; no guiding releasable couplings or set of guiding releasable couplings extends fully though the path-following removable patch portion (e.g. from one edge of the path-following removable patch portion to another). Therefore, these guiding releasable couplings may therefore be termed partial releasable couplings or partial lines of weakness. As before, these releasable couplings couple the path-following removable patch portion to itself and may therefore be termed “releasable self-couplings”. When the path-following removable patch portion 1074 begins to be removed from an edge proximate to the start of the path, the partial lines of weakness 1074.5 may sequentially or simultaneously break as the removal progresses, thereby guiding the path-following removable patch portion along its path. The path is shown by an arrow in this instance. The adhesive patch comprises a marker or indicia 1074.6 to indicate the correct location to begin removal of the path-following removable patch portion.

The locations, directions, and lengths of the guiding releasable couplings 1074.6 may be varied to dictate the path that is followed. For instance, a single guiding releasable coupling 1074.6 extending from an edge may cause the path to double back on itself as shown in FIG. 26; the use of another partial lines of weakness extending from a different edge, as also shown in FIG. 26, may cause the path to double back on itself again. A series of overlapping but offset guiding releasable couplings extending from alternating opposite edges of the path-following removable patch portion may cause the path to extend in a concertinaed manner, wherein the path repeatedly doubles back on itself. These guiding releasable couplings may extend orthogonally or non-orthogonally from the edge of the path-following removable patch portion; depending on the shape of the path-following removable patch portion, use of orthogonal or non-orthogonal guiding releasable couplings may help keep the path a substantially constant width.

### On-Body Device

The above discussed embodiments all include removable patch portions that are releasably coupled proximate to, or to, the outer periphery of the primary patch portion. However, this is not essential to the present disclosure. Instead, or in addition, removable patch portions could be releasably coupled to the lower surface of then primary patch portion, for instance to the adhesive layer of the primary patch portion. In such embodiments, the adhesive patch may have two or more parallel layers of backing material, each of these layers having an adhesive layer. In other words, the adhesive patch may comprises a top backing layer which is, in use, furthest away from the user's body and a bottom backing layer which, in use, is closest to (e.g. contacts) the user's body; the adhesive patch may additionally comprise intermediate backing layers between the top and the bottom backing layer, each of the intermediate backing layers having a respective adhesive layer.

In use, the adhesive patch may be secured to the user's body using the adhesive layer of the bottom backing layer; when an outer edge of the bottom backing layer starts to adhere poorly to the user's skin (e.g. in the manner outlined above), part or all of the bottom backing layer can be removed and the adhesive patch re-adhered to the skin using the revealed part(s) of the next backing layer (e.g. the top backing layer if there were only two backing layers to start, or otherwise one of the intermediate backing layers if there were more than two backing layers to start). The top backing layer may comprise or consist of a primary patch portion, as outlined above. The bottom backing layer (and any intermediate layers, if present) may be formed of removable patch portions, as outlined above. This means that a removable patch portion can be removed from the lowest backing layer to reveal part of the next lowest backing layer, which revealed part can then be used to re-adhere the adhesive patch.

In these embodiments, the top backing layer may be constructed in any of the ways outlined above, including with the use of the first removable patch portions releasably coupled proximate to, or to, the outer periphery of the primary patch portion and with the further use of second and third removable patch portions. Alternatively, the top backing layer may comprise a primary patch portion without any further removable patch portions.

The backing layer immediately under the top backing layer (the “top⁻¹” backing layer) may be constructed in the same manner as the top backing layer, except for the absence of the primary patch portion. The lack of a primary patch portions means that the first, second, and third removable patch portions are defined slightly differently in respect of the “top⁻¹” backing layer. In particular, the first removable portions of the “top⁻¹” backing layer are defined as those releasably coupled (e.g. adhered) to the adhesive surface of the primary patch portion of the top backing layer; the second removable patch portions of the “top⁻¹” backing layer are defined as those releasably coupled to the first removable patch portions but not releasably coupled to the adhesive surface of the primary patch portion of the top backing layer, and the third removable patch portions of the “top⁻¹” backing layer are defined as those that are not releasably coupled to either the primary patch portion of the top backing layer nor to the first removable portions of the “top⁻¹” backing layer. If there are only two backing layers, then the “top⁻¹” is the same as the bottom backing layer.

Any backing layers under the “top⁻¹” backing layer (e.g. “topⁿ” backing layers) can have removable patch portions defined in substantially the same way as the “top⁻¹” backing layer. In particular, the primary patch portion of the top backing layer may be projected along an axis (e.g. a vertical axis) extending from the top backing layer to the bottom backing layer; any removable patch portions of the “topⁿ” backing layer lying within this projected primary patch portion can be termed the first removable patch portions of the “topⁿ” backing layer, any removable patch portions of “topⁿ” backing layer lying outside the projected primary patch portion but releasably coupled to the first removable patch portions of the “topⁿ” backing layer can be termed the second removable patch portions of the “topⁿ” backing layer, and any removable patch portions of the “topⁿ” backing layer lying outside the projected primary patch portion and not releasably coupled to the first removable patch portions of the “topⁿ” backing layer can be termed the third removable patch portions of the “topⁿ” backing layer. If there are three or more backing layers, then the bottom backing layer is a “topⁿ” backing layer.

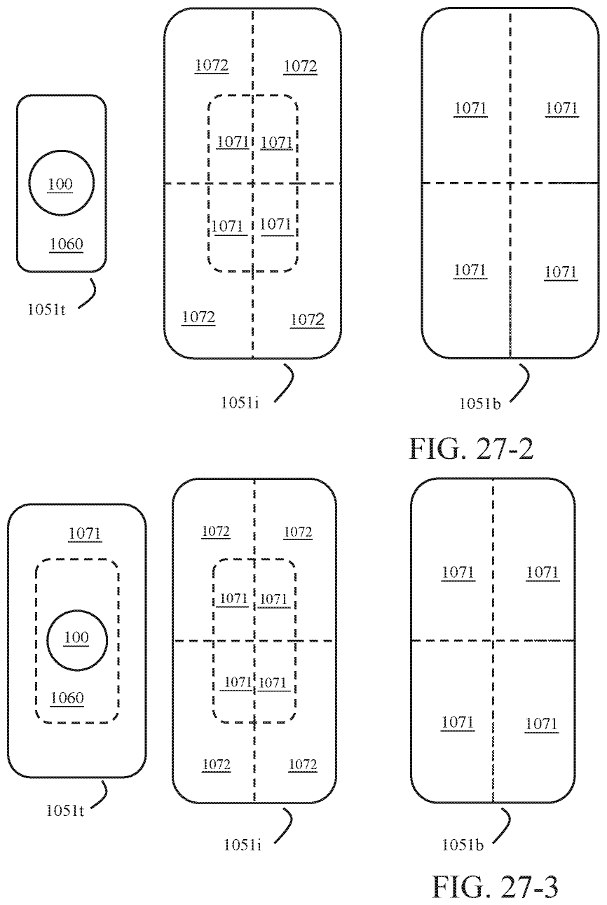

An embodiment according to this aspect of the present disclosure is illustrated in FIG. 27-1. As shown, an adhesive patch assembly comprises a top backing layer 1051t comprising a primary patch portion 1060 and accommodating a circular OBD 100. The primary patch portion 1060 defines the outer periphery of the top backing layer as having a rounded rectangular shape. The primary patch portion comprises an adhesive layer on its lower side (not shown). The adhesive patch assembly comprises a bottom backing layer 1051b, which is shown separately from the top backing layer 1051t for clarity. The bottom backing layer defines a similar rounded rectangular shaped outer periphery. The bottom backing layer comprises a first removable patch portion 1071 which is identical in shape and position to the outer periphery of the top backing layer 1051t; the first removable patch portion is releasably coupled to the adhesive layer of the primary patch portion of the top backing layer. The bottom backing layer further comprises four second removable patch portions 1072 which are each releasably coupled to an outer periphery of the first removable patch portion.

FIG. 27-2 shows an embodiment similar to that of FIG. 27-1; however, the adhesive patch assembly now comprises an intermediate backing layer 1051i. This intermediate backing layer is similar to the bottom backing layer of FIG. 27-1 except that it comprises four first removable patch portions in place of the first removable patch portion of FIG. 27-1. The bottom backing layer 1051b defines the same outer periphery as the intermediate backing layer 1051i; the bottom backing layer comprises four first removable patch portions 1071 which are releasably coupled to the respective adhesive layers of the first 1071 and second 1072 removable patch portions of the intermediate backing layer 1051i.

A further embodiment is shown at FIG. 27-3, which is substantially identical to that of FIG. 27-2, except that the top backing layer 1051t comprises a first removable patch portion 1071 which surrounds the primary patch portion 1060.

The embodiments shown in FIGS. 27-1 to 27-3 are for illustrative purposes only. An adhesive patch may comprise any number of backing layers, each of which can define any or the same outer peripheries, and each of which can comprise any number of first, second, and/or third removable patch portions, and each of which can be formed of any material (for instance, as defined under the heading “Shapes, Numbers, Materials”). The first, second, and/or third removable patch portions can be any shape (for instance, as defined under the heading “Shapes, Numbers, Materials”). The OBD can also be of any type and any shape (for instance, as defined under the heading “Shapes, Numbers, Materials”).

### Shapes, Numbers, Materials

So far, the illustrated primary patch portions have had substantially circular outer peripheries. However, this is not essential and many other shapes are possible. For instance, as shown in FIG. 22, the primary patch portion has a rectangular outer periphery, with the first removable patch portions 1071 also having a rectangular shape. The OBD 100 has a hexagonal shape.

The outer periphery of the primary patch portion does not have to be the same shape as the first removable patch portions, nor do each of the first removable patch portions have to be the same shape as each other. For instance, in FIG. 23, four of the first removable patch portions 1071 are rectangular, while the remaining four first removable patch portions are substantially corner shaped or L shaped. The OBD 100 is triangle shaped.

In general, the primary patch portion 1060 may define an outer periphery having a shape comprising one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The primary patch portions may have other shapes as well.

Similarly, the removable patch portions may each have a shape comprising one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The removable patch portions may have other shapes as well.

The outer periphery of the adhesive patch may also define an outer periphery having a shape comprising one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The outer periphery of the adhesive patch may have other shapes as well.

The on-body device may have a shape comprising one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The on-body device may have other shapes as well.

The aperture may also have a shape comprising one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The aperture may have other shapes as well.

Where an adhesive patch comprises multiple layers of backing material, each layer of backing material may define outer peripheries having the same shape or different shapes. The top backing layer may define an outer periphery that is smaller in area and/or more inwards at at least one location than the outer periphery defined by any other backing layer.

In particular, the top backing layer may define an outer periphery having a shape comprising one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The top backing layer may have other shapes as well.

The bottom and/or intermediate backing layers may define respective outer peripheries having a shape comprising one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The bottom and/or intermediate backing layers may have other shapes as well.

The primary patch portion may have a maximum dimension in the plane of the patch of 100 mm, 65 mm, 25 mm, or 20 mm. the primary patch portion may have a minimum dimension in the plane of the patch of at least 50% of the maximum dimension of the primary patch portion Outer periphery 1050.1 of the adhesive patch may have a maximum dimension in the plane of the patch of 100 mm, 65 mm, 25 mm, or 20 mm. The outer periphery 1050.1 of the adhesive patch may have a minimum dimension in the plane of the patch of at least 50% of the maximum dimension of the outer periphery 1050.1.

Where the adhesive patch comprises multiple layers of backing material, the top backing layer 1051t may have a maximum dimension in the plane of the patch of 100 mm, 65 mm, 25 mm, or 20 mm. The bottom 1051b and/or intermediate 1051i backing layers may have a maximum dimension in the plane of the patch of 100 mm, 65 mm, 25 mm, or 20 mm.

For completeness, it is noted that the outer periphery 1050.1 of the adhesive patch 1050 may be defined by the most outward edges of the patch portions that the adhesive patch comprises, e.g., the outer periphery 1050.1 of the adhesive patch 1050 may be defined by the most outward edges of the outer periphery 1060.1 of the primary patch portion, the outer periphery 1071.1 of the first removable patch portions 1071, and the outer peripheries (not labelled) of any of the second and/or third removable patch portions 1072 and 1073. Similarly, in embodiments having multiple layers of backing material, the outer periphery of the adhesive patch may be defined by the most outward edges of the set of backing layers. An example is shown in FIG. 27-1 where the outer periphery of the top backing layer 1051t is entirely inward of the outer periphery of the bottom backing layer 1051b; in this instance, the outer periphery of the adhesive patch would be identical to the outer periphery of the bottom backing layer 1051b.

Generally speaking, the adhesive patch may comprise or consist of one, two, three, four, or five first removable patch portions. Similarly, the adhesive patch may comprise or consist of one, two, three, four, or five second removable patch portions. Also similarly, the adhesive patch may comprise or consist of one, two, three, four, or five third removable patch portions. The adhesive patch may comprise one or more path-following removable patch portions; the one or more path-following removable patch portions may be defined by a plurality of removable patch portions. Where a number of first, second, and/or third removable patch portions has been specified, the one or more removable patch portions contributed by the one or more path-following removable patch portions may be included within this count or may be provided in addition to this count.

Where the adhesive patch comprises multiple layers of backing material, each layer may comprise the same number of removable patch portions or may comprise a different number of removable patch portions. Each layer of backing material may comprise the number of first, second, third and/o path-following removable patch portions specified above.

Generally speaking, the backing layer or backing layers used to implement the present disclosure may comprise a non-woven plastic fiber matrix (e.g. a non-woven material); this non-woven material may be a PET or polyurethane fiber matrix, more specifically a polyester or polyurethane spunlace non-woven material. The adhesive layer may comprise a pressure-sensitive adhesive, for instance an acrylic-based pressure-sensitive adhesive. The thickness of the backing layer may be between 10 μm and 300 μm, between 15 μm and 275 μm, between 40 μm and 200 μm, or between 50 μm and 150 μm. The thickness of the adhesive layer may be between 50 μm to 125 μm or 75 μm to 100 μm. Where the adhesive patch comprises at least one first and one second removable patch portions, the second removable patch portion may be formed of a more stretchy or more elastic material than the first removable patch portion. The inventors have figured out that more radially outward sections of the patch experience more strain than more radially inward sections of the patch; as such, making the second removable patch portion(s) more stretchy or more elastic than the first removable patch portion(s) may be beneficial. Similarly, the first removable patch portion(s) may be made from a more stretchy or more elastic material than the primary patch portion.

### Ease of Removal

In many of the above embodiments, releasable coupling interfaces (e.g. releasable couplings or lines of weakness) extend to the outer periphery of the adhesive patch. Because these releasable coupling interfaces extend to the outer periphery of the adhesive patch, they allow a user to easily access the releasable coupling interface to start breaking it, thus facilitating removal of a given removable patch portion. It can be helpful for these releasable coupling interfaces to extend to the outer periphery 1050.1 of the adhesive patch 1050 in an orthogonal manner in the immediate vicinity of the outer periphery 1050.1; this allows the releasable coupling interface to be broken easily by the user but minimises the risk that the releasable coupling interface starts to break unintentionally (e.g. from contact in a tangential direction to the outer periphery). However, away from the outer periphery it may be helpful for the same releasable coupling interface to extend tangentially to the outer periphery 1050.1; this can allow the removable patch portion 1070 to be peeled off in a direction that minimises the risk of peeling away the remainder of the adhesive patch 1050. The adhesive patch of FIG. 15 is constructed in such a fashion, since once one of the outwardly extending releasable coupling interfaces is broken, the removal of a given removable patch portion 1070 is then facilitated by the breaking of the circumferential releasable coupling interface between the primary patch portion 1060 and the given removable patch portion 1070. This can similarly be seen in FIG. 21, wherein four of the releasable couplings extend radially or inwardly from the outer periphery of the adhesive patch and then circumferentially around the outer periphery defined by the first removable portions.

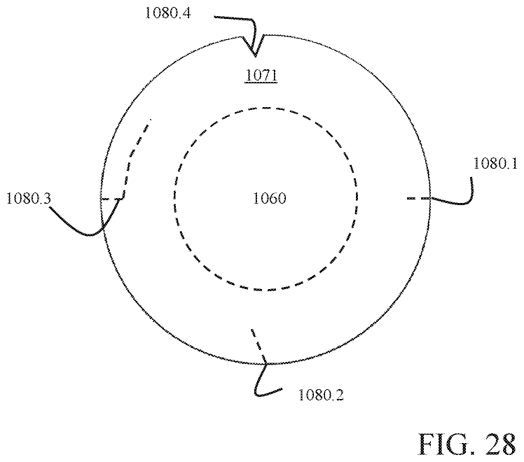

It can also be helpful to provide releasable coupling interfaces (e.g. releasable couplings or lines of weakness) that extend only partially into a given removable patch portion. This may allow a given removable patch portion to be removed more easily. This is particularly the case if the removable patch portion forms a single continuous ring or loop with no defined end. As shown in FIG. 28, a single removable first patch portion 1071 may entirely encompass the primary patch portion 1060. In this case, a line of weakness 1080.1 partially extends into the removable patch portion orthogonally to the local edge of the removable patch portion and a line of weakness 1080.2 partially extends into the removable patch portion non-orthogonally. One or more lines of weakness 1080.3 extend in a first direction from the local edge of the removable patch portion and then extend in a second direction different from the first. Finally, a notch 1080.4 extends into removable patch portion 1071; a notch represents a discontinuous area or an area of material removal which does not include a releasable coupling. The notch 1080.4 also serves to cause the removable patch portion 1071 to break more easily.

Generally speaking, any given removable patch portion may comprise a releasable coupling interface (e.g. a releasable coupling or line of weakness) extending orthogonally or non-orthogonally from an edge of the removable patch portion to a point within the removable patch portion (that is to say, only partially within the removable patch portion). This releasable coupling or line of weakness may extend in a different direction away from the edge in question. A removable patch portion may alternatively or additional comprise a notch in one or more of its edges.

As another approach to improving the ease at which a removable patch portion may be removed, any given removable patch portion may be provided with a pull tab. The pull tab may not comprise an adhesive layer on its lower surface and may therefore not adhere to the user's skin. The pull tab may extend from the edge of the removable patch portion (e.g. it may extend in substantially the same plane as the removable patch portion) or from the upper surface of the removable patch portion.

### Releasable Coupling and Adhesive Layers

The terms “releasably coupled” and “releasable coupling interface” have been used frequently herein. These terms refer to a coupling or coupled interface between two portions or between two parts of the same portion which is intended to break (e.g. which preferentially breaks) as the two portions or two parts of the same portion are separated. These releasable coupling interfaces, when provided between two different portions, allows for easier separation of these two portions; more generally, the releasable couplings allow for removal of at least one, if not both, of the releasably coupled portions from the adhesive patch independently of the remaining portions of the adhesive patch.

A portion of a patch may be considered a removable patch portion if the patch is constructed or configured in a manner where that portion can be removed substantially independently of the remaining patch, and/or if the adhesive patch comprises structural features which make it easier to remove that specific portion of the patch. Herein, “removed substantially independently of the remaining patch” may mean that the portion can be removed without causing the remaining edges of the patch to peel or that any peel that does occur with respect to the remaining edges of the patch is less than if the portion were not releasably coupled.

It may be that a user intends to break a releasable coupling to separate two portions. Alternatively, or in addition, it may be the case that in the intentional removal of a portion, one or more releasable couplings break, even if not necessarily intended by the user; this is more likely the case in respect of path-following removable patch portions, which follow a path if the user removes them starting from a predetermined point.

There are many ways to releasably couple two portions or two parts of the same portion. In particular, such releasable couplings may be formed by shared lines of weakness or shared frangible boundaries created or provided between two portions or two parts of the same portion. For instance, lines of weakness may be formed by perforations extending through a continuous layer of material defining two or more of the removable patch portions. As the line of perforations is weak relative to the surrounding material it preferentially breaks when an adjacent removable patch portion is removed. In this sense, the lines of weakness may act to delineate two or more portions from a single continuous piece of material. As described later, the lines of weakness may be formed in other ways and, similarly, the patch portions may be formed from discontinuous pieces rather than a continuous piece of material.

In another approach which utilises a continuous layer of material, two portions or two parts of the same portion may be releasably coupled by a local thinning of the layer of material, wherein the thinned material is configured to break. For instance, a releasable coupling or line of weakness may be thinner along its length than the material of the continuous layer of material immediately either side of the thinning. The thinning may be formed by material removal from the upper surface of the continuous layer of the material, the lower surface of the continuous layer of material or both. The thinning may be uniform along the length of the releasable coupling or line of weakness or it may vary. The thinning may also be intermittent along the length of the releasable coupling or line of weakness.

In other approach, two patch portions may be formed from respective separate or discontinuous pieces of material (which may have originally been a continuous piece or layer of material until the portions were formed). In one example, these portions may be releasably coupled (e.g. releasable coupling or line of weakness may be formed) by using part of the adhesive layer of one portion to adhere to part of the upper surface of the other portion to thereby releasably adhere the portions together. When viewed in a plan view (e.g. along the thickness direction of the patch), one portion would overlap the other. One portion may adhere to the upper surface of the other along the whole length of the releasable coupling or along only a portion of the length of the releasable coupling; alternatively, the portions may alternatively adhere to the others upper surface along the length of the releasable coupling. In another example using discontinuous pieces of material, the portions may be releasably coupled edge to edge by an additional adhesive strip which lies between the releasably coupled edges; in a plan view, the portions would not overlap but would instead be separated by the additional adhesive strip. The adhesive strip could be formed of the same adhesive as any of the adhesive layers.

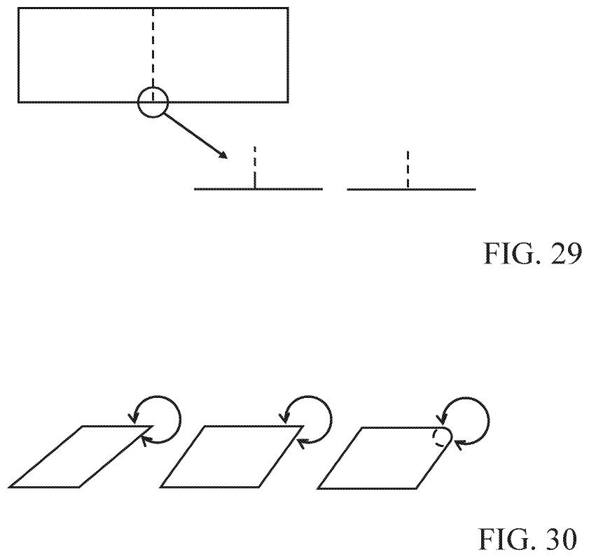

A given releasable coupling does not have to be uniform along its length and can comprise lengths at which the releasably coupled portions are, in fact, on a local scale not releasably coupled. This can be seen as similar to the nature of perforations which provide alternating regions where the portions are non-releasably coupled and regions where the portions are not coupled at all. Therefore, a releasable coupling may render two portions easier to separate, even if at some locations along the length of the releasable coupling the two portions are in fact non-releasably coupled. For instance, in the case where it is stated that a releasable coupling extends to or from an edge, at a local scale, the two portions or two parts of the portion could be non-releasably coupled at that edge, provided the remainder of the releasable coupling renders it easier for that non-releasable coupling to break. Alternatively, where it is stated that a releasable coupling extends to or from an edge, at a local level, the two portions or two parts of the portion may also be releasably coupled at that edge. FIG. 29 illustrates this concept by providing two enlargements of a releasable coupling extending to an edge; on the left, the edge remains non-releasably coupled at a local level, while on the right the edge is also releasably coupled.

As discussed, the patch portions typically comprise adhesive layers. For instance, each of the primary patch portion and removable patch portions directly or indirectly connected to it (e.g. first, second, and third removable patch portions) may comprise adhesive layers. These adhesive layers may form a substantially continuous layer. Alternatively, these adhesive layers may form a substantially discontinuous layer of adhesive. In another aspect, each of the removable patch portions may comprise adhesive layers, which layers may form a substantially continuous layer. Alternatively, these adhesive layers may form a substantially discontinuous layer of adhesive. Generally speaking, the adhesive layer may substantially extend to the outer periphery of the adhesive patch. Where the adhesive patch comprises multiple layers of backing material, each of these may comprise adhesive layers which substantially extend to the respective outer peripheries of each of the layers of backing material.

Regardless of whether the adhesive patch or each of the multiple layers of backing material comprises a substantially continuous or discontinuous adhesive layer, it may be advantageous for the adhesive layer to comprise less or no adhesive along the length of the releasable coupling or line of weakness. By applying less or no adhesive along the length of the releasable coupling, the releasably coupled portion becomes easier to remove from the patch and is also less likely to cause the remaining patch to peel as it is removed (since there is less adhesive which could act to couple the removed portion to the remaining patch). Alternatively, the adhesive layer or layers may comprise a weaker adhesive along the length of a releasable coupling interface (also referred to as “releasable coupling”). The adhesive layer of the adhesive patch or one or more of the layers of backing material may therefore comprise no adhesive along at least one or all releasable coupling interfaces, less adhesive along at least one or all releasable coupling interfaces, or a weaker adhesive along at least one or all releasable coupling interfaces.

In FIG. 30 a removable patch portion having a rhombus shape has two sharp corners (these being the bottom left and top right corners); the external angle of one of these sharp corners is indicated with a doubled headed curved arrow. At least one, or all, of the external corners of the primary patch portion and/or removable patch portion, or all removable patch portions, can have external angles of less than 315 degrees or 225 degrees. Alternatively, or in addition, at least one, or all, external corners of the primary patch portion and/or removable patch portion, or all removable patch portions may comprise a radius of at least 0.3 mm or 3 mm. The circle defining the corner radius is shown in dashed lines; despite being shown in dashed lines, it is not a removable patch portion. External corners of the adhesive patch having an external angle of greater than 315 degrees may be provided with a radius of greater than 0.5 mm.

### Anti-Stick Layer

Any aspect of the present disclosure may comprise an anti-stick layer configured to face upwards in use (e.g. away from the skin or body) or in some scenarios downwards and towards the skin. An anti-stick layer is particularly useful for preventing an overpatch from sticking to an already secured on-body device or on-body device patch when it is applied. As the overpatch may be intended for more frequent changing than the on-body device patch, it is advantageous to ensure that the overpatch does not adhere, or only minimally adheres, to the on-body device or on-body device patch as removal of the overpatch may otherwise remove the on-body device or weaken its adherence to the skin.

The anti-stick layer is usefully applied to the on-body device patch and may be applied to the upper surface of the on-body device or to the upper surface of the patch portions described above (e.g. the primary patch portion and/or any or all of the removable patch portions). The anti-stick layer may take the form of a permanent coating, may be a result of the material used to make the on-body device or the primary patch portion and/or any or all of the removable patch portions, or may take the form of a sticker that is applied to the on-body device or the primary patch portion and/or any or all of the removable patch portions. If the anti-stick layer is a sticker, the adhesive used for the sticker may be relatively weak as it needs only temporarily stay in place while an overpatch is placed on top of it; once the overpatch has been placed over it, it will be held in place by the overpatch. As such, the adhesive used for the sticker may be weaker than the adhesive used to form the adhesive layers of any one, or all, the patch portions.

By applying, or otherwise including, an anti-stick layer on the upper surface of any of the on-body device, the primary patch portion and/or any or all of the removable patch portions, the overpatch may be applied more easily over the existing on-body device patch. Moreover, the overpatch may be provided as a substantially continuous patch (that is, without an aperture for passing over the existing on-body device). The overpatch can simply be placed over the top of an existing on-body device patch and secured to the skin surrounding the on-body device patch; since at least some parts of the on-body device patch have an upwards facing anti-stick component, they will not adhere to the overpatch regardless of where the overpatch is positioned.

In general, a method of applying an overpatch may comprise an optional first step of applying an adhesive patch assembly comprising an on-body device (e.g. an on-body device patch) to a user's body, optionally using an applicator tool, and a second step of applying an overpatch over the top of the on-body device patch, optionally wherein an aperture or mating feature of the overpatch is positioned with respect to the on-body device. Where an anti-stick layer is provided, the optional step of positioning the aperture or mating feature of the overpatch may be dispensed with (although this may be dispensed with even without use of an anti-stick layer). Where the anti-stick layer is provided as a sticker, the method may comprise a further step between the first and second steps, wherein the anti-stick layer sticker is adhered to at least one of the upper surface of the on-body device, of the primary patch portion and/or of any or all of the removable patch portions.

The anti-stick layer may comprise or may be formed of a fluoropolymer or silicone. The anti-stick layer may comprise or may be formed of Teflon or a non-Teflon fluoropolymer.

As stated, each of the adhesive patch assemblies described can have various different shapes, sizes, manners of fabrication, and manners of placement. Each may be configured as an on-body device patch or as an overpatch.

### Reinforcement Devices

Referring back to the minimisation of adhesive patch peel away, in other approaches, a reinforcing element (e.g. a leg, ring or band) may extend from, e.g., the on-body device to contact the upper surface of the edge of the patch. The reinforcing element may simply rest on the upper surface or it may apply a force to the upper surface. In either case, the reinforcing element acts to hold down the edge and prevent it from peeling. In some aspects, this may result in the centre of the patch being raised slightly (as a reaction force); however, the centre of the patch is less likely to peel as it is not exposed, thus this slight reaction force is not problematic.

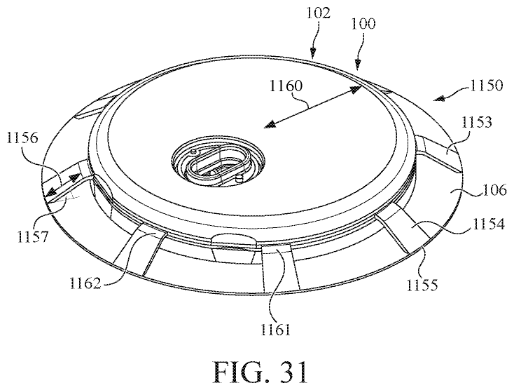

FIGS. 31-40B show reinforcement devices 1250 that are configured to reinforce the attachment of an adhesive patch 106 to a skin surface. The adhesive patch 106 is coupled to a base surface 104 of a housing 102 of an OBD 100. The reinforcement device 1250 comprises one or more reinforcing members. Reinforcing the adhesive patch 106 in this way may increase the period of time for which an OBD 100 can be used. These reinforcement devices may be used with any of the preceding OBDs and/or adhesive patches.

### Reinforcement Legs

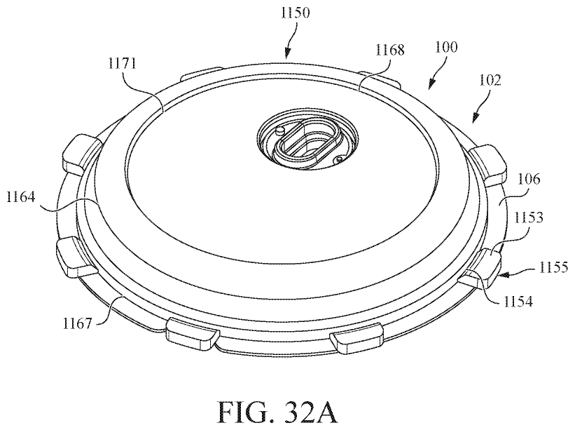

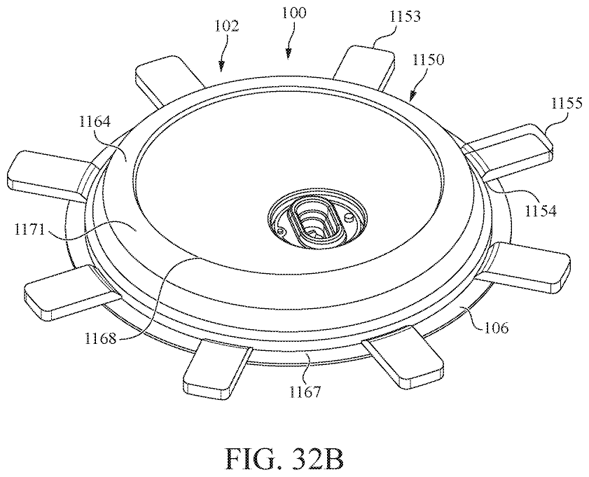

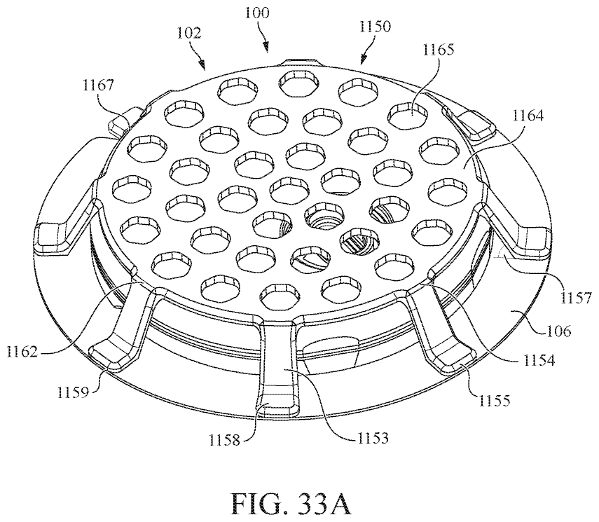

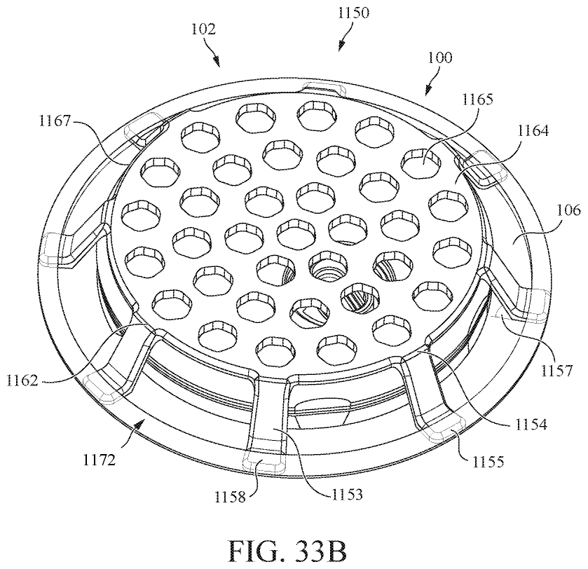

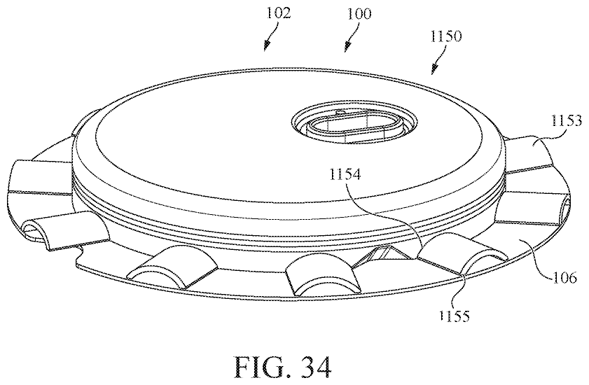

Referring to FIGS. 31-34, the reinforcement members comprise a plurality of reinforcement legs 1153. Each reinforcement leg 1153 comprises a first end 1154 and a second end 1155. The first end 1154 is configured to be directly or indirectly coupled to the housing 102. In other words, the first end 1154 of each leg 1153 may be configured to be directly coupled to the housing 102 as illustrated in FIG. 31 and FIG. 34. Alternatively, and as illustrated in FIGS. 32A-33B, the first end 1154 of each leg 1153 may be configured to directly couple to a body portion 1164 wherein the body portion 1164 is configured to be directly coupled to the housing 102 such that the first end 1154 of each leg 1153 is indirectly coupled to the housing. In the embodiments illustrated in FIGS. 31-34, the second end 1155 of each leg is configured to contact the adhesive patch 106 to thereby reinforce the attachment between the adhesive patch 106 and the skin surface.

The reinforcement device 1250 may comprise at least 4, at least 8, at least 10, at least 12, at least 16, or at least 20 reinforcement legs 1153. The reinforcement device 1250 may comprise an odd number of reinforcement legs 1153 or an even number of reinforcement legs 1153. The housing 102 or body portion 1164 comprises the same number of coupling sites 1161 as reinforcement legs 1153 that are configured to be coupled to the housing 102 or body portion 1164. Each coupling site 1161 is configured to allow a second end 1155 of a reinforcement leg 1153 to be coupled to the coupling site 1161.

In the embodiments illustrated in FIGS. 31-34, the reinforcement legs 1153 are configured to be disposed around the perimeter of the OBD 100. The reinforcement legs 1153 are configured to be spaced evenly around the perimeter of the housing 102. The coupling sites 1161 of the body portion 1164 or housing 102 are spaced evenly around the perimeter of the body portion 1164 or the housing 102. Even spacing of the reinforcement legs 1153 ensures that reinforcement of the adhesive patch 106 to the skin surface is delivered evenly around the adhesive patch. Alternatively, the reinforcement legs 1153 may be unevenly spaced to give a desired reinforcement profile.

Each reinforcement leg 1153 defines a respective longitudinal axis 1156 extending from the first end 1154 to the second end 1155 of each reinforcement leg 1153. Each axis 1156 may be oriented parallel to the base surface 104 of the housing 102 as illustrated in FIGS. 32A, 32B, and 34. Alternatively, each axis 1156 may be oriented at an angle 1157 to the base surface 104 as illustrated in FIGS. 31, 33A, and 33B. The angle 1157 may be between 0 and 90 degrees. The angle 1157 may be between 15 and 75 degrees. The angle 1157 may be between 30 and 60 degrees. The angle 1157 may be between 40 and 50 degrees. The angle 1157 may be 90 degrees in which case each axis may be parallel to an axis of the side portion of the OBD. A projection of each axis 1156 onto a plane parallel to the basis surface 104 of the housing 102 may be parallel to a respective radial axis 1160 that extends radially from a center of the base surface 104 of the housing and parallel to the base surface 104. Alternatively, a projection of each axis 1156 onto a plane parallel to the basis surface 104 of the housing 102 may be tangential to the housing 102.

At least one of the plurality of reinforcement legs 1153 may be configured to apply a downwards force on the adhesive patch 106 to thereby push the adhesive patch 106 towards the skin surface and reinforce the adhesive. The downward force may be applied in the event of peel-away of the adhesive patch 106, to prevent further peeling. In some embodiments the reinforcement legs 1153 are biased to exert a downward force on the adhesive patch 106. The reinforcement legs 1153 may be biased by a spring member. Alternatively or in addition, the reinforcement legs 1153 have a length such that they are configured to bend when coupled to the housing 102 or body portion 1164 and applied to the skin. This has the effect that, when the reinforcement legs are coupled to the housing 102 or body portion 1164, they are pre-tensioned and exert a downwards force on the adhesive patch 106.

The legs may be formed of a material comprising a rigid plastic or polymer such as a polycarbonate. In another embodiment, the legs may be formed from a material comprising an elastomer. In another embodiment, the legs may be formed from a material comprising metal. In another embodiment, the legs may be formed from a composite material comprising a polymer and a metal.

### Reinforcement Device of FIG. 31

In the embodiment illustrated in FIG. 31, each reinforcement leg 1153 is configured to be coupled directly to the housing 102 of the on-body device at a first end 1154 of the reinforcement leg 1153. Each reinforcement leg 1153 may correspond to any of the reinforcement legs 1153 described herein in the section titled “REINFORCEMENT LEGS”. Similarly, the features described herein in relation to the reinforcement device 1250 of FIGS. 32A-34 may be applied to the reinforcement device 1250 of FIG. 31.

In the embodiment illustrated in FIG. 31, the axis of each reinforcement leg 1156 is oriented at an angle 1157 to the base surface 104. The angle 1157 may be between 0 and 90 degrees. The angle 1157 may be between 15 and 75 degrees. The angle 1157 may be between 30 and 60 degrees. The angle 1157 may be between 40 and 50 degrees. A projection of each axis 1156 onto a plane parallel to the basis surface 104 of the housing 102 is parallel to a respective radial axis 1160 that extends radially from a center of the base surface 104 of the housing and in the plane of the base surface 104.

In the embodiment illustrated in FIG. 31, the reinforcement legs 1153 are supplied pre-attached to the housing 102. In particular, the reinforcement legs 1153 are formed integrally with the housing 102. The reinforcement legs 1153 may be coupled to the housing 102 by a living hinge 1162 or they may be coupled to the housing at a fixed angle. Using a reinforcement leg 1153 that is coupled to the housing 102 at a fixed angle may increase the force that the reinforcement leg 1153 is able to apply to the adhesive patch 106. In use, the user would first apply the OBD 100 comprising the adhesive patch 106 to the skin surface. In some implementations, the user may then position each reinforcement leg 1153 to ensure that the second end 1155 comes into contact with the adhesive patch 106. Pre-attachment of the reinforcement legs 1153 to the housing 102 in this way means that deployment of the reinforcement legs is simple and easy for the user. The skilled person would understand that there are many different types of connections that would facilitate the coupling between the housing and the reinforcement legs 1153. Coupling of the reinforcement legs 1153 directly to the housing 102 reduces the number of components required compared to embodiments where the reinforcement legs 1153 are coupled to a body portion 1164.

In other embodiments the reinforcement legs 1153 are not supplied pre-attached to the housing 102. The reinforcement legs 231 may be configured to be coupled to the housing by connectors. The connectors may be snap-fit connectors. The connectors may be hinges 1162. In use, the user would first apply the OBD 100 comprising the adhesive patch 106 to the skin surface using an applicator. The user would then attach each reinforcement leg 1153 to the housing 102 at a respective coupling site 1161 and manipulate the second end 1155 of each reinforcement leg 1153 to contact the adhesive patch 106. In embodiments wherein the couplings comprise a hinge 1162, manipulation of each reinforcement leg 1153 may comprise unfolding each hinge 1162 until the second end 1155 of each reinforcement leg 1153 comes into contact with the adhesive patch 106. Use of reinforcement legs 1153 which are not pre-attached to the housing 102 makes the reinforcement device 1250 more adaptable because different reinforcement legs 1153 may be selected to correspond with a user's requirements. For instance, the user may prefer to use smaller, more compact reinforcements legs 1153 for a more subtle visual appearance. The user may instead prioritize using reinforcement legs that apply a maximum force to the adhesive patch 102 in spite of a bulkier appearance. Furthermore, the user may opt to not apply a reinforcement leg 1153 at each coupling site 1161. Overall, using reinforcement legs 1153 that are not pre-attached provides a more modular reinforcement device which is more adaptable to user requirements and preferences.

### Reinforcement Devices of FIGS. 32A and 32B

In the embodiments illustrated in FIG. 32A and FIG. 32B, each reinforcement leg 1153 is configured to be coupled directly to a body portion 1164 at a first end 1154 of the reinforcement leg 1153. Each reinforcement leg 1153 may correspond to any of the reinforcement legs 1153 described herein in the section titled “REINFORCEMENT LEGS”. Similarly, the features described in relation to the reinforcement device 1250 of FIGS. 31, 33A-34 may be applied to the reinforcement device 1250 of FIG. 32A or the reinforcement device 1250 of FIG. 33B.

The skilled person would understand that there are many different types of connections that would facilitate the coupling of each reinforcement leg 1153 to the body portion 1164. Coupling of the reinforcement legs 1153 to the housing 102 via a body portion 1164 provides a more modular reinforcement device which is more adaptable to user requirements and preferences as the user may select a body portion 1164 that is best adapted for their requirements and preferences.

In the embodiments illustrated in FIGS. 32A and 32B, the reinforcement legs 1153 are pre-attached to the body portion 1164. In particular, the reinforcement legs 1153 may be formed integrally with the body portion 1164. Each reinforcement leg 1153 may be coupled to the body portion 1164 by a hinge 1162. The hinge 1162 may be a living hinge. In use, the user would first apply the OBD 100 comprising the adhesive patch 106 to the skin surface using an applicator. The user would then couple the body portion 1164 to the housing 102. The user may then unfold each reinforcement leg 1153 using the hinges 1162 until the second end 1155 comes into contact with the adhesive patch 106. Pre-attachment of the reinforcement legs 1153 to the body portion 1164 in this way means that deployment of the reinforcement legs is simple and easy for the user.

In other embodiments the reinforcement legs 1153 are not supplied pre-attached to the housing 102. The reinforcement legs 231 may be configured to be coupled to the body portion 1164 by connectors. The connectors may be snap-fit connectors. The connectors may be hinges 1162. In use, the user would first apply the OBD 100 comprising the adhesive patch 106 to the skin surface using an applicator. The user would then couple the body portion 1164 to the housing 102. The user would then attach each reinforcement leg 1153 to the body portion 1164 at a respective coupling site 1161 and manipulate the second end 1155 of each reinforcement leg 1153 to contact the adhesive patch 106. Alternatively, the user may first apply the OBD 100, then attach each reinforcement leg 1153 to the body portion 1164, then couple the body portion 1164 to the housing 102, and then may manipulate the second end 1155 of each reinforcement leg 1153 to contact the adhesive patch 106 if they do not already do so. In embodiments wherein the couplings comprise a hinge 1162, manipulation of each reinforcement leg 1153 may comprise unfolding each hinge 1162 until the second end 1155 of each reinforcement leg 1153 comes into contact with the adhesive patch 106. Use of reinforcement legs 1153 which are not pre-attached to the body portion 1164 makes the reinforcement device 1250 more adaptable because different reinforcement legs 1153 may be selected to correspond with a user's requirements. For instance, the user may prefer to use smaller, more compact reinforcements legs 1153 for a more subtle visual appearance. The user may instead prioritize using reinforcement legs that apply a maximum force to the adhesive patch 102 in spite of a bulkier appearance. Furthermore, the user may opt to not apply a reinforcement leg 1153 at each coupling site 1161. Overall, using reinforcement legs 1153 that are not pre-attached provides a more modular reinforcement device which is more adaptable to user requirements and preference.

In the embodiments of FIGS. 32A and 32B the axis 1156 of each reinforcement leg 1153 is oriented parallel to the base surface 104 of the housing 102. Using reinforcement legs 1153 that extend parallel to the base surface of the housing means that a greater proportion of each leg is in contact with the adhesive patch 106 and therefore the reinforcement of the adhesive patch 106 is more evenly distributed over the surface of the adhesive patch 106.

In the embodiment of FIG. 32A the second end 1155 of each reinforcement leg 1153 terminates at the periphery of the adhesive patch 106. In the embodiment of FIG. 32B the second end 1155 of each reinforcement leg 1153 extends radially beyond the periphery of the adhesive patch. Using longer reinforcement legs may be more effective at preventing peeling. However, longer reinforcement legs may increase the visual appearance of the OBD 100, which may be undesirable to the user.

In the embodiments illustrated in FIG. 32A and FIG. 32B the body portion 1164 defines an opening 1167 configured to partially or fully receive the housing 102. The opening 1167 is bounded by an inner edge 1168 and the body portion also defines an outer edge 1167.

The inner edge 1168 has a shape corresponding to the perimeter of the housing 102. In the illustrated example the shape of the inner edge is a circular shape. However, in other embodiments the shape of the inner edge 1168 may be a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The shape of the inner edge 1168 may comprise sharp and/or rounded corners.

The shape of the inner edge 1168 may comprise straight and/or curved edges. At least some of the curved edges may have a constant radius of curvature. At least some of the curved edges may have a variable radius of curvature. The shape of the inner edge 1168 may comprise one or more indentations and/or one or more protrusions or lobes.

In the embodiment of FIGS. 32A and 32B the outer edge 1167 has the same shape as the inner edge 1168. However, in other embodiments the shape of the outer edge 1167 may be different to the shape of the inner edge 1168. The shape of the outer edge 1167 may be a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The shape of the outer edge 1167 may comprise sharp and/or rounded corners. The shape of the outer edge 1167 may comprise straight and/or curved edges. At least some of the curved edges may have a constant radius of curvature. At least some of the curved edges may have a variable radius of curvature. The shape of the outer edge 1167 may comprise one or more indentations and/or one or more protrusions or lobes.

In the embodiment of FIGS. 32A and 32B the body portion 1164 is configured to be coupled to the housing 102. The skilled person would understand that there are many different types of coupling that could be used to achieve this. The body portion 1164 may be configured to be coupled to the housing 102 by a snap fit joint 1166 that extends around a perimeter of the body portion 1164 and a perimeter of the housing 102. Alternatively, the body portion 1164 can be configured to be coupled to the housing 102 by a plurality of snap fit joints 1166 disposed around a perimeter of the body portion 1164 and a perimeter of the housing 102. In another embodiment the body portion 1164 may be formed from an elastomeric material. The inner opening 1167 may be sized such that the elastomeric body portion 1164 may grip the housing 102 to thereby couple the body portion 1164 to the housing 102. Alternatively, the body portion 1164 may comprise a lip extending around the inner opening 1167 and formed of an elastomeric material and sized such that the lip 1171 may grip the housing 102 to thereby couple the body portion 1164 to the housing 102. In another embodiment the body portion 1164 may be formed from a shape memory material. The inner opening 1167 may be sized such that the shape memory body portion 1164 may grip the housing 102 to thereby couple the body portion 1164 to the housing 102. Alternatively, the body portion 1164 may comprise a lip 1171 extending around the inner opening 1167 formed of an elastomeric material and sized such that the lip 1164 may grip the housing 102 to thereby couple the body portion 1164 to the housing 102. In another embodiment the body portion 1164 may be coupled to the housing 102 by an adhesive applied to the upper surface and/or the side surface of the housing 102.

The body portion 1164 may comprise an anti-stick layer as described herein in the section titled “ANTI-STICK LAYER”. For instance, the anti-stick layer may be provided on the upper surface of the body portion 1164.

### Reinforcement Devices of FIGS. 33A and 33B

The embodiments illustrated in FIGS. 33A and 33B combine some of the features of the embodiment of FIG. 31 with some of the features of FIG. 32A. In particular, the embodiments of FIGS. 33A and 33B comprise a body portion to which the reinforcement legs are coupled, similarly to that of FIG. 32A, with reinforcement legs oriented at an angle to the adhesive patch, similarly to the reinforcement legs of FIG. 31.

In the embodiments illustrated in FIGS. 33A and 33B the body portion differs from that of FIG. 32A in that the body portion 1164 does not comprise an inner opening 1167. Instead, the body portion 1164 is configured to cover an upper surface of the housing 102. The body portion 1164 is configured to cover the entire upper surface of the housing 102. The body portion 1164 takes the form of a plate.

The plate comprises an outer edge 1167. In the embodiment of FIGS. 33A and 33B the outer edge 1167 has the same shape as the housing which in the illustrated embodiment is circular. In other embodiments, the shape of the outer edge 1167 may be a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The shape of the outer edge 1167 may comprise sharp and/or rounded corners. The shape of the outer edge 1167 may comprise straight and/or curved edges. At least some of the curved edges may have a constant radius of curvature. At least some of the curved edges may have a variable radius of curvature. The shape of the outer edge 1167 may comprise one or more indentations and/or one or more protrusions or lobes.

The body portion 1164 is configured to be coupled to the housing 102. The skilled person would understand that there are many different types of coupling that could be used to achieve this. For example, the body portion 1164 may be configured to be coupled to the housing 102 by a snap fit joint 1166 that extends around a perimeter of the body portion 1164 and a perimeter of the housing 102. Alternatively, the body portion 1164 can be configured to be coupled to the housing 102 by a plurality of snap fit joints 1166 disposed around a perimeter of the body portion 1164 and a perimeter of the housing 102. In another embodiment at least a portion of the body portion 1164 may be formed from an elastomeric material. The plate may comprise a lip 1171 formed from an elastomeric material extending from the outer edge 1167. The lip 1167 may be sized such that it may grip the housing 102 to thereby couple the body portion to the housing. In another embodiment at least a portion of the body portion 1164 may be formed from a shape memory material. The lip 1171 may be formed from a shape memory material and be sized such that the lip 1171 may grip the housing 102 to thereby couple the body portion 1164 to the housing 102. In another embodiment the body portion 1164 may be coupled to the housing 102 by an adhesive applied to the upper surface and/or the side surface of the housing 102.

The body portion 1164 may be provided with a plurality of apertures 1165. The apertures may extend entirely through the body portion 1164. The apertures 1165 may be configured to allow moisture to escape from under the body portion 1164. This has the advantage of increasing the length of time for which the adhesive patch 106 remains adhered to the skin surface because the presence of moisture can cause the adhesive to fail and the patch 106 to peel away from the skin surface. The body portion 1164 may be formed from a moisture permeable material which may have a similar effect. The body portion 1164 may be formed from the same material as the reinforcement legs 1153. The body portion 1164 may be formed form a material comprising a rigid plastic or polymer such as a polycarbonate. In another embodiment, the body portion 1164 may be formed from a material comprising an elastomer. In another embodiment, the body portion 1164 may be formed from a material comprising metal. In another embodiment, the body portion 1164 may be formed from a composite material comprising a polymer and a metal.

When applied to the skin surface, the body portion 1164 may at least partially cover one or more of the reinforcement legs 1153. The body portion 1164 may be configured to fully cover the legs 1153.

In the embodiments illustrated in FIGS. 33A and 33B the second end 1155 of each reinforcement leg 1153 comprise a foot 1158. At least a portion of each foot 1158 is configured to contact the adhesive patch 106. Each foot 1158 may be configured to extend beyond a periphery of the adhesive patch such that at least a portion of each foot 1158 may be configured to contact the skin surface. Each foot 1158 may be oriented at an angle to its respective refinement leg 1153 as illustrated in FIGS. 33A and 33B or the foot 1158 may be parallel to its respective reinforcement leg 1153. The angle between each foot 1158 and each reinforcement leg 1153 may be between 90 and 180 degrees. The angle between each foot 1158 and each reinforcement leg 1153 may be between 115 and 165 degrees. The angle between each foot 1158 and each reinforcement leg 1153 may be between 120 and 150 degrees. The angle between each foot 1158 and each reinforcement leg 1153 may be between 130 and 140 degrees. The angle between each foot 1158 and each reinforcement leg 1153 may be 90 degrees.

Each foot 1158 may comprise an adhesive layer 1159. The adhesive layer 1159 of each foot 1158 may be configured to adhere to an upper surface of the adhesive patch 106. This embodiment ensures that contact between the foot 1158 and the adhesive patch 106 is maintained even if the OBD 100 is knocked or the skin surface under the base surface 104 of the housing 102 moves.

When applied to the skin surface, the body portion 1164 may at least partially cover one or more of the reinforcement legs 1153. The body portion 1164 may be configured to fully cover the legs 1153.

In the embodiments illustrated in FIG. 33B, the feet 1158 of each reinforcement leg 1153 are coupled to each other by a connection member 1172. The connection member 1172 is configured to be positioned around the perimeter of the adhesive patch 106. The connection member 1172 is configured to overlay the adhesive patch 106. The shape of the outer edge of the connection member 1172 corresponds to the shape of the adhesive patch 106 such that the outer edge of the connection member is configured to overlay the periphery of the adhesive patch 106.

The connection member 1172 may comprise an adhesive layer disposed on a lower surface and configured to adhere to an upper surface of the adhesive patch 106. The adhesive layer may be further configured to adhere to the feet 1158 of the reinforcement legs 1153. Alternatively, the connection member 1172 may be configured to be adhered to the adhesive patch 106 by adhesive disposed on an upper surface of the adhesive patch 106. In use, the user would apply the connection member 1172 to the adhesive patch 106 after manipulating the reinforcement legs 1153 to contact the adhesive patch 106. In another embodiment the connection member 1172 may be formed integrally with the feet 1158 of the reinforcement legs 1153 or directly with the reinforcement legs 1153 themselves.

The connection member 1172 illustrated in FIG. 33B defines an inner edge. The inner edge has a shape corresponding to the perimeter of the housing 102 and/or the shape of the adhesive patch 106. In the embodiment illustrated in FIG. 33B the shape of the inner edge is circular. However, in other embodiments the shape of the inner edge may be a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The shape of the inner edge may comprise sharp and/or rounded corners. The shape of the inner edge may comprise straight and/or curved edges. At least some of the curved edges may have a constant radius of curvature. At least some of the curved edges may have a variable radius of curvature. The shape of the inner edge may comprise one or more indentations and/or one or more protrusions or lobes.

In the embodiment illustrated in FIG. 33B the outer edge has the same shape as the inner edge. In other embodiments the outer edge and inner edge may have different shapes. In either case, the shape of the outer edge may be a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The shape of the outer edge may comprise sharp and/or rounded corners. The shape of the outer edge may comprise straight and/or curved edges. At least some of the curved edges may have a constant radius of curvature. At least some of the curved edges may have a variable radius of curvature. The shape of the outer edge may comprise one or more indentations and/or one or more protrusions or lobes.

The connection member 1172 may be discontinuous. That is, there may be one or more gaps extending between the inner and outer edges. The connection member 1172 may therefore be “C” shaped. Alternatively, the connection member 1172 may comprise a plurality of sub-connection members each configured to couple a subset of the feet 1158 together. In one example, each sub-connection member couples two neighbouring feet together.

The connection member 1172 may be comprised by the body portion 1164. That is, the connection member 1172 may be directly coupled to or formed integrally with the body portion 1164.

The connection member may be integrally formed with the reinforcement legs 1153. In use, the user may first apply the OBD 100 comprising the adhesive patch 106 to the skin surface using an applicator. The user may then couple a body portion 1164 to the housing 102. The user would then apply the connection member 1172 to the adhesive patch 106 before manipulating the reinforcement legs 1153 to couple them to the housing 102 or body portion 1164.

### Reinforcement Device of FIG. 34

In the embodiment illustrated in FIG. 34, each reinforcement leg 1153 has an arcuate cross section. Each reinforcement leg 1153 may correspond to any of the reinforcement legs 1153 described herein in the section titled “REINFORCEMENT LEGS” with the addition of the arcuate cross section. Similarly, the features described in relation to the reinforcement device 1250 of FIGS. 31-33B may be applied to the reinforcement device 1250 of FIG. 34. The reinforcement legs 1153 are arranged such that they are convex relative to the adhesive patch 106. The material from which the reinforcement legs 1153 are formed is relatively stiff. This means that each reinforcement leg 1153 is configured to bend along its longitudinal axis 1156 towards the skin surface but resists against bending in the opposite direction away from the skin surface. This may be achieved with a bistable configuration, in a similar manner to a metallic tape measure. In this way, the reinforcement legs 1153 allow the adhesive patch 106 to conform to the skin surface of a user, which is generally convex, while resisting against peeling of the adhesive patch 106 away from the skin surface. This has the effect of reinforcing the attachment of the adhesive patch 106 to the skin surface.

Each reinforcement leg 1153 may be formed form a material comprising a rigid plastic or polymer such as a polycarbonate. In another embodiment, each reinforcement leg 1153 may be formed from a material comprising an elastomer. Each reinforcement leg 1153 may be formed form an elastomeric overmold. In another embodiment, each reinforcement leg 1153 may be formed from a material comprising metal. In another embodiment, the arcuate channels may be formed from a composite material comprising a polymer and a metal.

### Reinforcement Bands

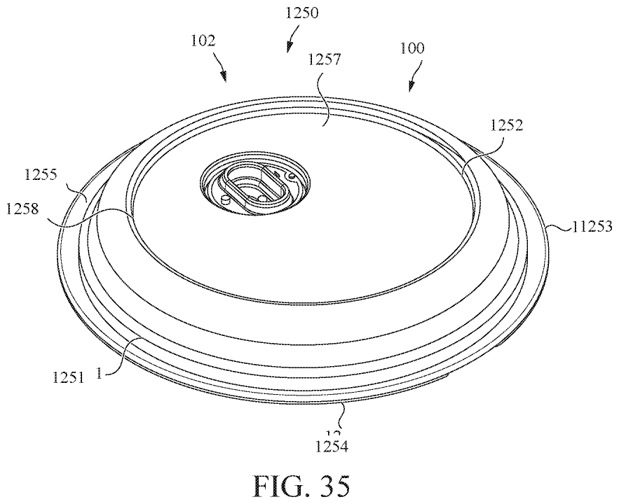

In the embodiments illustrated in FIGS. 35-40B the reinforcement device 1250 comprises one or more reinforcement bands 1251. Each of the one or more reinforcement bands comprises an inner edge 1251 and an outer edge 1252. Between the inner edge 1251 and outer edge 1252 is a lower surface 1254 configured to overlay the adhesive patch 106. The inner edge 1251 defines an opening 1257.

The inner edge 1251 may have a shape corresponding to the perimeter of the housing 102 and/or the shape of the adhesive patch 106. The shape of the inner edge 1251 may be a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The shape of the inner edge 1251 may comprise sharp and/or rounded corners. The shape of the inner edge 1251 may comprise straight and/or curved edges. At least some of the curved edges may have a constant radius of curvature. At least some of the curved edges may have a variable radius of curvature. The shape of the inner edge 1251 may comprise one or more indentations and/or one or more protrusions or lobes.

The outer edge 1252 may have the same shape as the inner edge 1251. In other embodiments the outer edge 1252 and inner edge 1251 may have different shapes. In either case, the shape of the outer edge 1252 may be a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape. The shape of the outer edge 1252 may comprise sharp and/or rounded corners. The shape of the outer edge 1252 may comprise straight and/or curved edges. At least some of the curved edges may have a constant radius of curvature. At least some of the curved edges may have a variable radius of curvature. The shape of the outer edge 1252 may comprise one or more indentations and/or one or more protrusions or lobes.

The material of the reinforcement bands 1251 may be selected to provide varying rigidity, adhesion and/or moisture wicking properties. Each reinforcement band 1251 may be formed form a material comprising a rigid plastic or polymer such as a polycarbonate. In another embodiment, each reinforcement band 1251 may be formed from a material comprising an elastomer. In another embodiment, each reinforcement band 1251 may be formed from a material comprising metal. In another embodiment, each reinforcement band 1251 may be formed from a composite material comprising a polymer and a metal. The body portion 1164 may comprise an anti-stick layer as described in the section titled “ANTI-STICK LAYER”.

The reinforcement bands 1251 are configured to reinforce the adhesion between the adhesive patch 106 and the skin surface in order to prevent the peripheral edge of the adhesive patch 106 from peeling away. In some embodiments the one or more bands achieve by sitting against or overlapping the peripheral edge to prevent peeling. In other embodiments the one or more bands comprise features that resist against peeling itself.

The reinforcement band 1521 may be discontinuous. That is, there may be one or more gaps extending between the inner and outer edges. The reinforcement band 1251 may therefore be “C” shaped. Alternatively, the reinforcement band 1251 may comprise a plurality of sub-reinforcement band 1251 each configured to extend a portion of the way around the perimeter of the housing. An example of such an arrangement is described herein in the section “DISCRETE BEADS”.

### Single Reinforcement Band

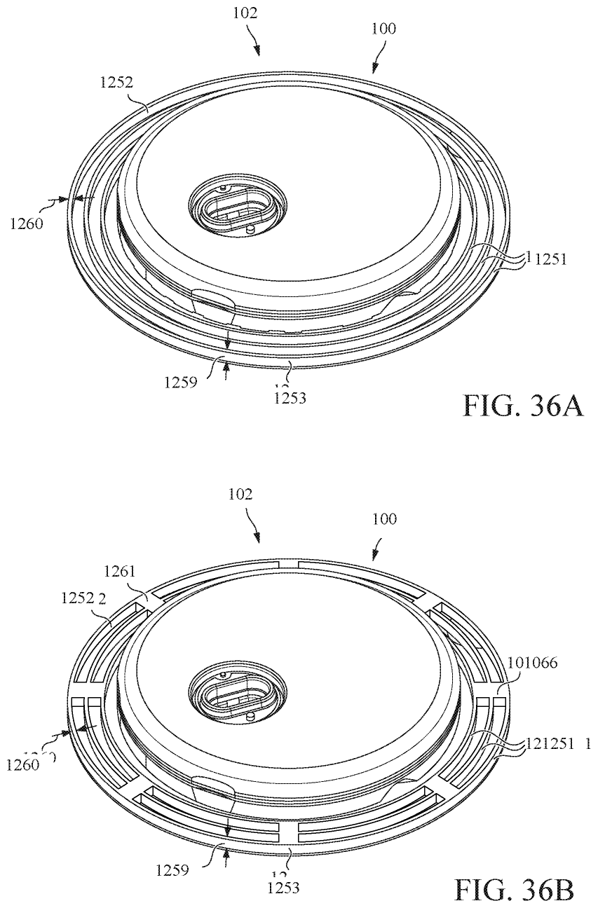

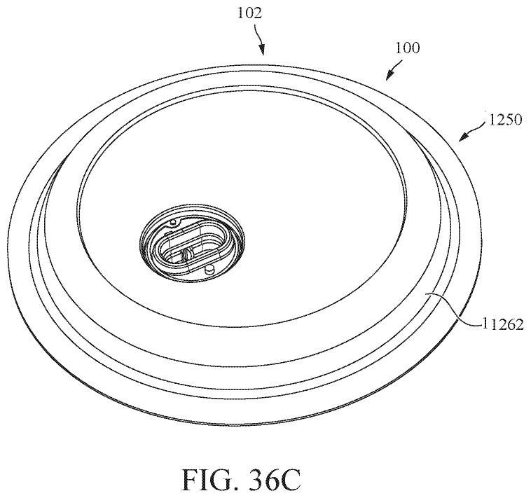

In the embodiment illustrated in FIG. 35 the reinforcement device 1250 comprises exactly one reinforcement band 1251. The reinforcement band 1251 may correspond to any of the reinforcement bands 1251 described herein in the section titled “REINFORCEMENT BANDS”. Similarly, the features described in relation to the reinforcement device 1250 of FIGS. 36A to 40B may be applied to the reinforcement device 1250 of FIG. 35.

The outer edge 1252 of the reinforcement band 1251 is configured to overlie a peripheral edge of the adhesive patch 106. In other words, the radius of the outer edge 1252 corresponds to the radius of the peripheral edge of the adhesive patch 106. In this way, the reinforcement band 1251 reinforces the adhesion of the adhesive patch 106 to the skin surface and prevents the commencement of the adhesive patch 106 peeling away from the skin surface. It is desirable to avoid the commencement of any peeling away, as a peeling edge can propagate readily, particularly as the exposed adhesive can catch on clothing or other materials, leading to additional shear forces being applied to the housing of the on-body device and adhesive patch 106. Alternatively, the outer edge 1253 of the reinforcement band 1251 may extend radially beyond the periphery of the adhesive patch 106 such that the outer edge 1253 directly contacts the skin surface.

In this embodiment the reinforcement band 1251 is sized such that the inner edge 1251 overlies an upper surface of the housing 102. However, the inner edge 1251 may be sized to have a larger radius than the housing 102 such that the housing 102 extends through the opening 1257. The inner edge 1251 comprises a lip 1258 around the opening 1257 and may be formed of an elastomeric material and sized such that the lip 1258 may grip the housing 102 to couple the reinforcement band 1251 to the housing 102. Alternatively, the lip 1258 may comprise a snap fit joint portion that extends around a perimeter of the inner edge 1251 and which is configured to couple to a corresponding joint portion that extends around a perimeter of the housing 102.

The lower surface 1254 may comprise an adhesive layer configured to adhere to an upper surface of the adhesive patch 106 and/or the skin of the user. Alternatively, the adhesive patch 106 may comprise an adhesive upper surface to which the reinforcement band can be adhered.

### Concentric Tall Bands

In the embodiments illustrated in FIGS. 36A to 36C, the one or more reinforcement bands comprises a plurality of reinforcement bands 1251 arranged concentrically. Each of the reinforcement bands 1251 may correspond to any of the reinforcement bands 1251 described herein in the section titled “REINFORCEMENT BANDS”. Similarly the features described in relation to the reinforcement device 1250 of FIG. 35, or 37 to 40B may be applied to the reinforcement device 1250 of FIGS. 36A to 36C.

In the embodiments of FIGS. 36A to 36C the outer edge 1252 of the outermost reinforcement band is configured to overlie a peripheral edge of the adhesive patch 106 so that the outermost reinforcement band 1251 reinforces the adhesion of the adhesive patch 106 to the skin surface. However, in other embodiments the outer edge 1253 of the outermost reinforcement band may extend radially beyond the periphery of the adhesive patch 106 such that the outer edge 1252 directly contacts the skin surface.

The reinforcement bands 1251 are formed from a flexible or semi-flexible material. Each reinforcement band is adhered to an upper surface of the adhesive patch 106. The lower surface 1254 of each reinforcement band may comprise an adhesive layer configured to adhere to an upper surface of the adhesive patch 106. Alternatively, the adhesive patch may comprise an adhesive upper layer configured to adhere the reinforcement bands 1251 to the upper surface of the adhesive patch 106. In use, the user may apply each reinforcement band to the upper surface of the adhesive patch 106 individually. Alternatively, the plurality of reinforcement bands 1251 may be arranged on a coupling layer. The coupling layer may comprise an adhesive surface on a surface opposite to the surface on which the reinforcement bands are arranged. Alternatively, the adhesive patch 106 may comprise an adhesive upper layer configured to adhere the coupling layer to the upper surface of the adhesive patch 106. In use, the user may apply the coupling layer to the adhesive patch to couple the reinforcement bands 1251 to the adhesive patch 106. Providing the reinforcement bands 1251 pre-attached to a coupling layer ensures that application of the reinforcement bands 1251 may be achieved more straightforwardly and with good concentric alignment of the reinforcement bands 1251.

Each reinforcement band 1251 defines a height 1259 and a width 1260. The height is defined between the lower surface of each band and an upper surface of each band in a direction perpendicular to the plane in which the band lies. The width is defined in a radial direction between the inner and outer edges. The ratio of the height 1259 to the width 1260 may be greater than 2. The ratio of the height 1259 to the width 1260 may be greater than 3. The ratio of the height 1259 to the width 1260 may be greater than 5. The ratio may be chosen such that concave peeling of the adhesive patch away from the skin surface causes the reinforcement bands 1251 to abut against one another thus preventing further peeling. The arrangement also allows concave flexing of the adhesive patch 106, thus allowing the adhesive patch to conform to the skin surface of the user even as the skin surface is moved.

In the embodiment illustrated in FIG. 36B, the reinforcement bands 1251 are connected to one another via webbing members 1261. The webbing members 1261 may extend radially between the concentric reinforcement bands 1251, thereby interlinking the reinforcement bands 1251 in a spiderweb pattern. The use of webbing members 1261 between the reinforcement bands 1251 ensures good concentric alignment of the reinforcement bands 1251 and mechanical stability thereof, thus making application of the reinforcement device 1250 easier.

In the embodiment illustrated in FIG. 36C the reinforcement bands 1251 are covered by a cover band 1262. The cover band 1262 may further protect the adhesive patch 106 from peeling by protecting the reinforcement bands 1251 from accidental knocks. The cover band 1262 may be similar to the reinforcement band 1251 described in the section titled “REINFORCEMENT BANDS”. The reinforcement bands 1251 may be coupled to the cover band 1262 either directly or via the webbing members 1261.

### Arcuate Channels

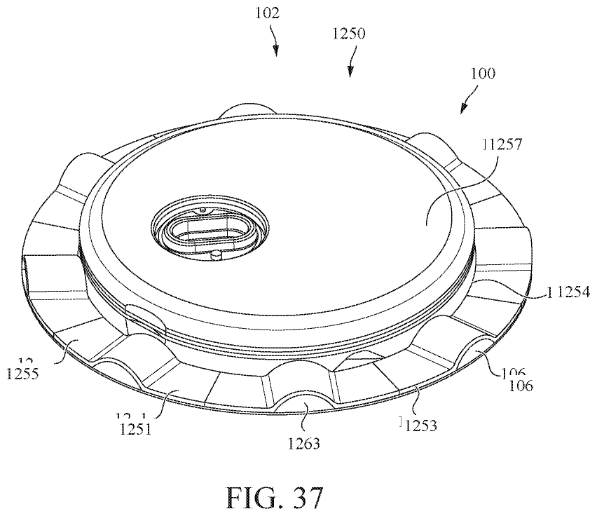

In the embodiment illustrated in FIG. 37, the reinforcement device 1250 comprises exactly one reinforcement band 1251. The reinforcement band 1251 may correspond to any of the reinforcement bands 1251 described herein in the section titled “REINFORCEMENT BANDS”. Similarly the features described in relation to the reinforcement device 1250 of FIGS. 35 to 36C or 38A to 40B may be applied to the reinforcement device 1250 of FIG. 37.

In particular, the reinforcement band 1251 of FIG. 37 is similar to the reinforcement band 1251 of FIG. 35 but additionally comprises a number of arcuate channels 1263 defined between the reinforcement band 1251 and the adhesive patch 106. The arcuate channels 1263 extend between the inner and outer edges of the reinforcement band 1251 in a radial direction. Each channel 1263 is convex with respect to the skin surface. Each channel 1263 may have a constant width. The material from which the channels 1263 are formed is relatively stiff. This means that each channel 1263 is configured to bend along its longitudinal axis towards the skin surface but resists against bending in the opposite direction (away from the skin surface). This may be achieved with a bistable configuration, in a similar manner to a metallic tape measure. In this way, the channels 1263 allow the adhesive patch 106 to conform to the skin surface of a user, which is generally convex, while resisting against peeling of the adhesive patch 106 away from the skin surface. This has the effect of reinforcing the attachment of the adhesive patch 106 to the skin surface.

The arcuate channels 1263 may be formed from the same material as the rest of the reinforcement band 1251 or a different material. The arcuate channels 1263 may be formed form a material comprising a rigid plastic or polymer such as a polycarbonate. In another embodiment, the arcuate channels 1263 may be formed from a material comprising an elastomer. The arcuate channels 1263 may be formed from an elastomeric overmold. In another embodiment, the arcuate channels 1263 may be formed from a material comprising metal. In another embodiment, the arcuate channels 1263 may be formed from a composite material comprising a polymer and a metal.

### Discrete Beads

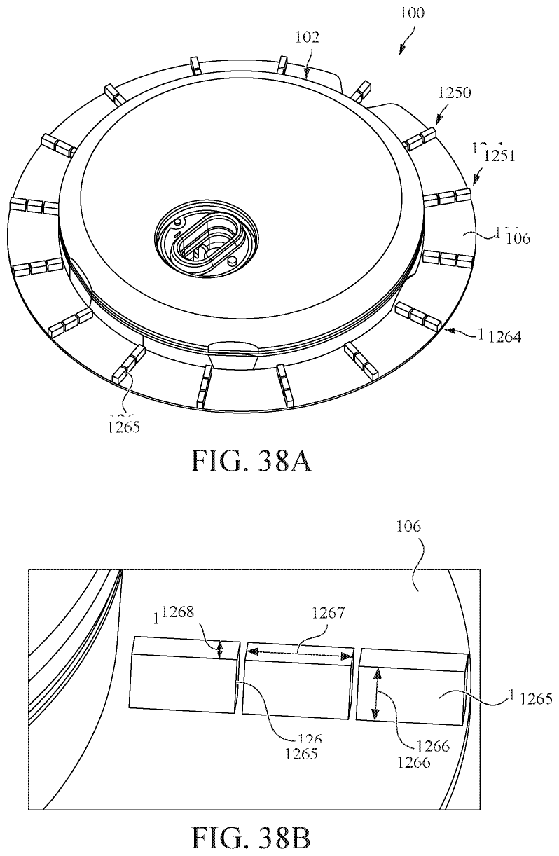

In the embodiment illustrated in FIGS. 38A and 38B each reinforcement band 1251 comprises a plurality of discrete beads 1264. Each reinforcement band 1251 comprises a number of discrete beads 1264 spaced evenly around the reinforcement band 1251. The plurality of reinforcement bands 1251 are arranged such that the beads 1264 of each reinforcement band 1251 are radially aligned. That is, the beads 1264 are arranged in radial rows. Each row of beads 1264 may comprise at least three beads. Each row of beads 1264 may comprise at least five beads. Each row of beads 1264 may comprise less than eight beads.

The beads are sized and positioned such that, upon concave flexing of the adhesive patch 106 adjacent beads abut against one another thus preventing further peeling of the adhesive patch 106. The faces 1265 of each bead that face towards a neighbouring bead 1264 are flat. In the embodiment illustrated in FIGS. 38A and 38B each bead 1264 is cuboidal with the longest axis aligned with the radial axis of the reinforcement band 1251. Alternatively, each bead 1264 may be prismatic with the longest axis aligned with the radial axis of the reinforcement band 1251.

The discrete beads 1264 may be covered by a cover band 1262 similar to the cover band described with reference to FIG. 36C. The cover band 1262 may further protect the adhesive patch 106 from peeling by protecting the reinforcement beads 1264 from accidental knocks or from being caught in the user's clothing. The cover band 1262 may be similar to the reinforcement band 1251 described in the section titled “REINFORCEMENT BANDS”.

The discrete beads 1264 may be individually adhered to the adhesive patch 106. Alternatively, long beads may be positioned on the adhesive patch with their longest axis radially aligned and adhered to the adhesive patch. The long beads can then be cut perpendicular to the radial axis to form the discrete beads 1264. In another embodiment, the discrete beads 1264 may be embossed onto the surface of the adhesive patch 106.

Each bead 1264 may be a cuboid and may define a height 1266, a length 1267, and a width 1268. Each bead 1264 may have a height 1266 defined in an axis perpendicular to the plane of the adhesive patch of less than 3 mm. Each bead 1264 may have a height 1266 of between 1 mm and 3 mm. Each bead 1264 may have a height 1266 of between 1.5 mm and 2.5 mm.

Each bead 1264 may have a width 1268 of between 1 mm and 3 mm. Each bead 1264 may have a width 1268 of between 1.5 mm and 2.5 mm.

Each bead 1264 may have a length 1267 of less than 22 mm. Each bead 1264 may have a length of between 1 mm and 22 mm. Each bead 1264 may have a length of between 1 mm and 10 mm. Each bead 1264 may have a length of between 2 mm and 5 mm.

In a preferred embodiment, each bead 1264 has a height 1266 of between 1 mm and 3 mm, a width 1268 of between 1 mm and 3 mm and a length 1267 of between 2 mm and 5 mm.

### Multiple Bands in Flat Concentric Arrangement

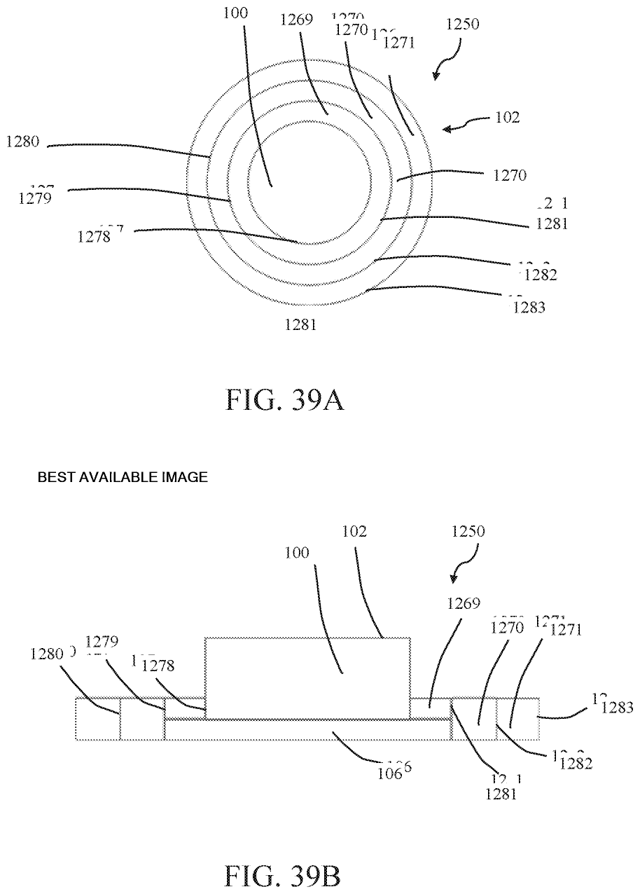

In the embodiment of FIGS. 39A and 39B the reinforcement device 1250 comprises a plurality of reinforcement bands 1251 arranged concentrically and with their lower surfaces co-planar. Each reinforcement band 1251 may correspond to any of the reinforcement bands 1251 described herein in the section titled “REINFORCEMENT BANDS”. Similarly, the features described in relation to the reinforcement device 1250 of FIGS. 35 to 38B or 40A to 40B may be applied to the reinforcement device 1250 of FIGS. 39A and 39B.

In the embodiment illustrated, there are 3 reinforcement bands 1251. However, the reinforcement device may comprise any number of reinforcement bands 1251. For example, the reinforcement device 1250 may comprise 2 reinforcement bands 1251, 4 reinforcement bands 1251, 5 reinforcement bands 1251, 6 reinforcement bands 1251, 7 reinforcement bands 1251, or 8 reinforcement bands 1251.

Each reinforcement band 1251 is frangibly connected to the neighbouring reinforcement band 1251. The innermost reinforcement band 1269 is the first reinforcement band 1269, the middle reinforcement band 1270 is the second reinforcement band 1270 and the outermost reinforcement band 1271 is the third reinforcement band 1271. The outer edge 1281 of the first reinforcement band 1269 is frangibly connected to the inner edge 1279 of the second reinforcement band 1270. The outer edge 1282 of the second reinforcement band 1270 is frangibly connected to the inner edge 1280 of the third reinforcement band 1271. In embodiments comprising a different number of reinforcement bands 1251, the outer edge 1252 of the nth reinforcement band 1251 is coupled to the inner edge 1251 of the n+1th reinforcement band 1251.

The outer edge 1281 of the first reinforcement band 1269 is configured to overlay the peripheral edge of the adhesive patch 106. In other words, the radius of the outer edge 1281 corresponds to the radius of the peripheral edge of the adhesive patch 106. Alternatively, the outer edge 1281 of the first reinforcement band 1269 may be configured to extend beyond the peripheral edge of the adhesive patch 106.

In use, if the outer edge 1283 of the outermost reinforcement band 1251 begins to peel away from the skin surface, the user can simply break the frangible connection between the third and second reinforcement bands and remove the third reinforcement band from the skin surface. This means that the peeling cannot spread to the second reinforcement band and thus ensures that the peripheral edge of the adhesive patch is well adhered to the skin surface. Similarly, if the outer edge 1282 of the second reinforcement band 1270 subsequently begins to peel away from the skin surface, the user can simply break the frangible connection between the second and first reinforcement bands and remove the second reinforcement band from the skin surface.

One or more of the plurality of reinforcement bands 1251 may be formed from a material comprising a rigid plastic or polymer such as a polycarbonate. In another embodiment, one or more of the plurality of reinforcement bands 1251 may be formed from a material comprising an elastomer. In another embodiment, one or more of the plurality of reinforcement bands 1251 may be formed from a material comprising metal. In another embodiment, one or more of the plurality of reinforcement bands 1251 may be formed from a composite material comprising a polymer and a metal.

The materials of the plurality of reinforcement bands 1251 may be selected to provide varying rigidity, adhesion and/or moisture wicking properties. In one embodiment the rigidity of the plurality of reinforcement bands 1251 decreases in a radial direction to ensure that the outermost bands 1251 can conform well with the skin surface. In one embodiment the adhesion of the plurality of reinforcement bands 1251 decreases in a radial direction to reduce the likelihood that an inner band is inadvertently removed when an outer band is removed from the skin surface. Furthermore, the inner bands are configured to remain attached to the skin surface for a longer duration and therefore may require stronger adhesion. Alternatively, the adhesion may increase in a radial direction to reduce the likelihood of the outer edge of the outermost band peeling away from the skin surface. The moisture wicking ability of the bands may decrease in a radial direction as moisture build up is more likely to be problematic under the inner bands which are designed to be applied to the skin for a longer time period.

### Multiple Bands Stacked

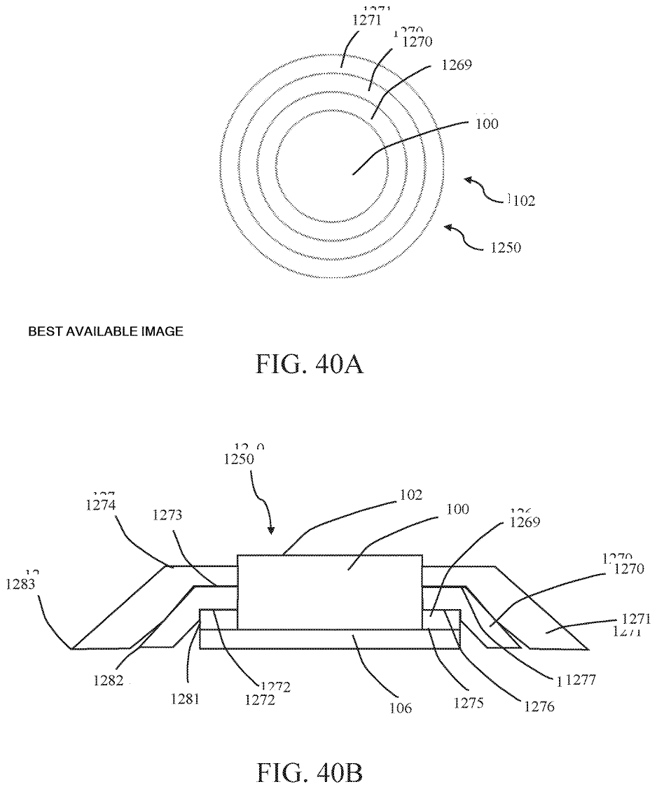

In the embodiment of FIGS. 40A and 40B the reinforcement device 1250 comprises a plurality of reinforcement bands 1251 arranged concentrically stacked on top of one another. Each reinforcement band 1251 may correspond to any of the reinforcement bands 1251 described herein in the section titled “REINFORCEMENT BANDS”. Similarly, the features described in relation to the reinforcement device 1250 of FIGS. 35 to 39B, may be applied to the reinforcement device 1250 of FIGS. 40A and 40B.

In the embodiment illustrated there are 3 reinforcement bands 1251. However, the reinforcement device may comprise any number of reinforcement bands 1251. For example, the reinforcement device 1250 may comprise 2 reinforcement bands 1251, 4 reinforcement bands 1251, 5 reinforcement bands 1251, 6 reinforcement bands 1251, 7 reinforcement bands 1251, or 8 reinforcement bands 1251.

Each reinforcement band 1251 is frangibly connected to the neighbouring reinforcement band 1251. The reinforcement band 1269 closest to the skin surface is the first reinforcement band 1269, the middle reinforcement band 1270 is the second reinforcement band 1270 and the reinforcement band 1271 furthest from the skin surface is the third reinforcement band 1271. The upper surface 1272 of the first reinforcement band 1269 is frangibly connected to the lower surface 1276 of the second reinforcement band 1270. The upper surface 1273 of the second reinforcement band 1270 is frangibly connected to the lower surface 1277 of the third reinforcement band 1271. In embodiments comprising a different number of reinforcement bands 1251, the upper surface 1255 of the nth reinforcement band 1251 is coupled to the lower surface 1254 of the n+1^{th} reinforcement band 1251.

The outer edge 1281 of the first reinforcement band 1269 is configured to overlay the peripheral edge of the adhesive patch 106. In other embodiments, the outer edge 1281 of the first reinforcement band 1269 may be configured to extend beyond the peripheral edge of the adhesive patch 106. The outer edge of the second reinforcement band has a larger radius than the first reinforcement band. The outer edge of the third reinforcement band has a larger radius than the outer edge of the second reinforcement band. That is, the outer edge of the n+1^{th} reinforcement band has a larger radius than the outer edge of the nth reinforcement band such that, in use, the outer edge of the nth reinforcement band is hidden by the upper surface of the n+1^{th} reinforcement band.

In use, if the outer edge 1283 of the uppermost reinforcement band 1251 begins to peel away from the skin surface, the user can simply break the frangible connection between the third and second reinforcement bands and remove the third reinforcement band from the skin surface. This means that the peeling cannot spread to the second reinforcement band and thus ensures that the peripheral edge of the adhesive patch is well adhered to the skin surface. Similarly, if the outer edge 1282 of the second reinforcement band 1270 subsequently begins to peel away from the skin surface, the user can simply break the frangible connection between the second and first reinforcement bands and remove the second reinforcement band from the skin surface.

### Non-Isotropic Patches

As outlined above, in some aspects of the present disclosure an adhesive patch (or adhesive patch assembly 1050) may be provided in a first configuration as an on-body device patch (e.g., patch 106) or in a second configuration as an overpatch. Both of these configurations are now described as being capable of having one, two, or more layers exhibiting a non-isotropic characteristic, which has been found to increase the wear time when used with an on-body device. A layer exhibiting a non-isotropic characteristic is referred to herein as a non-isotropic layer. Similarly, an adhesive patch (or adhesive patch assembly), whether an on-body device patch and/or an overpatch, having at least one non-isotropic layer is referred to herein as a non-isotropic patch 2200 (or non-isotropic patch assembly 2250). Each non-isotropic layer may be a backing layer 1051 of the patch.

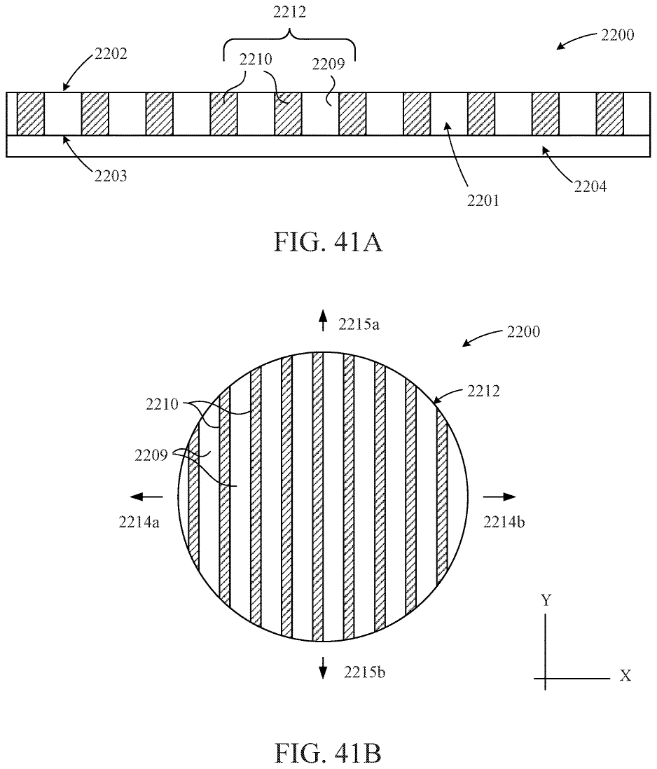

FIG. 41A shows a side view of a non-isotropic patch 2200 that can be configured for use as an on-body device patch or as an overpatch by selection of the appropriate size, material, and placement of any requisite apertures, such as to accommodate a partially implantable sensor or the on-body device housing as the case may be. Here, non-isotropic patch 2200 includes a non-isotropic backing layer 2201 having an upper surface 2202 and a lower surface 2203, where the lower surface is adhered to an adhesive layer 2204 that is configured to adhere to the skin of a wearer when patch 2200 is in use. An additional layer of adhesive (not shown) can be located on a portion or all of upper surface 2202 for adhering to an on-body device housing.

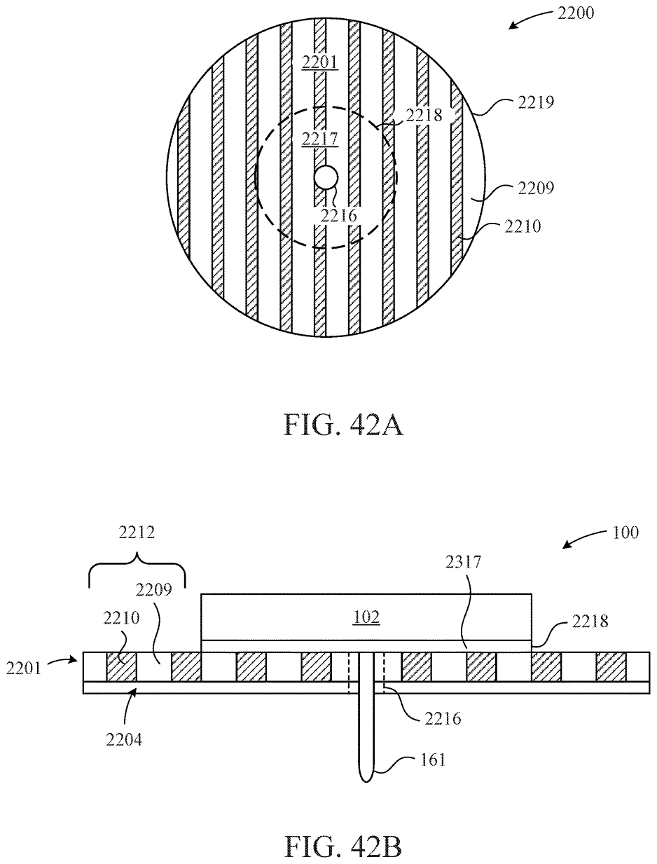

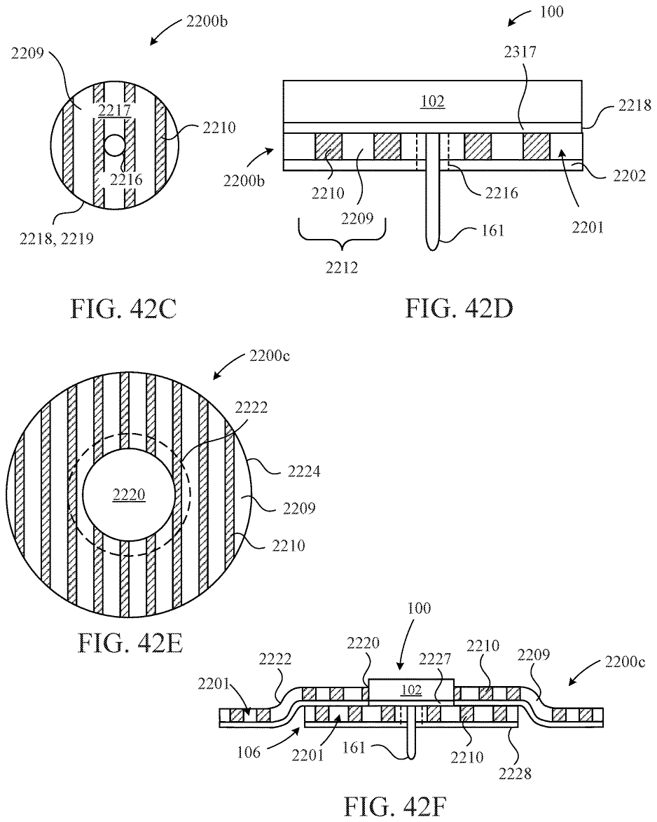

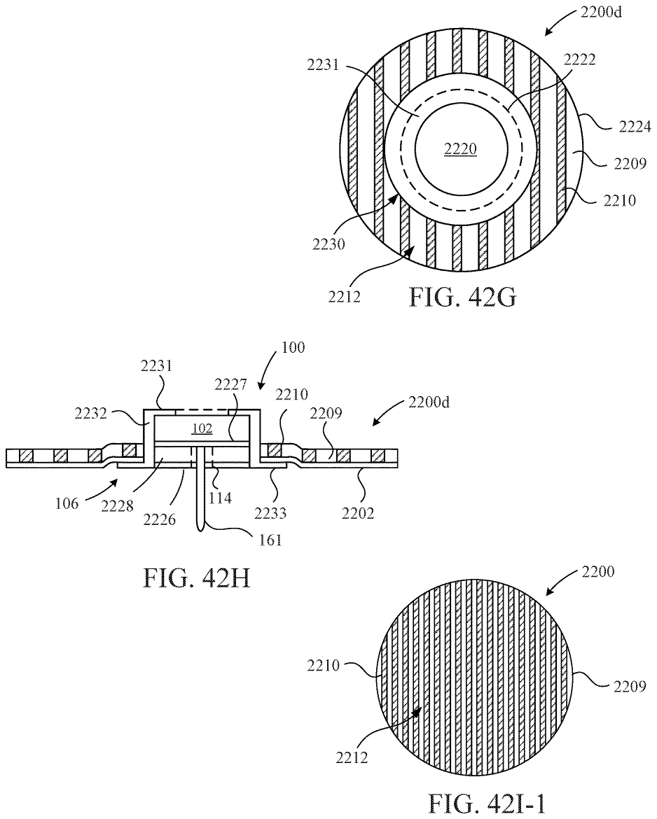

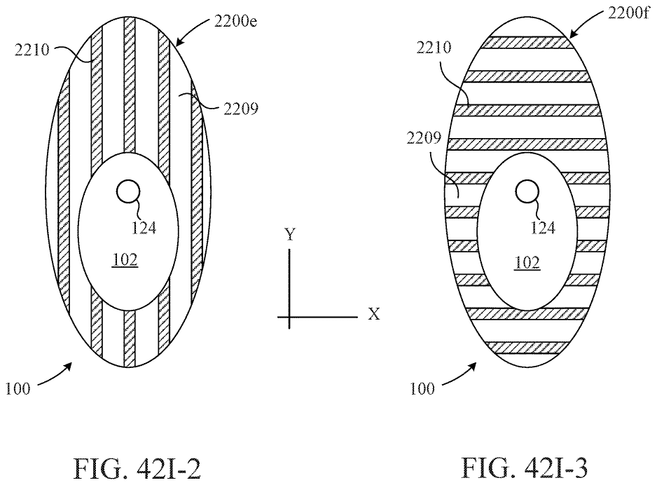

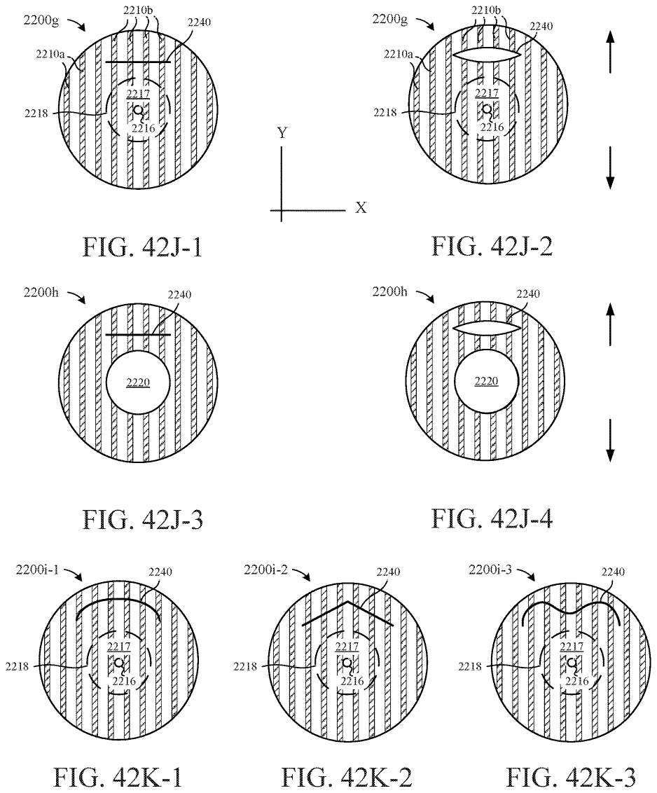

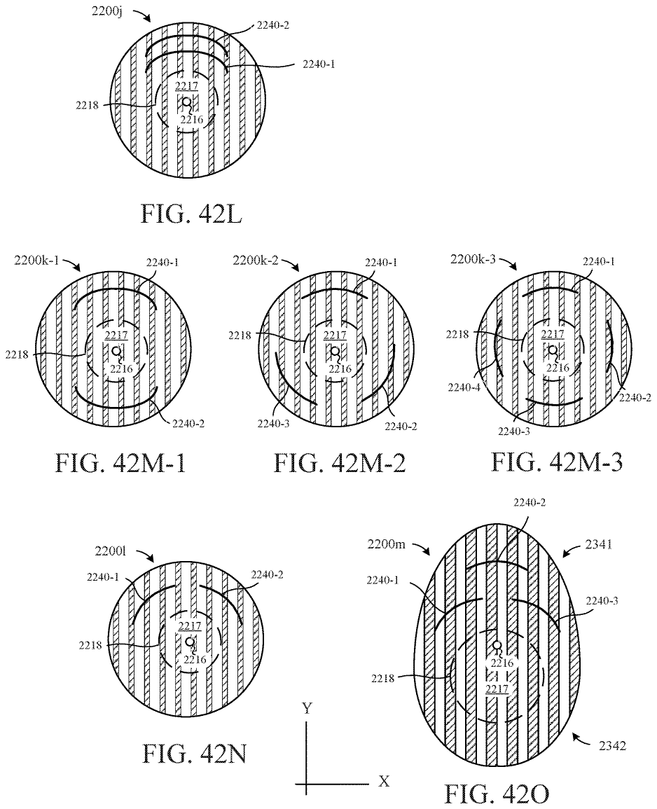

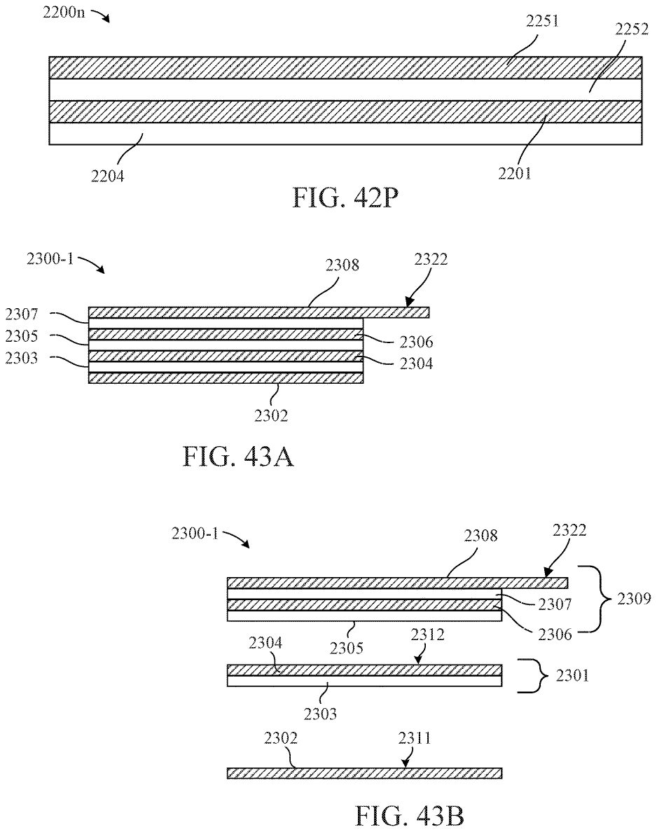

FIG. 41B shows a top down view of upper surface 2202 of non-isotropic layer 2201 of patch 2200. Layer 2201 includes multiple elongate tensile resistant regions 2210 and a main body 2212. Elongate regions 2210 are oriented parallel to the Y axis and perpendicular to the X axis as shown. Layer 2201 is deformable and moves slightly in response to an applied tensile force as indicated by arrows 2214a, 2214b oriented along the X axis as shown. Layer 2201 moves to a lesser extent in response to an applied tensile force indicated by relatively smaller arrows 2215a, 2215b oriented along the Y axis as shown. The distance between regions 2210 can vary, and regions 2201 can be spaced apart or in close proximity (such that the width of region 2210 is greater than the distance between regions 2210). FIG. 42I-1 shows a top down view of an example embodiment of a non-isotropic patch 2200 with a relatively smaller pitch between regions 2209 and 2210, which can be used in all embodiments of non-isotropic layers and patches herein.

The orientation of elongate regions 2210 can vary based on the shape of on body device 100. FIG. 42I-2 shows a top down view of an example embodiment of an on-body device 100 with a non-isotropic patch 2200e and on-body device housing 102 that both have a lateral dimension (e.g., length) along the Y-axis that is relatively larger than the lateral dimension (e.g., width) along the X-axis. In this embodiment elongate regions 2210 are parallel to the Y-axis and the long dimension of patch 2200e and housing 102. FIG. 42I-3 shows a top down view of an example embodiment of an on-body device 100 with a non-isotropic patch 2200f and on-body device housing 102 that both have a lateral dimension (e.g., length) along the Y-axis that is relatively larger than the lateral dimension (e.g., width) along the X-axis. In this embodiment elongate regions 2210 are perpendicular to the Y-axis and parallel to the X axis (the shorter lateral dimension) of patch 2200f and housing 102. Similar configurations can be implemented in embodiments of overpatches having non-uniform length and width.

In some embodiments and depending on the material selected, layer 2201 is generally non-elastic and exhibits minimal elastic return from deformation along either of the X and Y axes. Deformation along the Y axis is more limited by the presence of elongate regions 2210, which resist the tensile force, and which act to maintain the structural integrity of layer 2201 as a whole while worn on the body and subjected to varying environmental and biological conditions. The difference in deformation in response to the same force applied either along the X axis or the Y axis can be 2% or greater in some embodiments, 5% or greater in some embodiments, or 20% or greater in some embodiments.

Main body 2212 can be configured as a sheet, layer, or planar multi-layer matrix and composed of a moisture permeable material and can be non-woven or woven. In some embodiments, main body 2212 is a non-woven spunlace matrix of fibers. Main body 2212 can be a polymeric or plastic material such as, but not limited to polyesters such as polyethylene terephthalate (PET), polyurethanes (PU), polyethylenes (PE), polyethers, elastanes, polyamides such as nylon, and combinations thereof. Main body 2212 can have substantial elasticity or can be substantially inelastic. Elongate regions 2210 can include as a thread, fiber, monolithic strand, and the like extending uni-directionally (or substantially uni-directionally). Elongate regions 2210 can be composed of the same or similar materials as main body 2212. In some embodiments, elongate regions 2210 are relatively thicker, have relatively more fibers per unit area, and/or have relatively greater density of fibers than adjacent regions 2209. Elongate regions 2210 can be at least partially or fully embedded or encapsulated in main body 2212, woven into main body 2212, or formed in integral fashion with main body 2212, such as relatively thicker portions (e.g., ribs) of main body 2212. Elongate regions 2210 can be formed from different materials as well, such as carbon and/or carbon composites. In this embodiment, elongate regions 2210 each have a longitudinal axis oriented parallel to the Y axis and perpendicular to the X axis such that elongate members 2210 extend only along the Y axis and not along the X axis and cannot resist tension applied along the X axis.

FIG. 42A shows a top down view of non-isotropic patch 2200a configured for use as an on-body device patch according to an example embodiment. Here, patch 2200a includes an aperture 2216 for passage of a partially implantable sensor and a sharp therethrough. In this and the other embodiments herein patch 2200a could be configured without aperture 2216 in embodiments where the on-body device has a fully implantable or wholly ex vivo sensor. Adhesive layer 2217 is located on upper surface 2202 and has an outer periphery 2218 that is generally commensurate in size and shape to the size and shape of the on-body device housing (not shown). Patch 2200a has an outer periphery 2219 that is larger (e.g., in width or diameter) than periphery 2218 such that a portion of layer 2201 will extend beyond the periphery of the on-body device housing when coupled together. FIG. 42B shows a side view of an embodiment of on-body device 100 having this embodiment of non-isotropic patch 2200a adhered with on-body device housing 102 via adhesive layer 2217 and with sensor 161 extending through aperture 2216.

FIG. 42C shows a top down view of a non-isotropic patch 2200b configured for use as an on-body device patch according to another example embodiment. Here, patch 2200b again includes aperture 2216 for passage of a partially implantable sensor and a sharp therethrough (if configured for such). Adhesive layer 2217 is located on upper surface 2202 and has an outer periphery 2218 that is generally commensurate in size and shape to the size and shape of the on-body device housing (not shown), which in this embodiment is also commensurate with outer periphery 2219 of layer 2201 such that layer 2201 will generally terminate at the periphery of the on-body device housing when coupled together. In practice, outer periphery 2219 may be slightly smaller in width or diameter (e.g., by one or a few millimeters, or within 5% or 10% of overall width) than that of the outer periphery of the on-body device housing such that patch 2200b does not extend all the way to the on-body device housing outer periphery. Alternatively, outer periphery 2219 may be slightly larger in width or diameter (e.g., by one or a few millimeters, or within 5% or 10% of overall width) such that patch 2200b extends just beyond the on-body device housing outer periphery. FIG. 42D shows a side view of an embodiment of on-body device 100 having this embodiment of non-isotropic patch 2200b adhered with on-body device housing 102 via adhesive layer 2217 and with sensor 161 extending through aperture 2216.

FIG. 42E shows a top down view of a non-isotropic patch 2200c configured for use as an overpatch according to another example embodiment. Here, patch 2200c includes aperture 2220 for passage of an on-body device housing (not shown) therethrough. In this and the other embodiments herein the location of aperture 2220 can be oriented in the center of overpatch 2200 or can be offset from the center. In this and the other embodiments herein the shape of aperture 2220 can match that of the on-body device housing (e.g., both being circular, elliptical or otherwise, such as the on-body device housing embodiments of FIGS. 1N-3 through 1N-20). The width or diameter of aperture 2220 is generally slightly larger than the width or diameter of the on-body device housing to facilitate passage therethrough. This embodiment of patch 2200c is suited for placement over an on-body device having a patch 106 that is of larger width or diameter than the on-body device housing. Dashed line 2222 indicates an example outer periphery of patch 106 of the on-body device, and outer periphery 2224 of patch 2200c is substantially larger in width of diameter than periphery 2222. FIG. 42F shows a side view of this embodiment of non-isotropic patch 2200c adhered over an embodiment of on-body device 100. In this embodiment, on-body device 100 includes a patch 106 that has an isotropic backing layer 2226, an upper adhesive layer 2227 and a lower adhesive layer 2228.

FIG. 42G shows a top down view of a non-isotropic patch 2200d configured for use as an overpatch according to another example embodiment. Here, patch 2200d includes aperture 2220 for passage of an on-body device housing (not shown) therethrough. Patch 2200d also includes a support structure 2230. Support structure 2230 can be configured to receive on-body device housing (not shown) as explained more with respect to FIG. 42H. While not limited to such, this embodiment of patch 2200d is suited for placement over an on-body device having a patch 106 that is of generally the same or smaller width or diameter than the on-body device housing (see, e.g., the embodiment described with respect to FIGS. 42C and 42D). Dashed line 2222 indicates an example outer periphery of patch 106 of the on-body device, and outer periphery 2224 of patch 2200d is again substantially larger in width of diameter than periphery 2222.

FIG. 42H shows a side view of this embodiment of non-isotropic patch 2200d adhered over an embodiment of on-body device 100. Support structure 2230 is shown in more detail and can include an upper retainer structure 2231, a sidewall 2232, and a lower retainer structure 2233. Support structure 2230 includes a recess in which on-body device housing 102 can be received. Here, retainer structure 2231 is configured to extend radially inwards from sidewall 2232 over at least a portion of the upper surface of on-body device housing 102. In other embodiments retainer structure 2231 can extend over more or the entirety of the upper surface of housing 102. Retainer structure 2231 can act to retain housing 102 on the body of the wearer, though the inclusion of retainer structure 2231 is not necessary in all embodiments as patch 2200d as a whole also retains on-body device 100 on the wearer. Retainer structure 2233 can extend radially outwards from sidewall 2232 underneath adhesive layer 2202 of patch 2200d such that adhesive layer 2202 adheres structure 2230 to layer 2201 of patch 2200d. Other attachment techniques can be used in other embodiments, such as welding, different adhesives, weaving layer 2201 to structure 2230, and others.

Non-Isotropic Patches with Openings

Non-isotropic patches exhibit beneficial degrees of breathability and durability making them suitable for use on human bodies for extended wear durations. In some cases, non-isotropic patches can be prone to exhibit peel away, e.g., around the peripheral edge, while being worn on the skin. Movement of the body in a manner that exhibits repeated tensile force on the non-isotropic patch along the axis of minimal deformation, e.g., the axis parallel to the elongate tensile resistant regions along which deformation is restricted by the elongate tensile resistant regions, can lead to separation of the adhesive and subsequently peel away of the patch from the skin or other underlying object. Placement of one or more openings in the non-isotropic patch in locations that traverse and segment one or more of the elongate tensile resistant regions can increase the deformability of the non-isotropic patch along this restricted axis.

FIG. 42J-1 shows a top down view of a non-isotropic patch 2200g having an opening 2240 that traverses and segments four elongate regions 2210b while leaving the remaining segments (such as 2210a) intact. The number of elongate regions segmented by opening 2240 is dependent on the length of opening 2240, the width of regions 2210 and the size of the spacing between regions 2210. FIG. 42J-1 shows patch 2200g in an at-rest state where opening 2240 is closed and FIG. 42J-2 shows patch 2200g in a deformed state where opening 2240 has opened in response to tensile forces pulling along the Y axis. In this embodiment, patch 2200g is configured as a main patch for an on-body device housing and includes a sensor aperture 2216 and upper adhesive layer 2217 with outer periphery 2218 that is generally commensurate in size and shape to the size and shape of the on-body device housing (not shown). The embodiments of patches 2200 having an opening 2240 can be configured for use as a main patch of an on-body device such as shown in FIGS. 42J-1 and 42J-2, or as an overpatch 2200h such as shown in FIGS. 42J-3 and 42J-4 (with opening 2240 shown in closed and open states respectively).

Opening 2240 can be configured as a narrow opening such as a slit or cut in patch 2200. The opening 2240 can be cut, sliced, etched, or ablated into the patch such as with a mechanical blade or high energy beam or laser. The narrow opening can be formed without removing an area of material from patch 2200, or by removing only minimal area in the shape of a short (flattened) and wide rectangle, short (flattened) and wide oval, a thin curved region, or the like, which would minimize material removed.

For ease of illustration, opening 2240 will be shown and described as a slit although the other aforementioned configurations can be used in each case. Further, patch 2200 will be shown and described as a main patch for an on-body device, though patch 2200 can similarly be configured as an overpatch in each embodiment and peripheral line 2218 can be considered as approximating the location where central opening 2220 would be.

In the embodiments described herein, each opening 2240 can have a straight shape when at-rest (not subjected to tensile force) such as shown in FIG. 42J-1 and 42J-3. In the embodiments described herein, each opening 2240 can have a curved shape when at rest such as shown in patch 2200i-1 of FIG. 42K-1. In the embodiments described herein, each opening 2240 can have a sawtooth or V-shape when at rest such as shown in patch 22001-2 of FIG. 42K-2. In the embodiments described herein, each opening 2240 can have a wave shape when at rest such as shown in patch 2200i-3 of FIG. 42K-3.

In the embodiments described herein, each opening 2240 can be at the same radial location (or orientation) about a central point of the patch 2200. FIG. 42L shows an embodiment of patch 2200j having a first opening 2240-1 located relatively closer to a central point (e.g., in sensor opening 2216) than a second opening 2240-2. The openings 2240-1 and 2240-2 are aligned (e.g., stacked) along a radial location at approximately zero degrees (e.g., north). Any number of two or more openings can be stacked as shown at any one radial orientation with more than one stack at different radial locations.

In the embodiments described herein, each opening can be at a different radial location about a central point of the patch 2200. FIG. 42M-1 shows an embodiment of patch 2200k-1 having a first opening 2240-1 on a first side of patch 2200k-1 and a second opening 2240-2 on a second side of patch 2200k-2. Opening 2240-1 is aligned at a first radial location at approximately zero degrees (e.g., north) and opening 2240-2 is aligned at a second radial location at approximately 180 degrees (e.g., south). FIG. 42M-2 shows an embodiment of patch 2200k-2 having a first opening 2240-1 aligned at a first radial location at approximately zero degrees, a second opening 2240-2 aligned at a second radial location at approximately 120 degrees, and a third opening 2240-3 aligned at a third radial location at approximately 240 degrees. FIG. 42M-3 shows an embodiment of patch 2200k-3 having a first opening 2240-1 aligned at a first radial location at approximately zero degrees, a second opening 2240-2 aligned at a second radial location at approximately 90 degrees, a third opening 2240-3 aligned at a third radial location at approximately 180 degrees, and a fourth opening 2240-4 aligned at a fourth radial location at approximately 270 degrees. In the embodiments of FIGS. 42M-1, 42M-2, and 42M-3 the openings 2240 are arranged at the same interval (180 degrees, 120 degrees, and 90 degrees respectively) to provide a symmetrical radial arrangement.

In the embodiments herein, the openings 2240 can also be arranged radially asymmetrically with respect to the central point. FIG. 42N shows a patch 22001 with a first opening 2240-1 and a second opening 2240-2 located on the same side of patch 22001. The first opening 2240-1 is at approximately 315 degrees (with zero degrees being north) and the second opening 2240-2 is at approximately 45 degrees.

In the embodiments herein, the openings 2240 can also radially overlap. FIG. 42O shows a patch 2200m with a first opening 2240-1 at approximately 315 degrees (with zero degrees being north), a second opening 2240-2 at approximately 0 degrees, and a third opening at approximately 45 degrees with opening 2240-2 partially radially overlapping with openings 2240-1 and 2240-3. In this embodiment, the openings 2240 are also located on the same side 2241 of the patch 2200m, which has the adhesive layer 2217 for the on-body device housing offset from the central point in sensor aperture 2216 so that the housing will located generally on the side 2242 opposite to side 2241. The sides 2241 are here referenced as different locations on the Y axis, but are not limited to such. Offsetting the on-body device housing to one side of the adhesive patch can free more surface area for placement of openings 2240 on the opposite side of the patch.

The one or more openings 2240 that traverse and segment one or more elongate regions 2210 are distinct from other openings present in the patch. These other openings include any one or more sensor openings 2216 in a patch 2200 configured for use as a main body patch of an on body device and the on-body device housing (and/or support structure) opening 2220 present in a patch 2200 configured as an overpatch. For example, sensor opening 2216 is relatively small so as not to remove patch surface area that would otherwise assist in adhering the on-body device 100 to the skin. Sensor opening 2216 also may not traverse or segment any regions 2210, and if it does then the effect will be minimal given the size of sensor opening 2216. Further, sensor opening 2216 is typically formed by removal of a region of patch material, e.g., in the shape of a circular or elliptical hole, which does not provide the same capability of opening and closing as the openings 2240 can be configured to provide in various slit and similar embodiments. Still further, sensor opening 2216 is often located in a central location in the patch with the on-body device housing adhered around the opening 2216, restricting the amount of deformation that opening 2216 might provide. There may be multiple sensor openings 2216 in a patch, such as for multiple sensors measuring different analytes, and the same or similar reasoning applies to each. The housing (or support structure) opening 2220 is similarly formed by removal of a region of patch material, e.g., in the shape of a circular or elliptical hole, which does not provide the same capability of opening and closing as the openings 2240 can be configured to provide in various embodiments. The housing opening 2220 is also typically configured to maintain a close and contacting fit for either the support structure 2230 or the on-body device housing therein which limits the amount of deformation that opening 2216 might provide.

Non-Isotropic Patches with Polymeric Layers

As an alternative to openings 2240, or in addition to openings 2240, a patch having a non-isotropic layer can include a polymeric layer to improve the wear performance. FIG. 42P shows a side view of a non-isotropic patch 22001. In this embodiment, patch 22001 includes a polymeric layer 2251 adhered to an upper surface 2253 of the non-isotropic layer 2201 by an adhesive layer 2252. In other embodiments the locations of the non-isotropic layer 2201 and polymeric layer 2251 can be reversed such that the non-isotropic layer 2201 is above the polymeric layer 2251. The polymeric layer 2251 can be or include a polymeric or plastic material such as, but not limited to polyesters such as polyethylene terephthalate (PET), polyurethanes (PU), polyethylenes (PE), polyethers, elastanes, polyamides such as nylon, and combinations thereof. In some embodiments the polymeric layer includes polyester, polyurethane, or a combination of both. In many embodiments the polymeric layer 2251 can have relatively greater elasticity than the non-isotropic layer 2201. The added elasticity can assist patch 2200 in returning to an at-rest state after deformation and this can improve the wear duration of the patch 2200. Patch 2200 having a non-isotropic layer 2201 and a polymeric layer 2251 can also have additional layers for additional functionality. In these embodiments the MVTR of patch 2200 is sufficient to allow significant moisture to pass therethrough, and can be for example greater than 115 grams/square meter/day.

The embodiments of patches described in this section and/or with respect to any and all of FIGS. 41A-42P can be used in combination with any and all of the on-body devices, reinforcement devices, patches, analyte monitoring systems, and/or methods described in the preceding sections and/or with respect to FIGS. 1-40B and FIGS. 43A-46G.

### Overpatch Assemblies

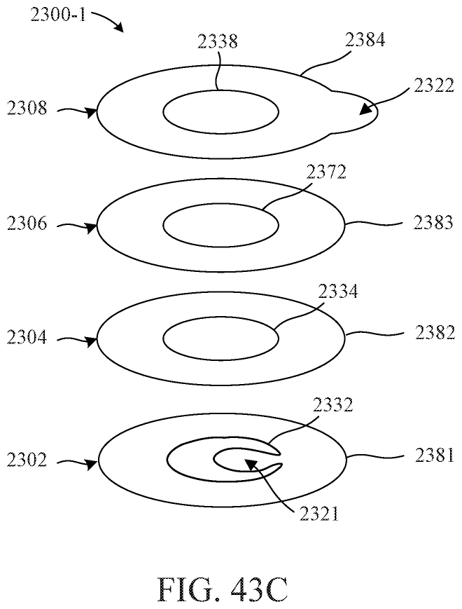

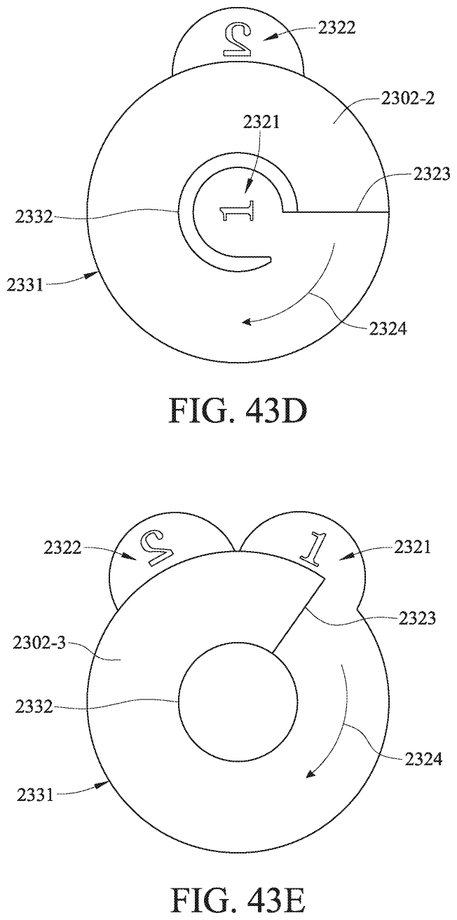

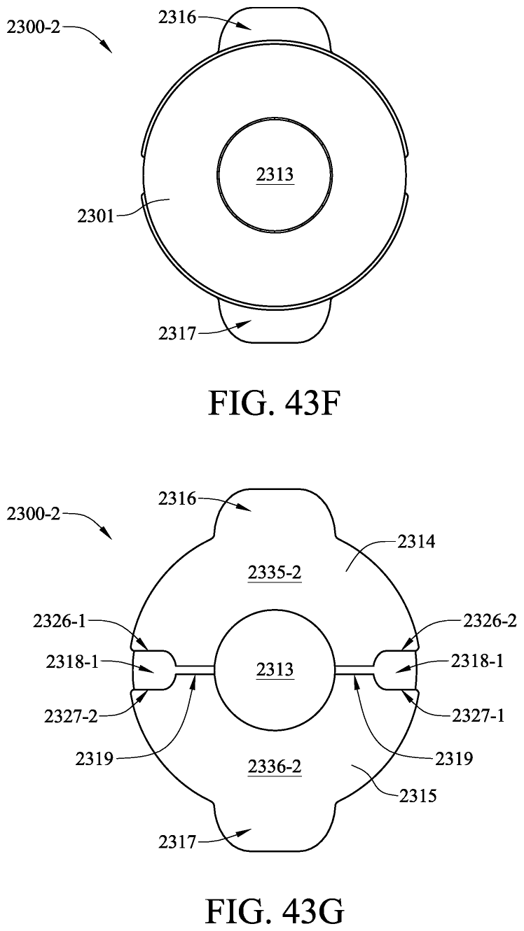

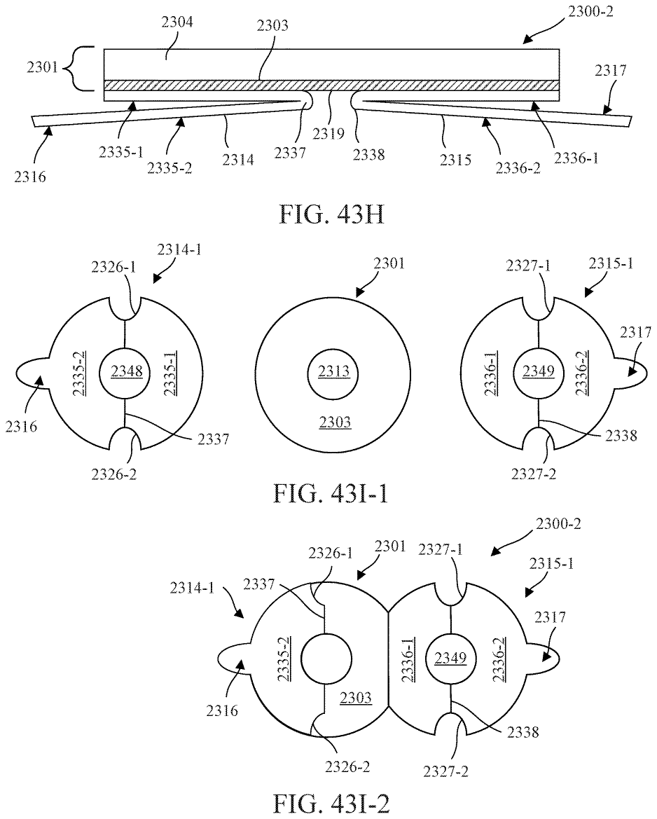

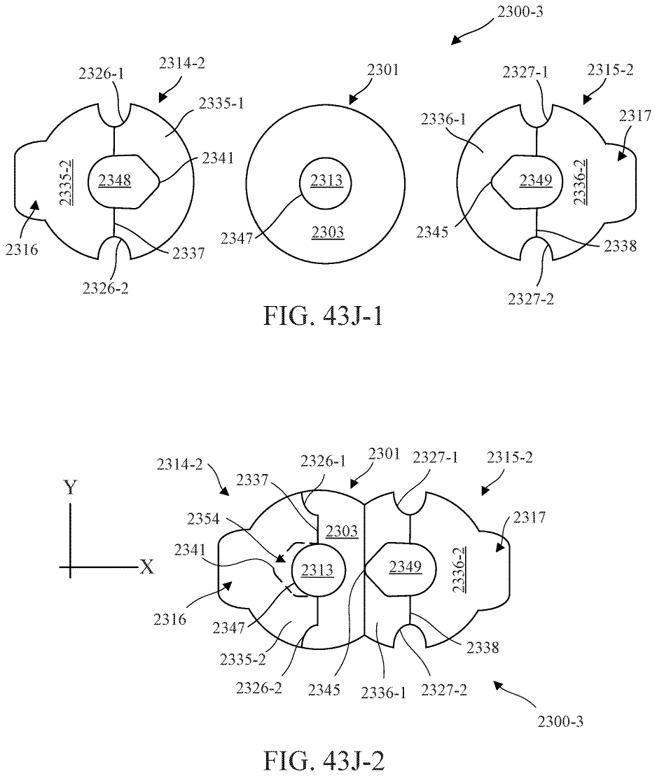

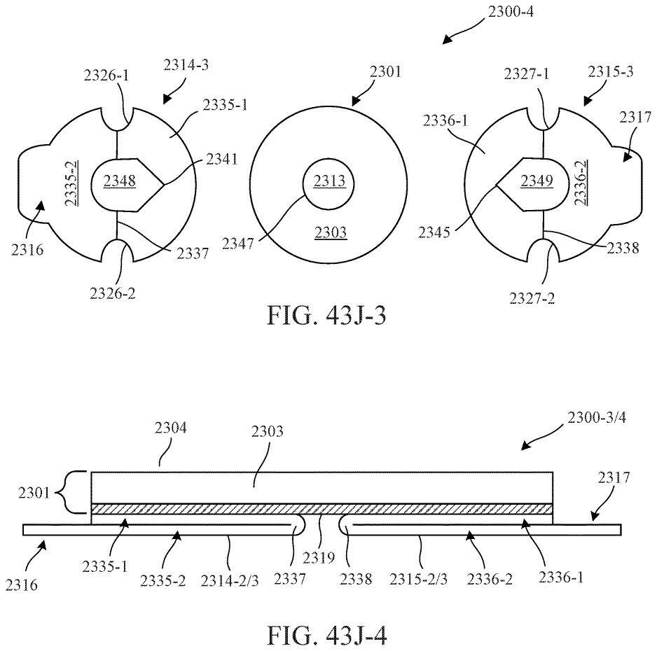

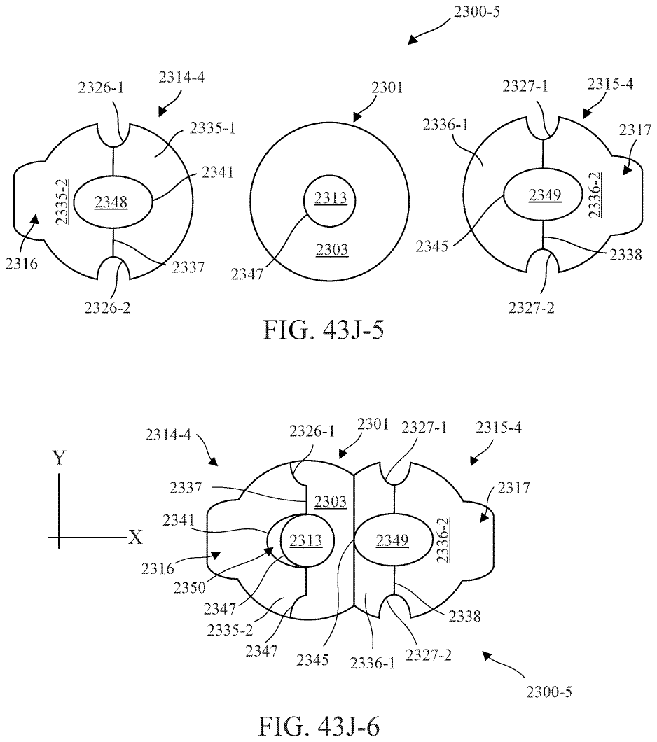

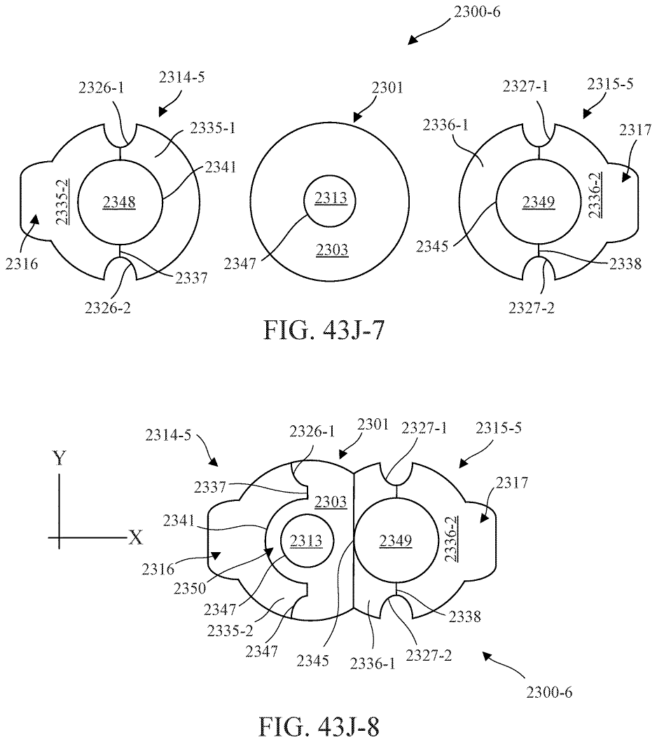

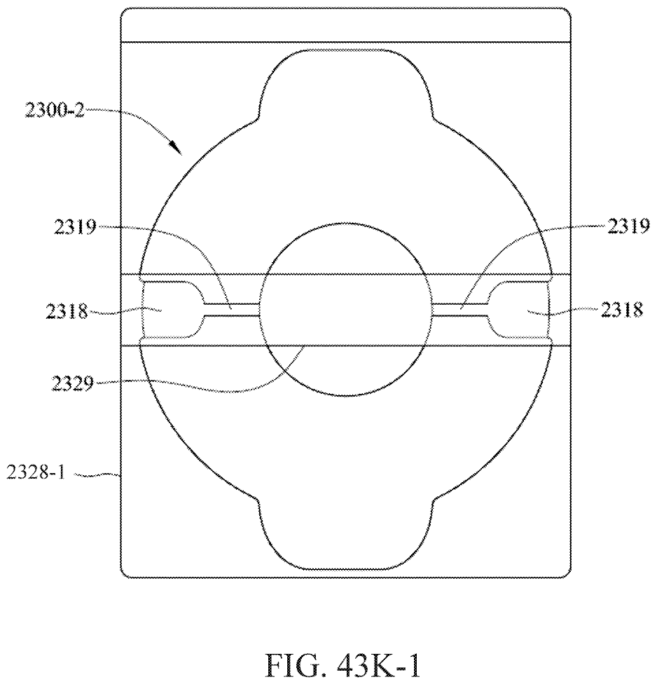

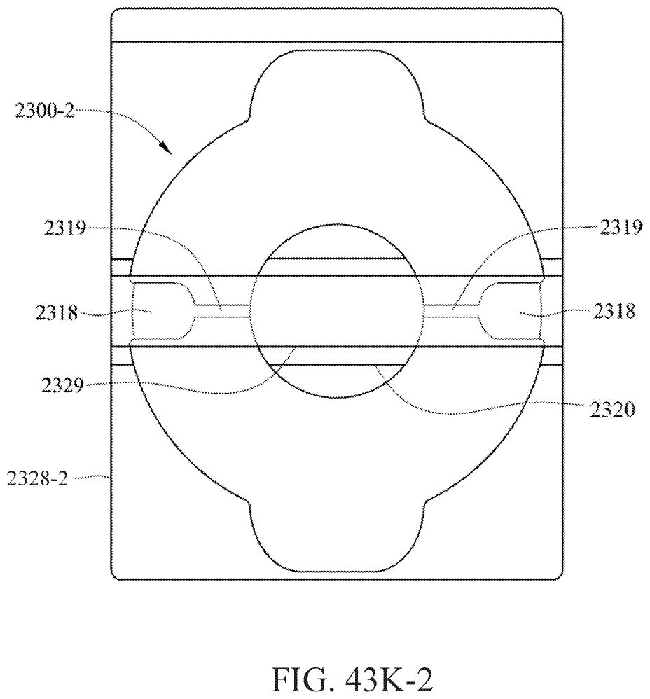

Described herein are overpatch assemblies with features that improve ease of application and/or wear duration. FIG. 43A shows a side view of an overpatch assembly 2300-1 for an on-body device according to an example embodiment. FIG. 43B shows an unassembled side view of overpatch assembly 2300-1. In FIGS. 43A-43B the height of overpatch assembly 2300-1 is exaggerated in relation to the width to facilitate depiction of the various layers of construction. FIG. 43C shows an elevated perspective view of exploded overpatch assembly 2300-1 and FIG. 43D shows a bottom up view of overpatch assembly 2300-1 in a fully assembled state. The terms upper and lower as used in the description of this embodiment are with reference to the skin of the wearer in the location where the overpatch is deployed, with the terms “lower,” “underlying”, and “bottom” referring to entities that are relatively closer to the skin and the terms “upper” and “top” referring to entities that are relatively farther from the skin. Overpatch assembly 2300-1 can be configured such that the deployed overpatch within assembly 2300-1 can have the features of any and all of the other embodiments of overpatches described herein.

In this embodiment assembly 2300-1 includes four structural layers: lower liner 2302, backing layer 2304, stiffener layer 2306, and upper liner 2308. In this embodiment assembly 2300-1 includes three adhesive layers: skin adhesive layer 2303, stiffener adhesive layer 2305, and upper liner adhesive layer 2307, although more or less than these layers can be implemented in practice. Backing layer 2304 and skin adhesive layer 2303 together form overpatch 2301 configured for extended wear on the wearer's skin, with the other layers 2302, 2305, 2306, 2307, and 2308 being configured for removal during application of the overpatch. Although each of structural layers 2302, 2304, 2306, 2308 are shown here as one layer, each can include multiple layers configured as a laminate or coated structural entity.

Each of the structural layers 2302, 2304, 2306, and 2308 has an outer periphery 2381, 2382, 2383, and 2384, respectively, that generally matches each other in size and shape. Similarly, each of the structural layers 2302, 2304, 2306, and 2308 has an inner peripheral edge 2332, 2334, 2372, and 2338, respectively, that define apertures, and that generally match each other in size and shape and is configured to match the shape and be slightly larger than the outer periphery of the on-body device housing (not shown) such that the on-body device housing can be received through layers 2303-2308. The outer periphery and shape of overpatch 2301 can be selected in any desired size and shape as described herein to facilitate reaching the desired wear duration.

To apply overpatch 2301, a user removes lower liner 2302 from skin adhesive layer 2303 to expose the skin adhesive of layer 2303. The user can remover lower liner 2302 by grasping tab portion 2321 and manually pulling to gradually separate lower liner 2302 from backing layer 2304. The user can then apply the remaining layers of assembly 2300-1 (together as one unit) over the on-body device 100 already present on the skin. Stiffener layer 2306 can have a rigidity that is relatively greater than backing layer 2304 such that assembly 2300-1 substantially retains its annular form during the application process. This reduces the likelihood that the overpatch assembly 2300-1 will bend, droop, or collapse during application and thus reduces the likelihood that overpatch assembly will be adhered to the user's skin in an unintended manner. Without stiffener layer 2306, overpatch assembly 2300-1 could be applied to the skin in a manner misaligned with on-body device housing, thus requiring the user to remove and reapply assembly 2300-1 which can be uncomfortable and can reduce the effectiveness of the skin adhesive over the manufacturer's intended wear duration.

Once assembly 2300-1 is applied and adhered in the intended location with on-body device housing received through apertures defined by edges 2334, 2372, and 2338, the user can peel off and remove stiffener layer 2306 and upper liner 2308 together as one upper unit 2309 (see FIG. 43B) by grasping tab portion 2322 and manually pulling to gradually separate and remove upper liner 2308 and stiffener layer 2306 together from backing layer 2304. The user can secure overpatch 2301 by manually pressing the surface exposed to the user (e.g., backing layer 2304 or stiffener unit 2309) to improve adhesion of layer 2303 with the skin. Removal of upper unit 2309 leaves overpatch 2301 adhered to the skin of the on-body device wearer and the portion of the on-body device patch 106 extending beyond the outer periphery of the on-body device housing. The user can dispose of lower liner 2302 and upper unit 2309.

Assembly 2300-1 is configured such that the degree of adhesion (e.g., peel adhesion) between the adhesive layers and the structural layers those adhesive layers are in contact with has a hierarchy of relative strengths that permits assembly 2300-1 to be unassembled (e.g., separated) in the proper order. This can be accomplished by using materials on adjacent layers that have different tendencies to adhere to the adhesive therebetween, by using different layer constructions (e.g., smooth as compared to textured) having different tendencies to adhere, by using different adhesives with varying degrees of bond strength, and/or a combination of any two or all three of the aforementioned. Hydrophobic materials tend to exhibit lesser degrees of adhesion than hydrophilic materials. Textured materials such as roughened surfaces, mesh materials, non-woven spunlace materials, and/or woven materials will have a greater tendency to adhere than materials with lower surface friction (e.g., smooth). Coatings of readily releasable material, such as silicone or another an anti-stick coating can reduce the degree of adhesion of that coated layer to an adjacent adhesive.

To permit separation of lower liner 2302 first, the degree of adherence (e.g., peel adhesion) of adhesive layer 2303 to lower liner 2302 is relatively less than that of adhesive layer 2303 to backing layer 2304, adhesive layer 2305 to backing layer 2304 and stiffener layer 2306, and adhesive layer 2307 to stiffener layer 2306 and upper liner 2308. Lower liner 2302 will thus separate from backing layer 2304 at a lower pull force threshold than stiffener layer 2306 separates from upper liner 2308 and than stiffener layer 2306 separates from backing layer 2304. The upper surface 2311 of lower liner 2302 can be coated with a readily releasable material (e.g., a nonstick material) such as silicone to facilitate removal. In addition or alternatively, lower liner 2302 can be composed of a readily releasable material that adheres to adhesive layer 2303 to a lesser degree than backing layer 2304.

To permit upper liner 2308 and stiffener layer 2306 to be removed together as one upper unit 2309, the degree of adherence of adhesive layer 2307 to the lower side of upper liner 2308 and the upper side of stiffener 2306 is relatively greater than that of adhesive layer 2305 to at least one of the lower side of stiffener layer 2306 and/or the upper side of backing layer 2304. Stiffener layer 2306 will thus separate from backing layer 2304 at a lower pull force threshold than stiffener layer 2306 separates from upper liner 2308. The upper surface 2312 of backing layer 2304 can be coated or lined with a readily releasable material (e.g., a nonstick material) such as silicone to facilitate removal.

To permit upper unit 2309 to be removed without separating skin adhesive layer 2303 from the user's skin, the degree of adherence (e.g., peel adhesion) of adhesive layer 2303 to skin and to the lower side of backing layer 2304 is relatively greater than that of adhesive layer 2305 to the lower side of stiffener layer 2306 and the upper side of backing layer 2304. Stiffener layer 2306 will thus separate from backing layer 2304 at a lower pull force threshold than backing layer 2304 separates from the skin but at a higher pull force threshold than backing layer 2304 separates from lower liner 2302. Thus, in this embodiment overpatch 2300-1 is configured such that first adhesive layer 2305 has a lower degree of adherence to those structural layers it is contact with above and below, as compared to the degree of adherence that second adhesive layer 2303 has with skin and with the structural layer above it. Stiffener layer 2306 can itself be configured with a pull tab portion 2322 or similar graspable feature in other embodiments, and in those embodiments upper liner 2308 and adhesive layer 2307 can be omitted. However, if upper liner 2308 and adhesive layer 2307 are present, then overpatch 2300-1 is configured such that first adhesive layer 2305 has a lower degree of adherence to stiffener layer 2306 and/or backing layer 2304 than the degree of adherence that third adhesive layer 2307 has with stiffener layer 2306 and upper liner 2308.

To the extent other structural layers and/or adhesive layers are present in assembly 2300-1, the degree of adhesion can be set so that those layers peel away at the appropriate time in the sequence of unassembly, or so that they do not peel away at all when handled by the user.

Although adhesive layer 2303 is depicted as attached to backing layer 2304, in practice a relatively small amount of adhesive 2303 may also be present on lower liner 2302 after separation. Similarly, adhesive layer 2305 is depicted as attached to stiffener layer 2306, but in practice some of adhesive 2305 will also be present on backing layer 2304 after separation. In some embodiments, the adhesive layers 2303, 2305, and 2307 (and others) may include an acrylic adhesive such as a medical extended wear acrylic adhesive (e.g., manufactured by 3M). The adhesive layers may include other adhesives such as silicone adhesives, polyisobutylene and other hydrocarbon adhesives, polyurethane adhesives, natural rubber adhesives, synthetic rubber adhesives, and the like.

FIG. 43D shows a bottom up view of overpatch assembly 2300-1 prior to application. As can be seen here, pull tab portion 2321 of liner 2302 is located in a central space of assembly 2300-1 where the on-body device housing will be received. Portion 2321 is also labelled with an indicium indicating the order in which liner 2302 is to be removed, which in this example is the number one. This indicium can be printed on portion 2321 (on the lower side or on both the lower and upper sides) or can be cut through portion 2321 so as to be visible from both sides. Pull tab portion 2322 of upper liner 2308 is also visible here as it projects past periphery 2381 of lower liner 2302. Pull tab portion 2322 can also have an indicium indicating the order of removal, which in this example is the number two. This indicium can be printed on portion 2322 (on the upper side or on both the lower and upper sides) or can be cut through portion 2322 so as to be visible from both sides. Here, the indicia are both cut through their respective portions, and are oriented for reading from opposite sides (e.g., the indicium on portion 2321 is readable from the bottom side while the indicium on portion 2322 is readable from the upper side of assembly 2300-1). A slit (or cut) 2323 is present in lower liner 2302 on one side of portion 2321 from inner periphery 2332 to outer periphery 2381. This slit 2323 allows for lower liner 2302 to be removed in directed fashion that reduces the pull force required for separation of lower liner 2302 from adhesive 2303 and backing layer 2304. Lower liner 2302 can be removed in direction 2324 (which is clockwise here but can be counterclockwise in other embodiments) that follows the curvature of liner 2302 to form a 360 degree path around assembly 2300-1. Liner 2302 is thus more easily separated than if removal proceed both in clockwise and counterclockwise fashion simultaneously.

FIG. 43E shows a bottom up view of assembly 2300-1 with pull tab portion 2321 instead on the outer periphery 2381 of lower liner 2302 according to another embodiment. Here, slit 2323 extends from a base of pull tab portion 2321 to inner periphery 2332. Again, lower liner 2302 can be configured to be removed in clockwise or counterclockwise fashion with reduced pull force required.

FIGS. 43F and 43G show a top down view and bottom up view, respectively, of another example embodiment of an overpatch assembly 2300-2. In this embodiment overpatch assembly 2300-2 includes an overpatch 2301 including at least a backing layer 2304 and underlying adhesive layer 2303 (not labeled). Overpatch assembly 2300-2 includes an interior opening 2313 through which an on-body device housing can be received. Adhesive layer 2303 is lined by at least one removable liner. In this embodiment adhesive layer 2303 is lined by first liner 2314 having a pull tab portion 2316 and second liner 2316 having a pull tab portion 2317. In other embodiments, three or more removable liners.

FIG. 43H shows a side view of overpatch assembly 2300-2. Here it can be seen that liners 2314 and 2315 are each adhered to adhesive layer 2303 in a folded state. A first portion 2335-1 of liner 2314 is adhered to adhesive layer 2303 of overpatch 2301. Liner 2314 has a fold 2337 where it reverses direction back upon itself, and a second portion 2335-2 of liner 2314 is located beneath portion 2335-1 and unadhered to layer 2303. Similarly, a first portion 2336-1 of liner 2315 is adhered to adhesive layer 2303 of overpatch 2301. Liner 2315 has a fold 2338 where it reverses direction back upon itself, and a second portion 2336-2 of liner 2315 is located beneath portion 2336-1 and unadhered to layer 2303.

FIG. 43I-1 is an exploded view showing overpatch 2301 and liners 2314-1 and 2315-1 in their unassembled and unfolded states. Liner 2314-1 includes a crease indicating where fold 2337 occurs and liner 2315-1 includes a crease indicating where fold 2338 occurs. Liners 2314-1 and 2315-1 can each have one or more partial or full openings or open regions 2326 to allow exposure of adhesive layer 2303. In this embodiment, liner 2314-1 includes a first open slot 2326-1 and a second open slot 2326-2 on opposite sides and aligned with the crease of fold 2337. Similarly, liner 2315-1 includes a first open slot 2327-1 and a second open slot 2327-2 on opposite sides and aligned with the crease of fold 2338. Liners 2314-1 and 2315-1 each have a central opening 2348 and 2349, respectively, defined by inner peripheral edges of liners 2314-1 and 2315-1.

Referring back to FIG. 43G, overpatch assembly 2300-2 can include one or more regions where adhesive layer 2303 is exposed. In this embodiment, a gap 2319 is present between liners 2314 and 2315 exposing adhesive layer 2303 therebetween. This gap is made larger in regions 2318-1 and 2318-2 by the presence of slots 2326 and 2327. Specifically, gap 2319 is made larger in region 2318-1 by folded over slot 2326-1 and folded over slot 2327-1 which are in positions that face (or oppose) each other. Gap 2319 is made larger in region 2318-2 by folded over slot 2326-2 and folded over slot 2327-2 which are in positions that face (or oppose) each other.

The exposure of adhesive layer 2303 in gap region 2319 and regions 2318-1 and 2318-2 provides a sticky surface that a user can use to attach overpatch assembly 2300-2 to the skin of the on-body device wearer (which may the same or a different person). A method of applying overpatch assembly 2300-2 can include positioning interior space 2313 over a housing of an on-body device while a first portion (e.g., regions 2318-1, 2318-2, and 2319) of adhesive layer 2303 of the overpatch 2301 is exposed and a second portion of adhesive layer 2303 is unexposed (e.g., the portion covered by adhered liners 2314 and 2315). The user can then adhere the exposed first portion of the adhesive layer to skin of the wearer while the second portion of the adhesive layer is unexposed. If the overpatch assembly 2300-2 is not initially positioned as desired, or is mispositioned, then the user can reposition the overpatch assembly, such as by un-adhering the first portion, moving the overpatch assembly 2300-2, and then re-adhering the first portion.

When the overpatch assembly is in the desired proper position, the user can remove liners 2314 and 2315 in any desired order, or simultaneously. For example, referring again to FIG. 43I-1, the user can grasp and pull the pull tab portion 2317 of liner 2315-1 while the first portion of the adhesive layer 2303 is adhered to the skin, and also optionally while manually holding the remaining overpatch assembly 2300-2 in position on the skin and over the on-body device. As the user pulls tab portion 2317, liner 2315-1 begins to concurrently unfold or unroll and separate from (be removed from) adhesive layer 2303 beneath overpatch 2300. FIG. 43I-2 shows a bottom view of overpatch assembly in the midst of removal of liner 2315-1. Here it can be seen that liner 2315-1 unrolls in a manner such that the location of fold 2338 progresses across the surface of adhesive layer 2303 and liner 2315-1. This allows liner 2315-1 to be removed while maintaining a low profile that does not require overpatch 2301 to be lifted up and off of the skin. As the user pulls liner 2315-1 off the user can optionally hold and adhere the newly exposed portion of layer 2303 to the underlying skin with his or her hand, increasing the adhered surface area and progressing the application of overpatch 2301. This process can be continued until liner 2315-1 is fully removed and liner 2314-1 is also removed in similar fashion, at which point overpatch 2301 is fully adhered to the skin of the wearer.

FIG. 43J-1 is an exploded view showing another example embodiment of overpatch assembly 2300-3 with overpatch 2301 and liners 2314-2 and 2315-2 in their unassembled and unfolded states. Here, pull tabs 2316 and 2317 are wider with more surface area for the user to grasp when removing liners 2314-2 and 2315-2, as compared to the embodiment of FIGS. 431-1 and 43I-2. Also, in this embodiment open regions 2348 and 2349 each are relatively larger than in the embodiments of FIGS. 43I-1 and 431-2, with the open regions 2348 and 2349 being larger than the corresponding interior space (or open region) 2313 of layer 2303 of the overpatch assembly 2300-3 in at least one dimension, which in this example is parallel with the X axis. This dimension is parallel to the direction liners 2314-2 and 2315-2 are pulled for removal.

The terminus of edge 2345 of liner 2315-2 is the last location of edge 2345 to be removed from overpatch 2301. As shown in FIG. 43J-2, where liner 2315-2 is in a state of being removed while liner 2314-2 is still adhered to layer overpatch 2301, the terminus of edge 2345 is adhered to a location on overpatch 2301 that is offset laterally (e.g., in length and/or width as opposed to height) in width from the edge 2347 of interior space 2313 at the ultimate point of release of edge 2345. Edge 2341 of liner 2314-2 is shown in dashed line as it is obscured by second portion 2335-2 of liner 2314-2. Edge 2341 is also laterally offset from the edge 2347 of interior space 2313. The region of adhesive exposed through first portion 2335-1 of liner 2314-2 and in contact with second portion 2335-2 is labelled as region 2354. A similar region will be present through portion 2336-1 of liner 2315-2. When assembly 2300-3 is assembled, the edge of space 2313 is not aligned with edges 2341 and 2345 but rather edges 2341 and 2345 are adhered to overpatch 2301 at a location surrounded by adhesive on layer 2303. Offsetting the termini of edges 2341 and 2345 from edge 2347 of interior space 2313 facilitates removal of liners 2314 and 2315, respectively, from overpatch 2301. Removal of liners with edges that are not offset is more likely to result in folding, pleating, or tearing of the liners and/or overpatch 2301 in the vicinity of the ultimate location of detachment.

Edges 2341 and 2345 can be offset in a number of different ways using a number of different configurations of liners 2314 and 2315 with varying shapes of open regions 2348 and 2349. Open regions 2348 and 2349 can be larger than open region 2313 in one or more directions but not all directions, e.g., as shown in the embodiments of FIGS. 43J-1 through 43J-6. In the embodiment of FIGS. 43J-1 and 43J-2, open regions 2348 and 2349 are generally semi-circular near portions 2335-2 and 2336-2, respectively, and have a rounded polygonal shape near portions 2335-1 and 2336-1, where that polygonal shape tapers to the position of the terminus of edges 2341 and 2345 that is last to be removed from overpatch 2301. The embodiment of overpatch assembly 2300-4 of FIGS. 43J-3 and 43J-4 is similar but the terminus of edge 2341 of liner 2314-3 and the terminus of edge 2345 of liner 2315-3 are each pointed as opposed to rounded. Edges 2341 and 2345 of overpatch assembly 2300-3 of FIGS. 43J-1 and 43J-2 can be referred to as V-shaped with a rounded terminus. Edges 2341 and 2345 of overpatch assembly 2300-4 of FIGS. 43J-3 and 43J-4 can be referred to as V-shaped with a pointed terminus. Other polygonal and/or rounded shapes can be used as well, with or without rounded corners. In the embodiments of FIGS. 43J-1 through 43-J4, the perimeter edges 2341 and 2345 are asymmetrical with respect to creases or folds 2337 and 2338, respectively. The additional adhesive exposed though portions 2335-1 and 2336-1 in region 2354 is not accessible to the user when in the folded state, as shown by folded liner 2314-2 of FIG. 43J-2. The additional exposed adhesive of region 2354 acts to adhere portion 2335-2 to portion 2335-1 when liner 2314-2 (or 2314-3) is in the folded state, and a similar situation applies for liner 2315-2. This results in a lower profile, more compact, flatter state as shown in the side view of FIG. 43J-4, where portions 2335-2 and 2336-2 are held adjacent to and in contact with portions 2335-1 and 2336-1, respectively. This lower profile state can improve the user experience as the folded liners are kept flat and are not free to deflect as shown in FIG. 43H. Additional regions can be exposed in portion 2335-1 to allow securement to portion 2335-2, such as regions at or adjacent to the periphery of overpatch 2301. In one example embodiment, a region of portion 2335-1 of liner 2314 at the periphery is omitted at a location where overpatch 2301 can adhere to portion 2335-2 adjacent pull tab 2316, and similarly for liner 2315. This permits even better adhesive contact between portions 2335-2 and 2336-2 and adhesive layer 2303 of overpatch 2301.

FIGS. 43J-5 and 43J-6 show another example embodiment of overpatch assembly 2300-5 having liners 2314-4 and 2315-4 with open regions 2348 and 2349 that are symmetrical with respect to creases or folds 2337 and 2338, respectively. Liner 2314-4 has an elliptical or oval open region 2348 with edges that will fully align when folded over. As a result, region 2350 of adhesive is exposed through both portions 2335-1 and 2335-2 of liner 2314-4, and a similar region of adhesive will be exposed through both portions 2336-1 and 2336-2 of liner 2315-4. FIGS. 43J-7 and 43J-8 show another example embodiment of overpatch assembly 2300-6 having liners 2314-5 and 2315-5 with open regions 2348 and 2349 that are symmetrical with respect to creases or folds 2337 and 2338, respectively. Liner 2314-5 has a circular open region 2348 that is symmetrical in all directions with edges that will fully align when folded over. As a result, region 2350 of adhesive is exposed through both portions 2335-1 and 2335-2 of liner 2314-5, and a similar region of adhesive will be exposed through both portions 2336-1 and 2336-2 of liner 2315-5. Here, the circular open regions 2348 and 2349 of assembly 2300-6 have the same shape as interior space 2313 but with a larger width or diameter. In other embodiments, the open regions 2348 and 2349 can have a different shape from interior space 2313 but again be larger in all directions.

These symmetrical open regions of assemblies 2300-5 and 2300-6 expose additional adhesive on overpatch 2301 in regions 2350 that, like regions 2318, increase the tendency of assembly 2300 to adhere to the skin when first applied over the housing 103 but prior to removal of liners 2314 and 2315. This can facilitate the user experience and ease of placement. Liner 2315 can be configured with the same shape of open region 2349 as that of open region 2348 of liner 2314, or liner 2315 can be configured with a different shape for open region 2349 from that of open region 2348 of liner 2314.

FIG. 43K-1 shows a bottom up view of an example embodiment of a package 2328-1 in which overpatch assembly 2300-2 can be shipped and provided to the user. Here package 2328-1 is configured as a sleeve. Package 2328-1 has a liner surface 2329 (e.g., such as a strip) located within that is in a position corresponding to gap 2319 and regions 2318 where adhesive is exposed. Overpatch assembly 2300-2 is adhered to liner surface 2329 while within package 2328-1 which stabilizes its location during shipping. Removal of overpatch assembly 2300-2 removes exposed adhesive layer in gap 2319 and regions 2318 from liner surface 2329 of package 2328-1 and the assembly 2300-2 is ready for application to the skin by the user. In another embodiment, assembly 2300-2 can be provided with a removable liner (not shown) over gap 2319 and regions 2318 and the user can remove the assembly 2300-2 from its packaging and then remove the liner to expose the adhesive. In other embodiments, if additional regions of adhesive are exposed (e.g., region 2350) and/or if a different sized region of adhesive is exposed, the size of liner surface 2329 can be changed accordingly to encompass all of the exposed adhesive. Additionally or alternatively, multiple instances of liner surfaces 2329 can be present (on one or both sides of package 2328 as appropriate) to cover all regions of exposed adhesive.

FIG. 43K-2 shows a bottom up view of another example embodiment of a package 2328-2 in which overpatch assembly 2300-2 can be shipped and provided to the user. Here package 2328-2 is similar to package 2328-1 but package 2328-2 further includes an element 2320 that is electrically conductive. When package 2328-2 is included within a larger product or product package, such as a that for a kit including overpatch assembly 2300 and an on-body device and/or an inserter (or applicator), a sensor can detect the presence of element 2320 and thereby verify the presence of package 2328-2 within the larger package. Such a sensor could, for example, be present at the manufacturer and/or at the distributor and can act as part of a quality check process to ensure no larger kit package leaves the possession of the manufacturer and/or distributor without an overpatch assembly 2300 included therein. Element 2320 is electrically conductive so as to, for example, permit an electrical current to flow therein when exposed to an electromagnetic field applied by an inductor or other device. The induced current can then be sensed by the sensor and used to verify presence of package 2328-2.

In this embodiment element 2320 is configured as a metallic foil tape that is adhered to the side of package 2328-2 opposite the side having liner surface 2329. In another embodiment, element 2320 can be placed adjacent liner surface 2329 within the package wall such that any sharp edges of element 2320 are contained within the wall and not exposed to a user. Element 2320 can be placed along a side (as shown here) and/or along an edge of package 2328-2, either in the interior or exterior of package 232802. Element 2320 can alternatively be a component of overpatch assembly 2300 such that sensing for presence applies to the overpatch assembly itself and not the package containing the overpatch assembly. Element 2320 can be, for example, coupled with at least one of liners 2314 and/or 2315.

In this embodiment, element 2320 is or includes aluminum, but is or includes a different material in other embodiments, such as copper, steel, carbon or a carbon containing compound, and the like. In this embodiment element 2320 is configured as a strip or layer of highly flexible material, but can be configured differently in other embodiments, such as a wire, thread, mesh, and the like. Element 2320 can be adhered directly to the package or contents therein, or can be embedded within the package or contents therein, or coupled using fasteners or other means. Element 2320 can be applied in bulk to the material of package 2328-2 (e.g., paper, plastic, or the like) such that element 2320 is already present and attached when the material of package 2328-2 is cut to shape for package fabrication. In one embodiment, the material of package 2328-2 itself is electrically conductive and doubles as both package and element 2320.

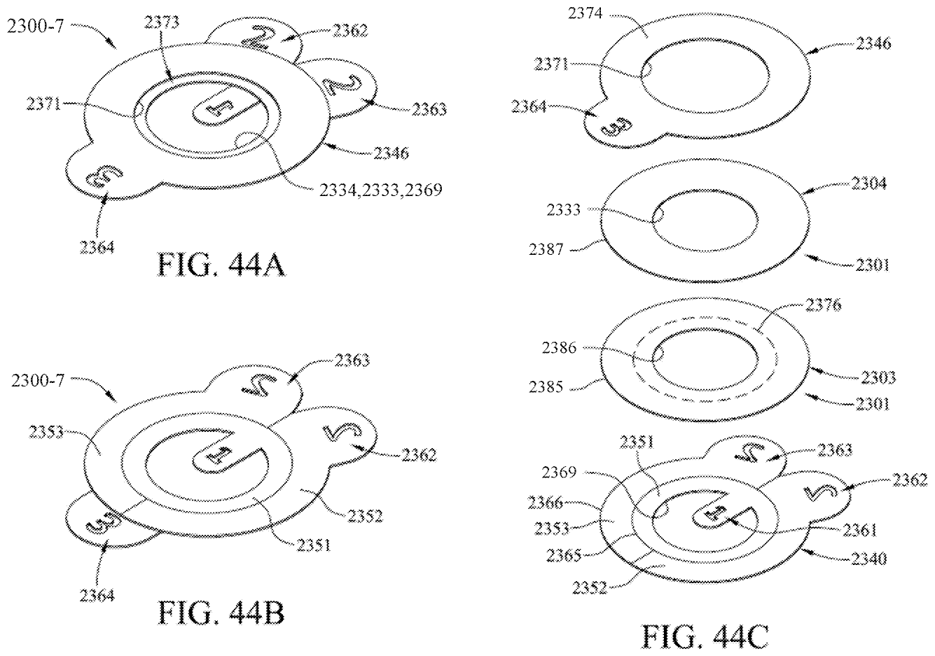

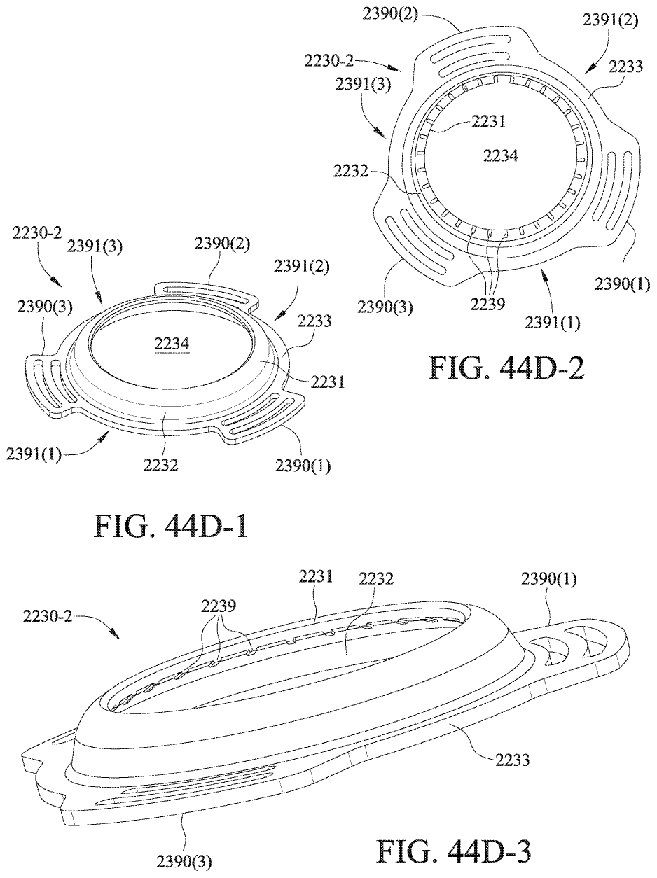

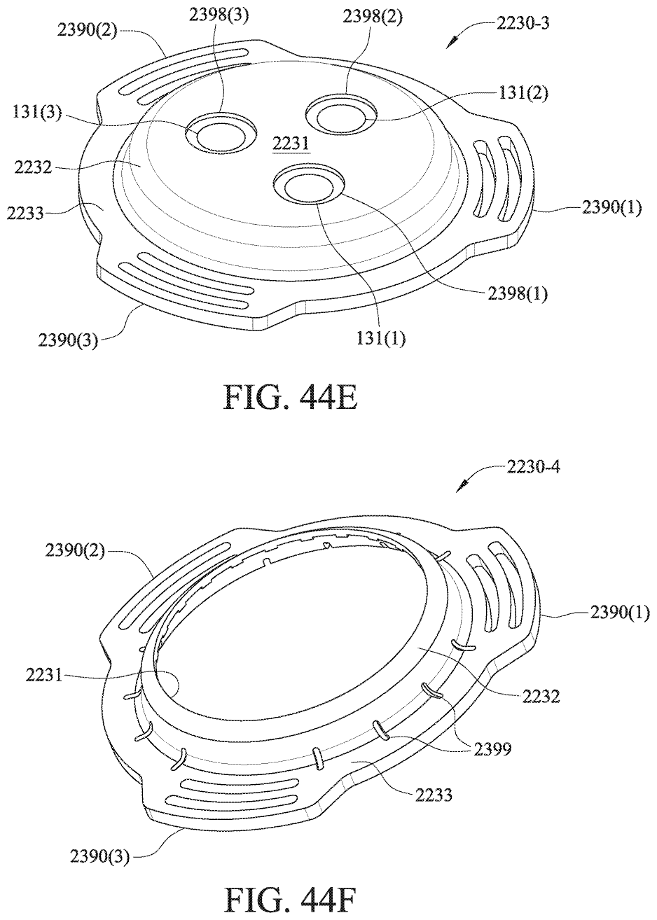

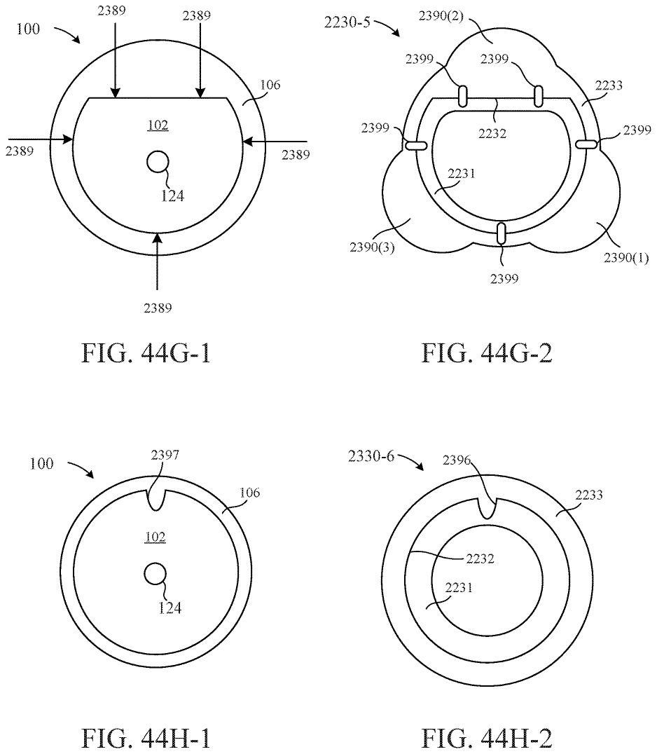

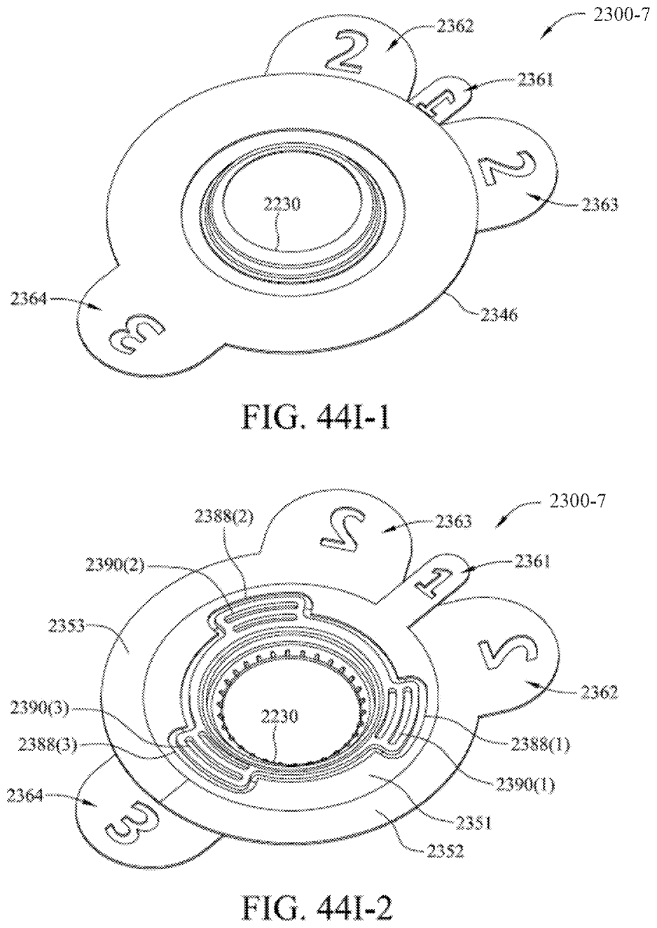

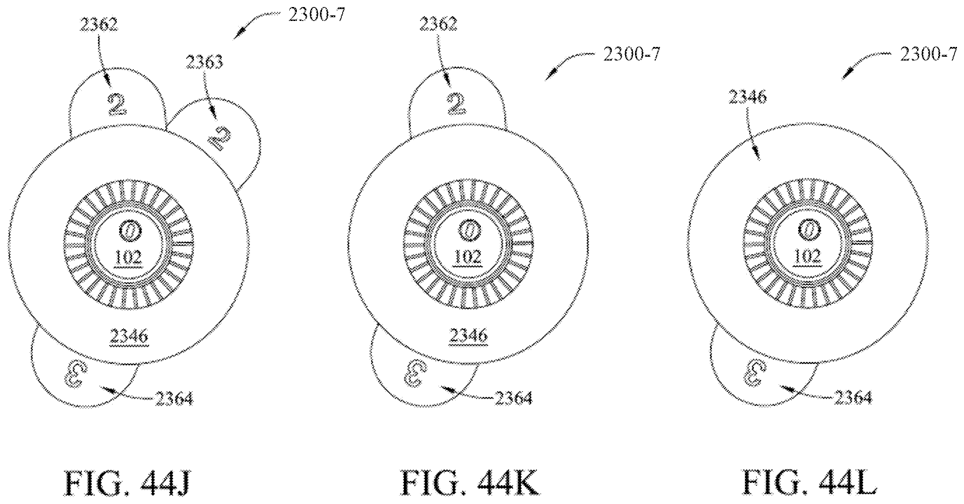

FIG. 44A shows a top down perspective view of another overpatch assembly 2300-7 according to an example embodiment. FIG. 44B shows a bottom up perspective view of overpatch assembly 2300-7 and FIG. 44C shows an exploded view of the components of overpatch assembly 2300-7. FIG. 44D-1 shows a perspective view of support structure 2230-2 according to an example embodiment. In this embodiment support structure 2230-2 is provided to a user with overpatch assembly 2300-7 as separate unassembled components in a package. The user will assemble the two components into an assembled version of the overpatch assembly 2300-7 prior to application to the user's or another on-body device wearer's body.

With reference to FIG. 44C, in this embodiment assembly 2300-7 includes a lower liner assembly 2340, a skin adhesive layer 2303, a backing layer 2304, and a removable stiffener layer 2346. Lower liner assembly 2340 includes a first liner 2351, and one or more secondary liners. In this embodiment, lower liner assembly includes a first secondary liner 2352, and a second secondary liner 2353. Lower liner assembly has an outer peripheral edge 2366. Each of liners 2351-2353 are generally coplanar in this embodiment although more complex overlapping constructions can also be used. First liner 2351 is located within an interior space of lower liner assembly 2340 that is formed by liners 2352 and 2353 arranged on opposite sides of first liner 2351. First liner 2351 has an annular configuration with a pull tab portion 2361 extending into and open center space of lower liner assembly 2340. Liner 2352 has a pull tab portion 2362 extending in an opposite direction away from the center space of lower liner assembly 2340. Similarly, liner 2353 includes a pull tab portion 2363 that also extends in the opposite direction away from the center space of lower liner assembly 2340. In this embodiment, liner 2352 and liner 2353 can be combined into a single secondary liner that extends about the periphery of first liner 2351 and is removable in a manner similar to that described with respect to liner 2302 of FIG. 43E.

The overpatch 2301 is formed by at least two layers which in this embodiment are backing layer 2304 and underlying adhesive layer 2303. The two layers do not have to be structurally distinct. In this embodiment and all other embodiments described herein, an adhesive layer (e.g., layer 2303) can be a coating or applied chemical that adheres to the lower side of another layer (e.g., backing layer 2304) but that does not itself has a separate and distinct structural form (e.g., annular) as depicted here. Adhesive layer 2303 has an outer peripheral edge 2385 and an inner peripheral edge 2386. Backing layer 2304 has an outer peripheral edge 2387 and an inner peripheral edge 2333.

Stiffener layer 2346 is configured to be placed on the upper side of backing layer 2304. Stiffener layer 2346 can include a material that is relatively more rigid than the material combination of layers 2304, 2303 and 2340 and thus provides a more rigid structure that the user can orient and handle without prematurely adhering to skin or another object. Stiffener layer 2346 can include an adhesive layer (not shown, located on the side opposite to side 2374) to assist in adhering to backing layer 2304 and a pull tab portion 2364 that extends away from the central space of assembly 2300-7. Stiffener layer 2346 can, in some embodiments, be configured in a manner similar to unit 2309 described with respect to FIG. 43B, and can optionally include a liner with pull tab portion adhered to a stiffener layer, which in turn can adhere to backing layer 2304. Each of pull tab portions 2361-2364 can include an indicium indicating order of removal similar to the embodiments described with respect to FIGS. 43D and 43E.

Referring to FIG. 44D-1, support structure 2230-2 includes upper retainer structure 2231, sidewall 2232, and lower retainer structure 2233, which together form an interior space 2234 in which an on-body device housing (not shown) can be received. Here, support structure 2230-2 has an annular or ring shape, but can have other shapes in other embodiments such as a dome shape or bar shape. Upper retainer structure 2231 is configured as a rim or lip that, along with sidewall 2232, can contact and overlie at least a portion of the top and side surfaces of the on-body device housing to assist the user in locating and aligning overpatch 2301 over the on-body device housing and then to subsequently assist in support of the on-body device housing while on the body.

Lower retainer structure 2233 includes one, two, three, four, five, six, or more extension sections 2390 that can extend radially away from interior space 2234 and can provide surface area for overpatch adhesive layer 2303 to adhere to support structure 2230-2. Support structure 2230-2 is coupled with overpatch 2301 by adherence of skin adhesive layer 2303 to the upper surface of extensions sections 2390 and also by adherence of the remainder of skin adhesive layer 2303 to the skin, which holds support structure 2230-2 pressed against the skin. The one or more extension sections 2390 can be adjacent to one or more gap regions 2391 where no material of support structure 2230-2 is present. Gap regions 2391 can permit adhesive layer 2303 to be accessed from the skin facing side of overpatch 2301 as will be described in more detail. In this embodiment, support structure 2230-2 includes three extension sections 2390 with three gap regions 2391, where one gap region 2390(1) is located between extension sections 2390(1) and 2390(3), a second gap region 2390(2) is located between extension sections 2390(1) and 2390(2), and a third gap region 2390(3) is located between extension sections 2390(2) and 2390(3). Extension sections 2390 give support structure 2230-2 a petal-shaped configuration.

Upper retention structure 2231 does not overlap the entire top surface of an on-body device housing and thus can allow moisture to vent more easily from the top and/or surrounding region of the on-body device. The on-body device may include vent passageways 111 with vent outlets 131 that vent from the upper portion of the on-body device, and thus the open upper retention structure 2231 can facilitate this venting. If the upper retention structure 2231 extends across the top surface of the on-body device housing, such as in a dome shape, then upper retention structure 2231 can include one or more apertures in either closed (e.g., circular or oval) or open (e.g., as an open-ended notch or slot) aligned with the vent outlets.

FIG. 44D-2 shows a bottom up view of support structure 2230-2 showing the undersurface of upper retention structure 2231 having multiple standoffs 2239. FIG. 44D-3 shows a perspective view of support structure 2230-2 again showing the upper retention structure 2231 with multiple standoffs 2239. Standoffs 2239 are downwardly extending projections that are configured to contact the top surface of the on-body device housing (not shown) and create a gap in the spaces between adjacent standoffs through which moisture can vent through space 2234 from the volume around the on-body device and out through the open top of support structure 2230-2.

FIG. 44E shows a top down perspective view of an example embodiment of support structure 2230-3 having a dome shape with three apertures 2398 aligned with vent outlets 131 of an on-body device therein. Any number of apertures 2398 can be present and can be optionally equivalent in size and aligned with each of the vent outlets on the top side of the on-body device housing.

Additionally or alternatively, support structure 2230 can include one or more side apertures in either closed (e.g., circular or oval) or open (e.g., as an open-ended notch or slot) configurations to allow venting of moisture from the side and/or underside of the on body housing. Mechanisms for venting moisture from the side and/or underside of the on-body device are described herein and include moisture-transporting channels 850, 855 and side-exiting vent outlets 131 (see, e.g., FIGS. 2I-1, 2I-2, 2I-4, 2I-5, 3H-1, and 3H-2). FIG. 44F shows a top down perspective view of an embodiment of support structure 2230-4 that includes apertures 2399 distributed at regular intervals around the periphery of sidewall 2232. These apertures 2399 can align with the peripheral terminations of channels 850, 855 or side-exiting vent outlets 131 for transporting moisture as described herein. For example, each peripheral termination of channels 850, 855 or each location of side-exiting vent outlet 131 can be aligned with an aperture 2399 in support structure 2230-3 such that there are the same number of apertures 2399 as peripheral channel terminations and/or side-exiting vent outlets.

Support structure 2230 can be configured to mate with on-body device housing in a manner that ensures alignment between venting features on support structure 2230 and corresponding venting features on the on-body device. The self-alignment of support structure 2230 and on-body device 100 can be accomplished according to a number of different embodiments. In some embodiments, the implementation of the on-body device housing can have a peripheral profile shape without rotational symmetry, for example such as the embodiments of shapes depicted in FIGS. 1N-3, 1N-4, 1N-5, 1N-10, 1N-11, 1N-13 (or others), and the interior of support structure 2230 can have a corresponding shape for the interior recess such that placement of support structure 2230 over the on-body device 100 results in self-alignment. FIG. 44G-2 shows a bottom up view of an embodiment of support structure 2230-5 having an interior recess formed by sidewall 2232 with a shape complementary to an on-body device housing shape such as that depicted in FIG. 44G-1 (see also FIG. 1N-4). The location of apertures 2399 in sidewall 2232 are indicated by arrows, and those locations will align with the corresponding locations of channel terminations or vent outlets in on-body device 100 that are indicated by arrows 2389.

Also, or alternatively, support structure 2230 and on-body device 100 can each include a physical or structural alignment feature that interfaces with a corresponding alignment feature on the other, such as a male and female configuration. FIG. 44H-2 shows a bottom up view of support structure 2230-6 having a projection or detent 2396 that is configured to be received in a corresponding recess 2397 in on-body device 100 of FIG. 44H-1. Other configurations of alignment features can be used, and can the alignment feature on the on-body device can take advantage of pre-existing features. For example, if sharp aperture 124 in the top surface 108 of an on-body device 100 (as described elsewhere herein) is in a location that provides alignment (such as offset from the center of an on body device having rotational symmetry), then sharp aperture 124 can be utilized as recess 2397 and can interface with a corresponding projection 2396 on support structure 2230.

Referring back to FIG. 44A, when assembly 2300-7 is assembled the inner peripheral edge 2334 of adhesive layer 2303 and the inner peripheral edge 2333 of backing layer 2304 can be in alignment, or in close proximity (e.g., within 1-5 millimeters) of each other. An inner peripheral edge 2369 of liner 2351 can also be aligned with edges 2334 and 2333 such that all three terminate at the same or generally same location. An outer peripheral edge 2365 of liner 2351 and an inner peripheral edge 2371 of stiffener layer (or unit) 2346 can terminate at the same location, or in close proximity (within 1-5 millimeters) of each other. In this configuration, a portion 2373 of backing layer 2304 is exposed on the upper side of assembly 2300-7. When liner 2351 is removed, a first portion 2376 of adhesive layer 2303 will be exposed beneath liner 2351.

In an embodiment where assembly 2300-7 is supplied to a user partially unassembled, then the user can adhere support structure 2230-2 to the exposed skin adhesive of the first portion 2376 of layer 2303, such as by pressing the top surface of extensions sections 2390 against the first portion 2376 of the skin adhesive. The inner peripheral edges 2334 and 2333 of layers 2303 and 2304 can by slightly larger than the sidewall circumference and can serve to align with sidewall 2232 of support structure 2230-2 such that when the user inserts the support structure 2230-2 into the open interior region of assembly 2340-2 the two entities self-align.

In an embodiment where assembly 2300-7 is supplied to a user with support structure 2230-2 already adhered to assembly 2300-7, such as that depicted in FIGS. 441-1 and 441-2, then the user need not assemble the two pieces and liner 2351 can include cutaways 2388 on the interior edge to accommodate extension sections 2390. Here, pull tab portion 2361 extends radially outwardly from the center region of assembly 2300-7 to accommodate the presence of the pre-adhered support structure 2230. In another embodiment, liner 2351 is omitted altogether and the overpatch assembly 2300-7 can be packaged with the first portion 2376 of the adhesive layer in contact with a liner portion of the package such that removal of assembly 2300-7 from the package exposes the first portion of the adhesive. In this embodiment assembly 2300-7 can be pre-assembled or the user can adhere support structure 2230-2 to assembly 2300-7 after removal from the package.

In some embodiments of overpatch assembly 2300-7, secondary liners 2352 and 2353 can be configured to be relatively rigid, such as by use of a stiffer material or by folding the liners, and thus can allow stiffener layer 2346 (or stiffener unit) to be omitted. Folded liners of this type are described in more detail with reference to FIGS. 43F-43J-8.

Methods of applying overpatch assembly 2300-7 are described with respect to FIGS. 44J thru 44L. The support structure 2230 of the overpatch assembly 2300-7 can be positioned by a user on a housing of the on-body device while the first portion 2376 of adhesive layer 2303 of the overpatch assembly 2300-7 (or overpatch) is exposed and a second portion of the adhesive layer is unexposed (the portion underneath the secondary liners 2352 and 2353). Alternatively, the user can remove a first skin adhesive liner 2351 to expose the first portion of the adhesive layer prior to positioning the support structure of the overpatch assembly on the housing of the on-body device.

In some embodiments, positioning can include positioning the support structure in contact with the housing of the on-body device. In some embodiments, positioning can include aligning the support structure over the housing of the on-body device, for example, by aligning the peripheral shape of the housing of the on-body device with a corresponding peripheral shape of a recess within the support structure (see, e.g., FIG. 44G), and/or can include aligning a physical alignment feature of the housing of the on-body device with a corresponding inverse physical alignment feature of the support structure (see, e.g., FIG. 44H). Positioning the support structure of the overpatch assembly on the housing of the on-body device can include sliding the support structure of the overpatch over the housing of the on-body device such that the housing of the on-body device is received within an interior space of the support structure.

The first portion of the adhesive layer can then be adhered to skin of the wearer while the support structure is on the housing and the second portion of the adhesive layer is unexposed. Adhering can include manually pressing a side of the overpatch assembly opposite to the first portion of the adhesive layer, such as portion 2373 of backing layer 2304, to adhere the first portion of the adhesive layer to the skin. FIG. 44J shows a top down view of assembly 2300-7 being positioned and adhered to the skin. A user can press on the regions of backing layer 2304 over gap regions 2391 where adhesive layer 2303 is exposed and capable of adhering to the skin. The first portion that is adhered to the skin in this embodiment is that portion of adhesive layer 2303 exposed in the gap regions 2391 between extension sections 2390. The first portion of the adhesive layer can thus be non-contiguous in some embodiments. If the user adheres the first portion to the skin in a manner such that assembly 2300-7 is in a less than ideal position, or mis-positioned, then the user can reposition assembly 2300-7. In some embodiments, this can include un-adhering the first portion from the skin, moving assembly 2300-7 into a better position, and then re-adhering the first portion to the skin. Repositioning of the assembly 2300-7 is facilitated by only partially exposing adhesive layer 2303 with the removal of first liner 2351.

Once assembly 2300-7 is positioned in the desired manner and location, the user can remove any secondary liners. In this embodiment, the user can remove first secondary liner 2352 (see FIG. 44K) (e.g., by pulling pull tab 2362) which unfolds and slides liner 2352 out from the space between adhesive layer 2303 and the skin. The portion of adhesive layer 2303 that was contacting liner 2352 is exposed and can then be adhered to the skin, such as by manually pressing the opposite side of assembly 2300-7. Liner 2353 can then be removed in similar manner and the remainder of adhesive layer 2303 can be adhered to the skin by pressing. At this time, assembly 2300-7 has been fully adhered to the skin (see FIG. 44L). If a stiffener layer 2346 (or unit) is present, then the user can remove this stiffener such as by pulling pull tab 2364.

In these embodiments, support structure 2230 and all variations thereof can be configured as a reinforcement device as described herein and/or with respect to FIGS. 31-40B.

The embodiments of patches described in this section and/or with respect to any and all of FIGS. 43A-44L can be used in combination with any and all of the on-body devices, reinforcement devices, patches, analyte monitoring systems, and/or methods described in the preceding sections and/or with respect to FIGS. 1-42P and FIGS. 45A-46G.

### Multi-Layer Patch

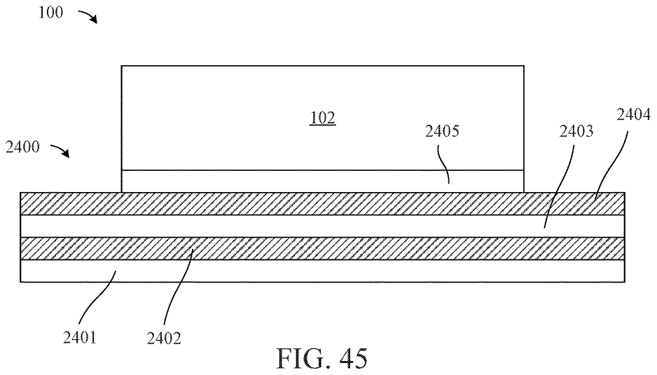

In all embodiments herein, the adhesive patch (or adhesive patch assembly 1050) in the first configuration as an on-body device patch (e.g., patch 106), can have five or more layers. As described herein, at least one layer can be a non-isotropic layer, but such is not required. FIG. 45 shows a side view of a construction of an on-body device 100 with an adhesive patch 2400 according to an example embodiment having five layers: a first adhesive layer 2401, a first structural layer 2402, a second adhesive layer 2403, a second structural layer 2404, and a third adhesive layer 2405. In other embodiments more than five layers can be present. This embodiment of patch 2400 can alternatively be used as the overpatch (or overpatch assembly) with omission of adhesive layer 2405 (thereby having four layers instead of five). Whether configured as a main patch or overpatch, first structural layer 2402, second adhesive layer 2403, and second structural layer 2404 can be considered to be or be part of a backing. First adhesive layer 2401 functions primarily as a skin adhesive layer, second adhesive layer 2404 functions primarily to adhere first structural layer 2042 to second structural layer 2404, and third adhesive layer 2405 functions to adhere the on-body device housing 102 to patch 2400. This construction of patch 4500 can exhibit sufficient wear duration to last an extended period of time, such as the wear durations described herein, on the skin of the wearer. Patch 4500 can exhibit an MVTR of 115 grams/square meter/day or higher. In some embodiments patch 4500 exhibits an MVTR of 175 grams/square meter/day or higher and in some embodiments patch 4500 exhibits an MVTR of 200 grams/square meter/day or higher.

In embodiments, first structural layer 2402 and second structural layer 2404 can each be a polymeric or plastic material such as, but not limited to polyesters such as polyethylene terephthalate (PET), polyurethanes (PU), polyethylenes (PE), polyethers, elastanes, polyamides such as nylon, and combinations thereof. In some embodiments, one or both of first structural layer 2402 and second structural layer 2404 is or includes a polyurethane. In some embodiments, one or both of first structural layer 2402 and second structural layer 2404 is or includes a polyester such as PET. In some embodiments, one or both of first structural layer 2402 and second structural layer 2404 is or includes a polyurethane, while the other is or includes a polyester such as PET. In some embodiments, first structural layer 2402 is or includes a polyurethane and second structural layer 2404 is or includes a polyester such as PET. In some embodiments, first structural layer 2402 is or includes a polyester such as PET and second structural layer 2404 is or includes a polyurethane. In each of the aforementioned material embodiments, first structural layer 2402 can be a non-isotropic layer and second structural layer 2404 can be an isotropic layer, first structural layer 2402 can be an isotropic layer and second structural layer 2404 can be a non-isotropic layer, both first structural layer 2402 and second structural layer 2404 can be an isotropic layer, or both can be a non-isotropic layer.

The adhesive layers 2401, 2403, and 2405 may be or include an acrylic adhesive such as a medical extended wear acrylic adhesive (e.g., manufactured by 3M). The adhesive layers may include other adhesives such as silicone adhesives, polyisobutylene and other hydrocarbon adhesives, polyurethane adhesives, natural rubber adhesives, synthetic rubber adhesives, and the like.

In some embodiments, first adhesive layer 2401 and first structural layer 2402 can be fabricated as a first length of tape, such as a medical grade skin tape and second adhesive layer 2403 and second structural layer 2404 can also be fabricated as a second length of tape. The two tapes can then be adhered to each other to provide the layered configuration shown in FIG. 45. In one embodiment, the first length of tape with layers 2401 and 2402 is a first material combination, and the second length of tape with layers 2403 and 2404 is a second material combination different from the first. In another embodiment, the first length of tape with layers 2401 and 2402 is a first material combination, and the second length of tape with layer 2403 and layer 2404 is a second material combination different from the first. The second material combination, in one example embodiment, may be a low tack adhesive for layer 2403 such as AVERY DENNISON FT-310 and layer 2404 may be a polymer such as MELINEX PET. A release liner for adhesive 2401 can be a paper-based silicon coated release liner or otherwise. Patches 2400 can then be trimmed from the joined tape to form the final product. The third adhesive layer 2405 can be added before or after trimming, or can be a layer of the second tape that is joined to the first tape. Third adhesive layer 2405 can be the same size as patch 2400 or can be smaller as shown here, depending on the configuration of the on-body device 100.

Each of layers 2401-2405 can have a thickness selected for targeted benefits of durability, longevity, and moisture permeability. In an example embodiment, first adhesive layer 2401 has a thickness of 4 mils+/−25%, first structural layer 2402 has a thickness of 11 mils+/−25%, second adhesive layer 2403 has a thickness of one mil+/−25%, and second structural layer 2404 has a thickness of 0.8 mils+/−25%. In another example embodiment, first adhesive layer 2401 has a thickness of 4 mils +/−10%, first structural layer 2402 has a thickness of 11 mils+/−10%, second adhesive layer 2403 has a thickness of one mil+/−10%, and second structural layer 2404 has a thickness of 0.8 mils+/−10%. The thicknesses can be nominal. The dimensions set forth here of for example embodiments and do not limit the scope of the present disclosure.

The embodiments of main body patches described in this section and/or with respect to FIG. 45 can be used in combination with any and all of the on-body devices, reinforcement devices, other main body patches, analyte monitoring systems, and/or methods described in the preceding sections and/or with respect to the embodiments shown in FIGS. 1-44L and 46A-46G.

### On-Body Devices, Systems, Methods, and Applications

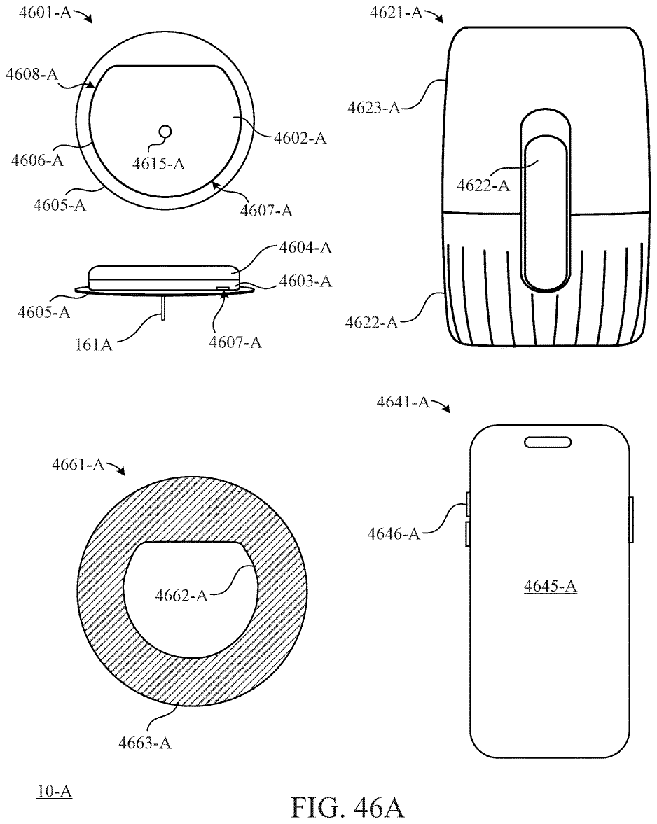

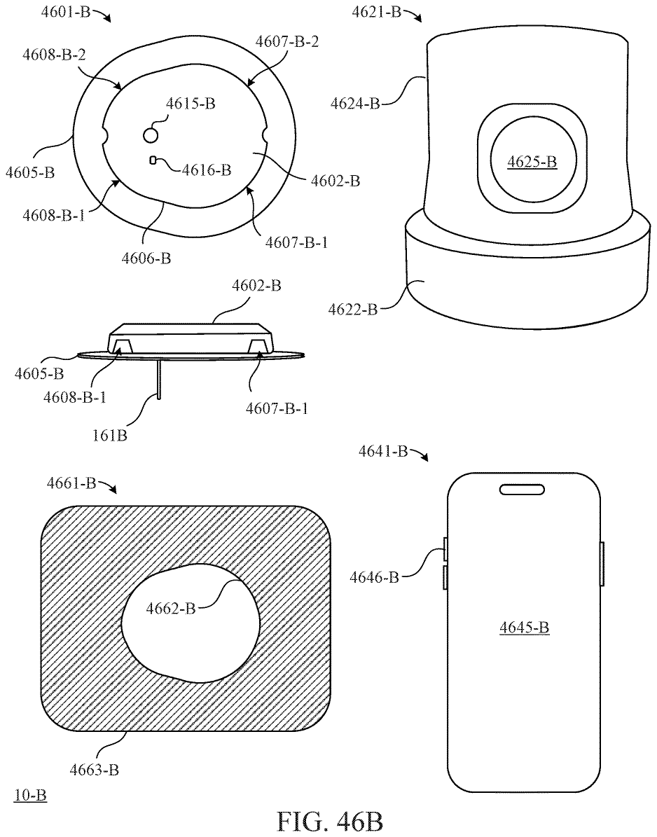

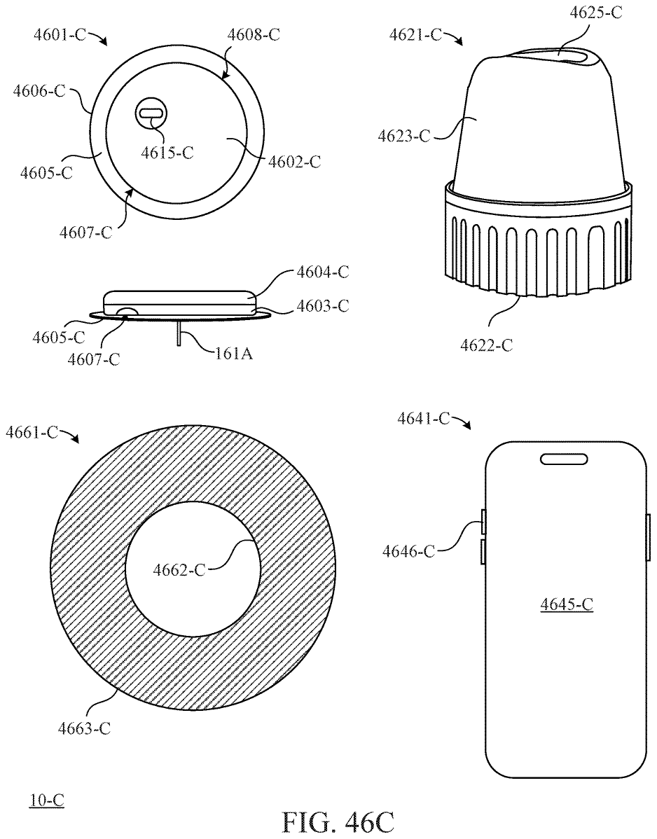

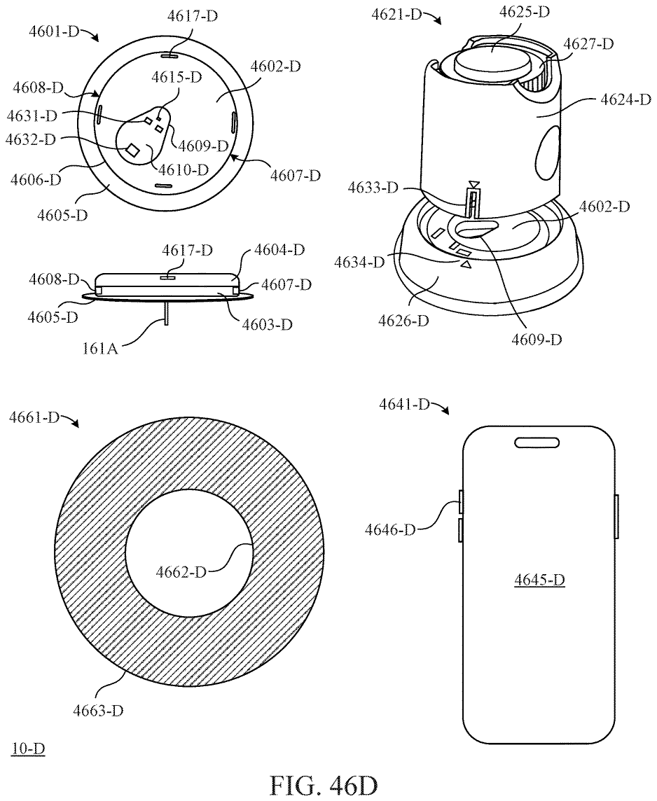

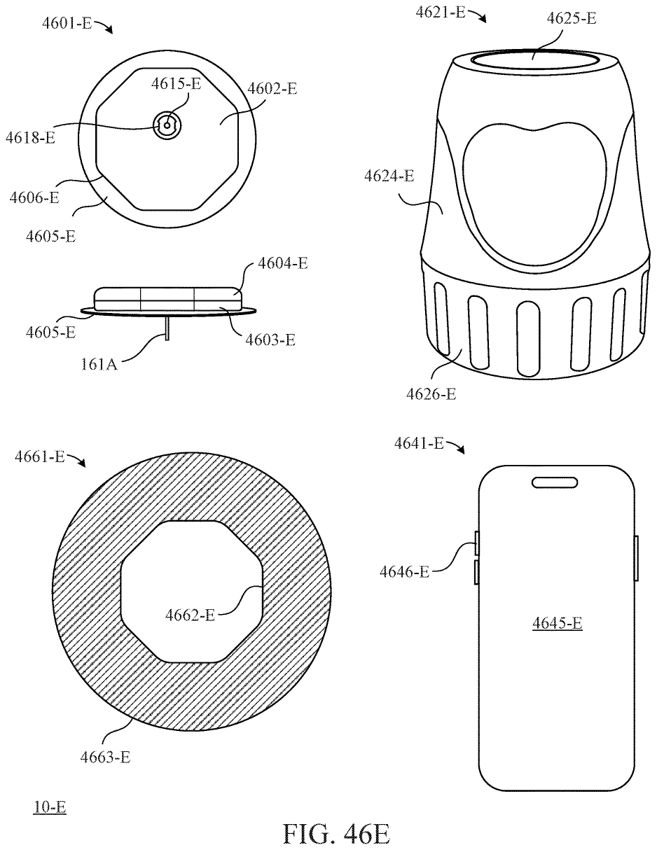

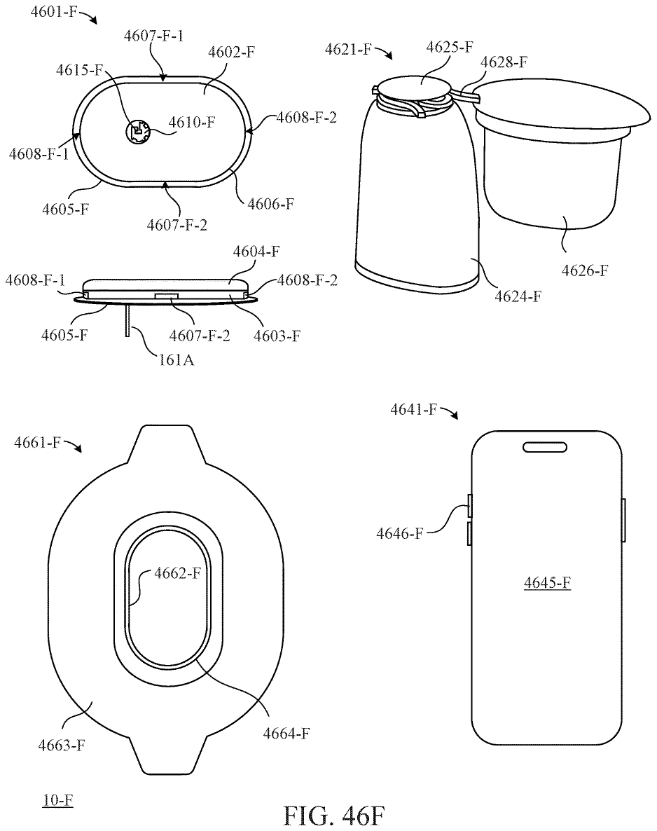

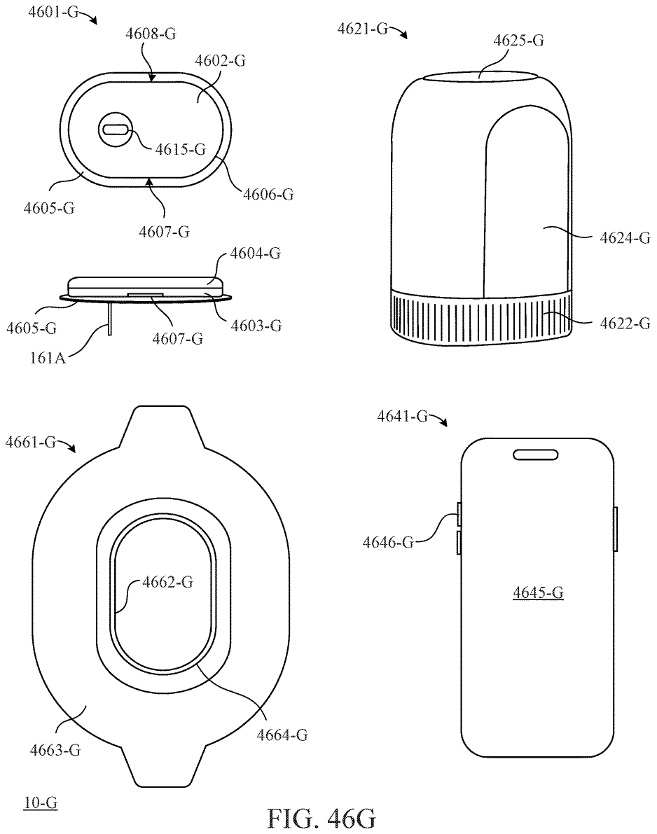

As stated, the subject described herein can be combined in a wide variety of ways to arrive at various implementations of on-body devices, systems, methods of use, and applications. Analyte monitoring system 10 as described herein with reference to FIG. 1A and elsewhere can include an on-body device implemented in accordance with any of the embodiments herein, a receiver unit implemented in accordance with any of the embodiments herein, and/or a secondary receiver unit implemented in accordance with any of the embodiments herein, in communication with server 50 over network 40. Analyte monitoring system 10 may also include an inserter implemented in accordance with any of the embodiments herein and/or an applicator implemented in accordance with any of the embodiments herein. System 10 may include one or more sensors configured to be partially implantable, wholly implantable, or wholly ex vivo. A set of non-exhaustive example embodiments of systems 10 are described with reference to FIGS. 46A-H. These non-exhaustive example embodiments are illustrative of just a few of the many combinations encompassed by the subject matter described herein.

### System 10-A

An example embodiment of such a system 10-A is shown in the landscape view of FIG. 46A. Here, system 10-A includes on-body device 4601-A, inserter 4621-A, receiver unit 4641-A, and/or overpatch 4661-A. On-body device 4601-A can be configured in accordance with any of the embodiments of on-body device 100 described herein. On-body device 4601-A is shown here in both top down and side views. Here, on-body device 4601-A includes a housing 4602-A having a first part 4603-A and a second part 4604-A attached together. In this embodiment, the peripheral boundary 4606-A of housing 4602-A is configured like the embodiment described with respect to FIG. 1N-4, with a circular boundary side and a straight boundary side. The shape of each side may be interrupted by features. For example, housing 4602-A may include two or more recesses 4607-A and 4608-A adapted to interface with projections of inserter 4621-A, e.g., such as on deflectable arms, that are configured to releasably hold on-body device 4601-A within inserter 4621-A, for example, as described with respect to the embodiments of FIGS. 1U-16 through 1U-22. Housing 4602-A includes a sharp aperture 4615-A. Housing 4602-A may be water-resistant. For example, housing 4602-A may withstand immersion in about one (1) meter of water for 30 minutes.

Sensor electronics 122 (herein also referred to as simply ‘electronics’) (not shown), such as described with respect to FIG. 1R-1, may be retained within housing 4602-A. Sensor electronics 122 may include processing electronics present on a semiconductor chip. Sensor electronics 122 may further include wireless communications electronics present on the semiconductor chip and configured to communicate according to a Bluetooth Low Energy protocol. Sensor electronics 122 may also include a coin cell battery as a power source 320 and a magnetic switch to activate some or all of the sensor electronics in response to a magnetic field. Sensor electronics 122 may be coupled to a printed circuit board.

On-body device 4601-A includes an adhesive patch 4605-A attached to the housing 4602-A and configured to attach the housing to skin of the user. Adhesive patch 4605-A may protrude beyond a peripheral boundary 4606-A of housing 4602-A as shown. Adhesive patch 4605-A can be configured in accordance with any of the embodiments of adhesive patches described herein, including those adhesive patch embodiments with one or more removable patch portions (as described herein in the section titled ‘REMOVABLE PATCH PORTIONS’), one or more non-isotropic layers (as described herein in the section titled ‘NON-ISOTROPIC PATCHES’) and/or variations (e.g., openings, polymeric layers, etc.) thereof, layered material constructions (e.g., such as described with respect to FIG. 45), or any combination thereof. Adhesive patch 4605-A can have a circular outer periphery as shown here, or a periphery with a shape corresponding to the outer periphery of housing 4602-A, or otherwise.

On body device 4601-A may include a temperature sensor configured to detect a temperature of the body of the on-body device wearer. The temperature sensor may be present on an underside of first part 4603-A of housing 4602-A as described with respect to FIGS. 1R-10 and IR-11.

On-body device 4601-A may include a sensor 161A, such as described with respect to the embodiments of FIGS. 1O-1, 1O-2, 1O-3, 1O-4, 1Q-1, and/or 1Q-2. Sensor 161A may have a flexible substrate and may include a first portion retained within the housing and including three contacts configured to be in electrical contact with the sensor electronics. Sensor 161A may include a second portion configured to be positioned in the body of the user for monitoring a level of an analyte of the user. The second portion may have a length and a width arranged in a plane and may have a thickness perpendicular to the plane as depicted in the end on view of FIG. 1O-5. The thickness may be smaller than the width. The second portion may include three electrodes connected to connections that are in turn connected to the three contacts of the first portion. Sensor 161A may have a fold such as described with respect to FIG. 1Q-2.

On-body device 4601-A can be further configured according to any of the embodiments described herein, including with one or more vent passageways (as described herein in the section titled ‘VENT PASSAGEWAY’), one or more vent seals (as described herein in the section titled ‘VENT SEAL’), one or more moisture egress channels (as described herein in the section titled ‘ADDITIONAL MOISTURE EGRESS FEATURES—Channels’), one or more surface textures (as described herein in the section titled ‘ADDITIONAL MOISTURE EGRESS FEATURES—Surface Texture’), one or more reinforcement devices (as described herein in the section titled ‘REINFORCEMENT DEVICES’), one or more anti-stick layers, or any combination thereof.

System 10-A can include an overpatch 4661-A configured to be applied over on-body device 4601-A and/or adhesive patch adhesive patch 4605-A. Overpatch 4661-A can be configured in accordance with any of the embodiments of adhesive overpatches described herein, including those adhesive patch embodiments with one or more removable patch portions, one or more non-isotropic layers and/or variations (e.g., openings, polymeric layers, etc.) thereof, layered material constructions (e.g., such as described with respect to FIG. 45), one or more support structures, one or more removable liners, or any combination thereof. In this embodiment, overpatch 4661-A has an inner periphery 4642-A about an opening that corresponds to the shape of the periphery of housing 4602-A of on-body device 4601-A. Overpatch 4661-A may have an outer periphery 4663-A having a shape that does or does not (as shown here) correspond to the shape of the periphery of housing 4602-A. Other shapes (e.g., circular, oval, asymmetric, polygonal or a combination thereof) for inner periphery 4662-A and outer periphery 4663-A may also be used.

On-body device 4601-A may be housed within an interior space of inserter 4621-A with the interior space being sealed by removable cap 4622-A coupled with handle 4623-A and/or another housing portion of inserter 4621-A. Cap 4622-A can be removed by manipulating a latch 4624-A (e.g., by lifting the latch) to permit removal. Cap 4622-A may include a sheath that covers the sensor and sharp and maintains them in a sterile environment (such as described with respect to FIGS. 1U-14 and 1U-15). Removal of cap 4622-A may remove the sheath and expose the sensor and sharp to the ambient environment.

Inserter 4621-A may include the handle 4623-A, a housing 4624-A, a sharp (not shown), and a retraction spring (not shown). Inserter 4621-A may be configured for manual advancement of the sharp with the handle 4623-A and automatic retraction of the sharp with the retraction spring, such as described with respect to FIGS. 1T-1, 1T-2, 1T-3, 1U-1, 1U-2, and/or 1U-3. Handle 4623-A can be configured to move from an initial position to an advanced position with respect to a housing (not shown in FIG. 46A) to advance the sharp into skin of a recipient's body and concurrently advance on-body device 4601-A into position on the recipient's body. Inserter 4621-A may be constructed and provided to an end user with only one coil spring for moving the sharp, where that coil spring is a retraction spring and not an advancement spring.

The sharp may be configured as described with respect to any of FIGS. 1U-23 through 1U-30. The sharp may include a distal tip portion with a V-shaped portion having an angle (e.g., angle 661) of 20 to 60 degrees and, in some embodiments, the angle can be between 45 and 55 degrees. The sharp may include a proximal shaft portion having a first sidewall, a floor, and a second sidewall with a U-shape or a C-shape cross-sectional profile. The sharp may include a transition portion between the distal tip portion and the proximal shaft portion where the first sidewall and the second sidewall each have a height from the floor that is sloped at an angle (e.g., angle 669) of 7 to 35 degrees and, in some embodiments, the angle is between 20 and 30 degrees.

Inserter 4621-A can be further configured according to any of the embodiments described herein.

Receiver unit 4641-A may be a dedicated receiver unit built for the on-body device manufacturer for the purpose of communicating with on-body device 4601-A. Receiver unit 4641-A may alternatively be a smart phone or a smart watch. Receiver unit 4641-A may include processing electronics communicatively coupled with memory as described with respect to FIG. 1R-13. Receiver unit 4641-A may include a software application stored thereon and for execution by the receiver unit (e.g., by the processing electronics). Receiver unit 4641-A may include a display 4645-A for outputting analyte values and other information to a user. Receiver unit 4641-A may be configured to receive analyte data associated with the analyte level of the user from on-body device 4601-A and display information corresponding to the analyte data to the user. Display 4645-A may be configured as a touchscreen permitting user input. Other tangible user input interfaces 4646-A can be included.

The software application may be configured to generate a present analyte value of the on-body device wearer (the sensor recipient), based on a measurement by the sensor, for output to the user. System 10-A can be configured, such as by configuration of on-body device 4601-A and/or the software application of receiver unit 4641-A, so that the analyte value of the wearer is not displayed before expiration of a warm-up period of the sensor. The warm-up period is a period of time after activation of the sensor where the sensor may adjust to the conditions of the human body and obtain a normal or optimal operating condition for obtaining accurate measurements. The warm up period may be 30 minutes or less, may be one hour, may be two hours, or may be longer depending on the design and construction of the sensor.

System 10-A can be configured, such as by configuration of on-body device 4601-A and/or software application 4644-A of receiver unit 4641-A, so that a mean average relative difference (MARD) of the analyte values displayed to the user are 9.5% or less in one embodiment, 9.0% or less in another embodiment, and/or 8.5% or less in another embodiment.

System 10-A can be configured, such as by configuration of on-body device 4601-A and/or software application 4644-A of receiver unit 4641-A, to apply a factory calibration to, in part, calculate an analyte value for display to the user. The factory calibration can be based on a measurement of the sensor made by the manufacturer, or based on a measurement of a comparable but different sensor made by the manufacturer, that is indicative of the sensing performance of the sensor. This measurement can be used to generate calibration information that is applied to measurements made by the actual sensor 161A provided as part of on-body device 4601-A. The factory calibration can be the only calibration that is applied by system 10-A (e.g., receiver unit 4641-A). System 10-A (e.g. receiver unit 4641-A) can be configured to not require calibration information directly from the user, such as a blood glucose value taken from a fingerstick measurement, prior to displaying the analyte value to the user that is based on a measurement made of the user by sensor 161A. Alternatively, system 10-A (e.g. receiver unit 4641-A) can be configured to accept calibration information directly from the user, such as a blood glucose value taken from a fingerstick measurement, and use that as a supplement to the factory calibration.

System 10-A and receiver unit 4641-A can be further configured according to any of the embodiments described herein.

### System 10-B

An example embodiment of a system 10-B is shown in the landscape view of FIG. 46B. Here, system 10-B includes on-body device 4601-B, inserter 4621-B, receiver unit 4641-B, and/or overpatch 4661-B. On-body device 4601-B can be configured in accordance with any of the embodiments of on-body device 100 described herein. On-body device 4601-A is shown in both top down and side views. Here, on-body device 4601-B includes a housing 4602-B which can be a molded housing, such as a housing having the characteristics of a one-shot molded housing. In this embodiment, the peripheral boundary 4606-B of housing 4602-B is configured like the embodiment described with respect to FIG. 1N-13. The shape of each side may be interrupted by features. For example, housing 4602-B may include one or more recesses adapted to interface with projections of inserter 4621-B, e.g., such as on deflectable projections, that are configured to releasably hold on-body device 4601-B within inserter 4621-B, for example, as described with respect to the embodiments of FIGS. 1U-16 through 1U-22. Here, housing 4602-B includes four recesses, with locations identified by reference numerals 4607-B-1, 4607-B-2, 4608-B-1, and 4608-B-2. Housing 4602-B includes a sharp aperture 4615-B and may include a recess 4616-B in a top surface of housing 4602-B configured to interface with a projection of inserter that together inhibit rotation of housing 4602-B during application of on-body device 4601-B to the body of the recipient. Housing 4602-B may be configured to be water-resistant. For example, housing 4602-B may withstand immersion in about one (1) meter of water for 30 minutes.

Sensor electronics 122 (herein also referred to as simply ‘electronics’) (not shown), such as described with respect to FIG. 1R-1, may be retained within housing 4602-B. Sensor electronics 122 may include sensor interface electronics and wireless communications electronics configured to communicate according to a Bluetooth Low Energy protocol. Sensor electronics 122 may include a first antenna configured for communication according to the Bluetooth Low Energy protocol. Sensor electronics 122 may include a second antenna configured for communication according to a Near Field Communication protocol, that may be used during manufacturing for storing data and/or testing on-body device 4601-B. Sensor electronics 122 may include a coin cell battery as a power source 320 and a magnetic switch to activate some or all of the sensor electronics in response to a magnetic field. Sensor electronics 122 may have the sensor interface electronics present on a first semiconductor chip, such as an application specific integrated circuit (ASIC). Sensor electronics 122 may have the wireless communication electronics present on a second, different, semiconductor chip. Sensor electronics 122 may be coupled to a printed circuit board.

On-body device 4601-B includes an adhesive patch 4605-B attached to housing 4602-B and configured to attach housing 4602-B to skin of the user. Adhesive patch 4605-B may protrude beyond a peripheral boundary 4606-B of housing 4602-B as shown. Adhesive patch 4605-B can be configured in accordance with any of the embodiments of adhesive patches described herein, including those adhesive patch embodiments with one or more removable patch portions (as described herein in the section titled ‘REMOVABLE PATCH PORTIONS’), one or more non-isotropic layers (as described herein in the section titled ‘NON-ISOTROPIC PATCHES’) and/or variations (e.g., openings, polymeric layers, etc.) thereof, layered material constructions (e.g., such as described with respect to FIG. 45), or any combination thereof. Adhesive patch 4605-B can have a periphery with a shape corresponding to the outer periphery of housing 4602-B as shown here, or a circular or elliptical outer periphery, or otherwise.

On-body device 4601-B may include a sensor 161B, such as described with respect to the embodiments of FIGS. 1P-1, 1P-2, and/or 1P-3. Sensor 161B may be configured to detect or measure an analyte with a working electrode and a counter/reference electrode. Sensor 161B may include a central substrate configured as a wire. Sensor 161B may include a plurality of layers on the central substrate, the plurality of layers including a diffusion limiting layer, an enzyme layer, and an interferent layer. Other layers may be present. Sensor 161B may have a bend such as described with respect to FIGS. 1P-1 and 1P-2.

On-body device 4601-B can be further configured according to any of the embodiments described herein, including with one or more vent passageways (as described herein in the section titled ‘VENT PASSAGEWAY’), one or more vent seals (as described herein in the section titled ‘VENT SEAL’), one or more moisture egress channels (as described herein in the section titled ‘ADDITIONAL MOISTURE EGRESS FEATURES-Channels’), one or more surface textures (as described herein in the section titled ‘ADDITIONAL MOISTURE EGRESS FEATURES-Surface Texture’), one or more reinforcement devices (as described herein in the section titled ‘REINFORCEMENT DEVICES’), one or more anti-stick layers, or any combination thereof.

System 10-B can include an overpatch 4661-B configured to be applied over on-body device 4601-B and/or adhesive patch adhesive patch 4605-B. Overpatch 4661-B can be configured in accordance with any of the embodiments of adhesive overpatches described herein, including those adhesive patch embodiments with one or more removable patch portions, one or more non-isotropic layers and/or variations (e.g., openings, polymeric layers, etc.) thereof, layered material constructions (e.g., such as described with respect to FIG. 45), one or more support structures, one or more removable liners, or any combination thereof. In this embodiment, overpatch 4661-B has an inner periphery 4642-B about an opening that corresponds to the shape of the periphery of housing 4602-B of on-body device 4601-B. Overpatch 4661-B may have an outer periphery 4663-B having a shape that does or does not (as shown here) correspond to the shape of the periphery of housing 4602-B. Other shapes (e.g., circular, oval, asymmetric, polygonal or a combination thereof) for inner periphery 4662-B and outer periphery 4663-B may also be used.

On-body device 4601-B may be pre-loaded and housed within an interior space of inserter 4621-B. Inserter 4621-B may include a housing 4624-B having an actuator 4625-B configured to initiate advancement of the sharp. The actuator 4625-B may be configured as a push button. Inserter 4621-B may include a sharp, a first spring configured to advance the sharp, and a second spring configured to retract the sharp into housing 4624-B. The first spring may be a component of an insertion mechanism and the second spring may be a component of a retraction mechanism as described herein. The interior space of inserter 4621-B may be sealed by a removable cap 4622-B coupled with housing 4624-B. Inserter 4621-B may be as described with respect to FIGS. 1T-4, 1T-5, 1T-6, 1U-4, 1U-5, and/or 1U-6 and configured to automatically advance a sharp and move on-body device 4601-B within inserter 4621-B, and then to automatically retract the sharp.

The sharp may be configured as described with respect to any of FIGS. 1U-23 through 1U-30. The sharp may include a shaft comprising a first sidewall, a floor, and a second sidewall together providing a U-shape or a C-shape cross-sectional profile. The shaft may have a bevelled distal tip with an angle of between 15 and 55 degrees, or more preferably between 20 and 45 degrees.

Inserter 4621-B can be further configured according to any of the embodiments described herein.

Receiver unit 4641-B may be a dedicated receiver unit built for the on-body device manufacturer for the purpose of communicating with on-body device 4601-B. Receiver unit 4641-B may alternatively be a smart phone or a smart watch. Receiver unit 4641-B may include processing electronics communicatively coupled with memory as described with respect to FIG. 1R-13. Receiver unit 4641-B may include a software application stored thereon and for execution by the receiver unit (e.g., by the processing electronics). Receiver unit 4641-B may include a display 4645-B for outputting analyte values and other information to a user. Display 4645-B may be configured as a touchscreen permitting user input. Other tangible user input interfaces 4646-B can be included. Receiver unit 4641-B may be configured to receive analyte data associated with the analyte level of the user from on-body device 4601-B and display information corresponding to the analyte data to the user.

The software application may be configured to generate a present analyte value of the on-body device wearer (the sensor recipient), based on a measurement by the sensor, for output to the user. System 10-B can be configured, such as by configuration of on-body device 4601-B and/or the software application of receiver unit 4641-B, so that the analyte value of the wearer is not displayed before expiration of a warm-up period of the sensor, as described with respect to system 10-B.

System 10-B can be configured, such as by configuration of on-body device 4601-B and/or the software application of receiver unit 4641-B, so that a mean average relative difference (MARD) of the analyte values displayed to the user are 9.5% or less in one embodiment, 9.0% or less in another embodiment, and/or 8.5% or less in another embodiment.

System 10-B can be configured, such as by configuration of on-body device 4601-B and/or the software application of receiver unit 4641-B, to apply a factory calibration to, in part, calculate an analyte value for display to the user. The factory calibration can be based on a measurement of the sensor made by the manufacturer or based on a measurement of a comparable but different sensor made by the manufacturer, that is indicative of the sensing performance of the sensor. This measurement can be used to generate calibration information that is applied to measurements made by the actual sensor 161B provided as part of on-body device 4601-B. The factory calibration can be the only calibration that is applied by system 10-B (e.g., receiver unit 4641-B). System 10-B (e.g. receiver unit 4641-B) can be configured to not require calibration information directly from the user, such as a blood glucose value taken from a fingerstick measurement, prior to displaying the analyte value to the user that is based on a measurement made of the user by sensor 161B. Alternatively, system 10-B (e.g. receiver unit 4641-B) can be configured to accept calibration information directly from the user, such as a blood glucose value taken from a fingerstick measurement, and use that as a supplement to the factory calibration.

System 10-B and receiver unit 4641-B can be further configured according to any of the embodiments described herein.

### System 10-C

An example embodiment of a system 10-C is shown in the landscape view of FIG. 46C. Here, system 10-C includes on-body device 4601-C, inserter 4621-C, receiver unit 4641-C, and/or overpatch 4661-C. On-body device 4601-C can be configured in accordance with any of the embodiments of on-body device 100 described herein. On-body device 4601-A is shown in both top down and side views. Here, on-body device 4601-C includes a housing 4602-C having a first part 4603-C and a second part 4604-C attached together. In this embodiment, the peripheral boundary 4606-C of housing 4602-C is configured like the embodiment described with respect to FIG. 1B-3, with a circular boundary side. The circular shape may be interrupted by features. For example, housing 4602-C may include one or more recesses adapted to interface with projections of inserter 4621-C, e.g., such as deflectable projections, that are configured to releasably hold on-body device 4601-C within inserter 4621-C, for example, as described with respect to the embodiments of FIGS. 1U-16 through 1U-22. Here, housing 4602-C includes such two recesses 4607-C and 4608-C. Housing 4602-C includes a sharp aperture 4615-C. Housing 4602-C may be water-resistant. For example, housing 4602-C may withstand immersion in about one (1) meter of water for 30 minutes.

Sensor electronics 122 (not shown), such as described with respect to FIG. 1R-1, may be retained within housing 4602-C. Sensor electronics 122 may include processing electronics present on a semiconductor chip. Sensor electronics 122 may further include wireless communications electronics present on the semiconductor chip and configured to communicate according to a Bluetooth Low Energy protocol. Sensor electronics 122 may also include a coin cell battery as a power source 320 and a magnetic switch to activate some or all of the sensor electronics in response to a magnetic field. Some of sensor electronics 122 may be coupled to a printed circuit board. Sensor electronics 122 may include an antenna configured to communicate according to the Bluetooth Low Energy protocol. The antenna may be mounted on the housing and electrically connected to the printed circuit board, as described with respect to FIG. 1R-12. The coin cell battery 320 may be electrically connected to the printed circuit board and held adjacent to the printed circuit board such that the coin cell battery and the printed circuit board are both in a same plane as described with respect to FIGS. 1R-7, 1R-8, and/or 1R-9.

On-body device 4601-C includes an adhesive patch 4605-C attached to the housing 4602-C and configured to attach the housing to skin of the user. Adhesive patch 4605-C may protrude beyond a peripheral boundary 4606-C of housing 4602-C as shown. Adhesive patch 4605-C can be configured in accordance with any of the embodiments of adhesive patches described herein, including those adhesive patch embodiments with one or more removable patch portions (as described herein in the section titled ‘REMOVABLE PATCH PORTIONS’), one or more non-isotropic layers (as described herein in the section titled ‘NON-ISOTROPIC PATCHES’) and/or variations (e.g., openings, polymeric layers, etc.) thereof, layered material constructions (e.g., such as described with respect to FIG. 45), or any combination thereof. Adhesive patch 4605-D can have a circular outer periphery as shown here, or a periphery with a shape not corresponding to the outer periphery of housing 4602-D, or otherwise.

On-body device 4601-C may include a sensor 161A, such as described with respect to the embodiments of FIGS. 1O-1, 1O-2, 1O-3, 1O-4, 1Q-1, and/or 1Q-2. Sensor 161A may have a flexible substrate and may include a first portion retained within the housing and including three contacts configured to be in electrical contact with the sensor electronics. Sensor 161A may include a second portion configured to be positioned in the body of the user for monitoring a level of an analyte of the user. The second portion may have a length and a width arranged in a plane and may have a thickness perpendicular to the plane as depicted in the end on view of FIG. 1O-5. The thickness may be smaller than the width. The second portion may include three electrodes connected to connections that are in turn connected to the three contacts of the first portion. Sensor 161A may have a fold such as described with respect to FIG. 1Q-2. Alternatively, sensor 161A may not have a fold, such as described with respect to FIG. 1Q-1.

On-body device 4601-C can be further configured according to any of the embodiments described herein, including with one or more vent passageways (as described herein in the section titled ‘VENT PASSAGEWAY’), one or more vent seals (as described herein in the section titled ‘VENT SEAL’), one or more moisture egress channels (as described herein in the section titled ‘ADDITIONAL MOISTURE EGRESS FEATURES—Channels’), one or more surface textures (as described herein in the section titled ‘ADDITIONAL MOISTURE EGRESS FEATURES—Surface Texture’), one or more reinforcement devices (as described herein in the section titled ‘REINFORCEMENT DEVICES’), one or more anti-stick layers, or any combination thereof.

System 10-C can include an overpatch 4661-C configured to be applied over on-body device 4601-C and/or adhesive patch adhesive patch 4605-C. Overpatch 4661-C can be configured in accordance with any of the embodiments of adhesive overpatches described herein, including those adhesive patch embodiments with one or more removable patch portions, one or more non-isotropic layers and/or variations (e.g., openings, polymeric layers, etc.) thereof, layered material constructions (e.g., such as described with respect to FIG. 45), one or more support structures, one or more removable liners, or any combination thereof. In this embodiment, overpatch 4661-C has a circular inner periphery 4642-C about an opening that corresponds to the shape of the circular periphery of housing 4602-C of on-body device 4601-C. Overpatch 4661-C may have an outer periphery 4663-C having a shape that does (as shown here) or does not correspond to the shape of the periphery of housing 4602-C. Other shapes (e.g., oval, asymmetric, polygonal or a combination thereof) for inner periphery 4662-C and outer periphery 4663-C may also be used.

On-body device 4601-C may be housed within an interior space of inserter 4621-C with the interior space being sealed by removable cap 4622-C coupled with handle 4623-C and/or housing 4624-C of inserter 4621-C. Cap 4622-C can be removed by manipulating a latch 4624-C (e.g., by lifting the latch) to permit removal. Cap 4622-C may include a sheath that covers the sensor and sharp and maintains them in a sterile environment (such as described with respect to FIGS. 1U-14 and 1U-15). Removal of cap 4622-C may remove the sheath and expose the sensor and sharp to the ambient environment.

On-body device 4601-C may be pre-loaded and housed within an interior space of inserter 4621-C. Inserter 4621-C may include a housing 4624-C having an actuator 4625-C configured to initiate advancement of the sharp. The actuator 4625-C may be configured as a push button. Inserter 4621-C may include a sharp, a first spring configured to advance the sharp, and a second spring configured to retract the sharp into housing 4624-C. The first spring may be a component of an insertion mechanism and the second spring may be a component of a retraction mechanism as described herein. The interior space of inserter 4621-C may be sealed by a removable cap 4622-C coupled with housing 4624-C. Inserter 4621-C may be as described with respect to FIGS. 1T-4, 1T-5, 1T-6, 1U-4, 1U-5, and/or 1U-6 and configured to automatically advance a sharp and move on-body device 4601-C within inserter 4621-C, and then to automatically retract the sharp.

The sharp may be configured as described with respect to any of FIGS. 1U-23 through 1U-30. The sharp may include a distal tip portion with a V-shaped portion having an angle (e.g., angle 661) of 20 to 60 degrees and, in some embodiments, the angle can be between 28 and 38 degrees. The sharp may include a proximal shaft portion having a first sidewall, a floor, and a second sidewall with a U-shape or a C-shape cross-sectional profile. The sharp may include a transition portion between the distal tip portion and the proximal shaft portion where the first sidewall and the second sidewall each have a height from the floor that is sloped at an angle (e.g., angle 669) of 7 to 35 degrees and, in some embodiments, the angle is between 18 and 28 degrees. Sharp aperture 4615-C permits passage of the sharp through housing 4602-C. Inserter 4621-C can be further configured according to any of the embodiments described herein.

Receiver unit 4641-C may be a dedicated receiver unit built for the on-body device manufacturer for the purpose of communicating with on-body device 4601-C. Receiver unit 4641-C may alternatively be a smart phone or a smart watch. Receiver unit 4641-C may include processing electronics communicatively coupled with memory as described with respect to FIG. 1R-13. Receiver unit 4641-C may include a software application stored thereon and for execution by the receiver unit (e.g., by the processing electronics). Receiver unit 4641-C may include a display 4645-C for outputting analyte values and other information to a user. Receiver unit 4641-C may be configured to receive analyte data associated with the analyte level of the user from on-body device 4601-C and display information corresponding to the analyte data to the user. Display 4645-C may be configured as a touchscreen permitting user input. Other tangible user input interfaces 4646-C can be included.

The software application may be configured to generate a present analyte value of the on-body device wearer (the sensor recipient), based on a measurement by the sensor, for output to the user. System 10-C can be configured, such as by configuration of on-body device 4601-C and/or the software application of receiver unit 4641-C, so that the analyte value of the wearer is not displayed before expiration of a warm-up period of the sensor. The warm-up period is a period of time after activation of the sensor where the sensor may adjust to the conditions of the human body and obtain a normal or optimal operating condition for obtaining accurate measurements. The warm up period may be 30 minutes or less, may be one hour, may be two hours, or may be longer depending on the design and construction of the sensor.

System 10-C can be configured, such as by configuration of on-body device 4601-C and/or the software application of receiver unit 4641-C, so that a mean average relative difference (MARD) of the analyte values displayed to the user are 9.5% or less in one embodiment, 9.0% or less in another embodiment, and/or 8.5% or less in another embodiment.

System 10-C can be configured, such as by configuration of on-body device 4601-C and/or the software application of receiver unit 4641-C, to apply a factory calibration to, in part, calculate an analyte value for display to the user, as described with respect to system 10-A.

System 10-C and receiver unit 4641-C can be further configured according to any of the embodiments described herein.

### System 10-D

An example embodiment of a system 10-D is shown in the landscape view of FIG. 46D. Here, system 10-D includes on-body device 4601-D, inserter 4621-D, receiver unit 4641-D, and/or overpatch 4661-D. On-body device 4601-D can be configured in accordance with any of the embodiments of on-body device 100 described herein. On-body device 4601-A is shown in both top down and side views. Here, on-body device 4601-D includes a housing 4602-D having a first part 4603-D and a second part 4604-D attached together. In this embodiment, the peripheral boundary 4606-D of housing 4602-D is configured like the embodiment described with respect to FIG. 1B-3, with a circular boundary side. In other embodiments the peripheral boundary 4606-D of housing 4602-D is octagonal, like the embodiment described with respect to FIG. 1N-19. The octagonal or circular shape may be interrupted by features. For example, housing 4602-D may include two or more recesses 4607-D and 4608-D adapted to interface with projections of inserter 4621-D, e.g., such as deflectable projections, that are configured to releasably hold on-body device 4601-D, for example, as described with respect to the embodiments of FIGS. 1U-16 through 1U-22. Housing 4602-D may include a recess 4609-D configured to receive a sensor connector 4610-D. Sensor connector 4610-D, which can be considered part of housing 4602-D when mated thereto, includes a sharp aperture 4615-D and engagement apertures 4631-D and 4632-D for engaging with and permitting releasable holding by an advanceable holder portion (not shown) of inserter 4621-D within inserter housing 4624-D. The holder portion (not shown) can have, for example, deflectable arms with detents that are insertable into complementary receiving portions of engagement apertures 4631-D and 4632-D and releasable from apertures 4631-D and 4632-D after sensor connector 4610-D has been coupled with housing 4602-D. Housing 4602-D may be water-resistant when the sensor connector 4610-D is secured within recess 4609-D. For example, housing 4602-D may withstand immersion in about one (1) meter of water for 30 minutes.

Sensor electronics 122 (not shown), such as described with respect to FIG. 1R-1, may be retained within housing 4602-D. Sensor electronics 122 may include processing electronics present on a semiconductor chip. Sensor electronics 122 may further include wireless communications electronics present on the semiconductor chip and configured to communicate according to a Bluetooth Low Energy protocol. Sensor electronics 122 may also include a coin cell battery as a power source 320 and a magnetic switch to activate some or all of the sensor electronics in response to a magnetic field. Some of sensor electronics 122 may be coupled to a printed circuit board. Sensor electronics 122 may include an antenna configured to communicate according to the Bluetooth Low Energy protocol. The coin cell battery 320 may be electrically connected to the printed circuit board and held adjacent to the printed circuit board such that the coin cell battery and the printed circuit board are both in a same plane as described with respect to FIGS. 1R-7, 1R-8, and/or 1R-9.

On-body device 4601-D includes an adhesive patch 4605-D attached to the housing 4602-D and configured to attach the housing to skin of the user. Adhesive patch 4605-D may protrude beyond a peripheral boundary 4606-D of housing 4602-D as shown. Adhesive patch 4605-D can be configured in accordance with any of the embodiments of adhesive patches described herein, including those adhesive patch embodiments with one or more removable patch portions (as described herein in the section titled ‘REMOVABLE PATCH PORTIONS’), one or more non-isotropic layers (as described herein in the section titled ‘NON-ISOTROPIC PATCHES’) and/or variations (e.g., openings, polymeric layers, etc.) thereof, layered material constructions (e.g., such as described with respect to FIG. 45), or any combination thereof. Adhesive patch 4605-D can have a circular outer periphery as shown here, or a periphery with a shape not corresponding to the outer periphery of housing 4602-D, or otherwise.

On-body device 4601-D may include a sensor 161A, such as described with respect to the embodiments of FIGS. 1O-1, 1O-2, 1O-3, 1O-4, 1Q-1, and/or 1Q-2. Sensor 161A may be coupled to the sensor connector 4610-D. Sensor 161A may have a flexible substrate and may include a first portion retained within the housing and including three contacts configured to be in electrical contact with the sensor electronics. Sensor 161A may include a second portion configured to be positioned in the body of the user for monitoring a level of an analyte of the user. The second portion may have a length and a width arranged in a plane and may have a thickness perpendicular to the plane as depicted in the end on view of FIG. 1O-5. The thickness may be smaller than the width. The second portion may include three electrodes connected to connections that are in turn connected to the three contacts of the first portion. Sensor 161A may have a fold such as described with respect to FIG. 1Q-2. Alternatively, sensor 161A may not have a fold, such as described with respect to FIG. 1Q-1.

On-body device 4601-D can be further configured according to any of the embodiments described herein, including with one or more vent passageways (as described herein in the section titled ‘VENT PASSAGEWAY’), one or more vent seals (as described herein in the section titled ‘VENT SEAL’), one or more moisture egress channels (as described herein in the section titled ‘ADDITIONAL MOISTURE EGRESS FEATURES—Channels’), one or more surface textures (as described herein in the section titled ‘ADDITIONAL MOISTURE EGRESS FEATURES—Surface Texture’), one or more reinforcement devices (as described herein in the section titled ‘REINFORCEMENT DEVICES’), one or more anti-stick layers, or any combination thereof.

System 10-D can include an overpatch 4661-D configured to be applied over on-body device 4601-D and/or adhesive patch adhesive patch 4605-D. Overpatch 4661-D can be configured in accordance with any of the embodiments of adhesive overpatches described herein, including those adhesive patch embodiments with one or more removable patch portions, one or more non-isotropic layers and/or variations (e.g., openings, polymeric layers, etc.) thereof, layered material constructions (e.g., such as described with respect to FIG. 45), one or more support structures, one or more removable liners, or any combination thereof. In this embodiment, overpatch 4661-D has a circular inner periphery 4642-D about an opening that corresponds to the shape of the periphery of housing 4602-D of on-body device 4601-D. Overpatch 4661-D may have an outer periphery 4663-D having a shape that does (circular, as shown here) or does not correspond to the shape of the periphery of housing 4602-D. Other shapes (e.g., oval, asymmetric, polygonal or a combination thereof) for inner periphery 4662-D and outer periphery 4663-D may also be used.

On-body device 4601-D may be provided to an end user in a tray 4626-D. On-body device housing 4602-D may include one or more features 4617-D, such as a recess, indentation, or aperture, that assists interfacing with a complementary structure in tray 4626-D to retain housing 4602-D in tray 4626-D. Inserter 4621-D may be provided to the end user with sensor connector (having sensor 161A coupled thereto) and a sharp maintained in a sterile state within an interior space of inserter 4621-D. Inserter 4621-D may include a housing 4624-D having an actuator 4625-D. Inserter housing 4624-D may include an alignment feature 4633-D that mechanically interfaces with a complementary feature 4634-D on or within tray 4626-D to ensure inserter housing 4624-D is properly aligned with housing 4602-D of on-body device 4601-D when inserter housing 4624-D is inserted into tray 4626-D. Inserter housing 4624-D may be mated with the on-body device 4601-D while in tray 4626-D to grasp the on-body device 4601-D and hold it at a distal end of the housing 4624-D, similar to the embodiment described with respect to FIGS. 1U-11 and 1U-12. The mating process may result in insertion of sensor connector 4610-D into recess 46009-D, or the insertion of sensor connector 4610_D can occur during the process of advancing the sharp as described below.

Removal of housing 4624-D from tray 4626-D removes on-body device 4601-D from tray and may also remove a liner from adhesive patch 4205-D, which may be retained in tray 4626-D. The actuator 4625-D may be configured to initiate advancement of the sharp and sensor connector such that the sharp penetrates the body and concurrently places the sensor in the penetration and connects the sensor connector 4610-D within recess 4609-D of the on-body housing, as described with respect to FIGS. 1U-11 and 1U-12. The actuator 4625-D may be configured as a push button. Inserter 4621-D may include a locking mechanism 4627-D that may be moved to an unlocked position that permits actuator to trigger insertion. Inserter 4621-D may include the sharp, a first spring configured to advance the sharp, and a second spring configured to retract the sharp into housing 4624-D. The first spring may be a component of an insertion mechanism and the second spring may be a component of a retraction mechanism as described herein. Inserter 4621-D may be as described with respect to FIGS. 1U-11 and 1U-12 and configured to automatically advance the sharp and sensor connector and then to automatically retract the sharp.

The sharp may be configured as described with respect to any of FIGS. 1U-23 through 1U-30. The sharp may include a distal tip portion with a V-shaped portion having an angle (e.g., angle 661) of 20 to 60 degrees and, in some embodiments, the angle can be between 30 and 40 degrees. The sharp may include a proximal shaft portion having a first sidewall, a floor, and a second sidewall with a U-shape or a C-shape cross-sectional profile. The sharp may include a transition portion between the distal tip portion and the proximal shaft portion where the first sidewall and the second sidewall each have a height from the floor that is sloped at an angle (e.g., angle 669) of 7 to 35 degrees and, in some embodiments, the angle is between 13 and 23 degrees. Sharp aperture 4615-D is for passage of the sharp through housing 4602-D. Inserter 4621-D can be further configured according to any of the embodiments described herein.

Receiver unit 4641-D may be a dedicated receiver unit built for the on-body device manufacturer for the purpose of communicating with on-body device 4601-D. Receiver unit 4641-D may alternatively be a smart phone or a smart watch. Receiver unit 4641-D may include processing electronics communicatively coupled with memory as described with respect to FIG. 1R-13. Receiver unit 4641-D may include a software application stored thereon and for execution by the receiver unit (e.g., by the processing electronics). Receiver unit 4641-D may include a display 4645-D for outputting analyte values and other information to a user. Receiver unit 4641-D may be configured to receive analyte data associated with the analyte level of the user from on-body device 4601-D and display information corresponding to the analyte data to the user. Display 4645-D may be configured as a touchscreen permitting user input. Other tangible user input interfaces 4646-D can be included.

The software application may be configured to generate a present analyte value of the on-body device wearer (the sensor recipient), based on a measurement by the sensor, for output to the user. System 10-D can be configured, such as by configuration of on-body device 4601-D and/or the software application of receiver unit 4641-D, so that the analyte value of the wearer is not displayed before expiration of a warm-up period of the sensor. The warm-up period is a period of time after activation of the sensor where the sensor may adjust to the conditions of the human body and obtain a normal or optimal operating condition for obtaining accurate measurements. The warm up period may be 30 minutes or less, may be one hour, may be two hours, or may be longer depending on the design and construction of the sensor.

System 10-D can be configured, such as by configuration of on-body device 4601-D and/or the software application of receiver unit 4641-D, so that a mean average relative difference (MARD) of the analyte values displayed to the user are 9.5% or less in one embodiment, 9.0% or less in another embodiment, and/or 8.5% or less in another embodiment.

System 10-D can be configured, such as by configuration of on-body device 4601-D and/or the software application of receiver unit 4641-D, to apply a factory calibration to, in part, calculate an analyte value for display to the user, as described with respect to system 10-A.

System 10-D and receiver unit 4641-D can be further configured according to any of the embodiments described herein.

### System 10-E

An example embodiment of a system 10-E is shown in the landscape view of FIG. 46E. Here, system 10-E includes on-body device 4601-E, inserter 4621-E, receiver unit 4641-E, and/or overpatch 4661-E. On-body device 4601-E can be configured in accordance with any of the embodiments of on-body device 100 described herein. On-body device 4601-A is shown in both top down and side views. Here, on-body device 4601-E includes a housing 4602-E having a first part 4603-E and a second part 4604-E attached together. In this embodiment, the peripheral boundary 4606-E of housing 4602-E is octagonal, like the embodiment described with respect to FIG. 1N-19. In other embodiments boundary 4606-E may be circular like the embodiment described with respect to FIG. 1B-3. The octagonal or circular shape may be interrupted by features. For example, housing 4602-E may include at least one recess adapted to interface with a projection of inserter 4621-E, e.g., such as a rim or other snap projection, that is configured to releasably hold on-body device 4601-E, for example, as described with respect to any of the embodiments of FIGS. 1U-16 through 1U-22. Housing 4602-E may include a latch mechanism 4618-E accessible on an upper surface of housing 4602-E to permit housing 4602-E to be held by the inserter 4621-E, as described with respect to FIGS. 1U-20 through 1U-22. The latch mechanism 4618-E may be located about a sharp aperture 4615-E of housing 4602-E. Housing 4602-E may be water-resistant. For example, housing 4602-E may withstand immersion in about one (1) meter of water for 30 minutes.

Sensor electronics 122 (not shown), such as described with respect to FIG. 1R-1, may be retained within housing 4602-E. Sensor electronics 122 may include processing electronics present on a semiconductor chip. Sensor electronics 122 may further include wireless communications electronics present on the semiconductor chip and configured to communicate according to a Bluetooth Low Energy protocol. Sensor electronics 122 may also include a coin cell battery as a power source 320 and a magnetic switch to activate some or all of the sensor electronics in response to a magnetic field. Some of sensor electronics 122 may be coupled to a printed circuit board. Sensor electronics 122 may include an antenna configured to communicate according to the Bluetooth Low Energy protocol. The coin cell battery 320 may be electrically connected to the printed circuit board and held adjacent to the printed circuit board such that the coin cell battery and the printed circuit board are both in a same plane as described with respect to FIGS. 1R-7, 1R-8, and/or 1R-9.

On-body device 4601-E includes an adhesive patch 4605-E attached to the housing 4602-E and configured to attach the housing to skin of the user. Adhesive patch 4605-E may protrude beyond a peripheral boundary 4606-E of housing 4602-E as shown. Adhesive patch 4605-E can be configured in accordance with any of the embodiments of adhesive patches described herein, including those adhesive patch embodiments with one or more removable patch portions (as described herein in the section titled ‘REMOVABLE PATCH PORTIONS’), one or more non-isotropic layers (as described herein in the section titled ‘NON-ISOTROPIC PATCHES’) and/or variations (e.g., openings, polymeric layers, etc.) thereof, layered material constructions (e.g., such as described with respect to FIG. 45), or any combination thereof. Adhesive patch 4605-E can have a circular outer periphery as shown here, or a periphery with a shape corresponding to the outer periphery of housing 4602-E, or otherwise.

On-body device 4601-E may include a sensor 161A, such as described with respect to the embodiments of FIGS. 1O-1, 1O-2, 1O-3, 1O-4, 1Q-1, and/or 1Q-2. Sensor 161A may have a flexible substrate and may include a first portion retained within the housing and including three contacts configured to be in electrical contact with the sensor electronics. Sensor 161A may include a second portion configured to be positioned in the body of the user for monitoring a level of an analyte of the user. The second portion may have a length and a width arranged in a plane and may have a thickness perpendicular to the plane as depicted in the end on view of FIG. 1O-5. The thickness may be smaller than the width. The second portion may include three electrodes connected to connections that are in turn connected to the three contacts of the first portion. Sensor 161A may have a fold such as described with respect to FIG. 1Q-2. Alternatively, sensor 161A may not have a fold, such as described with respect to FIG. 1Q-1.

On-body device 4601-E can be further configured according to any of the embodiments described herein, including with one or more vent passageways (as described herein in the section titled ‘VENT PASSAGEWAY’), one or more vent seals (as described herein in the section titled ‘VENT SEAL’), one or more moisture egress channels (as described herein in the section titled ‘ADDITIONAL MOISTURE EGRESS FEATURES—Channels’), one or more surface textures (as described herein in the section titled ‘ADDITIONAL MOISTURE EGRESS FEATURES—Surface Texture’), one or more reinforcement devices (as described herein in the section titled ‘REINFORCEMENT DEVICES’), one or more anti-stick layers, or any combination thereof.

System 10-E can include an overpatch 4661-E configured to be applied over on-body device 4601-E and/or adhesive patch adhesive patch 4605-E. Overpatch 4661-E can be configured in accordance with any of the embodiments of adhesive overpatches described herein, including those adhesive patch embodiments with one or more removable patch portions, one or more non-isotropic layers and/or variations (e.g., openings, polymeric layers, etc.) thereof, layered material constructions (e.g., such as described with respect to FIG. 45), one or more support structures, one or more removable liners, or any combination thereof. In this embodiment, overpatch 4661-E has an inner periphery 4642-E about an opening that corresponds to the shape of the periphery of housing 4602-E of on-body device 4601-E. Overpatch 4661-E may have an outer periphery 4663-E having a shape that does or does not (as shown here) correspond to the shape of the periphery of housing 4602-E. Other shapes (e.g., circular, oval, asymmetric, polygonal or a combination thereof) for inner periphery 4662-E and outer periphery 4663-E may also be used.

On-body device 4601-E may be housed within an interior space of inserter 4621-E with the interior space being sealed by removable cap 4622-E coupled with housing 4624-E of inserter 4621-E. Cap 4622-E can be removed by twisting to release from the housing 4624-E. Cap 4622-E may include a sheath that covers the sensor and sharp and maintains them in a sterile environment (such as described with respect to FIGS. 1U-14 and 1U-15). Removal of cap 4622-E may remove the sheath and expose the sensor and sharp to the ambient environment.

On-body device 4601-E may be pre-loaded and housed within an interior space of inserter 4621-E. Inserter 4621-E may include a housing 4624-E having an actuator 4625-E configured to initiate advancement of the sharp. The actuator 4625-E may be configured as a push button. Inserter 4621-E may include a sharp, a first spring configured to advance the sharp, and a second spring configured to retract the sharp into housing 4624-E. The first spring may be a component of an insertion mechanism and the second spring may be a component of a retraction mechanism as described herein. The interior space of inserter 4621-E may be sealed by a removable cap 4622-E coupled with housing 4624-E. Inserter 4621-E may be as described with respect to FIGS. 1T-4, 1T-5, 1T-6, 1U-4, 1U-5, and/or 1U-6 and configured to automatically advance a sharp and move on-body device 4601-E within inserter 4621-E, and then to automatically retract the sharp.

The sharp may be configured as described with respect to any of FIGS. 1U-23 through 1U-30. The sharp may include a distal tip portion with a V-shaped portion having an angle (e.g., angle 661) of 20 to 60 degrees and, in some embodiments, the angle can be between 31 and 41 degrees. The sharp may include a proximal shaft portion having a first sidewall, a floor, and a second sidewall with a U-shape or a C-shape cross-sectional profile. The sharp may include a transition portion between the distal tip portion and the proximal shaft portion where the first sidewall and the second sidewall each have a height from the floor that is sloped at an angle (e.g., angle 669) of 7 to 35 degrees and, in some embodiments, the angle is between 13 and 23 degrees. On-body device 4601-E may include the sharp aperture 4615-E for passage of the sharp through housing 4602-E.

Inserter 4621-E can be further configured according to any of the embodiments described herein.

Receiver unit 4641-E may be a dedicated receiver unit built for the on-body device manufacturer for the purpose of communicating with on-body device 4601-E. Receiver unit 4641-E may alternatively be a smart phone or a smart watch. Receiver unit 4641-E may include processing electronics communicatively coupled with memory as described with respect to FIG. 1R-13. Receiver unit 4641-E may include a software application stored thereon and for execution by the receiver unit (e.g., by the processing electronics). Receiver unit 4641-E may include a display 4645-E for outputting analyte values and other information to a user. Receiver unit 4641-E may be configured to receive analyte data associated with the analyte level of the user from on-body device 4601-E and display information corresponding to the analyte data to the user. Display 4645-E may be configured as a touchscreen permitting user input. Other tangible user input interfaces 4646-E can be included.

The software application may be configured to generate a present analyte value of the on-body device wearer (the sensor recipient), based on a measurement by the sensor, for output to the user. System 10-E can be configured, such as by configuration of on-body device 4601-E and/or the software application of receiver unit 4641-E, so that the analyte value of the wearer is not displayed before expiration of a warm-up period of the sensor. The warm-up period is a period of time after activation of the sensor where the sensor may adjust to the conditions of the human body and obtain a normal or optimal operating condition for obtaining accurate measurements. The warm up period may be 30 minutes or less, may be one hour, may be two hours, or may be longer depending on the design and construction of the sensor.

System 10-E can be configured, such as by configuration of on-body device 4601-E and/or the software application of receiver unit 4641-E, so that a mean average relative difference (MARD) of the analyte values displayed to the user are 9.5% or less in one embodiment, 9.0% or less in another embodiment, and/or 8.5% or less in another embodiment.

System 10-E can be configured, such as by configuration of on-body device 4601-E and/or the software application of receiver unit 4641-E, to apply a factory calibration to, in part, calculate an analyte value for display to the user, as described with respect to system 10-A.

System 10-E and receiver unit 4641-E can be further configured according to any of the embodiments described herein.

### System 10-F

An example embodiment of a system 10-F is shown in the landscape view of FIG. 46F. Here, system 10-F includes on-body device 4601-F, inserter 4621-F, receiver unit 4641-F, and/or overpatch 4661-F. On-body device 4601-F can be configured in accordance with any of the embodiments of on-body device 100 described herein. On-body device 4601-A is shown in both top down and side views. Here, on-body device 4601-F includes a housing 4602-F having a first part 4603-F and a second part 4604-F attached together. In this embodiment, the peripheral boundary 4606-F of housing 4602-F is configured like the embodiment described with respect to FIG. 1N-12, in a stadium or obround shape with semi-circular end portions connected by straight and parallel portions. This shape may be interrupted by features. For example, housing 4602-F may include at least one recess 4607-F adapted to interface with a projection of inserter 4621-F, e.g., such as an arm, a rim or other snap projection, that is configured to releasably hold on-body device 4601-F, for example, as described with respect to any of the embodiments of FIGS. 1U-16 through 1U-22. Here, housing 4602-F includes four recesses, 4607-F-1, 4607-F-2, 4608-F-1, and 4608-F-2. Housing 4602-F may include a recess (on the underside of part 4603-F) configured to receive a sensor connector 4610-F as described with respect to FIGS. 1K-1 and 1K-2. Housing 4602-D includes a sharp aperture 4615-D. Housing 4602-F may be water-resistant. For example, housing 4602-F may withstand immersion in about one (1) meter of water for 30 minutes.

Sensor electronics 122 (not shown), such as described with respect to FIG. 1R-1, may be retained within housing 4602-F. Sensor electronics 122 may include processing electronics present on a semiconductor chip. Sensor electronics 122 may further include wireless communications electronics present on the semiconductor chip and configured to communicate according to a Bluetooth Low Energy protocol. Sensor electronics 122 may also include a coin cell battery as a power source 320 and a magnetic switch to activate some or all of the sensor electronics in response to a magnetic field. Some of sensor electronics 122 may be coupled to a printed circuit board. Sensor electronics 122 may include an antenna configured to communicate according to the Bluetooth Low Energy protocol.

On-body device 4601-F includes an adhesive patch 4605-F attached to the housing 4602-F and configured to attach the housing to skin of the user. Adhesive patch 4605-F may protrude beyond a peripheral boundary 4606-F of housing 4602-F as shown. Adhesive patch 4605-F can be configured in accordance with any of the embodiments of adhesive patches described herein, including those adhesive patch embodiments with one or more removable patch portions (as described herein in the section titled ‘REMOVABLE PATCH PORTIONS’), one or more non-isotropic layers (as described herein in the section titled ‘NON-ISOTROPIC PATCHES’) and/or variations (e.g., openings, polymeric layers, etc.) thereof, layered material constructions (e.g., such as described with respect to FIG. 45), or any combination thereof. Adhesive patch 4605-F can have a circular outer periphery as shown here, or a periphery with a shape corresponding to the outer periphery of housing 4602-F, or otherwise.

On-body device 4601-F may include a sensor 161A, such as described with respect to the embodiments of FIGS. 1O-1, 1O-2, 1O-3, 1O-4, 1Q-1, and/or 1Q-2. Sensor 161A may be coupled to the sensor connector 4610-F. Sensor 161A may have a flexible substrate and may include a first portion retained within the housing and including three contacts configured to be in electrical contact with the sensor electronics. Sensor 161A may include a second portion configured to be positioned in the body of the user for monitoring a level of an analyte of the user. The second portion may have a length and a width arranged in a plane and may have a thickness perpendicular to the plane as depicted in the end on view of FIG. 1O-5. The thickness may be smaller than the width. The second portion may include three electrodes connected to connections that are in turn connected to the three contacts of the first portion. Sensor 161A may have a fold such as described with respect to FIG. 1Q-2. Alternatively, sensor 161A may not have a fold, such as described with respect to FIG. 1Q-1.

On-body device 4601-F can be further configured according to any of the embodiments described herein, including with one or more vent passageways (as described herein in the section titled ‘VENT PASSAGEWAY’), one or more vent seals (as described herein in the section titled ‘VENT SEAL’), one or more moisture egress channels (as described herein in the section titled ‘ADDITIONAL MOISTURE EGRESS FEATURES—Channels’), one or more surface textures (as described herein in the section titled ‘ADDITIONAL MOISTURE EGRESS FEATURES—Surface Texture’), one or more reinforcement devices (as described herein in the section titled ‘REINFORCEMENT DEVICES’), one or more anti-stick layers, or any combination thereof.

System 10-F can include an overpatch 4661-F configured to be applied over on-body device 4601-F and/or adhesive patch adhesive patch 4605-F. Overpatch 4661-F can be configured in accordance with any of the embodiments of adhesive overpatches described herein, including those adhesive patch embodiments with one or more removable patch portions, one or more non-isotropic layers and/or variations (e.g., openings, polymeric layers, etc.) thereof, layered material constructions (e.g., such as described with respect to FIG. 45), one or more support structures, one or more removable liners, or any combination thereof. In this embodiment, overpatch 4661-F has an inner periphery 4642-F about an opening that corresponds to the shape of the periphery of housing 4602-F of on-body device 4601-F. Overpatch 4661-F also may include a support structure 4664-F that has a body relatively more rigid than the flexible patch material and is in a shape corresponding to periphery 4606-F of housing 4602-F (in this embodiment a stadium or obround shape). Overpatch 4661-F may have an outer periphery 4663-F having a shape that does or does not correspond to the shape of the periphery 4606-F of housing 4602-F. Other shapes (e.g., circular, oval, asymmetric, polygonal or a combination thereof) for inner periphery 4662-F and outer periphery 4663-F may also be used.

On-body device 4601-F may be provided to an end user in inserter 4621-F. Sensor connector 4610-F may be provided to the end user in a container or tray 4626-F that is discrete from inserter 4621-F. Sensor connector 4610-F may include the sharp, sensor 161A, and a connector piece holding both, and may be maintained in a sterile state within container 4626-F. Inserter 4621-F is mated with the sensor connector within container 4626-F to connect the sensor connector with on-body device 100 similar to the embodiment described with respect to FIGS. 1U-7 and 1U-8.

Inserter 4621-F may include a sharp, a first spring configured to advance the sharp, and a second spring configured to retract the sharp into housing 4624-F. Inserter 4621-F may include a housing 4624-F having an actuator 4625-F configured to initiate advancement of the sharp and on-body device 4601-F when mated with the sensor connector. The actuator 4625-F may be configured as a push button. A removable locking element 4628-F may be coupled to the inserter body and/or housing 4624-F in proximity to actuator 4625-F, the presence of which prevents actuation, and removal of which allows actuation of actuator 4625-F. Here, locking element 4628-F is configured as an elastic clip. The first spring may be a component of an insertion mechanism and the second spring may be a component of a retraction mechanism as described herein. The interior space of inserter 4621-F may be sealed by a removable cap 4622-F coupled with housing 4624-F. Inserter 4621-F may be as described with respect to FIGS. 1T-4, 1T-5, 1T-6, 1U-4, 1U-5, and/or 1U-6 and configured to automatically advance a sharp and move on-body device 4601-F within inserter 4621-F, and then to automatically retract the sharp.

The sharp may be configured as described with respect to any of FIGS. 1U-23 through 1U-30. The sharp may include a distal tip portion with a V-shaped portion having an angle (e.g., angle 661) of 20 to 60 degrees and, in some embodiments, the angle can be between 20 and 30 degrees. The sharp may include a proximal shaft portion having a first sidewall, a floor, and a second sidewall with a U-shape or a C-shape cross-sectional profile. The sharp may include a transition portion between the distal tip portion and the proximal shaft portion where the first sidewall and the second sidewall each have a height from the floor that is sloped at an angle (e.g., angle 669) of 7 to 35 degrees and, in some embodiments, the angle is between 8 and 18 degrees. On-body device 4601-F may include the sharp aperture 4615-F for passage of the sharp through housing 4602-F.

Inserter 4621-F can be further configured according to any of the embodiments described herein.

Receiver unit 4641-F may be a dedicated receiver unit built for the on-body device manufacturer for the purpose of communicating with on-body device 4601-F. Receiver unit 4641-F may alternatively be a smart phone or a smart watch. Receiver unit 4641-F may include processing electronics communicatively coupled with memory as described with respect to FIG. 1R-13. Receiver unit 4641-F may include a software application 4644-F stored thereon and for execution by the receiver unit (e.g., by the processing electronics). Receiver unit 4641-F may include a display 4645-F for outputting analyte values and other information to a user. Receiver unit 4641-F may be configured to receive analyte data associated with the analyte level of the user from on-body device 4601-F and display information corresponding to the analyte data to the user. Display 4645-F may be configured as a touchscreen permitting user input. Other tangible user input interfaces 4646-F can be included.

The software application may be configured to generate a present analyte value of the on-body device wearer (the sensor recipient), based on a measurement by the sensor, for output to the user. System 10-F can be configured, such as by configuration of on-body device 4601-F and/or the software application of receiver unit 4641-F, so that the analyte value of the wearer is not displayed before expiration of a warm-up period of the sensor. The warm-up period is a period of time after activation of the sensor where the sensor may adjust to the conditions of the human body and obtain a normal or optimal operating condition for obtaining accurate measurements. The warm up period may be 30 minutes or less, may be one hour, may be two hours, or may be longer depending on the design and construction of the sensor.

System 10-F can be configured, such as by configuration of on-body device 4601-F and/or the software application of receiver unit 4641-F, so that a mean average relative difference (MARD) of the analyte values displayed to the user are 9.5% or less in one embodiment, 9.0% or less in another embodiment, and/or 8.5% or less in another embodiment.

System 10-F can be configured, such as by configuration of on-body device 4601-F and/or the software application of receiver unit 4641-F, to apply a factory calibration to, in part, calculate an analyte value for display to the user, as described with respect to system 10-A.

System 10-F and receiver unit 4641-F can be further configured according to any of the embodiments described herein.

### System 10-G

An example embodiment of a system 10-G is shown in the landscape view of FIG. 46G. Here, system 10-G includes on-body device 4601-G, inserter 4621-G, receiver unit 4641-G, and/or overpatch 4661-G. On-body device 4601-G can be configured in accordance with any of the embodiments of on-body device 100 described herein. On-body device 4601-A is shown in both top down and side views. Here, on-body device 4601-G includes a housing 4602-G having a first part 4603-G and a second part 4604-G attached together. In this embodiment, the peripheral boundary 4606-G of housing 4602-G is configured like the embodiment described with respect to FIG. 1N-12, in a stadium or obround shape with semi-circular end portions connected by straight and parallel portions. This shape may be interrupted by features. For example, housing 4602-G may include at least one recess 4607-G adapted to interface with a projection of inserter 4621-G, e.g., such as a rim or other snap projection, that is configured to releasably hold on-body device 4601-G, for example, as described with respect to any of the embodiments of FIGS. 1U-16 through 1U-22. Housing 4602-G includes a sharp aperture 4615-G. Housing 4602-G may be water-resistant. For example, housing 4602-G may withstand immersion in about one (1) meter of water for 30 minutes.

Sensor electronics 122 (not shown), such as described with respect to FIG. 1R-1, may be retained within housing 4602-G. Sensor electronics 122 may include processing electronics present on a semiconductor chip. Sensor electronics 122 may further include wireless communications electronics present on the semiconductor chip and configured to communicate according to a Bluetooth Low Energy protocol. The wireless communications electronics may also be configured to communicate according to a Near Field Communication (NFC) protocol, and that portion of the wireless communication electronics capable of NFC communication can be on the semiconductor chip or located elsewhere in on-body device 4601-G. Sensor electronics 122 may also include a coin cell battery as a power source 320 and a magnetic switch to activate some or all of the sensor electronics in response to a magnetic field. Some of sensor electronics 122 may be coupled to a printed circuit board. Sensor electronics 122 may include a first antenna configured to communicate according to the Bluetooth Low Energy protocol and a second antenna configured to communicate according to the NFC protocol. The first antenna can be printed on the printed circuit board or can be a discrete component coupled with the printed circuit board. In either embodiment, the second antenna can be printed on the printed circuit board or can be a discrete component coupled with the printed circuit board. The coin cell battery 320 may be electrically connected to the printed circuit board and held adjacent to the printed circuit board such that the coin cell battery and the printed circuit board are both in a same plane as described with respect to FIGS. 1R-7, 1R-8, and/or 1R-9.

On-body device 4601-G includes an adhesive patch 4605-G attached to the housing 4602-G and configured to attach the housing to skin of the user. Adhesive patch 4605-G may protrude beyond a peripheral boundary 4606-G of housing 4602-G as shown. Adhesive patch 4605-G can be configured in accordance with any of the embodiments of adhesive patches described herein, including those adhesive patch embodiments with one or more removable patch portions (as described herein in the section titled ‘REMOVABLE PATCH PORTIONS’), one or more non-isotropic layers (as described herein in the section titled ‘NON-ISOTROPIC PATCHES’) and/or variations (e.g., openings, polymeric layers, etc.) thereof, layered material constructions (e.g., such as described with respect to FIG. 45), or any combination thereof. Adhesive patch 4605-G can have a shape corresponding to the outer periphery of housing 4602-A as shown here, a circular outer periphery, or a periphery with a different shape.

On-body device 4601-G may include a sensor 161A, such as described with respect to the embodiments of FIGS. 1O-1, 1O-2, 1O-3, 1O-4, 1Q-1, and/or 1Q-2. Sensor 161A may have a flexible substrate and may include a first portion retained within the housing and including three contacts configured to be in electrical contact with the sensor electronics. Sensor 161A may include a second portion configured to be positioned in the body of the user for monitoring a level of an analyte of the user. The second portion may have a length and a width arranged in a plane and may have a thickness perpendicular to the plane as depicted in the end on view of FIG. 1O-5. The thickness may be smaller than the width. The second portion may include three electrodes connected to connections that are in turn connected to the three contacts of the first portion. Sensor 161A may have a fold such as described with respect to FIG. 1Q-2. Alternatively, sensor 161A may not have a fold, such as described with respect to FIG. 1Q-1.

On-body device 4601-G can be further configured according to any of the embodiments described herein, including with one or more vent passageways (as described herein in the section titled ‘VENT PASSAGEWAY’), one or more vent seals (as described herein in the section titled ‘VENT SEAL’), one or more moisture egress channels (as described herein in the section titled ‘ADDITIONAL MOISTURE EGRESS FEATURES—Channels’), one or more surface textures (as described herein in the section titled ‘ADDITIONAL MOISTURE EGRESS FEATURES—Surface Texture’), one or more reinforcement devices (as described herein in the section titled ‘REINFORCEMENT DEVICES’), one or more anti-stick layers, or any combination thereof.

System 10-G can include an overpatch 4661-G configured to be applied over on-body device 4601-G and/or adhesive patch adhesive patch 4605-G. Overpatch 4661-G can be configured in accordance with any of the embodiments of adhesive overpatches described herein, including those adhesive patch embodiments with one or more removable patch portions, one or more non-isotropic layers and/or variations (e.g., openings, polymeric layers, etc.) thereof, layered material constructions (e.g., such as described with respect to FIG. 45), one or more support structures, one or more removable liners, or any combination thereof. In this embodiment, overpatch 4661-G has an inner periphery 4642-G about an opening that corresponds to the shape of the periphery of housing 4602-G of on-body device 4601-G. Overpatch 4661-G also may include a support structure 4664-G that has a body relatively more rigid than the flexible patch material and is in a shape corresponding to periphery 4606-G of housing 4602-G (in this embodiment a stadium or obround shape). Overpatch 4661-G may have an outer periphery 4663-G having a shape that does or does not correspond to the shape of the periphery 4606-G of housing 4602-G. Other shapes (e.g., circular, oval, asymmetric, polygonal or a combination thereof) for inner periphery 4662-G and outer periphery 4663-G may also be used.

On-body device 4601-G may be housed within an interior space of inserter 4621-G with the interior space being sealed by removable cap 4622-G coupled with housing 4624-G of inserter 4621-G. Cap 4622-G can be removed by twisting to release from the housing 4624-G. Cap 4622-G may include a sheath that covers the sensor and sharp and maintains them in a sterile environment (such as described with respect to FIGS. 1U-14 and 1U-15). Removal of cap 4622-G may remove the sheath and expose the sensor and sharp to the ambient environment.

On-body device 4601-G may be pre-loaded and housed within an interior space of inserter 4621-G. Inserter 4621-G may include a housing 4624-G having an actuator 4625-G configured to initiate advancement of the sharp. The actuator 4625-G may be configured as a push button. Inserter 4621-G may include a sharp, a first spring configured to advance the sharp, and a second spring configured to retract the sharp into housing 4624-G. The first spring may be a component of an insertion mechanism and the second spring may be a component of a retraction mechanism as described herein. The interior space of inserter 4621-G may be sealed by a removable cap 4622-G coupled with housing 4624-G. Inserter 4621-G may be as described with respect to FIGS. 1T-4, 1T-5, and 1T-6 and configured to automatically advance a sharp and move on-body device 4601-G within inserter 4621-G, and then to automatically retract the sharp. A magnet may be present within inserter 4621-G in a fixed position such that the on-body device 100 is activated as a device support carries the on-body device away from the magnet (as described herein in the section titled ‘INSERTERS AND APPLICATORS’).

The sharp may be configured as described with respect to any of FIGS. 1U-23 through 1U-30. The sharp may include a distal tip portion with a V-shaped portion having an angle (e.g., angle 661) of 20 to 60 degrees and, in some embodiments, the angle can be between 27 and 37 degrees. The sharp may include a proximal shaft portion having a first sidewall, a floor, and a second sidewall with a U-shape or a C-shape cross-sectional profile. The sharp may include a transition portion between the distal tip portion and the proximal shaft portion where the first sidewall and the second sidewall each have a height from the floor that is sloped at an angle (e.g., angle 669) of 7 to 35 degrees and, in some embodiments, the angle is between 7 and 17 degrees. Sharp aperture 4615-G permits passage of the sharp through housing 4602-G.

Inserter 4621-G can be further configured according to any of the embodiments described herein.

Receiver unit 4641-G may be a dedicated receiver unit built for the on-body device manufacturer for the purpose of communicating with on-body device 4601-G. Receiver unit 4641-G may alternatively be a smart phone or a smart watch. Receiver unit 4641-G may include processing electronics communicatively coupled with memory as described with respect to FIG. 1R-13. Receiver unit 4641-G may include a software application stored thereon and for execution by the receiver unit (e.g., by the processing electronics). Receiver unit 4641-G may include a display 4645-G for outputting analyte values and other information to a user. Receiver unit 4641-G may be configured to receive analyte data associated with the analyte level of the user from on-body device 4601-G and display information corresponding to the analyte data to the user. Display 4645-G may be configured as a touchscreen permitting user input. Other tangible user input interfaces 4646-G can be included.

The software application may be configured to generate a present analyte value of the on-body device wearer (the sensor recipient), based on a measurement by the sensor, for output to the user. System 10-G can be configured, such as by configuration of on-body device 4601-G and/or the software application of receiver unit 4641-G, so that the analyte value of the wearer is not displayed before expiration of a warm-up period of the sensor. The warm-up period is a period of time after activation of the sensor where the sensor may adjust to the conditions of the human body and obtain a normal or optimal operating condition for obtaining accurate measurements. The warm up period may be 30 minutes or less, may be one hour, may be two hours, or may be longer depending on the design and construction of the sensor.

System 10-G can be configured, such as by configuration of on-body device 4601-G and/or the software application of receiver unit 4641-G, so that a mean average relative difference (MARD) of the analyte values displayed to the user are 9.5% or less in one embodiment, 9.0% or less in another embodiment, and/or 8.5% or less in another embodiment.

System 10-G can be configured, such as by configuration of on-body device 4601-G and/or the software application of receiver unit 4641-G, to apply a factory calibration to, in part, calculate an analyte value for display to the user, as described with respect to system 10-A.

System 10-G and receiver unit 4641-G can be further configured according to any of the embodiments described herein.

The embodiments described herein are reiterated and/or supplemented in the following paragraphs which do so without explicit reference to the figures. These paragraphs reference clauses which provide explicit written support for claims of like character. Any term or expression used to describe a feature in the clauses shall be understood to refer to the same feature throughout. The features of an on-body device, as set forth in the clauses, may be combined in any appropriate combination.

Clause 1. An on-body device for monitoring an analyte level of a user, comprising: a housing, comprising: a first surface comprising an aperture and a vent inlet; and a second surface comprising a vent outlet; wherein the vent inlet is fluidly connected to the vent outlet through the housing to allow passage of water vapor from the first surface to the second surface; an adhesive patch attached to the housing and configured to attach the housing to skin of the user; sensor electronics, wherein the housing is configured to retain the sensor electronics; and a sensor, comprising: a first portion configured to be in electrical contact with the sensor electronics; and a second portion configured to be positioned in the body of the user for monitoring a level of an analyte of the user; wherein the sensor is configured to extend through the aperture for positioning the second portion of the sensor in the body of the user.

Clause 2. The on-body device of clause 1, wherein the first surface is a base surface of the housing.

Clause 3. The on-body device of clause 1 or 2, wherein the second surface is a top surface of the housing.

Clause 4. The on-body device of clause 1 or 2, wherein the second surface is a side surface of the housing.

Clause 5. The on-body device of any preceding clause, wherein the vent inlet is spaced away from a periphery of the first surface.

Clause 6. The on-body device of any preceding clause, wherein the vent outlet is aligned with the vent inlet along an axis perpendicular to the first surface.

Clause 7. The on-body device of any preceding clause, wherein the vent outlet is smaller than the vent inlet.

Clause 8. The on-body device of any preceding clause, wherein the vent outlet comprises a width of less than 1 mm.

Clause 9. The on-body device of any preceding clause, wherein the housing comprises a vent passageway between the vent inlet and the vent outlet.

Clause 10. The on-body device of clause 9, wherein the vent passageway has a longitudinal axis that is normal to at least a portion of the first surface at the vent inlet.

Clause 11. The on-body device of clause 9, wherein the vent passageway has a longitudinal axis that is angled with respect to at least a portion of the first surface at the vent inlet.

Clause 12. The on-body device of clause 11, wherein the longitudinal axis of the vent passageway is angled from the vent inlet to the vent outlet in a direction towards a periphery of the housing.

Clause 13. The on-body device of clause 11, wherein the longitudinal axis of the vent passageway is angled from the vent inlet to the vent outlet in a direction away from a periphery of the housing.

Clause 14. The on-body device of clause 9, wherein the vent inlet is wider than the vent outlet, and the vent passageway comprises a tapered width portion.

Clause 15. The on-body device of clause 9, wherein the vent outlet is wider than the vent inlet, and the vent passageway comprises a tapered width portion.

Clause 16. The on-body device of clause 9, wherein the vent inlet is wider than the vent outlet, and the vent passageway comprises a step transition in width from the vent inlet to the vent outlet.

Clause 17. The on-body device of clause 9, wherein the vent passageway has a first portion that extends along a first longitudinal axis, and a second portion that extends along a second longitudinal axis, wherein the first longitudinal axis is transverse to the second longitudinal axis.

Clause 18. The on-body device of clause 17, wherein the first longitudinal axis is perpendicular to the second longitudinal axis.

Clause 19. The on-body device of clause 9, wherein the vent passageway comprises a portion that extends along a curved longitudinal axis.

Clause 20. The on-body device of clause 9, wherein the entire vent passageway extends along a curved longitudinal axis.

Clause 21. The on-body device of any one of clauses 17-20, wherein the vent inlet is relatively farther from the periphery than the vent outlet.

Clause 22. The on-body device of any one of clauses 17-20, wherein the vent inlet is relatively closer to the periphery than the vent outlet.

Clause 23. The on-body device of any one of clauses 9-22, wherein the vent passageway extends through the sensor electronics.

Clause 24. The on-body device of clause 23, wherein the vent passageway extends through an aperture in a printed circuit board of the sensor electronics.

Clause 25. The on-body device of any one of clauses 9 to 24, wherein the aperture in the first surface is connected to the vent passageway to form a sensor passageway through the housing between the aperture and the vent passageway, and wherein the vent outlet is configured to receive a sharp for positioning the second portion of the analyte sensor in the body of the user.

Clause 26. The on-body device of any of clauses 9 to 24, wherein the second surface comprises a second aperture, wherein the aperture in the first surface is connected to the second aperture in the second surface to form a sensor passageway through the housing, wherein the second aperture is configured to receive a sharp for positioning the second portion of the analyte sensor in the body of the user, and wherein the vent passageway extends from the vent inlet to the vent outlet.

Clause 27. The on-body device of any one of clauses 1 to 26, wherein the housing comprises a vent compartment, wherein the vent inlet is fluidly connected to the vent compartment, and wherein the vent compartment is fluidly connected to the vent outlet.

Clause 28. The on-body device of clause 27, wherein the vent compartment is fluidly connected to at least one vent passageway.

Clause 29. The on-body device of any preceding clause, wherein the housing comprises at least one channel along the first surface.

Clause 30. The on-body device of clause 29, wherein the at least one channel comprises a first end connected to the vent inlet.

Clause 31. The on-body device of any preceding clause, wherein the first surface comprises a plurality of vent inlets.

Clause 32. The on-body device of any preceding clause, wherein the second surface comprises a plurality of vent outlets.

Clause 33. The on-body device of any preceding clause, wherein the on-body device comprises a plurality of vent passageways, a plurality of vent inlets, and a plurality of vent outlets.

Clause 34. The on body-unit of clause 33, wherein each vent passageway of the plurality of vent passageways extends from a vent inlet of the plurality of vent inlets to a vent outlet of the plurality of vent outlets.

Clause 35. The on body-unit of clause 34, wherein no two vent passageways of the plurality of vent passageways share a vent inlet of the plurality of vent inlets nor a vent outlet of the plurality of vent outlets.

Clause 36. The on-body device of any one of clauses 33 to 35, wherein the on-body device comprises two vent passageways.

Clause 37. The on-body device of any one of clauses 33 to 35, wherein the on-body device comprises three vent passageways.

Clause 38. The on-body device of any one of clauses 33 to 35, wherein the on-body device comprises four vent passageways.

Clause 39. The on-body device of any one of clauses 33 to 35, wherein the on-body device comprises five vent passageways.

Clause 40. The on-body device of any one of clauses 33 to 35, wherein the on-body device comprises six vent passageways.

Clause 41. The on-body device of any one of clauses 33 to 35, wherein the on-body device comprises seven vent passageways.

Clause 42. The on-body device of any one of clauses 33 to 35, wherein the on-body device comprises eight vent passageways.

Clause 43. The on-body device of any one of clauses 33 to 35, wherein the on-body device comprises between two and ninety vent passageways.

Clause 44. The on-body device of any one of the preceding clauses, further comprising a moisture permeable seal.

Clause 45. The on-body device of clause 44, wherein the moisture permeable seal is at least partially within a first passageway between the first surface and the second surface, wherein the first passageway is the vent passageway.

Clause 46. The on-body device of clause 44, wherein the moisture permeable seal is at least partially within a first passageway between the first surface and the second surface, wherein the first passageway is the sensor passageway.

Clause 47. The on-body device of any one of clauses 45 and 46, wherein the moisture permeable seal is located entirely within the first passageway.

Clause 48. The on-body device of any one of clauses 45 to 47, wherein the first surface is a lower base surface of the housing and the second surface is above the first surface in a position relatively farther from a body of the user, wherein the first passageway extends between the lower base surface and the second surface.

Clause 49. The on-body device of clause 48, wherein the moisture permeable seal is located at an upper location, an intermediate location, or a lower location within the first passageway.

Clause 50. The on-body device of any one of clauses 44 to 49, wherein the moisture permeable seal is a first moisture permeable seal, the on-body device further comprising a second moisture permeable seal configured to allow passage of water vapor through the first passageway.

Clause 51. The on-body device of clause 50, wherein the first moisture permeable seal is located at an upper location within the passageway and the second moisture permeable seal is located at a lower location within the passageway.

Clause 52. The on-body device of any one of clauses 44 to 49, wherein the moisture permeable seal has a thickness that is 50% or less of an entire length of the first passageway.

Clause 53. The on-body device of any one of clauses 44 to 49, wherein the moisture permeable seal has a thickness that is 1%-35% of an entire length of the first passageway.

Clause 54. The on-body device of any one of clauses 44 to 49, wherein the moisture permeable seal has a thickness that is 1%-25% of an entire length of the passageway.

Clause 55. The on-body device of any one of clauses 44 to 49, wherein the housing comprises a recess in a wall of the passageway, and wherein at least a portion of the moisture permeable seal is in the recess.

Clause 56. The on-body device of clause 55, wherein the recess is annular.

Clause 57. The on-body device of any one of clauses 44 to 54, wherein the housing comprises a projection from a wall of the first passageway into an interior of the first passageway, and wherein at least a portion of the moisture permeable seal abuts the projection.

Clause 58. The on-body device of clause 57, wherein the projection is annular.

Clause 59. The on-body device of any one of clauses 44 to 58, wherein the moisture permeable seal comprises a flange and a columnar portion.

Clause 60. The on-body device of clause 59, wherein the flange and columnar portion are located within the passageway, such that an end surface of the columnar portion is flush with a surface of the housing.

Clause 61. The on-body device of clause 59, wherein the moisture permeable seal further comprises a head portion configured to reside outside of the passageway and abut a surface of the housing.

Clause 62. The on-body device of clause 44, wherein the moisture permeable seal is configured as a liner.

Clause 63. The on-body device of clause 62, wherein the liner comprises adhesive.

Clause 64. The on-body device of clause 63, wherein the adhesive is configured to attach the liner to a second surface of the housing such that the liner covers the first passageway.

Clause 65. The on-body device of clause 64, wherein the second surface of the housing is an upper surface configured to face away from a body of a wearer of the on-body device.

Clause 66. The on-body device of clause 44, wherein the first surface is a lower base surface and the second surface is above the first surface in a position relatively farther from a body of the user, wherein the first passageway extends between the lower base surface and the second surface, and wherein the moisture permeable seal is configured as a cap for the opening in the second surface.

Clause 67. The on-body device of clause 66, wherein the cap comprises a roof member and a sidewall.

Clause 68. The on-body device of clause 67, wherein the sidewall comprises a first retention feature.

Clause 69. The on-body device of clause 68, wherein the first retention feature is configured to interlock with a second retention feature in a receiving space within the housing.

Clause 70. The on-body device of any one of clauses 66 to 69, wherein the cap is configured to be manually attached to the housing by a user.

Clause 71. The on-body device of any one of clauses 66 to 70, wherein the cap comprises an inner passageway for passage of a sharp therethrough.

Clause 72. The on-body device of any one of clauses 44 to 71, wherein the first passageway extends between the aperture in the first surface and the vent outlet.

Clause 73. The on-body device of any one of clauses 44 to 71, wherein the first passageway extends between the vent inlet in the first surface and the vent outlet.

Clause 74. The on-body device of one of clauses 44 to 71, wherein the first passageway is a vent passageway that extends between the vent inlet and the vent outlet, the housing further comprising a sensor passageway that extends between the aperture in the first surface and an aperture in the second surface, wherein the moisture permeable seal is a first moisture permeable seal in the vent passageway, wherein the housing comprises a second moisture permeable seal in the sensor passageway, the second moisture permeable seal configured to allow the passage of water vapor through the sensor passageway.

Clause 75. The on-body device of clause 44, wherein the moisture permeable seal comprises an adhesive and is configured to adhere to the second surface of the housing, wherein the second surface is opposite the first surface of the housing.

Clause 76. The on-body device of clause 75, wherein the moisture permeable seal is configured to cover the vent outlet.

Clause 77. The on-body device of clause 44, wherein the housing comprises a plurality of passageways, including at least one vent passageway and a sensor passageway, and wherein the moisture permeable seal is configured to cover each of the plurality of passageways.

Clause 78. The on-body device of any one of clauses 75 to 77, wherein the moisture permeable seal is configured as a sticker.

Clause 79. The on-body device of any one of clauses 44 to 78, wherein the moisture permeable seal comprises an inner passageway configured to permit passage of a sharp therethrough.

Clause 80. The on-body device of clause 79, wherein the moisture permeable seal comprises an elastic material biased to self-close the inner passageway.

Clause 81. The on-body device of clause 44, wherein the moisture permeable seal comprises a columnar portion configured to reside within the first passageway and a head portion configured to abut the first surface of the housing.

Clause 82. The on-body device of clause 81, wherein the adhesive patch holds the moisture permeable seal against the first surface.

Clause 83. The on-body device of any of clauses 9 to 82, wherein the moisture permeable seal comprises a moisture permeable material.

Clause 84. The on-body device of clause 83, wherein the moisture permeable seal comprises silicone.

Clause 85. The on-body device of clause 83, wherein the moisture permeable material comprises poly(methyl methacrylate).

Clause 86. The on-body device of clause 83, wherein the moisture permeable material comprises a moisture permeable fibrous material.

Clause 87. The on-body device of clause 83, wherein the moisture permeable material comprises polyurethane (PU).

Clause 88. The on-body device of clause 83, wherein the moisture permeable material comprises polytetrafluoroethylene (PTFE).

Clause 89. The on-body device of clause 83, wherein the moisture permeable material comprises polyethylene (PE).

Clause 90. The on-body device of clause 83, wherein the moisture permeable material comprises nylon (polyamide).

Clause 91. The on-body device of any one of clauses 44 to 90, wherein the moisture permeable seal is configured to allow passage of water vapor therethrough and block passage of liquid water through the passageway.

Clause 92. The on-body device of any one of clauses 44 to 91, wherein the moisture permeable seal is configured to allow accumulated moisture to exit a region between the on-body device housing and a body of the user by evaporation into a vapor and then migration through the moisture permeable seal into an ambient external environment away from the user's body.

Clause 93. The on-body device of any one of clauses 44 to 92, wherein the moisture permeable seal comprises two or more moisture permeable materials, or a moisture permeable material and a liquid permeable material.

Clause 94. The on-body device of any one of clauses 44 to 92, wherein the moisture permeable seal is fibrous.

Clause 95. The on-body device of any one of clauses 44 to 94, wherein the moisture permeable seal comprises perforations having a diameter between 10 and 1000 microns.

Clause 96. The on-body device of any one of clauses 44 to 94, wherein the moisture permeable seal comprises perforations having a diameter between 100 and 700 microns.

Clause 97. The on-body device of any one of clauses 44 to 94, wherein the moisture permeable seal comprises perforations having a diameter between 200 and 600 microns.

Clause 98. The on-body device of any one of clauses 44 to 94, wherein the moisture permeable seal comprises perforations having a diameter between 300 and 500 microns.

Clause 99. The on-body device of any one of clauses 97 to 98, wherein the moisture permeable seal comprises a hydrophilic material or material bearing a hydrophilic coating.

Clause 100. The on-body device of any one of clauses 44 to 99, wherein the on-body device has a Moisture Vapor Transmission Rate (MVTR) of 35 grams per square meter per hour (g/m2/hr) or more.

Clause 101. The on-body device of any one of clauses 1 to 100, further comprising a solid foam.

Clause 102. The on-body device of clause 101, wherein the solid foam is a foam seal.

Clause 103. The on-body device of any one of clauses 101 to 102, wherein the solid foam is an open-celled foam, a closed-celled foam, or a combination thereof.

Clause 104. The on-body device of any one of clauses 101 to 103, wherein the solid foam is configured to impede significant or total liquid water ingress while allowing moisture vapor and limited amounts of liquid to pass through.

Clause 105. The on-body device of any one of clauses 101 to 104, wherein the solid foam comprises a reticulated polyurethane foam.

Clause 106. The on-body device of any one of clauses 101 to 104, wherein the solid foam comprises a hydrophobic treated open-cell foam.

Clause 107. The on-body device of any one of clauses 101 to 104, wherein the solid foam comprises a silicone open-cell foam.

Clause 108. The on-body device of any one of clauses 101 to 107, wherein the solid foam is located in a passageway of the housing between the first surface and the second surface.

Clause 109. The on-body device of clause 108, wherein the solid foam is located in the sensor passageway or the vent passageway.

Clause 110. The on-body device of clause 109, wherein the solid foam is in contact with the adhesive patch

Clause 111. The on-body device of any one of clauses 109 to 110, wherein the solid foam fills an entire length of the passageway.

Clause 112. The on-body device of any one of clauses 109 to 110, wherein the solid foam fills only a portion of the passageway.

Clause 113. The on-body device of clause 112, wherein the portion of the passageway has a length that is 25-35% of the length of the passageway.

Clause 114. The on-body device of clause 112, wherein the portion of the passageway has a length that is 35-50% of the length of the passageway.

Clause 115. The on-body device of clause 112, wherein the portion of the passageway has a length that is 35-75% of the length of the passageway.

Clause 116. The on-body device of clause 112, wherein the portion of the passageway has a length that is 5-15% of the length of the passageway.

Clause 117. The on-body device of clause 112, wherein the portion of the passageway has a length that is 15-25% of the length of the passageway.

Clause 118. The on-body device of clause 112, wherein the solid foam is located in an upper, intermediate, or lower location in the passageway.

Clause 119. The on-body device of any one of clauses 109 to 118, wherein the solid foam is in the sensor passageway and comprises a channel for the sensor to extend therethrough.

Clause 120. The on-body device of any one of clauses 44 to 119, wherein the solid foam is in contact with a moisture permeable material.

Clause 121. The on-body device of any one of clauses 44 to 100, further comprising a solid foam seal in a same passageway as the moisture permeable seal.

Clause 122. The on-body device of any one of clauses 44 to 100, wherein the moisture permeable seal is located at the vent outlet.

Clause 123. The on-body device of any one of clauses 44 to 100, wherein the moisture permeable seal is located at the vent inlet.

Clause 124. The on-body device of any one of clauses 44 to 100, wherein the moisture permeable seal is a first moisture permeable seal, the housing comprising a second moisture permeable seal in the same passageway as the first moisture permeable seal, with the solid foam located therebetween.

Clause 125. The on-body device of any one of clauses 44 to 100, wherein the solid foam is a first solid foam, the housing comprising a second solid foam in the same passageway as the first solid foam, with the moisture permeable seal located therebetween.

Clause 126. The on-body device of any one of clauses 1 to 125, further comprising a fibrous seal configured to transport liquid by capillary action.

Clause 127. The on-body device of clause 126, wherein the adhesive patch is different from the fibrous seal.

Clause 128. The on-body device of any one of clauses 1 to 127, wherein the analyte is glucose.

Clause 129. The on-body device of any one of clauses 1 to 127, wherein the analyte is one or more ketones.

Clause 130. The on-body device of any one of clauses 1 to 127, wherein the analyte is lactate.

Clause 131. The on-body device of any one of clauses 1 to 127, wherein the analyte is oxygen.

Clause 132. The on-body device of any one of clauses 1 to 127, wherein the analyte is ammonium.

Clause 133. The on-body device of any one of clauses 1 to 127, wherein the analyte is asparagine.

Clause 134. The on-body device of any one of clauses 1 to 127, wherein the analyte is aspartate.

Clause 135. The on-body device of any one of clauses 1 to 127, wherein the analyte is cholesterol.

Clause 136. The on-body device of any one of clauses 1 to 127, wherein the analyte is choline.

Clause 137. The on-body device of any one of clauses 1 to 127, wherein the analyte is creatinine.

Clause 138. The on-body device of any one of clauses 1 to 127, wherein the analyte is a diaphorase inhibitor.

Clause 139. The on-body device of any one of clauses 1 to 127, wherein the analyte is ethanol.

Clause 140. The on-body device of any one of clauses 1 to 127, wherein the analyte is glutamate.

Clause 141. The on-body device of any one of clauses 1 to 127, wherein the analyte is glycerol.

Clause 142. The on-body device of any one of clauses 1 to 127, wherein the analyte is lactose.

Clause 143. The on-body device of any one of clauses 1 to 127, wherein the analyte is phosphate.

Clause 144. The on-body device of any one of clauses 1 to 127, wherein the analyte is potassium.

Clause 145. The on-body device of any one of clauses 1 to 127, wherein the analyte is pyruvate.

Clause 146. The on-body device of any one of clauses 1 to 127, wherein the analyte is uric acid.

Clause 147. The on-body device of any one of clauses 1 to 127, wherein the analyte is a first analyte, and the sensor is further configured to measure a level of a second analyte of the user.

Clause 148. The on-body device of clause 147, wherein the first analyte is glucose and the second analyte is a ketone.

Clause 149. The on-body device of clause 147, wherein the first analyte is glucose and the second analyte is lactate.

Clause 150. The on-body device of clause 147, wherein the first analyte is lactate and the second analyte is a ketone.

Clause 151. The on-body device of clause 147, wherein the first and second analytes are selected from the list consisting of: ammonium, asparagine, aspartate, cholesterol, choline, creatinine, diaphorase inhibitors, ethanol, glucose, glutamate, glycerol, one or more ketones, lactate, lactose, oxygen, phosphate, potassium, pyruvate, and uric acid.

Clause 152. The on-body device of any one of clauses 1 to 151, wherein the on-body device has a designed wear duration of 15 to 30 days.

Clause 153. The on-body device of any one of clauses 1 to 151, wherein the on-body device has a designed wear duration of 31 to 45 days.

Clause 154. The on-body device of any one of clauses 1 to 151, wherein the on-body device has a designed wear duration that is 45 days or greater.

Clause 155. A method of monitoring an analyte of a user, comprising: applying an on-body device to a body of the user; and monitoring a level of the analyte with the on-body device, wherein the on-body device is configured in accordance with any one of clauses 1 to 154.

Clause 156. The method of clause 155, wherein monitoring the level of the analyte with the on-body device comprises monitoring the level of the analyte with the on-body device for a wear duration without removing the on-body device from the body of the user.

Clause 157. The method of clause 156, wherein the wear duration is 7 days or greater.

Clause 158. The method of clause 156, wherein the wear duration is 14 days or greater.

Clause 159. The method of clause 156, wherein the wear duration is 21 days or greater.

Clause 160. The method of clause 156, wherein the wear duration is 30 days or greater.

Clause 161. The method of clause 156, wherein the wear duration is 45 days or greater.

Clause 162. The method of clause 156, wherein the wear duration is between 7 days and 60 days, optionally wherein the wear duration is 28 days, optionally wherein the wear duration is 21 days, optionally wherein the wear duration is 15 days, optionally wherein the wear duration is 14 days.

Clause 163. The method of any one of clauses 155 to 162, wherein the analyte is a first analyte, the method further comprising monitoring a level of a second analyte with the on-body device.

Clause 164. The method of any one of clauses 155 to 162, wherein the analyte is a first analyte, the method further comprising monitoring a level of a second analyte with the on-body device for the wear duration.

Clause 165. The method of any one of clauses 163 to 164, further comprising transmitting, from the on-body device to a receiver unit, data indicative of the monitored level of the first analyte and the monitored level of the second analyte.

Clause 166. The method of any one of clauses 155 to 162, further comprising transmitting, from the on-body device to a receiver unit, data indicative of the monitored level of the analyte.

Clause 167. The method of clause 166, wherein the data indicative of the monitored level of the analyte is transmitted according to a Bluetooth or Bluetooth Low Energy (BLE) protocol.

Clause 168. The method of clause 166, wherein the data indicative of the monitored level of the analyte is transmitted according to a Near Field Communication (NFC) protocol.

Clause 169. The method of any one of clauses 166 to 168, wherein the receiver unit is a smart phone.

Clause 170. The method of any one of clauses 166 to 168, wherein the receiver unit is a dedicated receiver unit.

Clause 171. The method of any one of clauses 166 to 168, wherein the receiver unit is a smart watch.

Clause 172. The method of any one of clauses 155 to 162, further comprising transmitting, from the on-body device to a plurality of receiver units, data indicative of the monitored level of the analyte, wherein the plurality of receiver units are each in an authenticated state at the on-body device, and wherein the plurality of receiver units comprise a smart phone and a smart watch.

Clause 173. An insertion system, comprising: the on-body device for monitoring the level of the analyte of the user according to any one of clauses 1 to 154; and an inserter configured to position the on-body device on the skin of the user with the second portion of the analyte sensor positioned in the body of the user.

Clause 174. The system of clause 173, further comprising a receiver unit configured to receive data indicative of the analyte level from the on-body device.

Clause 175. The system of clause 174, wherein the receiver unit is configured to receive the data indicative of the monitored level of the analyte according to a Bluetooth or Bluetooth Low Energy (BLE) protocol.

Clause 176. The system of any one of clauses 174 and 175, wherein the receiver unit is configured to receive the data indicative of the monitored level of the analyte according to a Near Field Communication (NFC) protocol.

Clause 177. The system of clause 173, further comprising a plurality of receiver units configured to receive data indicative of the monitored level of the analyte from the on-body device, wherein the on-body device is configured to authenticate the plurality of receiver units, and wherein the plurality of receiver units comprise a smart phone and a smart watch.

Clause 178. An on-body device for monitoring an analyte level of a user, comprising: a housing, comprising: a first surface comprising a vent inlet; and a second surface comprising a vent outlet; wherein the vent inlet is fluidly connected to the vent outlet through the housing to allow passage of water vapor from the first surface to the second surface; an adhesive patch attached to the housing and configured to attach the housing to skin of the user; and electronics configured to receive a signal from a sensor configured to measure a level of an analyte of the user, wherein the signal comprises information indicative of a level of an analyte of the user, wherein the housing is configured to retain the electronics, and wherein the sensor is a wholly in vivo sensor or a wholly ex vivo sensor.

Clause 179. The on-body device of clause 178, wherein the electronics are not configured to operate with a partially in vivo sensor.

Clause 180. The on-body device of clause 178, wherein the sensor is a wholly in vivo sensor.

Clause 181. The on-body device of clause 180, wherein the sensor is configured to be implanted in its entirety in the body of the user such that no part of the sensor protrudes from the body of the user during analyte monitoring.

Clause 182. The on-body device of any one of clauses 180 to 181, wherein the electronics are configured to receive the signal wirelessly from the sensor while the sensor is implanted within the body of the user.

Clause 183. The on-body device of clause 182, wherein the electronics are configured to receive the signal according to a Bluetooth protocol.

Clause 184. The on-body device of clause 182, wherein the electronics are configured to receive the signal according to a Bluetooth Low Energy protocol.

Clause 185. The on-body device of clause 182, wherein the electronics are configured to receive the signal according to a Near Field Communication (NFC) protocol.

Clause 186. The on-body device of any one of clauses 178 to 179, wherein the sensor is a wholly ex vivo sensor, the on-body device further comprising the sensor.

Clause 187. The on-body device of clause 186, further comprising the sensor, wherein the sensor is housed in the housing and is configured to monitor the level of the analyte of the user while in the housing and without physical penetration of the body of the user.

Clause 188. The on-body device of any one of clauses 178 to 187, wherein the first surface is a base surface of the housing.

Clause 189. The on-body device of any one of clauses 178 to 188, wherein the second surface is a top surface of the housing.

Clause 190. The on-body device of any one of clauses 178 to 188, wherein the second surface is a side surface of the housing.

Clause 191. The on-body device of any one of clauses 178 to 190, wherein the vent inlet is spaced away from a periphery of the first surface.

Clause 192. The on-body device of any one of clauses 178 to 191, wherein the vent outlet is aligned with the vent inlet along an axis perpendicular to the first surface.

Clause 193. The on-body device of any one of clauses 178 to 192, wherein the vent outlet is smaller than the vent inlet.

Clause 194. The on-body device of any one of clauses 178 to 193, wherein the vent outlet comprises a width of less than 1 millimeter (mm).

Clause 195. The on-body device of any one of clauses 178 to 194, wherein the housing comprises a vent passageway between the vent inlet and the vent outlet.

Clause 196. The on-body device of clause 195, wherein the vent passageway has a longitudinal axis that is normal to at least a portion of the first surface at the vent inlet.

Clause 197. The on-body device of clause 195, wherein the vent passageway has a longitudinal axis that is angled with respect to at least a portion of the first surface at the vent inlet.

Clause 198. The on-body device of clause 197, wherein the longitudinal axis of the vent passageway is angled from the vent inlet to the vent outlet in a direction towards a periphery of the housing.

Clause 199. The on-body device of clause 197, wherein the longitudinal axis of the vent passageway is angled from the vent inlet to the vent outlet in a direction away from a periphery of the housing.

Clause 200. The on-body device of clause 195, wherein the vent inlet is wider than the vent outlet, and the vent passageway comprises a tapered width portion.

Clause 201. The on-body device of clause 195, wherein the vent outlet is wider than the vent inlet, and the vent passageway comprises a tapered width portion.

Clause 202. The on-body device of clause 195, wherein the vent inlet is wider than the vent outlet, and the vent passageway comprises a step transition in width from the vent inlet to the vent outlet.

Clause 203. The on-body device of clause 195, wherein the vent passageway has a first portion that extends along a first longitudinal axis, and a second portion that extends along a second longitudinal axis, wherein the first longitudinal axis is transverse to the second longitudinal axis.

Clause 204. The on-body device of clause 203, wherein the first longitudinal axis is perpendicular to the second longitudinal axis.

Clause 205. The on-body device of clause 195, wherein the vent passageway comprises a portion that extends along a curved longitudinal axis.

Clause 206. The on-body device of clause 195, wherein the entire vent passageway extends along a curved longitudinal axis.

Clause 207. The on-body device of any one of clauses 203 to 206, wherein the vent inlet is relatively farther from the periphery than the vent outlet.

Clause 208. The on-body device of any one of clauses 203 to 206, wherein the vent inlet is relatively closer to the periphery than the vent outlet.

Clause 209. The on-body device of any one of clauses 195 to 208, wherein the vent passageway extends through the electronics.

Clause 210. The on-body device of clause 209, wherein the vent passageway extends through an aperture in a printed circuit board of the electronics.

Clause 211. The on-body device of any one of clauses 178 to 210, wherein the housing comprises a vent compartment, wherein the vent inlet is fluidly connected to the vent compartment, and wherein the vent compartment is fluidly connected to the vent outlet.

Clause 212. The on-body device of clause 211, wherein the vent compartment is fluidly connected to at least one vent passageway.

Clause 213. The on-body device of any one of clauses 178 to 212, wherein the housing comprises at least one channel along the first surface.

Clause 214. The on-body device of clause 213, wherein the at least one channel comprises a first end connected to the vent inlet.

Clause 215. The on-body device of any one of clauses 178 to 214, wherein the first surface comprises a plurality of vent inlets.

Clause 216. The on-body device of any one of clauses 178 to 215, wherein the second surface comprises a plurality of vent outlets.

Clause 217. The on-body device of any one of clauses 178 to 214, wherein the on-body device comprises a plurality of vent passageways, a plurality of vent inlets, and a plurality of vent outlets.

Clause 218. The on body-unit of clause 217, wherein each vent passageway of the plurality of vent passageways extends from a vent inlet of the plurality of vent inlets to a vent outlet of the plurality of vent outlets.

Clause 219. The on body-unit of clause 217, wherein no two vent passageways of the plurality of vent passageways share a vent inlet of the plurality of vent inlets nor a vent outlet of the plurality of vent outlets.

Clause 220. The on-body device of any one of clauses 217 to 219, wherein the on-body device comprises two, three, four, five, six, seven, or eight vent passageways.

Clause 221. The on-body device of any one of clauses 217 to 219, wherein the on-body device comprises between two and ninety vent passageways.

Clause 222. The on-body device of any one of clauses 178 to 221, further comprising a moisture permeable seal.

Clause 223. The on-body device of clause 222, wherein the moisture permeable seal is at least partially within a vent passageway between the first surface and the second surface.

Clause 224. The on-body device of clause 223, wherein the moisture permeable seal is located entirely within the vent passageway.

Clause 225. The on-body device of any one of clauses 222 to 224, wherein the first surface is a lower base surface of the housing and the second surface is above the first surface in a position relatively farther from a body of the user, wherein the vent passageway extends between the lower base surface and the second surface.

Clause 226. The on-body device of clause 225, wherein the moisture permeable seal is located at an upper location, an intermediate location, or a lower location within the vent passageway.

Clause 227. The on-body device of any one of clauses 223 to 226, wherein the moisture permeable seal has a thickness that is 50% or less of an entire length of the vent passageway.

Clause 228. The on-body device of any one of clauses 223 to 226, wherein the moisture permeable seal has a thickness that is 1%-35% of an entire length of the vent passageway.

Clause 229. The on-body device of any one of clauses 223 to 226, wherein the moisture permeable seal has a thickness that is 1%-25% of an entire length of the passageway.

Clause 230. The on-body device any one of clauses 223 to 226, wherein the housing comprises a recess in a wall of the passageway, and wherein at least a portion of the moisture permeable seal is in the recess.

Clause 231. The on-body device of clause 230, wherein the recess is annular.

Clause 232. The on-body device of any one of clauses 223 to 231, wherein the housing comprises a projection from a wall of the vent passageway into an interior of the vent passageway, and wherein at least a portion of the moisture permeable seal abuts the projection.

Clause 233. The on-body device of clause 232, wherein the projection is annular.

Clause 234. The on-body device of any one of clauses 223 to 233, wherein the moisture permeable seal is a first moisture permeable seal, the on-body device further comprising a second moisture permeable seal configured to allow passage of water vapor through the vent passageway.

Clause 235. The on-body device of clause 234, wherein the first moisture permeable seal is located at an upper location within the passageway and the second moisture permeable seal is located at a lower location within the passageway.

Clause 236. The on-body device of any one of clauses 222 to 233, wherein the moisture permeable seal comprises a flange and a columnar portion.

Clause 237. The on-body device of clause 236, wherein the flange and columnar portion are located within the passageway, such that an end surface of the columnar portion is flush with a surface of the housing.

Clause 238. The on-body device of clause 236, wherein the moisture permeable seal further comprises a head portion configured to reside outside of the passageway and abut a surface of the housing.

Clause 239. The on-body device of clause 222, wherein the moisture permeable seal is configured as a liner.

Clause 240. The on-body device of clause 239, wherein the liner comprises adhesive.

Clause 241. The on-body device of clause 240, wherein the adhesive is configured to attach the liner to the second surface of the housing such that the liner covers the vent outlet.

Clause 242. The on-body device of clause 241, wherein the second surface of the housing is an upper surface configured to face away from a body of a wearer of the on-body device.

Clause 243. The on-body device of any one of 239 to 242, wherein the moisture permeable seal is configured as a sticker.

Clause 244. The on-body device of clause 222, wherein the first surface is a lower base surface and the second surface is above the first surface in a position relatively farther from a body of the user, wherein the vent passageway extends between the lower base surface and the second surface, and wherein the moisture permeable seal is configured as a cap for the opening in the second surface.

Clause 245. The on-body device of clause 244, wherein the cap comprises a roof member and a sidewall.

Clause 246. The on-body device of clause 245, wherein the sidewall comprises a first retention feature.

Clause 247. The on-body device of clause 246, wherein the first retention feature is configured to interlock with a second retention feature in a receiving space within the housing.

Clause 248. The on-body device of any one of clauses 244 to 247, wherein the cap is configured to be manually attached to the housing by a user.

Clause 249. The on-body device of clause 222, wherein the moisture permeable seal comprises a columnar portion configured to reside within the vent passageway and a head portion configured to abut the first surface of the housing.

Clause 250. The on-body device of clause 249, wherein the adhesive patch holds the moisture permeable seal against the first surface.

Clause 251. The on-body device of any of clauses 222 to 250, wherein the moisture permeable seal comprises a moisture permeable material.

Clause 252. The on-body device of clause 251, wherein the moisture permeable seal comprises silicone.

Clause 253. The on-body device of any one of clauses 222 to 252, wherein the moisture permeable seal is configured to allow passage of water vapor therethrough and block passage of liquid water through the passageway.

Clause 254. The on-body device of any one of clauses 222 to 253, wherein the moisture permeable seal is configured to allow accumulated moisture to exit a region between the on-body device housing and a body of the user by evaporation into a vapor and then migration through the moisture permeable seal into an ambient external environment away from the user's body.

Clause 255. The on-body device of any one of clauses 222 to 254, wherein the moisture permeable seal comprises two or more moisture permeable materials, or a moisture permeable material and a liquid permeable material.

Clause 256. The on-body device of any one of clauses 222 to 255, wherein the moisture permeable seal is fibrous.

Clause 257. The on-body device of any one of clauses 222 to 256, wherein the moisture permeable seal comprises perforations having a diameter between 10 and 1000 microns.

Clause 258. The on-body device of any one of clauses 222 to 256, wherein the moisture permeable seal comprises perforations having a diameter in at least one of the following ranges: between 100 and 700 microns; between 200 and 600 microns; and between 300 and 500 microns.

Clause 259. The on-body device of any one of clauses 258 to 257, wherein the moisture permeable seal comprises a hydrophilic material or material bearing a hydrophilic coating.

Clause 260. The on-body device of any one of clauses 222 to 259, wherein the on-body device has a Moisture Vapor Transmission Rate (MVTR) of 35 grams per square meter per hour (g/m2/hr) or more.

Clause 261. The on-body device of any one of clauses 178 to 260, further comprising a solid foam.

Clause 262. The on-body device of clause 261, wherein the solid foam is a foam seal.

Clause 263. The on-body device of any one of clauses 261 to 262, wherein the solid foam is an open-celled foam, a closed-celled foam, or a combination thereof.

Clause 264. The on-body device of any one of clauses 261 to 263, wherein the solid foam is configured to impede significant or total liquid water ingress while allowing moisture vapor and limited amounts of liquid to pass through.

Clause 265. The on-body device of any one of clauses 261 to 264, wherein the solid foam comprises at least one of reticulated polyurethane foam, hydrophobic treated open-cell foam, and silicone open-cell foam.

Clause 266. The on-body device of any one of clauses 261 to 265, wherein the solid foam is located in a vent passageway of the housing between the first surface and the second surface.

Clause 267. The on-body device of clause 266, wherein the solid foam is in contact with the adhesive patch.

Clause 268. The on-body device of any one of clauses 266 to 267, wherein the solid foam fills an entire length of the passageway.

Clause 269. The on-body device of any one of clauses 266 to 267, wherein the solid foam fills only a portion of the passageway.

Clause 270. The on-body device of clause 269, wherein the portion of the passageway filled by the solid foam has a length that is in at least one of the following ranges: 25-35% of the length of the passageway, 35-50% of the length of the passageway, 35-75% of the length of the passageway, 5-15% of the length of the passageway, or 15-25% of the length of the passageway.

Clause 271. The on-body device of clause 270, wherein the solid foam is located in an upper, intermediate, or lower location in the passageway.

Clause 272. The on-body device of any one of clauses 222 to 260, further comprising a solid foam in contact with the moisture permeable seal.

Clause 273. The on-body device of clause 272, further comprising a solid foam seal in a same passageway as the moisture permeable seal.

Clause 274. The on-body device of clause 273, wherein the moisture permeable seal is located at the vent outlet.

Clause 275. The on-body device of clause 273, wherein the moisture permeable seal is located at the vent inlet.

Clause 276. The on-body device of clause 273, wherein the moisture permeable seal is a first moisture permeable seal, the housing comprising a second moisture permeable seal in the same passageway as the first moisture permeable seal, with the solid foam located therebetween.

Clause 277. The on-body device of clause 273, wherein the solid foam is a first solid foam, the housing comprising a second solid foam in the same passageway as the first solid foam, with the moisture permeable seal located therebetween.

Clause 278. The on-body device of any one of clauses 178 to 277, further comprising a fibrous seal configured to transport liquid by capillary action.

Clause 279. The on-body device of clause 278, wherein the adhesive patch is different from the fibrous seal.

Clause 280. The on-body device of any one of clauses 178 to 279, wherein the analyte is glucose.

Clause 281. The on-body device of any one of clauses 178 to 279, wherein the analyte is selected from the list consisting of: glucose, one or more ketones, lactate, and oxygen.

Clause 282. The on-body device of any one of clauses 178 to 279, wherein the analyte is selected from the list consisting of: ammonium, asparagine, aspartate, cholesterol, choline, creatinine, diaphorase inhibitors, ethanol, glucose, glutamate, glycerol, one or more ketones, lactate, lactose, oxygen, phosphate, potassium, pyruvate, and uric acid.

Clause 283. The on-body device of any one of clauses 178 to 279, wherein the analyte is a first analyte, and the sensor is further configured to measure a level of a second analyte of the user.

Clause 284. The on-body device of clause 283, wherein the first analyte is glucose and the second analyte is a ketone.

Clause 285. The on-body device of clause 283, wherein the first and second analytes are selected from the list consisting of: ammonium, asparagine, aspartate, cholesterol, choline, creatinine, diaphorase inhibitors, ethanol, glucose, glutamate, glycerol, one or more ketones, lactate, lactose, oxygen, phosphate, potassium, pyruvate, and uric acid.

Clause 286. The on-body device of any one of clauses 178 to 285, wherein the on-body device has a designed wear duration of 15 to 30 days.

Clause 287. The on-body device of any one of clauses 178 to 285, wherein the on-body device has a designed wear duration of 31 to 45 days.

Clause 288. The on-body device of any one of clauses 178 to 285, wherein the on-body device has a designed wear duration that is 45 days or greater.

Clause 289. A method of monitoring an analyte of a user, comprising: applying an on-body device to a body of the user; and monitoring a level of the analyte with a sensor in communication with the on-body device, wherein the sensor is either a wholly in vivo sensor or a wholly ex vivo sensor, and wherein the on-body device is configured in accordance with any one of clauses 178 to 288.

Clause 290. The method of clause 289, wherein monitoring the level of the analyte with the on-body device comprises monitoring the level of the analyte with the on-body device for a wear duration without removing the on-body device from the body of the user.

Clause 291. The method of clause 290, wherein the wear duration is 7 days or greater.

Clause 292. The method of clause 290, wherein the wear duration is 14 days or greater.

Clause 293. The method of clause 290, wherein the wear duration is 21 days or greater.

Clause 294. The method of clause 290, wherein the wear duration is 30 days or greater.

Clause 295. The method of clause 290, wherein the wear duration is 45 days or greater.

Clause 296. The method of clause 290, wherein the wear duration is between 7 days and 60 days.

Clause 297. The method of any one of clauses 290 to 296, wherein the analyte is a first analyte, the method further comprising monitoring a level of a second analyte with the on-body device.

Clause 298. The method of any one of clauses 290 to 296, wherein the analyte is a first analyte, the method further comprising monitoring a level of a second analyte with the on-body device for the wear duration.

Clause 299. The method of any one of clauses 297 to 298, further comprising transmitting, from the on-body device to a receiver unit, data indicative of the monitored level of the first analyte and the monitored level of the second analyte.

Clause 300. The method of any one of clauses 289 to 296, further comprising transmitting, from the on-body device to a receiver unit, data indicative of the monitored level of the analyte.

Clause 301. The method of any one of clauses 289 to 300, wherein the sensor is a wholly in vivo sensor, and applying the on-body device to the body of the user comprises applying the on-body device to the body of the user in a position over the wholly in vivo sensor.

Clause 302. The method of any one of clauses 300 to 301, wherein the data indicative of the monitored level of the analyte is transmitted according to a Bluetooth or Bluetooth Low Energy (BLE) protocol.

Clause 303. The method of any one of clauses 300 to 302, wherein the data indicative of the monitored level of the analyte is transmitted according to a Near Field Communication (NFC) protocol.

Clause 304. The method of any one of clauses 300 to 303, wherein the receiver unit is a smart phone.

Clause 305. The method of any one of clauses 300 to 303, wherein the receiver unit is a dedicated receiver unit.

Clause 306. The method of any one of clauses 300 to 303, wherein the receiver unit is a smart watch.

Clause 307. The method of any one of clauses 289 to 298, further comprising transmitting, from the on-body device to a plurality of receiver units, data indicative of the monitored level of the analyte, wherein the plurality of receiver units are each in an authenticated state at the on-body device, and wherein the plurality of receiver units comprise a smart phone and a smart watch.

Clause 308. The method of clause 301, further comprising wirelessly transmitting data indicative of the level of the analyte from the wholly in vivo sensor to the on-body device.

Clause 309. The method of clause 308, wherein the data indicative of the monitored level of the analyte is transmitted from the wholly in vivo sensor according to a Bluetooth or Bluetooth Low Energy (BLE) protocol.

Clause 310. The method of clause 308, wherein the data indicative of the monitored level of the analyte is transmitted from the wholly in vivo sensor according to a Near Field Communication (NFC) protocol.

Clause 311. An insertion system, comprising: the on-body device for monitoring the level of the analyte of the user according to any one of clauses 178 to 288; and an applicator configured to position the on-body device on the skin of the user.

Clause 312. The system of clause 311, further comprising a receiver unit configured to receive data indicative of the analyte level from the on-body device.

Clause 313. The system of clause 312, wherein the receiver unit is configured to receive the data indicative of the monitored level of the analyte according to a Bluetooth or Bluetooth Low Energy (BLE) protocol.

Clause 314. The system of any one of clauses 311 and 312, wherein the receiver unit is configured to receive the data indicative of the monitored level of the analyte according to a Near Field Communication (NFC) protocol.

Clause 315. The system of clause 311, further comprising a plurality of receiver units configured to receive data indicative of the monitored level of the analyte from the on-body device, wherein the on-body device is configured to authenticate the plurality of receiver units, and wherein the plurality of receiver units comprise a smart phone and a smart watch.

Clause 316. A method of manufacturing an on-body device for monitoring an analyte level of a user, comprising: providing a housing comprising at least one passageway through the housing; and injecting a moisture permeable material into the passageway, wherein the moisture permeable materials forms a seal configured to allow passage of water vapor through the passageway.

Clause 317. The method of clause 316, further comprising: coupling sensor electronics with the housing; and coupling an analyte sensor to the sensor electronics, wherein the analyte sensor is configured to monitor an analyte level of the user.

Clause 318. The method of any one of clauses 316 to 317, further comprising: positioning either a foam seal or a fibrous seal in the at least one passageway.

Clause 319. A method of manufacturing an on-body device for monitoring an analyte level of a user, comprising: providing a housing comprising at least one passageway through the housing; and inserting a moisture permeable seal into the passageway or into a position that covers the passageway, wherein the moisture permeable seal is configured to allow passage of water vapor through the passageway.

Clause 320. The method of clause 319, further comprising: coupling sensor electronics with the housing; and coupling an analyte sensor to the sensor electronics, wherein the analyte sensor is configured to monitor an analyte level of the user.

Clause 321. The method of any one of clauses 319 to 320, further comprising: positioning either a foam seal or a fibrous seal in the at least one passageway.

Clause 322. An on-body device for monitoring an analyte level of a user, comprising: a housing, comprising: a first surface configured for placement such that it faces adjacent skin of the user; a second surface; and a vent passageway configured to allow passage of water vapor from the first surface to the second surface; sensor electronics, wherein the housing is configured to retain the sensor electronics; and a sensor, comprising: a first portion configured to be in electrical contact with the sensor electronics; and a second portion configured to be positioned in the body of the user for monitoring a level of an analyte of the user; wherein the sensor is configured to extend through an aperture in the first surface for positioning the second portion of the sensor in the body of the user, and wherein the on-body device is configured such that no portion of the sensor resides in the vent passageway during sensor operation.

Clause 323. The on-body device of clause 322, further comprising at least one of a moisture permeable seal, a foam seal, or a fibrous seal.

Clause 324. An on-body device, comprising: electronics configured to receive a signal from a sensor, the signal indicative of a level of an analyte of a user; and a housing configured to retain the electronics, wherein the housing is adapted to permit egress of moisture from an area adjacent the housing.

Clause 325. The on-body device of clause 324, wherein the housing comprises a base having a first surface for facing a skin surface of the user.

Clause 326. The on-body device of clause 325, wherein the base comprises a first region, and wherein the first region comprises an aperture in the housing.

Clause 327. The on-body device of clause 326, wherein the first region of the base is spaced apart from a periphery of the base.

Clause 328. The on-body device of clauses 326 or 327, wherein the aperture is positioned in the center of the first region.

Clause 329. The on-body device of any of clauses 326 to 328, wherein the first region comprises a recessed region of the base.

Clause 330. The on-body device of clause 325 or any preceding clause when directly or indirectly dependent thereon, wherein the housing comprises at least one channel along the first surface, wherein the at least one channel is configured to permit egress of moisture from an area adjacent the first surface.

Clause 331. The on-body device of any of clause 330, wherein the at least one channel is formed in the base of the housing.

Clause 332. The on-body device of clause 330 or 331, wherein the at least one channel comprises one or more first channels and/or one or more second channels.

Clause 333. The on-body device of clause 332, wherein each of the one or more first channels comprise a first end arranged at a periphery of the base.

Clause 334. The on-body device of clause 333 when directly or indirectly dependent on clause 326, wherein at least a portion of the one or more first channels comprises a second end arranged at the first region of the base.

Clause 335. The on-body device of any of clauses 332 to 334, wherein each of the one or more second channels comprises a first end arranged at the periphery of the base and a second end arranged at the periphery of the base.

Clause 336. The on-body device of clauses 332 or 335, wherein each of the one or more second channels comprises a curved shape.

Clause 337. The on-body device of any of clauses 332 to 336, wherein each of the one or more second channels is arranged to intersect at least one of the first channels.

Clause 338. The on-body device of any of clauses 332 to 337, wherein the depth of each of the one or more first channels and/or each of the one or more second channels varies along at least a portion of the length of the respective channel.

Clause 339. The on-body device of clause 338, wherein the depth of each of the one or more first channels decreases as each respective first channel progresses toward the first region of the base.

Clause 340. The on-body device of clause 339, wherein the depth of each of the one or more first channels decreases to zero as the respective first channel progresses toward the first region of the base.

Clause 341. The on-body device of clauses 339 or 340, wherein the depth of each of the one or more first channels decreases as each respective channel progresses toward the first region of the base until each respective first channel bottom meets the first surface of the base.

Clause 342. The on-body device of any of clauses 338 to 341, wherein the portion of the length of the respective channel is 1% or greater of the length of the respective channel, 2% or greater of the length of the respective channel, 3% or greater of the length of the respective channel, 5% or greater of the length of the respective channel, 8% or greater of the length of the respective channel, 10% or greater of the length of the respective channel, 15% or greater of the length of the respective channel, 20% or greater of the length of the respective channel, 25% or greater of the length of the respective channel, 30% or greater of the length of the respective channel, 50% or greater of the length of the respective channel, 75% or greater of the length of the respective channel, or 95% or greater of the length of the respective channel.

Clause 343. The on-body device of any of clauses 338 to 342, wherein each of the one or more first channels and/or each of the one or more second channels tapers in depth.

Clause 344. The on-body device of clause 334 or any preceding clause when directly or indirectly dependent thereon, wherein each of the one or more first channels includes a tapered edge and/or a beveled edge where the respective channel meets the first region.

Clause 345. The on-body device of any of clauses 338 to 344, wherein the depth of each of the one or more first channels and/or each of the one or more second channels varies in a stepped fashion.

Clause 346. The on-body device of any of clauses 338 to 345, wherein the depth of each of the one or more first channels and/or each of the one or more second channels decreases with a radiused sloping edge.

Clause 347. The on-body device of any of clauses 338 to 346, wherein a depth of each of the one or more first channels and/or each of the one or more second channels increases as the channel progresses towards the peripheral edge of the base.

Clause 348. The on-body device of any of clauses 338 to 347, wherein each of the one or more first channels and/or each of the one or more second channels has a maximum depth at or adjacent to the peripheral edge of the base.

Clause 349. The on-body device of any of clauses 332 to 348, wherein the width of each of the one or more first channels and/or each of the one or more second channels tapers as the respective channel progresses towards a peripheral edge of the housing.

Clause 350. The on-body device of any of clauses 332 to 349, wherein the width of each of the one or more first channels and/or each of the one or more second channels narrows as the respective channel progresses towards a peripheral edge of the housing.

Clause 351. The on-body device of clauses 349 or 350, wherein the width of each of the one or more first channels and/or each of the one or more second channels tapers along a portion of the length of the respective channel at or adjacent a peripheral edge of the base.

Clause 352. The on-body device of any of clauses 332 to 351, wherein each of the one or more first channels and/or each of the one or more second channels terminates along a peripheral edge of the housing in the shape of a slit.

Clause 353. The on-body device of any of clauses 332 to 352, wherein each of the one or more first channels and/or each of the one or more second channels has a first width at a first location and a second width at a second location, and wherein the first width transitions to the second width in a stepped or continuous fashion.

Clause 354. The on-body device of clause 353, wherein a first width of each of the one or more first channels and/or each of the one or more second channels at the first location is less than a second width of the respective channel at the second location.

Clause 355. The on-body device of clauses 353 or 354, wherein a first depth of each of the one or more first channels and/or each of the one or more second channels at the first location is less than a second depth of the channel at the second location.

Clause 356. The on-body device of any of clauses 332 to 355, wherein each of the one or more first channels and/or each of the one or more second channels has a first lengthwise span adjacent a peripheral edge of the housing and a second lengthwise span located relatively farther from the peripheral edge than the first lengthwise span.

Clause 357. The on-body device of clause 356 when dependent on clause 353, wherein the first lengthwise span maintains the first width in constant fashion and the second lengthwise span maintains the second width in constant fashion.

Clause 358. The on-body device of clause 356 when dependent on clause 353, wherein the first lengthwise span varies in width across a range that includes the first width, and the second lengthwise span maintains the second width in constant fashion.

Clause 359. The on-body device of clause 356 when dependent on clause 353, wherein the first lengthwise span maintains the first width in constant fashion, and the second lengthwise span varies in width across a range that includes the second width.

Clause 360. The on-body device of clause 356 when dependent on clause 353, wherein the first lengthwise span varies in width across a range that includes the first width, and the second lengthwise span varies in width across a range that includes the second width.

Clause 361. The on-body device of any of clauses 356 to 360, wherein the first lengthwise span gradually transitions in width to the second lengthwise span.

Clause 362. The on-body device of any of clauses 356 to 361, wherein the first lengthwise span and the second lengthwise span of each of the one or more first channels are separated due to intersection with one or more of the second channels.

Clause 363. The on-body device of any of clauses 332 to 355, wherein each of the one or more first channels and/or each of the one or more second channels has a non-constant width along an entire length thereof.

Clause 364. The on-body device of any of clauses 332 to 363, wherein a width of each of the one or more second channels is constant along its entire length.

Clause 365. The on-body device of any of clauses 330 to 364, wherein each of the at least one channels comprises a first sidewall, a second sidewall, and a channel bottom that is distal to the first surface.

Clause 366. The on-body device of clause 365, wherein the first sidewall and the second sidewall extend between the first surface and the channel bottom.

Clause 367. The on-body device of clauses 365 or 366, wherein the depth of each of the at least one channels is a distance between the respective channel bottom and the first surface.

Clause 368. The on-body device of any of clauses 365 to 367, wherein the depth of each of the at least one channels is a distance between the respective channel bottom and the first surface of the base along an axis normal to a plane of the first surface of the base.

Clause 369. The on-body device of any of clauses 365 to 368, wherein the first sidewall meets the first surface at a first intersection, the second sidewall meets the first surface at a second intersection directly opposite the first intersection, and the width of the channel is a distance between the first intersection and the second intersection.

Clause 370. The on-body device of clause 368, wherein the channel comprises a fillet or a chamfer at the first intersection and/or the second intersection.

Clause 371. The on-body device of any of clauses 332 to 370, wherein the base comprises an interior surface region and a peripheral surface region located between the interior region and the peripheral edge.

Clause 372. The on-body device of clause 371, wherein each of the one or more first channels extends across the base in both the interior surface region and the peripheral surface region, wherein each of the one or more first channels has a narrower width in the peripheral surface region than in the interior surface region.

Clause 373. The on-body device of clause 332 or any preceding clause when directly or indirectly dependent thereon, wherein a first subset of the one or more second channels is closer to the peripheral edge of the housing than a second subset of the one or more second channels, and wherein the first subset have a narrower width than the second subset.

Clause 374. The on-body device of any of clauses 332 to 372, wherein the one or more second channels comprises an arc.

Clause 375. The on-body device of clause 374, wherein the arc is an arc of a circle.

Clause 376. The on-body device of clause 374, wherein the arc is an arc of an ellipse.

Clause 377. The on-body device of clause 336 or any preceding clause when directly or indirectly dependent thereon, wherein the curved shape has a constant radius of curvature.

Clause 378. The on-body device of clause 336 or any preceding clause when directly or indirectly dependent thereon, wherein the curved shape is C-shaped.

Clause 379. The on-body device of clause 336 or any preceding clause when directly or indirectly dependent thereon, wherein the curved shape is U-shaped.

Clause 380. The on-body device of clause 336 or any preceding clause when directly or indirectly dependent thereon, wherein the curved shape comprises a first curved portion and a second curved portion.

Clause 381. The on-body device of clause 380, wherein the first curved portion comprises a first focal point and the second curved portion comprises a second focal point.

Clause 382. The on-body device of clause 380 or 381, wherein the first curved portion has a first radius of curvature and the second curved portion has a second radius of curvature.

Clause 383. The on-body device of clause 332 or any preceding clause when directly or indirectly dependent thereon, wherein the one or more second channels does not comprise any corner that has an angle of less than 135°.

Clause 384. The on-body device of clause 332 or any preceding clause when directly or indirectly dependent thereon, wherein the one or more second channels does not comprise any bend that has a bend radius of less than 2 mm.

Clause 385. The on-body device of any preceding clause when directly or indirectly dependent on clauses 326 and 336, wherein the curved shape of at least some of the one or more second channels comprises an interior side of the curve arranged towards the first region of the base.

Clause 386. The on-body device of clause 385, wherein a focal point of the curved shape of the at least some of the one or more second channels is the aperture.

Clause 387. The on-body device of clause 385 or 386, wherein the aperture is arranged off-centre in the base of the housing.

Clause 388. The on-body device of any of clauses 385 to 387, wherein the aperture is arranged on a first side of the first surface of the base relative to a centre of the base, and wherein at least a portion of the one or more second channels is arranged on a second side of the first surface of the base opposite to the first side of the first surface of the base relative to the centre of the base.

Clause 389. The on-body device of any of clauses 385 to 388, wherein the aperture in the base of the housing defines a sensor passageway for receiving the sensor and/or for receiving a sharp for positioning the sensor.

Clause 390. The on-body device of clause 332 or any preceding clause when directly or indirectly dependent thereon, wherein the base further comprises one or more third channels comprising a first end arranged at the periphery of the base and extending to a second end connected to the at least one second channel.

Clause 391. The on-body device of clause 332 or any preceding clause when directly or indirectly dependent thereon, wherein the one or more first channels are straight channels.

Clause 392. The on-body device of clause 332 or any preceding clause when directly or indirectly dependent thereon, wherein each of the one or more first channels extend radially from a position on the first surface of the base that is offset from the peripheral edge of the housing.

Clause 393. The on-body device of clause 392, wherein the position corresponds to the position of the aperture in the housing.

Clause 394. The on-body device of clause 332 or any preceding clause when directly or indirectly dependent thereon, wherein the one or more first channels extend radially between a periphery of the base and an interior region of the base.

Clause 395. The on-body device of clause 332 or any preceding clause when directly or indirectly dependent thereon, wherein each of the one or more first channels and/or each of the one or more second channels have a cross-sectional profile that is U-shaped, V-shaped, or three-sided.

Clause 396. The on-body device of clause 395, wherein each of the one or more first channels and/or each of the one or more second channels has a three-sided cross-sectional profile with two interior right angles.

Clause 397. The on-body device of clause 395, wherein each of the one or more first channels and/or each of the one or more second channels has a three-sided cross-sectional profile with two interior angles between 45 and 90 degrees.

Clause 398. The on-body device of clause 395, wherein each of the one or more first channels and/or each of the one or more second channels has a three-sided cross-sectional profile with two interior angles between 60 and 90 degrees.

Clause 399. The on-body device of any of clauses 395 to 398, wherein each of the one or more first channels and/or each of the one or more second channels has a three-sided cross-sectional profile with a fillet or a chamfer at a side intersection.

Clause 400. The on-body device of clause 332 or any preceding clause when directly or indirectly dependent thereon, wherein each of the one or more first channels and/or each of the one or more second channels has a maximum depth that is greater than 50 μm.

Clause 401. The on-body device of clause 332 or any preceding clause when directly or indirectly dependent thereon, wherein each of the one or more first channels and/or each of the one or more second channels has a maximum depth between 100 μm and 400 μm.

Clause 402. The on-body device of clause 332 or any preceding clause when directly or indirectly dependent thereon, wherein each of the one or more first channels and/or each of the one or more second channels has a maximum depth between 100 μm and 200 μm.

Clause 403. The on-body device of clause 332 or any preceding clause when directly or indirectly dependent thereon, wherein each of the one or more first channels and/or each of the one or more second channels has a maximum depth of around 100 μm.

Clause 404. The on-body device of clause 332 or any preceding clause when directly or indirectly dependent thereon, wherein each of the one or more first channels and/or each of the one or more second channels has a width between 400 μm and 1000 μm.

Clause 405. The on-body device of clause 332 or any preceding clause when directly or indirectly dependent thereon, wherein each of the one or more first channels and/or each of the one or more second channels has a width between 600 μm and 900 μm.

Clause 406. The on-body device of clause 332 or any preceding clause when directly or indirectly dependent thereon, wherein each of the one or more first channels and/or each of the one or more second channels has a width between 700 μm and 800 μm.

Clause 407. The on-body device of clause 332 or any preceding clause when directly or indirectly dependent thereon, wherein each of the one or more first channels and/or each of the one or more second channels has a width of around 800 μm.

Clause 408. The on-body device of clause 332 or any preceding clause when directly or indirectly dependent thereon, wherein each of the one or more first channels and/or each of the one or more second channels has a width of around 800 μm and a maximum depth of around 100 μm.

Clause 409. The on-body device of clause 332 or any preceding clause when directly or indirectly dependent thereon, wherein each of the one or more first channels and/or each of the one or more second channels has a width to maximum depth ratio of around 8:1.

Clause 410. The on-body device of clause 332 or any preceding clause when directly or indirectly dependent thereon, wherein each of the one or more first channels and/or each of the one or more second channels has a rectangular cross-section.

Clause 411. The on-body device of any of clauses 400 to 403 or 408, wherein the maximum depth is measured between the bottom of the respective channel and first surface of the base along a direction normal to the first surface of the base.

Clause 412. The on-body device of any of clauses 404 to 408, wherein the width of the channel is measured between opposing sidewalls of the respective channel, in the plane of the first surface of the base and normal to the lengthways axis of the respective channel.

Clause 413. The on-body device of clause 332 or any preceding clause when directly or indirectly dependent thereon, wherein the first surface of the base comprises a plurality of proximal portions between the one or more first channels and/or the one or more second channels.

Clause 414. The on-body device of clause 413, wherein the plurality of proximal portions define a proximal surface of the base of the housing.

Clause 415. The on-body device of clause 413 or 414, wherein the plurality of proximal portions comprise protrusions of the base to define boundaries of the one or more first channels and/or the one or more second channels.

Clause 416. The on-body device of any of clauses 413 to 415, wherein the proximal portions are integrally formed with the base of the housing.

Clause 417. The on-body device of any of clauses 413 to 416, wherein the proximal portions define a contact surface area of between 30% and 80% of the proximal surface of the base, optionally wherein the proximal portions define a contact surface area of between 50% and 60% of the proximal surface of the base, optionally wherein the proximal portions define a contact surface area of around 55% of the proximal surface of the base.

Clause 418. The on-body device of any of clauses 413 to 417, wherein the proximal portions comprise a first proximal portion and a second proximal portion each having a contact surface for contact with the adhesive patch, wherein the first proximal portion and the second portion are defined, at least in part, by channels of the one or more first channels and/or the one or more second channels, and wherein the contact surface of the first proximal portion is 1.25 times as large or more in surface area than the contact surface of the second proximal portion, optionally wherein the contact surface of the first proximal portion is two times as large or more in surface area than the contact surface of the second proximal portion, optionally wherein the contact surface of the first proximal portion is three times as large or more in surface area than the contact surface of the second proximal portion, optionally wherein the first proximal portion does not extend to the peripheral edge of the base.

Clause 419. The on-body device of clause 418, wherein the first proximal portion has a contact surface with a surface area that is 5% or greater of the proximal surface of the base, optionally wherein the first proximal portion has a contact surface with a surface area that is 7% or greater of the proximal surface of the base, optionally wherein the first proximal portion has a contact surface with a surface area that is 10% or greater of the proximal surface of the base.

Clause 420. The on-body device of any of clauses 413 to 419, wherein the proximal portions comprise a moisture-permeable material.

Clause 421. The on-body device of clause 332 or any preceding clause when directly or indirectly dependent thereon, wherein the one or more first channels and/or the one or more second channels comprise grooves formed in the base of the housing.

Clause 422. The on-body device of clause 330 or any preceding clause when directly or indirectly dependent thereon, wherein the housing comprises: at least one vent inlet in the first surface; at least one vent outlet in a second surface of the housing, the second surface opposing the first surface, wherein the vent inlet is fluidically connected to the vent outlet through the housing to allow the passage of water vapor from the first surface to the second surface.

Clause 423. The on-body device of clause 422, wherein the at least one vent inlet is spaced away from a periphery of the first surface.

Clause 424. The on-body device of clause 422 or 423, wherein the at least one vent outlet is aligned with the at least one vent inlet along an axis perpendicular to the first surface.

Clause 425. The on-body device of any of clauses 422 to 424, wherein the at least one vent outlet is smaller than the at least one vent inlet.

Clause 426. The on-body device of any of clauses 422 to 425, wherein the at least one vent outlet comprises a width of less than 1 mm.

Clause 427. The on-body device of any of clauses 422 to 426, wherein the housing comprises a vent passageway between the at least one vent inlet and the respective at least one vent outlet.

Clause 428. The on-body device of clause 427, wherein each vent passageway has a longitudinal axis that is normal to at least a portion of the first surface at the respective vent inlet.

Clause 429. The on-body device of clause 427, wherein each vent passageway has a longitudinal axis that is angled with respect to at least a portion of the first surface at the respective vent inlet.

Clause 430. The on-body device of clause 427 or 429, wherein the longitudinal axis of each vent passageway is angled from the respective vent inlet to the respective vent outlet in a direction towards a periphery of the housing.

Clause 431. The on-body device of clause 427 or 429, wherein the longitudinal axis of each vent passageway is angled from the respective vent inlet to the respective vent outlet in a direction away from a periphery of the housing.

Clause 432. The on-body device of any of clauses 427 to 431, wherein each of the at least one vent inlets is wider than the respective at least one vent outlet, and the respective vent passageway comprises a tapered width portion.

Clause 433. The on-body device of any of clauses 427 to 431, wherein each of the at least one vent outlets is wider than the respective vent inlet, and the respective vent passageway comprises a tapered width portion.

Clause 434. The on-body device of any of clauses 427 to 433, wherein each of the at least one vent inlets is wider than the respective vent outlet, and the respective vent passageway comprises a step transition in width from the vent inlet to the vent outlet.

Clause 435. The on-body device of any of clauses 427 to 434, wherein each vent passageway has a first portion that extends along a first longitudinal axis, and a second portion that extends along a second longitudinal axis, wherein the first longitudinal axis is transverse to the second longitudinal axis.

Clause 436. The on-body device of clause 435, wherein the first longitudinal axis is perpendicular to the second longitudinal axis.

Clause 437. The on-body device of any of clauses 427 to 436, wherein each vent passageway comprises a portion that extends along a curved longitudinal axis.

Clause 438. The on-body device of any of clauses 427 to 437, wherein each entire vent passageway extends along a curved longitudinal axis.

Clause 439. The on-body device of clause 423 or any of clauses 424 to 438 when directly or indirectly dependent on clause 423, wherein each of the at least one vent inlets is relatively farther from the periphery than the respective vent outlet.

Clause 440. The on-body device of clause 423 or any of clauses 424 to 438 when directly or indirectly dependent on clause 423, wherein each of the at least one vent inlets is relatively closer to the periphery than the respective vent outlet.

Clause 441. The on-body device of any of clauses 427 or any of clauses 428 to 440 when directly or indirectly dependent on clause 427, wherein each vent passageway extends through the electronics.

Clause 442. The on-body device of any of clause 441, wherein each vent passageway extends through an aperture in a printed circuit board of the electronics.

Clause 443. The on-body device of any of clauses 422 to 442, wherein the at least one channel comprises one or more fourth channels connected to the at least one vent inlet.

Clause 444. The on-body device of clause 443, wherein the one or more fourth channels terminate at or adjacent to the respective vent inlet.

Clause 445. The on-body device of clause 443, wherein the one or more fourth channels are fluidically connected to the at least one vent inlet.

Clause 446. The on-body device of clause 443 or 445, wherein the at least one vent inlet comprises a plurality of vent inlets, and wherein each of the one or more fourth channels comprises a first end arranged at a first vent inlet of the plurality of vent inlets and a second end arranged at a second vent inlet.

Clause 447. The on-body device of clause 446, wherein the plurality of vent inlets comprises a first, a second and a third vent inlet, wherein a first of the one or more fourth channels is connected to the first and second vent inlets, a second of the one or more fourth channels is connected to the second and third vent inlets, and a third of the one or more fourth channels is connected to the first and third vent inlets.

Clause 448. The on-body device of clause 447, wherein each of the one or more fourth channels is spaced away from a periphery of the base of the housing.

Clause 449. The on-body device of any of clauses of any of clauses 443 to 445, wherein the at least one vent inlet comprises a plurality of vent inlets, and wherein a fourth channel of the one or more fourth channels comprises a first end arranged at a first vent inlet of the plurality of vent inlets and a second end arranged at the first region of the base.

Clause 450. The on-body device of clause 449, wherein another of the one or more fourth channels comprises a first end arranged at a second vent inlet of the plurality of vent inlets, and a second end arranged at the first region.

Clause 451. The on-body device of clause 450, wherein another of the one or more fourth channels comprises a first end arranged at the first vent inlet and a second end arranged at a third vent inlet of the plurality of vent inlets.

Clause 452. The on-body device of clause 451, wherein another of the one or more fourth channels comprises a first end arranged at the second vent inlet and a second end arranged at the third vent inlet.

Clause 453. The on-body device of any of clauses 443 to 453, wherein each of the at least one vent inlets is formed in a bottom of a respective fourth channel.

Clause 454. The on-body device of any of clauses 443 to 453, wherein the one or more fourth channels are narrower than the vent inlets.

Clause 455. The on-body device of any of clauses 443 to 454, wherein the one or more fourth channels are wider than the vent inlets.

Clause 456. The on-body device of any of clauses 446 to 455, wherein the one or more fourth channels, the plurality of vent inlets and/or the first region are arranged in a shape, wherein the shape comprises: a line; a circle; an ellipse; an oval; a semi-circle; a triangle; a square; a pentagon; a hexagon; a heptagon; an octagon; or a star.

Clause 457. The on-body device of clause 456, wherein the vent inlets are positioned at vertices or corners of the respective shape.

Clause 458. The on-body device of any of clause 456 or 457, wherein the vent inlets are positioned along edges of the respective shape, the edges being defined by the fourth channels.

Clause 459. The on-body device of any of clauses 443 to 458, wherein the one or more fourth channels have a maximum depth between 100 μm and 400 μm.

Clause 460. The on-body device of any of clauses 443 to 458, wherein the one or more fourth channels have a maximum depth of around 100 μm.

Clause 461. The on-body device of any of clauses 443 to 460, wherein the one or more fourth channels have a width between 400 μm and 1000 μm.

Clause 462. The on-body device of any of clauses 443 to 460, wherein the one or more fourth channels have a width of around 800 μm.

Clause 463. The on-body device of any of clauses 443 to 462, wherein the one or more fourth channels have a U-shaped, V-shaped or three-sided cross-sectional shape.

Clause 464. The on-body device of any of clauses 443 to 463, wherein the one or more fourth channels have a curved shape.

Clause 465. The on-body device of any of clauses 443 to 463, wherein the one or more fourth channels are straight.

Clause 466. The on-body device of any of clauses 443 to 465, wherein the one or more fourth channels comprise a tapered edge, a beveled edge and/or a radiused sloping edge where the respective fourth channel connects to the respective vent aperture.

Clause 467. The on-body device of any of clauses 443 to 466, wherein the one or more fourth channels comprise a tapering or narrowing depth as the fourth channel progresses toward the vent aperture.

Clause 468. The on-body device of any of clauses 443 to 467, wherein the one or more fourth channels comprise a tapering or narrowing width along portions of the fourth channel that are closer to a peripheral edge of the base of the housing.

Clause 469. The on-body device of clause 427 or any of clauses 428 to 468 when directly or indirectly dependent on clause 427, further comprising a moisture permeable seal provided in each vent passageway.

Clause 470. The on-body device of clause 325 or any preceding clause when directly or indirectly dependent thereon, wherein the first surface of the base comprises a surface texture.

Clause 471. The on-body device of clause 470, wherein the surface texture facilitates the movement of moisture or liquid across a surface of the base.

Clause 472. The on-body device of clause 470 or 471, further comprising a flexible patch adhesively coupled with the base, wherein the surface texture facilitates adhesive coupling between the base and the flexible patch.

Clause 473. The on-body device of any of clauses 470 to 472, wherein the surface texture is substantially filled with adhesive.

Clause 474. The on-body device of any of clauses 470 to 473 when directly or indirectly dependent on clause 330, wherein the at least one channel is substantially not filled with adhesive.

Clause 475. The on-body device of any of clauses 470 to 474, wherein the surface texture comprises directional surface texture.

Clause 476. The on-body device of any of clauses 470 to 475, wherein at least a portion of the surface texture is oriented to facilitate movement of moisture or liquid towards a peripheral edge of the base.

Clause 477. The on-body device of any of clauses 470 to 476, wherein the surface texture has an average width and an average depth.

Clause 478. The on-body device of clause 477, wherein the average width of the surface texture is greater than the average depth of the surface texture.

Clause 479. The on-body device of clause 477, wherein the average width of the surface texture is two or more times the average depth of the surface texture.

Clause 480. The on-body device of clause 477, wherein the average width of the surface texture is three or more times the average depth of the surface texture.

Clause 481. The on-body device of any of clauses 477 to 480, wherein the surface texture has an average pitch that is three or more times the average depth of the surface texture.

Clause 482. The on-body device of any of clauses 470 to 481, wherein the surface texture comprises a plurality of surface grooves.

Clause 483. The on-body device of clause 482, wherein each one of the plurality of surface grooves has a cross-sectional profile that is U-shaped, V-shaped, or three-sided.

Clause 484. The on-body device of clause 482 or 483, wherein each of the plurality of surface grooves has a depth of between 42 and 57 μm, a width of between 420 and 570 μm, and a pitch of between 680 and 920 μm.

Clause 485. The on-body device of clause 482 or 483, wherein each of the plurality of surface grooves has a depth of around 50 μm, a width of around 600 μm, and a pitch of around 850 μm.

Clause 486. The on-body device of clause 482 or 483, wherein each of the plurality of surface grooves has a depth of between 21 and 29 μm, a width of between 150 and 200 μm, and a pitch of between 340 and 460 μm.

Clause 487. The on-body device of clause 482 or 483, wherein each of the plurality of surface grooves has a depth of around 40 μm, a width of around 100 μm, and a pitch of around 300 μm.

Clause 488. The on-body device of any of clauses 470 to 486, wherein at least a portion of the surface texture is on an area of the base comprising adhesive.

Clause 489. The on-body device of any of clauses 475 to 488, wherein the surface texture is oriented within 15 degrees of a common direction.

Clause 490. The on-body device of any of clauses 475 to 488, wherein the surface texture is oriented in substantially the same direction.

Clause 491. The on-body device of any of clauses 475 to 488, wherein the directionality of the surface texture is oriented straight.

Clause 492. The on-body device of any of clauses 475 to 488, wherein the directionality of the surface texture follows a curved direction.

Clause 493. The on-body device of any of clauses 475 to 488, wherein the directionality of the surface texture is circular.

Clause 494. The on-body device of any of clauses 475 to 488, wherein the directionality of the surface texture is elliptical, triangular or square.

Clause 495. The on-body device of any of clauses 475 to 488, wherein the directionality of the surface texture follows the outline of a trapeze, a truncated circle, an oval, an ellipse, a pentagon, a hexagon, a heptagon, or an octagon.

Clause 496. The on-body device of any of clauses 475 to 488 or 492 to 495, wherein the directional surface texture is applied to the base of the housing in a concentric pattern.

Clause 497. The on-body device of clause 496, wherein a centroid or focus of the concentric pattern corresponds with the centroid of the shape of the periphery of the base.

Clause 498. The on-body device of clause 496 when directly or indirectly dependent on clause 326, wherein a centroid or focus of the concentric pattern corresponds with the location of the aperture in the housing.

Clause 499. The on-body device of any of clauses 475 to 488, wherein the directionality of the surface texture aligns with the shape of the periphery of the base.

Clause 500. The on-body device of any of clauses 475 to 488, wherein the surface texture is oriented so as to be continuously parallel with the peripheral edge of the base.

Clause 501. The on-body device of any of clauses 475 to 488, wherein the surface texture is oriented so as to be normal to with the peripheral edge of the base.

Clause 502. The on-body device of any of clauses 475 to 501 when directly or indirectly dependent on clause 330, wherein the surface texture is oriented directionally perpendicular to a long axis of the at least one channel.

Clause 503. The on-body device of any of any of clauses 475 to 502 when directly or indirectly dependent on clause 330, wherein the surface texture intersects the at least one channel.

Clause 504. The on-body device of any of any of clauses 475 to 502 when directly or indirectly dependent on clause 330, wherein the surface texture terminates in proximity with the channel.

Clause 505. The on-body device of clause 475 to 504, wherein the surface texture is a first surface texture and is in a first area of the base, and the base comprises a second surface texture in a second area of the base, wherein a directional orientation of the first surface texture is different from a directional orientation of the second surface texture.

Clause 506. The on-body device of clause 505 when directly or indirectly dependent on clause 330, wherein the first surface texture is directionally transverse to a first portion of the at least one channel having a long axis extending in a first direction, and the second surface texture is directionally transverse to a second portion of the channel having a long axis extending in a second direction different from the first direction.

Clause 507. The on-body device of clause 505 when directly or indirectly dependent on clause 332, wherein the first surface texture is directionally transverse to a first long axis of the one or more first channels, and the second surface texture is directionally transverse to a second long axis of the one or more second channels, wherein the first long axis and second long axis are non-parallel.

Clause 508. The on-body device of clause 505, wherein the first surface texture is directionally straight and the second surface texture is directionally curved.

Clause 509. The on-body device of any of clauses 470 to 508 when directly or indirectly dependent on clause 330, wherein the at least one channel has an average depth that is greater than the average depth of the surface texture.

Clause 510. The on-body device of clause 509, wherein the channel average depth is at least 10 μm greater than the surface texture average depth.

Clause 511. The on-body device of clause 509, wherein the channel average depth is at least 50 μm greater than the surface texture average depth.

Clause 512. The on-body device of clause 509, wherein the channel average depth is at least 100 μm greater than the surface texture average depth.

Clause 513. The on-body device of clause 509, wherein the channel average depth is at least 150 μm greater than the surface texture average depth.

Clause 514. The on-body device of any of clauses 470 to 513 when directly or indirectly dependent on clause 330, wherein at least a portion of the surface texture is oriented to facilitate movement of moisture or liquid towards the at least one channel.

Clause 515. The on-body device of any of clauses 470 to 514 when directly or indirectly dependent on clause 330, wherein at least one channel comprises a channel surface, and wherein at least a portion of the surface texture is on the channel surface.

Clause 516. The on-body device of clause 515, wherein the at least a portion of the surface texture on the channel surface is configured to facilitate movement of moisture along the channel.

Clause 517. The on-body device of any of clauses 470 to 516 when directly or indirectly dependent on clause 330, wherein a base surface adjacent the channel comprises directional surface texture oriented transverse to a long axis of the at least one channel.

Clause 518. The on-body device of any of clauses 470 to 517 when directly or indirectly dependent on clause 330, wherein the surface texture is configured to guide moisture to the at least one channel, and wherein the at least one channel is configured to guide the moisture to a periphery of the base and/or an end of the respective at least one channel.

Clause 519. The on-body device of any of clauses 470 to 518, wherein the surface texture has an arithmetic mean height roughness (Sa) of between 9 and 18 μm, or between 20 and 22 μm.

Clause 520. The on-body device of any of clauses 470 to 519, wherein the surface texture comprises, at least in part, non-directional surface texture.

Clause 521. The on-body device of clause 520, wherein the surface texture has an average roughness (Ra) of at least 0.35 μm.

Clause 522. The on-body device of clause 520, wherein the surface texture has an average roughness (Ra) of between 0.56 μm and 0.80 μm.

Clause 523. The on-body device of clause 520, wherein the surface texture has an average roughness (Ra) of between 1.12 μm and 1.60 μm.

Clause 524. The on-body device of any one of the preceding clauses, wherein the surface texture has an average roughness (Ra) of around 1.5 μm.

Clause 525. The on-body device of clause 520, wherein the surface texture has an average roughness (Ra) of between 0.35 μm and 2.24 μm.

Clause 526. The on-body device of any of clauses 520 to 525 when directly or indirectly dependent on clause 330, wherein the at least one channel has a first depth that is greater than an average depth of the surface texture.

Clause 527. The on-body device of clause 526, wherein the channel average depth is at least 10 μm greater than the surface texture average depth.

Clause 528. The on-body device of clause 526, wherein the channel average depth is at least 50 μm greater than the surface texture average depth.

Clause 529. The on-body device of clause 526, wherein the channel average depth is at least 100 μm greater than the surface texture average depth.

Clause 530. The on-body device of clause 526, wherein the channel average depth is at least 150 μm greater than the surface texture average depth.

Clause 531. The on-body device of any of clauses 470 to 530, wherein the surface texture is on an area of the base comprising adhesive.

Clause 532. The on-body device of any of clauses 470 to 531, wherein the surface texture comprises both non-directional and directional surface texture.

Clause 533. The on-body device of clause 324 or any preceding clause when directly or indirectly dependent thereon, further comprising a sensor.

Clause 534. The on-body device of clause 533, wherein the sensor comprises a sensor body configured to extend through the aperture in the housing and proximally from the base.

Clause 535. The on-body device of clause 533 or 534, wherein the sensor is an analyte sensor.

Clause 536. The on-body device of any of clauses 533 to 535, wherein the sensor is a wholly in vivo sensor or a wholly ex vivo sensor.

Clause 537. The on-body device of any of clauses 533 to 536, wherein the sensor is configured to be implanted in its entirety in the body of the user such that no part of the sensor protrudes from the body of the user during analyte monitoring.

Clause 538. The on-body device of any of clauses 533 to 537, wherein the electronics are configured to receive the signal wirelessly from the sensor while the sensor is implanted within the body of the user.

Clause 539. The on-body device of any of clauses 533 to 536, wherein the sensor comprises a first portion coupled to the electronics and a second portion configured to be positioned in the body of the user for monitoring the level of the analyte of the user.

Clause 540. The on-body device of any of clauses 533 to 539, wherein the sensor is configured to measure an analyte level.

Clause 541. The on-body device of clause 540, wherein the analyte is glucose.

Clause 542. The on-body device of clause 540, wherein the analyte is one or more ketones.

Clause 543. The on-body device of clause 540, wherein the analyte is lactate.

Clause 544. The on-body device of clause 540, wherein the analyte is oxygen.

Clause 545. The on-body device of clause 540, wherein the analyte is ammonium.

Clause 546. The on-body device of clause 540, wherein the analyte is asparagine.

Clause 547. The on-body device of clause 540, wherein the analyte is aspartate.

Clause 548. The on-body device of clause 540, wherein the analyte is cholesterol.

Clause 549. The on-body device of clause 540, wherein the analyte is choline.

Clause 550. The on-body device of clause 540, wherein the analyte is creatinine.

Clause 551. The on-body device of clause 540, wherein the analyte is a diaphorase inhibitor.

Clause 552. The on-body device of clause 540, wherein the analyte is ethanol.

Clause 553. The on-body device of clause 540, wherein the analyte is glutamate.

Clause 554. The on-body device of clause 540, wherein the analyte is glycerol.

Clause 555. The on-body device of clause 540, wherein the analyte is lactose.

Clause 556. The on-body device of clause 540, wherein the analyte is phosphate.

Clause 557. The on-body device of clause 540, wherein the analyte is potassium.

Clause 558. The on-body device of clause 540, wherein the analyte is pyruvate.

Clause 559. The on-body device of clause 540, wherein the analyte is uric acid.

Clause 560. The on-body device of clause 540, wherein the analyte is a first analyte, and the sensor is further configured to measure a level of a second analyte of the user.

Clause 561. The on-body device of clause 560, wherein the first analyte is glucose and the second analyte is a ketone.

Clause 562. The on-body device of clause 560, wherein the first analyte is glucose and the second analyte is lactate.

Clause 563. The on-body device of clause 560, wherein the first analyte is lactate and the second analyte is a ketone.

Clause 564. The on-body device of clause 560, wherein the first and second analytes are selected from the list consisting of: ammonium, asparagine, aspartate, cholesterol, choline, creatinine, diaphorase inhibitors, ethanol, glucose, glutamate, glycerol, one or more ketones, lactate, lactose, oxygen, phosphate, potassium, pyruvate, and uric acid.

Clause 565. The on-body device of any of clauses 533 to 537, wherein the analyte sensor is a glucose sensor comprising: a proximal portion configured to electrically couple with the electronics; a distal portion having a glucose-responsive enzyme, wherein the distal portion is configured to be positioned in a bodily fluid of the user's body and to sense glucose in the user's bodily fluid in a subcutaneous layer.

Clause 566. The on-body device of any clause 325 or any preceding clause when directly or indirectly dependent thereon, further comprising a moisture permeable material attached to at least a portion of the base.

Clause 567. The on-body device of clause 566, wherein the moisture permeable element is arranged on the entirety of the first surface of the base.

Clause 568. The on-body device of clause 566, wherein the moisture permeable element comprises a plurality of portions on the base.

Clause 569. The on-body device of clause 566, wherein the moisture permeable element extends to a periphery of the base.

Clause 570. The on-body device of any of clauses 566 to 569, wherein the moisture permeable element is arranged within a recess in the base.

Clause 571. The on-body device of any of clauses 566 to 570, wherein the moisture permeable material is flush with the first surface of the base.

Clause 572. The on-body device of any of clauses 566 to 571, wherein the base comprises a plurality of surfaces configured to face towards the skin surface of the user, and wherein the moisture permeable material is located across the plurality of surfaces.

Clause 573. The on-body device of any of clauses 566 to 571, wherein the base comprises a plurality of surfaces configured to face towards the skin surface of the user, and wherein the moisture permeable material is located across at least a portion of one of the plurality of surfaces.

Clause 574. The on-body device of any of clauses 566 to 573 when directly or indirectly dependent on clause 7, further comprising a moisture permeable material within the at least one channel.

Clause 575. The on-body device of clause 574, wherein the moisture permeable material extends along an entire length of the at least one channel.

Clause 576. The on-body device of clauses 574 or 575, wherein the moisture permeable material is located within the channel and across at least a portion of the first surface of the base.

Clause 577. The on-body device of any of clauses 574 to 576, wherein the at least one channel has a channel bottom located distal to a channel top, and wherein a width at the channel bottom is greater than a width at the channel top to secure the moisture permeable material within the channel.

Clause 578. The on-body device of any of clauses 566 to 577, wherein the moisture permeable material comprises silicone.

Clause 579. The on-body device of any of clauses 566 to 577, wherein the moisture permeable material comprises poly(methyl methacrylate).

Clause 580. The on-body device of any of clauses 566 to 577, wherein the moisture permeable material comprises a moisture permeable fibrous material.

Clause 581. The on-body device of any of clauses 566 to 577, wherein the moisture permeable material comprises polyurethane (PU).

Clause 582. The on-body device of any of clauses 566 to 577, wherein the moisture permeable material comprises polytetrafluoroethylene (PTFE).

Clause 583. The on-body device of any of clauses 566 to 577, wherein the moisture permeable material comprises polyethylene (PE).

Clause 584. The on-body device of any of clauses 566 to 577, wherein the moisture permeable material comprises nylon (polyamide).

Clause 585. The on-body device of clause 325 or any preceding clause when directly or indirectly dependent thereon, further comprising an adhesive patch attached to the housing and configured to attach the housing to skin of the user.

Clause 586. The on-body device of clause 585, wherein the adhesive patch comprises an adhesive on a first side for coupling the adhesive patch to the base.

Clause 587. The on-body device of clause 585 or 586, wherein the adhesive patch comprises an adhesive on a second side for adhering the adhesive patch to the skin of the user.

Clause 588. The on-body device of clause 586 and 587 when directly or indirectly dependent on any of clauses 566-584, wherein the adhesive patch is attached to the housing and is in contact with the moisture permeable material, and wherein the moisture permeable material is configured to allow water molecules to diffuse from the adhesive patch and through the moisture permeable material.

Clause 589. The on-body device of any of clauses 585 to 588 when directly or indirectly dependent on clause 330, wherein the adhesive patch comprises one or more openings in one or more positions corresponding to the at least one channel.

Clause 590. The on-body device of any preceding clause, wherein the housing is configured to be coupled to a body of the user.

Clause 591. The on-body device of any preceding clause, wherein the housing encompasses the electronics.

Clause 592. The on-body device of any preceding clause, wherein the housing is a first housing, and the on-body device comprises a second housing that encompasses the electronics.

Clause 593. The on-body device of clause 592, wherein the second housing is configured to couple with the first housing.

Clause 594. The on-body device of clause 592 or 593, wherein the first housing is configured to hold the second housing and the electronics.

Clause 595. The on-body device of any of clauses 592 to 594, wherein the first housing is configured as a receptacle for the second housing.

Clause 596. The on-body device of clause 325 or any preceding clause when directly or indirectly dependent thereon, wherein the area adjacent the housing comprises at least one of: space between the base and an adhesive; adhesive; a flexible patch; and a skin surface.

Clause 597. The on-body device of any preceding clause, wherein the on-body device comprises an injection molded housing.

Clause 598. The on-body device of any of clauses 324 to 596, wherein the on-body device comprises a one-shot molded or over-molded housing.

Clause 599. The on-body device of any of clauses 324 to 596, wherein the on-body device comprises a housing comprising a first part and a second part assembled together with sensor electronics arranged between.

Clause 600. The on-body device of any of clauses 324 to 596, wherein the on-body device comprises a housing with a recess configured to receive a sensor connector, wherein the recess is arranged in a top of the housing or in a bottom of the housing.

Clause 601. The on-body device of any of clauses 324 to 596, wherein the on-body device comprises a housing with a recess configured to receive a sensor connector, wherein the recess is arranged in a top of the housing or in a bottom of the housing.

Clause 602. The on-body device of any of clauses 597 to 601, wherein the shape of the peripheral edge of the base of the housing comprises one or more indentations and/or one or more protrusions or lobes.

Clause 603. The on-body device of clause 326 or any preceding clause when directly or indirectly dependent thereon, wherein the aperture is located at a centroid of the shape of the peripheral edge of the base of the housing.

Clause 604. The on-body device of clause 326 or any preceding clause when directly or indirectly dependent thereon, wherein the aperture is offset from a centroid of the shape of the peripheral edge of the base of the housing.

Clause 605. The on-body device of clause 604, wherein the offset is along an axis of symmetry of the shape.

Clause 606. The on-body device of clause 604, wherein the offset is along an axis that is perpendicular to an axis of symmetry of the shape and in the plane of the base.

Clause 607. The on-body device of clause 604, wherein the offset is along both an axis of symmetry of the shape and an axis that is perpendicular to the axis of symmetry of the shape.

Clause 608. The on-body device of clause 604, wherein the aperture is located in a corner of the shape and spaced apart from the periphery of the base.

Clause 609. The on-body device of any preceding clause, wherein the electronics comprise a processor, memory, a power supply, and wireless communication circuitry configured to wirelessly communicate data indicative of a glucose level.

Clause 610. The on-body device of any preceding clause, wherein the electronics is configured to be disposed after a single use.

Clause 611. An insertion system, comprising: the on-body device of any preceding clause; and an applicator configured to position the on-body device on the skin of the user.

Clause 612. The insertion system of clause 611 when directly or indirectly dependent on clause 533, wherein the applicator is configured to position the sensor in the body of the user.

Clause 613. The insertion system of clause 611 or 612 when directly or indirectly dependent on clause 533, wherein the applicator is configured to advance the on-body device along a linear path toward the skin surface of the user, such that a sharp carrying the sensor is configured to pierce the skin of the user at an angle substantially perpendicular to the skin of the user.

Clause 614. An analyte monitoring system, comprising: the on-body device according to any of clauses 324 to 608; and a receiver unit configured to communicate with the on-body device for receiving data associated with the analyte level of the user.

Clause 615. The analyte monitoring system of clause 614, wherein the receiver unit is configured to receive the data indicative of the monitored level of the analyte according to a Bluetooth or Bluetooth Low Energy (BLE) protocol.

Clause 616. The analyte monitoring system of clause 614 or 615, wherein the receiver unit is configured to receive the data indicative of the monitored level of the analyte according to a Near Field Communication (NFC) protocol.

Clause 617. The analyte monitoring system of any of clauses 614 to 616, further comprising a plurality of receiver units, wherein the plurality of receiver units comprises the receiver unit, wherein the plurality of receiver units are configured to receive data indicative of the monitored level of the analyte from the on-body device, wherein the on-body device is configured to authenticate the plurality of receiver units, and wherein the plurality of receiver units comprise a smart phone and a smart watch.

Clause 618. A system comprising: the insertion system of clause 611 or 612; and the analyte monitoring of any of clauses 614 to 617.

Clause 619. A method of monitoring an analyte of a user, comprising: applying an on-body device to a body of the user; and monitoring a level of the analyte with the on-body device, wherein the on-body device is configured in accordance with any of clauses 1 to 608.

Clause 620. The method of clause 619, wherein monitoring the level of the analyte with the on-body device comprises monitoring the level of the analyte with the on-body device for a wear duration without removing the on-body device from the body of the user.

Clause 621. The method of clause 620, wherein the wear duration is 7 days or greater.

Clause 622. The method of clause 620, wherein the wear duration is 14 days or greater.

Clause 623. The method of clause 620, wherein the wear duration is 21 days or greater.

Clause 624. The method of clause 620, wherein the wear duration is 30 days or greater.

Clause 625. The method of clause 620, wherein the wear duration is 45 days or greater.

Clause 626. The method of clause 620, wherein the wear duration is between 7 days and 60 days.

Clause 627. The method of any of clauses 619 to 626, further comprising transmitting, from the on-body device to a receiver unit, data indicative of the monitored level of the analyte.

Clause 628. The method of any of clauses 619 to 626, wherein the analyte is a first analyte, the method further comprising monitoring a level of a second analyte with the on-body device.

Clause 629. The method of clause 628, further comprising transmitting, from the on-body device to a receiver unit, data indicative of the monitored level of the first analyte and the monitored level of the second analyte.

Clause 630. The method of clause 627 to 629, wherein the data indicative of the monitored level of the analyte is transmitted according to a Bluetooth or Bluetooth Low Energy (BLE) protocol.

Clause 631. The method of clause 627 to 629, wherein the data indicative of the monitored level of the analyte is transmitted according to a Near Field Communication (NFC) protocol.

Clause 632. The method of clause 627 or any of clauses 629 to 631, wherein the receiver unit is a smart phone.

Clause 633. The method of clause 627 or any of clauses 629 to 631, wherein the receiver unit is a dedicated receiver unit.

Clause 634. The method of clause 627 or any of clauses 629 to 631, wherein the receiver unit is a smart watch.

Clause 635. The method of any of clauses 619 to 626, further comprising transmitting, from the on-body device to a plurality of receiver units, data indicative of the monitored level of the analyte, wherein the plurality of receiver units are each in an authenticated state at the on-body device, and wherein the plurality of receiver units comprise a smart phone and a smart watch.

Clause 636. The method of any of clauses 619 to 635, wherein applying the on-body device to the body of the user comprises applying the on-body device in a position over a wholly in vivo sensor.

Clause 637. The method of any of clauses 619 to 636, wherein applying the on-body device to the body of the user comprises using an applicator to apply the on-body device to the skin of the user and/or to implant a sensor.

Clause 638. The method of clause 636, further comprising wirelessly transmitting data indicative of the level of the analyte from the wholly in vivo sensor to the on-body device.

Clause 639. The method of clause 638, wherein the data indicative of the monitored level of the analyte is transmitted from the wholly in vivo sensor according to a Bluetooth or Bluetooth Low Energy (BLE) protocol.

Clause 640. The method of clause 638, wherein the data indicative of the monitored level of the analyte is transmitted from the wholly in vivo sensor according to a Near Field Communication (NFC) protocol.

Clause 641. A method of manufacturing an on-body device for monitoring an analyte level of a user, comprising: providing a housing; and providing electronics within the housing, wherein the electronics are configured to receive a signal from a sensor, the signal indicative of a level of an analyte of a user, wherein the housing is adapted to permit egress of moisture from an area adjacent the housing.

Clause 642. The method of clause 641, further comprising providing an aperture in a first region in a base of the housing.

Clause 643. The method of clause 642, further comprising providing at least one channel along a first surface of the base of the housing, wherein the at least one channel is configured to permit egress of moisture from an area adjacent the first surface.

Clause 644. The method of clause 643, wherein providing the at least one channel comprises: providing one or more first channels, wherein each of the one or more first channels comprise a first end arranged at a periphery of the base, and a second end arranged at the first region of the base; providing one or more second channels, wherein each of the one or more second channels comprises a first end arranged at the periphery of the base and a second end arranged at the periphery of the base.

Clause 645. The method of clause 644, wherein providing the one or more second channels comprises providing one or more curved channels that intersect the one or more first channels.

Clause 646. The method of any of clauses 641 to 645, further comprising applying a directional surface texture to a base of the housing.

Clause 647. The method of clause 646, wherein the applying of the directional surface texture is by a laser process, electrical discharge machining, or wire brush process.

Clause 648. The method of any of clauses 641 to 647, further comprising applying a non-directional surface texture to a base.

Clause 649. The method of clause 648, wherein the applying of the non-directional surface texture comprises is by a bead blasting process.

Clause 650. A method of manufacturing the on-body device according to any of clauses 1 to 608, comprising: providing the housing; and providing the electronics within the housing, wherein the electronics are configured to receive a signal from a sensor, the signal indicative of a level of an analyte of a user, wherein the housing is adapted to permit egress of moisture from an area adjacent the housing.

Clause 651. A kit of parts, comprising: the on-body device of any of clauses 324 to 610; and an applicator configured to position the on-body device on the skin of the user.

Clause 652. The on-body device of any of clauses 324 to 610 in combination with the features of the on-body device of any of clauses 1 to 154 or 178 to 288 or 322 to 323.

Clause 653. The on-body device of any of clauses 1 to 154 or 178 to 288 or 322 to 323 in combination with the features of the on-body device of any of clauses 324 to 610.

Clause 654. An adhesive patch for securing an on-body device to a user's body, the adhesive patch comprising: a plurality of patch portions, each of the plurality of patch portions defining a lower surface configured to face a user's body in use and an upper surface configured to face away from the user's body in use, wherein the plurality of patch portions comprises: a primary patch portion having an outer periphery, and one or more removable patch portions, wherein each lower surface of the one or more removable patch portions comprises an adhesive layer for adhering to the user's body, wherein at least one of the one or more removable patch portions is releasably coupled to the primary patch portion.

Clause 655. The adhesive patch of clause 654, wherein the lower surface of the primary patch portion comprises an adhesive layer.

Clause 656. The adhesive patch of clause 655, wherein the adhesive layer comprises a pressure sensitive adhesive, optionally wherein the pressure sensitive adhesive is an acrylic based pressure sensitive adhesive.

Clause 657. The adhesive patch of clause 655 or 656, wherein the thickness of the adhesive layer of the lower surface of the primary patch portion is between 50 μm to 125 μm or 75 μm to 100 μm Clause 658. The adhesive patch of any of clause 654 to 657, wherein the patch comprises one or more release liners covering the adhesive layer of the primary patch portion.

Clause 659. The adhesive patch of any of clause 654 to 658, wherein the patch comprises one or more release liners covering the respective adhesive layers of the one or more removable patch portions.

Clause 660. The adhesive patch of any of clause 654 to 659, wherein the patch comprises one or more release liners covering each of the adhesive layers of the plurality of patch portions.

Clause 661. The adhesive patch of clauses 658 to 660, wherein the one or more release liners are contiguous and/or form a continuous surface.

Clause 662. The adhesive patch of any of clauses 658 to 661, wherein the release liner comprises silicone coated paper or silicone coated polyester film.

Clause 663. The adhesive patch of any of clause 654 to 662, wherein at least one, or all, of the respective adhesive layers of the one or more removable patch portions comprises a pressure sensitive adhesive, optionally an acrylic based pressure sensitive adhesive.

Clause 664. The adhesive patch of any of clause 654 to 663, wherein the thickness of at least one, or each, of the adhesive layers of the removable patch portions is between 50 μm to 125 μm or 75 μm to 100 μm.

Clause 665. The adhesive patch of any of clause 654 to 664, wherein the upper surface of the primary patch portion comprises a device adhesive for securing an on-body device to the adhesive patch, optionally wherein the device adhesive comprises a pressure sensitive adhesive, optionally an acrylic based pressure sensitive adhesive.

Clause 666. The adhesive patch of any of clause 654 to 665, wherein the primary patch portion and/or the one or more removable patch portions comprise one or more backing layers defining the respective upper surface and the lower surface.

Clause 667. The adhesive patch of clause 666, wherein at least one, or each, of the one or more backing layers has a thickness between 10 μm and 300 μm, between 15 μm and 275 μm, between 40 μm and 200 μm, or between 50 μm and 150 μm.

Clause 668. The adhesive patch of any of clause 654 to 667, wherein the primary patch portion comprises an aperture through which a cannula of an on-body device may pass.

Clause 669. The adhesive patch of any of clause 654 to 668, wherein the primary patch portion comprises an aperture sized to at least partially pass over the top of an on-body device when the on-body device is adhered to the user's body.

Clause 670. The adhesive patch of any of clause 654 to 669, wherein the primary patch portion comprises a substantially rigid locating feature configured to mate with the on-body device.

Clause 671. The adhesive patch of clause 670, wherein the substantially rigid locating feature forms an aperture sized to at least partially pass over the top of an on-body device when the on-body device is adhered to the user's body.

Clause 672. The adhesive patch of any of clauses 654 to 671, wherein the on-body device comprises an injection molded housing.

Clause 673. The adhesive patch of any of clauses 654 to 671, wherein the on-body device comprises a one-shot molded or over-molded housing.

Clause 674. The adhesive patch of any of clauses 654 to 671, wherein the on-body device comprises a housing comprising a first part and a second part assembled together with sensor electronics arranged between.

Clause 675. The adhesive patch of any of clauses 654 to 671, wherein the on-body device comprises a housing with a recess configured to receive a sensor connector, wherein the recess is arranged in a top of the housing or in a bottom of the housing.

Clause 676. The adhesive patch of any of clause 654 to 675, wherein at least one, or all, of the plurality of patch portions are formed from a non-woven material, optionally wherein the non-woven material is a polyurethane or polyester spun-lace material.

Clause 677. The adhesive patch of any of clause 654 to 676, wherein the one or more removable patch portions is a plurality of removable patch portions, wherein each of the plurality of removable patch portions is releasably coupled to another one of the patch portions.

Clause 678. The adhesive patch of clause 677, wherein the plurality of removable patch portions comprises one or more first removable patch portions, wherein each of the one or more first removable patch portions is releasably coupled to the primary patch portion

Clause 679. The adhesive patch of any one of clauses 677 or 678, wherein at least one of the one or more first removable patch portions is releasably coupled to the primary patch portion and to another of the one or more first removable patch portions.

Clause 680. The adhesive patch of any one of clauses 677 to 679 wherein at least one of the one or more first removable patch portions is releasably coupled to the primary patch portion and to another two of the one or more first removable patch portions.

Clause 681. The adhesive patch of any one of clauses 677 to 680 wherein the one or more first removable patch portions comprises or consists of two removable patch portions.

Clause 682. The adhesive patch of any one of clause 677 to 680 wherein the one or more first removable patch portions comprises or consists of three removable patch portions.

Clause 683. The adhesive patch of any one of clauses 677 to 680, wherein the one or more first removable patch portions comprises or consists of four removable patch portions.

Clause 684. The adhesive patch of any one of clauses 677 to 680, wherein the one or more first removable patch portions comprises or consists of five removable patch portions.

Clause 685. The adhesive patch of any one of clauses 677 to 680, wherein the one or more first removable patch portions comprises or consists of between six and ten removable patch portions.

Clause 686. The adhesive patch of any one of clauses 677 to 685, wherein at least one, or each, of the one or more first removable patch portions has a polygonal shape, optionally wherein the polygonal shape is a rectangular or trapezoidal shape.

Clause 687. The adhesive patch of any one of clauses 677 to 686 wherein at least one, or each, of the one or more first removable patch portions has a crescent shape.

Clause 688. The adhesive patch of any one of clauses 677 to 685, wherein at least one, or each, of the one or more first removable patch portions has a one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Clause 689. The adhesive patch of any one of clauses 677 to 687, wherein each of the one or more first removable patch portions is the same shape and/or is the same size.

Clause 690. The adhesive patch of any one of clauses 678 to 689, wherein the plurality of removable patch portions comprises one or more second removable patch portions, wherein each of the one or more second removable patch portions is not releasably coupled to the primary patch portion but is releasably coupled to at least one of the one or more first removable patch portions.

Clause 691. The adhesive patch of clause 690, wherein at least one of the one or more second removable patch portions is releasably coupled to at least another of the one or more second removable patch portions.

Clause 692. The adhesive patch of clause 690 or 691, wherein at least one of the one or more second removable patch portions is releasably coupled to at least two of the one or more second removable patch portions.

Clause 693. The adhesive patch of any one of clauses 690 to 692, wherein each of the one or more second removable patch portions is releasably coupled to at least two of the one or more second removable patch portions.

Clause 694. The adhesive patch of any one of clauses 690 to 693, wherein the one or more second removable patch portions comprises or consists of two removable patch portions.

Clause 695. The adhesive patch of any one of clauses 690 to 693, wherein the one or more second removable patch portions comprises or consists of three removable patch portions.

Clause 696. The adhesive patch of any one of clauses 690 to 693, wherein the one or more second removable patch portions comprises or consists of four removable patch portions.

Clause 697. The adhesive patch of any one of clauses 690 to 693, wherein the one or more second removable patch portions comprises or consists of five removable patch portions.

Clause 698. The adhesive patch of any one of clauses 690 to 693, wherein the one or more second removable patch portions comprises or consists of between six and ten removable patch portions.

Clause 699. The adhesive patch of any one of clauses 690 to 698 wherein at least one, or each, of the one or more second removable patch portions has a polygonal shape, optionally wherein the polygonal shape is a rectangular or trapezoidal shape.

Clause 700. The adhesive patch of any one of clauses 690 to 699 wherein at least one, or each, of the one or more second removable patch portions has a crescent shape.

Clause 701. The adhesive patch of any one of clauses 690 to 698, wherein at least one, or each of the one or more second removable patch portions has a one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Clause 702. The adhesive patch of any one of clauses 690 to 701, wherein each of the one or more second removable patch portions is the same shape and/or is the same size.

Clause 703. The adhesive patch of any one of clauses 690 to 702, where at least one, or all, of the one or more first removable patch portions is smaller than at least one, or all, of the one or more second removable patch portions.

Clause 704. The adhesive patch of any one of clauses 690 to 703, where at least one, or all, of the one or more first removable patch portions is larger than at least one, or all, of the one or more second removable patch portions.

Clause 705. The adhesive patch of any one of clauses 678 to 704, wherein the plurality of removable patch portions comprises one or more third removable patch portions, wherein each of the one or more third removable patch portions is not releasably coupled to the primary patch portion or to the one or more first removable patch portions.

Clause 706. The adhesive patch of clause 705, wherein each of the one or more third removable patch portions is indirectly coupled to the primary patch portion.

Clause 707. The adhesive patch of clause 705 or 706, wherein at least one of the one or more third removable patch portions is releasably coupled to at least one of the one or more second removable patch portions.

Clause 708. The adhesive patch of any one of clauses 705 to 707, wherein at least one, or each, of the one or more third removable patch portions has a polygonal shape, optionally wherein the polygonal shape is a rectangular or trapezoidal shape.

Clause 709. The adhesive patch of any one of clauses 705 to 708, wherein at least one, or each, of the one or more third removable patch portions has a crescent shape.

Clause 710. The adhesive patch of any one of clauses 705 to 707 wherein at least one, or each, of the one or more third removable patch portions has a one or more of: a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Clause 711. The adhesive patch of any one of clauses 705 to 709, wherein each of the one or more third removable patch portions has the same shape, has the same size, and/or has the same size and shape.

Clause 712. The adhesive patch of any one of clauses 705 to 711, wherein at least one, or all, of the one or more first removable patch portions is smaller than at least one, or all, of the one or more third removable patch portions.

Clause 713. The adhesive patch of any one of clauses 705 to 712, wherein at least one, or all, of the one or more first removable patch portions is larger than at least one, or all, of the one or more third removable patch portions.

Clause 714. The adhesive patch of any one of clauses 705 to 713, when directly or indirectly dependent on clause 690, wherein at least one, or all, of the one or more second removable patch portions is smaller than at least one, or all, of the one or more third removable patch portions.

Clause 715. The adhesive patch of any one of clauses 705 to 714, when directly or indirectly dependent on clause 690, wherein at least one, or all, of the one or more second removable patch portions is larger than at least one, or all, of the one or more third removable patch portions.

Clause 716. The adhesive patch of any of clause 654 to 715, wherein the plurality of patch portions extend in or along substantially the same plane or curved surface.

Clause 717. The adhesive patch of any of clause 654 to 716, wherein the primary patch portion is entirely encompassed by the plurality of removable patch portions.

Clause 718. The adhesive patch of any one of clauses 678 to 717, wherein each of the first or more removable patch portions is releasably coupled to the outer periphery of the primary patch portion.

Clause 719. The adhesive patch of any one of clauses 678 to 718, wherein the primary patch portion and the one or more first removable patch portions extend in or along substantially the same plane or curved surface.

Clause 720. The adhesive patch of any one of clauses 678 to 719, wherein the primary patch portion is entirely encompassed by the one or more first removable patch portions.

Clause 721. The adhesive patch of any one of clauses 678 to 720, wherein at least 30%, at least 90%, or about 100% of the length of the outer periphery of the primary patch portion is releasably connected to the one or more first removable patch portions.

Clause 722. The adhesive patch of any one of clauses 678 to 721, wherein the one or more first removable patch portions are uniformly distributed about the outer periphery of the primary patch portion.

Clause 723. The adhesive patch of any one of clauses 678 to 722, wherein the one or more first removable patch portions are located with a rotational symmetry of order two or more with respect to a point within the primary patch portion.

Clause 724. The adhesive patch of clause 723 wherein the order of the rotational symmetry is equal to the number of first one or more removable patch portions.

Clause 725. The adhesive patch of any one of clauses 690 to 724, wherein the primary patch portion is entirely encompassed by the one or more second removable patch portions.

Clause 726. The adhesive patch of any one of clauses 690 to 725, wherein the one or more second removable patch portions are uniformly distributed about outer periphery of the primary patch portion.

Clause 727. The adhesive patch of any one of clauses 690 to 726, wherein the one or more second removable patch portions are located with a rotational symmetry of order two or more with respect to a point within the primary patch portion.

Clause 728. The adhesive patch of clause 727 wherein the order of the rotational symmetry is equal to the number of the one or more second removable patch portions.

Clause 729. The adhesive patch of any one of clauses 705 to 728 wherein the primary patch portion is entirely encompassed by the one or more third removable patch portions.

Clause 730. The adhesive patch of any one of clauses 690 to 728 wherein the primary patch portion is entirely encompassed by a combination of the one or more first removable patch portions and the one or more second removable patch portions.

Clause 731. The adhesive patch of any one of clauses 705 to 730, wherein the primary patch portion is entirely encompassed by a combination of the one or more first removable patch portions and the one or more third removable patch portions.

Clause 732. The adhesive patch of any one of clauses 705 to 731 when directly or indirectly dependent on clauses 678 and 690, wherein the primary patch portion is entirely encompassed by a combination of the one or more first removable patch portions, the one or more second removable patch portions and the one or more third removable patch portions.

Clause 733. The adhesive patch of any one of clauses 678 to 732, wherein the primary patch portion is not entirely encompassed by the one or more first removable patch portions.

Clause 734. The adhesive patch of any one of clauses 690 to 733, wherein the primary patch portion is not entirely encompassed by the one or more second removable patch portions.

Clause 735. The adhesive patch of any one of clauses 705 to 734 wherein the primary patch portion is not entirely encompassed by the one or more third removable patch portions.

Clause 736. The adhesive patch of any of clause 654 to 735, wherein the plurality of removable patch portions form one or more concentric rings around the primary patch portion, optionally wherein the plurality of removable patch portions form three, four, or five concentric rings around the primary patch portion.

Clause 737. The adhesive patch of any of clause 654 to 736, wherein each of the plurality of removable patch portions is releasably connected to adjacent ones of the primary patch portion and plurality of removable patch portions.

Clause 738. The adhesive patch of any one of clauses 654 to 711, when directly or indirectly dependent on clause 678, wherein the one or more first removable patch portions are releasably connected to the lower surface of the primary patch portion.

Clause 739. The adhesive patch of any one of clauses 654 to 711 or 738, wherein the lower surface of the primary patch portion comprises an adhesive layer, and the one or more first removable patch portions are releasably connected to the adhesive layer of the primary patch portion.

Clause 740. The adhesive patch of clause 738 or 739, wherein the upper surfaces of the one or more first removable patch portions comprise a release liner configured to adhere to the adhesive layer of the primary patch portion less strongly than the user's body would adhere to the adhesive layer.

Clause 741. The adhesive patch of any one of clauses 738 to 740, wherein the primary patch portion extends in or along a first plane or curved surface and the one or more first removable patch portions extend in or along a second plane which is offset from but substantially parallel to the first plane.

Clause 742. The adhesive patch of any one of clauses 738 to 741, wherein the lower surface of the primary patch portion is at least 30% covered, at least 90% covered, or entirely covered, by the one or more first removable patch portions.

Clause 743. The adhesive patch of any one of clauses 738 to 742, wherein the one or more first removable patch portions form a grid pattern.

Clause 744. The adhesive patch of any one of clauses 738 to 743 wherein at least one of the one or more first removable patch portions is not coupled to any other one of the one or more first removable patch portions or to any other one of the plurality of removable patch portions.

Clause 745. The adhesive patch of any one of clauses 738 to 744 wherein the one or more first removable patch portions define a continuous or discontinuous outer periphery.

Clause 746. The adhesive patch of clause 745 wherein at least part, or all of, of the outer periphery of the one or more first removable patch portions is more outward than at least part of, or all of, the outer periphery of the primary patch portion.

Clause 747. The adhesive patch of any one of clauses 745 or 746, wherein at least part of, or all of, of the outer periphery of the one or more first removable patch portions is coincident with the outer periphery of the primary patch portion.

Clause 748. The adhesive patch of any one of clause 745 to 747, wherein each of the one or more first removable patch portions extends to the outer periphery.

Clause 749. The adhesive patch of any one of clauses 745 to 748 wherein the outer periphery of the one or more first removable patch portions encompasses a greater area than the outer periphery of the primary patch portion.

Clause 750. The adhesive patch of any one of clauses 745 to 749 wherein the outer periphery of the one or more first removable patch portions encompasses a smaller area than the outer periphery of the primary patch portion.

Clause 751. The adhesive patch of any of clauses 654 to 750, wherein at least one of the one or more removable patch portions defines a path-following removable patch portion, wherein the path-following removable patch portion is configured to follow a path as it is progressively removed from the adhesive patch.

Clause 752. The adhesive patch of clause 751, wherein the adhesive patch and/or the path-following removable patch portion comprises an indica or marker indicating a predetermined point from which the path-following removable patch portion should be removed so that it follows the path.

Clause 753. The adhesive patch of clause 751 or 752, wherein the path is concertina shaped.

Clause 754. The adhesive patch of any of clauses 751 to 753, wherein the path doubles back on itself.

Clause 755. The adhesive patch of clause 751 or 752, wherein the path is spiral shaped, optionally wherein spiral is a square or rectangular spiral or is a curved spiral.

Clause 756. The adhesive patch of any one of clauses 751 to 755, wherein the path-following removable portion comprises one or more releasable self-couplings that removably couple the path-following removable patch portion to itself, wherein the one or more releasable self-couplings are configured such that the path-following removable patch portion preferentially follows the path as it is removed from the patch.

Clause 757. The adhesive patch of any one of clauses 751 to 756, wherein the path-following removable patch portion comprises one or more releasable self-couplings that removably couple the path-following removable patch portion to itself, wherein the one or more releasable self-couplings are configured to break as the path-following removable patch portion is removed along the path.

Clause 758. The adhesive patch of any one of clauses 756 to 757, wherein the one or more releasable self-couplings comprises one or more partial releasable self-couplings which extend only partially into the path-following removable portion such that the one or more partial releasable self-couplings break as the path-following removable patch portion is removed.

Clause 759. The adhesive patch of clause 758, wherein the one or more partial releasable self-couplings extend from a respective edge of the path-following removable portion to a respective point within the path-following removable patch portion.

Clause 760. The adhesive patch of clause 758 or 759, wherein at least one of the one or more partial releasable self-couplings is substantially straight.

Clause 761. The adhesive patch of any of clause 758 to 760, wherein at least one of the one or more partial releasable self-couplings is substantially curved.

Clause 762. The adhesive patch of any of clause 758 to 761, wherein at least one of the one or more partial releasable self-couplings follows a spiral shape.

Clause 763. The adhesive patch of any one of clauses 758 to 762, wherein the one or more releasable self-couplings comprise at least two partial releasable self-couplings that are substantially parallel and offset relative to each other.

Clause 764. The adhesive patch of any one of clauses 758 to 763, wherein the one or more releasable self-couplings comprise at least two partial releasable self-couplings which extend from opposite edges of the path-following removable patch portion.

Clause 765. The adhesive patch of clause 763 or 764, wherein the at least two partial releasable self-couplings overlap without contact.

Clause 766. The adhesive patch of any one of clauses 751 to 765, wherein the path-following removable patch portion is bounded by an outer periphery and an inner periphery, and wherein the one or more releasable self-couplings comprises a guiding releasable self-coupling that extends from the outer periphery to the inner periphery of the path-following removable patch portion.

Clause 767. The adhesive patch of clause 766, wherein the guiding releasable self-coupling is substantially straight.

Clause 768. The adhesive patch of clause 766, wherein the guiding releasable self-coupling is substantially curved.

Clause 769. The adhesive patch of any one of clauses 766 to 768, wherein the guiding releasable self-coupling follows a spiral.

Clause 770. The adhesive patch of any one of clauses 766 to 769, wherein the guiding releasable self-coupling doubles back on itself and/or is concertinaed.

Clause 771. The adhesive patch of any one of clauses 766 to 770, wherein the inner periphery of the path-following removable portion is releasably coupled to the outer periphery of the primary patch portion.

Clause 772. The adhesive patch of any one of clauses 766 to 771 wherein the inner periphery of the path-following removable portion is releasably coupled to the outer periphery of the primary patch portion by a releasable coupling extending along the outer periphery of the primary patch portion.

Clause 773. The adhesive patch of any one of clauses 766 to 772, wherein the path-following removable portion is releasably connected to the outer periphery of the primary patch portion.

Clause 774. The adhesive patch of any one of clauses 751 to 770, wherein the path-following removable portion is releasably connected to the lower surface of the primary patch portion.

Clause 775. The adhesive patch of any one of clauses 751 to 774, wherein the path-following removable patch portion is defined by at least two removable patch portions, wherein the at least two removable patch portions are arranged as a lengthways sequence along a length of the path.

Clause 776. The adhesive patch of any one of clauses 751 to 775 wherein the path-following removable patch portion is defined by at least two removable patch portions, wherein the at least two removable patch portions are delineated by at least one releasable coupling extending completely or partially across the path.

Clause 777. The adhesive patch of clause 776, wherein the at least releasable coupling extending completely or partially across the path extends substantially orthogonal to direction of the path at that location.

Clause 778. The adhesive patch of any one of clauses 751 to 777 wherein the path-following removable patch portion consists of removable patch portions of the one or more first removable patch portions, the one or more second removable patch portions, and/or the one or more third removable patch portions.

Clause 779. The adhesive patch of any of clauses 654 to 778, wherein at least one, or each, releasably coupled pair of portions is releasably coupled by a releasable coupling.

Clause 780. The adhesive patch of any of clauses 654 to 779, wherein the releasable coupling comprises or consists of at least one line of weakness or frangible boundary.

Clause 781. The adhesive patch of clause 780, wherein the releasable coupling delineates an otherwise continuous portion into the two releasably coupled portions.

Clause 782. The adhesive patch of any one of clauses 779 to 781, wherein the releasable coupling is at least partly formed by at least part of the adhesive layer of one of the pair of releasably coupled patch portions contacting the upper surface of the other of the pair of releasably coupled patch portions.

Clause 783. The adhesive patch of any one of clauses 779 to 782, wherein the releasable coupling is at least partly, or entirely, formed by perforations.

Clause 784. The adhesive patch of any one of clauses 779 to 783, wherein the releasable coupling is at least partly, or entirely, formed by a thinning of the material forming the pair of releasably coupled portions.

Clause 785. The adhesive patch of clause 784, wherein the thinning is through an upper surface of the material, such that the upper surface along at least part of the releasable coupling is relatively lower than the upper surface of the material immediately away from the releasable coupling.

Clause 786. The adhesive patch of clause 784 or 785, wherein the thinning is through a lower surface of the material, such that the lower surface along at least part of the releasable coupling is relatively higher than the lower surface of the material immediately away from the releasable coupling.

Clause 787. The adhesive patch of any one of clauses 784 to 786 wherein the thinning results in at least part or all of the releasable coupling having a thickness that is less than on either immediate side of the releasable coupling.

Clause 788. The adhesive patch of any one of clauses 779 to 787, wherein the releasable coupling is at least partly, or entirely, formed by an adhesive coupling two or more otherwise non-contacting edges of the pair of releasably coupled portions.

Clause 789. The adhesive patch of any one of clauses 759 to 788 wherein a shared lower surface of at least one, or each, pair of releasably coupled portions comprises no adhesive layer along at least part, or all, of the releasable coupling.

Clause 790. The adhesive patch of any one of clauses 759 to 789, wherein the shared lower surface of at least one, or each, pair of releasably coupled portions comprises a relatively weaker adhesive layer along at least part, or all, of the releasable coupling.

Clause 791. The adhesive patch of any of clauses 654 to 790, wherein a shared lower surface of at least one, or each, pair of releasably coupled portions comprises no adhesive layer or a weaker adhesive along at least part, or all, of the releasable coupling when compared to the adhesive layer.

Clause 792. The adhesive patch of any of clauses 654 to 791, wherein the outer periphery of the primary patch portion and/or an outer periphery of at least one of the one or more removable patch portions defines at least one outside corner.

Clause 793. The adhesive patch of clause 792, wherein at least one, or all, of the outside corners has an external angle less than 315 degrees, optionally less than 225 degrees.

Clause 794. The adhesive patch of clause 792 or 793, wherein at least one, or all, of the outside corners is blunt.

Clause 795. The adhesive patch of any one of clauses 792 to 794, wherein at least one, or all, of the outside corners is radiused with a radius of at least 0.3 mm or at least 3 mm.

Clause 796. The adhesive patch of any one of clauses 792 to 795 wherein any outside corner having an external angle greater than 315 degrees has a radius of at least 0.5 mm and/or wherein any outside corner having an external angle less than 225 may have no radius.

Clause 797. The adhesive patch of any of clauses 654 to 796, wherein at least one of the one or more removable portions tessellates with an adjacent one of plurality of patch portions.

Clause 798. The adhesive patch of any of clauses 654 to 797, wherein each one of the one or more removable portion tessellates with an adjacent one of the primary patch portion and/or another of the one or more removable patch portions.

Clause 799. The adhesive patch of any of clauses 654 to 798, wherein each one of the one or more removable patch portions tessellates with all adjacent ones of the plurality of patch portions.

Clause 800. The adhesive patch of any of clauses 654 to 799, wherein, before any of the one or more removable patch portions have been removed, no gaps exist between any adjacent members of the plurality of patch portions.

Clause 801. The adhesive patch of any of clauses 654 to 800, wherein at least one, or all, of the one or more removable patch portions comprises a pull tab, optionally wherein one or all of the pull tabs comprises no adhesive layer on its lower surface.

Clause 802. The adhesive patch of clause 801, wherein the pull tab extends from an edge of the removable portion and/or from the upper surface of the removable patch portion.

Clause 803. The adhesive patch of any of clauses 654 to 802, wherein the adhesive patch is configured so that if an edge forming part of the outer patch periphery starts to peel from or become improperly adhered to the user's body, one or more removable patch portions defining that edge can be removed to expose an edge that is not starting to peel from or is properly adhered to the user's body.

Clause 804. The adhesive patch of any of clauses 654 to 803, wherein at least one of the one or more removable patch portions comprises a partial releasable coupling extending from an edge of the removable portion to a point within the removable portion, wherein the partial releasable coupling assists in removal of the removable portion.

Clause 805. The adhesive patch of clause 804, wherein the partial releasable coupling extends substantially orthogonally from the edge of the removable portion.

Clause 806. The adhesive patch of clause 804, wherein the partial releasable coupling extends substantially non-orthogonally from the edge of the removable portion.

Clause 807. The adhesive patch of any one of clauses 804 to 806, wherein the primary patch portion and the one or more removable patch portions define an outer patch periphery, wherein the adhesive patch comprises at least one releasable coupling extending from a location on the outer patch periphery to assist in removal of the one or more removable patch portions.

Clause 808. The adhesive patch of clause 807, wherein the at least one releasable coupling extends substantially orthogonally to the outer patch periphery at the location.

Clause 809. The adhesive patch of clause 808, wherein distal from the outer patch periphery, the at least one releasable coupling extends non-orthogonally to or parallel with the edge of the outer patch periphery at the location.

Clause 810. The adhesive patch of clause 807, wherein the at least one releasable coupling extends substantially non-orthogonally from the outer patch periphery at the location.

Clause 811. The adhesive patch of clause 807 to 810, wherein the at least one releasable coupling is configured such that at least one removable portion adjacent to the releasable coupling is configured to be removed in a circumferential direction and/or parallel with the outer patch periphery proximal to that location.

Clause 812. The adhesive patch of any one of clauses 804 to 811, wherein the partial releasable coupling and/or at least one releasable coupling are according to clauses 780 to 791.

Clause 813. An on-body device patch comprising an on-body device and the adhesive patch of any one of clauses 654 to 812.

Clause 814. The on-body device patch of clause 813, wherein the on-body device is secured to a device adhesive on the upper surface of the primary patch portion of the primary patch portion.

Clause 815. The on-body device patch of clause 813 or 814, wherein the on-body device is an analyte monitor.

Clause 816. The on-body device patch of any one of clauses 813 to 815, wherein the on-body device is a glucose monitor, optionally a continuous glucose monitor.

Clause 817. The adhesive patch or on-body device patch of any of clauses 654 to 816, wherein the adhesive patch comprises an anti-stick layer.

Clause 818. The adhesive patch or on-body device patch of clause 817, wherein the anti-stick layer is located at or on the upper surface of the primary patch portion.

Clause 819. The adhesive patch or on-body device patch of clauses 817 or 818, wherein the anti-stick layer is located at or on the upper surface of the one or more removable patch portions.

Clause 820. The adhesive patch or on-body device patch of any of clauses 817 to 819, when directly or indirectly dependent on clause 813, wherein the anti-stick layer is located at or on the upper surface of the on-body device.

Clause 821. The adhesive patch or on-body device patch of any of clauses 817 to 820, wherein the anti-stick layer comprises a fluoropolymer or silicone.

Clause 822. The adhesive patch or on-body device patch of any of clauses 817 to 821, wherein the anti-stick layer is a permanent coating.

Clause 823. The adhesive patch or on-body device patch of any of clauses 817 to 821, wherein the anti-stick layer is formed in or inherent to the material of the primary patch portion, one or more removable patch portions, or on-body device.

Clause 824. The adhesive patch or on-body device patch of any of clauses 817 to 821, wherein the anti-stick layer is a removable sticker.

Clause 825. The adhesive patch or on-body device patch of clause 824, wherein the removable sticker comprises a weak adhesive and/or wherein the adhesive of the removable sticker is weaker than an, or all, adhesive layers of the adhesive patch.

Clause 826. A method of applying an adhesive patch according to any of clauses 1 to 154, 178-288, 324-610, or 654-825, the method comprising applying and adhering the adhesive patch to a user's skin.

Clause 827. The method of clause 166, wherein the adhesive patch is an on-body device patch according to any of clauses 813 to 816.

Clause 828. The method of clause 827, wherein the method further comprises using an applicator tool to apply the on-body device patch to the user's skin.

Clause 829. The method of clauses 827 or 828, wherein the method further comprises applying a further adhesive patch according to any of clauses 1 to 154, 178-288, 324-610, or 654-825 over the on-body device patch, wherein the further adhesive patch is an overpatch.

Clause 830. The method of clause 826, wherein the adhesive patch is applied over a pre-existing on-body device patch, wherein the adhesive patch is an overpatch.

Clause 831. The method of clause 829 or 831, wherein the method further comprises placing a sticker having an anti-stick upper surface onto an upper surface of the on-body device patch before applying the overpatch.

Clause 832. The method of any of clauses 829 to 831, wherein applying the overpatch comprises adhering the further adhesive patch to the skin or body surrounding the on-body device patch.

Clause 833. The method of any of clauses 829 to 832, wherein applying the overpatch comprises aligning an aperture or mating feature with the on-body device of the on-body device patch.

Clause 834. A method of monitoring an analyte using the on-body device patch according to any of clauses 813 to 816 or using an on-body device secured using the adhesive patch of any of clauses 654 to 825, the method comprising monitoring the analyte.

Clause 835. The method of monitoring an analyte according to clause 834, wherein the analyte is glucose.

Clause 836. The method of monitoring an analyte according to clause 834 or 835, wherein the analyte is monitored substantially continuously.

Clause 837. The method of monitoring an analyte according to any of clauses 834 to 836, wherein the on-body device patch and/or the adhesive patch are applied according to the method of any of clauses 826 to 833.

Clause 838. An adhesive patch assembly comprising the adhesive patch of any of clauses 654 to 812 or 817 to 825, wherein the adhesive patch assembly comprises an on-body device according to any of clauses 1 to 154, 178 to 288, or 322 to 610.

Clause 839. The on-body device patch of any of clauses 813 to 825, wherein the on-body device comprises the features of an on-body device according to any of clauses 1 to 154, 178 to 288, or 322 to 610.

Clause 840. A reinforcement device for reinforcing attachment of an adhesive patch to a skin surface of a user, the adhesive patch coupled to a housing of an on-body device, wherein the reinforcement device comprises: one or more reinforcement legs, wherein each of the one or more reinforcement legs comprises: a first end configured to be directly or indirectly coupled to the housing; and a second end configured to contact the adhesive patch.

Clause 841. The reinforcement device of clause 840, wherein the one or more reinforcement legs comprises a plurality of reinforcement legs.

Clause 842. The reinforcement device of clause 841, wherein the plurality of reinforcement legs are configured to be arranged around the perimeter of the housing when coupled directly or indirectly to the housing.

Clause 843. The reinforcement device of clause 841 or clause 842, wherein the plurality of reinforcement legs are spaced evenly around the perimeter of the housing.

Clause 844. The reinforcement device of any one of clauses 841 to clause 843, wherein each of the plurality of reinforcement legs defines a respective longitudinal axis and, wherein a projection of the respective longitudinal axis of each reinforcement leg in a plane of the skin surface is approximately aligned with a radial axis extending from a center of the housing.

Clause 845. The reinforcement device of any one of clause 841 to clause 844, wherein each of the plurality of reinforcement legs extend downwardly from the housing towards the skin surface.

Clause 846. The reinforcement device of any one of clause 841 to clause 844, wherein each of the plurality of reinforcement legs extend parallel to the adhesive patch.

Clause 847. The reinforcement device of any one of clause 841 to clause 845, wherein the second end of each reinforcement leg comprises a foot.

Clause 848. The reinforcement device of clause 847, wherein each foot is configured to be adhered to the adhesive patch.

Clause 849. The reinforcement device of clause 847 or clause 848, wherein a base surface of each foot comprises an adhesive layer.

Clause 850. The reinforcement device of any one of clause 845 to clause 849, wherein each foot is configured to extend radially beyond a periphery of the adhesive patch.

Clause 851. The reinforcement device of any one of clause 850, wherein a portion of each foot is configured to overlay the adhesive patch and a portion of each foot is configured to overlay the skin surface.

Clause 852. The reinforcement device of any one of clause 845 to clause 849, wherein an outer edge of each foot is configured to extend radially to and not beyond a periphery of the adhesive patch.

Clause 853. The reinforcement device of any one of clause 841 to clause 852, wherein the first end of each reinforcement leg is configured to be directly coupled to the housing.

Clause 854. The reinforcement device of clause 853, wherein the first end of each reinforcement leg is configured to be coupled to the housing by a respective hinge.

Clause 855. The reinforcement device of clause 854, wherein each hinge is a living hinge.

Clause 856. The reinforcement device of clause 853 or clause 854, wherein the first end of each reinforcement leg is configured to be coupled to the housing by a respective snap-fit joint.

Clause 857. The reinforcement device of any one of clause 853 to clause 855, wherein the plurality of reinforcement legs are formed integrally with the housing.

Clause 858. The reinforcement device of any one of clause 841 to clause 852, wherein the first end of each reinforcement leg is directly coupled to a body portion of the reinforcement device, wherein the body portion is configured to be coupled to the housing.

Clause 859. The reinforcement device of clause 858, wherein the first end of each reinforcement leg is configured to be coupled to the body portion by a respective hinge.

Clause 860. The reinforcement device of clause 859, wherein each hinge is a living hinge.

Clause 861. The reinforcement device of clause 858 or clause 859, wherein the first end of each reinforcement leg is configured to be coupled to the body portion by a respective snap-fit joint.

Clause 862. The reinforcement device of any one of clause 858 to clause 860, wherein the plurality of reinforcement legs are formed integrally with the body portion.

Clause 863. The reinforcement device of any one of clause 858 to clause 862, wherein the body portion is configured to be coupled to the housing by one or more snap-fit joints.

Clause 864. The reinforcement device of clause 863, wherein the one or more snap-fit joints comprises a snap-fit joint that extends around the perimeter of the housing and/or the body portion.

Clause 865. The reinforcement device of any one of clause 858 to clause 864, wherein the body portion further comprises: an inner edge defining an opening for receiving the housing; and an outer edge.

Clause 866. The reinforcement device of clause 865, wherein the body portion is formed of an elastomeric material and, wherein the inner edge corresponds to a shape and size of the housing such that the inner edge of the body portion is configured to grip the housing.

Clause 867. The reinforcement device of clause 865 or clause 866, wherein the inner edge has a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Clause 868. The reinforcement device of any one of clause 865 to clause 867, wherein the shape of the inner edge comprises sharp and/or rounded corners.

Clause 869. The reinforcement device of any one of clause 865 to clause 867, wherein the shape of the inner edge comprises straight and/or curved edges.

Clause 870. The reinforcement device of clause 869, wherein at least some of the curved edges have a constant radius of curvature.

Clause 871. The reinforcement device of clause 869 or clause 870, wherein at least some of the curved edges have a variable radius of curvature.

Clause 872. The reinforcement device of any one of clause 865 to clause 871, wherein the shape of the inner edge comprises one or more indentations and/or one or more protrusions or lobes.

Clause 873. The reinforcement device of any one of clause 858 to clause 864, wherein at least part of the body portion is configured to overlay at least part of the upper surface of the housing.

Clause 874. The reinforcement device of clause 873, wherein the entirety of the body portion is configured to overlay at least part of the upper surface of the housing.

Clause 875. The reinforcement device of clause 873 or clause 874, wherein the body portion is configured to overlay the entirety of the upper surface of the housing.

Clause 876. The reinforcement device of any of clause 873 to clause 875, wherein an underside of the body portion is configured to be adhered to the upper surface of the housing.

Clause 877. The reinforcement device of any one of clause 873 to clause 876, wherein the body portion comprises a plate, wherein an outer edge of the plate defines an outer edge of the body portion.

Clause 878. The reinforcement device of any one of clause 865 to clause 872 or clause 877, wherein the outer edge of the body portion has a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Clause 879. The reinforcement device of clause 877 or clause 878, wherein the shape of the outer edge of the body portion comprises sharp and/or rounded corners.

Clause 880. The reinforcement device of any one of clause 877 to clause 879, wherein the shape of the outer edge of the body portion comprises straight and/or curved edges.

Clause 881. The reinforcement device of clause 880, wherein at least some of the curved edges have a constant radius of curvature.

Clause 882. The reinforcement device of clause 880 or clause 881, wherein at least some of the curved edges have a variable radius of curvature.

Clause 883. The reinforcement device of any one of clause 877 to clause 882, wherein the shape of the outer edge of the body portion comprises one or more indentations and/or one or more protrusions or lobes.

Clause 884. The reinforcement device of any one of clause 858 to clause 883 wherein the body portion comprises a plurality of apertures extending entirely through the body portion.

Clause 885. The reinforcement device of any one of clause 858 to clause 884, wherein the body portion is formed form a moisture permeable material.

Clause 886. The reinforcement device of any one of clause 858 to clause 885 wherein the body portion is formed from a rigid material.

Clause 887. The reinforcement device of any one of clause 858 to clause 886 when directly or indirectly dependent on clause 858, wherein the reinforcement legs are configured to be at least partially covered by the body portion in use.

Clause 888. The reinforcement device of clause 887, wherein the reinforcement legs are configured to be fully covered by the body portion when in use.

Clause 889. The reinforcement device of any one of clause 850 to clause 888 when directly or indirectly dependent on clause 845, wherein the plurality of feet are coupled by a connection member.

Clause 890. The reinforcement device of clause 889, wherein the connection member is configured to adhere to the adhesive patch.

Clause 891. The reinforcement device of clause 889 or clause 890, wherein an underside of the connection member comprises an adhesive layer.

Clause 892. The reinforcement device of any one of clause 889 to clause 891, wherein the connection member comprises an inner edge and an outer edge.

Clause 893. The reinforcement device of clause 892, wherein the inner edge of the connection member and/or the outer edge of connection member has a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Clause 894. The reinforcement device of clause 893, wherein the shape of the inner edge of the connection member and/or the shape of the outer edge of the connection member comprises sharp and/or rounded corners.

Clause 895. The reinforcement device of clause 893 or clause 894, wherein the shape of the inner edge of the connection member and/or the shape of the outer edge of the connection member comprises straight and/or curved edges.

Clause 896. The reinforcement device of clause 895, wherein at least some of the curved edges have a constant radius of curvature.

Clause 897. The reinforcement device of clause 895 or clause 896, wherein at least some of the curved edges have a variable radius of curvature.

Clause 898. The reinforcement device of any one of clause 892 to clause 897, wherein the shape of the inner edge of the connection member and/or the shape of the outer edge of the connection member comprises one or more indentations and/or one or more protrusions or lobes.

Clause 899. The reinforcement device of any one of clause 889 to clause 898 when directly or indirectly dependent on clause 858, wherein the body portion comprises the connection member.

Clause 900. The reinforcement device of any of clause 889 to clause 899, wherein the plurality of feet are formed integrally with the connection member.

Clause 901. The reinforcement device of any of clause 840 to clause 900, wherein the reinforcement legs are formed from a rigid material.

Clause 902. The reinforcement device of any one of clause 858 to clause 901 when directly or indirectly dependent on clause 858, wherein the reinforcement legs and body portion are formed from different materials.

Clause 903. The reinforcement device of clause 902, wherein the material from which the reinforcement legs are formed from is more flexible than the material from which the body portion is formed.

Clause 904. The reinforcement device of any one of clause 889 to clause 902 when directly or indirectly dependent on clause 858 wherein the material from which the reinforcement legs are formed is more flexible than the material from which the housing is formed.

Clause 905. The reinforcement device of any one of clause 841 to clause 904 wherein each reinforcement leg is biased such that the second end exerts a force on the adhesive patch in a direction towards the skin surface.

Clause 906. The reinforcement device of clause 905, wherein the biasing is provided by a spring member.

Clause 907. The reinforcement device of clause 905 or clause 906, wherein the reinforcement legs are provided with a length such that they are configured to bend when coupled directly or indirectly to the housing.

Clause 908. The reinforcement device of any one of clause 841 to clause 907, wherein a cross section of each reinforcement leg is arcuate.

Clause 909. The reinforcement device of clause 908, wherein, when each reinforcement leg is coupled directly or indirectly to the housing, the each reinforcement leg is convex with respect to the skin surface.

Clause 910. The reinforcement device of clause 908 or clause 909, wherein the arcuate shape of each leg is configured to allow bending of the second end of each leg towards the skin surface but resist against bending of the second end of each leg away from the skin surface.

Clause 911. The reinforcement device of any of clause 840 to clause 910 comprising at least 4 reinforcement legs.

Clause 912. The reinforcement device of any of clause 840 to clause 911 comprising at least 8 reinforcement legs.

Clause 913. The reinforcement device of any of clause 840 to clause 912 comprising at least 12 reinforcement legs.

Clause 914. An analyte monitoring system, comprising: the reinforcement device of any of clause 840 to clause 913, and the on-body device.

Clause 915. The analyte monitoring system of clause 914, wherein the on-body device is a glucose monitor.

Clause 916. The analyte monitoring system of clause 915, wherein the on-body device is a continuous glucose monitor.

Clause 917. The reinforcement device of clause 858 or any of clause 859 to clause 913 when directly or indirectly dependent on clause 858, wherein the body portion comprises an anti-stick layer.

Clause 918. The reinforcement device of clause 917 wherein the anti-stick layer is located at or on an upper surface of the body portion.

Clause 919. The analyte monitoring system of any of clause 914 to clause 916 wherein the on-body device comprises an anti-stick layer.

Clause 920. The analyte monitoring system of clause 919 wherein the anti-stick layer is located at or on an upper surface of the on-body device.

Clause 921. The reinforcement device of clause 917 or clause 918, or the analyte monitoring system of clause 919 or clause 920, wherein the anti-stick layer comprises a fluoropolymer or silicone.

Clause 922. The reinforcement device of clause 917, clause 918, or clause 921, or the analyte monitoring system of clause 919, clause 920, or clause 921, wherein the anti-stick layer is a permanent coating.

Clause 923. The reinforcement device of clause 917, clause 918, clause 921, or clause 922, or the analyte monitoring system of any of clause 919 to clause 922, wherein the anti-stick layer is formed in or inherent to the material of the body portion or on-body device.

Clause 924. The reinforcement device of clause 917, clause 918, or any of clause 921 to clause 923, or the analyte monitoring system of any of clause 919 to clause 923, wherein the anti-stick layer is a removable sticker.

Clause 925. The reinforcement device or analyte monitoring system of clause 924, wherein the removable sticker comprises a weak adhesive.

Clause 926. A reinforcement device for reinforcing the attachment of an adhesive patch to a skin surface of a user, the adhesive patch coupled to a housing of an on-body device, the reinforcement device comprising: one or more reinforcement bands, wherein each of the one or more reinforcement bands comprises: an inner edge defining an opening for receiving the housing; and a lower surface configured to overlay the adhesive patch.

Clause 927. The reinforcement device of clause 926, wherein each of the one or more reinforcement bands comprises an outer edge.

Clause 928. The reinforcement device of clause 927, wherein the outer edge is configured to extend radially to a peripheral edge of the adhesive patch such that each of the one or more reinforcement bands reinforces the peripheral edge of the adhesive patch.

Clause 929. The reinforcement device of clause 927, wherein the outer edge is configured to extend radially beyond a peripheral edge of the adhesive patch such that each of the one or more reinforcement bands reinforces the peripheral edge of the adhesive patch.

Clause 930. The reinforcement device of any of clauses 926 to 929, wherein the lower surface comprises an adhesive layer configured to adhere the lower surface to the skin surface.

Clause 931. The reinforcement device of any of clauses 926 to 930, wherein the inner edge is configured to exert a force on the housing and thereby grip the housing when in use.

Clause 932. The reinforcement device of clause 931, wherein at least one of the one or more reinforcement bands is configured to mechanically couple to the housing.

Clause 933. The reinforcement device of any of clauses 926 to 932, wherein at least one of the one or more reinforcement bands is formed from an elastomeric material.

Clause 934. The reinforcement device of any of clauses 926 to 933, wherein at least one of the one or more reinforcement bands is formed from a shape-memory material.

Clause 935. The reinforcement device of any of clauses 926 to 934, wherein at least one of the one or more reinforcement bands is rigid, optionally wherein the at least one of the one of more reinforcement bands is formed from a rigid polymer.

Clause 936. The reinforcement device of any of clauses 926 to 935, wherein the opening is configured such that an entire upper surface of the housing can extend through the opening.

Clause 937. The reinforcement device of any of clauses 926 to 936, wherein the one or more reinforcement bands comprises a plurality of reinforcement bands.

Clause 938. The reinforcement device of clause 937, wherein the plurality of reinforcement bands are stacked on top of one another such that the lower surface of a second reinforcement band of the plurality of reinforcement bands contacts an upper surface of a first reinforcement band of the plurality of reinforcement bands.

Clause 939. The reinforcement device of clause 937 or clause 938, wherein each of the plurality of reinforcement bands are stacked parallel to an axis of the housing.

Clause 940. The reinforcement device of clause 937 when dependent on clause 927, wherein each of the plurality of reinforcement bands are arranged in a concentric manner such that the inner edge of a second reinforcement band of the plurality of reinforcement bands contacts or overlaps with an outer edge of a first reinforcement band of the plurality of reinforcement bands.

Clause 941. The reinforcement device of any one of clauses 937 to 940, wherein each of the plurality of reinforcement bands is frangibly connected to a neighbouring one of the plurality of reinforcement bands.

Clause 942. The reinforcement device of clause 941 when dependent on clause 940, wherein the inner edge of the second reinforcement band is frangibly connected to the outer edge of the first reinforcement band such that, in use, the second reinforcement band is removable from the skin surface of the user while the first reinforcement band remains in place.

Clause 943. The reinforcement device of clause 942 further comprising a third reinforcement band, wherein the inner edge of the third reinforcement band is frangibly connected to the outer edge of the first reinforcement band such that, in use, the third reinforcement band is removable from the skin surface of the user while the second reinforcement band remains in place.

Clause 944. The reinforcement device of clause 941 when dependent on clause 938, wherein the lower surface of the second reinforcement band is frangibly connected to the upper surface of the first reinforcement band such that, in use, the second reinforcement band is removable from the skin surface of the user while the first reinforcement band remains in place.

Clause 945. The reinforcement device of clause 944 further comprising a third reinforcement band, wherein the lower surface of the third reinforcement band is frangibly connected to the upper surface of the second reinforcement band such that, in use, the third reinforcement band is removable from the skin surface of the user while the second reinforcement band remains in place.

Clause 946. The reinforcement device of any one of clauses 941 to 945, wherein a diameter of the outer edge of the second reinforcement band is greater than a diameter of the outer edge of the first reinforcement band.

Clause 947. The reinforcement device of clause 946 when dependent on clause 943 or clause 945, wherein a diameter of the outer edge of the third reinforcement band is greater than a diameter of the outer edge of the second reinforcement band.

Clause 948. The reinforcement device of clause 937, wherein each of the reinforcement bands are arranged concentrically and the reinforcement bands do not overlap with each other.

Clause 949. The reinforcement device of clause 948, wherein a ratio of a height of each reinforcement band to a width of the each reinforcement band is greater than 1, wherein the height of each reinforcement band is defined between the lower surface of each reinforcement band and an upper surface of each reinforcement band, and, wherein the width of each reinforcement band is defined between the inner and outer edges of each reinforcement band

Clause 950. The reinforcement device of clause 949, wherein the ratio of the height of each reinforcement band to the width of the each reinforcement band is greater than 2.

Clause 951. The reinforcement device of clause 949, wherein the ratio of the height of each reinforcement band to the width of the each reinforcement band is greater than 3.

Clause 952. The reinforcement device of any one of clauses 948 to 951, wherein each of the plurality of bands are connected by one or more webbing members.

Clause 953. The reinforcement device of clause 952 wherein the webbing members extend radially between the concentric bands.

Clause 954. The reinforcement device of any one of clauses 948 to 953 wherein the plurality of concentric bands are covered by a guard band.

Clause 955. The reinforcement device of any one of clauses 937 to 954, wherein at least two of the plurality of reinforcement bands are formed of at least two different materials.

Clause 956. The reinforcement device of clause 955, wherein the materials are selected to provide differing rigidity, adhesion, and/or moisture wicking properties.

Clause 957. The reinforcement device of clause 956, wherein the rigidity of the plurality of reinforcement bands decreases in a radially outward direction.

Clause 958. The reinforcement device of clause 956 or clause 957 when dependent on clause 930, wherein the adhesion of the plurality of reinforcement bands decreases in a radially outward direction.

Clause 959. The reinforcement device of clause 956 or clause 957 when dependent on clause 930, wherein the adhesion of the plurality of reinforcement bands increases in a radially outward direction.

Clause 960. The reinforcement device of any one of clauses 956 to 959, wherein the moisture wicking ability of the material decreases in a direction away from the skin surface.

Clause 961. The reinforcement device of any one of clauses 926 to 936, wherein a first reinforcement band of the one or more reinforcement bands comprises a plurality of radially extending channels each defining a gap between the adhesive patch and the first reinforcement band.

Clause 962. The reinforcement device of clause 961, wherein each of the plurality of radially extending channels extends from the inner edge of the first reinforcement band to the outer edge of the first reinforcement band such that each gap extends from the inner edge of the first reinforcement band to the outer edge of the first reinforcement band.

Clause 963. The reinforcement device of clause 961 or clause 962, wherein each of the plurality of channels is configured to allow convex curving of the first reinforcement band and resist concave curving of the first reinforcement band.

Clause 964. The reinforcement band device of any one of clauses 961 to 963, wherein a cross section of each channel along a plane perpendicular to a longitudinal axis of the each channel is arcuate.

Clause 965. The reinforcement device of any one of clauses 926 to 930, wherein the one or more reinforcement bands comprise a plurality of discrete beads, wherein the plurality of discrete beads are arranged in a plurality of radially extending rows of beads, wherein each row of beads is configured such that, when an adjacent area of the adhesive patch begins to peel away from the skin surface, each bead abuts against its neighbour, thus preventing further peeling of the adhesive patch.

Clause 966. The reinforcement device of clause 965, wherein each row of beads comprises at least three beads.

Clause 967. The reinforcement device of clause 965 or clause 965, wherein a face of each bead that is configured to abut against a neighbouring bead is flat.

Clause 968. The reinforcement device of any one of clauses 965 to 967, wherein each bead has the shape of a prism and is arranged such that a longitudinal axis of each prism is oriented parallel to the row of beads of which the each bead forms part, optionally wherein each bead is cuboidal.

Clause 969. The reinforcement device of any of clauses 926 to 964, wherein at least one of the one or more reinforcement bands comprises a plurality of apertures extending entirely through the reinforcement band.

Clause 970. The reinforcement device of clause 969, wherein each aperture of the plurality of apertures has an oval shape.

Clause 971. The reinforcement device of clause 970, wherein a minor axis of each aperture of the plurality of apertures is approximately aligned with a radial axis extending from a center of the opening.

Clause 972. The reinforcement device of any of clauses 926 to 971, wherein the inner edge of each of the one or more reinforcement bands has a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Clause 973. The reinforcement device of any of clauses 926 to 972, wherein the shape of the inner edge of each of the one or more reinforcement bands comprises sharp and/or rounded corners.

Clause 974. The reinforcement device of any of clauses 926 to 973, wherein the shape of the inner edge of each of the one or more reinforcement bands comprises straight and/or curved edges.

Clause 975. The reinforcement device of clause 974, wherein at least some of the curved edges have a constant radius of curvature.

Clause 976. The reinforcement device of clause 974 or clause 975, wherein at least some of the curved edges have a variable radius of curvature.

Clause 977. The reinforcement device of any of clauses 926 to 976, wherein the shape of the inner edge of each of the one or more reinforcement bands comprises one or more indentations and/or one or more protrusions or lobes.

Clause 978. The reinforcement device of any of clauses 926 to 977, wherein the outer edge of each of the one or more reinforcement bands has a circular shape; a truncated circular shape; a semi-circular shape; an elliptical shape; an annular shape; an teardrop shape; a crescent shape; a triangular shape; a pill shape; a dumbbell shape; a square shape; a rectangular shape; a trapezium shape; a parallelogram shape; a pentagon shape; a hexagon shape; a heptagon shape; an octagon shape; a nonagonal shape; a decagonal shape; a hendecagonal shape; or a dodecagonal shape.

Clause 979. The reinforcement device of any of clauses 926 to 978, wherein the shape of the outer edge of each of the one or more reinforcement bands comprises sharp and/or rounded corners.

Clause 980. The reinforcement device of any of clauses 926 to 979, wherein the shape of the outer edge of each of the one or more reinforcement bands comprises straight and/or curved edges.

Clause 981. The reinforcement device of clause 980, wherein at least some of the curved edges have a constant radius of curvature.

Clause 982. The reinforcement device of clause 979 or clause 980, wherein at least some of the curved edges have a variable radius of curvature.

Clause 983. The reinforcement device of any of clauses 926 to 982, wherein the shape of the outer edge of each of the one or more reinforcement bands comprises one or more indentations and/or one or more protrusions or lobes.

Clause 984. The reinforcement device of any of clauses 926 to 983, wherein the shape of the outer edge of each of the one or more reinforcement bands corresponds to the shape of the inner edge of each of the one or more reinforcement bands.

Clause 985. The reinforcement device of any of clauses 926 to 984, wherein the shape of the inner edge of each of the one or more reinforcement bands corresponds to a shape of an outer edge of housing.

Clause 986. The reinforcement device of any of clauses 926 to 985, wherein the lower surface of each of the one or more reinforcement bands is configured to lie parallel to the skin surface.

Clause 987. An analyte monitoring system, comprising: the reinforcement device of any of clauses 926 to 986; and the on-body device.

Clause 988. The analyte monitoring system of clause 987, wherein the on-body device comprises: the housing; an analyte sensor comprising a first portion arranged within the housing and a second portion configured to be positioned below a skin surface and in contact with a bodily fluid to generate a signal indicative of an analyte level of a user; and sensor electronics arranged within the housing and coupled to the first portion of the analyte sensor, wherein the sensor electronics comprise a processor and wireless communications circuitry, wherein the adhesive patch is coupled to a first side of the housing and configured to secure the first side of the housing to the skin surface of the user.

Clause 989. The analyte monitoring system of clause 988, wherein the analyte is one or more of: glucose, ammonium, asparagine, aspartate, cholesterol, choline, creatinine, diaphorase inhibitors, ethanol, glutamate, glycerol, one or more ketones, lactate, lactose, oxygen, phosphate, potassium, pyruvate, and uric acid.

Clause 990. The analyte monitoring system of clause 989, wherein the on-body device is a glucose monitor.

Clause 991. The analyte monitoring system of clause 990, wherein the on-body device is a glucose monitor.

Clause 992. The reinforcement device of any of clauses 926 to 986, wherein at least one of the one or more reinforcement bands comprises an anti-stick layer.

Clause 993. The reinforcement device of clause 992 wherein the anti-stick layer is located at or on an upper surface of the one or more reinforcement bands.

Clause 994. The analyte monitoring system of any of clauses 987 to 991 wherein the on-body device comprises an anti-stick layer.

Clause 995. The analyte monitoring system of clause 994 wherein the anti-stick layer is located at or on an upper surface of the on-body device.

Clause 996. The reinforcement device of clause 992 or clause 993, or the analyte monitoring system of clause 994 or clause 995, wherein the anti-stick layer comprises a fluoropolymer or silicone.

Clause 997. The reinforcement device of clause 992, clause 993, or clause 996, or the analyte monitoring system of clause 994, clause 995, or clause 996, wherein the anti-stick layer is a permanent coating.

Clause 998. The reinforcement device of clause 992, clause 993, clause 996, or clause 997, or the analyte monitoring system of any of clauses 994 to 997, wherein the anti-stick layer is formed in or inherent to the material of the reinforcement ring or the on-body device.

Clause 999. The reinforcement device of clause 992, clause 993, or any of clauses 996 to 998, or the analyte monitoring system of any of clauses 994 to 998, wherein the anti-stick layer is a removable sticker.

Clause 1000. The reinforcement device or analyte monitoring system of clause 999, wherein the removable sticker comprises a weak adhesive.

Clause 1001. A reinforcement assembly comprising the reinforcement device of any of clauses 840 to 913, 917 to 918, 921 to 986, 992 to 993, or 996 to 1000, and the adhesive patch of any of clauses 654 to 812 or 817 to 825.

Clause 1002. An analyte monitoring system comprising the analyte monitoring system of any of clauses 914 to 916, 919 to 925, 987 to 991, or 994 to 1000 and the adhesive patch of any of clauses 654 to 812 or 817 to 825.

Clause 1003. An analyte monitoring system comprising the reinforcement device of any of clauses 840 to 913, 917 to 918, 921 to 986, 992 to 993, or 996 to 1000, and the on-body device of any of clauses 1 to 610.

Clause 1004. The analyte monitoring system of clause 1003 further comprising the adhesive patch of any of clauses 914 to 916, 919 to 925, 987 to 991, or 994 to 1000.

Clause 1005. An on-body device for monitoring an analyte, wherein the on-body device comprises an anti-stick layer on an upper surface of the device.

Clause 1006. The on-body device of clause 1005, wherein the anti-stick layer is a coating on the upper surface of the device.

Clause 1007. The on-body device of clause 1005, wherein the anti-stick layer is a removable sticker located on the upper surface of the device.

Clause 1008. The on-body device of any of clauses 1005 to 1007, wherein the anti-stick layer comprises a fluoropolymer or silicone and/or is inherent to the material from which the on-body device is constructed or formed.

Clause 1009. The on-body device of any of clauses 1005 to 1008, wherein the on-body device is, or has the features of, the on-body device of any preceding clause.

Clause 1010. An adhesive patch for an on-body device, wherein an upper surface of the adhesive patch comprises an anti-stick layer.

Clause 1011. The adhesive patch of clause 1010, wherein the anti-stick layer is a coating on the upper surface of the adhesive patch.

Clause 1012. The adhesive patch of clause 1008, wherein the anti-stick layer is a removable sticker located on the upper surface of the adhesive patch.

Clause 1013. The adhesive patch of any of clauses 1010 to 1012, wherein the anti-stick layer comprises a fluoropolymer or silicone and/or is inherent to the material from which the adhesive patch is constructed or formed.

Clause 1014. An adhesive patch assembly comprising the adhesive patch of any of clauses 1010 to 1013 and an on-body device of any preceding clause that recites an on-body device.

Clause 1015. An adhesive patch for use with an on-body device, the adhesive patch comprising: a non-isotropic layer comprising a plurality of elongate tensile resistant regions; and an adhesive layer adhered to the non-isotropic layer.

Clause 1016. The adhesive patch of clause 1015, wherein the non-isotropic layer has a first axis along a first lateral dimension, a second axis along a second lateral dimension wherein the second axis is orthogonal to the first axis, and a height, and wherein the plurality of elongate tensile resistant regions are aligned parallel to the first axis and not the second axis.

Clause 1017. The adhesive patch of clause 1016, wherein the non-isotropic layer does not include a plurality of elongate tensile resistant regions aligned parallel to the second axis.

Clause 1018. The adhesive patch of any of clauses 1015-1016, wherein each of the plurality of elongate tensile resistant regions comprises an elongate fiber or thread.

Clause 1019. The adhesive patch of any of clauses 1015-1017, wherein the non-isotropic layer is a non-woven material, optionally comprising spunlace fibers.

Clause 1020. The adhesive patch of any of clauses 1015-1019, wherein each elongate tensile resistant region of the plurality of elongate tensile resistant regions is adjacent a second region of relatively lower tensile resistance.

Clause 1021. The adhesive patch of clause 1020 when dependent on clause 1019, wherein each elongate tensile resistant region of the plurality of elongate tensile resistant regions comprises relatively more spunlace fibers than an adjacent second region.

Clause 1022. The adhesive patch of clause 1020 when dependent on clause 1019, wherein each elongate tensile resistant region of the plurality of elongate tensile resistant regions comprises a relatively greater density of spunlace fibers than an adjacent second region.

Clause 1023. The adhesive patch of clause 1020, wherein each elongate tensile resistant region of the plurality of elongate tensile resistant regions is relatively thicker than an adjacent second region.

Clause 1024. The adhesive patch of any of clauses 1015-1023, further comprising a first opening and at least one second opening, wherein the first opening is configured to permit a housing of an on-body device to pass therethrough, and the second opening segments at least one elongate tensile resistant region of the plurality of elongate tensile resistant regions.

Clause 1025. The adhesive patch of clause 1024 when directly or indirectly dependent on clause 2, wherein the second opening is oriented transverse to the first axis.

Clause 1026. The adhesive patch of any of clauses 1024-1025, wherein the second opening is a slit.

Clause 1027. The adhesive patch of clause 1026, wherein the slit is straight.

Clause 1028. The adhesive patch of clause 1026, wherein the slit is curved.

Clause 1029. The adhesive patch of clause 1026, wherein the slit has a pattern in the form of a wave or sawtooth.

Clause 1030. The adhesive patch of any of clauses 1024-1029, comprising a plurality of the second openings.

Clause 1031. The adhesive patch of clause 1030, wherein each second opening of the plurality of second openings is arranged at a different radial orientation around a center of the adhesive patch.

Clause 1032. The adhesive patch of clause 1031, wherein each second opening of the plurality of second openings is arranged at a different radial orientation around a center of the adhesive patch in symmetrical fashion.

Clause 1033. The adhesive patch of clause 1031, wherein each second opening of the plurality of second openings is on a first side of the adhesive patch.

Clause 1034. The adhesive patch of any of clauses 1015-1033, wherein the non-isotropic layer comprises a polymeric material.

Clause 1035. The adhesive patch of any of clauses 1015-1034, further comprising a polymeric layer adhered to the non-isotropic layer.

Clause 1036. The adhesive patch of clause 1034, wherein the polymeric layer comprises polyester, polyurethane, polyethylene, polyether, elastane, polyamide, or a combination of two or more thereof.

Clause 1037. The adhesive patch of clause 1034, wherein the polymeric layer comprises polyester and/or polyurethane.

Clause 1038. The adhesive patch of any of clauses 1035-1037, wherein the polymeric layer is elastic.

Clause 1039. The adhesive patch of any of clauses 1015-1038, wherein the non-isotropic layer has a thickness of 11 mils+/−25%.

Clause 1040. The adhesive patch of clause 1039, wherein a thickness of an adhesive layer on a skin-facing side of the non-isotropic layer is 4 mils+/−25%.

Clause 1041. The adhesive patch of any of clauses 1039-1040 when dependent, directly or indirectly, on any of clauses 1035-1037B, wherein the polymeric layer has a thickness of 0.8 mils+/−25%.

Clause 1042. The adhesive patch of clause 1041, wherein an adhesive layer between the non-isotropic layer and the polymeric layer has a thickness of one mil+/−25%.

Clause 1043. The adhesive patch of any of clauses 1015-1038, wherein the non-isotropic layer has a thickness of 11 mils+/−10%.

Clause 1044. The adhesive patch of clause 1039, wherein a thickness of an adhesive layer on a skin-facing side of the non-isotropic layer is 4 mils+/−10%.

Clause 1045. The adhesive patch of any of clauses 1043-1044 when dependent, directly or indirectly, on any of clauses 1035-1038, wherein the polymeric layer has a thickness of 0.8 mils+/−10%.

Clause 1046. The adhesive patch of clause 1045, wherein an adhesive layer between the non-isotropic layer and the polymeric layer has a thickness of one mil+/−10%.

Clause 1047. The adhesive patch of any of clauses 1015-1046, comprising an adhesive layer on an upper surface configured to adhere to a housing of an on-body device.

Clause 1048. The adhesive patch of any of clauses 1015-1046, configured for application over at least a portion of an on-body device comprising a housing and a different adhesive patch.

Clause 1049. The adhesive patch of any of clauses 1015-1046, configured as a main patch adhered to an underside of a housing of an on-body device.

Clause 1050. The adhesive patch of any of clauses 1015-1046, configured as an overpatch.

Clause 1051. The adhesive patch of any of clauses 1015-1050, wherein the on-body device is configured for analyte monitoring.

Clause 1052. An on-body device configured to monitor an analyte of a body, comprising: an on-body device housing comprising electronics configured to interface with an analyte sensor; an adhesive patch adhered to the on-body device housing, wherein the adhesive patch is configured in accordance with the adhesive patch of any of clauses 1015-1051.

Clause 1053. An analyte monitoring system, comprising: an on-body device comprising a housing comprising electronics configured to interface with an analyte sensor and a first adhesive patch adhered to the on-body device housing; and a second adhesive patch configured to be applied over the housing and the first adhesive patch, wherein either or both of the first adhesive patch and second adhesive patch are configured in accordance with any of clauses 1015-1051.

Clause 1054. A method of applying an on-body device to a body, comprising: applying an adhesive patch to skin of the body, wherein the adhesive patch is coupled to an on-body device housing, wherein the adhesive patch is configured in accordance with the adhesive patch of any of clauses 1015-1051.

Clause 1055. A method of applying an overpatch to a body, comprising: applying the overpatch over an on-body device and onto skin of the body, wherein the overpatch is configured in accordance with the adhesive patch of any of clauses 1015-1051.

Clause 1056. An overpatch assembly comprising: an overpatch comprising a backing and an adhesive layer, wherein the overpatch has an opening therethrough configured to receive an on-body device; and a removable first liner adhered to the adhesive layer.

Clause 1057. The overpatch assembly of clause 1056, further comprising a support structure in the opening of the overpatch, the support structure having a recess configured to receive the on-body device.

Clause 1058. The overpatch assembly of clause 1057, wherein the housing of the on-body device has a peripheral shape that is not rotationally symmetric, and wherein the recess of the support structure has a peripheral shape corresponding to the peripheral shape of the on-body device.

Clause 1059. The overpatch assembly of any of clauses 1057-1058, wherein the support structure comprises a first physical alignment feature adapted to interface with a second physical alignment feature of the on-body device.

Clause 1060. The overpatch assembly of clause 1059, wherein the first and second physical alignment features interface in a male and female configuration.

Clause 1061. The overpatch assembly of any of clauses 1057-1060, wherein the support structure comprises at least one aperture or open region adapted to allow moisture to vent from the on-body device through the support structure.

Clause 1062. The overpatch assembly of any of clauses 1057-1060, wherein the support structure comprises a plurality of apertures or open regions adapted to allow moisture to vent from a channel terminations and/or vent outlets of the on-body device and through the support structure.

Clause 1063. The overpatch assembly of any of clauses 1056-1062, wherein the first liner comprises a pull tab portion.

Clause 1064. The overpatch assembly of clause 1063, wherein the first pull tab portion extends into the opening of the overpatch.

Clause 1065. The overpatch assembly of any of clauses 1056-1064, further comprising at least one secondary adhesive liner adhered to the adhesive layer, wherein the at least one secondary adhesive liner comprises a pull tab portion.

Clause 1066. The overpatch assembly of clause 1065, wherein the at least one secondary adhesive liner is adhered to the adhesive layer in a folded over configuration.

Clause 1067. The overpatch assembly of any of clauses 1056-1066, wherein the overpatch assembly does not include a separate and discrete removable stiffener layer.

Clause 1068. The overpatch assembly of any of clauses 1056-1066, wherein the overpatch assembly comprises a removable stiffener layer.

Clause 1069. The overpatch assembly of clause 1068, further comprising a removable stiffener unit that comprises the stiffener layer adhesively coupled to an upper liner.

Clause 1070. The overpatch assembly of any of clauses 1056-1064, wherein the adhesive layer is a first adhesive layer, the overpatch assembly further comprising a stiffener layer and a second adhesive layer adhering the stiffener layer to the backing, wherein a degree of adhesion of the stiffener layer to the second adhesive layer, a degree of adhesion of the second adhesive layer to the backing, and a degree of adhesion of the backing to the first adhesive layer are all greater than a degree of adhesion of the removable first liner to the first adhesive layer.

Clause 1071. The overpatch assembly of clause 1070, wherein at least one of the degree of adhesion of the stiffener layer to the second adhesive layer and the degree of adhesion of the second adhesive layer to the backing, is less than the degree of adhesion of the backing to the first adhesive layer and the degree of adhesion of the first adhesive layer to skin.

Clause 1072. The overpatch assembly of clause 1071, further comprising an upper liner and a third adhesive layer that couples the upper liner to the stiffener layer, wherein a degree of adhesion of the upper liner to the third adhesive layer and a degree of adhesion of the third adhesive layer to the stiffener layer is greater than at least one of the degree of adhesion of the stiffener layer to the second adhesive layer and the degree of adhesion of the second adhesive layer to the backing.

Clause 1073. The overpatch assembly of any of clauses 1057-1072, wherein the support structure comprises a first retainer structure, a sidewall, and a second retainer structure.

Clause 1074. The overpatch assembly of clause 1073, wherein the first retainer structure is configured for placement against a first side of the on-body device and the second retainer structure is configured for placement against skin of the wearer.

Clause 1075. The overpatch assembly of any of clauses 1073 or 1074, wherein the first retainer structure comprises an opening.

Clause 1076. The overpatch assembly of any of clauses 1073-1075, wherein the first retainer structure is configured as a lip.

Clause 1077. The overpatch assembly of any of clauses 1073-1076, wherein the second retainer structure comprises at least one extension section located adjacent to at least one gap region.

Clause 1078. The overpatch assembly of any of clauses 1073-1076, wherein the second retainer structure comprises a plurality of extension sections with a plurality of gap regions located therebetween.

Clause 1079. The overpatch assembly of any of clauses 1077-1078, wherein the first liner covers a first portion of the adhesive layer and the second retainer structure.

Clause 1080. The overpatch assembly of clause 1079, wherein the first portion of the adhesive layer is adhered to the skin through at least one gap region.

Clause 1081. The overpatch assembly of any of clauses 1056-1080, wherein the adhesive layer is a skin adhesive layer, wherein the removable first liner is folded, and wherein the overpatch assembly further comprises a removable second liner that is folded and adhered to the skin adhesive layer.

Clause 1082. The overpatch assembly of clause 1081, wherein a first portion of the skin adhesive layer is exposed with the first and second folded liners adhered in place over the skin adhesive layer.

Clause 1083. The overpatch assembly of clause 1082, wherein the first portion of the skin adhesive layer is exposed in a gap between the first and second folded liners in place over the skin adhesive layer.

Clause 1084. The overpatch assembly of clause 1083, wherein the gap is present across an entire width of the skin adhesive layer.

Clause 1085. The overpatch assembly of any of clauses 1081-1084, wherein at least one of the first and second folded liners comprises at least one open region through which the skin adhesive layer is exposed.

Clause 1086. The overpatch assembly of clause 1085, wherein at least one open region is intersected by a crease in the at least one of the first and second folded liners.

Clause 1087. The overpatch assembly of any of clauses 1081-1086, wherein the backing and skin adhesive layer have an interior space configured to configured to allow passage of a housing of an on-body device therethrough.

Clause 1088. The overpatch assembly of clause 1087, wherein the interior space has a first perimeter edge, wherein a portion of the removable first liner that is adhered to the skin adhesive layer has a second perimeter edge adjacent to and not aligned with the first perimeter edge.

Clause 1089. The overpatch assembly of clause 1088, wherein a portion of the removable second liner that is adhered to the skin adhesive layer has a third perimeter edge adjacent to and not aligned with the first perimeter edge.

Clause 1090. The overpatch assembly of clause 1087, wherein the backing and skin adhesive layer have an edge along the interior space and the removable first and second liners are not adhered to at least a portion of the edge.

Clause 1091. The overpatch assembly of clause 1090, wherein the removable first and second liners are not adhered to any portion of the edge.

Clause 1092. The overpatch assembly of any of clauses 1087-1091, wherein the removable first liner comprises a first enclosed open region when in an unfolded state and the removable second liner comprises a second enclosed open region when in an unfolded state.

Clause 1093. The overpatch assembly of clause 1092, wherein the first and second enclosed open regions are asymmetrical such that when the removable first and second liners are in a folded state, both folded portions of each of the removable first and second liners are adhered to the skin adhesive layer.

Clause 1094. The overpatch assembly of clause 1093, wherein the first and second enclosed open regions are each wider than the interior space.

Clause 1095. The overpatch assembly of clause 1093, wherein the first and second enclosed open regions are each wider than the interior space in in all directions of a two coordinate plane.

Clause 1096. The overpatch assembly of clause 1093, wherein the first and second enclosed open regions have a symmetric shape.

Clause 1097. The overpatch assembly of clause 1093, wherein the first and second enclosed open regions have an asymmetric shape.

Clause 1098. The overpatch assembly of clause 1093, wherein the first and second enclosed open regions each have at least one side that is tapered.

Clause 1099. The overpatch assembly of any of clauses 1093-1098, wherein the first and second enclosed open regions permit at least a portion of the skin adhesive layer to be exposed when the removable first and second liners are folded and in contact with the skin adhesive layer.

Clause 1100. The overpatch assembly of any of clauses 1056-1099, wherein the overpatch comprises at least one layer that is a non-isotropic layer.

Clause 1101. The overpatch assembly of clause 1100, wherein the backing comprises the non-isotropic layer and wherein the adhesive layer is adhered to the non-isotropic layer.

Clause 1102. The overpatch assembly of clause 1101, wherein the non-isotropic layer has a first axis along a first lateral dimension, a second axis along a second lateral dimension wherein the second axis is orthogonal to the first axis, and a height, and wherein the non-isotropic layer comprises a plurality of elongate tensile resistant regions aligned parallel to the first axis and not the second axis.

Clause 1103. The overpatch assembly of clause 1102, wherein each of the plurality of elongate tensile resistant regions comprises an elongate fiber or thread.

Clause 1104. The overpatch assembly of clause 1101, wherein the non-isotropic layer does not include a plurality of elongate tensile resistant regions aligned parallel to the second axis.

Clause 1105. The overpatch assembly of any of clauses 1100-1104, wherein the non-isotropic layer comprises spunlace fibers.

Clause 1106. The overpatch assembly of any of clauses 1102-1105, wherein each elongate tensile resistant region of the plurality of elongate tensile resistant regions is adjacent a second region of relatively lower tensile resistance.

Clause 1107. The overpatch assembly of clause 1106 when dependent on clause 1105, wherein each elongate tensile resistant region of the plurality of elongate tensile resistant regions comprises relatively more spunlace fibers than an adjacent second region.

Clause 1108. The overpatch assembly of clause 1106 when dependent on clause 1105, wherein each elongate tensile resistant region of the plurality of elongate tensile resistant regions comprises a relatively greater density of spunlace fibers than an adjacent second region.

Clause 1109. The overpatch assembly of clause 1106, wherein each elongate tensile resistant region of the plurality of elongate tensile resistant regions is relatively thicker than an adjacent second region.

Clause 1110. The overpatch assembly of any of clauses 1101-1109, further comprising a first opening and at least one second opening, wherein the first opening is configured to permit a housing of an on-body device to pass therethrough, and the second opening segments at least one elongate tensile resistant region of the plurality of elongate tensile resistant regions.

Clause 1111. The overpatch assembly of clause 1110, when directly or indirectly dependent on clause 1102, wherein the second opening is oriented transverse to the first axis.

Clause 1112. The overpatch assembly of any of clauses 1110-1111, wherein the second opening is a slit.

Clause 1113. The overpatch assembly of clause 1112, wherein the slit is straight.

Clause 1114. The overpatch assembly of clause 1112, wherein the slit is curved.

Clause 1115. The overpatch assembly of clause 1112, wherein the slit has a pattern in the form of a wave or sawtooth.

Clause 1116. The overpatch assembly of any of clauses 1110-1115, comprising a plurality of the second openings.

Clause 1117. The overpatch assembly of clause 1116, wherein each second opening of the plurality of second openings is arranged at a different radial orientation around a center of the adhesive patch.

Clause 1118. The overpatch assembly of clause 1116, wherein each second opening of the plurality of second openings is arranged at a different radial orientation around a center of the adhesive patch in symmetrical fashion.

Clause 1119. The overpatch assembly of clause 1116, wherein each second opening of the plurality of second openings is on a first side of the adhesive patch.

Clause 1120. The overpatch assembly of any of clauses 1101-1119, wherein the non-isotropic layer comprises a polymeric material.

Clause 1121. The overpatch assembly of any of clauses 1101-1120, wherein the backing further comprises a polymeric layer adhered to a non-skin-facing side of the non-isotropic layer.

Clause 1122. The overpatch assembly of clause 1121, wherein the polymeric layer comprises polyester, polyurethane, polyethylene, polyether, elastane, polyamide, or a combination of two or more thereof.

Clause 1123. The overpatch assembly of clause 1121, wherein the polymeric layer comprises polyester and/or polyurethane.

Clause 1124. The overpatch assembly of any of clauses 1121-1123, wherein the polymeric layer is elastic.

Clause 1125. The overpatch assembly of any of clauses 1101-1124, wherein the non-isotropic layer has a thickness of 11 mils+/−25%.

Clause 1126. The overpatch assembly of clause 1125, wherein a thickness of an adhesive layer on a skin-facing side of the non-isotropic layer is 4 mils+/−25%.

Clause 1127. The overpatch assembly of any of clauses 1125-1126 when dependent, directly or indirectly, on any of clauses 1121-1124, wherein the polymeric layer has a thickness of 0.8 mils+/−25%.

Clause 1128. The overpatch assembly of clause 1127, wherein an adhesive layer between the non-isotropic layer and the polymeric layer has a thickness of one mil+/−25%.

Clause 1129. The overpatch assembly of any of clauses 1101-1124, wherein the non-isotropic layer has a thickness of 11 mils+/−10%.

Clause 1130. The overpatch assembly of clause 1129, wherein a thickness of an adhesive layer on a skin-facing side of the non-isotropic layer is 4 mils+/−10%.

Clause 1131. The overpatch assembly of any of clauses 1129-1130 when dependent, directly or indirectly, on any of clauses 1121-1124, wherein the polymeric layer has a thickness of 0.8 mils+/−10%.

Clause 1132. The overpatch assembly of clause 1131, wherein an adhesive layer between the non-isotropic layer and the polymeric layer has a thickness of one mil+/−10%.

Clause 1133. The overpatch assembly of any of clauses 1056-1132, wherein the on-body device is configured for analyte monitoring.

Clause 1144. A product, comprising: an overpatch assembly according to any of clauses 1056-1133; and a package in which the overpatch assembly is contained, wherein the package comprises a liner surface adhered to an exposed portion of adhesive of the overpatch assembly, wherein the liner surface is secured to the package.

Clause 1145. The product of clause 1144, wherein the package further comprises a conductive element.

Clause 1146. The product of clause 1145, wherein the conductive element is a metallic strip.

Clause 1147. The product of clause 1145, wherein the conductive element is configured to conduct an induced current.

Clause 1148. A product, comprising: an overpatch assembly according to any of clauses 1056-1133; a package in which the overpatch assembly is contained, wherein the package comprises a conductive element.

Clause 1149. The product of clause 1148, wherein the conductive element is secured to the package.

Clause 1150. The product of clause 1148, wherein the conductive element is secured to the overpatch assembly.

Clause 1151. The product of any of clauses 1148-1150, wherein the conductive element is a metallic strip.

Clause 1152. The product of any of clauses 1148-1151, wherein the conductive element is configured to conduct an induced current.

Clause 1153. The product of any of clauses 1148-1152, wherein the package further comprises a liner surface adhered to an exposed portion of adhesive of the overpatch assembly, wherein the liner surface is secured to the package.

Clause 1154. The product of any of clauses 1144-1153, further comprising a kit package comprising: the overpatch assembly, the package having the overpatch assembly therein; and at least one of an on-body device or an inserter for the on-body device.

Clause 1155. A method of testing a kit package, comprising:

applying an electromagnetic field to the kit package, wherein the kit package comprises an overpatch package comprising an overpatch, wherein a conductive element is present in the overpatch package or the overpatch, and wherein the kit package further comprises at least one of an on-body device or an inserter for the on-body device; and sensing a magnitude of an induced electrical current within the kit package.

Clause 1156. The method of clause 1155, further comprising verifying that the overpatch package or the overpatch is present within the kit package.

Clause 1157. The method of clause 1156, wherein the conductive element is configured to conduct an induced current.

Clause 1158. The method of clause 1157, wherein the conductive element is configured as a metallic strip secured to the overpatch package.

Clause 1159. A method of applying an overpatch to a wearer of an on-body device, the method comprising:
positioning an overpatch assembly over at least a portion of the on-body device while a first portion of an adhesive layer of the overpatch is exposed and a second portion of the adhesive layer is unexposed; and
adhering the first portion of the adhesive layer to skin of the wearer while the second portion of the adhesive layer is unexposed.

Clause 1160. The method of clause 1159, wherein adhering the first portion of the adhesive layer to skin of the wearer while the second portion of the adhesive layer is unexposed comprises manually pressing a side of the overpatch assembly opposite to the first portion of the adhesive layer to adhere the first portion of the adhesive layer to the skin.

Clause 1161. The method of any of clauses 1159-1160, further comprising removing, by the user, a first liner to expose the first portion of the adhesive layer prior to positioning the overpatch assembly over the at least a portion of the on-body device.

Clause 1162. The method of clause 1161, wherein the first liner is secured in an interior of a package of the overpatch assembly.

Clause 1163. The method of clause 1162, wherein removing, by the user, the first liner to expose the first portion of the adhesive layer is accomplished by removing the overpatch assembly from within the package.

Clause 1164. The method of any of clauses 1159-1162, further comprising removing at least one folded liner to expose at least a part of the second portion of the adhesive layer after adhering the first portion of the adhesive layer to skin of the wearer.

Clause 1165. The method of clause 1164, further comprising adhering the exposed at least a part of the second portion of the adhesive layer to the skin.

Clause 1166. The method of clause 1165, wherein adhering the exposed at least a part of the second portion of the adhesive layer to the skin comprises manually pressing a side of the overpatch assembly opposite to the at least a part of the second portion of the adhesive layer to adhere the at least a part of the second portion of the adhesive layer to the skin.

Clause 1167. The method of any of clauses 1159-1163, further comprising removing a first folded liner to expose a first section of the second portion of the adhesive layer after adhering the first portion of the adhesive layer to skin of the wearer.

Clause 1168. The method of clause 1167, wherein the overpatch assembly comprises an overpatch having the adhesive layer thereon, wherein the overpatch has an interior space, wherein the first folded liner has an enclosed open region when in an unfolded state, and wherein the first folded liner is configured such that the last portion of a perimeter edge of the enclosed open region to be removed from the adhesive layer is removed at a location of the adhesive layer spaced away from a perimeter of the interior space of the overpatch.

Clause 1169. The method of clause 1167, further comprising adhering the exposed first section of the second portion of the adhesive layer to the skin.

Clause 1170. The method of clause 1169, wherein adhering the exposed first section of the second portion of the adhesive layer to the skin comprises manually pressing a side of the overpatch assembly opposite to the first section of the second portion of the adhesive layer to adhere the first section of the second portion of the adhesive layer to the skin.

Clause 1171. The method of any of clauses 1167-1170, wherein removing the first folded liner comprises concurrently removing and unfolding the first folded liner by pulling on the first folded liner.

Clause 1172. The method of any of clauses 1167-1171, further comprising removing a second folded liner to expose a second section of the second portion of the adhesive layer after adhering the first section of the second portion of the adhesive layer to skin of the wearer.

Clause 1173. The method of clause 1172 when dependent, directly or indirectly, on clause 1168, wherein the second folded liner has an enclosed open region when in an unfolded state, and wherein the second folded liner is configured such that the last portion of a perimeter edge of the enclosed open region to be removed from the adhesive layer is removed at a location of the adhesive layer spaced away from a perimeter of the interior space of the overpatch.

Clause 1174. The method of clause 1172, further comprising adhering the exposed second section of the second portion of the adhesive layer to the skin.

Clause 1175. The method of clause 1174, wherein adhering the exposed second section of the second portion of the adhesive layer to the skin comprises manually pressing a side of the overpatch assembly opposite to the second section of the second portion of the adhesive layer to adhere the second section of the second portion of the adhesive layer to the skin.

Clause 1176. The method of any of clauses 1172-1175, wherein removing the second folded liner comprises concurrently removing and unfolding the second folded liner by pulling on the second secondary adhesive liner.

Clause 1177. The method of any of clauses 1159-1176, wherein the overpatch assembly does not include a removable stiffener layer distinct from the overpatch and the at least one removable folded liner.

Clause 1178. The method of any of clauses 1159-1176, further comprising removing a stiffener layer from the overpatch after fully adhering the overpatch to skin of the wearer of the on-body device.

Clause 1179. The method of any of clauses 1159-1178, wherein the overpatch comprises a backing layer coupled with the adhesive layer.

Clause 1180. The method of any of clauses 1165-1179 when directly or indirectly dependent on clause 1164, further comprising repositioning the overpatch assembly prior to removing the at least one folded liner.

Clause 1181. The method of clause 1180, wherein repositioning the overpatch assembly prior to removing at least one folded liner comprises un-adhering the first portion of the adhesive layer from the skin, moving the overpatch assembly, and then re-adhering the first portion of the adhesive layer to the skin.

Clause 1182. A method of applying an overpatch to a wearer of an on-body device, the method comprising:
positioning a support structure of an overpatch assembly on a housing of the on-body device while a first portion of an adhesive layer of the overpatch is exposed and a second portion of the adhesive layer is unexposed; and
adhering the first portion of the adhesive layer to skin of the wearer while the support structure is on the housing and the second portion of the adhesive layer is unexposed.

Clause 1183. The method of clause 1182, wherein positioning the support structure of the overpatch assembly on the housing of the on-body device comprises positioning the support structure of the overpatch in contact with the housing of the on-body device.

Clause 1184. The method of any of clauses 1182 or 1183, wherein positioning the support structure of the overpatch assembly on the housing of the on-body device comprises aligning the support structure over the housing of the on-body device.

Clause 1185. The method of clause 1184, wherein the housing of the on-body device has a peripheral shape that is not rotationally symmetric, and wherein aligning the support structure over the housing of the on-body device comprises aligning the peripheral shape of the housing of the on-body device with a corresponding peripheral shape of a recess within the support structure.

Clause 1186. The method of clause 1184, wherein aligning the support structure over the housing of the on-body device comprises aligning a physical alignment feature of the housing of the on-body device with a corresponding inverse physical alignment feature of the support structure.

Clause 1187. The method of any of clauses 1184-1186, wherein the on-body device comprises a plurality of side-facing channel terminations or vent outlets and the support structure comprises at least one aperture, wherein aligning the support structure over the housing of the on-body device is such that the plurality of side-facing channel terminations or vent outlets are aligned with the at least one aperture when the support structure is in a final resting position over the on-body device.

Clause 1188. The method of any of clauses 1182-1186, wherein positioning the support structure of the overpatch assembly on the housing of the on-body device comprises sliding the support structure of the overpatch over the housing of the on-body device such that the housing of the on-body device is received within an interior space of the support structure.

Clause 1189. The method of any of clauses 1182-1188, wherein adhering the first portion of the adhesive layer to skin of the wearer while the support structure is on the housing and the second portion of the adhesive layer is unexposed comprises manually pressing a side of the overpatch assembly opposite to the first portion of the adhesive layer to adhere the first portion of the adhesive layer to the skin.

Clause 1190. The method of any of clauses 1182-1189, further comprising removing, by the user, a first skin adhesive liner to expose the first portion of the adhesive layer prior to positioning the support structure of the overpatch assembly on the housing of the on-body device.

Clause 1191. The method of any of clauses 1182-1190, further comprising removing at least one secondary adhesive liner to expose the second portion of the adhesive layer after adhering the first portion of the adhesive layer to skin of the wearer.

Clause 1192. The method of clause 1191, further comprising adhering the exposed second portion of the adhesive layer to the skin.

Clause 1193. The method of clause 1192, wherein adhering the exposed second portion of the adhesive layer to the skin comprises manually pressing a side of the overpatch assembly opposite to the second portion of the adhesive layer to adhere the second portion of the adhesive layer to the skin.

Clause 1194. The method of any of clauses 1182-1190, further comprising removing a first secondary adhesive liner to expose a first section of the second portion of the adhesive layer after adhering the first portion of the adhesive layer to skin of the wearer.

Clause 1195. The method of clause 1194, further comprising adhering the exposed first section of the second portion of the adhesive layer to the skin.

Clause 1196. The method of clause 1195, wherein adhering the exposed first section of the second portion of the adhesive layer to the skin comprises manually pressing a side of the overpatch assembly opposite to the first section of the second portion of the adhesive layer to adhere the first section of the second portion of the adhesive layer to the skin.

Clause 1197. The method of any of clauses 1194-1196, wherein removing the first secondary adhesive liner comprises concurrently removing and unfolding the first secondary adhesive liner by pulling on the first secondary adhesive liner.

Clause 1198. The method of any of clauses 1194-1197, further comprising removing a second secondary adhesive liner to expose a second section of the second portion of the adhesive layer after adhering the first section of the second portion of the adhesive layer to skin of the wearer.

Clause 1199. The method of clause 1198, further comprising adhering the exposed second section of the second portion of the adhesive layer to the skin.

Clause 1200. The method of clause 1199, wherein adhering the exposed second section of the second portion of the adhesive layer to the skin comprises manually pressing a side of the overpatch assembly opposite to the second section of the second portion of the adhesive layer to adhere the second section of the second portion of the adhesive layer to the skin.

Clause 1201. The method of any of clauses 1198-1200, wherein removing the second secondary adhesive liner comprises concurrently removing and unfolding the second secondary adhesive liner by pulling on the second secondary adhesive liner.

Clause 1202. The method of any of clauses 1182-1201, further comprising removing a stiffener unit from the overpatch assembly.

Clause 1203. The method of clause 1202, wherein the stiffener layer is removed by pulling a pull tab of the stiffener unit.

Clause 1204. The method of any of clauses 1202-1203, wherein the stiffener unit comprises an upper liner coupled to a stiffener layer.

Clause 1205. The method of any of clauses 1202-1203, wherein removal of the stiffener unit leaves an overpatch of the overpatch assembly in place on the skin over at least a portion of the on-body device.

Clause 1206. The method of clause 1205, wherein the overpatch comprises a backing layer coupled with the adhesive layer.

Clause 1207. The method of any of clauses 1182-1206, wherein the first portion of the adhesive layer is immediately adjacent the support structure.

Clause 1208. The method of any of clauses 1182-1207, wherein the support structure comprises a first retainer structure, a sidewall, and a second retainer structure.

Clause 1209. The method of clause 1208, wherein the first retainer structure is configured for placement against a first side of the on-body device, and the second retainer structure is configured for placement against skin of the wearer.

Clause 1210. The method of any of clauses 1208 or 1209, wherein the first retainer structure comprises an opening.

Clause 1211. The method of any of clauses 1208-1210, wherein the first retainer structure is configured as a lip.

Clause 1212. The method of any of clauses 1208-1211, wherein positioning the support structure of the overpatch assembly on the housing of the on-body device comprises sliding the support structure of the overpatch over the housing of the on-body device until the housing contacts the upper retainer structure.

Clause 1213. The method of any of clauses 1208-1212, wherein the second retainer structure comprises at least one extension section located adjacent to at least one gap region.

Clause 1214. The method of any of clauses 1208-1212, wherein the first portion of the adhesive layer is in a gap region adjacent an extension section of the support structure.

Clause 1215. The method of any of clauses 1208-1212, wherein the second retainer structure comprises a plurality of extension sections with a plurality of gap regions located therebetween.

Clause 1216. The method of any of clauses 1182-1215, wherein the first portion of the adhesive layer is non-contiguous.

Clause 1217. The method of any of clauses 1182-1216, wherein the first portion of the adhesive layer is adhered to the skin through the plurality of gap regions.

Clause 1218. The method of clause 1190 and/or any of clauses 1191-1217 when directly or indirectly dependent on clause 1190, further comprising attaching the support structure to the first portion of the adhesive layer prior to positioning the support structure of the overpatch assembly on the housing of the on-body device.

Clause 1219. The method of clause 1191 and/or any of clauses 1192-1218 when directly or indirectly dependent on clause 1191, further comprising repositioning the overpatch assembly prior to removing at least one secondary adhesive liner.

Clause 1220. The method of clause 1219, wherein repositioning the overpatch assembly prior to removing at least one secondary adhesive liner comprises un-adhering the first portion of the adhesive layer from the skin, moving the overpatch assembly, and then re-adhering the first portion of the adhesive layer to the skin.

Clause 1221. An adhesive patch for use with an on-body device, comprising: a first adhesive layer; a second adhesive layer; a first structural layer having a first side adhered to the first adhesive layer and a second side adhered to the second adhesive layer; and a second structural layer having a first side adhered to the second adhesive layer.

Clause 1222. The adhesive patch of clause 1221, wherein the first adhesive layer is a skin adhesive layer.

Clause 1223. The adhesive patch of any of clauses 1221-1222, wherein the second adhesive layer adheres the first structural layer to the second structural layer.

Clause 1224. The adhesive patch of any of clauses 1221-1223, wherein the first structural layer comprises a polymeric or plastic material.

Clause 1225. The adhesive patch of any of clauses 1221-1223, wherein the first structural layer comprises a polyester, a polyurethane (PU), a polyethylene, a polyether, an elastane, a polyamide, or a combination of two or more thereof.

Clause 1226. The adhesive patch of any of clauses 1221-1223, wherein the first structural layer comprises a polyethylene terephthalate (PET).

Clause 1227. The adhesive patch of any of clauses 1221-1223, wherein the first structural layer comprises a polyurethane (PU).

Clause 1228. The adhesive patch of any of clauses 1221-1227, wherein the first structural layer is an isotropic layer.

Clause 1229. The adhesive patch of any of clauses 1221-1227, wherein the first structural layer is a non-isotropic layer.

Clause 1230. The adhesive patch of any of clauses 1221-1229, wherein the second structural layer comprises a polymeric or plastic material.

Clause 1231. The adhesive patch of any of clauses 1221-1229, wherein the second structural layer comprises a polyester, a polyurethane (PU), a polyethylene, a polyether, an elastane, a polyamide, or a combination of two or more thereof.

Clause 1232. The adhesive patch of any of clauses 1221-1229, wherein the second structural layer comprises a polyethylene terephthalate (PET).

Clause 1233. The adhesive patch of any of clauses 1221-1229, wherein the second structural layer comprises a polyurethane (PU).

Clause 1234. The adhesive patch of any of clauses 1221-1233, wherein the second structural layer is an isotropic layer.

Clause 1235. The adhesive patch of any of clauses 1221-1233, wherein the second structural layer is a non-isotropic layer.

Clause 1236. The adhesive patch of clause 1221, wherein the first structural layer is a non-isotropic spunlace polymer and the second structural layer comprises polyurethane.

Clause 1237. The adhesive patch of any of clauses 1221-1236, wherein the first adhesive layer comprises a pressure sensitive adhesive.

Clause 1238. The adhesive patch of any of clauses 1221-1237, wherein the first adhesive layer comprises an acrylic adhesive.

Clause 1239. The adhesive patch of any of clauses 1221-1237, wherein the first adhesive layer comprises at least one of a silicone adhesive, a hydrocarbon adhesive, a polyurethane adhesive, a natural rubber adhesive, and/or a synthetic rubber adhesive.

Clause 1240. The adhesive patch of any of clauses 1221-1239, wherein the second adhesive layer comprises a pressure sensitive adhesive.

Clause 1241. The adhesive patch of any of clauses 1221-1240, wherein the second adhesive layer comprises an acrylic adhesive.

Clause 1242. The adhesive patch of any of clauses 1221-1240, wherein the second adhesive layer comprises at least one of a silicone adhesive, a hydrocarbon adhesive, a polyurethane adhesive, a natural rubber adhesive, and/or a synthetic rubber adhesive.

Clause 1243. The adhesive patch of any of clauses 1221-1242, wherein the patch exhibits a moisture vapor transmission rate (MVTR) of 115 grams/square meter/day or higher.

Clause 1244. The adhesive patch of any of clauses 1221-1242, wherein the patch exhibits a moisture vapor transmission rate (MVTR) of 175 grams/square meter/day or higher.

Clause 1245. The adhesive patch of any of clauses 1221-1242, wherein the patch exhibits a moisture vapor transmission rate (MVTR) of 200 grams/square meter/day or higher.

Clause 1246. The adhesive patch of any of clauses 1221-1245, wherein the first structural layer has a thickness of 11 mils+/−25%.

Clause 1247. The adhesive patch of any of clauses 1221-1246, wherein the first adhesive layer has a thickness of 4 mils+/−25%.

Clause 1248. The adhesive patch of any of clauses 1221-1247, wherein the second structural layer has a thickness of 0.8 mils+/−25%.

Clause 1249. The adhesive patch of any of clauses 1221-1248, wherein the second adhesive layer has a thickness of one mil+/−25%.

Clause 1250. The adhesive patch of any of clauses 1221-1245, wherein the first structural layer has a thickness of 11 mils+/−10%.

Clause 1251. The adhesive patch of any of clauses 1221-1245 and 1250, wherein the first adhesive layer has a thickness of 4 mils+/−10%.

Clause 1252. The adhesive patch of any of clauses 1221-1245 and 1251, wherein the second structural layer has a thickness of 0.8 mils+/−10%.

Clause 1253. The adhesive patch of any of clauses 1221-1245 and 1252, wherein the second adhesive layer has a thickness of one mil+/−10%.

Clause 1254. The adhesive patch of any of clauses 1221-1253, further comprising a third adhesive layer, wherein the third adhesive layer adheres the second structural layer to a housing of an on-body device.

Clause 1255. The adhesive patch of clause 1254, wherein the third adhesive layer comprises a pressure sensitive adhesive.

Clause 1256. The adhesive patch of clause 1254, wherein the third adhesive layer comprises an acrylic adhesive.

Clause 1257. The adhesive patch of clause 1254, wherein the third adhesive layer comprises at least one of a silicone adhesive, a hydrocarbon adhesive, a polyurethane adhesive, a natural rubber adhesive, and/or a synthetic rubber adhesive.

Clause 1258. The adhesive patch of any of clauses 1221-1257, wherein the adhesive patch is configured as a main adhesive patch for an on-body device comprising a housing.

Clause 1259. The adhesive patch of any of clauses 1221-1254, wherein the adhesive patch is configured as an overpatch for at least a portion of an on-body device comprising a housing and a main adhesive patch.

Clause 1260. An on-body device configured to monitor an analyte of a body, comprising: an on-body device housing comprising electronics configured to interface with an analyte sensor; and an adhesive patch adhered to the on-body device housing, wherein the adhesive patch is configured in accordance with the adhesive patch of any of clauses 1221-1259.

Clause 1261. An analyte monitoring system, comprising: an on-body device comprising a housing comprising electronics configured to interface with an analyte sensor and a first adhesive patch adhered to the on-body device housing; and a second adhesive patch configured to be applied over the housing and the first adhesive patch, wherein the first adhesive patch is configured in accordance with any of clauses 1221-1259.

Clause 1262. A method of applying an on-body device to a body, comprising: applying an adhesive patch to skin of the body, wherein the adhesive patch is coupled to an on-body device housing, wherein the adhesive patch is configured in accordance with the adhesive patch of any of clauses 1221-1259.

Clause 1263. The adhesive patch of any of clauses 1015-1046 in combination with any one or more the on-body devices and features thereof of any of clauses 1 to 154 or 178 to 288 or 322 to 323.

Clause 1264. The adhesive patch of any of clauses 1015-1046 in combination with any of the reinforcement devices and one or more features thereof of any of clauses 840-986.

Clause 1265. The adhesive patch of any of clauses 1015-1046 in combination with the features of the adhesive patches of any of clauses 654-812.

Clause 1266. The overpatch assembly of any of clauses 1056-1133 in combination with any one or more of the on-body devices and features thereof of any of clauses 1 to 154 or 178 to 288 or 322 to 323.

Clause 1267. The overpatch assembly of any of clauses 1015-1046 in combination with any of the reinforcement devices and one or more features thereof of any of clauses 840-986.

Clause 1268. The overpatch assembly of any of clauses 1015-1046 in combination with the features of the adhesive patches of any of clauses 654-812.

Clause 1269. The adhesive patch of any of clauses 1221-1259 in combination with any one or more the on-body devices and features thereof of any of clauses 1 to 154 or 178 to 288 or 322 to 323.

Clause 1270. The adhesive patch of any of clauses 1221-1259 in combination with any of the reinforcement devices and one or more features thereof of any of clauses 840-986.

Clause 1271. The adhesive patch of any of clauses 1221-1259 in combination with the features of the adhesive patches of any of clauses 654-812.

Clause 1272. An on-body device for monitoring an analyte level of a user, comprising: a housing; sensor electronics, wherein the housing is configured to retain the sensor electronics; a sensor; and an adhesive patch attached to the housing and configured to attach the housing to skin of the user.

Clause 1273. The on-body device of any of clauses 1 to 154, 178 to 288, 585 to 589, 1009, or 1179, or the on-body device patch of clause 839, or any preceding clause when directly or indirectly dependent on any of clauses 1 to 154, 178 to 288, or 585 to 589, 839, 1009, or 1272 wherein:
the housing comprises a first part and a second part attached together, wherein the housing is water-resistant;
the electronics comprise:
   processing electronics present on a semiconductor chip; and
   wireless communications electronics present on the semiconductor chip and configured to communicate according to a Bluetooth Low Energy protocol;
the sensor has a flexible substrate, the sensor comprising:
   a first portion retained within the housing and comprising three contacts configured to be in electrical contact with the sensor electronics; and
   a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion.

Clause 1274. The on-body device of clause 1273, further comprising a temperature sensor present on a side of the first part of the housing adhered to the adhesive patch and wherein:
the sensor electronics are coupled to a printed circuit board, the sensor electronics further comprising a coin cell battery and a magnetic switch;
the sensor comprises a fold; and
the adhesive patch protrudes beyond a peripheral boundary of the housing.

Clause 1275. The on-body device of any of clauses 1179-1181, further comprising the reinforcement device of any one of clauses 840-913, 917, 918, 921 to 986, 992, 993, or 996 to 1000, or the reinforcement assembly of clause 1001.

Clause 1276. The on-body device of any of clauses 1179-1182, wherein the adhesive patch is the adhesive patch of any of clauses 1010-1013, 1015-1046, and/or 1221-1259.

Clause 1277. An analyte monitoring system, comprising:
an on-body device comprising a sensor; and
an inserter comprising:
   a removable cap;
   a sharp;
   a retraction spring;
   a housing;
   a handle configured to move from an initial position to an advanced position with respect to the housing to advance the sharp into skin of a recipient's body and concurrently advance the on-body device into position on the recipient's body,
wherein the inserter is configured to automatically retract the sharp with the retraction spring.

Clause 1278. The analyte monitoring system of clause 1277, wherein the on-body device is housed within an interior space of the inserter, the interior space being sealed by a removable cap coupled with the handle and/or housing of the inserter, and wherein the sharp and the sensor are maintained in a sterile environment by a sheath of the removable cap.

Clause 1279. The analyte monitoring system of any of clauses 1277 or 1278, wherein:
the sharp comprises a distal tip portion with a V-shaped portion having a first angle of 20 to 60 degrees;
the sharp comprises a proximal shaft portion comprising a first sidewall, a floor, and a second sidewall together providing a U-shape or a C-shape cross-sectional profile; and
the sharp comprises a transition portion between the distal tip portion and the proximal shaft portion where the first sidewall and the second sidewall each have a height from the floor that is sloped at a second angle of 7 to 35 degrees.

Clause 1280. The analyte monitoring system of clause 1279, wherein the first angle is between 45 and 55 degrees and the second angle is between 20 and 30 degrees.

Clause 1281. The analyte monitoring system of any of clauses 1277 to 1280, further comprising:
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1282. An analyte monitoring system, comprising:
an on-body device comprising a sensor; and
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1283. The analyte monitoring system of any of clauses 1277-1282, further comprising an overpatch.

Clause 1284. The analyte monitoring system of clause 1190, wherein the overpatch is the overpatch of any of clauses 654-812, 1015-1046, 1056-1133, and/or 1221-1259.

Clause 1285. The analyte monitoring system of any of clauses 1277-1284, wherein the on-body device is the on-body device of any of clauses 1 to 154, 178 to 288, 585 to 589, 839, 1009, or 1272 to 1276, or any preceding clause when directly or indirectly dependent on any of clauses 1 to 154, 178 to 288, 585 to 589, 839, 1009, or 1272 to 1276.

Clause 1286. The on-body device of any of clauses 1 to 154, 178 to 288, 585 to 589, 1009, or 1272, or the on-body device patch of clause 839, or any preceding clause when directly or indirectly dependent on any of clauses 1 to 154, 178 to 288, or 585 to 589, 839, 1009, or 1272, wherein:
the housing is configured to be water-resistant;
the electronics comprise:
   sensor interface electronics;
   wireless communications electronics configured to communicate according to a Bluetooth Low Energy protocol; and
the sensor is configured to detect or measure an analyte with a working electrode and a counter/reference electrode, the sensor comprising:
   a central substrate configured as a wire; and
   a plurality of layers on the central substrate, the plurality of layers comprising a diffusion limiting layer, an enzyme layer, and an interferent layer.

Clause 1287. The on-body device of clause 1286, wherein the electronics further comprise:
a first antenna configured for communication according to the Bluetooth Low Energy protocol;
a second antenna configured for communication according to a Near Field Communication protocol;
a coin cell battery; and
a magnetic switch.

Clause 1288. The on-body device of any of clauses 1286 or 1287, wherein the sensor is in a bent configuration, wherein the housing is a molded housing comprising a plurality of first recesses configured to engagement with a plurality of first projections of an inserter to hold the on-body device to the inserter and comprising a second recess configured to interface with a second projection of the inserter to inhibit rotation of the on-body device, and wherein the adhesive patch protrudes beyond a peripheral boundary of the housing.

Clause 1289. The on-body device of any of clauses 1286 to 1288, further comprising the reinforcement device of any one of clauses 840-913, 917, 918, 921 to 986, 992, 993, or 996 to 1000, or the reinforcement assembly of clause 1001.

Clause 1290. The on-body device of any of clauses 1286-1289, wherein the adhesive patch is the adhesive patch of any of clauses 1010-1013, 1015-1046, and/or 1221-1259.

Clause 1291. An analyte monitoring system, comprising:
an on-body device comprising a sensor; and
an inserter configured to automatically advance a sharp and move the on-body device and configured to automatically retract the sharp, the inserter comprising:
   a removable cap;
   the sharp;
   a first spring configured to advance the sharp;
   a second spring configured to retract the sharp;
   a housing comprising an actuator configured to initiate advancement of the sharp.

Clause 1292. The analyte monitoring system of clause 1291, wherein the on-body device is housed within an interior space of the inserter, the interior space being sealed by the removable cap.

Clause 1293. The analyte monitoring system of any of clauses 1291 or 1292, wherein:

the sharp comprises a shaft comprising a first sidewall, a floor, and a second sidewall together providing a U-shape or a C-shape cross-sectional profile, the shaft having a beveled distal tip having an angle of 20 to 45 degrees.

Clause 1294. The analyte monitoring system of any of clauses 1291 to 1293, further comprising:
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1295. An analyte monitoring system, comprising:
an on-body device comprising a sensor; and
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1296. The analyte monitoring system of any of clauses 1291 to 1295, further comprising an overpatch.

Clause 1297. The analyte monitoring system of clause 1296, wherein the overpatch is the overpatch of any of clauses 654-812, 1015-1046, 1056-1133, and/or 1259.

Clause 1298. The analyte monitoring system of any of clauses 1198 to 1204, wherein the on-body device is the on-body device of any of clauses 1 to 154, 178 to 288, 585 to 589, 839, 1009, 1272 to 1276, or 1286 to 1290, or any preceding clause when directly or indirectly dependent on any of clauses 1 to 154, 178 to 288, 585 to 589, 839, 1009, 1272 to 1276, or 1286 to 1290.

Clause 1299. The on-body device of any of clauses 1 to 154, 178 to 288, 585 to 589, 1009, or 1272, or the on-body device patch of clause 839, or any preceding clause when directly or indirectly dependent on any of clauses 1 to 154, 178 to 288, or 585 to 589, 839, 1009, or 1272, wherein:
the housing comprises a first part and a second part attached together, wherein the housing is water-resistant;
the electronics comprise:
   processing electronics present on a semiconductor chip; and
   wireless communications electronics present on the semiconductor chip and configured to communicate according to a Bluetooth Low Energy protocol; and
the sensor has a flexible substrate, the sensor comprising:
   a first portion retained within the housing and comprising three contacts configured to be in electrical contact with the sensor electronics; and
   a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion.

Clause 1300. The on-body device of clause 1299, wherein:
the electronics further comprise a coin cell battery, a magnetic switch, a printed circuit board, and an antenna,
wherein the antenna is configured to communicate according to the Bluetooth Low Energy protocol, and wherein the antenna is mounted on the housing and is electrically connected to the wireless communications electronics on the printed circuit board;
the housing comprises a plurality of recesses adapted to interface with inserter projections; and
the adhesive patch protrudes beyond a peripheral boundary of the housing.

Clause 1301. The on-body device of clause 1300, wherein the coin cell battery is electrically connected to the printed circuit board and is held adjacent to the printed circuit board such that the coin cell battery and the printed circuit board are both in a same plane.

Clause 1302. The on-body device of any of clauses 1299 to 1301, further comprising the reinforcement device of any one of clauses 840-913, 917, 918, 921 to 986, 992, 993, or 996 to 1000, or the reinforcement assembly of clause 1001.

Clause 1303. The on-body device of any of clauses 1299 to 1302, wherein the adhesive patch is the adhesive patch of any of clauses 1010-1013, 1015-1046, and/or 1221-1259.

Clause 1304. An analyte monitoring system, comprising:
an on-body device comprising a sensor; and
an inserter configured to automatically advance a sharp, move the on-body device, and automatically retract the sharp, the inserter comprising:
   a removable cap;
   the sharp;
   a first spring configured to advance the sharp;
   a second spring configured to retract the sharp; and
   a housing comprising an actuator configured to initiate advancement of the sharp.

Clause 1305. The analyte monitoring system of clause 1304, wherein the on-body device is housed within an interior space of the inserter, the interior space being sealed by a removable cap coupled with the housing of the inserter, and wherein the sharp and the sensor are maintained in a sterile environment by a sheath of the removable cap.

Clause 1306. The analyte monitoring system of any of clauses 1304 or 1305, wherein:
the sharp comprises a distal tip portion with a V-shaped portion having a first angle of 20 to 60 degrees;
the sharp comprises a proximal shaft portion comprising a first sidewall, a floor, and a second sidewall together providing a U-shape or a C-shape cross-sectional profile; and
the sharp comprises a transition portion between the distal tip portion and the proximal shaft portion where the first sidewall and the second sidewall each have a height from the floor that is sloped at a second angle of 7 to 35 degrees.

Clause 1307. The analyte monitoring system of clause 1306, wherein the first angle is between 28 and 38 degrees and the second angle is between 18 and 28 degrees.

Clause 1308. The analyte monitoring system of any of clauses 1304 to 1307, further comprising:
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1309. An analyte monitoring system, comprising:
an on-body device comprising a sensor; and
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1310. The analyte monitoring system of any of clauses 1304 to 1309, further comprising an overpatch.

Clause 1311. The analyte monitoring system of clause 1310, wherein the overpatch is the overpatch of any of clauses 654-812, 1015-1046, 1056-1133, and/or 1259.

Clause 1312. The analyte monitoring system of any of clauses 1304 to 1311, wherein the on-body device is the on-body device of any of clauses 1 to 154, 178 to 288, 585 to 589, 839, 1009, 1272 to 1276, 1286 to 1290, or 1299 to 1303, or any preceding clause when directly or indirectly dependent on any of clauses 1 to 154, 178 to 288, 585 to 589, 839, 1009, 1272 to 1276, 1286 to 1290, or 1299 to 1303.

Clause 1313. The on-body device of any of clauses 1 to 154, 178 to 288, 585 to 589, 1009, or 1272, or the on-body device patch of clause 839, or any preceding clause when directly or indirectly dependent on any of clauses 1 to 154, 178 to 288, or 585 to 589, 839, 1009, or 1272, wherein the on-body device comprises a sensor connector, and wherein:
the housing comprises a first part and a second part attached together;
the electronics comprise:
   processing electronics present on a semiconductor chip; and
   wireless communications electronics present on the semiconductor chip and configured to communicate according to a Bluetooth Low Energy protocol;
the sensor has a flexible substrate, the sensor coupled with the sensor connector and comprising:
   a first portion retained within the housing and comprising three contacts configured to be in electrical contact with the sensor electronics; and
   a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion, and
the housing comprises a recess configured to receive the sensor connector, and wherein the housing is configured to be water-resistant when the sensor connector is received within the recess.

Clause 1314. The on-body device of clause 1313, wherein:
the electronics further comprise a coin cell battery, a magnetic switch, a printed circuit board, and an antenna,
wherein the antenna is configured to communicate according to the Bluetooth Low Energy protocol;
the housing comprises a plurality of recesses adapted to interface with inserter projections; and
the adhesive patch protrudes beyond a peripheral boundary of the housing.

Clause 1315. The on-body device of clause 1314, wherein the coin cell battery is electrically connected to the printed circuit board and is held adjacent to the printed circuit board such that the coin cell battery and the printed circuit board are both in a same plane.

Clause 1316. The on-body device of any of clauses 1313 to 1315, further comprising the reinforcement device of any one of clauses 840-913, 917, 918, 921 to 986, 992, 993, or 996 to 1000, or the reinforcement assembly of clause 1001.

Clause 1317. The on-body device of any of clauses 1313 to 1316, wherein the adhesive patch is the adhesive patch of any of clauses 1010-1013, 1015-1046, and/or 1221-1259.

Clause 1318. An analyte monitoring system, comprising:
an on-body device comprising a recess; and
an inserter configured to hold the on-body device and automatically advance a sensor connector and a sharp into the recess of the on-body device, wherein the sensor connector is coupled with a sensor, wherein the inserter is configured to automatically retract the sharp, the inserter comprising:
   the sharp;
   a first spring configured to advance the sharp;
   a second spring configured to retract the sharp; and
   a housing comprising an actuator configured to initiate advancement of the sharp.

Clause 1319. The analyte monitoring system of clause 1318, wherein:
the sharp comprises a distal tip portion with a V-shaped portion having a first angle of 20 to 60 degrees;
the sharp comprises a proximal shaft portion comprising a first sidewall, a floor, and a second sidewall together providing a U-shape or a C-shape cross-sectional profile; and
the sharp comprises a transition portion between the distal tip portion and the proximal shaft portion where the first sidewall and the second sidewall each have a height from the floor that is sloped at a second angle of 7 to 35 degrees.

Clause 1320. The analyte monitoring system of clause 1319, wherein the first angle is between 30 and 40 degrees and the second angle is between 13 and 23 degrees.

Clause 1321. The analyte monitoring system of any of clauses 1318 to 1320, further comprising:
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1322. An analyte monitoring system, comprising:
an on-body device comprising a sensor; and
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1323. The analyte monitoring system of any of clauses 1225 to 1230, further comprising an overpatch.

Clause 1324. The analyte monitoring system of clause 1231, wherein the overpatch is the overpatch of any of clauses 654-812, 1015-1046, 1056-1133, and/or 1259.

Clause 1325. The analyte monitoring system of any of clauses 1318 to 1324, wherein the on-body device is the on-body device of any of clauses 1 to 154, 178 to 288, 585 to 589, 839, 1009, 1272 to 1276, 1286 to 1290, 1299 to 1303, or 1313 to 1317, or any preceding clause when directly or indirectly dependent on any of clauses 1 to 154, 178 to 288, 585 to 589, 839, 1009, 1272 to 1276, 1286 to 1290, 1299 to 1303, or 1313 to 1317.

Clause 1326. The on-body device of any of clauses 1 to 154, 178 to 288, 585 to 589, 1009, or 1272, or the on-body device patch of clause 839, or any preceding clause when directly or indirectly dependent on any of clauses 1 to 154, 178 to 288, or 585 to 589, 839, 1009, or 1272, wherein the on-body device comprises a sensor connector, and wherein:
the housing comprises a first part and a second part attached together;
the electronics comprise:
   processing electronics present on a semiconductor chip; and
   wireless communications electronics present on the semiconductor chip and configured to communicate according to a Bluetooth Low Energy protocol;
the sensor has a flexible substrate, the sensor coupled with the sensor connector and comprising:
   a first portion retained within the housing and comprising three contacts configured to be in electrical contact with the sensor electronics; and
   a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion; and
the housing is configured to be water-resistant.

Clause 1327. The on-body device of clause 1326, wherein:
the electronics further comprise a coin cell battery, a magnetic switch, a printed circuit board, and an antenna,
wherein the antenna is configured to communicate according to the Bluetooth Low Energy protocol;
the housing comprises a recess adapted to interface with an inserter projection;
the adhesive patch protrudes beyond a peripheral boundary of the housing;
the sensor comprises a fold; and
the adhesive patch is attached to the first part of the housing.

Clause 1328. The on-body device of clause 1327, wherein the coin cell battery is electrically connected to the printed circuit board and is held adjacent to the printed circuit board such that the coin cell battery and the printed circuit board are both in a same plane.

Clause 1329. The on-body device of any of clauses 1326 to 1328, further comprising the reinforcement device of any one of clauses 840-913, 917, 918, 921 to 986, 992, 993, or 996 to 1000, or the reinforcement assembly of clause 1001.

Clause 1330. The on-body device of any of clauses 1326-1329, wherein the adhesive patch is the adhesive patch of any of clauses 1010-1013, 1015-1046, and/or 1221-1259.

Clause 1331. An analyte monitoring system, comprising:
an on-body device comprising a sensor; and
an inserter configured to automatically advance a sharp, move the on-body device, and automatically retract the sharp, the inserter comprising:
   a removable cap;
   the sharp;
   a first spring configured to advance the sharp;
   a second spring configured to retract the sharp; and
   a housing comprising an actuator configured to initiate advancement of the sharp.

Clause 1332. The analyte monitoring system of clause 1331, wherein the on-body device is housed within an interior space of the inserter, the interior space being sealed by the removable cap coupled with the housing of the inserter, and wherein the sharp and the sensor are maintained in a sterile environment by a sheath of the removable cap.

Clause 1333. The analyte monitoring system of any of clauses 1331 or 1332, wherein:
the sharp comprises a distal tip portion with a V-shaped portion having a first angle of 20 to 60 degrees;
the sharp comprises a proximal shaft portion comprising a first sidewall, a floor, and a second sidewall together providing a U-shape or a C-shape cross-sectional profile; and
the sharp comprises a transition portion between the distal tip portion and the proximal shaft portion where the first sidewall and the second sidewall each have a height from the floor that is sloped at a second angle of 7 to 35 degrees.

Clause 1334. The analyte monitoring system of clause 1333, wherein the first angle is between 31 and 41 degrees and the second angle is between 13 and 23 degrees.

Clause 1335. The analyte monitoring system of any of clauses 1331 to 1334, further comprising:
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1336. An analyte monitoring system, comprising:
an on-body device comprising a sensor; and
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1337. The analyte monitoring system of any of clauses 1331 to 1336, further comprising an overpatch.

Clause 1338. The analyte monitoring system of clause 1337, wherein the overpatch is the overpatch of any of clauses 654-812, 1015-1046, 1056-1133, and/or 1259.

Clause 1339. The analyte monitoring system of any of clauses 1331 to 1338, wherein the on-body device is the on-body device of any of clauses 1 to 154, 178 to 288, 585 to 589, 839, 1009, 1272 to 1276, 1286 to 1290, 1299 to 1303, 1313 to 1317, or 1326 to 1330, or any preceding clause when directly or indirectly dependent on any of clauses 1 to 154, 178 to 288, 585 to 589, 839, 1009, 1272 to 1276, 1286 to 1290, 1299 to 1303, 1313 to 1317, or 1326 to 1330.

Clause 1340. An on-body device of any of clauses 1 to 154, 178 to 288, 585 to 589, 1009, or 1272, or the on-body device patch of clause 839, or any preceding clause when directly or indirectly dependent on any of clauses 1 to 154, 178 to 288, or 585 to 589, 839, 1009, or 1272, wherein the on-body device comprises a sensor connector, and wherein:
the housing comprising a first part and a second part attached together;
the electronics comprise:
   processing electronics present on a semiconductor chip; and
   wireless communications electronics present on the semiconductor chip and configured to communicate according to a Bluetooth Low Energy protocol;
the sensor has a flexible substrate, the sensor coupled with the sensor connector and comprising:
   a first portion retained within the housing and comprising three contacts configured to be in electrical contact with the sensor electronics; and
   a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion; and
the housing comprises a recess configured to receive the sensor connector, and wherein the housing is configured to be water-resistant when the sensor connector is received within the recess.

Clause 1341. The on-body device of clause 1340, wherein:
the electronics further comprise a coin cell battery, a magnetic switch, a printed circuit board, and an antenna,
wherein the antenna is configured to communicate according to the Bluetooth Low Energy protocol;
the housing comprises a recess adapted to interface with an inserter projection;
the adhesive patch protrudes beyond a peripheral boundary of the housing;
the sensor comprises a fold; and
the adhesive patch is attached to the first part of the housing.

Clause 1342. The on-body device of clause 1340 or 1341, further comprising the reinforcement device of any one of clauses 840-913, 917, 918, 921 to 986, 992, 993, or 996 to 1000, or the reinforcement assembly of clause 1001.

Clause 1343. The on-body device of any of clauses 1248 to 1250, wherein the adhesive patch is the adhesive patch of any of clauses 1010-1013, 1015-1046, and/or 1221-1259.

Clause 1344. An analyte monitoring system, comprising:
an on-body device comprising a sensor; and
an inserter configured to automatically advance a sharp, move the on-body device, and automatically retract the sharp, the inserter comprising:
   a removable cap;
   the sharp;
   a first spring configured to advance the sharp;
   a second spring configured to retract the sharp; and
   a housing comprising an actuator configured to initiate advancement of the sharp.

Clause 1345. The analyte monitoring system of clause 1344, wherein:
the sharp comprises a distal tip portion with a V-shaped portion having a first angle of 20 to 60 degrees;
the sharp comprises a proximal shaft portion comprising a first sidewall, a floor, and a second sidewall together providing a U-shape or a C-shape cross-sectional profile; and
the sharp comprises a transition portion between the distal tip portion and the proximal shaft portion where the first sidewall and the second sidewall each have a height from the floor that is sloped at a second angle of 7 to 35 degrees.

Clause 1346. The analyte monitoring system of clause 1345, wherein the first angle is between 20 and 30 degrees and the second angle is between 8 and 18 degrees.

Clause 1347. The analyte monitoring system of any of clauses 1344 to 1346, further comprising:
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1348. An analyte monitoring system, comprising:
an on-body device comprising a sensor; and
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1349. The analyte monitoring system of any of clauses 1344 to 1348, further comprising an overpatch.

Clause 1350. The analyte monitoring system of clause 1349, wherein the overpatch is the overpatch of any of clauses 654-812, 1015-1046, 1056-1133, and/or 1259.

Clause 1351. The analyte monitoring system of any of clauses 1344 to 1350, wherein the on-body device is the on-body device of any of clauses 1 to 154, 178 to 288, 585 to 589, 839, 1009, 1272 to 1276, 1286 to 1290, 1299 to 1303, 1313 to 1317, 1326 to 1330, or 1340 to 1343, or any preceding clause when directly or indirectly dependent on any of clauses 1 to 154, 178 to 288, 585 to 589, 839, 1009, 1272 to 1276, 1286 to 1290, 1299 to 1303, 1313 to 1317, 1326 to 1330, or 1340 to 1343.

Clause 1352. An on-body device of any of clauses 1 to 154, 178 to 288, 585 to 589, 1009, or 1272, or the on-body device patch of clause 839, or any preceding clause when directly or indirectly dependent on any of clauses 1 to 154, 178 to 288, or 585 to 589, 839, 1009, or 1272, wherein the on-body device comprises a sensor connector, and wherein:
the housing comprises a first part and a second part attached together;
the electronics comprise:
   processing electronics present on a semiconductor chip; and
   wireless communications electronics configured to communicate according to a Near Field Communication protocol and a Bluetooth Low Energy protocol, wherein at least a portion of the wireless communications electronics configured to communicate according to the Bluetooth Low Energy protocol are present on the semiconductor chip;
the sensor has a flexible substrate, the sensor coupled with the sensor connector and comprising:
   a first portion retained within the housing and comprising three contacts configured to be in electrical contact with the sensor electronics; and
   a second portion configured to be positioned in the body of the user for monitoring a level of an analyte of the user, wherein the first portion has a length and a width arranged in a plane and a thickness perpendicular to the plane, wherein the thickness is smaller than the width, and wherein the second portion comprises three electrodes connected to connections that are connected to the three contacts of the first portion; and
the housing is configured to be water-resistant.

Clause 1353. The on-body device of clause 1352, wherein:
the electronics further comprise a coin cell battery, a magnetic switch, a printed circuit board, a first antenna, and a second antenna;
wherein the first antenna is configured to communicate according to the Bluetooth Low Energy protocol and the second antenna is configured to communicate according to the Near Field Communication protocol;
the housing comprises a recess adapted to interface with an inserter projection;
the adhesive patch protrudes beyond a peripheral boundary of the housing; and
the adhesive patch is attached to the first part of the housing.

Clause 1354. The on-body device of clause 1353, wherein the coin cell battery is electrically connected to the printed circuit board and is held adjacent to the printed circuit board such that the coin cell battery and the printed circuit board are both in a same plane.

Clause 1355. The on-body device of any of clauses 1352 to 1354, further comprising the reinforcement device of any one of clauses 840-913, 917, 918, 921 to 986, 992, 993, or 996 to 1000, or the reinforcement assembly of clause 1001.

Clause 1356. The on-body device of any of clauses 1352 to 1355, wherein the adhesive patch is the adhesive patch of any of clauses 1010-1013, 1015-1046, and/or 1221-1259.

Clause 1357. An analyte monitoring system, comprising:
an on-body device comprising a sensor; and
an inserter configured to automatically advance a sharp, move the on-body device, and automatically retract the sharp, the inserter comprising:
   a removable cap;
   the sharp;
   a first spring configured to advance the sharp;
   a second spring configured to retract the sharp; and
   a housing comprising an actuator configured to initiate advancement of the sharp.

Clause 1358. The analyte monitoring system of clause 1357, wherein the on-body device is housed within an interior space of the inserter, the interior space being sealed by the removable cap coupled with the housing of the inserter, and wherein the sharp and the sensor are maintained in a sterile environment by a sheath of the removable cap.

Clause 1359. The analyte monitoring system of any of clauses 1357 or 1358, wherein:
the sharp comprises a distal tip portion with a V-shaped portion having a first angle of 20 to 60 degrees;
the sharp comprises a proximal shaft portion comprising a first sidewall, a floor, and a second sidewall together providing a U-shape or a C-shape cross-sectional profile; and
the sharp comprises a transition portion between the distal tip portion and the proximal shaft portion where the first sidewall and the second sidewall each have a height from the floor that is sloped at a second angle of 7 to 35 degrees.

Clause 1360. The analyte monitoring system of clause 1359, wherein the first angle is between 27 and 37 degrees and the second angle is between 7 and 17 degrees.

Clause 1361. The analyte monitoring system of any of clauses 1357 to 1360, further comprising:
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1362. An analyte monitoring system, comprising:
an on-body device comprising a sensor; and
a receiver unit comprising a software application for execution on the receiver unit and a display, wherein the receiver unit is configured to receive analyte data associated with the analyte level of the user from the on-body device and to display information corresponding to the analyte data to the user,
wherein the software application is configured to generate a present analyte value of the user, based on a measurement by the sensor, for output to the user not before expiration of a warm-up period of the sensor, and
wherein the receiver unit and/or the on-body device are configured to apply a factory calibration to, in part, calculate an analyte value for display to the user.

Clause 1363. The analyte monitoring system of any of clauses 1357 to 1362, further comprising an overpatch.

Clause 1364. The analyte monitoring system of clause 1363, wherein the overpatch is the overpatch of any of clauses 654-812, 1015-1046, 1056-1133, and/or 1259.

Clause 1365. The analyte monitoring system of any of clauses 1357 to 1364, wherein the on-body device is the on-body device of any of clauses 1 to 154, 178 to 288, 585 to 589, 839, 1009, 1272 to 1276, 1286 to 1290, 1299 to 1303, 1313 to 1317, 1326 to 1330, 1340 to 1343, or 1352 to 1356, or any preceding clause when directly or indirectly dependent on any of clauses 1 to 154, 178 to 288, 585 to 589, 839, 1009, 1272 to 1276, 1286 to 1290, 1299 to 1303, 1313 to 1317, 1326 to 1330, 1340 to 1343, or 1352 to 1356.

Clause 1366. The analyte monitoring system of any of clauses 1277 to 1285, 1291 to 1298, 1304 to 1312, 1318 to 1325, 1331 to 1339, 1344 to 1351, or 1357 to 1365, wherein the on-body device has a designed wear duration that comprises 14 days.

Clause 1367. The analyte monitoring system of any of clauses 1277 to 1285, 1291 to 1298, 1304 to 1312, 1318 to 1325, 1331 to 1339, 1344 to 1351, or 1357 to 1365, wherein the on-body device has a designed wear duration of 15 to 30 days.

Clause 1368. The analyte monitoring system of any of clauses 1277 to 1285, 1291 to 1298, 1304 to 1312, 1318 to 1325, 1331 to 1339, 1344 to 1351, or 1357 to 1365, wherein the on-body device has a designed wear duration of 31 to 45 days.

Clause 1369. The analyte monitoring system of any of clauses 1277 to 1285, 1291 to 1298, 1304 to 1312, 1318 to 1325, 1331 to 1339, 1344 to 1351, or 1357 to 1365, wherein the on-body device has a designed wear duration that is 45 days or greater.

Clause 1370. The on-body device of any of clauses 1 to 154, 178 to 288, 322 to 323, or 324 to 610, wherein the on-body device comprises an injection molded housing.

Clause 1371. The on-body device of any of clauses 1 to 154, 178 to 288, 322 to 323, or 324 to 610, wherein the on-body device comprises a one-shot molded housing.

Clause 1372. The on-body device of any of clauses 1 to 154, 178 to 288, 322 to 323, or 324 to 610, wherein the on-body device comprises an over-molded housing.

Clause 1373. The on-body device of any of clauses 1 to 154, 178 to 288, 322 to 323, or 324 to 610, wherein the on-body device comprises a housing with at least two parts.

Clause 1374. The on-body device of any of clauses 1 to 154, 178 to 288, 322 to 323, 324 to 610, or 1370 to 1373, wherein the on-body device comprises a housing comprising a first part and a second part assembled together with sensor electronics arranged between.

Clause 1375. The on-body device of any of clauses 1 to 154, 178 to 288, 322 to 323, 324 to 610, or 1370 to 1373, wherein the on-body device comprises a housing with a recess configured to receive a sensor connector, wherein the recess is arranged in a top of the housing.

Clause 1376. The on-body device of any of clauses 1 to 154, 178 to 288, 322 to 323, 324 to 610, or 1370 to 1373, wherein the on-body device comprises a housing with a recess configured to receive a sensor connector, wherein the recess is arranged in a bottom of the housing.

Clause 1377. The on-body device of any of clauses 1375 or 1376, wherein the sensor connector is coupled with an analyte sensor.

Clause 1378. The on-body device of any of clauses 1 to 154, 178 to 288, 322 to 323, 324 to 610, or 1370 to 1373, wherein the on-body device comprises a housing with a recess configured to receive an analyte sensor, wherein the recess is arranged in a bottom of the housing.

Clause 1379. The on-body device of clause 1378, wherein the on-body device comprises filling material in the recess.

Clause 1370. The on-body device of any of clauses 1 to 154, 178 to 288, 322 to 323, 324 to 610, or 1370 to 1373, wherein the on-body device comprises a housing with a recess configured to receive sensor electronics, wherein the recess is arranged in a top of the housing.

Clause 1371. An on-body device, comprising: a sensor; electronics configured to receive a signal from the sensor, the signal indicative of a level of an analyte of a user; a housing configured to retain the electronics; and an adhesive patch adhesively coupled with the housing, wherein the sensor comprises: a proximal portion configured to be positioned above skin of the user and to be electrically coupled with the electronics; and a distal portion configured to be transcutaneously positioned through the user's skin and in contact with a bodily fluid of the user, wherein the distal portion of the sensor is further configured to detect an analyte in the bodily fluid and output the signal to the electronics; wherein the housing is adapted to permit egress of moisture from an area adjacent the housing and the housing comprises a base having a first surface for facing a skin surface of the user, wherein the housing comprises one or more first channels along the first surface and one or more second channels along the first surface, wherein the one or more first channels and the one or more second channels are configured to permit egress of moisture from an area adjacent the first surface, and wherein the one or more second channels comprises a curved channel having two ends that terminate at a peripheral edge of the base.

Clause 1372. The on-body device of clause 1371, wherein the base comprises a first region, wherein the first region comprises an aperture in the housing.

Clause 1373. The on-body device of clause 1372, wherein the first region of the base is spaced apart from the peripheral edge of the base.

Clause 1374. The on-body device of any of clauses 1371 to 1373, wherein a depth of each of the one or more first channels and/or each of the one or more second channels varies along at least a portion of a length of the respective channel.

Clause 1375. The on-body device of any of clauses 1372 to 1373, wherein the depth of each of the one or more first channels decreases as each respective first channel progresses toward the first region of the base.

Clause 1376. The on-body device of clause 1375, wherein the depth of each of the one or more first channels decreases to zero as the respective first channel progresses toward the first region of the base.

Clause 1377. The on-body device of clause 1376, wherein each of the one or more first channels includes a tapered edge and/or a beveled edge where the respective channel meets the first region.

Clause 1378. The on-body device of clause 1374, wherein the depth of each of the one or more first channels and/or each of the one or more second channels varies in a stepped fashion.

Clause 1379. The on-body device of any of clauses 1371 to 1375, wherein the depth of each of the one or more first channels and/or each of the one or more second channels decreases with a radiused sloping edge.

Clause 1380. The on-body device of clause 1374, wherein the depth of each of the one or more first channels and/or each of the one or more second channels increases as the channel progresses towards the peripheral edge of the base.

Clause 1381. The on-body device of clause 1374, wherein a width of each of the one or more first channels and/or each of the one or more second channels narrows as the respective channel progresses towards the peripheral edge of the base.

Clause 1382. The on-body device of clause 1373, wherein a width of each of the one or more first channels and/or each of the one or more second channels narrows as the respective channel progresses towards the peripheral edge of the base.

Clause 1383. The on-body device of clause 1382, wherein the width of each of the one or more first channels and/or each of the one or more second channels tapers along a portion of the length of the respective channel at or adjacent the peripheral edge of the base.

Clause 1384. The on-body device of clause 1383, wherein each of the one or more first channels and/or each of the one or more second channels has a first width at a first location and a second width at a second location, and wherein the first width transitions to the second width in a stepped or continuous fashion.

Clause 1385. The on-body device of clause 1384, wherein a first width of each of the one or more first channels and/or each of the one or more second channels at the first location is less than a second width of the respective channel at the second location, and wherein a first depth of each of the one or more first channels and/or each of the one or more second channels at the first location is less than a second depth of the channel at the second location.

Clause 1386. The on-body device of any of clauses 1371 to 1385, wherein the first surface comprises a first proximal portion and a second proximal portion each having a contact surface for contact with the adhesive patch, wherein the first proximal portion and the second portion are defined, at least in part, by channels of the one or more first channels and/or the one or more second channels, and wherein the contact surface of the first proximal portion is 1.25 times as large or more in surface area than the contact surface of the second proximal portion, and wherein the first proximal portion does not extend to the peripheral edge of the base.

Clause 1387. The on-body device of any of clauses 1371 to 1386, wherein the first proximal portion does not extend to the peripheral edge of the base.

Clause 1388. The on-body device of any of clauses 1371 to 1387, wherein each of the one or more first channels and/or each of the one or more second channels have a cross-sectional profile that is U-shaped or V-shaped.

Clause 1389. The on-body device of any of clauses 1371 to 1388, wherein each of the one or more first channels and/or each of the one or more second channels has a maximum depth that is greater than 50 microns (μm) and less than 400 μm.

Clause 1390. The on-body device of any of clauses 1371 to 1388, wherein each of the one or more first channels and/or each of the one or more second channels has a width between 400 microns (μm) and 1000 μm.

As used herein, the terms “comprise”, “comprises”, and “comprising” are inclusive or open-ended and do not exclude additional, unrecited elements or method steps. The terms “include”, “includes”, and “including” are synonymous with “comprise”, “comprises”, and “comprising” unless stated otherwise. The terms “have”, “has”, and “having” are synonymous with “comprise”, “comprises”, and “comprising” unless stated otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1-112. (canceled)

113. An on-body device, comprising:
a sensor;
electronics configured to receive a signal from the sensor, the signal indicative of a level of an analyte of a user;
a housing configured to retain the electronics; and
an adhesive patch adhesively coupled with the housing,
wherein the sensor comprises:
a proximal portion configured to be positioned above skin of the user and to be electrically coupled with the electronics; and
a distal portion configured to be transcutaneously positioned through the user's skin and in contact with a bodily fluid of the user,
wherein the distal portion of the sensor is further configured to detect an analyte in the bodily fluid and output the signal to the electronics;
wherein the housing is adapted to permit egress of moisture from an area adjacent the housing and the housing comprises a base having a first surface for facing a skin surface of the user, wherein the housing comprises one or more first channels along the first surface and one or more second channels along the first surface, wherein the one or more first channels and the one or more second channels are configured to permit egress of moisture from an area adjacent the first surface, and
wherein the one or more second channels comprises a curved channel having two ends that terminate at a peripheral edge of the base.

114. The on-body device of claim 113, wherein the base comprises a first region, wherein the first region comprises an aperture in the housing.

115. The on-body device of claim 114, wherein the first region of the base is spaced apart from the peripheral edge of the base.

116. The on-body device of claim 115, wherein a depth of each of the one or more first channels and/or each of the one or more second channels varies along at least a portion of a length of the respective channel.

117. The on-body device of claim 116, wherein the depth of each of the one or more first channels decreases as each respective first channel progresses toward the first region of the base.

118. The on-body device of claim 117, wherein the depth of each of the one or more first channels decreases to zero as the respective first channel progresses toward the first region of the base.

119. The on-body device of claim 118, wherein each of the one or more first channels includes a tapered edge and/or a beveled edge where the respective channel meets the first region.

120. The on-body device of claim 116, wherein the depth of each of the one or more first channels and/or each of the one or more second channels varies in a stepped fashion.

121. The on-body device of claim 116, wherein the depth of each of the one or more first channels and/or each of the one or more second channels decreases with a radiused sloping edge.

122. The on-body device of claim 116, wherein the depth of each of the one or more first channels and/or each of the one or more second channels increases as the channel progresses towards the peripheral edge of the base.

123. The on-body device of claim 116, wherein a width of each of the one or more first channels and/or each of the one or more second channels narrows as the respective channel progresses towards the peripheral edge of the base.

124. The on-body device of claim 115, wherein a width of each of the one or more first channels and/or each of the one or more second channels narrows as the respective channel progresses towards the peripheral edge of the base.

125. The on-body device of claim 124, wherein the width of each of the one or more first channels and/or each of the one or more second channels tapers along a portion of the length of the respective channel at or adjacent the peripheral edge of the base.

126. The on-body device of any of claim 125, wherein each of the one or more first channels and/or each of the one or more second channels has a first width at a first location and a second width at a second location, and wherein the first width transitions to the second width in a stepped or continuous fashion.

127. The on-body device of claim 126, wherein a first width of each of the one or more first channels and/or each of the one or more second channels at the first location is less than a second width of the respective channel at the second location, and wherein a first depth of each of the one or more first channels and/or each of the one or more second channels at the first location is less than a second depth of the channel at the second location.

128. The on-body device of claim 113, wherein the first surface comprises a first proximal portion and a second proximal portion each having a contact surface for contact with the adhesive patch, wherein the first proximal portion and the second portion are defined, at least in part, by channels of the one or more first channels and/or the one or more second channels, and wherein the contact surface of the first proximal portion is 1.25 times as large or more in surface area than the contact surface of the second proximal portion, and wherein the first proximal portion does not extend to the peripheral edge of the base.

129. The on-body device of claim 113, wherein the first proximal portion does not extend to the peripheral edge of the base.

130. The on-body device of claim 113, wherein each of the one or more first channels and/or each of the one or more second channels have a cross-sectional profile that is U-shaped or V-shaped.

131. The on-body device of claim 113, wherein each of the one or more first channels and/or each of the one or more second channels has a maximum depth that is greater than 50 microns (μm) and less than 400 μm.

132. The on-body device of claim 113, wherein each of the one or more first channels and/or each of the one or more second channels has a width between 400 microns (μm) and 1000 μm.
